(19) United States
(12) Plant Patent Application Publication
(10) Pub. No.: US 2015/0359150 P1
(43) Pub. Date: Dec. 10, 2015

(54) Strawberry cultivars

(50) ;

(71) Applicant: The Regents of the University of California, Oakland, CA (US)

(72) Inventors: Douglas V. Shaw, Davis, CA (US); Kirk D. Larson, Santa Ana, CA (US)

(73) Assignee: The Regents of the University of California, Oakland, CA (US)

(21) Appl. No.: 14/545,653

(22) Filed: Jun. 3, 2015

Related U.S. Application Data
(60) Provisional application No. 61/999,632, filed on Jul. 30, 2014.
(60) Provisional application No. 61/997,548, filed on Jun. 4, 2014.

(51) Int. Cl. A01H 5/00

(52) U.S. Cl. PLT/208

(57) ABSTRACT

The invention relates to new strawberry cultivars (Fragaria×ananassa Duch.) which have distinct disease/pathogen resistance, yield, appearance, size, and firmness, and which display desirable plant and fruit qualities.

### CROSS-REFERENCE TO RELATED APPLICATIONS

This application claims the benefit of U.S. Provisional Application No. 61/997,548, filed Jun. 4, 2014, and U.S. Provisional Application No. 61/999,632, filed Jul. 30, 2014, both of which are hereby incorporated by reference in their entirety.

### LATIN NAME

Genus and species: The strawberry cultivars of this invention are botanically identified as Fragaria×ananassa Duch.

### VARIETAL DENOMINATION

Variety denomination: The variety denominations are ‘01.80-602’, ‘01.138-1’, ‘03.132-3’, ‘04.9-5’, ‘04.18-4’, ‘04.41-6’, ‘04.41-7’, ‘04.76-4’, ‘04.77-2’, ‘05.92-11’, ‘05.95-4’, ‘05.99-6’, ‘05.107-2’, ‘05.108-7’, ‘05.109-2’, ‘05.117-5’, ‘05.120-9’, ‘05.132-1’, ‘05.148-2’, ‘05.165-1’, ‘05.197-2’, ‘05.205-2’, ‘05.206-5’, ‘06.16-602’, ‘06.20-605’, ‘06.51-609’, ‘06.137-2’, ‘06.204-1’, ‘07.1-608’, ‘07.9-601’, ‘07.64-2’, ‘07.64-3’, ‘07.64-5’, ‘07.67-608’, ‘07.90-606’, ‘07.92-3’, ‘07.120-3’, ‘07.134-1’, ‘07.148-1’, ‘07.148-3’, ‘08.5-601’, ‘08.13-605’, ‘08.17-5’, ‘08.18-5’, ‘08.37-603’, ‘08.43-1’, ‘08.43-4’, ‘08.75-1’, ‘08.114-5’, ‘08.123-1’, ‘08.124-1’, ‘08.128-2’, ‘08.129-4’, ‘08.131-3’, ‘08.132-3’, ‘08.137-1’, ‘08.138-3’, ‘08.150-9’, ‘09.12-605’, ‘09.16-601’, ‘09.20-609’, ‘09.32-605’, ‘09.37-1’, ‘09.48-3’, ‘09.56-608’, ‘09.61-602’, ‘09.62-1’, ‘09.68-602’, ‘09.78-603’, ‘09.85-2’, ‘09.85-601’, ‘09.88-602’, ‘09.91-1’, ‘09.97-3’, ‘09.100-3’, ‘09.103-2’, ‘09.108-2’, ‘09.132-3’, ‘09.141-1’, ‘09.154-605’, ‘09.166-7’, ‘09.171-602’, ‘09.180-1’, ‘10.1-604’, ‘10.5-602’, ‘10.7-3’, ‘10.12-3’, ‘10.15-605’, ‘10.17-1’, ‘10.17-2’, ‘10.23-1’, ‘10.24-2’, ‘10.25-601’, ‘10.30-1’, ‘10.31-602’, ‘10.37-604’, ‘10.38-601’, ‘10.70-611’, ‘10.77-601’, ‘10.78-603’, ‘10.80-605’, ‘10.81-1’, ‘10.82-601’, ‘10.87-2’, ‘10.99-1’, ‘10.105-1’, ‘10.106-1’, ‘10.106-8’, ‘10.116-1’, ‘10.122-3’, ‘10.122-6’, ‘10.124-2’, ‘10.126-3’, ‘10.127-1’, ‘10.134-4’, ‘10.136-4’, ‘10.137-5’, ‘10.140-1’, ‘10.141-1’, ‘10.143-1’, ‘10.144-1’, ‘10.144-2’, ‘10.144-3’, ‘10.160-1’, ‘10.160-9’, ‘10.169-1’, ‘10.187-4’, ‘11.21-1’, ‘11.32-2’, ‘11.34-605’, ‘11.36-601’, ‘11.39-1’, ‘11.49-6’, ‘11.50-1’, ‘11.51-1’, ‘11.55-4’, ‘11.57-1’, ‘11.62-603’, ‘11.67-1’, ‘11.71-601’, ‘11.80-2’, ‘11.103-1’, ‘11.103-3’, ‘11.103-5’, ‘11.105-1’, ‘11107-5’, ‘11.109-2’, ‘11.109-3’, ‘11.110-3’, ‘11.113-6’, ‘11.115-3’, ‘11.116-1’, ‘11.129-2’, ‘11.141-1’, ‘11.145-1’, ‘11.151-3’, ‘11.151-8’, ‘11.153-2’, ‘11.153-3’, ‘11.154-5’, ‘11.157-1’, ‘11.158-1’, ‘11.167-4’, ‘11.179-3’, ‘11.180-1’, ‘11.181-2’, ‘11.190-3’, and ‘11.195-3’.

### BACKGROUND OF THE INVENTION

Modern cultivated strawberry is a hybrid species of the genus Fragaria that is cultivated worldwide for its fruit. Modern cultivated strawberry was first bred in Brittany, France, in the 18^{th} century by crossing Fragaria virginiana (native to eastern North America) with Fragaria chiloensis (native to Chile) The strawberry is an aggregate accessory fruit, meaning that the fleshy part of the fruit is derived not from the plant's ovaries but from the receptacle that holds the ovaries. Each apparent “seed” on the outside of the fruit is actually one of the ovaries of the flower, with a seed inside it.

Strawberry cultivars vary widely in size, color, flavor, shape, degree of fertility, season of ripening, susceptibility to disease, and constitution of plant. Certain cultivars vary in foliage, and some vary materially in the relative development of their sexual organs. Generally, strawberry flowers appear hermaphroditic in structure, but function as either male or female. Typical commercial production of strawberry plants involves propagation from runners and distribution as either bare root plants or plugs. Cultivation is either annual plasticulture or perennial. Strawberries can also be produced in greenhouses during the off season.

Strawberry fruit is widely appreciated for its characteristic aroma, bright red color, juicy texture, and sweetness. Strawberry fruit is a popular fruit that is typically consumed either fresh or in prepared foods, such as preserves, fruit juice, and baked goods.

Strawberry is an important and valuable fruit crop. Accordingly, there is a need for new strawberry cultivars. In particular, there is a need for improved strawberry cultivars that are stable, high yielding, and agronomically sound.

### SUMMARY OF THE INVENTION

In order to meet these needs, the present invention is directed to improved strawberry cultivars. In particular, the invention relates to new and distinctive day-neutral type cultivars and short-day type cultivars. The new strawberry cultivars were chosen for their disease/pathogen resistance, yield, appearance, size, and firmness.

The new cultivars of the present invention were first fruited at the at the University of California Wolfskill Experimental Orchard near Winters, Calif., or at the University of California South Coast Research and Extension Center near Irvine, Calif., where each of the cultivars was selected and propagated by runners. Asexual propagules from the original source of each new cultivar have been tested at the University of California Strawberry Research Facility near Watsonville, Calif., and at the University of California South Coast Research and Extension Center near Irvine, Calif. The properties of each new cultivar of the present invention were found to be transmissible by such asexual reproduction. Each new strawberry cultivar of the present invention is stable and reproduces true to type in successive generations of asexual reproduction.

Each new strawberry cultivar of the present invention finds use, for example, as a parental line for breeding purposes.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the plant of strawberry cultivar ‘01.80-602’.

FIG. 2 shows fruit of strawberry cultivar ‘01.80-602’.

FIG. 3 shows the flower of strawberry cultivar ‘01.80-602’.

FIG. 4 shows the plant of strawberry cultivar ‘01.138-1’.

FIG. 5 shows fruit of strawberry cultivar ‘01.138-1’.

FIG. 6 shows the flower of strawberry cultivar ‘01.138-1’.

FIG. 7 shows the plant of strawberry cultivar ‘03.132-3’.

FIG. 8 shows fruit of strawberry cultivar ‘03.132-3’.

FIG. 9 shows the flower of strawberry cultivar ‘03.132-3’.

FIG. 10 shows the plant of strawberry cultivar ‘04.9-5’.

FIG. 11 shows fruit of strawberry cultivar ‘04.9-5’.

FIG. 12 shows the flower of strawberry cultivar ‘04.9-5’.

FIG. 13 shows the plant of strawberry cultivar ‘04.18-4’.

FIG. 14 shows fruit of strawberry cultivar ‘04.18-4’.

FIG. 15 shows the flower of strawberry cultivar ‘04.18-4’.

FIG. 16 shows the plant of strawberry cultivar ‘04.41-6’.

FIG. 17 shows fruit of strawberry cultivar ‘04.41-6’.

FIG. 18 shows the flower of strawberry cultivar ‘04.41-6’.

FIG. 19 shows the plant of strawberry cultivar ‘04.41-7’.

FIG. 20 shows fruit of strawberry cultivar ‘04.41-7’.

FIG. 21 shows the flower of strawberry cultivar ‘04.41-7’.

FIG. 22 shows the plant of strawberry cultivar ‘04.76-4’.

FIG. 23 shows fruit of strawberry cultivar ‘04.76-4’.

FIG. 24 shows the flower of strawberry cultivar ‘04.76-4’.

FIG. 25 shows the plant of strawberry cultivar ‘04.77-2’.

FIG. 26 shows fruit of strawberry cultivar ‘04.77-2’.

FIG. 27 shows the flower of strawberry cultivar ‘04.77-2’.

FIG. 28 shows the plant of strawberry cultivar ‘05.92-11’.

FIG. 29 shows fruit of strawberry cultivar ‘05.92-11’.

FIG. 30 shows the flower of strawberry cultivar ‘05.92-11’.

FIG. 31 shows the plant of strawberry cultivar ‘05.95-4’.

FIG. 32 shows fruit of strawberry cultivar ‘05.95-4’.

FIG. 33 shows the flower of strawberry cultivar ‘05.95-4’.

FIG. 34 shows the plant of strawberry cultivar ‘05.99-6’.

FIG. 35 shows fruit of strawberry cultivar ‘05.99-6’.

FIG. 36 shows the flower of strawberry cultivar ‘05.99-6’.

FIG. 37 shows the plant of strawberry cultivar ‘05.107-2’.

FIG. 38 shows fruit of strawberry cultivar ‘05.107-2’.

FIG. 39 shows the flower of strawberry cultivar ‘05.107-2’.

FIG. 40 shows the plant of strawberry cultivar ‘05.108-7’.

FIG. 41 shows fruit of strawberry cultivar ‘05.108-7’.

FIG. 42 shows the flower of strawberry cultivar ‘05.108-7’.

FIG. 43 shows the plant of strawberry cultivar ‘05.109-2’.

FIG. 44 shows fruit of strawberry cultivar ‘05.109-2’.

FIG. 45 shows the flower of strawberry cultivar ‘05.109-2’.

FIG. 46 shows the plant of strawberry cultivar ‘05.117-5’.

FIG. 47 shows fruit of strawberry cultivar ‘05.117-5’.

FIG. 48 shows the flower of strawberry cultivar ‘05.117-5’.

FIG. 49 shows the plant of strawberry cultivar ‘05.120-9’.

FIG. 50 shows fruit of strawberry cultivar ‘05.120-9’.

FIG. 51 shows the flower of strawberry cultivar ‘05.120-9’.

FIG. 52 shows the plant of strawberry cultivar ‘05.132-1’.

FIG. 53 shows fruit of strawberry cultivar ‘05.132-1’.

FIG. 54 shows the flower of strawberry cultivar ‘05.132-1’.

FIG. 55 shows the plant of strawberry cultivar ‘05.148-2’.

FIG. 56 shows fruit of strawberry cultivar ‘05.148-2’.

FIG. 57 shows the flower of strawberry cultivar ‘05.148-2’.

FIG. 58 shows the plant of strawberry cultivar ‘05.165-1’.

FIG. 59 shows fruit of strawberry cultivar ‘05.165-1’.

FIG. 60 shows the flower of strawberry cultivar ‘05.165-1’.

FIG. 61 shows the plant of strawberry cultivar ‘05.197-2’.

FIG. 62 shows fruit of strawberry cultivar ‘05.197-2’.

FIG. 63 shows the flower of strawberry cultivar ‘05.197-2’.

FIG. 64 shows the plant of strawberry cultivar ‘05.205-2’.

FIG. 65 shows fruit of strawberry cultivar ‘05.205-2’.

FIG. 66 shows the flower of strawberry cultivar ‘05.205-2’.

FIG. 67 shows the plant of strawberry cultivar ‘05.206-5’.

FIG. 68 shows fruit of strawberry cultivar ‘05.206-5’.

FIG. 69 shows the flower of strawberry cultivar ‘05.206-5’.

FIG. 70 shows the plant of strawberry cultivar ‘06.16-602’.

FIG. 71 shows fruit of strawberry cultivar ‘06.16-602’.

FIG. 72 shows the flower of strawberry cultivar ‘06.16-602’.

FIG. 73 shows the plant of strawberry cultivar ‘06.20-605’.

FIG. 74 shows fruit of strawberry cultivar ‘06.20-605’.

FIG. 75 shows the flower of strawberry cultivar ‘06.20-605’.

FIG. 76 shows the plant of strawberry cultivar ‘06.51-609’.

FIG. 77 shows fruit of strawberry cultivar ‘06.51-609’.

FIG. 78 shows the flower of strawberry cultivar ‘06.51-609’.

FIG. 79 shows the plant of strawberry cultivar ‘06.137-2’.

FIG. 80 shows fruit of strawberry cultivar ‘06.137-2’.

FIG. 81 shows the flower of strawberry cultivar ‘06.137-2’.

FIG. 82 shows the plant of strawberry cultivar ‘06.204-1’.

FIG. 83 shows fruit of strawberry cultivar ‘06.204-1’.

FIG. 84 shows the flower of strawberry cultivar ‘06.204-1’.

FIG. 85 shows the plant of strawberry cultivar ‘07.1-608’.

FIG. 86 shows fruit of strawberry cultivar ‘07.1-608’.

FIG. 87 shows the flower of strawberry cultivar ‘07.1-608’.

FIG. 88 shows the plant of strawberry cultivar ‘07.9-601’.

FIG. 89 shows fruit of strawberry cultivar ‘07.9-601’.

FIG. 90 shows the flower of strawberry cultivar ‘07.9-601’.

FIG. 91 shows the plant of strawberry cultivar ‘07.64-2’.

FIG. 92 shows fruit of strawberry cultivar ‘07.64-2’.

FIG. 93 shows the flower of strawberry cultivar ‘07.64-2’.

FIG. 94 shows the plant of strawberry cultivar ‘07.64-3’.

FIG. 95 shows fruit of strawberry cultivar ‘07.64-3’.

FIG. 96 shows the flower of strawberry cultivar ‘07.64-3’.

FIG. 97 shows the plant of strawberry cultivar ‘07.64-5’.

FIG. 98 shows fruit of strawberry cultivar ‘07.64-5’.

FIG. 99 shows the flower of strawberry cultivar ‘07.64-5’.

FIG. 100 shows the plant of strawberry cultivar ‘07.67-608’.

FIG. 101 shows fruit of strawberry cultivar ‘07.67-608’.

FIG. 102 shows the flower of strawberry cultivar ‘07.67-608’.

FIG. 103 shows the plant of strawberry cultivar ‘07.90-606’.

FIG. 104 shows fruit of strawberry cultivar ‘07.90-606’.

FIG. 105 shows the flower of strawberry cultivar ‘07.90-606’.

FIG. 106 shows the plant of strawberry cultivar ‘07.92-3’.

FIG. 107 shows fruit of strawberry cultivar ‘07.92-3’.

FIG. 108 shows the flower of strawberry cultivar ‘07.92-3’.

FIG. 109 shows the plant of strawberry cultivar ‘07.120-3’.

FIG. 110 shows fruit of strawberry cultivar ‘07.120-3’.

FIG. 111 shows the flower of strawberry cultivar 07.120-3’.

FIG. 112 shows the plant of strawberry cultivar ‘07.134-1’.

FIG. 113 shows fruit of strawberry cultivar ‘07.134-1’.

FIG. 114 shows the flower of strawberry cultivar ‘07.134-1’.

FIG. 115 shows the plant of strawberry cultivar ‘07.148-1’.

FIG. 116 shows fruit of strawberry cultivar ‘07.148-1’.

FIG. 117 shows the flower of strawberry cultivar ‘07.148-1’.

FIG. 118 shows the plant of strawberry cultivar ‘07.148-3’.

FIG. 119 shows fruit of strawberry cultivar ‘07.148-3’.

FIG. 120 shows the flower of strawberry cultivar ‘07.148-3’.

FIG. 121 shows the plant of strawberry cultivar ‘08.5-601’.

FIG. 122 shows fruit of strawberry cultivar ‘08.5-601’.

FIG. 123 shows the flower of strawberry cultivar ‘08.5-601’.

FIG. 124 shows the plant of strawberry cultivar ‘08.13-605’.

FIG. 125 shows fruit of strawberry cultivar ‘08.13-605’.

FIG. 126 shows the flower of strawberry cultivar ‘08.13-605’.

FIG. 127 shows the plant of strawberry cultivar ‘08.17-5’.

FIG. 128 shows fruit of strawberry cultivar ‘08.17-5’.

FIG. 129 shows the flower of strawberry cultivar ‘08.17-5’.

FIG. 130 shows the plant of strawberry cultivar ‘08.18-5’.

FIG. 131 shows fruit of strawberry cultivar ‘08.18-5’.

FIG. 132 shows the flower of strawberry cultivar ‘08.18-5’.

FIG. 133 shows the plant of strawberry cultivar ‘08.37-603’.

FIG. 134 shows fruit of strawberry cultivar ‘08.37-603’.

FIG. 135 shows the flower of strawberry cultivar ‘08.37-603’.

FIG. 136 shows the plant of strawberry cultivar ‘08.43-1’.

FIG. 137 shows fruit of strawberry cultivar ‘08.43-1’.

FIG. 138 shows the flower of strawberry cultivar ‘08.43-1’.

FIG. 139 shows the plant of strawberry cultivar ‘08.43-4’.

FIG. 140 shows fruit of strawberry cultivar ‘08.43-4’.

FIG. 141 shows the flower of strawberry cultivar ‘08.43-4’.

FIG. 142 shows the plant of strawberry cultivar ‘08.75-1’.

FIG. 143 shows fruit of strawberry cultivar ‘08.75-1’.

FIG. 144 shows the flower of strawberry cultivar ‘08.75-1’.

FIG. 145 shows the plant of strawberry cultivar ‘08.114-5’.

FIG. 146 shows fruit of strawberry cultivar ‘08.114-5’.

FIG. 147 shows the flower of strawberry cultivar ‘08.114-5’.

FIG. 148 shows the plant of strawberry cultivar ‘08.123-1’.

FIG. 149 shows fruit of strawberry cultivar ‘08.123-1’.

FIG. 150 shows the flower of strawberry cultivar ‘08.123-1’.

FIG. 151 shows the plant of strawberry cultivar ‘08.124-1’.

FIG. 152 shows fruit of strawberry cultivar ‘08.124-1’.

FIG. 153 shows the flower of strawberry cultivar ‘08.124-1’.

FIG. 154 shows the plant of strawberry cultivar ‘08.128-2’.

FIG. 155 shows fruit of strawberry cultivar ‘08.128-2’.

FIG. 156 shows the flower of strawberry cultivar ‘08.128-2’.

FIG. 157 shows the plant of strawberry cultivar ‘08.129-4’.

FIG. 158 shows fruit of strawberry cultivar ‘08.129-4’.

FIG. 159 shows the flower of strawberry cultivar ‘08.129-4’.

FIG. 160 shows the plant of strawberry cultivar ‘08.131-3’.

FIG. 161 shows fruit of strawberry cultivar ‘08.131-3’.

FIG. 162 shows the flower of strawberry cultivar ‘08.131-3’.

FIG. 163 shows the plant of strawberry cultivar ‘08.132-3’.

FIG. 164 shows fruit of strawberry cultivar ‘08.132-3’.

FIG. 165 shows the flower of strawberry cultivar ‘08.132-3’.

FIG. 166 shows the plant of strawberry cultivar ‘08.137-1’.

FIG. 167 shows fruit of strawberry cultivar ‘08.137-1’.

FIG. 168 shows the flower of strawberry cultivar ‘08.137-1’.

FIG. 169 shows the plant of strawberry cultivar ‘08.138-3’.

FIG. 170 shows fruit of strawberry cultivar ‘08.138-3’.

FIG. 171 shows the flower of strawberry cultivar ‘08.138-3’.

FIG. 172 shows the plant of strawberry cultivar ‘08.150-9’.

FIG. 173 shows fruit of strawberry cultivar ‘08.150-9’.

FIG. 174 shows the flower of strawberry cultivar ‘08.150-9’.

FIG. 175 shows the plant of strawberry cultivar ‘09.12-605’.

FIG. 176 shows fruit of strawberry cultivar ‘09.12-605’.

FIG. 177 shows the flower of strawberry cultivar ‘09.12-605’.

FIG. 178 shows the plant of strawberry cultivar ‘09.16-601’.

FIG. 179 shows fruit of strawberry cultivar ‘09.16-601’.

FIG. 180 shows the flower of strawberry cultivar ‘09.16-601’.

FIG. 181 shows the plant of strawberry cultivar ‘09.20-609’.

FIG. 182 shows fruit of strawberry cultivar ‘09.20-609’.

FIG. 183 shows the flower of strawberry cultivar ‘09.20-609’.

FIG. 184 shows the plant of strawberry cultivar ‘09.32-605’.

FIG. 185 shows fruit of strawberry cultivar ‘09.32-605’.

FIG. 186 shows the flower of strawberry cultivar ‘09.32-605’.

FIG. 187 shows the plant of strawberry cultivar ‘09.37-1’.

FIG. 188 shows fruit of strawberry cultivar ‘09.37-1’.

FIG. 189 shows the flower of strawberry cultivar ‘09.37-1’.

FIG. 190 shows the plant of strawberry cultivar ‘09.48-3’.

FIG. 191 shows fruit of strawberry cultivar ‘09.48-3’.

FIG. 192 shows the flower of strawberry cultivar ‘09.48-3’.

FIG. 193 shows the plant of strawberry cultivar ‘09.56-608’.

FIG. 194 shows fruit of strawberry cultivar ‘09.56-608’.

FIG. 195 shows the flower of strawberry cultivar ‘09.56-608’.

FIG. 196 shows the plant of strawberry cultivar ‘09.61-602’.

FIG. 197 shows fruit of strawberry cultivar ‘09.61-602’.

FIG. 198 shows the flower of strawberry cultivar ‘09.61-602’.

FIG. 199 shows the plant of strawberry cultivar ‘09.62-1’.

FIG. 200 shows fruit of strawberry cultivar ‘09.62-1’.

FIG. 201 shows the flower of strawberry cultivar ‘09.62-1’.

FIG. 202 shows the plant of strawberry cultivar ‘09.68-602’.

FIG. 203 shows fruit of strawberry cultivar ‘09.68-602’.

FIG. 204 shows the flower of strawberry cultivar ‘09.68-602’.

FIG. 205 shows the plant of strawberry cultivar ‘09.78-603’.

FIG. 206 shows fruit of strawberry cultivar ‘09.78-603’.

FIG. 207 shows the flower of strawberry cultivar ‘09.78-603’.

FIG. 208 shows the plant of strawberry cultivar ‘09.85-2’.

FIG. 209 shows fruit of strawberry cultivar ‘09.85-2’.

FIG. 210 shows the flower of strawberry cultivar ‘09.85-2’.

FIG. 211 shows the plant of strawberry cultivar ‘09.85-601’.

FIG. 212 shows fruit of strawberry cultivar ‘09.85-601’.

FIG. 213 shows the flower of strawberry cultivar ‘09.85-601’.

FIG. 214 shows the plant of strawberry cultivar ‘09.88-602’.

FIG. 215 shows fruit of strawberry cultivar ‘09.88-602’.

FIG. 216 shows the flower of strawberry cultivar ‘09.88-602’.

FIG. 217 shows the plant of strawberry cultivar ‘09.91-1’.

FIG. 218 shows fruit of strawberry cultivar ‘09.91-1’.

FIG. 219 shows the flower of strawberry cultivar ‘09.91-1’.

FIG. 220 shows the plant of strawberry cultivar ‘09.97-3’.

FIG. 221 shows fruit of strawberry cultivar ‘09.97-3’.

FIG. 222 shows the flower of strawberry cultivar ‘09.97-3’.

FIG. 223 shows the plant of strawberry cultivar ‘09.100-3’.

FIG. 224 shows fruit of strawberry cultivar ‘09.100-3’.

FIG. 225 shows the flower of strawberry cultivar ‘09.100-3’.

FIG. 226 shows the plant of strawberry cultivar ‘09.103-2’.

FIG. 227 shows fruit of strawberry cultivar ‘09.103-2’.

FIG. 228 shows the flower of strawberry cultivar ‘09.103-2’.

FIG. 229 shows the plant of strawberry cultivar ‘09.108-2’.

FIG. 230 shows fruit of strawberry cultivar ‘09.108-2’.

FIG. 231 shows the flower of strawberry cultivar ‘09.108-2’.

FIG. 232 shows the plant of strawberry cultivar ‘09.132-3’.

FIG. 233 shows fruit of strawberry cultivar ‘09.132-3’.

FIG. 234 shows the flower of strawberry cultivar ‘09.132-3’.

FIG. 235 shows the plant of strawberry cultivar ‘09.141-1’.

FIG. 236 shows fruit of strawberry cultivar ‘09.141-1’.

FIG. 237 shows the flower of strawberry cultivar ‘09.141-1’.

FIG. 238 shows the plant of strawberry cultivar ‘09.154-605’.

FIG. 239 shows fruit of strawberry cultivar ‘09.154-605’.

FIG. 240 shows the flower of strawberry cultivar ‘09.154-605’.

FIG. 241 shows the plant of strawberry cultivar ‘09.166-7’.

FIG. 242 shows fruit of strawberry cultivar ‘09.166-7’.

FIG. 243 shows the flower of strawberry cultivar ‘09.166-7’.

FIG. 244 shows the plant of strawberry cultivar ‘09.171-602’.

FIG. 245 shows fruit of strawberry cultivar ‘09.171-602’.

FIG. 246 shows the flower of strawberry cultivar ‘09.171-602’.

FIG. 247 shows the plant of strawberry cultivar ‘09.180-1’.

FIG. 248 shows fruit of strawberry cultivar ‘09.180-1’.

FIG. 249 shows the flower of strawberry cultivar ‘09.180-1’.

FIG. 250 shows the plant of strawberry cultivar ‘10.1-604’.

FIG. 251 shows fruit of strawberry cultivar ‘10.1-604’.

FIG. 252 shows the flower of strawberry cultivar ‘10.1-604’.

FIG. 253 shows the plant of strawberry cultivar ‘10.5-602’.

FIG. 254 shows fruit of strawberry cultivar ‘10.5-602’.

FIG. 255 shows the flower of strawberry cultivar ‘10.5-602’.

FIG. 256 shows the plant of strawberry cultivar ‘10.7-3’.

FIG. 257 shows fruit of strawberry cultivar ‘10.7-3’.

FIG. 258 shows the flower of strawberry cultivar ‘10.7-3’.

FIG. 259 shows the plant of strawberry cultivar ‘10.12-3’.

FIG. 260 shows fruit of strawberry cultivar ‘10.12-3’.

FIG. 261 shows the flower of strawberry cultivar ‘10.12-3’.

FIG. 262 shows the plant of strawberry cultivar ‘10.15-605’.

FIG. 263 shows fruit of strawberry cultivar ‘10.15-605’.

FIG. 264 shows the flower of strawberry cultivar ‘10.15-605’.

FIG. 265 shows the plant of strawberry cultivar ‘10.17-1’.

FIG. 266 shows fruit of strawberry cultivar ‘10.17-1’.

FIG. 267 shows the flower of strawberry cultivar ‘10.17-1’.

FIG. 268 shows the plant of strawberry cultivar ‘10.17-2’.

FIG. 269 shows fruit of strawberry cultivar ‘10.17-2’.

FIG. 270 shows the flower of strawberry cultivar ‘10.17-2’.

FIG. 271 shows the plant of strawberry cultivar ‘10.23-1’.

FIG. 272 shows fruit of strawberry cultivar ‘10.23-1’.

FIG. 273 shows the flower of strawberry cultivar ‘10.23-1’.

FIG. 274 shows the plant of strawberry cultivar ‘10.24-2’.

FIG. 275 shows fruit of strawberry cultivar ‘10.24-2’.

FIG. 276 shows the flower of strawberry cultivar ‘10.24-2’.

FIG. 277 shows the plant of strawberry cultivar ‘10.25-601’.

FIG. 278 shows fruit of strawberry cultivar ‘10.25-601’.

FIG. 279 shows the flower of strawberry cultivar ‘10.25-601’.

FIG. 280 shows the plant of strawberry cultivar ‘10.30-1’.

FIG. 281 shows fruit of strawberry cultivar ‘10.30-1’.

FIG. 282 shows the flower of strawberry cultivar ‘10.30-1’.

FIG. 283 shows the plant of strawberry cultivar ‘10.31-602’.

FIG. 284 shows fruit of strawberry cultivar ‘10.31-602’.

FIG. 285 shows the flower of strawberry cultivar ‘10.31-602’.

FIG. 286 shows the plant of strawberry cultivar ‘10.37-604’.

FIG. 287 shows fruit of strawberry cultivar ‘10.37-604’.

FIG. 288 shows the flower of strawberry cultivar ‘10.37-604’.

FIG. 289 shows the plant of strawberry cultivar ‘10.38-601’.

FIG. 290 shows fruit of strawberry cultivar ‘10.38-601’.

FIG. 291 shows the flower of strawberry cultivar ‘10.38-601’.

FIG. 292 shows the plant of strawberry cultivar ‘10.70-611’.

FIG. 293 shows fruit of strawberry cultivar ‘10.70-611’.

FIG. 294 shows the flower of strawberry cultivar ‘10.70-611’.

FIG. 295 shows the plant of strawberry cultivar ‘10.77-601’.

FIG. 296 shows fruit of strawberry cultivar ‘10.77-601’.

FIG. 297 shows the flower of strawberry cultivar ‘10.77-601’.

FIG. 298 shows the plant of strawberry cultivar ‘10.78-603’.

FIG. 299 shows fruit of strawberry cultivar ‘10.77-601’.

FIG. 300 shows the flower of strawberry cultivar ‘10.77-601’.

FIG. 301 shows the plant of strawberry cultivar ‘10.80-605’.

FIG. 302 shows fruit of strawberry cultivar ‘10.80-605’.

FIG. 303 shows the flower of strawberry cultivar ‘10.80-605’.

FIG. 304 shows the plant of strawberry cultivar ‘10.81-1’.

FIG. 305 shows fruit of strawberry cultivar ‘10.81-1’.

FIG. 306 shows the flower of strawberry cultivar ‘10.81-1’.

FIG. 307 shows the plant of strawberry cultivar ‘10.82-601’.

FIG. 308 shows fruit of strawberry cultivar ‘10.82-601’.

FIG. 309 shows the flower of strawberry cultivar ‘10.82-601’.

FIG. 310 shows the plant of strawberry cultivar ‘10.87-2’.

FIG. 311 shows fruit of strawberry cultivar ‘10.87-2’.

FIG. 312 shows the flower of strawberry cultivar ‘10.87-2’.

FIG. 313 shows the plant of strawberry cultivar ‘10.99-1’.

FIG. 314 shows fruit of strawberry cultivar ‘10.87-2’.

FIG. 315 shows the flower of strawberry cultivar ‘10.87-2’.

FIG. 316 shows the plant of strawberry cultivar ‘10.105-1’.

FIG. 317 shows fruit of strawberry cultivar ‘10.105-1’.

FIG. 318 shows the flower of strawberry cultivar ‘10.105-1’.

FIG. 319 shows the plant of strawberry cultivar ‘10.106-1’.

FIG. 320 shows fruit of strawberry cultivar ‘10.106-1’.

FIG. 321 shows the flower of strawberry cultivar ‘10.106-1’.

FIG. 322 shows the plant of strawberry cultivar ‘10.106-8’.

FIG. 323 shows fruit of strawberry cultivar ‘10.106-8’.

FIG. 324 shows the flower of strawberry cultivar ‘10.106-8’.

FIG. 325 shows the plant of strawberry cultivar ‘10.116-1’.

FIG. 326 shows fruit of strawberry cultivar ‘10.116-1’.

FIG. 327 shows the flower of strawberry cultivar ‘10.116-1’.

FIG. 328 shows the plant of strawberry cultivar ‘10.122-3’.

FIG. 329 shows fruit of strawberry cultivar ‘10.122-3’.

FIG. 330 shows the flower of strawberry cultivar ‘10.122-3’.

FIG. 331 shows the plant of strawberry cultivar ‘10.122-6’.

FIG. 332 shows fruit of strawberry cultivar ‘10.122-6’.

FIG. 333 shows the flower of strawberry cultivar ‘10.122-6’.

FIG. 334 shows the plant of strawberry cultivar ‘10.124-2’.

FIG. 335 shows fruit of strawberry cultivar ‘10.124-2’.

FIG. 336 shows the flower of strawberry cultivar ‘10.124-2’.

FIG. 337 shows the plant of strawberry cultivar ‘10.126-3’.

FIG. 338 shows a leaf and stem of strawberry cultivar ‘10.126-3’.

FIG. 339 shows the flower of strawberry cultivar ‘10.126-3’.

FIG. 340 shows the plant of strawberry cultivar ‘10.127-1’.

FIG. 341 shows fruit of strawberry cultivar ‘10.127-1’.

FIG. 342 shows the flower of strawberry cultivar ‘10.127-1’.

FIG. 343 shows the plant of strawberry cultivar ‘10.134-4’.

FIG. 344 shows fruit of strawberry cultivar ‘10.134-4’.

FIG. 345 shows the flower of strawberry cultivar ‘10.134-4’.

FIG. 346 shows the plant of strawberry cultivar ‘10.136-4’.

FIG. 347 shows fruit of strawberry cultivar ‘10.136-4’.

FIG. 348 shows a leaf and stem of strawberry cultivar ‘10.136-4’.

FIG. 349 shows the plant of strawberry cultivar ‘10.137-5’.

FIG. 350 shows fruit of strawberry cultivar ‘10.137-5’.

FIG. 351 shows the flower of strawberry cultivar ‘10.137-5’.

FIG. 352 shows the plant of strawberry cultivar ‘10.140-1’.

FIG. 353 shows fruit of strawberry cultivar ‘10.140-1’.

FIG. 354 shows the flower of strawberry cultivar ‘10.140-1’.

FIG. 355 shows the plant of strawberry cultivar ‘10.141-1’.

FIG. 356 shows fruit of strawberry cultivar ‘10.141-1’.

FIG. 357 shows the flower of strawberry cultivar ‘10.141-1’.

FIG. 358 shows the plant of strawberry cultivar ‘10.143-1’.

FIG. 359 shows fruit of strawberry cultivar ‘10.143-1’.

FIG. 360 shows the flower of strawberry cultivar ‘10.143-1’.

FIG. 361 shows the plant of strawberry cultivar ‘10.144-1’.

FIG. 362 shows fruit of strawberry cultivar ‘10.144-1’.

FIG. 363 shows the flower of strawberry cultivar ‘10.144-1’.

FIG. 364 shows the plant of strawberry cultivar ‘10.144-2’.

FIG. 365 shows fruit of strawberry cultivar ‘10.144-2’.

FIG. 366 shows the flower of strawberry cultivar ‘10.144-2’.

FIG. 367 shows the plant of strawberry cultivar ‘10.144-3’.

FIG. 368 shows fruit of strawberry cultivar ‘10.144-3’.

FIG. 369 shows the flower of strawberry cultivar ‘10.144-3’.

FIG. 370 shows the plant of strawberry cultivar ‘10.160-1’.

FIG. 371 shows fruit of strawberry cultivar ‘10.160-1’.

FIG. 372 shows the flower of strawberry cultivar ‘10.160-1’.

FIG. 373 shows the plant of strawberry cultivar ‘10.160-9’.

FIG. 374 shows fruit of strawberry cultivar ‘10.160-9’.

FIG. 375 shows the flower of strawberry cultivar ‘10.160-9’.

FIG. 376 shows the plant of strawberry cultivar ‘10.169-1’.

FIG. 377 shows fruit of strawberry cultivar ‘10.169-1’.

FIG. 378 shows the flower of strawberry cultivar ‘10.169-1’.

FIG. 379 shows the plant of strawberry cultivar ‘10.187-4’.

FIG. 380 shows fruit of strawberry cultivar ‘10.187-4’.

FIG. 381 shows the flower of strawberry cultivar ‘10.187-4’.

FIG. 382 shows the plant of strawberry cultivar ‘11.21-1’.

FIG. 383 shows fruit of strawberry cultivar ‘11.21-1’.

FIG. 384 shows the flower of strawberry cultivar ‘11.21-1’.

FIG. 385 shows the plant of strawberry cultivar ‘11.32-2’.

FIG. 386 shows fruit of strawberry cultivar ‘11.32-2’.

FIG. 387 shows the flower of strawberry cultivar ‘11.32-2’.

FIG. 388 shows the plant of strawberry cultivar ‘11.34-605’.

FIG. 389 shows fruit of strawberry cultivar ‘11.34-605’.

FIG. 390 shows the flower of strawberry cultivar ‘11.34-605’.

FIG. 391 shows the plant of strawberry cultivar ‘11.36-601’.

FIG. 392 shows fruit of strawberry cultivar ‘11.36-601’.

FIG. 393 shows the flower of strawberry cultivar ‘11.36-601’.

FIG. 394 shows the plant of strawberry cultivar ‘11.39-1’.

FIG. 395 shows fruit of strawberry cultivar ‘11.39-1’.

FIG. 396 shows the flower of strawberry cultivar ‘11.39-1’.

FIG. 397 shows the plant of strawberry cultivar ‘11.49-6’.

FIG. 398 shows fruit of strawberry cultivar ‘11.49-6’.

FIG. 399 shows the flower of strawberry cultivar ‘11.49-6’.

FIG. 400 shows the plant of strawberry cultivar ‘11.50-1’.

FIG. 401 shows fruit of strawberry cultivar ‘11.50-1’.

FIG. 402 shows the flower of strawberry cultivar ‘11.50-1’.

FIG. 403 shows the plant of strawberry cultivar ‘11.51-1’.

FIG. 404 shows fruit of strawberry cultivar ‘11.51-1’.

FIG. 405 shows the flower of strawberry cultivar ‘11.51-1’.

FIG. 406 shows the plant of strawberry cultivar ‘11.55-4’.

FIG. 407 shows fruit of strawberry cultivar ‘11.55-4’.

FIG. 408 shows the flower of strawberry cultivar ‘11.55-4’.

FIG. 409 shows the plant of strawberry cultivar ‘11.57-1’.

FIG. 410 shows fruit of strawberry cultivar ‘11.57-1’.

FIG. 411 shows the flower of strawberry cultivar ‘11.57-1’.

FIG. 412 shows the plant of strawberry cultivar ‘11.62-603’.

FIG. 413 shows fruit of strawberry cultivar ‘11.62-603’.

FIG. 414 shows the flower of strawberry cultivar ‘11.62-603’.

FIG. 415 shows the plant of strawberry cultivar ‘11.67-1’.

FIG. 416 shows fruit of strawberry cultivar ‘11.67-1’.

FIG. 417 shows the flower of strawberry cultivar ‘11.67-1’.

FIG. 418 shows the plant of strawberry cultivar ‘11.71-601’.

FIG. 419 shows fruit of strawberry cultivar ‘11.71-601’.

FIG. 420 shows the flower of strawberry cultivar ‘11.71-601’.

FIG. 421 shows the plant of strawberry cultivar ‘11.80-2’.

FIG. 422 shows fruit of strawberry cultivar ‘11.80-2’.

FIG. 423 shows the flower of strawberry cultivar ‘11.80-2’.

FIG. 424 shows the plant of strawberry cultivar ‘11.103-1’.

FIG. 425 shows fruit of strawberry cultivar ‘11.103-1’.

FIG. 426 shows the flower of strawberry cultivar ‘11.103-1’.

FIG. 427 shows the plant of strawberry cultivar ‘11.103-3’.

FIG. 428 shows fruit of strawberry cultivar ‘11.103-3’.

FIG. 429 shows the flower of strawberry cultivar ‘11.103-3’.

FIG. 430 shows the plant of strawberry cultivar ‘11.103-5’.

FIG. 431 shows fruit of strawberry cultivar ‘11.103-5’.

FIG. 432 shows the flower of strawberry cultivar ‘11.103-5’.

FIG. 433 shows the plant of strawberry cultivar ‘11.105-1’.

FIG. 434 shows fruit of strawberry cultivar ‘11.105-1’.

FIG. 435 shows the flower of strawberry cultivar ‘11.105-1’.

FIG. 436 shows the plant of strawberry cultivar ‘11.107-5’.

FIG. 437 shows fruit of strawberry cultivar ‘11.107-5’.

FIG. 438 shows the flower of strawberry cultivar ‘11.107-5’.

FIG. 439 shows the plant of strawberry cultivar ‘11.109-2’.

FIG. 440 shows fruit of strawberry cultivar ‘11.109-2’.

FIG. 441 shows the flower of strawberry cultivar ‘11.109-2’.

FIG. 442 shows the plant of strawberry cultivar ‘11.109-3’.

FIG. 443 shows fruit of strawberry cultivar ‘11.109-3’.

FIG. 444 shows the flower of strawberry cultivar ‘11.109-3’.

FIG. 445 shows the plant of strawberry cultivar ‘11.110-3’.

FIG. 446 shows fruit of strawberry cultivar ‘11.110-3’.

FIG. 447 shows the flower of strawberry cultivar ‘11.110-3’.

FIG. 448 shows the plant of strawberry cultivar ‘11.113-6’.

FIG. 449 shows fruit of strawberry cultivar ‘11.113-6’.

FIG. 450 shows the flower of strawberry cultivar ‘11.113-6’.

FIG. 451 shows the plant of strawberry cultivar ‘11.115-3’.

FIG. 452 shows fruit of strawberry cultivar ‘11.115-3’.

FIG. 453 shows the flower of strawberry cultivar ‘11.115-3’.

FIG. 454 shows the plant of strawberry cultivar ‘11.116-1’.

FIG. 455 shows fruit of strawberry cultivar ‘11.116-1’.

FIG. 456 shows the flower of strawberry cultivar ‘11.116-1’.

FIG. 457 shows the plant of strawberry cultivar ‘11.129-2’.

FIG. 458 shows fruit of strawberry cultivar ‘11.129-2’.

FIG. 459 shows the flower of strawberry cultivar ‘11.129-2’.

FIG. 460 shows the plant of strawberry cultivar ‘11.141-1’.

FIG. 461 shows fruit of strawberry cultivar ‘11.141-1’.

FIG. 462 shows the flower of strawberry cultivar ‘11.141-1’.

FIG. 463 shows the plant of strawberry cultivar ‘11.145-1’.

FIG. 464 shows fruit of strawberry cultivar ‘11.145-1’.

FIG. 465 shows the flower of strawberry cultivar ‘11.145-1’.

FIG. 466 shows the plant of strawberry cultivar ‘11.151-3’.

FIG. 467 shows fruit of strawberry cultivar ‘11.151-3’.

FIG. 468 shows the flower of strawberry cultivar ‘11.151-3’.

FIG. 469 shows the plant of strawberry cultivar ‘11.151-8’.

FIG. 470 shows fruit of strawberry cultivar ‘11.151-8’.

FIG. 471 shows the flower of strawberry cultivar ‘11.151-8’.

FIG. 472 shows the plant of strawberry cultivar ‘11.153-2’.

FIG. 473 shows fruit of strawberry cultivar ‘11.153-2’.

FIG. 474 shows the flower of strawberry cultivar ‘11.153-2’.

FIG. 475 shows the plant of strawberry cultivar ‘11.153-3’.

FIG. 476 shows fruit of strawberry cultivar ‘11.153-3’.

FIG. 477 shows the flower of strawberry cultivar ‘11.153-3’.

FIG. 478 shows the plant of strawberry cultivar ‘11.154-5’.

FIG. 479 shows fruit of strawberry cultivar ‘11.154-5’.

FIG. 480 shows the flower of strawberry cultivar ‘11.154-5’.

FIG. 481 shows the plant of strawberry cultivar ‘11.157-1’.

FIG. 482 shows fruit of strawberry cultivar ‘11.157-1’.

FIG. 483 shows the flower of strawberry cultivar ‘11.157-1’.

FIG. 484 shows the plant of strawberry cultivar ‘11.158-1’.

FIG. 485 shows fruit of strawberry cultivar ‘11.158-1’.

FIG. 486 shows the flower of strawberry cultivar ‘11.158-1’.

FIG. 487 shows the plant of strawberry cultivar ‘11.167-4’.

FIG. 488 shows fruit of strawberry cultivar ‘11.167-4’.

FIG. 489 shows the flower of strawberry cultivar ‘11.167-4’.

FIG. 490 shows the plant of strawberry cultivar ‘11.179-3’.

FIG. 491 shows fruit of strawberry cultivar ‘11.179-3’.

FIG. 492 shows the flower of strawberry cultivar ‘11.179-3’.

FIG. 493 shows the plant of strawberry cultivar ‘11.180-1’.

FIG. 494 shows fruit of strawberry cultivar ‘11.180-1’.

FIG. 495 shows the flower of strawberry cultivar ‘11.180-1’.

FIG. 496 shows the plant of strawberry cultivar ‘11.181-2’.

FIG. 497 shows fruit of strawberry cultivar ‘11.181-2’.

FIG. 498 shows the flower of strawberry cultivar ‘11.181-2’.

FIG. 499 shows the plant of strawberry cultivar ‘11.190-3’.

FIG. 500 shows fruit of strawberry cultivar ‘11.190-3’.

FIG. 501 shows the flower of strawberry cultivar ‘11.190-3’.

FIG. 502 shows the plant of strawberry cultivar ‘11.195-3’.

FIG. 503 shows fruit of strawberry cultivar ‘11.195-3’.

FIG. 504 shows the flower of strawberry cultivar ‘11.195-3’.

### DETAILED DESCRIPTION OF THE INVENTION

The following description is based on observations taken of plants and fruits of the new and distinctive strawberry cultivars of the present invention. The new and distinctive strawberry cultivars of the present invention are characterized and distinguished by their resistance to Verticillium, Phytophthora, Fusarium, and/or Macrophomina, by their yield, by their appearance, by their size, and by their firmness.

In the tables below, as applicable, “Vert” refers to resistance score for Verticillium resistance; “Phy” refers to resistance score for Phytophthora; “Fus” refers to resistance score for Fusarium resistance; and “Mac” refers to resistance score for Macrophomina resistance. For a given resistance score, a score of 5 represents resistance. Accordingly, a plant with a score of 5 is resistant. A plant with a score of 5 is more resistant than a plant with a score of 4. A plant with a score of 4 is more resistant than a plant with a score of 3. A plant with a score of 3 is more resistant than a plant with a score of 2. A plant with a score of 2 is more resistant than a plant with a score of 1. A score of 1 represents susceptibility. Accordingly, a plant with a score of 1 is susceptible. “Yield” refers to seasonal yield in grams per plant. “App” refers to commercial appearance score on a scale of 1 to 5, with a 5 representing the best score. “Size” refers to seasonal fruit size in grams/fruit. “Firm” refers to firmness, as measured by a Hunter force gauge rating in #/square inch.

### Cultivar ‘01.80-602’

In certain aspects, the present invention relates to a new and distinctive day-neutral type strawberry cultivar designated as ‘01.80-602’. Cultivar ‘01.80-602’ is typical of day-neutral strawberry cultivars and produces fruit regardless of day length when treated appropriately in arid, subtropical climates.

The strawberry cultivar ‘01.80-602’ originated from a cross between two unreleased germplasm accessions ‘97.86-1’ and ‘98.78-601’. The cultivar has been asexually reproduced by runners. The properties of this cultivar were found to be transmissible by such asexual reproduction. The cultivar is stable and reproduces true to type in successive generations of asexual reproduction.

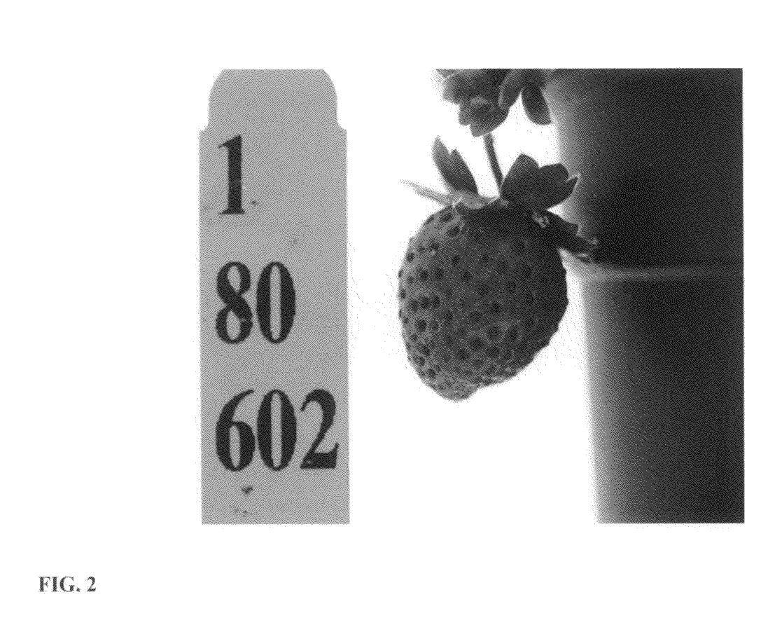

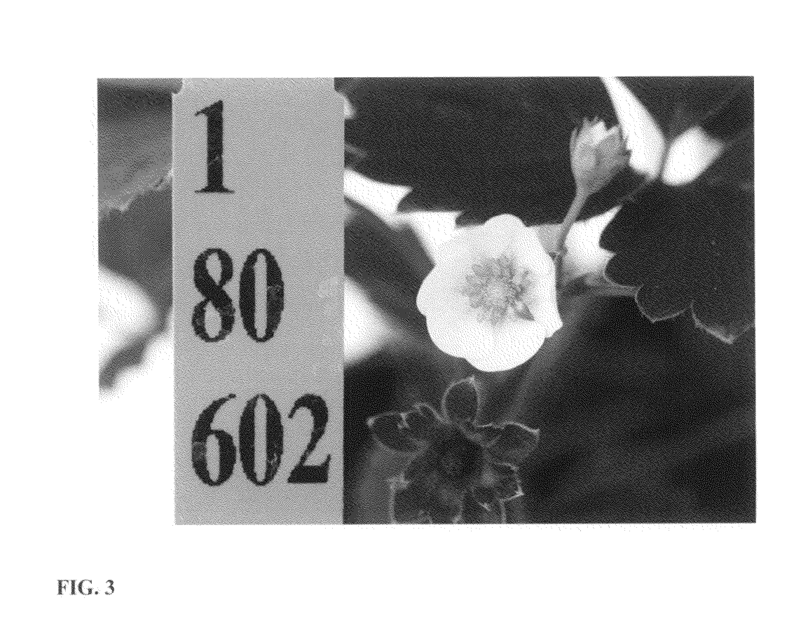

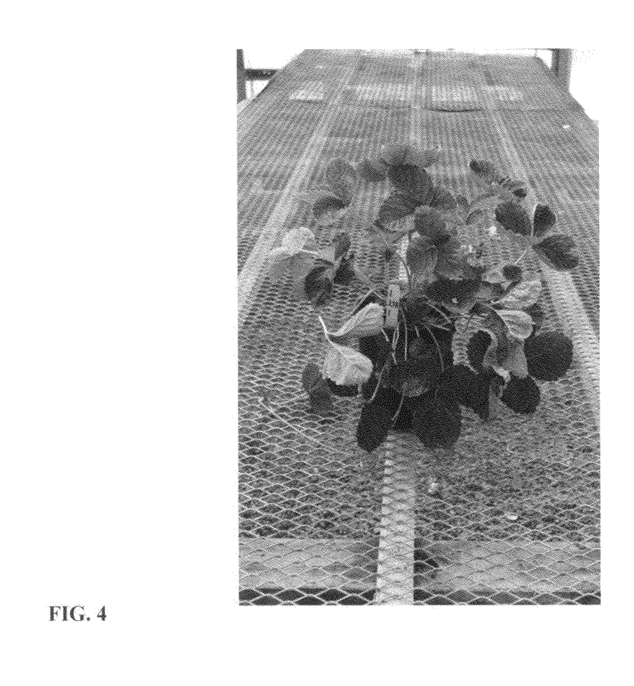

Cultivar ‘01.80-602’ has distinct disease/pathogen resistance, yield, appearance, size, and firmness. The plant of cultivar ‘01.80-602’ is characterized and distinguished in FIG. 1, the fruit of cultivar ‘01.80-602’ is characterized and distinguished in FIG. 2, and the flower of cultivar ‘01.80-602’ is characterized and distinguished in FIG. 3.

The following detailed description of cultivar ‘01.80-602’ is based on observations taken of plants and fruits (see FIGS. 1 and 2).

Certain characteristics of cultivar ‘01.80-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 1 provides data that characterizes the disease/pathogen resistance, yield, appearance, size, and firmness of cultivar ‘01.80-602’.

TABLE 1

| Cultivar | Vert | Phy | Yield | App | Size | Firm |
|---|---|---|---|---|---|---|
| 01.80-602 | 4.5 | 3.5 | 2094 | 3.3 | 29.9 | 10.2 |

### Cultivar ‘01.138-1’

In certain aspects, the present invention relates to a new and distinctive day-neutral type strawberry cultivar designated as ‘01.138-1’. Cultivar ‘01.138-1’ is typical of day-neutral strawberry cultivars and produces fruit regardless of day length when treated appropriately in arid, subtropical climates.

The strawberry cultivar ‘01.138-1’ originated from a cross between two unreleased germplasm accessions ‘97.110-5’ and ‘98.86-1’. The cultivar has been asexually reproduced by runners. The properties of this cultivar were found to be transmissible by such asexual reproduction. The cultivar is stable and reproduces true to type in successive generations of asexual reproduction.

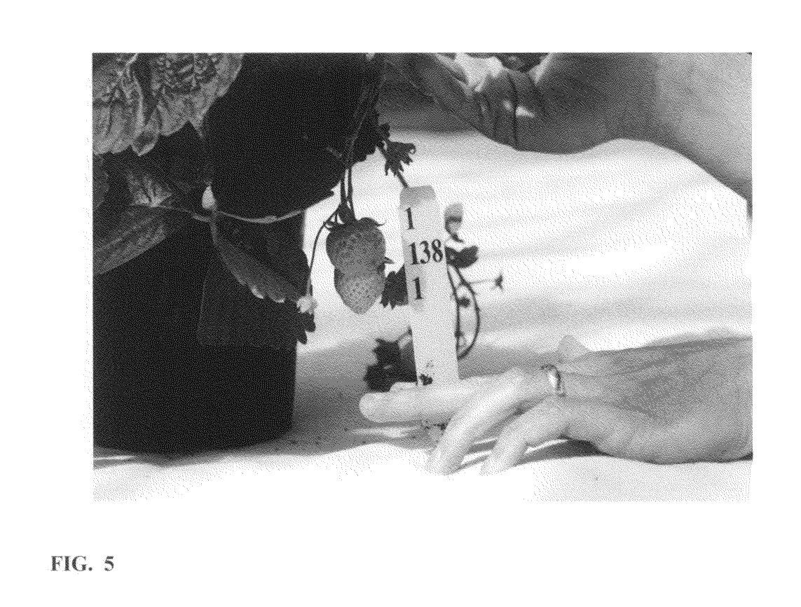

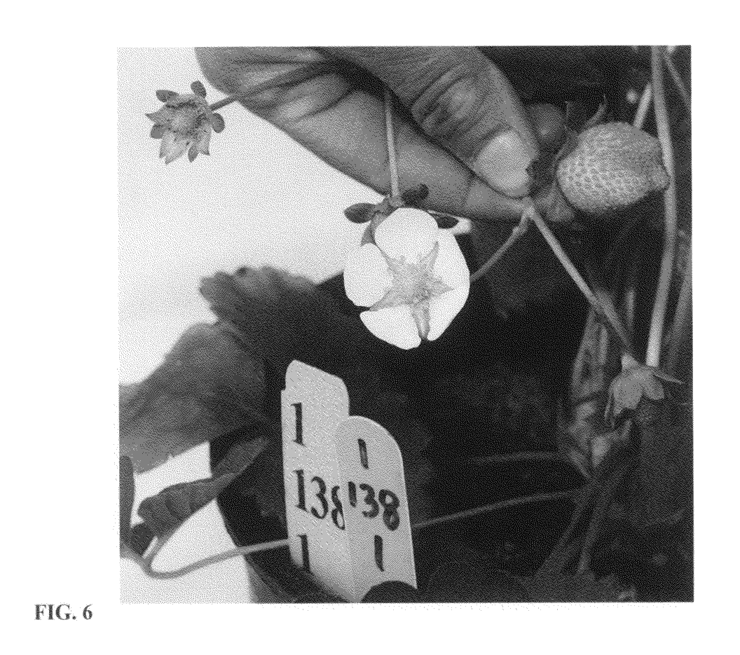

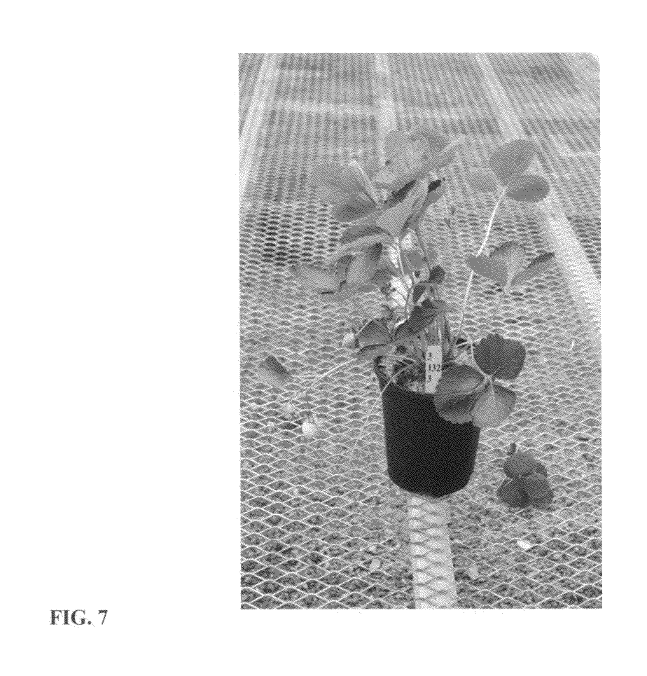

Cultivar ‘01.138-1’ has distinct disease/pathogen resistance, yield, appearance, size, and firmness. The plant of cultivar ‘01.138-1’ is characterized and distinguished in FIG. 4, the fruit of cultivar ‘01.138-1’ is characterized and distinguished in FIG. 5, and the flower of cultivar ‘01.138-1’ is characterized and distinguished in FIG. 6.

The following detailed description of cultivar ‘01.138-1’ is based on observations taken of plants and fruits (see FIGS. 4 and 5).

Certain characteristics of cultivar ‘01.138-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 2 provides data that characterizes the disease/pathogen resistance, yield, appearance, size, and firmness of cultivar ‘01.138-1’.

TABLE 2

| Cultivar | Vert | Phy | Yield | App | Size | Firm |
|---|---|---|---|---|---|---|
| 01.138-1 | 4.0 | 4.0 | 1530 | 3.2 | 28.9 | 10.5 |

### Cultivar ‘03.132-3’

In certain aspects, the present invention relates to a new and distinctive short-day type strawberry cultivar designated as ‘03.132-3’. Cultivar ‘03.132-3’ is typical of short-day strawberry cultivars and produces fruit over an extended period when treated appropriately in arid, subtropical climates.

The strawberry cultivar ‘03.132-3’ originated from a cross between two unreleased germplasm accessions ‘99.88-7’ and ‘98.153-3’. The cultivar has been asexually reproduced by runners. The properties of this cultivar were found to be transmissible by such asexual reproduction. The cultivar is stable and reproduces true to type in successive generations of asexual reproduction.

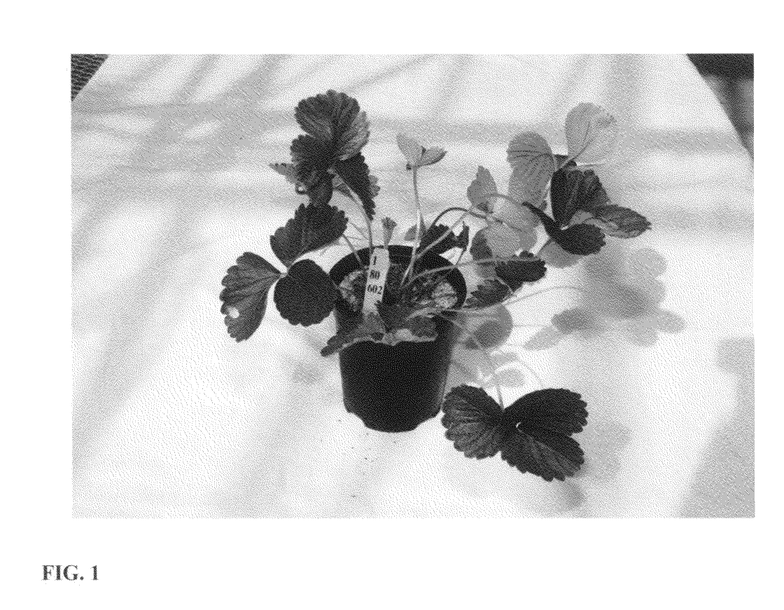

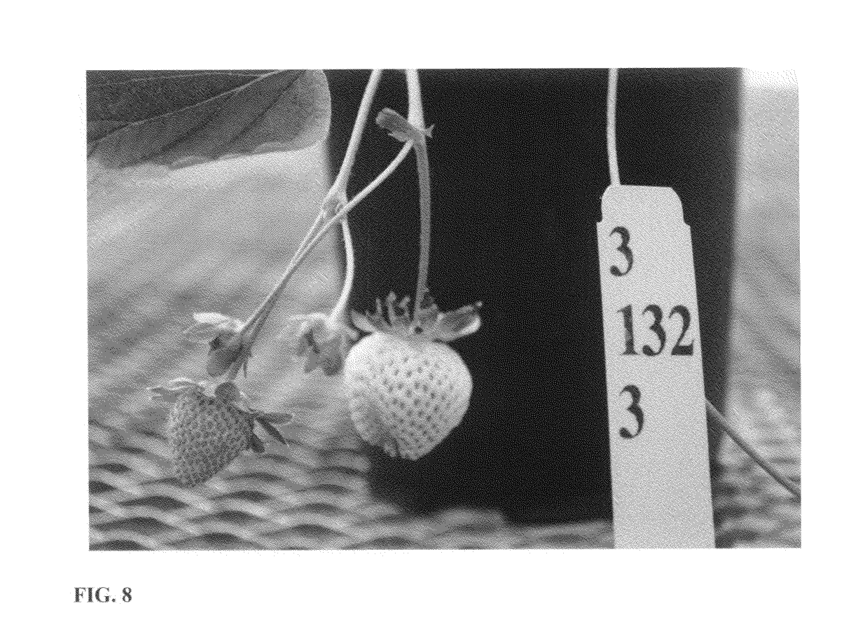

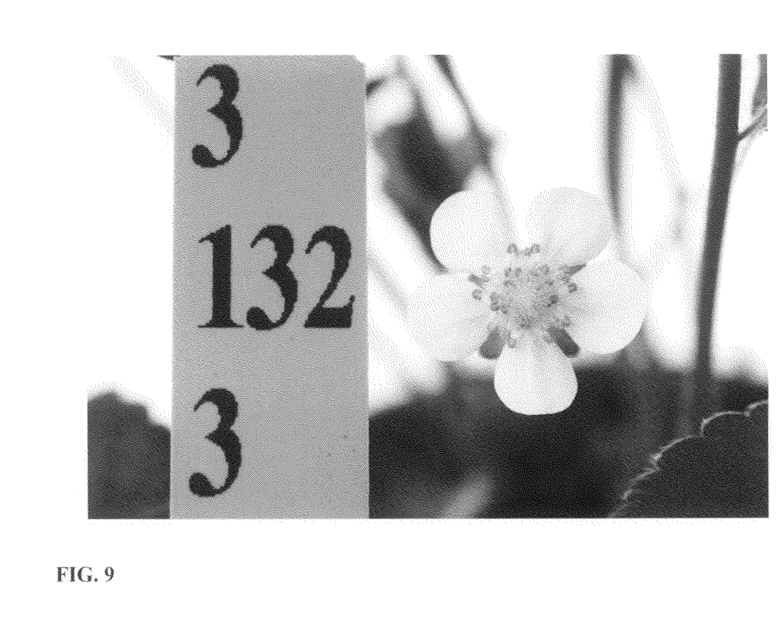

Cultivar ‘03.132-3’ has distinct disease/pathogen resistance, yield, appearance, size, and firmness. The plant of cultivar ‘03.132-3’ is characterized and distinguished in FIG. 7, the fruit of cultivar ‘03.132-3’ is characterized and distinguished in FIG. 8, and the flower of cultivar ‘03.132-3’ is characterized and distinguished in FIG. 9.

The following detailed description of cultivar ‘03.132-3’ is based on observations taken of plants and fruits (see FIGS. 7 and 8).

Certain characteristics of cultivar ‘03.132-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 3 provides data that characterizes the disease/pathogen resistance, yield, appearance, size, and firmness of cultivar ‘03.132-3’.

TABLE 3

| Cultivar | Vert | Yield | App | Size | Firm |
|---|---|---|---|---|---|
| 03.132-3 | 3.1 | 2595 | 3.7 | 34.1 | 10.2 |

### Cultivar ‘04.9-5’

In certain aspects, the present invention relates to a new and distinctive short-day type strawberry cultivar designated as ‘04.9-5’. Cultivar ‘04.9-5’ is typical of short-day strawberry cultivars and produces fruit over an extended period when treated appropriately in arid, subtropical climates.

The strawberry cultivar ‘04.9-5’ originated from a cross between cultivar ‘Palomar’ (U.S. Plant Pat. No. 19,472) and unreleased germplasm accession ‘01.24-602’. The cultivar has been asexually reproduced by runners. The properties of this cultivar were found to be transmissible by such asexual reproduction. The cultivar is stable and reproduces true to type in successive generations of asexual reproduction.

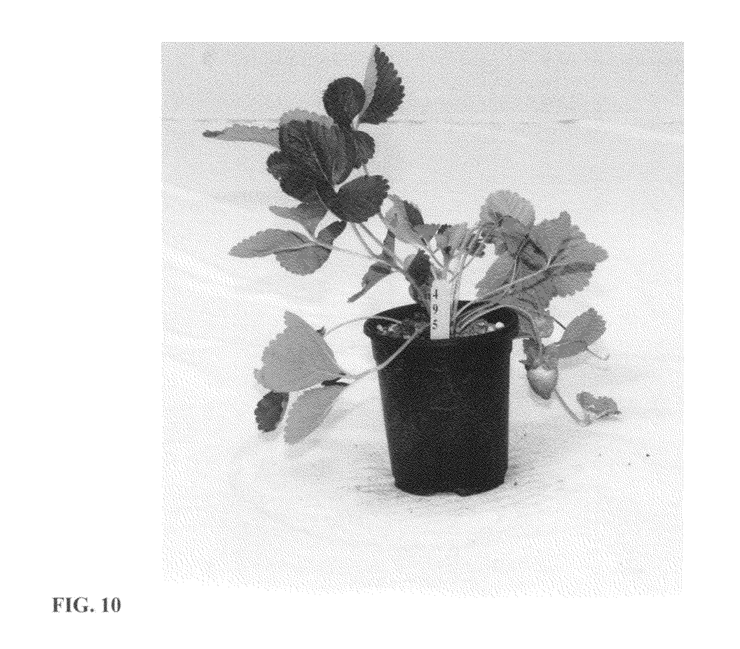

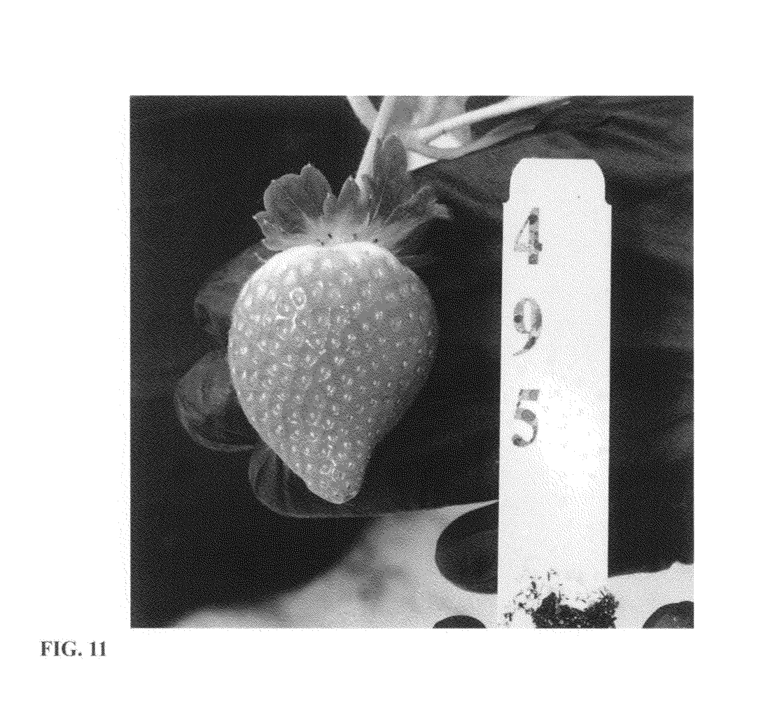

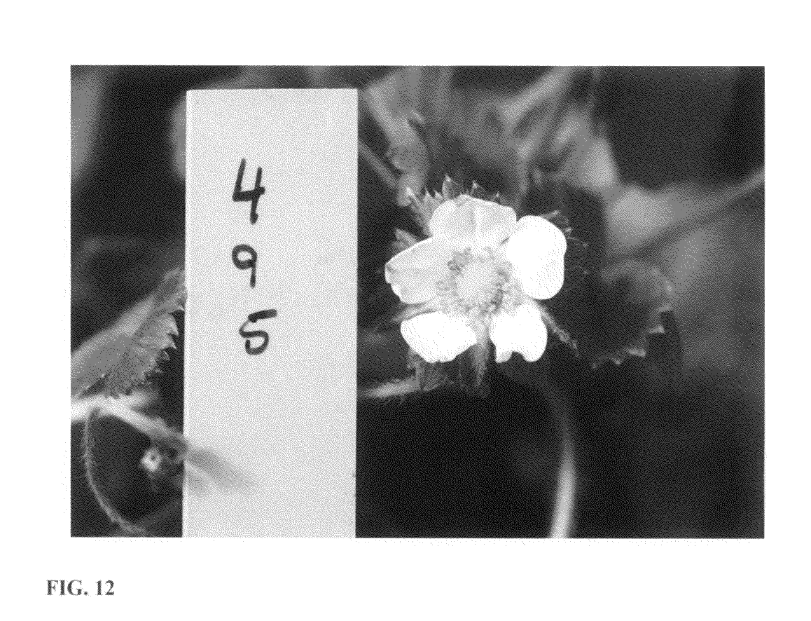

Cultivar ‘04.9-5’ has distinct disease/pathogen resistance, yield, appearance, size, and firmness. The plant of cultivar ‘04.9-5’ is characterized and distinguished in FIG. 10, the fruit of cultivar ‘04.9-5’ is characterized and distinguished in FIG. 11, and the flower of cultivar ‘04.9-5’ is characterized and distinguished in FIG. 12.

The following detailed description of cultivar ‘04.9-5’ is based on observations taken of plants and fruits (see FIGS. 10 and 11).

Certain characteristics of cultivar ‘04.9-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 4 provides data that characterizes the disease/pathogen resistance, yield, appearance, size, and firmness of cultivar ‘04.9-5’.

TABLE 4

| Cultivar | Vert | Phy | Yield | App | Size | Firm |
|---|---|---|---|---|---|---|
| 04.9-5 | 3.3 | 3.5 | 3070 | 3.8 | 33.3 | 10.5 |

### Cultivar ‘04.18-4’

In certain aspects, the present invention relates to a new and distinctive short-day type strawberry cultivar designated as ‘04.18-4’. Cultivar ‘04.18-4’ is typical of short-day strawberry cultivars and produces fruit over an extended period when treated appropriately in arid, subtropical climates.

The strawberry cultivar ‘04.18-4’ originated from a cross between cultivar ‘Palomar’ (U.S. Plant Pat. No. 19,472) and unreleased germplasm accession ‘01.80-602’. The cultivar has been asexually reproduced by runners. The properties of this cultivar were found to be transmissible by such asexual reproduction. The cultivar is stable and reproduces true to type in successive generations of asexual reproduction.

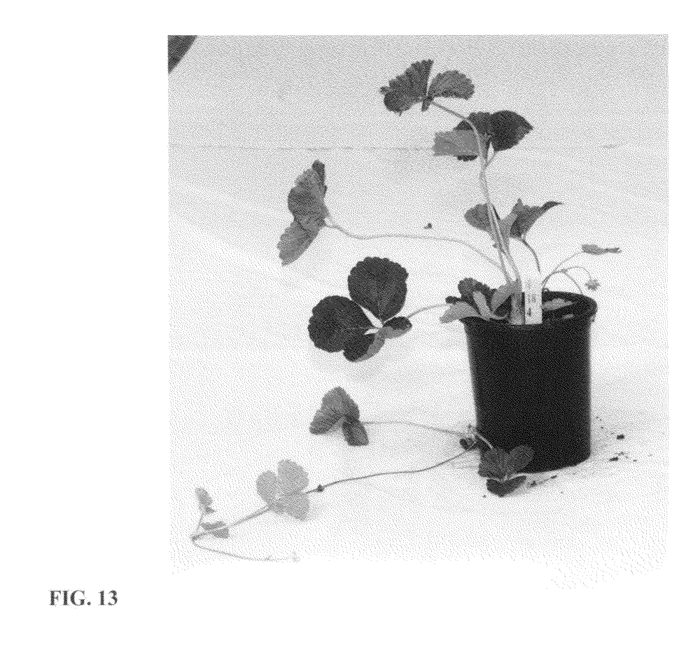

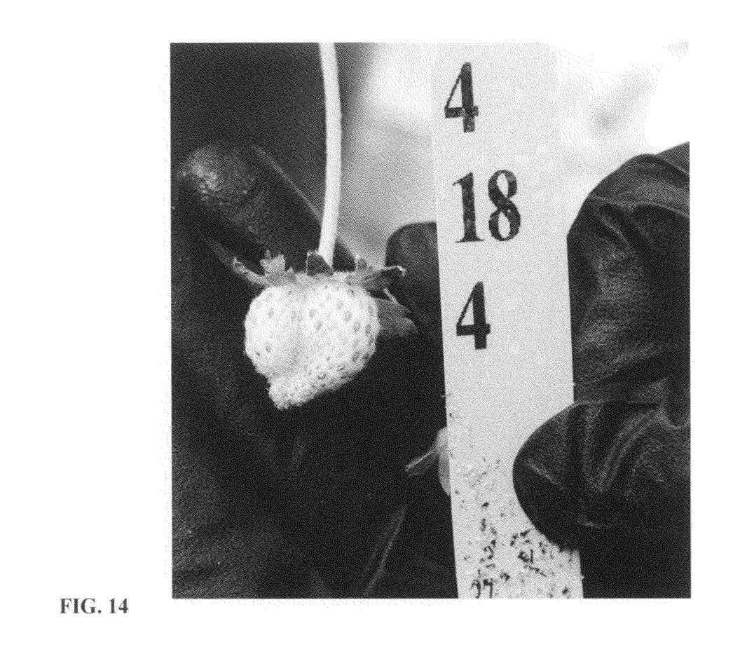

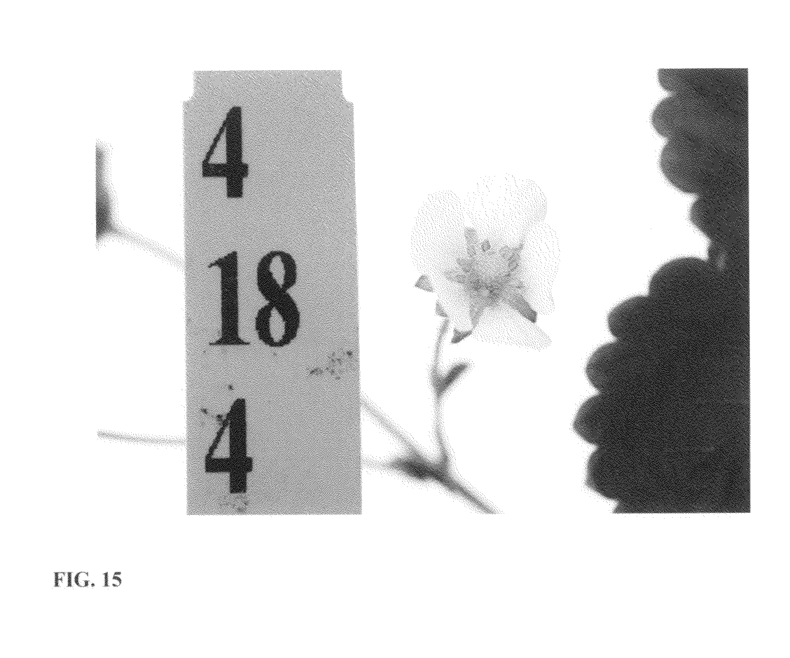

Cultivar ‘04.18-4’ has distinct disease/pathogen resistance, yield, appearance, size, and firmness. The plant of cultivar ‘04.18-4’ is characterized and distinguished in FIG. 13, the fruit of cultivar ‘04.18-4’ is characterized and distinguished in FIG. 14, and the flower of cultivar ‘04.18-4’ is characterized and distinguished in FIG. 15.

The following detailed description of cultivar ‘04.18-4’ is based on observations taken of plants and fruits (see FIGS. 13 and 14).

Certain characteristics of cultivar ‘04.18-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 5 provides data that characterizes the disease/pathogen resistance, yield, appearance, size, and firmness of cultivar ‘04.18-4’.

TABLE 5

| Cultivar | Vert | Phy | Yield | App | Size | Firm |
|---|---|---|---|---|---|---|
| 04.18-4 | 4.6 | 3.3 | 2836 | 3.6 | 38.1 | 9.8 |

### Cultivar ‘04.41-6’

In certain aspects, the present invention relates to a new and distinctive short-day type strawberry cultivar designated as ‘04.41-6’. Cultivar ‘04.41-6’ is typical of short-day strawberry cultivars and produces fruit over an extended period when treated appropriately in arid, subtropical climates.

The strawberry cultivar ‘04.41-6’ originated from a cross between unreleased germplasm accession ‘00.139-3’ and cultivar ‘Palomar’ (U.S. Plant Pat. No. 19,472). The cultivar has been asexually reproduced by runners. The properties of this cultivar were found to be transmissible by such asexual reproduction. The cultivar is stable and reproduces true to type in successive generations of asexual reproduction.

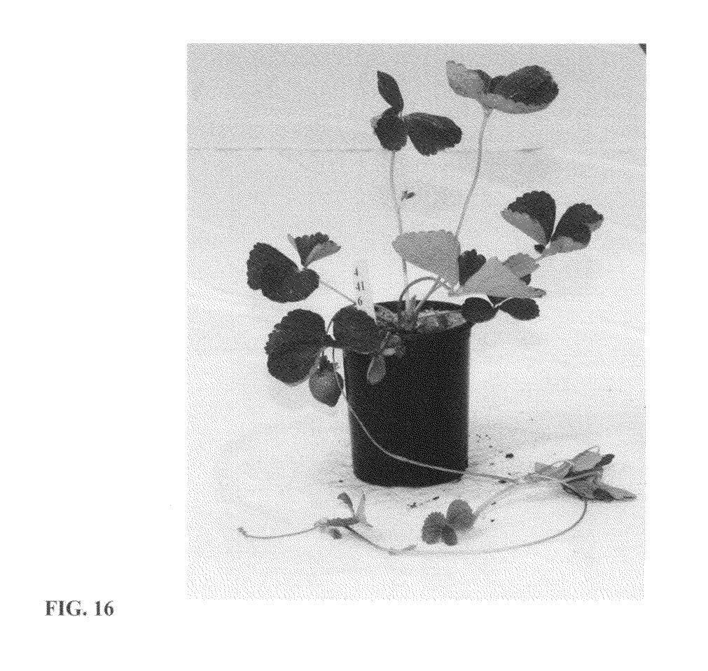

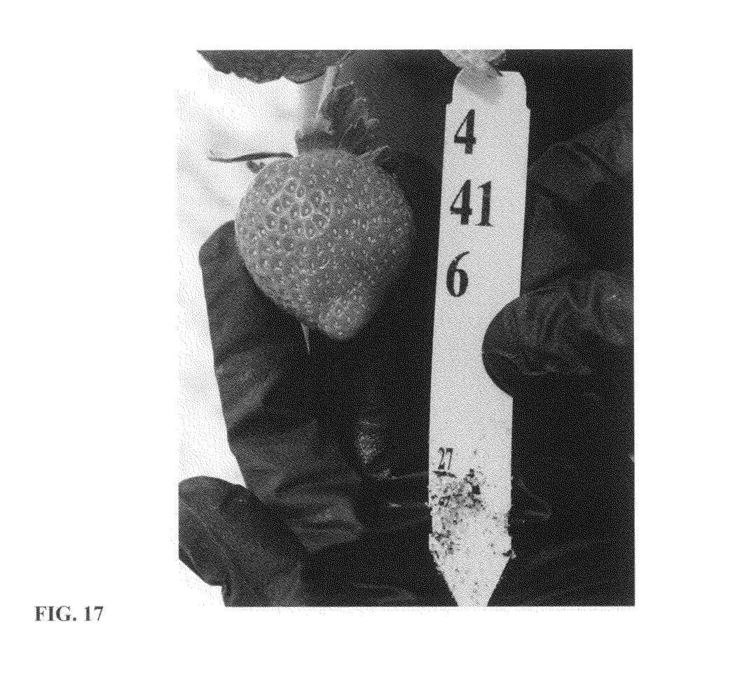

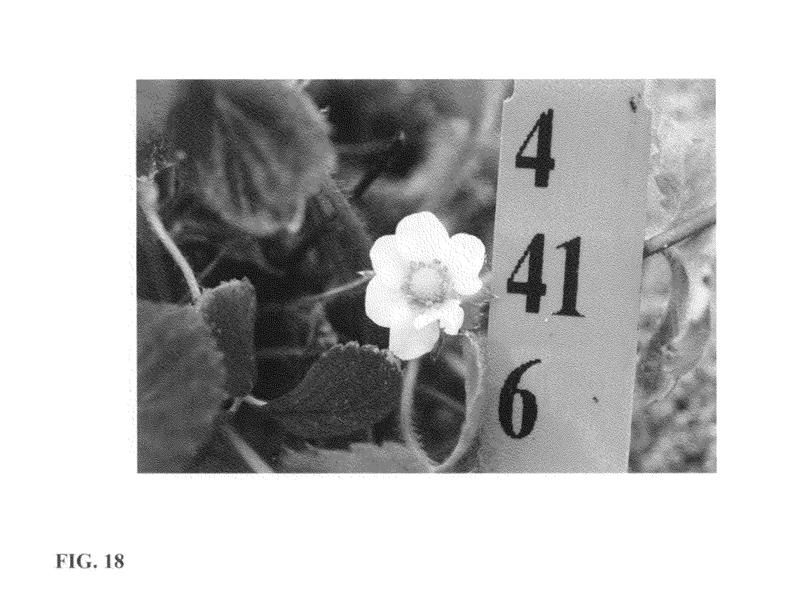

Cultivar ‘04.41-6’ has distinct disease/pathogen resistance, yield, appearance, size, and firmness. The plant of cultivar ‘04.41-6’ is characterized and distinguished in FIG. 16, the fruit of cultivar ‘04.41-6’ is characterized and distinguished in FIG. 17, and the flower of cultivar ‘04.41-6’ is characterized and distinguished in FIG. 18.

The following detailed description of cultivar ‘04.41-6’ is based on observations taken of plants and fruits (see FIGS. 16 and 17).

Certain characteristics of cultivar ‘04.41-6’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 6 provides data that characterizes the disease/pathogen resistance, yield, appearance, size, and firmness of cultivar ‘04.41-6’.

TABLE 6

| Cultivar | Vert | Phy | Yield | App | Size | Firm |
|---|---|---|---|---|---|---|
| 04.41-6 | 3.4 | 2.8 | 2814 | 3.9 | 33.2 | 10.8 |

### Cultivar ‘04.41-7’

In certain aspects, the present invention relates to a new and distinctive short-day type strawberry cultivar designated as ‘04.41-7’. Cultivar ‘04.41-7’ is typical of short-day strawberry cultivars and produces fruit over an extended period when treated appropriately in arid, subtropical climates.

The strawberry cultivar ‘04.41-7’ originated from a cross between unreleased germplasm accession ‘00.139-3’ and cultivar ‘Palomar’ (U.S. Plant Pat. No. 19,472). The cultivar has been asexually reproduced by runners. The properties of this cultivar were found to be transmissible by such asexual reproduction. The cultivar is stable and reproduces true to type in successive generations of asexual reproduction.

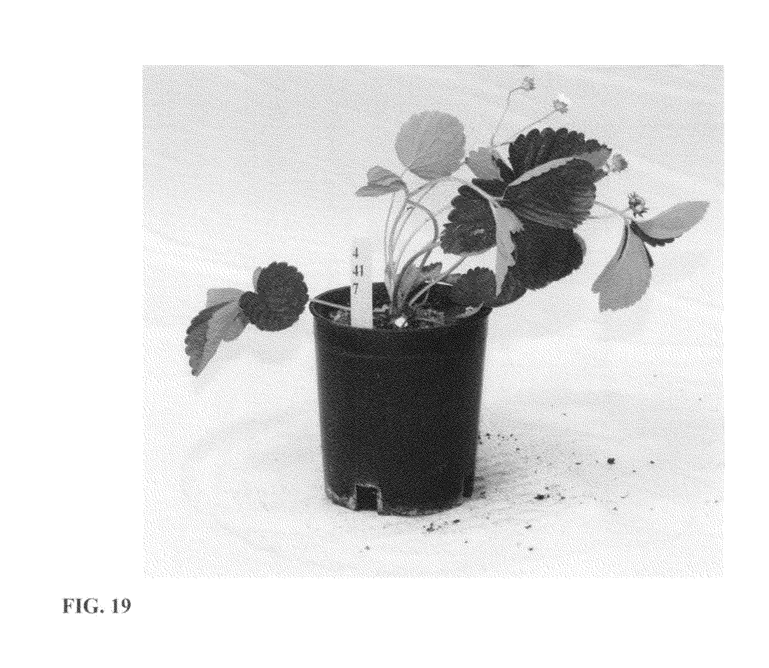

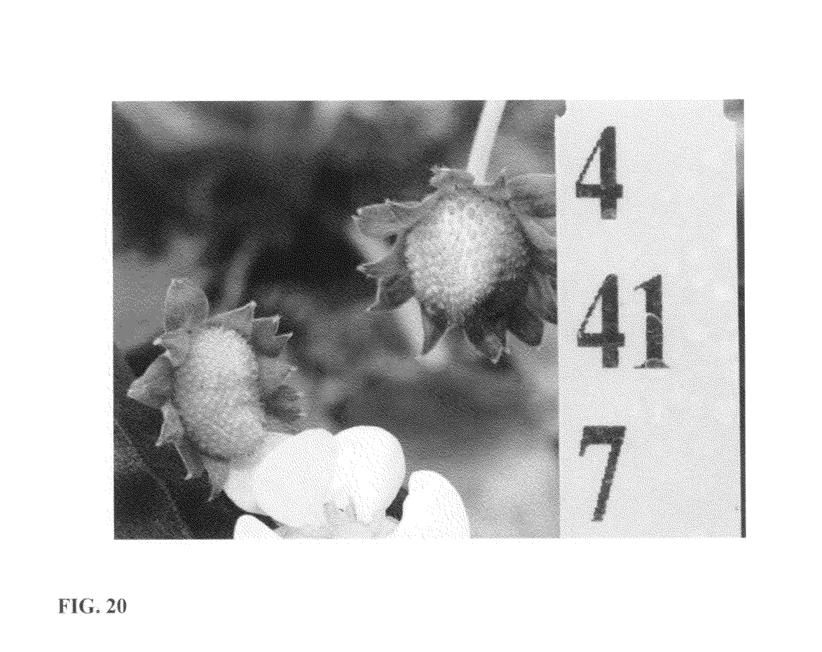

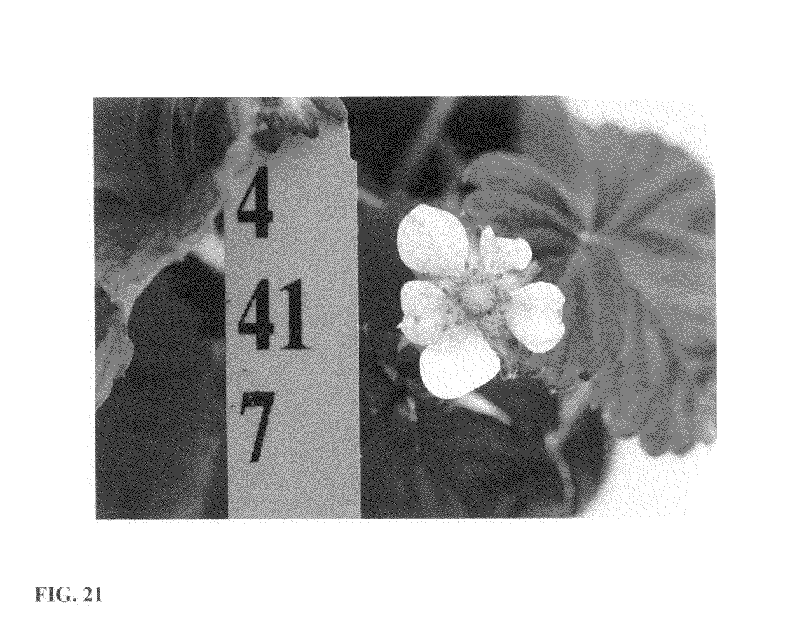

Cultivar ‘04.41-7’ has distinct disease/pathogen resistance, yield, appearance, size, and firmness. The plant of cultivar ‘04.41-7’ is characterized and distinguished in FIG. 19, the fruit of cultivar ‘04.41-7’ is characterized and distinguished in FIG. 20, and the flower of cultivar ‘04.41-7’ is characterized and distinguished in FIG. 21.

The following detailed description of cultivar ‘04.41-7’ is based on observations taken of plants and fruits (see FIGS. 19 and 20).

Certain characteristics of cultivar ‘04.41-7’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 7 provides data that characterizes the disease/pathogen resistance, yield, appearance, size, and firmness of cultivar ‘04.41-7’.

TABLE 7

| Cultivar | Vert | Phy | Yield | App | Size | Firm |
|---|---|---|---|---|---|---|
| 04.41-7 | 4.1 | 3.4 | 2315 | 3.6 | 33.4 | 9.5 |

### Cultivar ‘04.76-4’

In certain aspects, the present invention relates to a new and distinctive short-day type strawberry cultivar designated as ‘04.76-4’. Cultivar ‘04.76-4’ is typical of short-day strawberry cultivars and produces fruit over an extended period when treated appropriately in arid, subtropical climates.

The strawberry Cultivar ‘04.76-4’ originated from a cross between two unreleased germplasm accessions ‘01.57-601’ and ‘01.80-602’. The cultivar has been asexually reproduced by runners. The properties of this cultivar were found to be transmissible by such asexual reproduction. The cultivar is stable and reproduces true to type in successive generations of asexual reproduction.

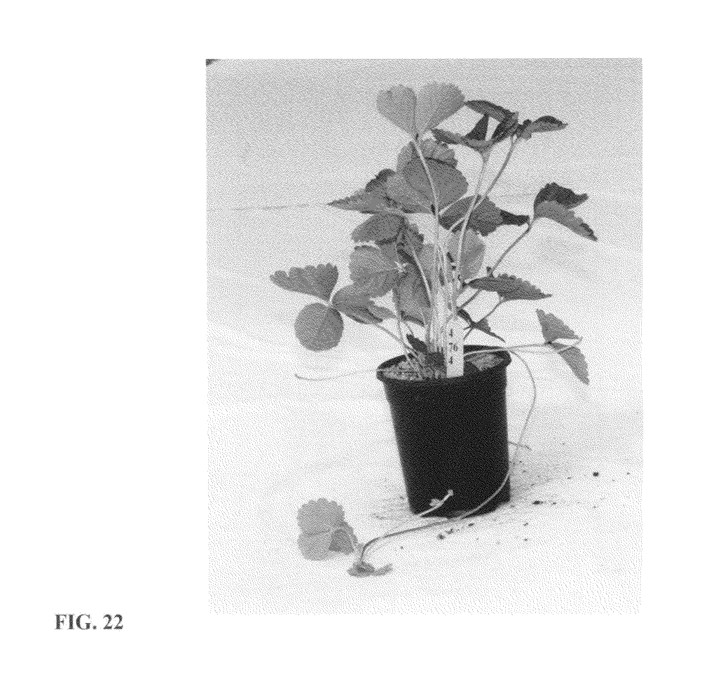

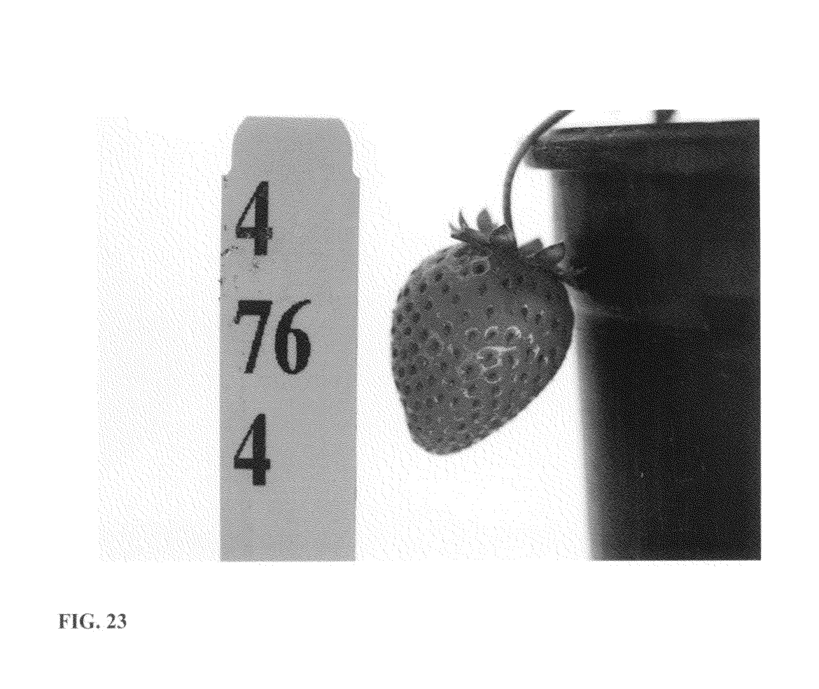

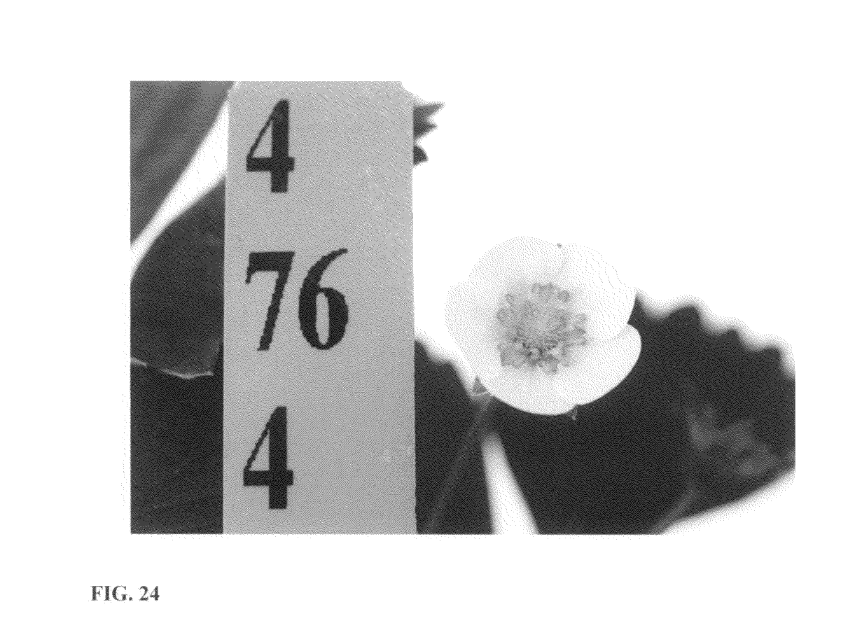

Cultivar ‘04.76-4’ has distinct disease/pathogen resistance, yield, appearance, size, and firmness. The plant of cultivar ‘04.76-4’ is characterized and distinguished in FIG. 22, the fruit of cultivar ‘04.76-4’ is characterized and distinguished in FIG. 23, and the flower of cultivar ‘04.76-4’ is characterized and distinguished in FIG. 24.

The following detailed description of cultivar ‘04.76-4’ is based on observations taken of plants and fruits (see FIGS. 22 and 23).

Certain characteristics of cultivar ‘04.76-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 8 provides data that characterizes the disease/pathogen resistance, yield, appearance, size, and firmness of cultivar ‘04.76-4’.

TABLE 8

| Cultivar | Vert | Phy | Fus | Yield | App | Size | Firm |
|---|---|---|---|---|---|---|---|
| 04.76-4 | 4.9 | 4.9 | 4.6 | 2359 | 4.1 | 34.4 | 14.0 |

### Cultivar ‘04.77-2’

In certain aspects, the present invention relates to a new and distinctive short-day type strawberry cultivar designated as ‘04.77-2’. Cultivar ‘04.77-2’ is typical of short-day strawberry cultivars and produces fruit over an extended period when treated appropriately in arid, subtropical climates.

The strawberry cultivar ‘04.77-2’ originated from a cross between two unreleased germplasm accessions ‘01.61-602’ and ‘01.80-602’. The cultivar has been asexually reproduced by runners. The properties of this cultivar were found to be transmissible by such asexual reproduction. The cultivar is stable and reproduces true to type in successive generations of asexual reproduction.

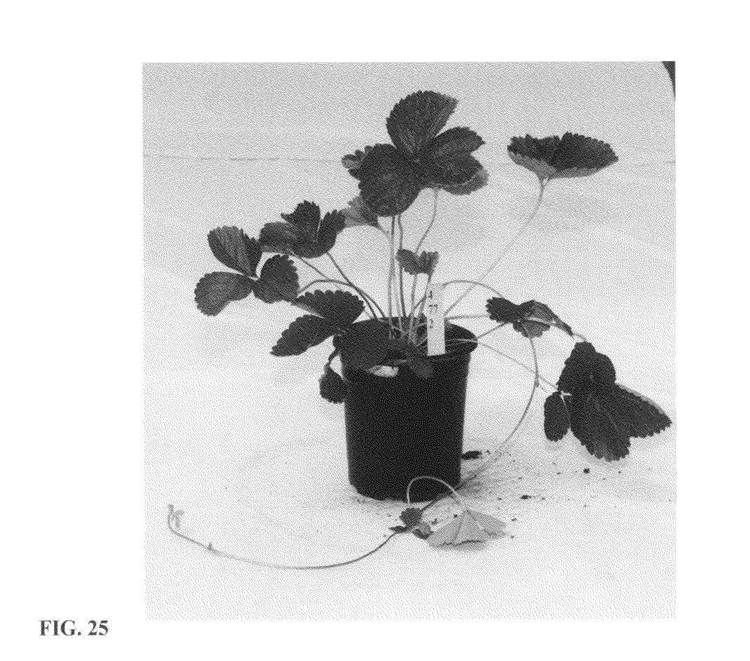

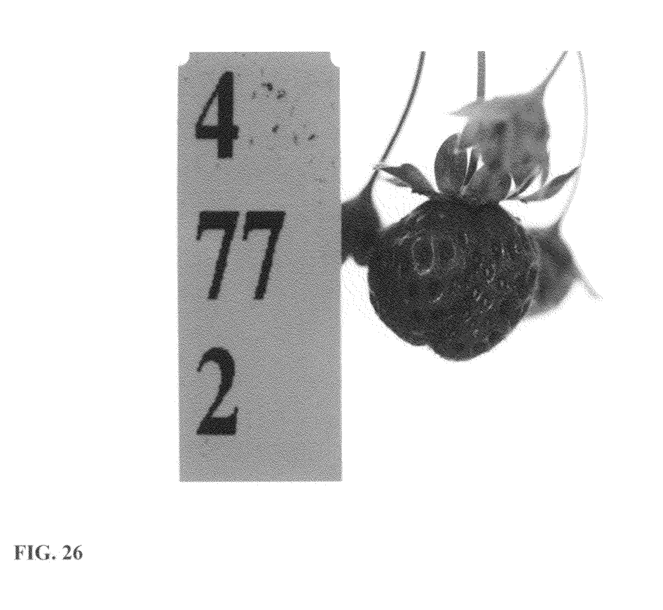

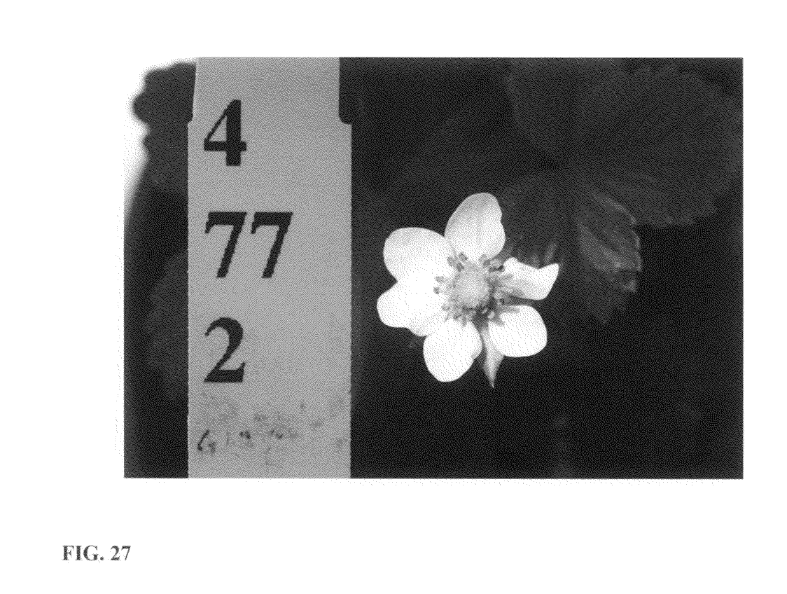

Cultivar ‘04.77-2’ has distinct disease/pathogen resistance, yield, appearance, size, and firmness. The plant of cultivar ‘04.77-2’ is characterized and distinguished in FIG. 25, the fruit of cultivar ‘04.77-2’ is characterized and distinguished in FIG. 26, and the flower of cultivar ‘04.77-2’ is characterized and distinguished in FIG. 27.

The following detailed description of cultivar ‘04.77-2’ is based on observations taken of plants and fruits (see FIGS. 25 and 26).

Certain characteristics of cultivar ‘04.77-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 9 provides data that characterizes the disease/pathogen resistance, yield, appearance, size, and firmness of cultivar ‘04.77-2’.

TABLE 9

| Cultivar | Vert | Phy | Yield | App | Size | Firm |
|---|---|---|---|---|---|---|
| 04.77-2 | 3.9 | 4.8 | 2206 | 3.4 | 33.8 | 10.6 |

### Cultivar ‘05.92-11’

In certain aspects, the present invention relates to a new and distinctive day-neutral type strawberry cultivar designated as ‘05.92-11’. Cultivar ‘05.92-11’ is typical of day-neutral strawberry cultivars and produces fruit regardless of day length when treated appropriately in arid, subtropical climates.

The strawberry cultivar ‘05.92-11’ originated from a cross between cultivar ‘Palomar’ (U.S. Plant Pat. No. 19,472) and cultivar ‘Monterey’ (U.S. Plant Pat. No. 19,767). The cultivar has been asexually reproduced by runners. The properties of this cultivar were found to be transmissible by such asexual reproduction. The cultivar is stable and reproduces true to type in successive generations of asexual reproduction.

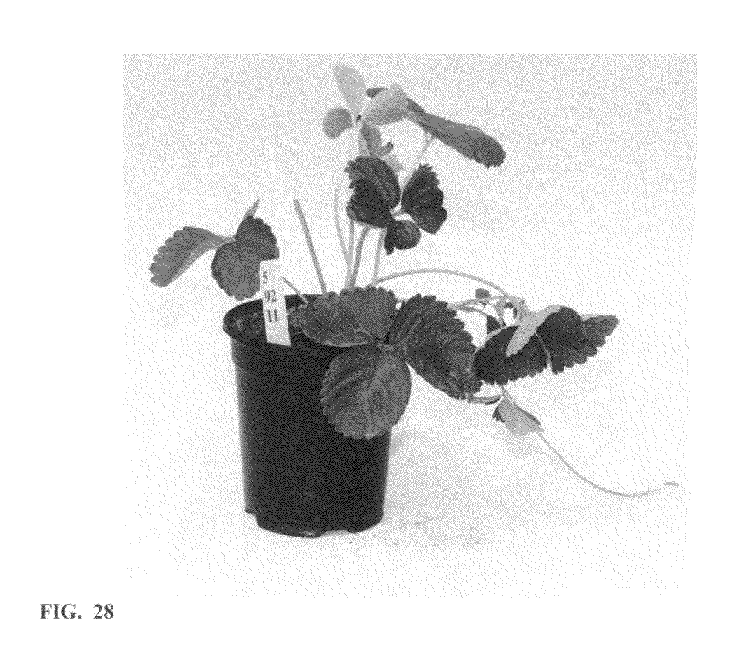

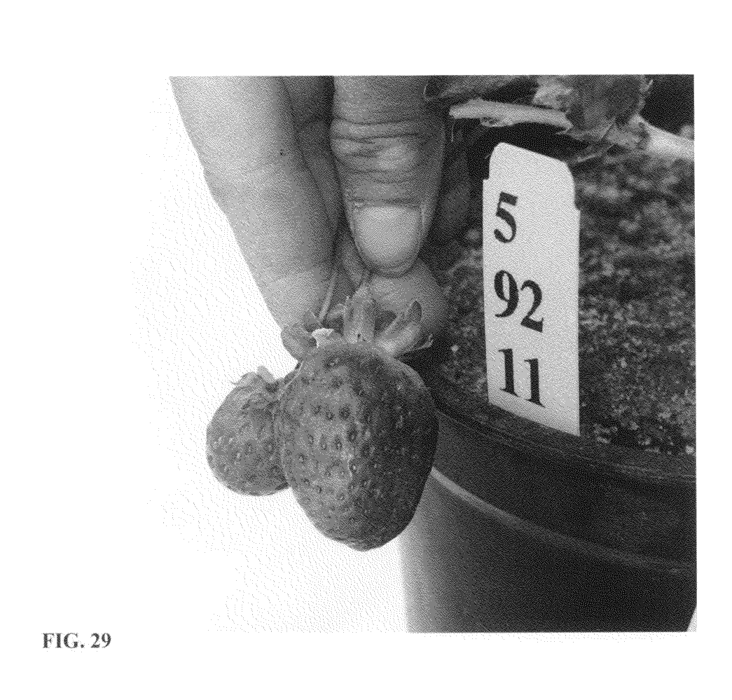

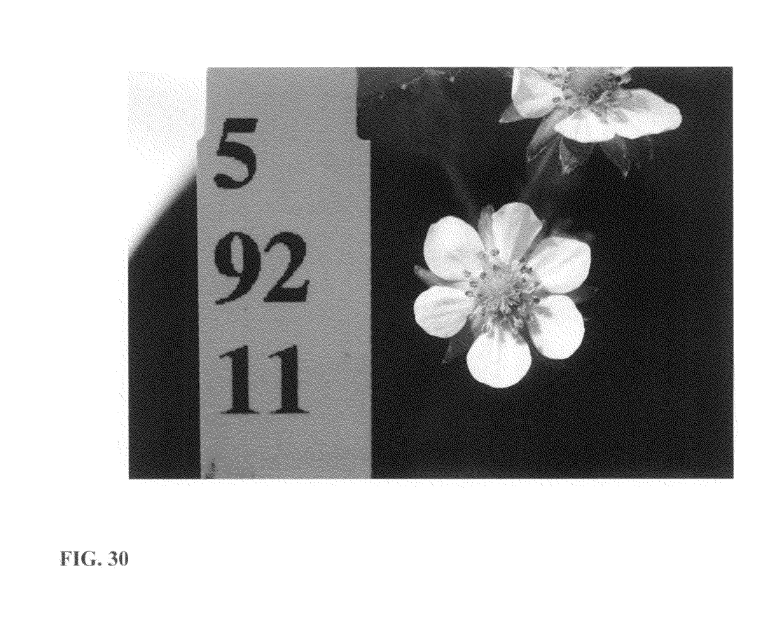

Cultivar ‘05.92-11’ has distinct disease/pathogen resistance, yield, appearance, size, and firmness. The plant of cultivar ‘05.92-11’ is characterized and distinguished in FIG. 28, the fruit of cultivar ‘05.92-11’ is characterized and distinguished in FIG. 29, and the flower of cultivar ‘05.92-11’ is characterized and distinguished in FIG. 30.

The following detailed description of cultivar ‘05.92-11’ is based on observations taken of plants and fruits (see FIGS. 28 and 29).

Certain characteristics of cultivar ‘05.92-11’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 10 provides data that characterizes the disease/pathogen resistance, yield, appearance, size, and firmness of cultivar ‘05.92-11’.

TABLE 10

| Cultivar | Vert | Phy | Yield | App | Size | Firm |
|---|---|---|---|---|---|---|
| 05.92-11 | 3.4 | 4.1 | 2475 | 3.4 | 35.0 | 10.1 |

### Cultivar ‘05.95-4’

In certain aspects, the present invention relates to a new and distinctive short-day type strawberry cultivar designated as ‘05.95-4’. Cultivar ‘05.95-4’ is typical of short-day strawberry cultivars and produces fruit over an extended period when treated appropriately in arid, subtropical climates.

The strawberry cultivar ‘05.95-4’ originated from a cross between cultivar ‘Palomar’ (U.S. Plant Pat. No. 19,472) and unreleased germplasm accession ‘01-138-1’. The cultivar has been asexually reproduced by runners. The properties of this cultivar were found to be transmissible by such asexual reproduction. The cultivar is stable and reproduces true to type in successive generations of asexual reproduction.

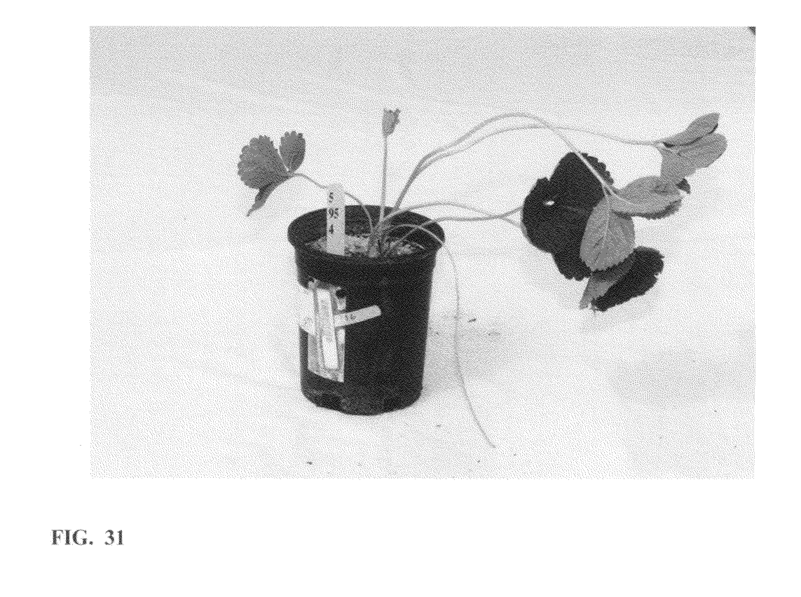

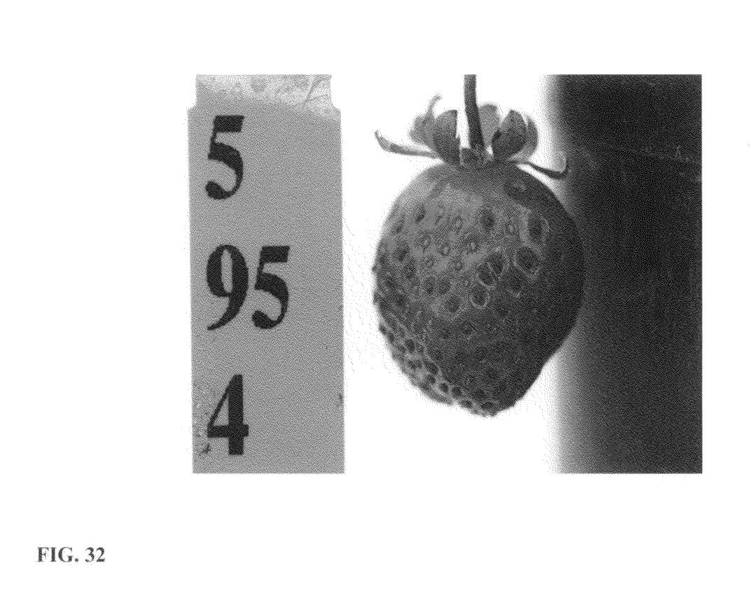

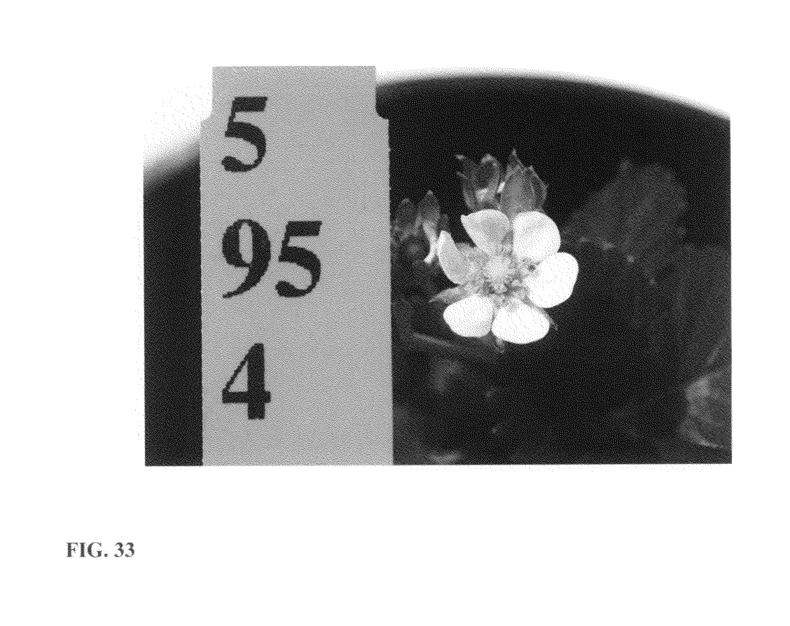

Cultivar ‘05.95-4’ has distinct disease/pathogen resistance, yield, appearance, size, and firmness. The plant of cultivar ‘05.95-4’ is characterized and distinguished in FIG. 31, the fruit of cultivar ‘05.95-4’ is characterized and distinguished in FIG. 32, and the flower of cultivar ‘05.95-4’ is characterized and distinguished in FIG. 33.

The following detailed description of cultivar ‘05.95-4’ is based on observations taken of plants and fruits (see FIGS. 31 and 32).

Certain characteristics of cultivar ‘05.95-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 11 provides data that characterizes the disease/pathogen resistance, yield, appearance, size, and firmness of cultivar ‘05.95-4’.

TABLE 11

| Cultivar | Vert | Phy | Yield | App | Size | Firm |
|---|---|---|---|---|---|---|
| 05.95-4 | 2.9 | 4.0 | 1871 | 3.4 | 39.1 | 9.9 |

### Cultivar ‘05.99-6’

In certain aspects, the present invention relates to a new and distinctive day-neutral type strawberry cultivar designated as ‘05.99-6’. Cultivar ‘05.99-6’ is typical of day-neutral strawberry cultivars and produces fruit regardless of day length when treated appropriately in arid, subtropical climates.

The strawberry cultivar ‘05.99-6’ originated from a cross between unreleased germplasm accession ‘01-138-1’ and cultivar ‘San Andreas’ (U.S. Plant Pat. No. 19,975). The cultivar has been asexually reproduced by runners. The properties of this cultivar were found to be transmissible by such asexual reproduction. The cultivar is stable and reproduces true to type in successive generations of asexual reproduction.

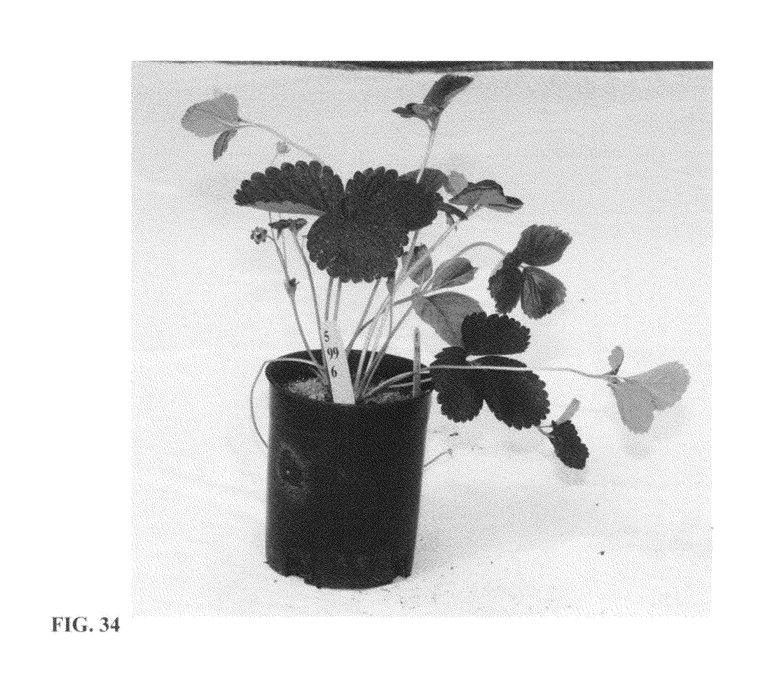

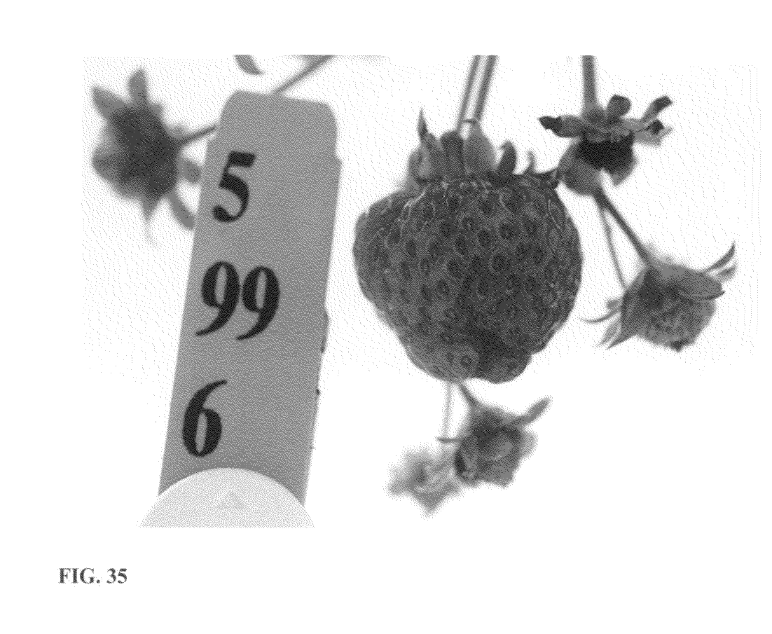

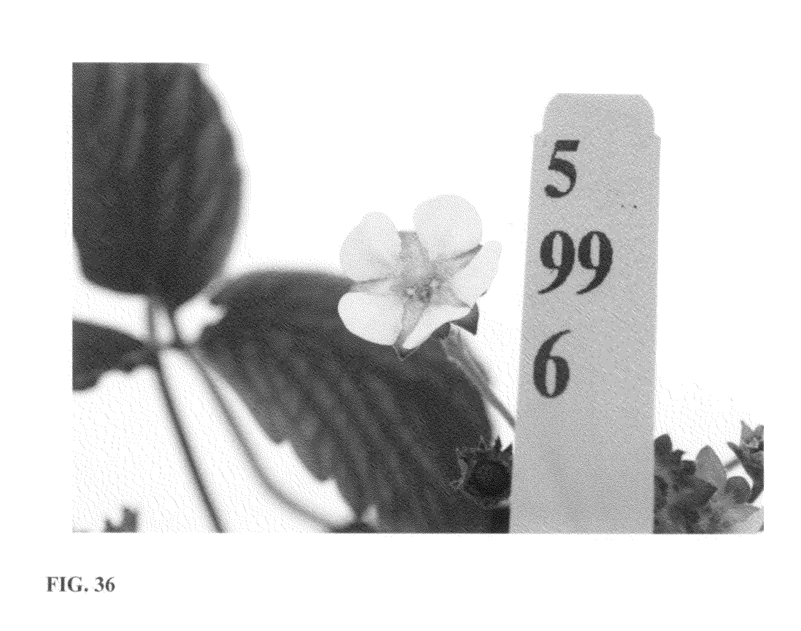

Cultivar ‘05.99-6’ has distinct disease/pathogen resistance, yield, appearance, size, and firmness. The plant of cultivar ‘05.99-6’ is characterized and distinguished in FIG. 34, the fruit of cultivar ‘05.99-6’ is characterized and distinguished in FIG. 35, and the flower of cultivar ‘05.99-6’ is characterized and distinguished in FIG. 36.

The following detailed description of cultivar ‘05.99-6’ is based on observations taken of plants and fruits (see FIGS. 34 and 35).

Certain characteristics of cultivar ‘05.99-6’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 12 provides data that characterizes the disease/pathogen resistance, yield, appearance, size, and firmness of cultivar ‘05.99-6’.

TABLE 12

| Cultivar | Vert | Phy | Fus | Yield | App | Size | Firm |
|---|---|---|---|---|---|---|---|
| 05.99-6 | 4.6 | 3.6 | 4.9 | 2468 | 3.7 | 39.4 | 12.3 |

### Cultivar ‘05.107-2’

In certain aspects, the present invention relates to a new and distinctive short-day type strawberry cultivar designated as ‘05.107-2’. Cultivar ‘05.107-2’ is typical of short-day strawberry cultivars and produces fruit over an extended period when treated appropriately in arid, subtropical climates.

The strawberry cultivar ‘05.107-2’ originated from a cross between cultivar ‘Monterey’ (U.S. Plant Pat. No. 19,767) and unreleased germplasm accession ‘02.143-3’. The cultivar has been asexually reproduced by runners. The properties of this cultivar were found to be transmissible by such asexual reproduction. The cultivar is stable and reproduces true to type in successive generations of asexual reproduction.

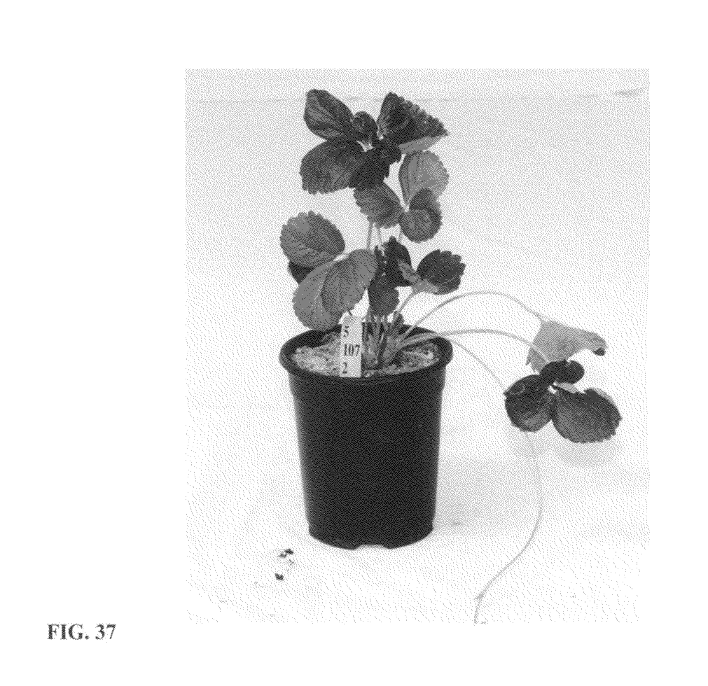

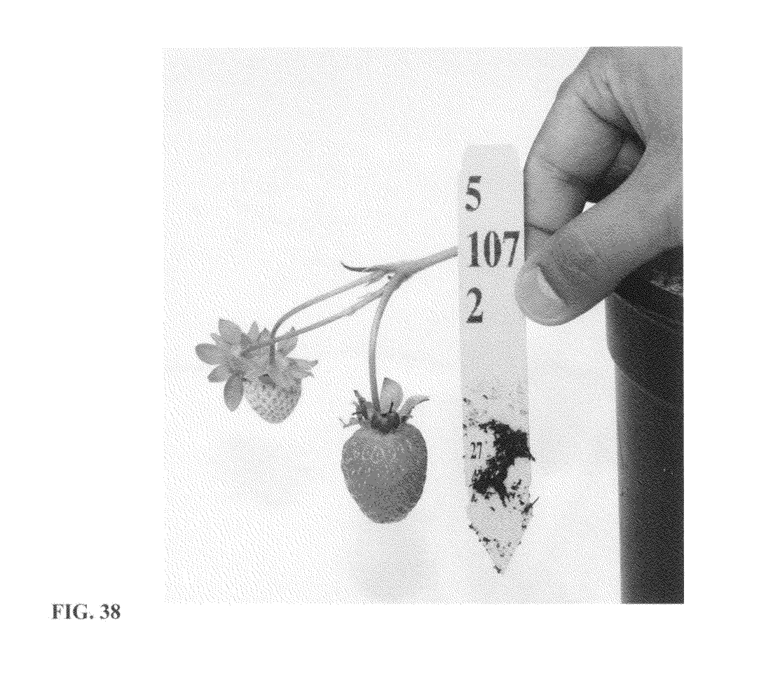

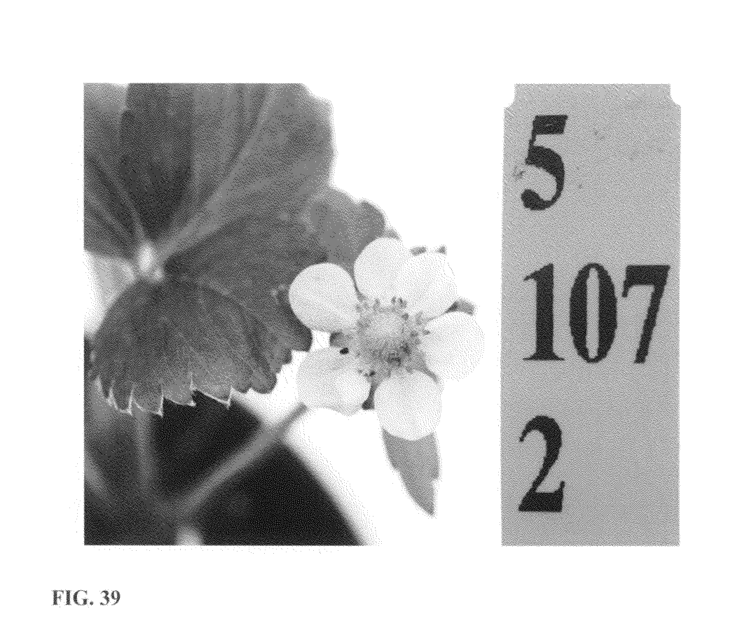

Cultivar ‘05.107-2’ has distinct disease/pathogen resistance, yield, appearance, size, and firmness. The plant of cultivar ‘05.107-2’ is characterized and distinguished in FIG. 37, the fruit of cultivar ‘05.107-2’ is characterized and distinguished in FIG. 38, and the flower of cultivar ‘05.107-2’ is characterized and distinguished in FIG. 39.

The following detailed description of cultivar ‘05.107-2’ is based on observations taken of plants and fruits (see FIGS. 37 and 38).

Certain characteristics of cultivar ‘05.107-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 13 provides data that characterizes the disease/pathogen resistance, yield, appearance, size, and firmness of cultivar ‘05.107-2’.

TABLE 13

| Cultivar | Vert | Phy | Fus | Yield | App | Size | Firm |
|---|---|---|---|---|---|---|---|
| 05.107-2 | 4.6 | 4.8 | 4.6 | 1500 | 3.8 | 34.5 | 11.4 |

### Cultivar ‘05.108-7’

In certain aspects, the present invention relates to a new and distinctive day-neutral type strawberry cultivar designated as ‘05.108-7’. Cultivar ‘05.108-7’ is typical of day-neutral strawberry cultivars and produces fruit regardless of day length when treated appropriately in arid, subtropical climates.

The strawberry cultivar ‘05.108-7’ originated from a cross between two unreleased germplasm accessions ‘01.138-1’ and ‘02.143-3’. The cultivar has been asexually reproduced by runners. The properties of this cultivar were found to be transmissible by such asexual reproduction. The cultivar is stable and reproduces true to type in successive generations of asexual reproduction.

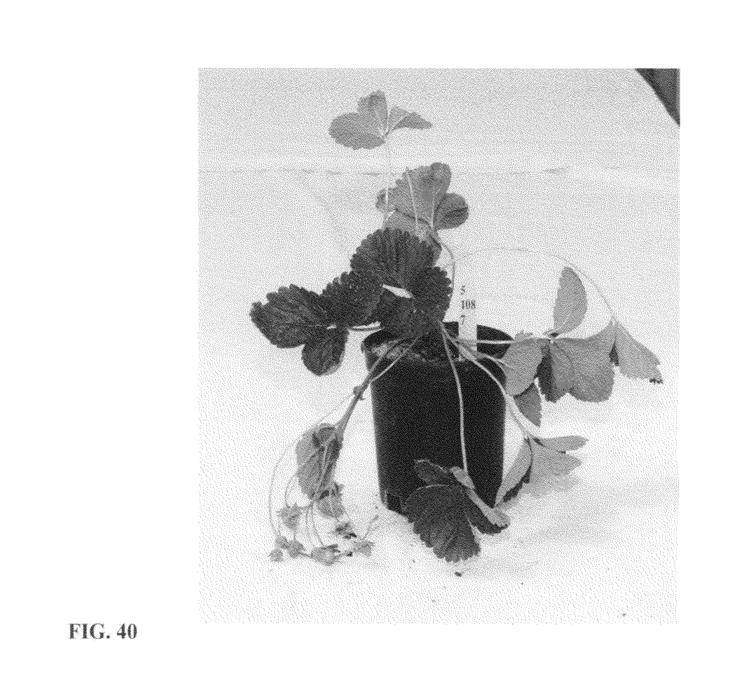

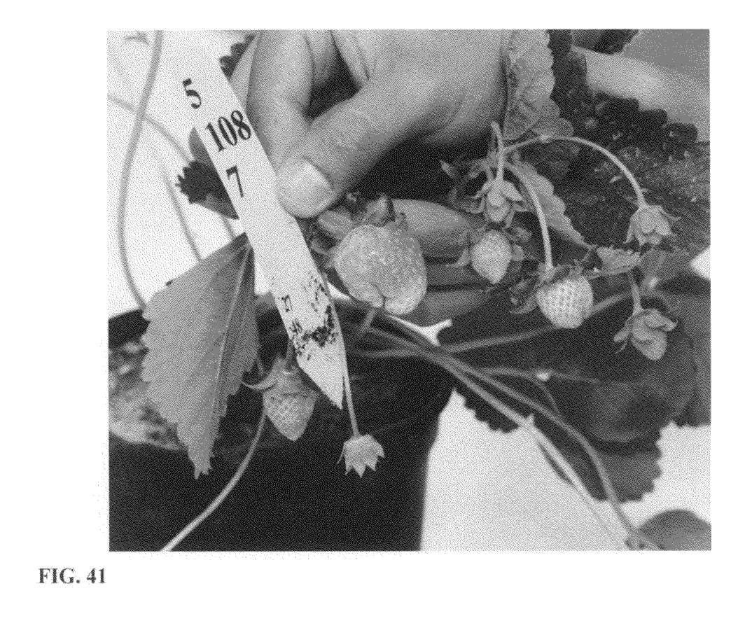

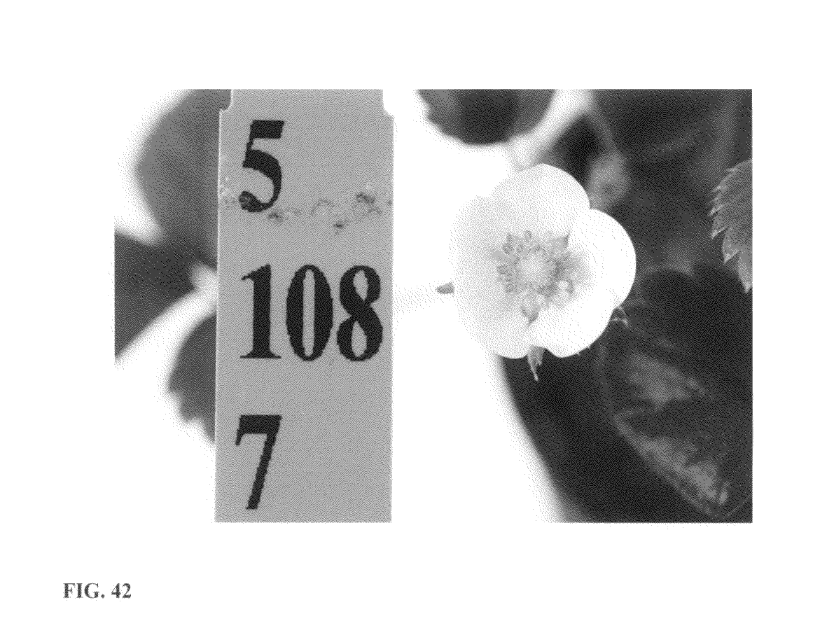

Cultivar ‘05.108-7’ has distinct disease/pathogen resistance, yield, appearance, size, and firmness. The plant of cultivar ‘05.108-7’ is characterized and distinguished in FIG. 40, the fruit of cultivar ‘05.108-7’ is characterized and distinguished in FIG. 41, and the flower of cultivar ‘05.108-7’ is characterized and distinguished in FIG. 42.

The following detailed description of cultivar ‘05.108-7’ is based on observations taken of plants and fruits (see FIGS. 40 and 41).

Certain characteristics of cultivar ‘05.108-7’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 14 provides data that characterizes the disease/pathogen resistance, yield, appearance, size, and firmness of cultivar ‘05.108-7’.

TABLE 14

| Cultivar | Vert | Phy | Fus | Yield | App | Size | Firm |
|---|---|---|---|---|---|---|---|
| 05.108-7 | 4.7 | 3.9 | 5.0 | 2575 | 3.7 | 38.3 | 10.9 |

### Cultivar ‘05.109-2’

In certain aspects, the present invention relates to a new and distinctive short-day type strawberry cultivar designated as ‘05.109-2’. Cultivar ‘05.109-2’ is typical of short-day strawberry cultivars and produces fruit over an extended period when treated appropriately in arid, subtropical climates.

The strawberry cultivar ‘05.109-2’ originated from a cross between cultivar ‘San Andreas’ (U.S. Plant Pat. No. 19,975) and unreleased germplasm accession ‘02.143-3’. The cultivar has been asexually reproduced by runners. The properties of this cultivar were found to be transmissible by such asexual reproduction. The cultivar is stable and reproduces true to type in successive generations of asexual reproduction.

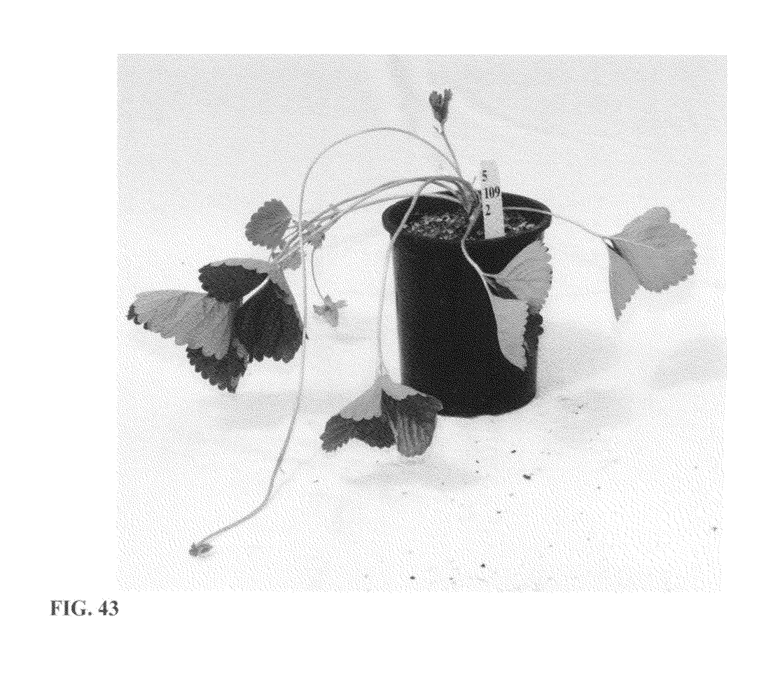

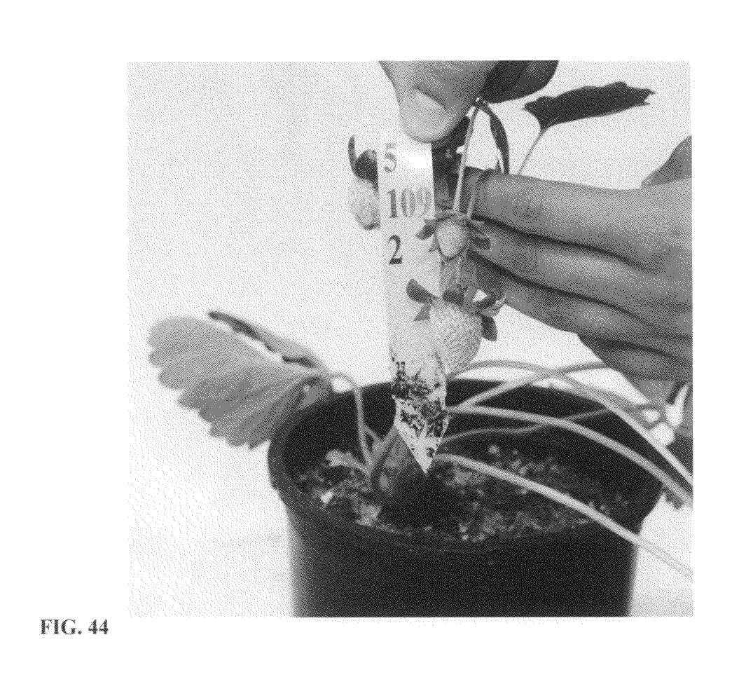

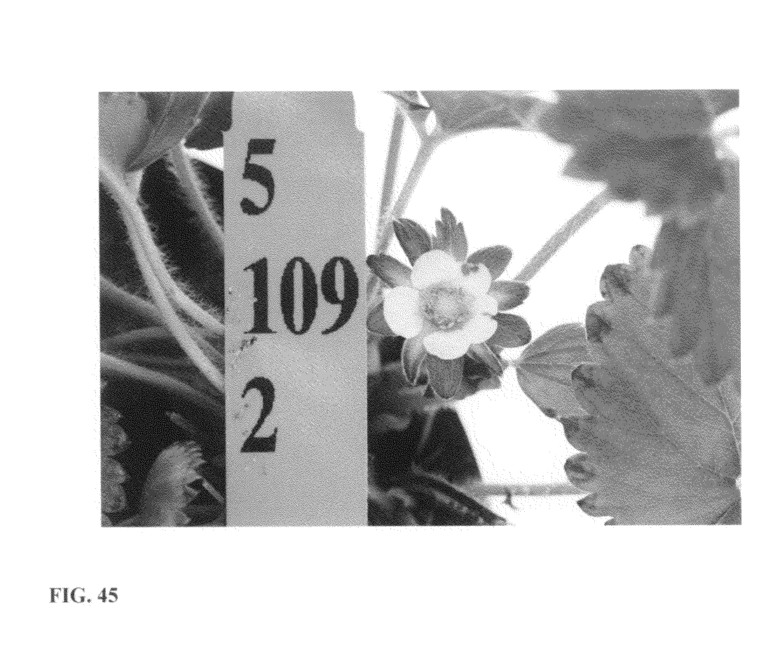

Cultivar ‘05.109-2’ has distinct disease/pathogen resistance, yield, appearance, size, and firmness. The plant of cultivar ‘05.109-2’ is characterized and distinguished in FIG. 43, the fruit of cultivar ‘05.109-2’ is characterized and distinguished in FIG. 44, and the flower of cultivar ‘05.109-2’ is characterized and distinguished in FIG. 45.

The following detailed description of cultivar ‘05.109-2’ is based on observations taken of plants and fruits (see FIGS. 43 and 44).

Certain characteristics of cultivar ‘05.109-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 15 provides data that characterizes the disease/pathogen resistance, yield, appearance, size, and firmness of cultivar ‘05.109-2’.

TABLE 15

| Cultivar | Vert | Phy | Yield | App | Size | Firm |
|---|---|---|---|---|---|---|
| 05.109-2 | 5.0 | 4.5 | 2856 | 3.7 | 35.9 | 12.1 |

### Cultivar ‘05.117-5’

In certain aspects, the present invention relates to a new and distinctive short-day type strawberry cultivar designated as ‘05.117-5’. Cultivar ‘05.117-5’ is typical of short-day strawberry cultivars and produces fruit over an extended period when treated appropriately in arid, subtropical climates.

The strawberry cultivar ‘05.117-5’ originated from a cross between two unreleased germplasm accessions ‘02.93-6’ and ‘00.57-613’. The cultivar has been asexually reproduced by runners. The properties of this cultivar were found to be transmissible by such asexual reproduction. The cultivar is stable and reproduces true to type in successive generations of asexual reproduction.

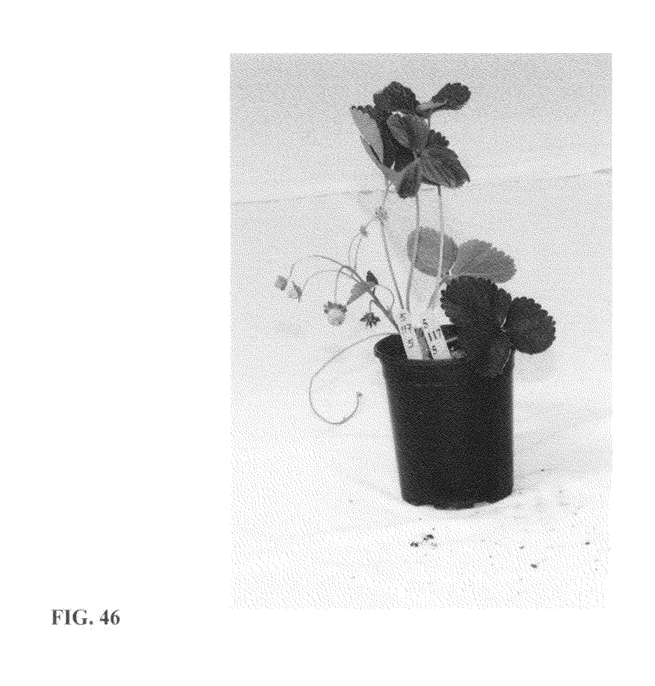

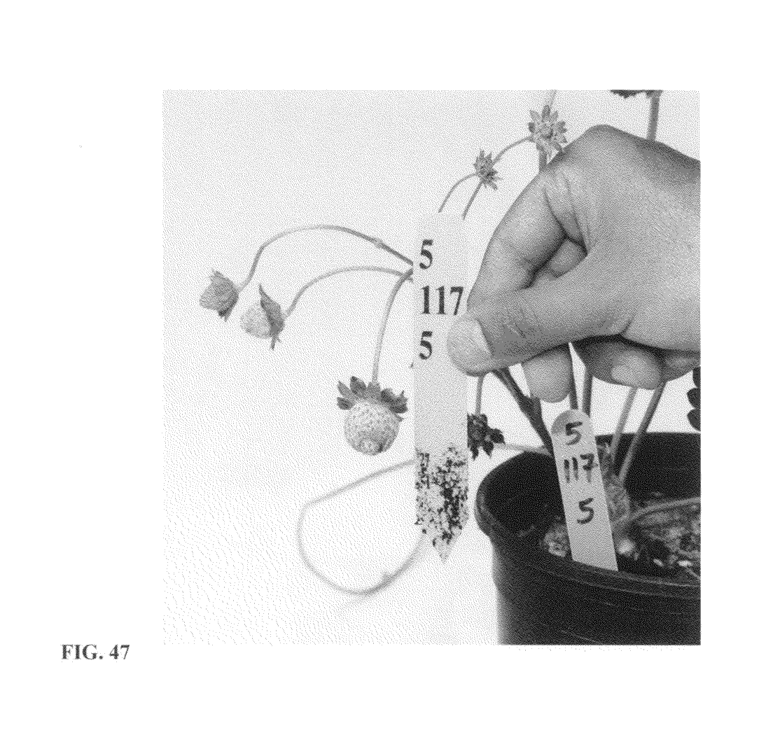

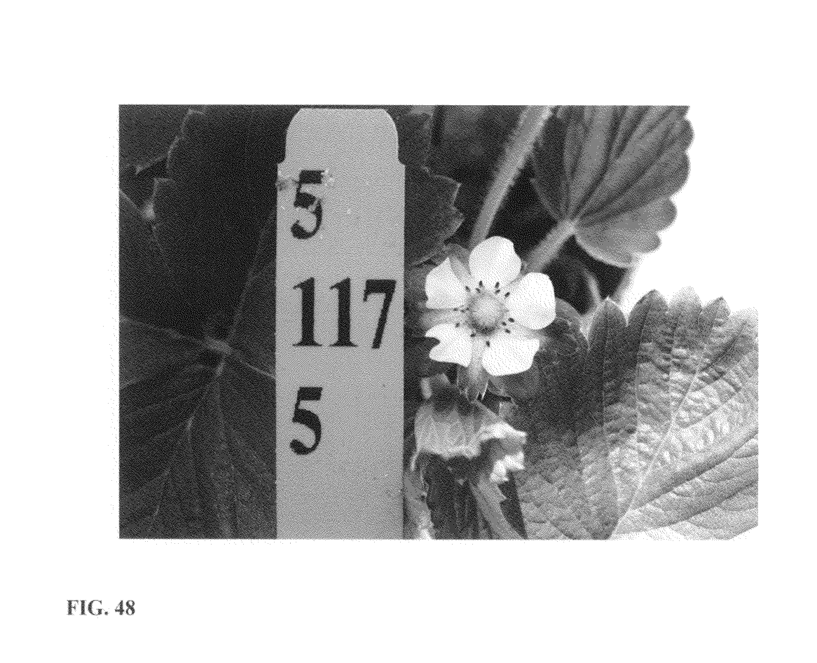

Cultivar ‘05.117-5’ has distinct disease/pathogen resistance, yield, appearance, size, and firmness. The plant of cultivar ‘05.117-5’ is characterized and distinguished in FIG. 46, the fruit of cultivar ‘05.117-5’ is characterized and distinguished in FIG. 47, and the flower of cultivar ‘05.117-5’ is characterized and distinguished in FIG. 48.

The following detailed description of cultivar ‘05.117-5’ is based on observations taken of plants and fruits (see FIGS. 46 and 47).

Certain characteristics of cultivar ‘05.117-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 16 provides data that characterizes the disease/pathogen resistance, yield, appearance, size, and firmness of cultivar ‘05.117-5’.

TABLE 16

| Cultivar | Vert | Phy | Fus | Yield | App | Size | Firm |
|---|---|---|---|---|---|---|---|
| 05.117-5 | 4.8 | 3.6 | 4.9 | 1442 | 3.7 | 35.5 | 13.3 |

### Cultivar ‘05.120-9’

In certain aspects, the present invention relates to a new and distinctive short-day type strawberry cultivar designated as ‘05.120-9’. Cultivar ‘05.120-9’ is typical of short-day strawberry cultivars and produces fruit over an extended period when treated appropriately in arid, subtropical climates.

The strawberry cultivar ‘05.120-9’ originated from a cross between cultivar ‘San Andreas’ (U.S. Plant Pat. No. 19,975) and unreleased germplasm accession ‘00.139-3’. The cultivar has been asexually reproduced by runners. The properties of this cultivar were found to be transmissible by such asexual reproduction. The cultivar is stable and reproduces true to type in successive generations of asexual reproduction.

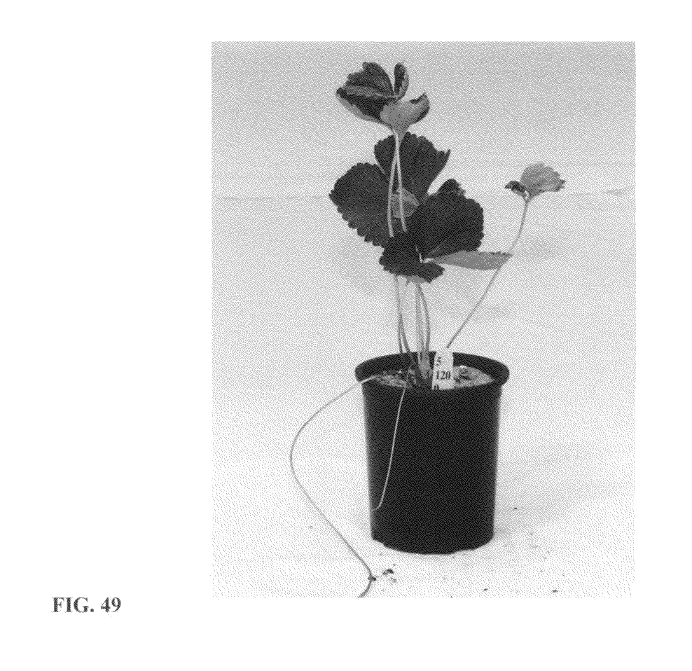

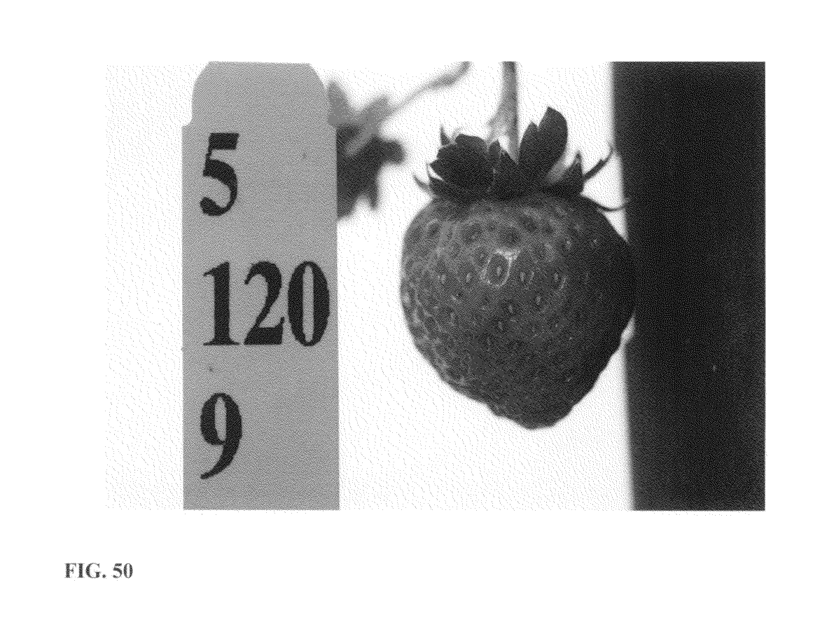

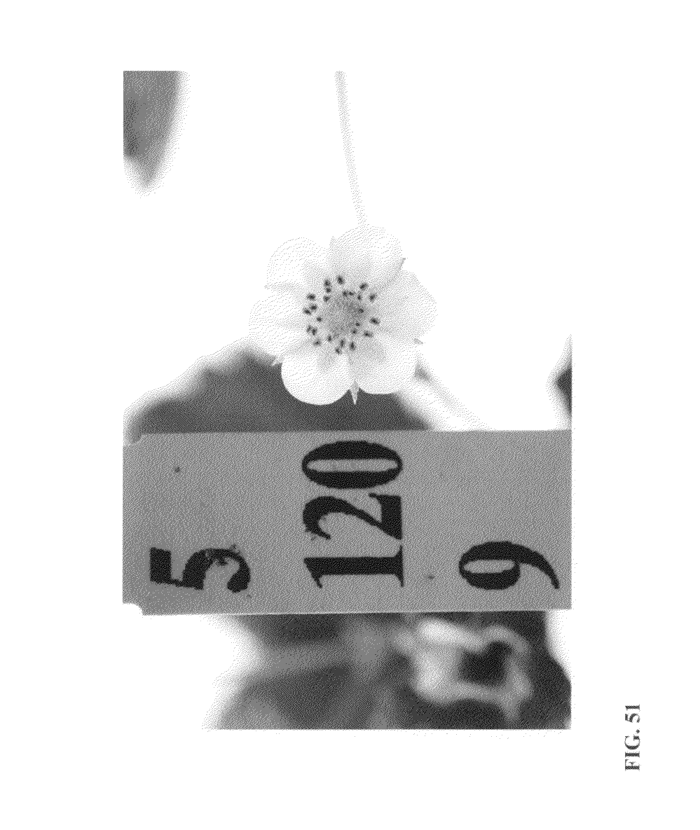

Cultivar ‘05.120-9’ has distinct disease/pathogen resistance, yield, appearance, size, and firmness. The plant of cultivar ‘05.120-9’ is characterized and distinguished in FIG. 49, the fruit of cultivar ‘05.120-9’ is characterized and distinguished in FIG. 50, and the flower of cultivar ‘05.120-9’ is characterized and distinguished in FIG. 51.

The following detailed description of cultivar ‘05.120-9’ is based on observations taken of plants and fruits (see FIGS. 49 and 50).

Certain characteristics of cultivar ‘05.120-9’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 17 provides data that characterizes the disease/pathogen resistance, yield, appearance, size, and firmness of cultivar ‘05.120-9’.

TABLE 17

| Cultivar | Vert | Phy | Yield | App | Size | Firm |
|---|---|---|---|---|---|---|
| 05.120-9 | 3.9 | 2.8 | 2657 | 4.1 | 41.4 | 11.3 |

### Cultivar ‘05.132-1’

In certain aspects, the present invention relates to a new and distinctive day-neutral type strawberry cultivar designated as ‘05.132-1’. Cultivar ‘05.132-1’ is typical of day-neutral strawberry cultivars and produces fruit regardless of day length when treated appropriately in arid, subtropical climates.

The strawberry cultivar ‘05.132-1’ originated from a cross between cultivar ‘Monterey’ (U.S. Plant Pat. No. 19,767) and unreleased germplasm accession ‘01.134-1’. The cultivar has been asexually reproduced by runners. The properties of this cultivar were found to be transmissible by such asexual reproduction. The cultivar is stable and reproduces true to type in successive generations of asexual reproduction.

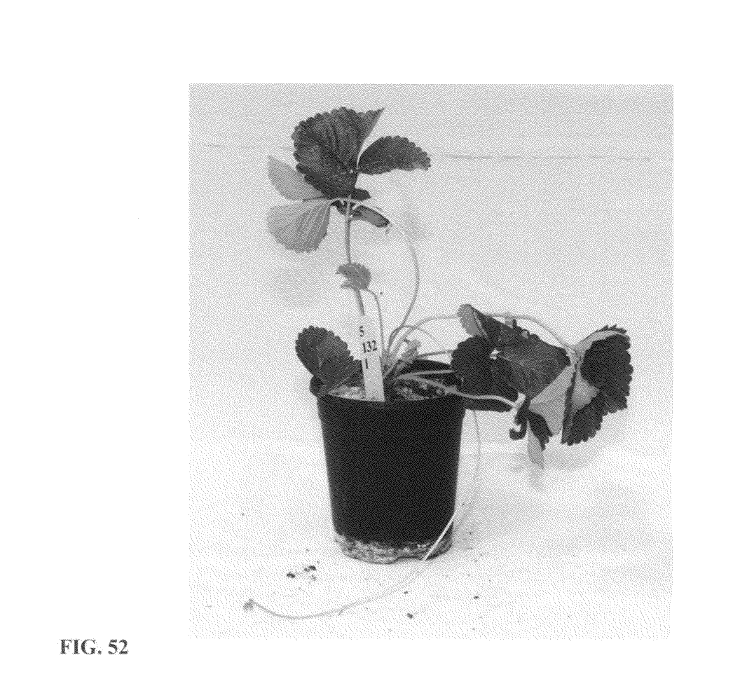

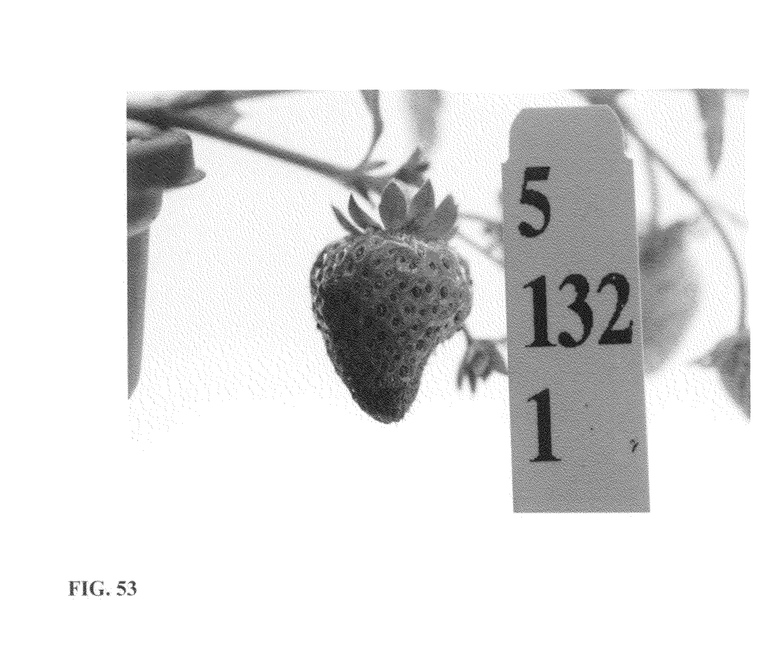

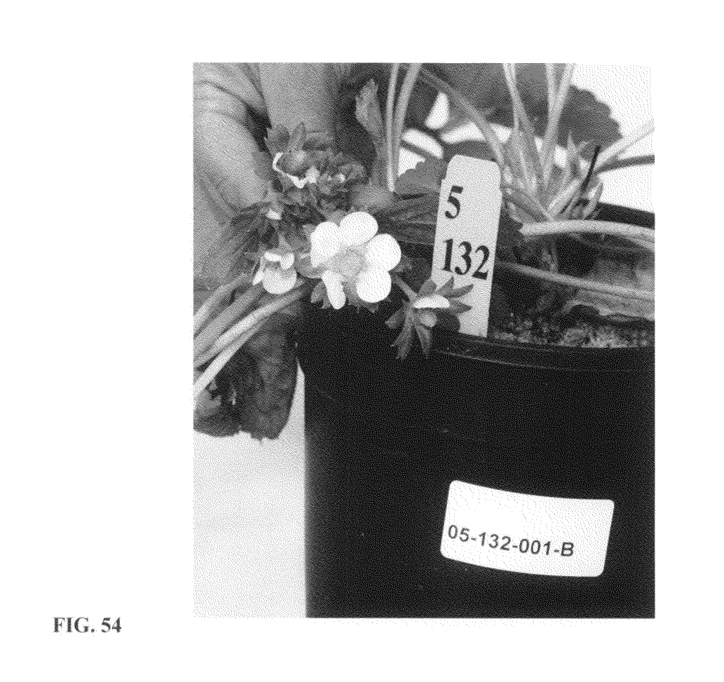

Cultivar ‘05.132-1’ has distinct disease/pathogen resistance, yield, appearance, size, and firmness. The plant of cultivar ‘05.132-1’ is characterized and distinguished in FIG. 52, the fruit of cultivar ‘05.132-1’ is characterized and distinguished in FIG. 53, and the flower of cultivar ‘05.132-1’ is characterized and distinguished in FIG. 54.

The following detailed description of cultivar ‘05.132-1’ is based on observations taken of plants and fruits (see FIGS. 52 and 53).

Certain characteristics of cultivar ‘05.132-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 18 provides data that characterizes the disease/pathogen resistance, yield, appearance, size, and firmness of cultivar ‘05.132-1’.

TABLE 18

| Cultivar | Vert | Phy | Yield | App | Size | Firm |
|---|---|---|---|---|---|---|
| 05.132-1 | 2.6 | 3.8 | 2612 | 3.5 | 38.4 | 11.9 |

### Cultivar ‘05.148-2’

In certain aspects, the present invention relates to a new and distinctive short-day type strawberry cultivar designated as ‘05.148-2’. Cultivar ‘05.148-2’ is typical of short-day strawberry cultivars and produces fruit over an extended period when treated appropriately in arid, subtropical climates.

The strawberry cultivar ‘05.148-2’ originated from a cross between cultivar ‘San Andreas’ (U.S. Plant Pat. No. 19,975) and unreleased germplasm accession ‘02.229-1’. The cultivar has been asexually reproduced by runners. The properties of this cultivar were found to be transmissible by such asexual reproduction. The cultivar is stable and reproduces true to type in successive generations of asexual reproduction.

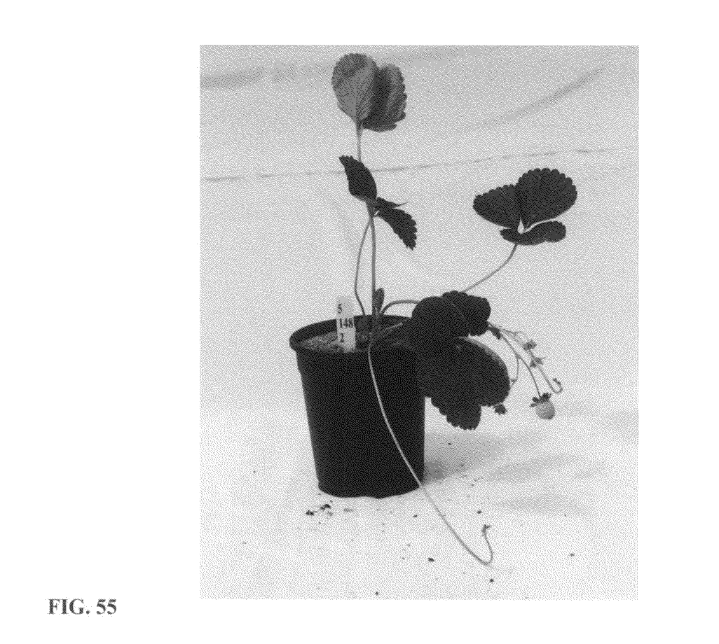

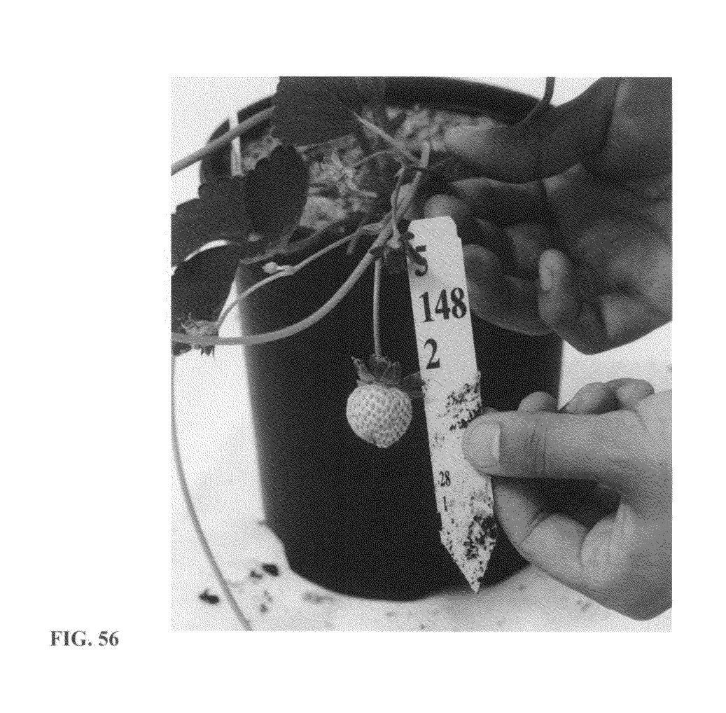

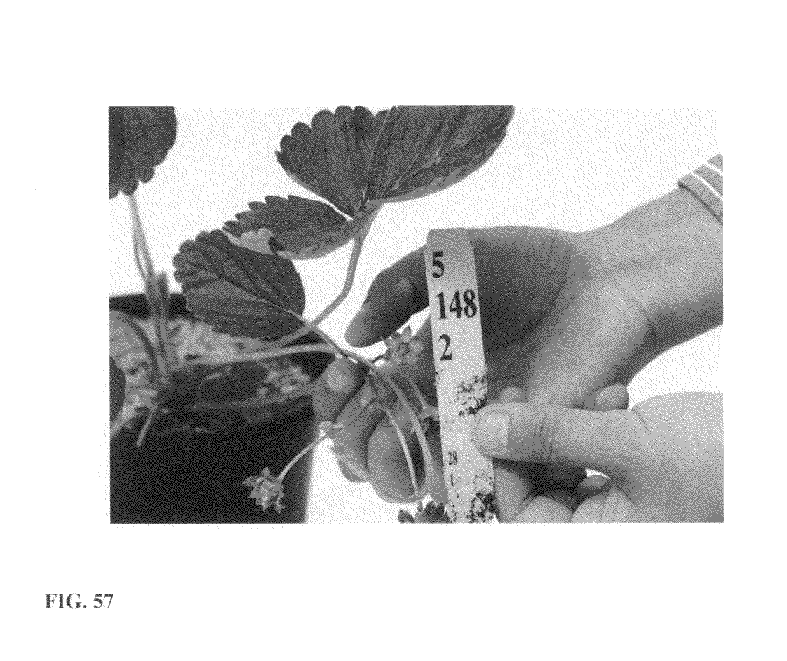

Cultivar ‘05.148-2’ has distinct disease/pathogen resistance, yield, appearance, size, and firmness. The plant of cultivar ‘05.148-2’ is characterized and distinguished in FIG. 55, the fruit of cultivar ‘05.148-2’ is characterized and distinguished in FIG. 56, and the flower of cultivar ‘05.148-2’ is characterized and distinguished in FIG. 57.

The following detailed description of cultivar ‘05.148-2’ is based on observations taken of plants and fruits (see FIGS. 55 and 56).

Certain characteristics of cultivar ‘05.148-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 19 provides data that characterizes the disease/pathogen resistance, yield, appearance, size, and firmness of cultivar ‘05.148-2’.

TABLE 19

| Cultivar | Vert | Phy | Fus | Yield | App | Size | Firm |
|---|---|---|---|---|---|---|---|
| 05.148-2 | 4.7 | 4.5 | 5.0 | 2082 | 3.5 | 34.1 | 12.9 |

### Cultivar ‘05.165-1’

In certain aspects, the present invention relates to a new and distinctive short-day type strawberry cultivar designated as ‘05.165-1’. Cultivar ‘05.165-1’ is typical of short-day strawberry cultivars and produces fruit over an extended period when treated appropriately in arid, subtropical climates.

The strawberry cultivar ‘05.165-1’ originated from a cross between two unreleased germplasm accessions ‘02.143-3’ and ‘01.111-4’. The cultivar has been asexually reproduced by runners. The properties of this cultivar were found to be transmissible by such asexual reproduction. The cultivar is stable and reproduces true to type in successive generations of asexual reproduction.

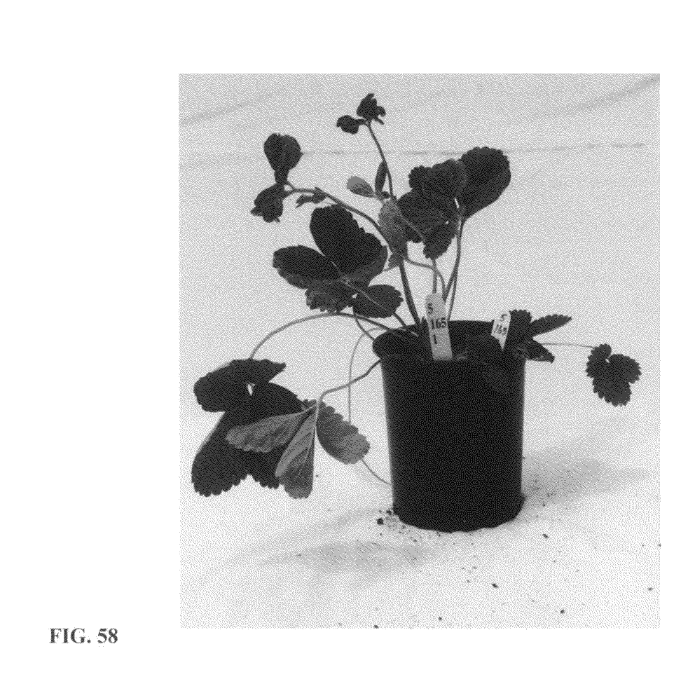

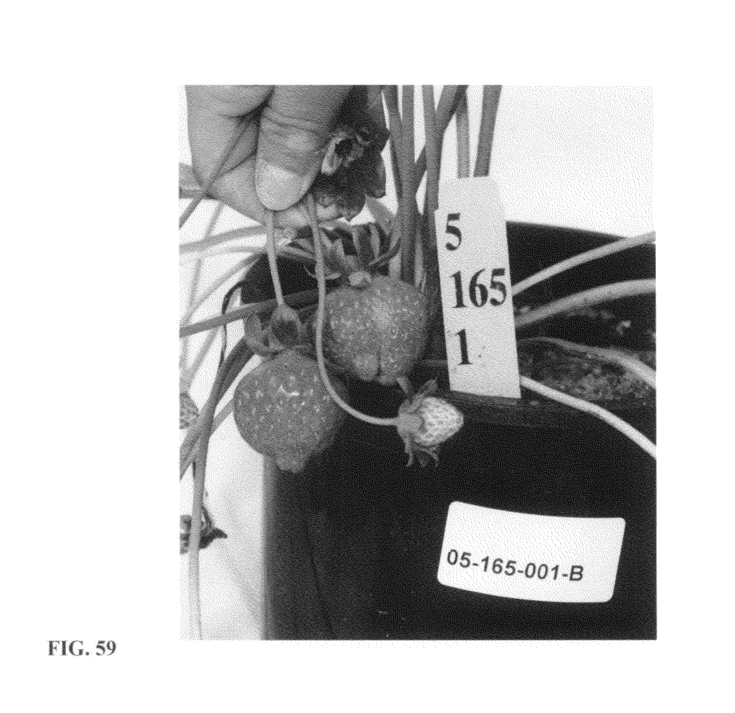

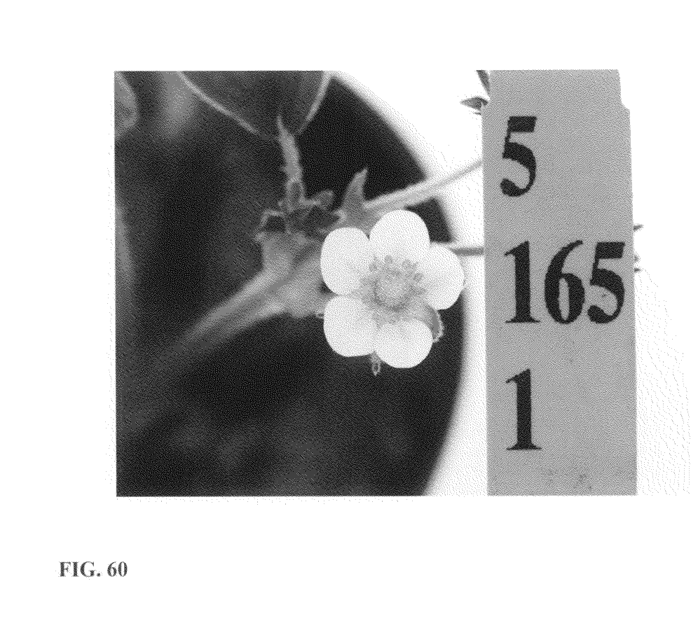

Cultivar ‘05.165-1’ has distinct disease/pathogen resistance, yield, appearance, size, and firmness. The plant of cultivar ‘05.165-1’ is characterized and distinguished in FIG. 58, the fruit of cultivar ‘05.165-1’ is characterized and distinguished in FIG. 59, and the flower of cultivar ‘05.165-1’ is characterized and distinguished in FIG. 60.

The following detailed description of cultivar ‘05.165-1’ is based on observations taken of plants and fruits (see FIGS. 58 and 59).

Certain characteristics of cultivar ‘05.165-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 20 provides data that characterizes the disease/pathogen resistance, yield, appearance, size, and firmness of cultivar ‘05.165-1’.

TABLE 20

| Cultivar | Vert | Phy | Fus | Yield | App | Size | Firm |
|---|---|---|---|---|---|---|---|
| 05.165-1 | 3.9 | 4.0 | 1.0 | 1620 | 3.6 | 34.8 | 12.4 |

### Cultivar ‘05.197-2’

In certain aspects, the present invention relates to a new and distinctive day-neutral type strawberry cultivar designated as ‘05.197-2’. Cultivar ‘05.197-2’ is typical of day-neutral strawberry cultivars and produces fruit regardless of day length when treated appropriately in arid, subtropical climates.

The strawberry cultivar ‘05.197-2’ originated from a cross between cultivar ‘San Andreas’ (U.S. Plant Pat. No. 19,975) and unreleased germplasm accession ‘01.134-3’. The cultivar has been asexually reproduced by runners. The properties of this cultivar were found to be transmissible by such asexual reproduction. The cultivar is stable and reproduces true to type in successive generations of asexual reproduction.

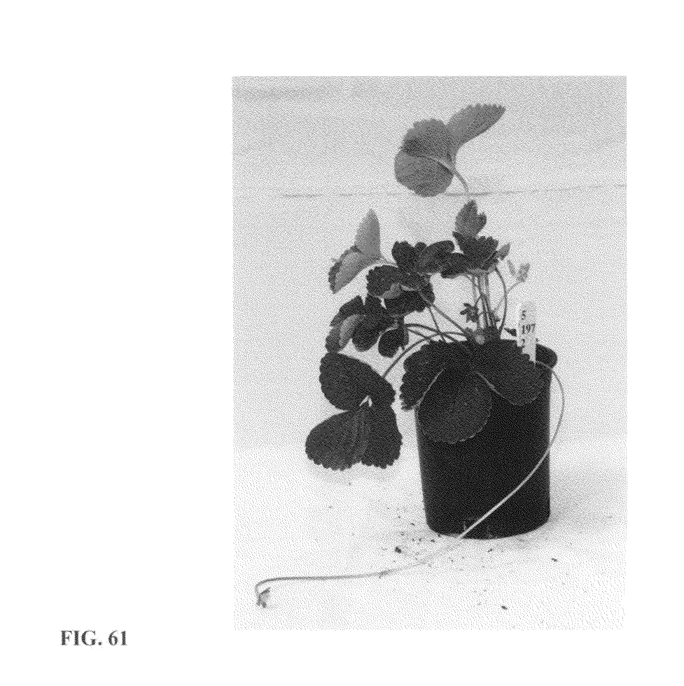

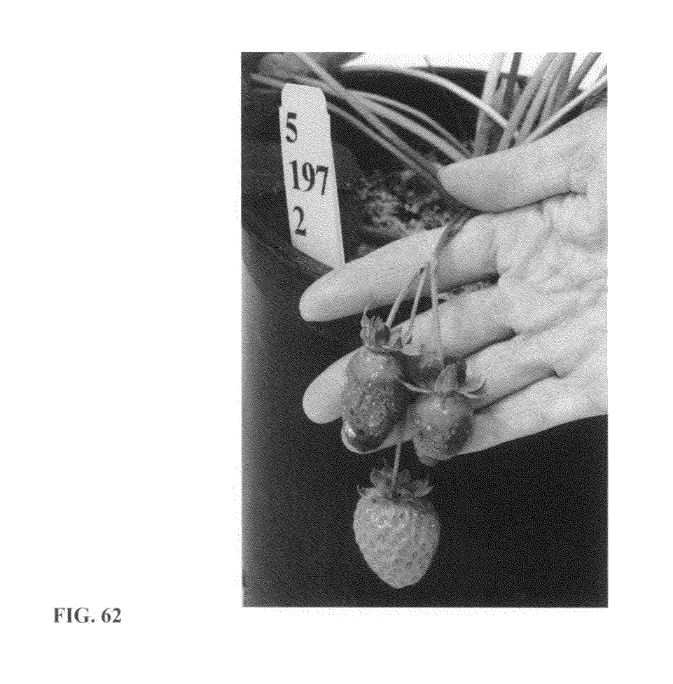

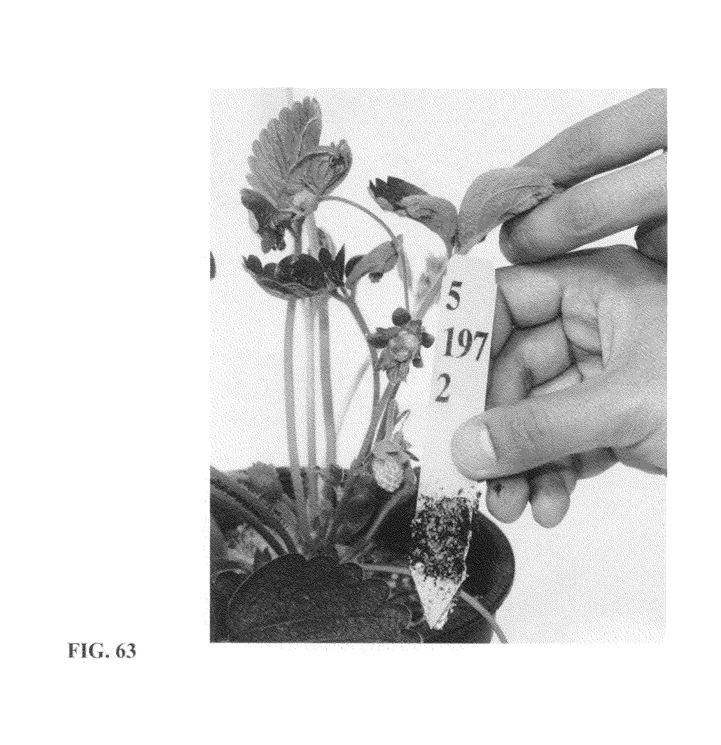

Cultivar ‘05.197-2’ has distinct disease/pathogen resistance, yield, appearance, size, and firmness. The plant of cultivar ‘05.197-2’ is characterized and distinguished in FIG. 61, the fruit of cultivar ‘05.197-2’ is characterized and distinguished in FIG. 62, and the flower of cultivar ‘05.197-2’ is characterized and distinguished in FIG. 63.

The following detailed description of cultivar ‘05.197-2’ is based on observations taken of plants and fruits (see FIGS. 61 and 62).

Certain characteristics of cultivar ‘05.197-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 21 provides data that characterizes the disease/pathogen resistance, yield, appearance, size, and firmness of cultivar ‘05.197-2’.

TABLE 21

| Cultivar | Vert | Phy | Yield | App | Size | Firm |
|---|---|---|---|---|---|---|
| 05.197-2 | 5.0 | 4.1 | 2707 | 3.7 | 40.0 | 12.1 |

### Cultivar ‘05.205-2’

In certain aspects, the present invention relates to a new and distinctive day-neutral type strawberry cultivar designated as ‘05.205-2’. Cultivar ‘05.205-2’ is typical of day-neutral strawberry cultivars and produces fruit regardless of day length when treated appropriately in arid, subtropical climates.

The strawberry cultivar ‘05.205-2’ originated from a cross between cultivar ‘San Andreas’ (U.S. Plant Pat. No. 19,975) and unreleased germplasm accession ‘02.134-3’. The cultivar has been asexually reproduced by runners. The properties of this cultivar were found to be transmissible by such asexual reproduction. The cultivar is stable and reproduces true to type in successive generations of asexual reproduction.

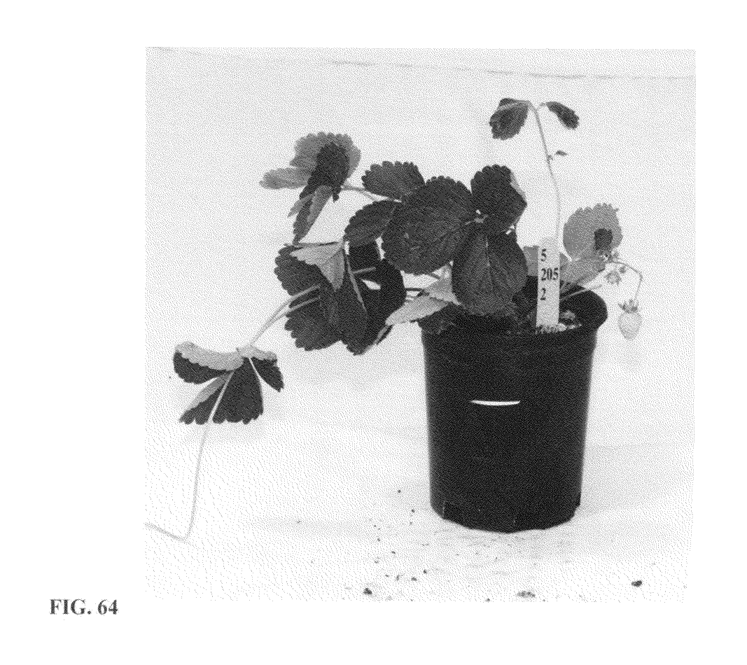

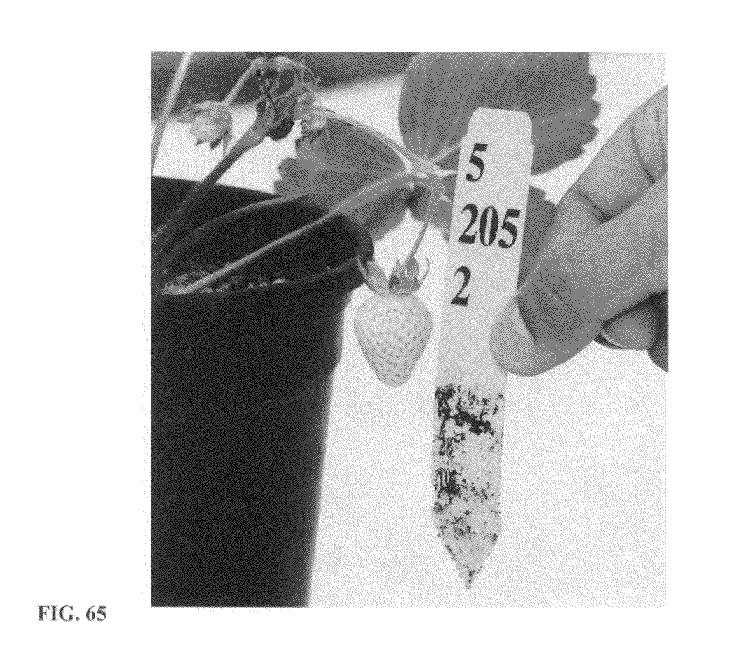

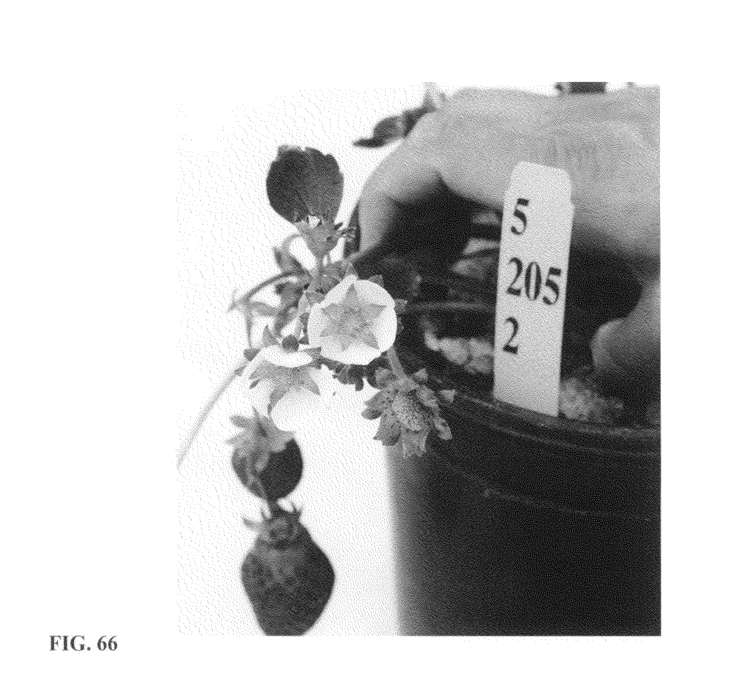

Cultivar ‘05.205-2’ has distinct disease/pathogen resistance, yield, appearance, size, and firmness. The plant of cultivar ‘05.205-2’ is characterized and distinguished in FIG. 64, the fruit of cultivar ‘05.205-2’ is characterized and distinguished in FIG. 65, and the flower of cultivar ‘05.205-2’ is characterized and distinguished in FIG. 66.

The following detailed description of cultivar ‘05.205-2’ is based on observations taken of plants and fruits (see FIGS. 64 and 65).

Certain characteristics of cultivar ‘05.205-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 22 provides data that characterizes the disease/pathogen resistance, yield, appearance, size, and firmness of cultivar ‘05.205-2’.

TABLE 22

| Cultivar | Vert | Phy | Yield | App | Size | Firm |
|---|---|---|---|---|---|---|
| 05.205-2 | 3.9 | 2.8 | 2673 | 3.9 | 39.1 | 9.4 |

### Cultivar ‘05.206-5’

In certain aspects, the present invention relates to a new and distinctive day-neutral type strawberry cultivar designated as ‘05.206-5’. Cultivar ‘05.206-5’ is typical of day-neutral strawberry cultivars and produces fruit regardless of day length when treated appropriately in arid, subtropical climates.

The strawberry cultivar ‘05.206-5’ originated from a cross between two unreleased germplasm accessions ‘02.143-3’ and ‘02.134-3’. The cultivar has been asexually reproduced by runners. The properties of this cultivar were found to be transmissible by such asexual reproduction. The cultivar is stable and reproduces true to type in successive generations of asexual reproduction.

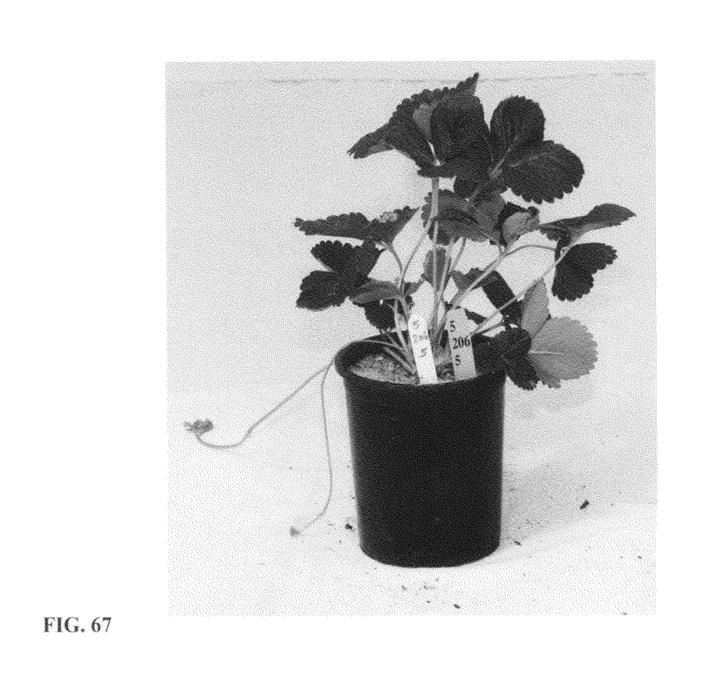

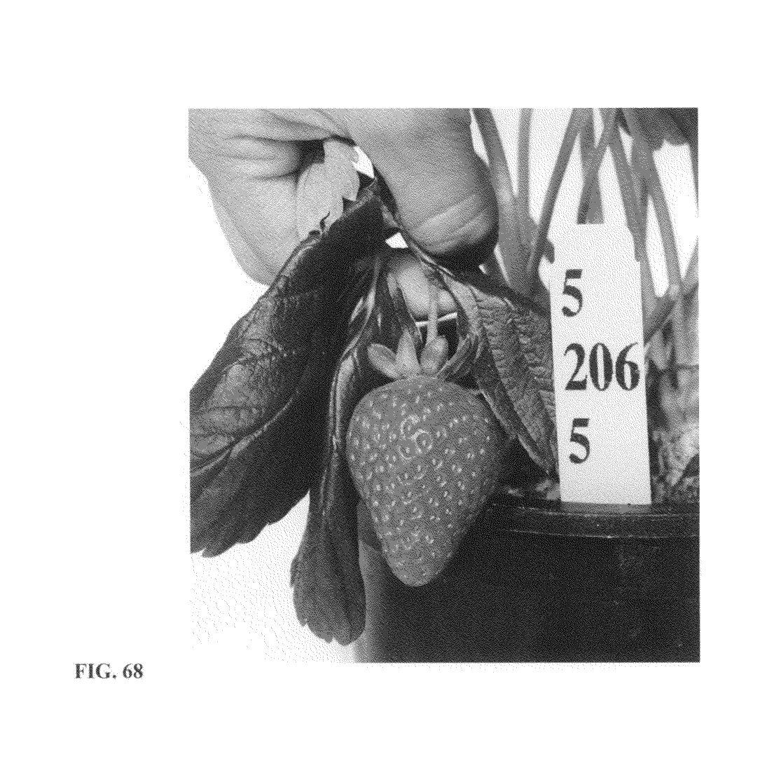

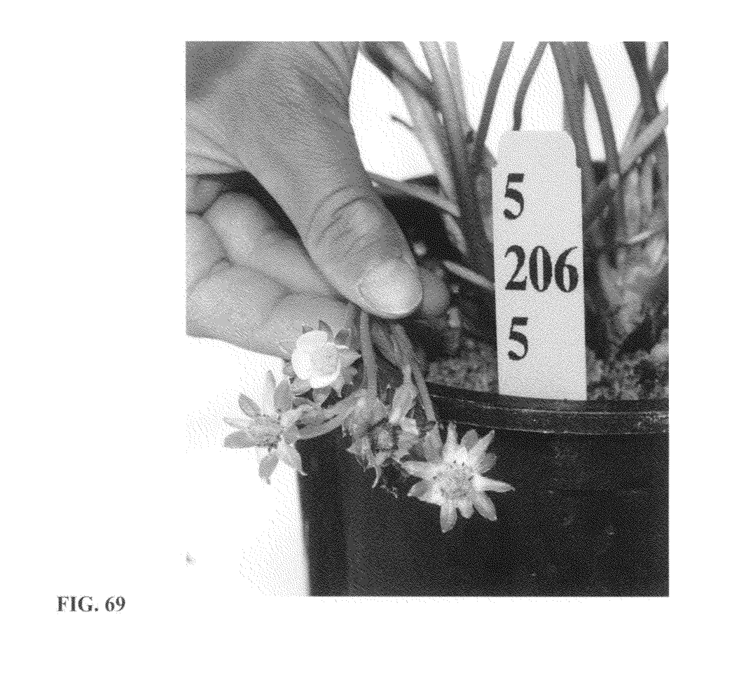

Cultivar ‘05.206-5’ has distinct disease/pathogen resistance, yield, appearance, size, and firmness. The plant of cultivar ‘05.206-5’ is characterized and distinguished in FIG. 67, the fruit of cultivar ‘05.206-5’ is characterized and distinguished in FIG. 68, and the flower of cultivar ‘05.206-5’ is characterized and distinguished in FIG. 69.

The following detailed description of cultivar ‘05.206-5’ is based on observations taken of plants and fruits (see FIGS. 67 and 68).

Certain characteristics of cultivar ‘05.206-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 23 provides data that characterizes the disease/pathogen resistance, yield, appearance, size, and firmness of cultivar ‘05.206-5’.

TABLE 23

| Cultivar | Vert | Phy | Fus | Yield | App | Size | Firm |
|---|---|---|---|---|---|---|---|
| 05.206-5 | 5.0 | 3.9 | 1.9 | 2341 | 3.9 | 33.3 | 14.7 |

### Cultivar ‘06.16-602’

In certain aspects, the present invention relates to a new and distinctive short-day type strawberry cultivar designated as ‘06.16-602’. Cultivar ‘06.16-602’ is typical of short-day strawberry cultivars and produces fruit over an extended period when treated appropriately in arid, subtropical climates.

The strawberry cultivar ‘06.16-602’ originated from a cross between two unreleased germplasm accessions ‘01.80-602’ and ‘03.75-601’. The cultivar has been asexually reproduced by runners. The properties of this cultivar were found to be transmissible by such asexual reproduction. The cultivar is stable and reproduces true to type in successive generations of asexual reproduction.

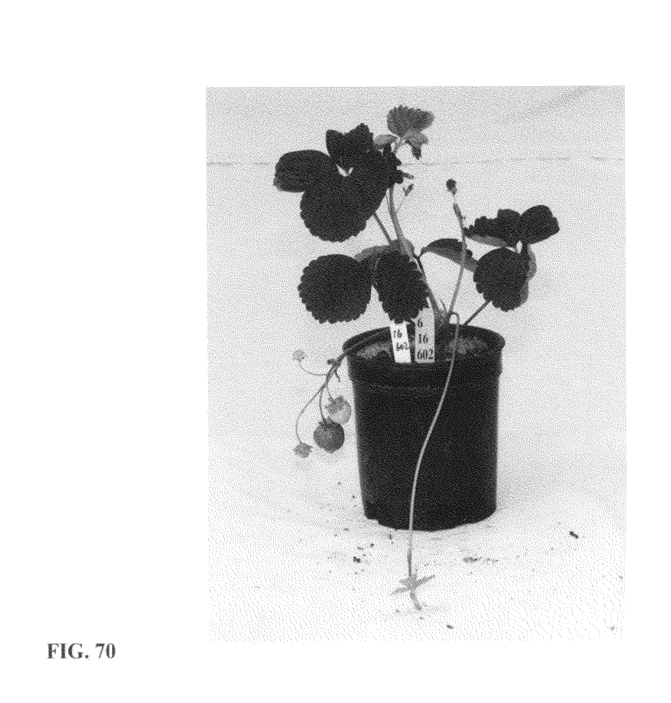

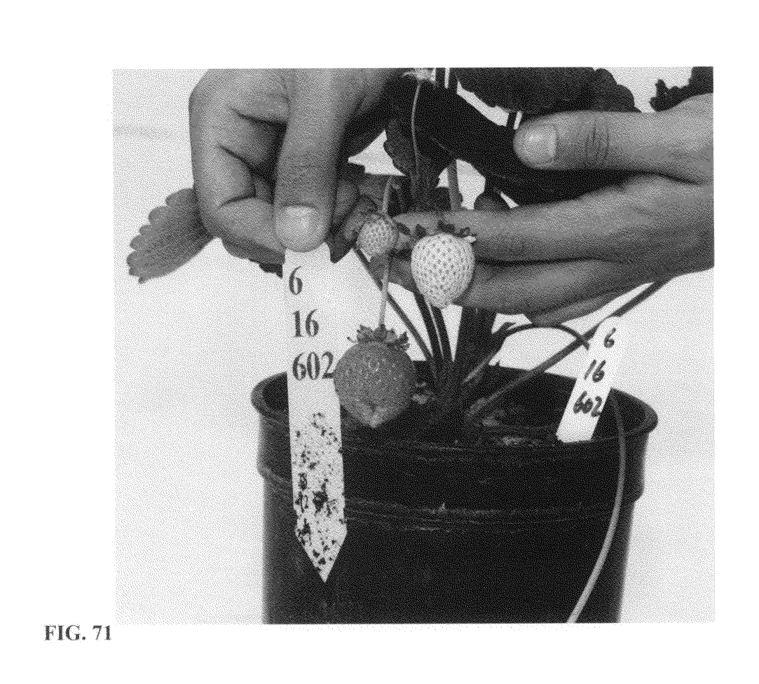

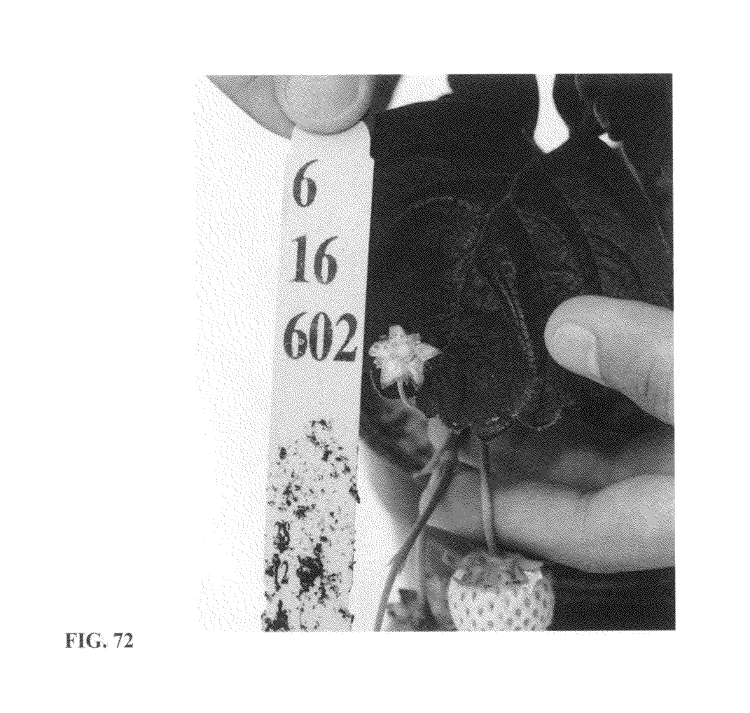

Cultivar ‘06.16-602’ has distinct disease/pathogen resistance, yield, appearance, size, and firmness. The plant of cultivar ‘06.16-602’ is characterized and distinguished in FIG. 70, the fruit of cultivar ‘06.16-602’ is characterized and distinguished in FIG. 71, and the flower of cultivar ‘06.16-602’ is characterized and distinguished in FIG. 72.

The following detailed description of cultivar ‘06.16-602’ is based on observations taken of plants and fruits (see FIGS. 70 and 71).

Certain characteristics of cultivar ‘06.16-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 24 provides data that characterizes the disease/pathogen resistance, yield, appearance, size, and firmness of cultivar ‘06.16-602’.

TABLE 24

| Cultivar | Vert | Phy | Yield | App | Size | Firm |
|---|---|---|---|---|---|---|
| 06.16-602 | 4.9 | 4.1 | 1777 | 3.7 | 40.9 | 10.8 |

### Cultivar ‘06.20-605’

In certain aspects, the present invention relates to a new and distinctive short-day type strawberry cultivar designated as ‘06.20-605’. Cultivar ‘06.20-605’ is typical of short-day strawberry cultivars and produces fruit over an extended period when treated appropriately in arid, subtropical climates.

The strawberry cultivar ‘06.20-605’ originated from a cross between two unreleased germplasm accessions ‘03.65-601’ and ‘03.75-601’. The cultivar has been asexually reproduced by runners. The properties of this cultivar were found to be transmissible by such asexual reproduction. The cultivar is stable and reproduces true to type in successive generations of asexual reproduction.

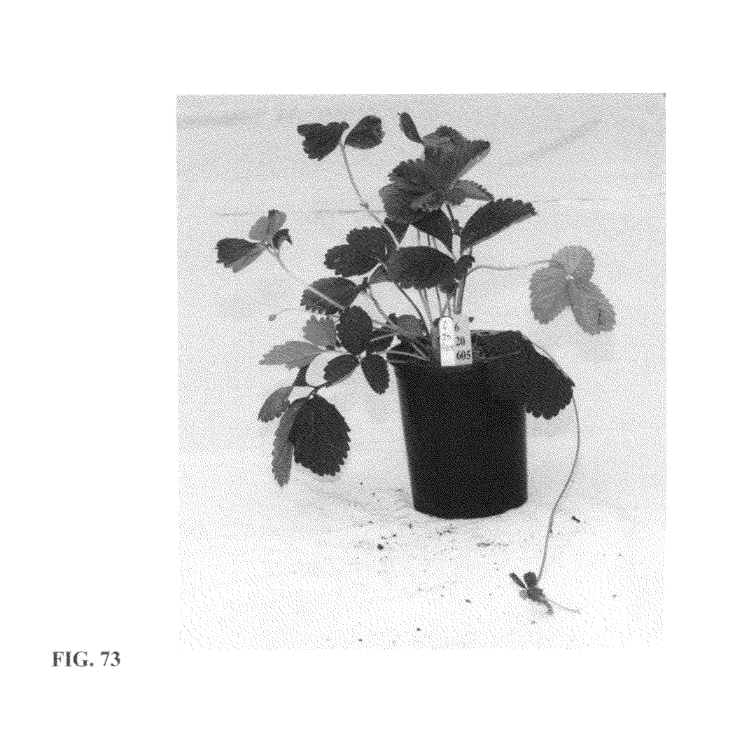

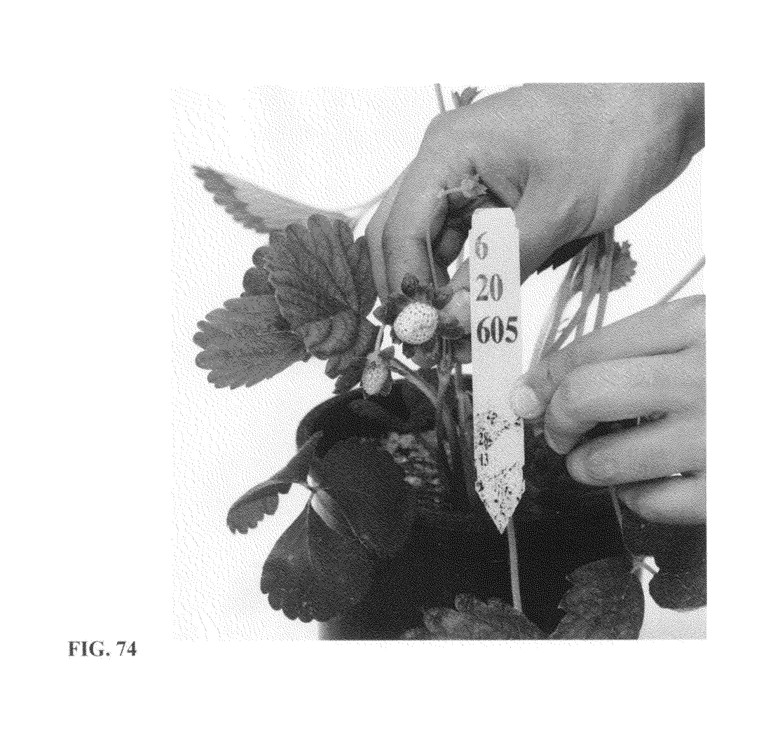

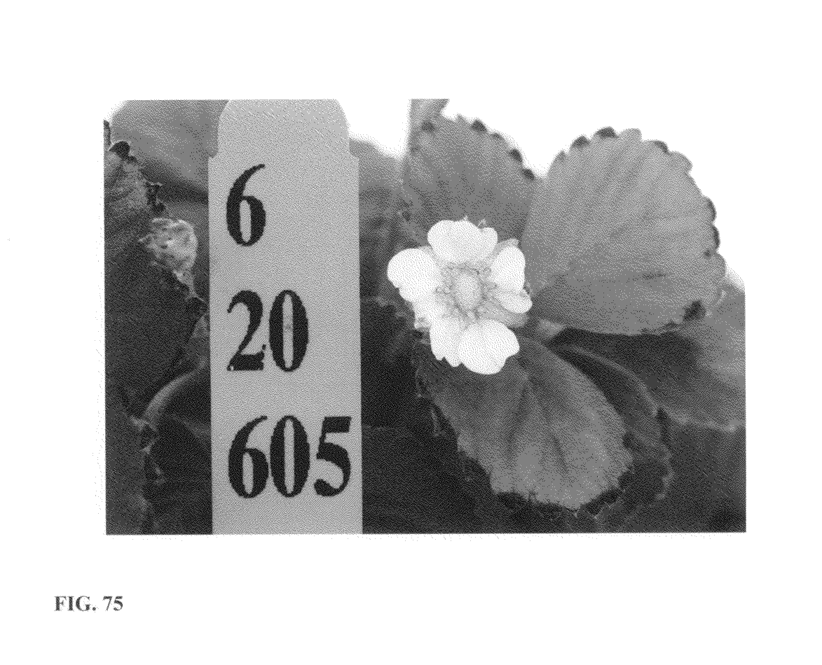

Cultivar ‘06.20-605’ has distinct disease/pathogen resistance, yield, appearance, size, and firmness. The plant of cultivar ‘06.20-605’ is characterized and distinguished in FIG. 73, the fruit of cultivar ‘06.20-605’ is characterized and distinguished in FIG. 74, and the flower of cultivar ‘06.20-605’ is characterized and distinguished in FIG. 75.

The following detailed description of cultivar ‘06.20-605’ is based on observations taken of plants and fruits (see FIGS. 73 and 74).

Certain characteristics of cultivar ‘06.20-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 25 provides data that characterizes the disease/pathogen resistance, yield, appearance, size, and firmness of cultivar ‘06.20-605’.

TABLE 25

| Cultivar | Vert | Fus | Yield | App | Size | Firm |
|---|---|---|---|---|---|---|
| 06.20-605 | 4.9 | 4.9 | 2137 | 3.4 | 37.7 | 11.7 |

### Cultivar ‘06.51-609’

In certain aspects, the present invention relates to a new and distinctive short-day type strawberry cultivar designated as ‘06.51-609’. Cultivar ‘06.51-609’ is typical of short-day strawberry cultivars and produces fruit over an extended period when treated appropriately in arid, subtropical climates.

The strawberry cultivar ‘06.51-609’ originated from a cross between two unreleased germplasm accessions ‘02.144-3’ and ‘02.68-608’. The cultivar has been asexually reproduced by runners. The properties of this cultivar were found to be transmissible by such asexual reproduction. The cultivar is stable and reproduces true to type in successive generations of asexual reproduction.

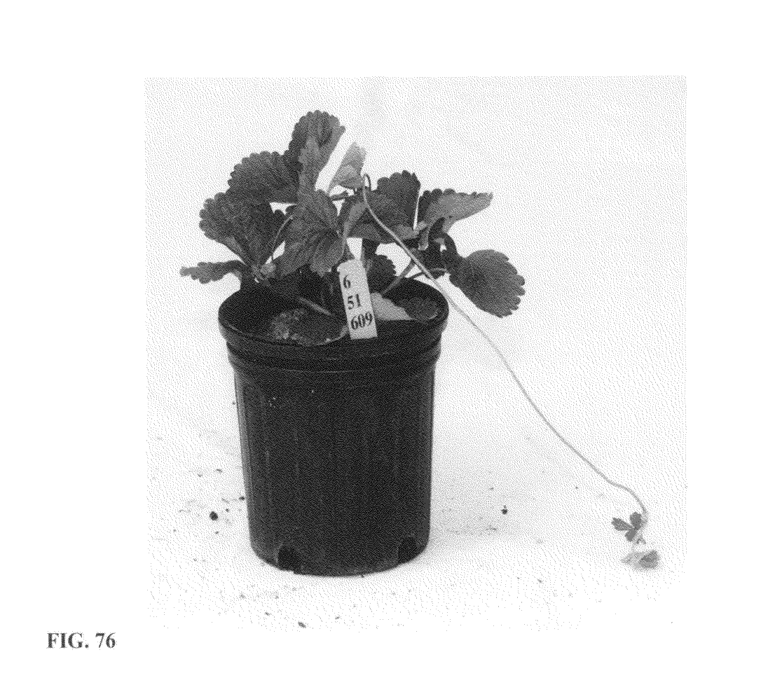

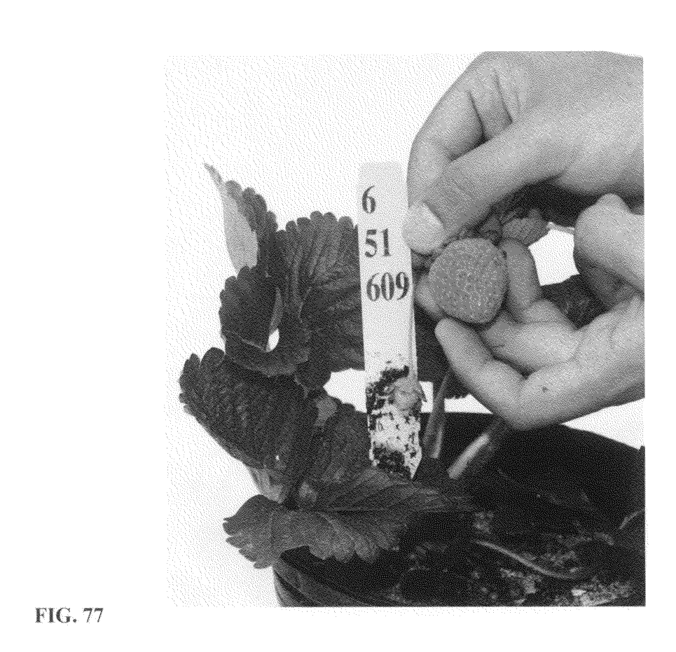

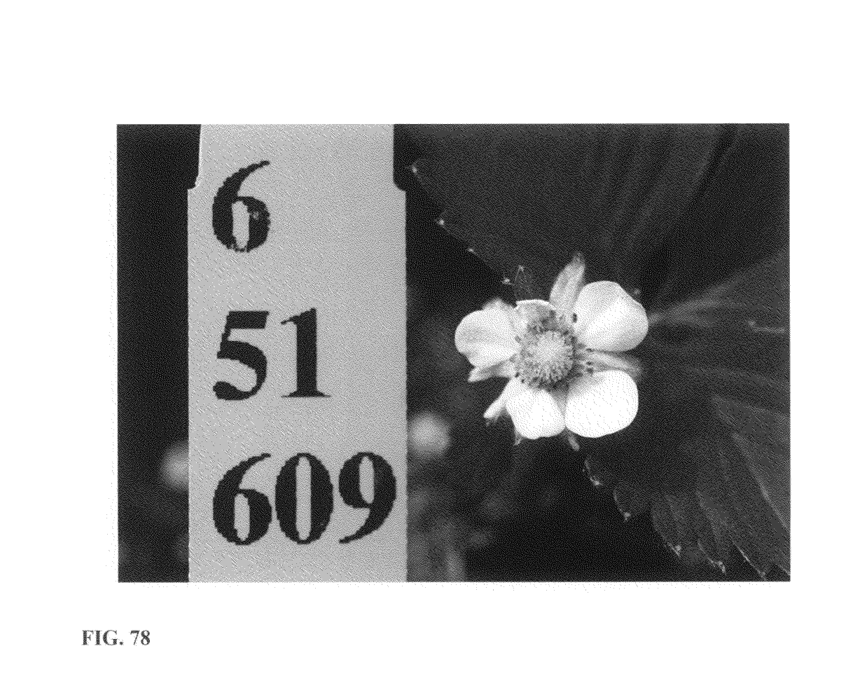

Cultivar ‘06.51-609’ has distinct disease/pathogen resistance, yield, appearance, size, and firmness. The plant of cultivar ‘06.51-609’ is characterized and distinguished in FIG. 76, the fruit of cultivar ‘06.51-609’ is characterized and distinguished in FIG. 77, and the flower of cultivar ‘06.51-609’ is characterized and distinguished in FIG. 78.

The following detailed description of cultivar ‘06.51-609’ is based on observations taken of plants and fruits (see FIGS. 76 and 77).

Certain characteristics of cultivar ‘06.51-609’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 26 provides data that characterizes the disease/pathogen resistance, yield, appearance, size, and firmness of cultivar ‘06.51-609’.

TABLE 26

| Cultivar | Vert | Phy | Fus | Yield | App | Size | Firm |
|---|---|---|---|---|---|---|---|
| 06.51-609 | 3.3 | 4.0 | 2.6 | 2034 | 3.9 | 32.4 | 11.8 |

### Cultivar ‘06.137-2’

In certain aspects, the present invention relates to a new and distinctive short-day type strawberry cultivar designated as ‘06.137-2’. Cultivar ‘06.137-2’ is typical of short-day strawberry cultivars and produces fruit over an extended period when treated appropriately in arid, subtropical climates.

The strawberry cultivar ‘06.137-2’ originated from a cross between two unreleased germplasm accessions ‘02.143-3’ and ‘02.132-6’. The cultivar has been asexually reproduced by runners. The properties of this cultivar were found to be transmissible by such asexual reproduction. The cultivar is stable and reproduces true to type in successive generations of asexual reproduction.

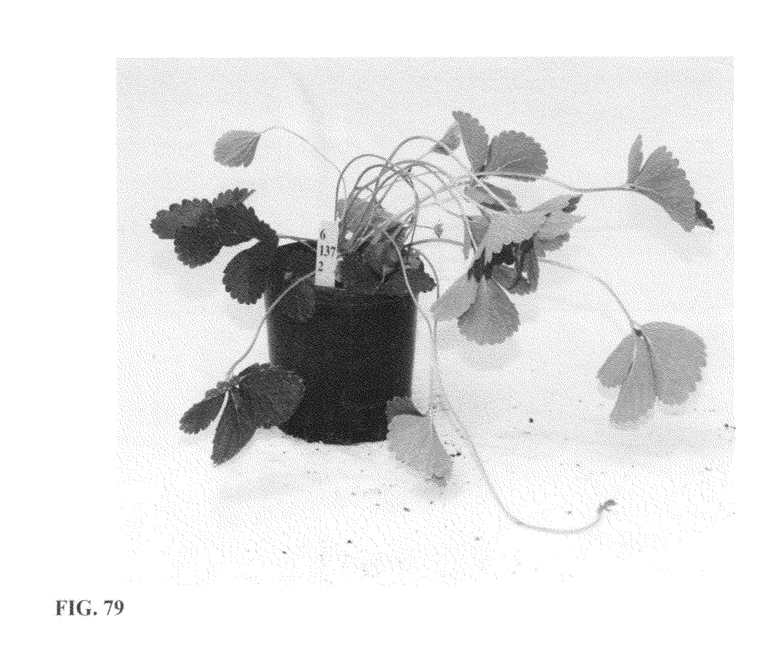

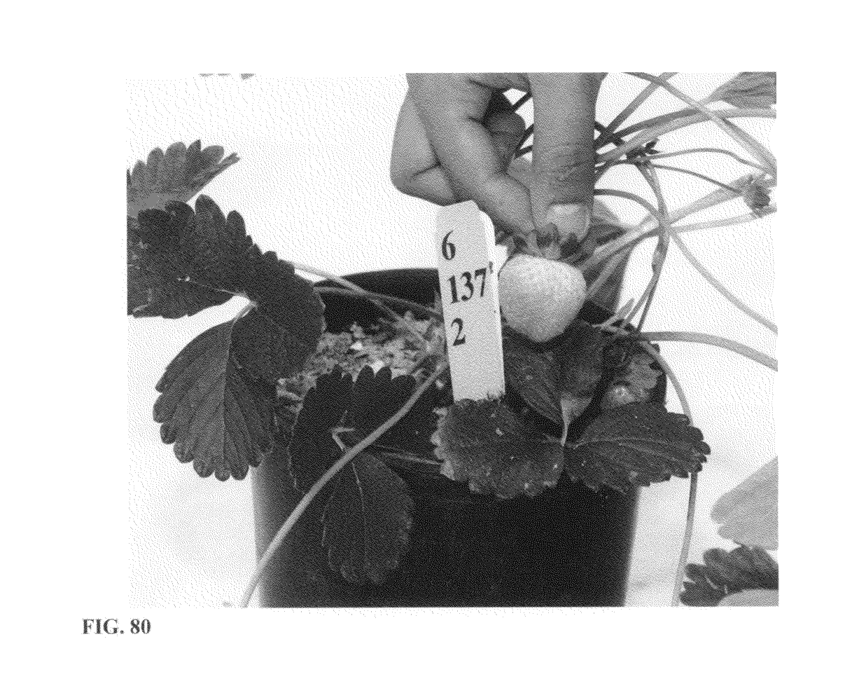

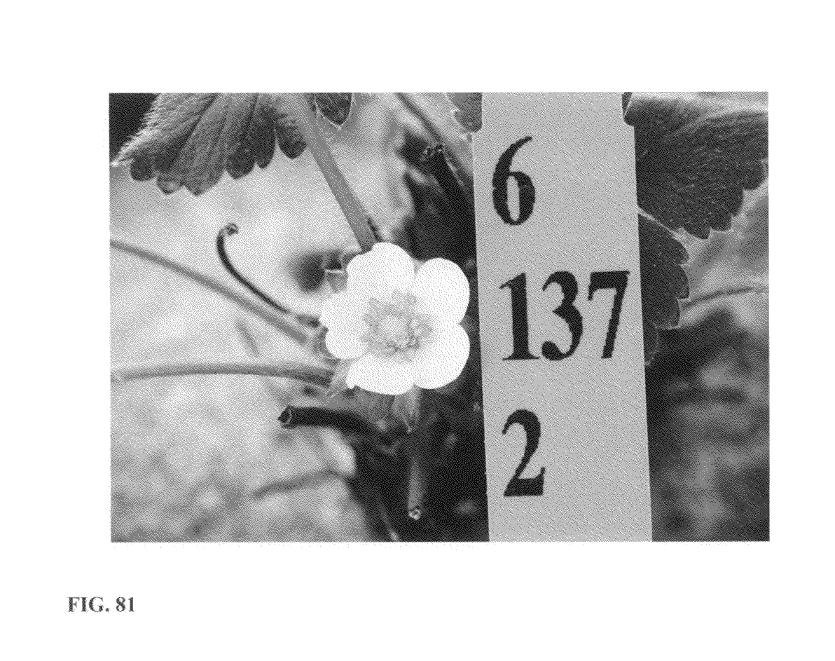

Cultivar ‘06.137-2’ has distinct disease/pathogen resistance, yield, appearance, size, and firmness. The plant of cultivar ‘06.137-2’ is characterized and distinguished in FIG. 79, the fruit of cultivar ‘06.137-2’ is characterized and distinguished in FIG. 80, and the flower of cultivar ‘06.137-2’ is characterized and distinguished in FIG. 81.

The following detailed description of cultivar ‘06.137-2’ is based on observations taken of plants and fruits (see FIGS. 79 and 80).

Certain characteristics of cultivar ‘06.137-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 27 provides data that characterizes the disease/pathogen resistance, yield, appearance, size, and firmness of cultivar ‘06.137-2’.

TABLE 27

| Cultivar | Vert | Phy | Fus | Yield | App | Size | Firm |
|---|---|---|---|---|---|---|---|
| 06.137-2 | 4.8 | 4.1 | 2.5 | 1630 | 4.3 | 38.5 | 12.8 |

### Cultivar ‘06.204-1’

In certain aspects, the present invention relates to a new and distinctive day-neutral type strawberry cultivar designated as ‘06.204-1’. Cultivar ‘06.204-1’ is typical of day-neutral strawberry cultivars and produces fruit regardless of day length when treated appropriately in arid, subtropical climates.

The strawberry cultivar ‘06.204-1’ originated from a cross between unreleased germplasm accession ‘03.97-1’ and cultivar ‘San Andreas’ (U.S. Plant Pat. No. 19,975). The cultivar has been asexually reproduced by runners. The properties of this cultivar were found to be transmissible by such asexual reproduction. The cultivar is stable and reproduces true to type in successive generations of asexual reproduction.

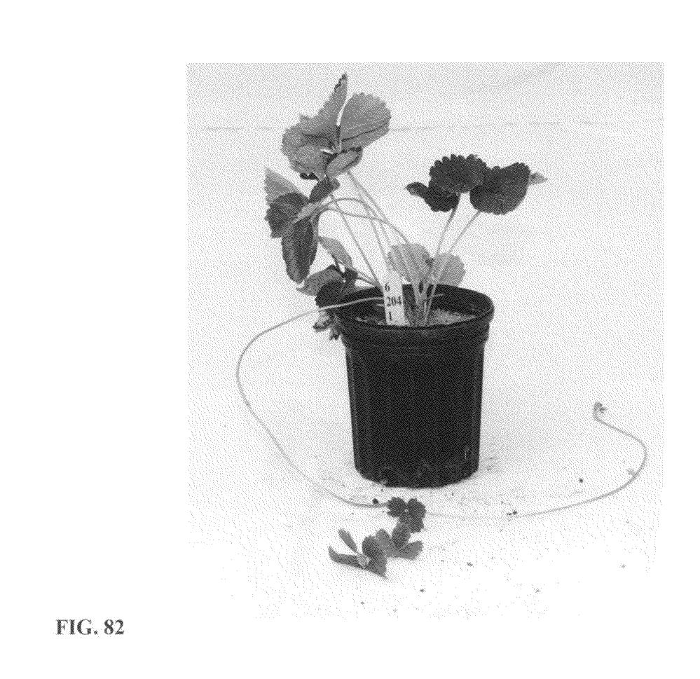

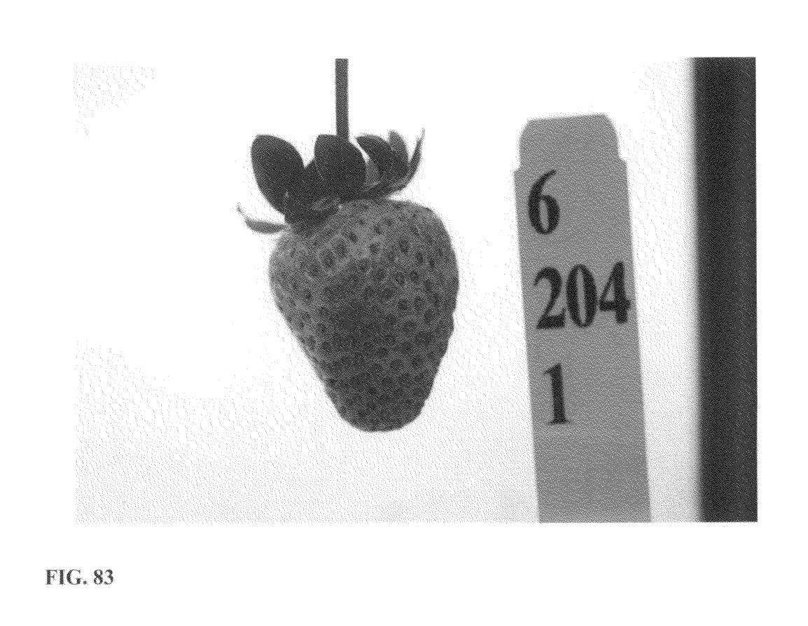

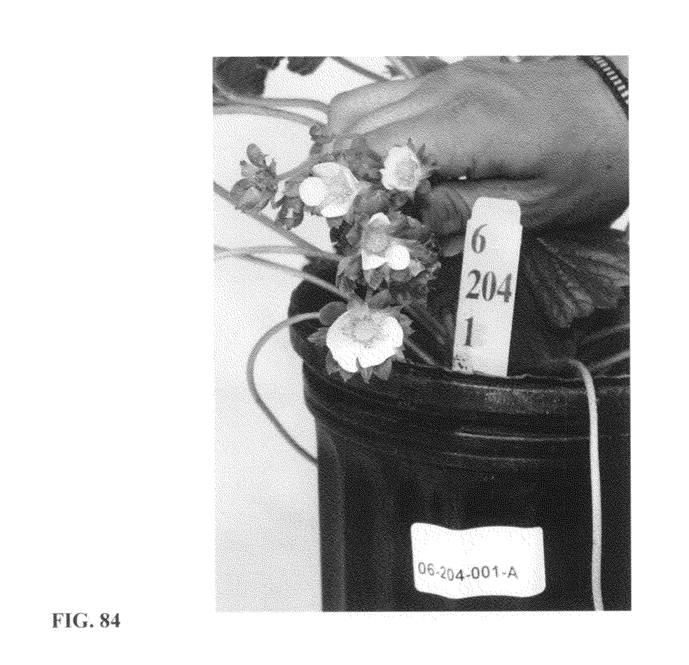

Cultivar ‘06.204-1’ has distinct disease/pathogen resistance, yield, appearance, size, and firmness. The plant of cultivar ‘06.204-1’ is characterized and distinguished in FIG. 82, the fruit of cultivar ‘06.204-1’ is characterized and distinguished in FIG. 83, and the flower of cultivar ‘06.204-1’ is characterized and distinguished in FIG. 84.

The following detailed description of cultivar ‘06.204-1’ is based on observations taken of plants and fruits (see FIGS. 82 and 83).

Certain characteristics of cultivar ‘06.204-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 28 provides data that characterizes the disease/pathogen resistance, yield, appearance, size, and firmness of cultivar ‘06.204-1’.

TABLE 28

| Cultivar | Vert | Phy | Fus | Yield | App | Size | Firm |
|---|---|---|---|---|---|---|---|
| 06.204-1 | 4.0 | 2.2 | 4.3 | 2621 | 3.9 | 38.3 | 12.5 |

### Cultivar ‘07.1-608’

In certain aspects, the present invention relates to a new and distinctive short-day type strawberry cultivar designated as ‘07.1-608’. Cultivar ‘07.1-608’ is typical of short-day strawberry cultivars and produces fruit over an extended period when treated appropriately in arid, subtropical climates.

The strawberry cultivar ‘07.1-608’ originated from a cross between two unreleased germplasm accessions ‘03.65-601’ and ‘03.111-5’. The cultivar has been asexually reproduced by runners. The properties of this cultivar were found to be transmissible by such asexual reproduction. The cultivar is stable and reproduces true to type in successive generations of asexual reproduction.

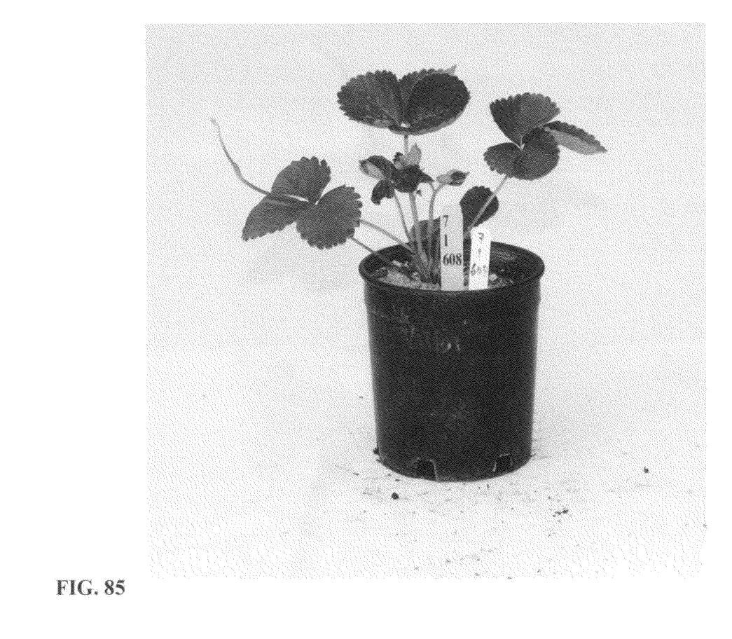

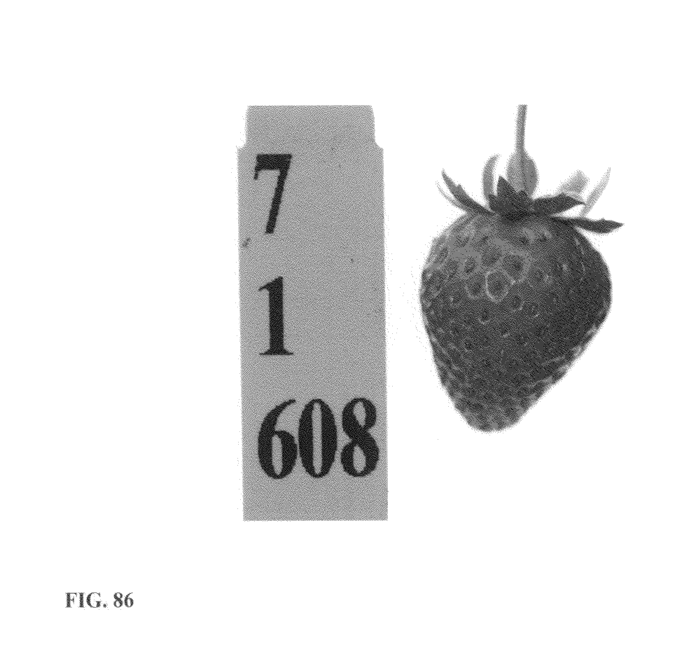

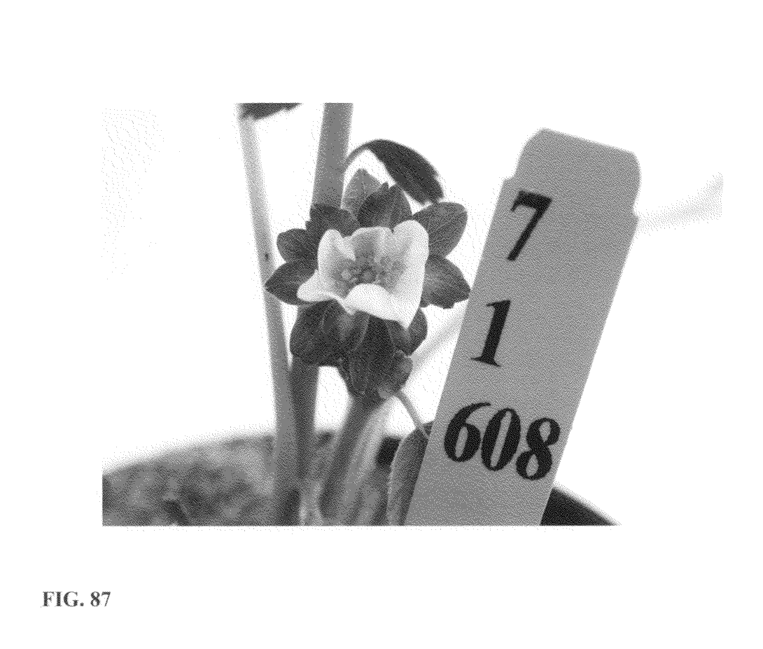

Cultivar ‘07.1-608’ has distinct disease/pathogen resistance, yield, appearance, size, and firmness. The plant of cultivar ‘07.1-608’ is characterized and distinguished in FIG. 85, the fruit of cultivar ‘07.1-608’ is characterized and distinguished in FIG. 86, and the flower of cultivar ‘07.1-608’ is characterized and distinguished in FIG. 87.

The following detailed description of cultivar ‘07.1-608’ is based on observations taken of plants and fruits (see FIGS. 85 and 86).

Certain characteristics of cultivar ‘07.1-608’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 29 provides data that characterizes the disease/pathogen resistance, yield, appearance, size, and firmness of cultivar ‘07.1-608’.

TABLE 29

| Cultivar | Vert | Phy | Fus | Yield | App | Size | Firm |
|---|---|---|---|---|---|---|---|
| 07.1-608 | 2.5 | 2.3 | 5.0 | 2274 | 3.8 | 34.0 | 13.3 |

### Cultivar ‘07.9-601’

In certain aspects, the present invention relates to a new and distinctive short-day type strawberry cultivar designated as ‘07.9-601’. Cultivar ‘07.9-601’ is typical of short-day strawberry cultivars and produces fruit over an extended period when treated appropriately in arid, subtropical climates.

The strawberry cultivar ‘07.9-601’ originated from a cross between two unreleased germplasm accessions ‘04.38-603’ and ‘04.73-2’. The cultivar has been asexually reproduced by runners. The properties of this cultivar were found to be transmissible by such asexual reproduction. The cultivar is stable and reproduces true to type in successive generations of asexual reproduction.

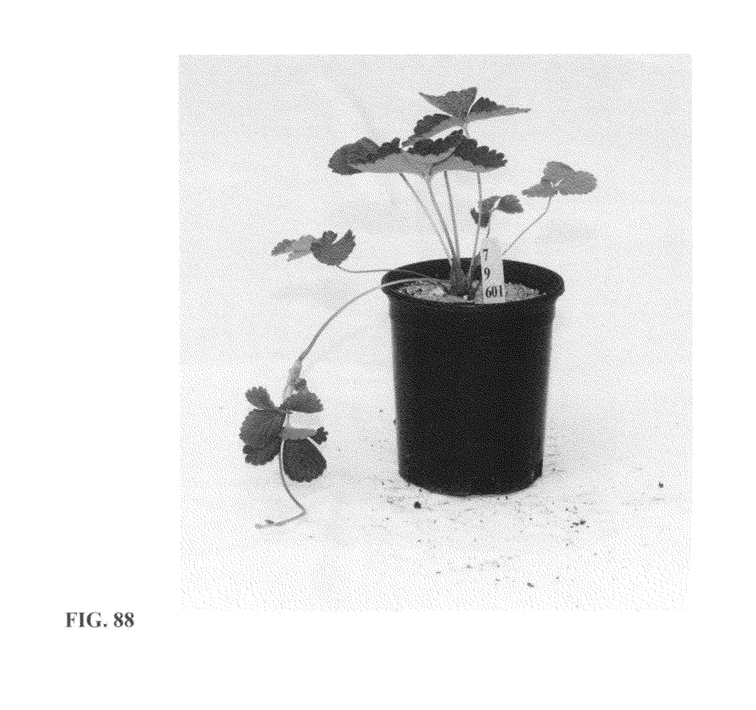

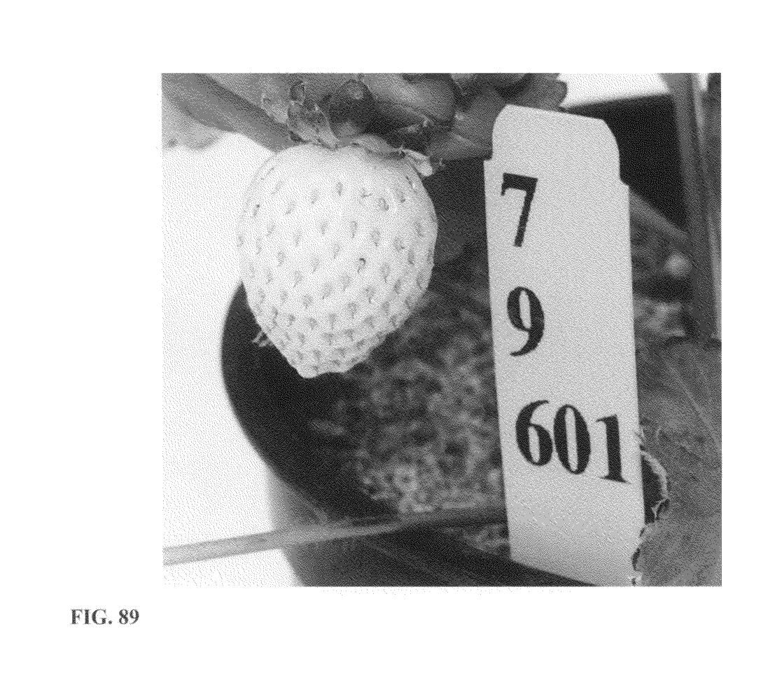

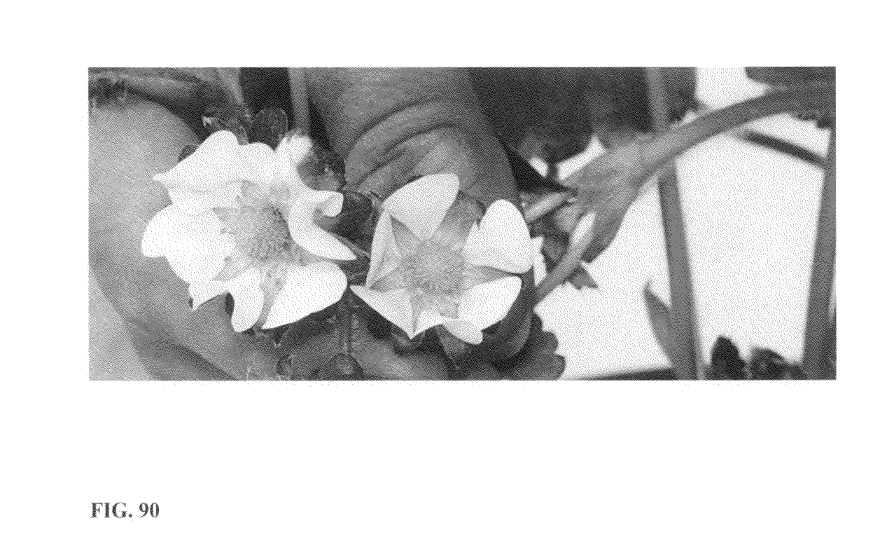

Cultivar ‘07.9-601’ has distinct disease/pathogen resistance, yield, appearance, size, and firmness. The plant of cultivar ‘07.9-601’ is characterized and distinguished in FIG. 88, and the fruit of cultivar ‘07.9-601’ is characterized and distinguished in FIG. 89, and the flower of cultivar ‘07.9-601’ is characterized and distinguished in FIG. 90.

The following detailed description of cultivar ‘07.9-601’ is based on observations taken of plants and fruits (see FIGS. 89 and 90).

Certain characteristics of cultivar ‘07.9-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 30 provides data that characterizes the disease/pathogen resistance, yield, appearance, size, and firmness of cultivar ‘07.9-601’.

TABLE 30

| Cultivar | Vert | Phy | Yield | App | Size | Firm |
|---|---|---|---|---|---|---|
| 07.9-601 | 3.5 | 3.9 | 2116 | 3.7 | 33.4 | 13.9 |

### Cultivar ‘07.64-2’

In certain aspects, the present invention relates to a new and distinctive day-neutral type strawberry cultivar designated as ‘07.64-2’. Cultivar ‘07.64-2’ is typical of day-neutral strawberry cultivars and produces fruit regardless of day length when treated appropriately in arid, subtropical climates.

The strawberry cultivar ‘07.64-2’ originated from a cross between cultivar ‘San Andreas’ (U.S. Plant Pat. No. 19,975) and unreleased germplasm accession ‘03.1-606’. The cultivar has been asexually reproduced by runners. The properties of this cultivar were found to be transmissible by such asexual reproduction. The cultivar is stable and reproduces true to type in successive generations of asexual reproduction.

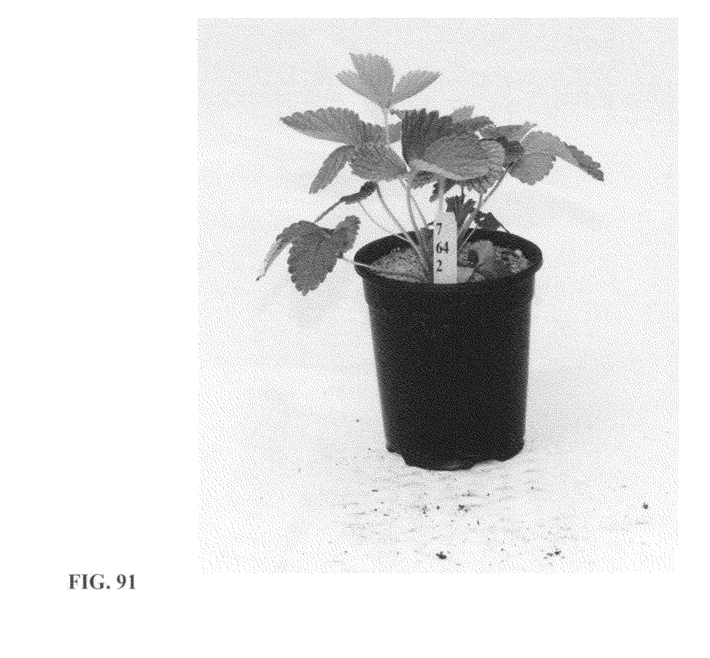

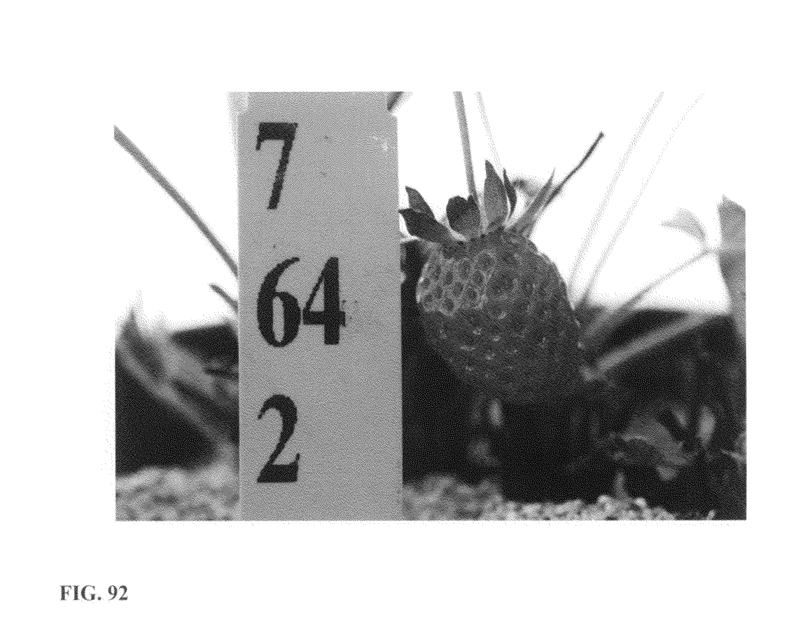

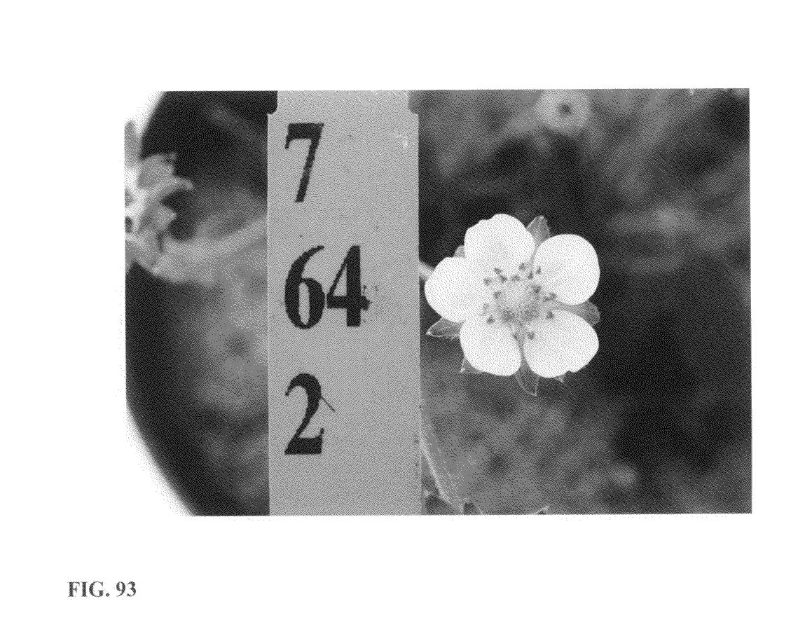

Cultivar ‘07.64-2’ has distinct disease/pathogen resistance, yield, appearance, size, and firmness. The plant of cultivar ‘07.64-2’ is characterized and distinguished in FIG. 91, the fruit of cultivar ‘07.64-2’ is characterized and distinguished in FIG. 92, and the flower of cultivar ‘07.64-2’ is characterized and distinguished in FIG. 93.

The following detailed description of cultivar ‘07.64-2’ is based on observations taken of plants and fruits (see FIGS. 91 and 92).

Certain characteristics of cultivar ‘07.64-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 31 provides data that characterizes the disease/pathogen resistance, yield, appearance, size, and firmness of cultivar ‘07.64-2’.

TABLE 31

| Cultivar | Vert | Phy | Fus | Yield | App | Size | Firm |
|---|---|---|---|---|---|---|---|
| 07.64-2 | 3.5 | 3.9 | 5.0 | 2352 | 4.1 | 36.6 | 10.4 |

### Cultivar ‘07.64-3’

In certain aspects, the present invention relates to a new and distinctive short-day type strawberry cultivar designated as ‘07.64-3’. Cultivar ‘07.64-3’ is typical of short-day strawberry cultivars and produces fruit over an extended period when treated appropriately in arid, subtropical climates.

The strawberry cultivar ‘07.64-3’ originated from a cross between cultivar ‘San Andreas’ (U.S. Plant Pat. No. 19,975) and unreleased germplasm accession ‘03.1-606’. The cultivar has been asexually reproduced by runners. The properties of this cultivar were found to be transmissible by such asexual reproduction. The cultivar is stable and reproduces true to type in successive generations of asexual reproduction.

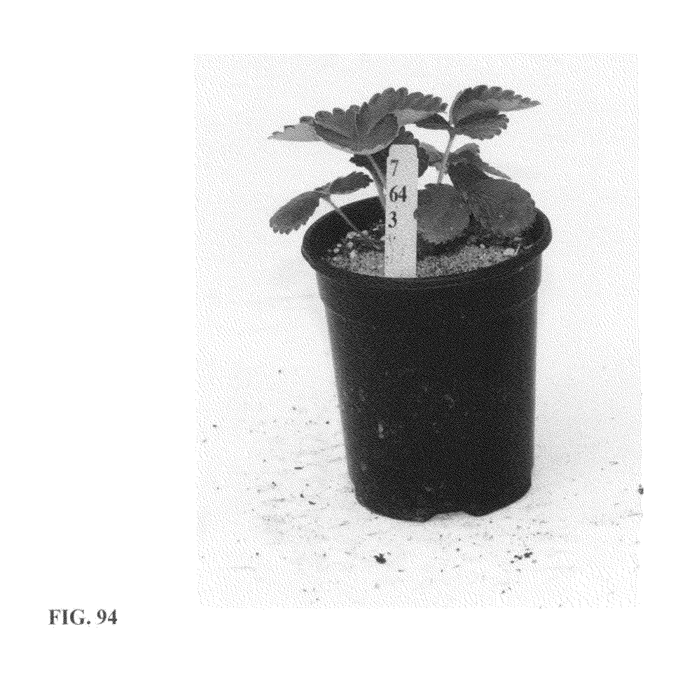

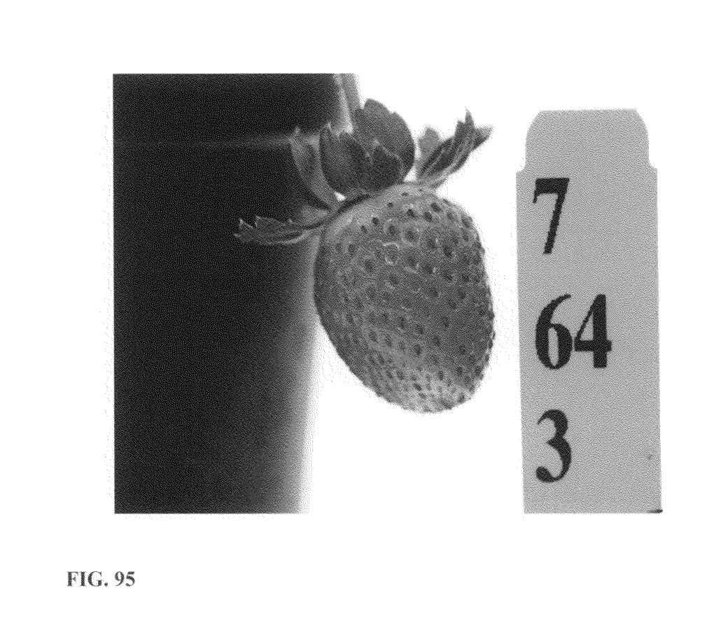

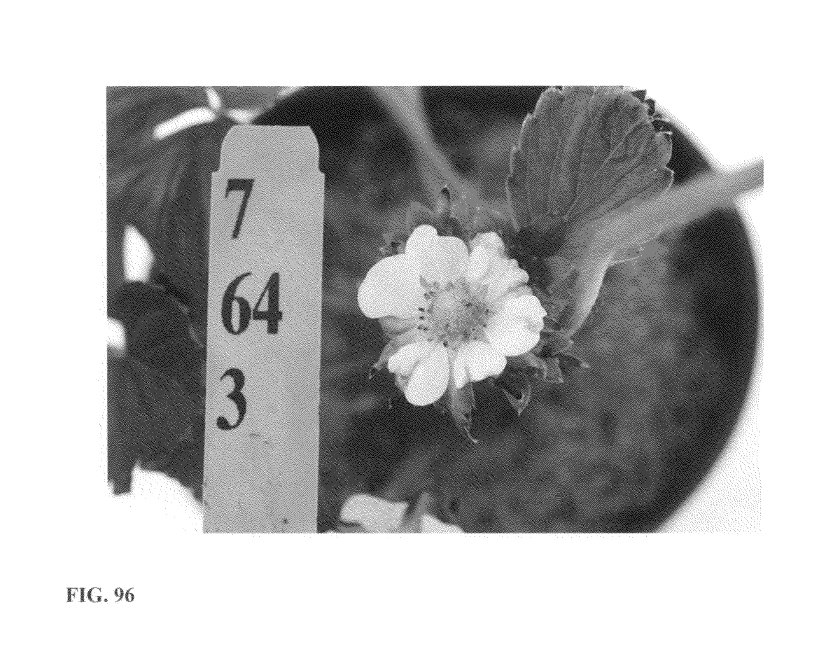

Cultivar ‘07.64-3’ has distinct disease/pathogen resistance, yield, appearance, size, and firmness. The plant of cultivar ‘07.64-3’ is characterized and distinguished in FIG. 94, the fruit of cultivar ‘07.64-3’ is characterized and distinguished in FIG. 95, and the flower of cultivar ‘07.64-3’ is characterized and distinguished in FIG. 96.

The following detailed description of cultivar ‘07.64-3’ is based on observations taken of plants and fruits (see FIGS. 94 and 95).

Certain characteristics of cultivar ‘07.64-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 32 provides data that characterizes the disease/pathogen resistance, yield, appearance, size, and firmness of cultivar ‘07.64-3’.

TABLE 32

| Cultivar | Vert | Phy | Fus | Yield | App | Size | Firm |
|---|---|---|---|---|---|---|---|
| 07.64-3 | 3.3 | 2.9 | 5.0 | 2103 | 4.1 | 35.7 | 11.3 |

### Cultivar ‘07.64-5’

In certain aspects, the present invention relates to a new and distinctive short-day type strawberry cultivar designated as ‘07.64-5’. Cultivar ‘07.64-5’ is typical of short-day strawberry cultivars and produces fruit over an extended period when treated appropriately in arid, subtropical climates.

The strawberry cultivar ‘07.64-5’ originated from a cross between cultivar ‘San Andreas’ (U.S. Plant Pat. No. 19,975) and unreleased germplasm accession ‘03.1-606’. The cultivar has been asexually reproduced by runners. The properties of this cultivar were found to be transmissible by such asexual reproduction. The cultivar is stable and reproduces true to type in successive generations of asexual reproduction.

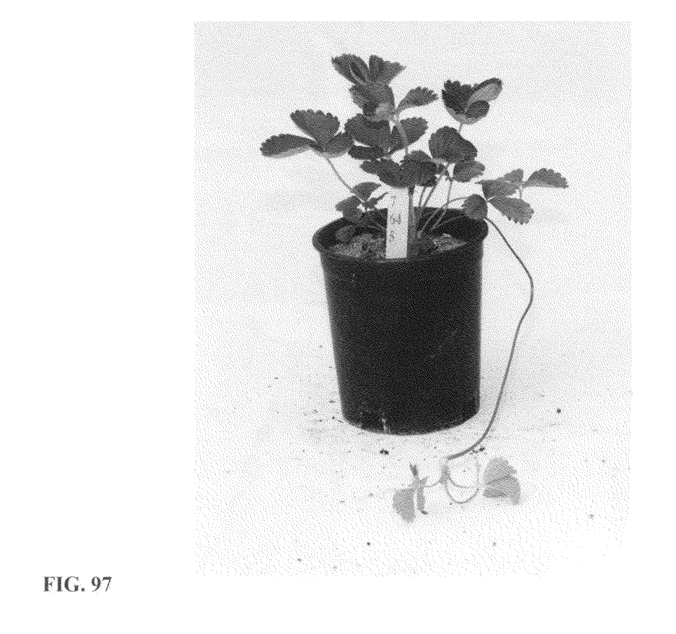

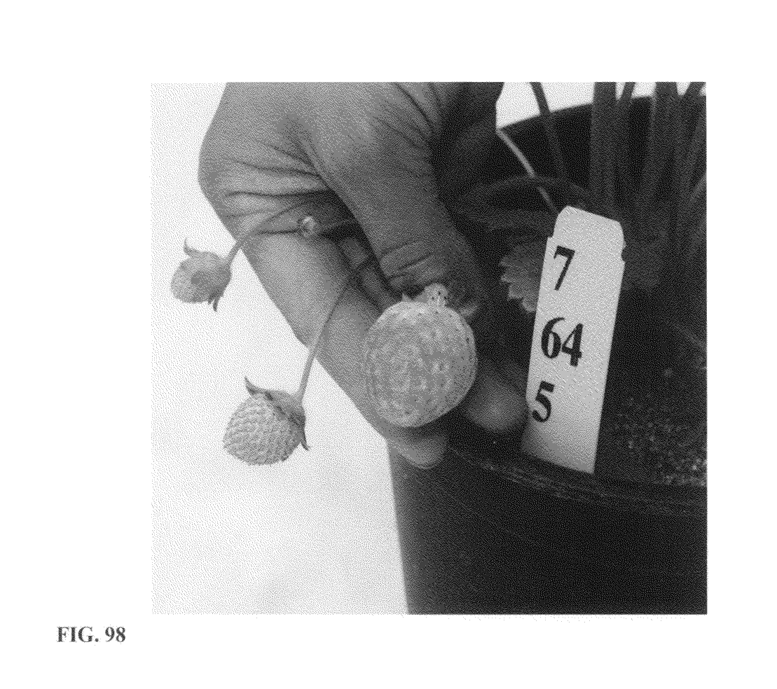

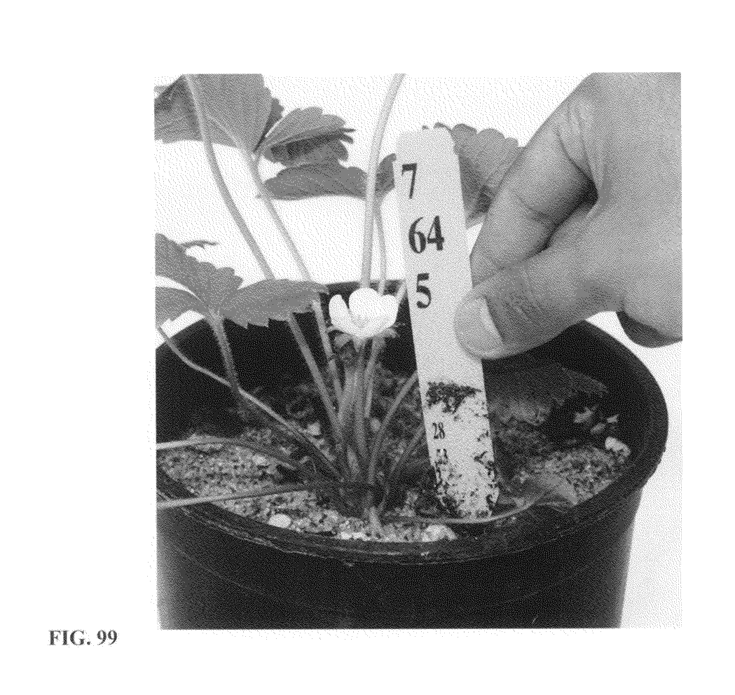

Cultivar ‘07.64-5’ has distinct disease/pathogen resistance, yield, appearance, size, and firmness. The plant of cultivar ‘07.64-5’ is characterized and distinguished in FIG. 97, the fruit of cultivar ‘07.64-5’ is characterized and distinguished in FIG. 98, and the flower of cultivar ‘07.64-5’ is characterized and distinguished in FIG. 99.

The following detailed description of cultivar ‘07.64-5’ is based on observations taken of plants and fruits (see FIGS. 97 and 98).

Certain characteristics of cultivar ‘07.64-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 33 provides data that characterizes the disease/pathogen resistance, yield, appearance, size, and firmness of cultivar ‘07.64-5’.

TABLE 33

| Cultivar | Vert | Phy | Fus | Yield | App | Size | Firm |
|---|---|---|---|---|---|---|---|
| 07.64-5 | 3.1 | 4.0 | 3.4 | 2676 | 3.6 | 35.6 | 12.0 |

### Cultivar ‘07.67-608’

In certain aspects, the present invention relates to a new and distinctive short-day type strawberry cultivar designated as ‘07.67-608’. Cultivar ‘07.67-608’ is typical of short-day strawberry cultivars and produces fruit over an extended period when treated appropriately in arid, subtropical climates.

The strawberry cultivar ‘07.67-608’ originated from a cross between two unreleased germplasm accessions ‘03.132-3’ and ‘03.1-606’. The cultivar has been asexually reproduced by runners. The properties of this cultivar were found to be transmissible by such asexual reproduction. The cultivar is stable and reproduces true to type in successive generations of asexual reproduction.

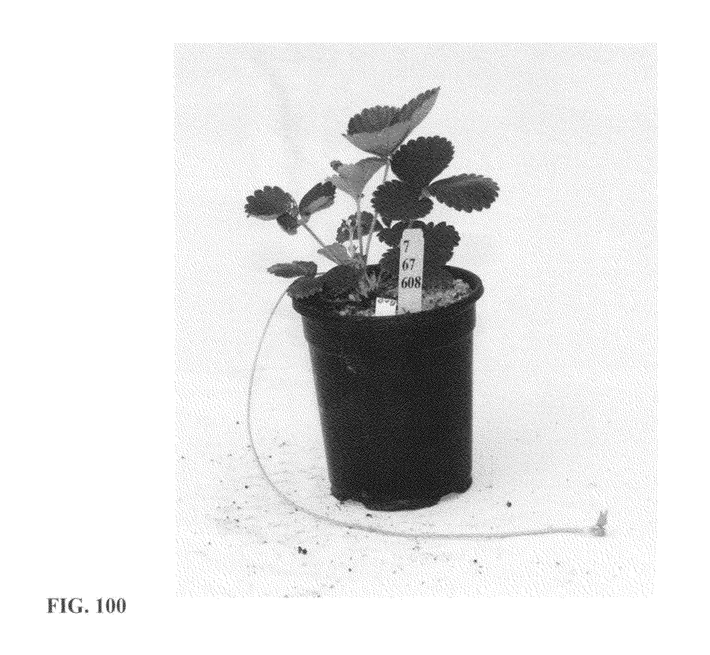

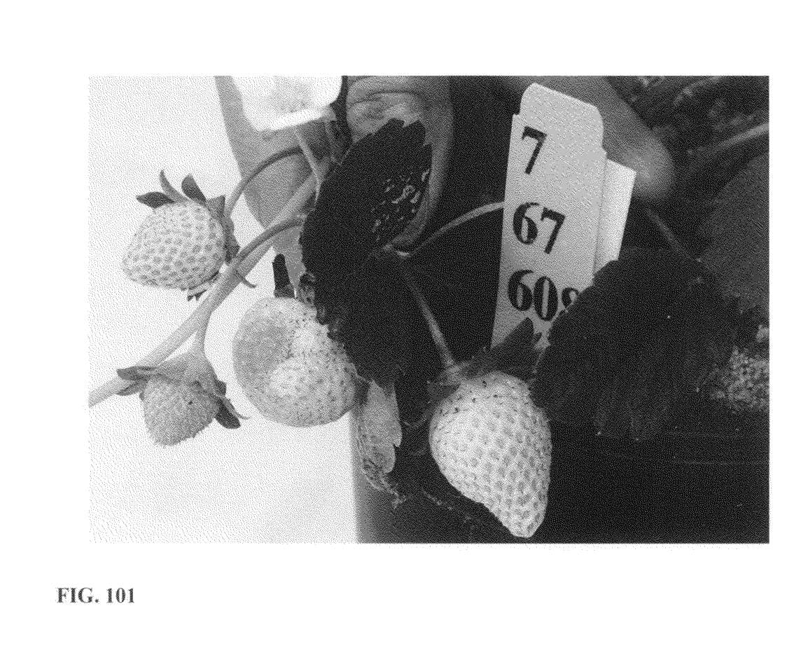

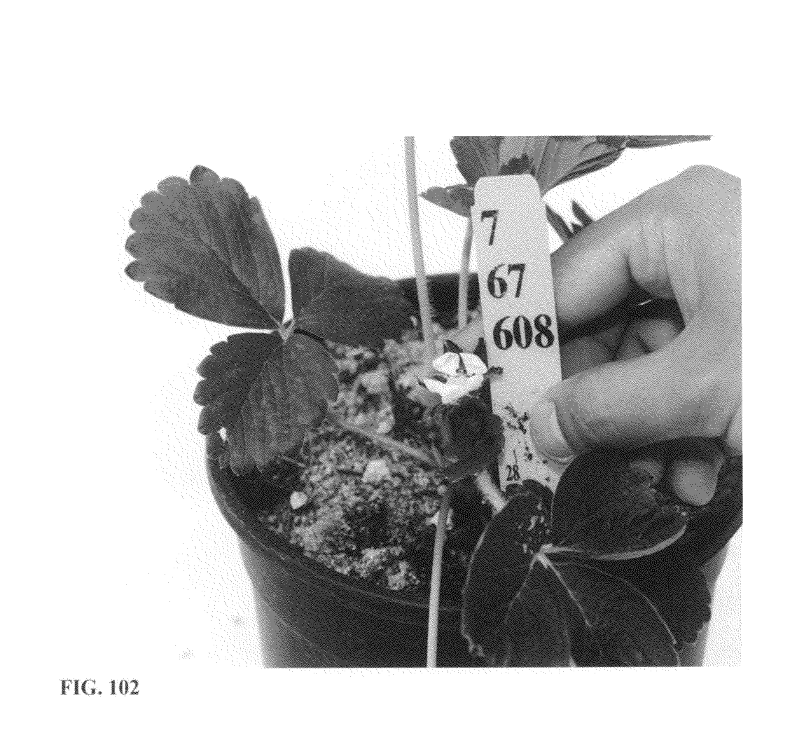

Cultivar ‘07.67-608’ has distinct disease/pathogen resistance, yield, appearance, size, and firmness. The plant of cultivar ‘07.67-608’ is characterized and distinguished in FIG. 100, the fruit of cultivar ‘07.67-608’ is characterized and distinguished in FIG. 101, and the flower of cultivar ‘07.67-608’ is characterized and distinguished in FIG. 102.

The following detailed description of cultivar ‘07.67-608’ is based on observations taken of plants and fruits (see FIGS. 100 and 101).

Certain characteristics of cultivar ‘07.67-608’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 34 provides data that characterizes the disease/pathogen resistance, yield, appearance, size, and firmness of cultivar ‘07.67-608’.

TABLE 34

| Cultivar | Vert | Phy | Yield | App | Size | Firm |
|---|---|---|---|---|---|---|
| 07.67-608 | 3.3 | 4.0 | 2612 | 3.5 | 38.0 | 12.5 |

### Cultivar ‘07.90-606’

In certain aspects, the present invention relates to a new and distinctive short-day type strawberry cultivar designated as ‘07.90-606’. Cultivar ‘07.90-606’ is typical of short-day strawberry cultivars and produces fruit over an extended period when treated appropriately in arid, subtropical climates.

The strawberry cultivar ‘07.90-606’ originated from a cross between two unreleased germplasm accessions ‘04.13-604’ and ‘04.56-608’. The cultivar has been asexually reproduced by runners. The properties of this cultivar were found to be transmissible by such asexual reproduction. The cultivar is stable and reproduces true to type in successive generations of asexual reproduction.

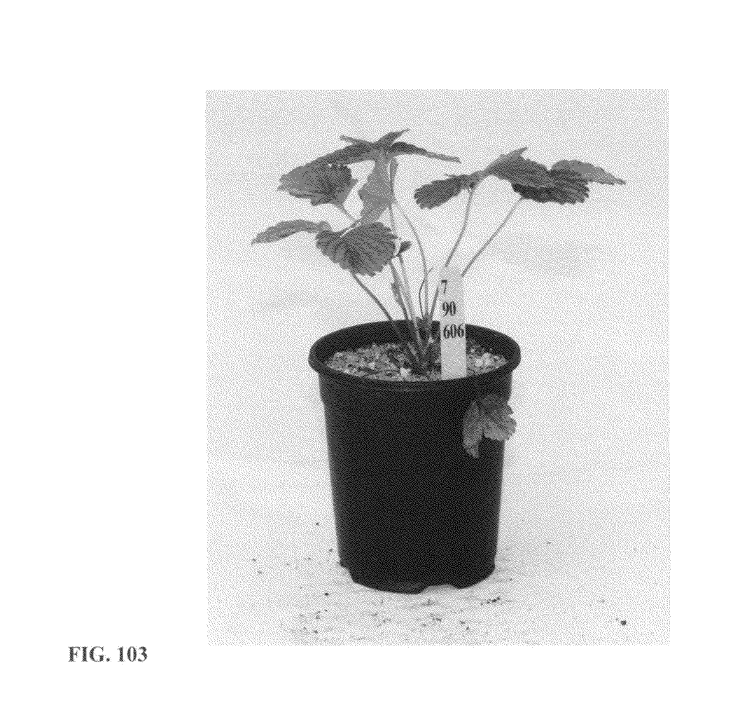

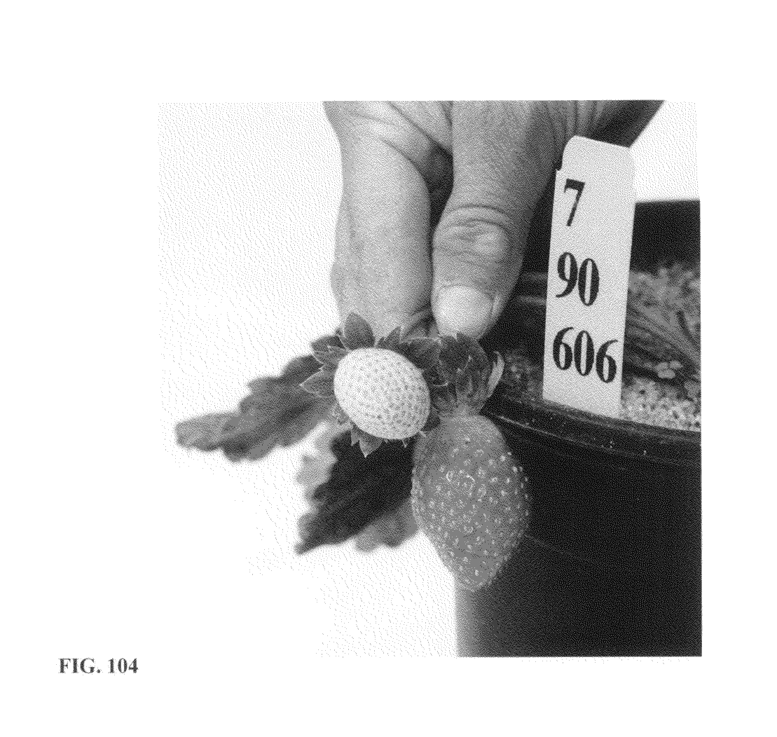

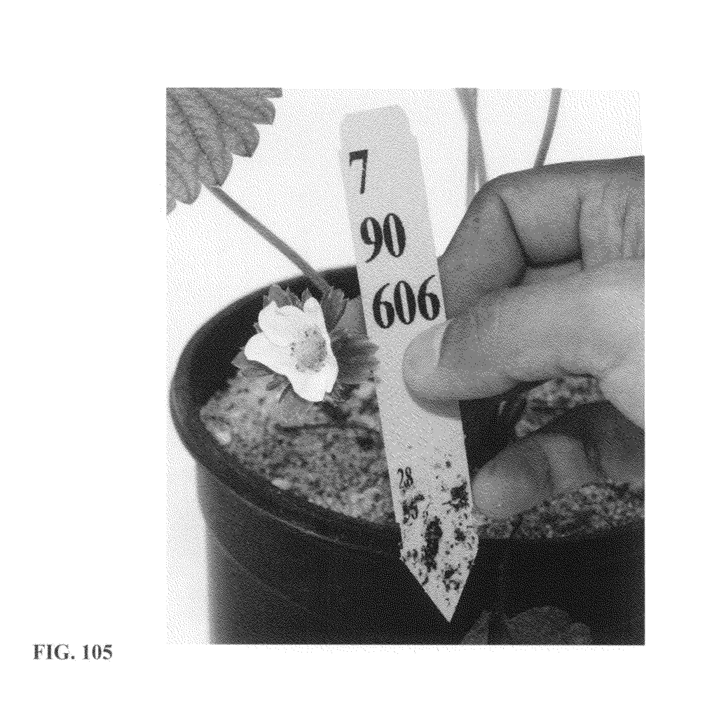

Cultivar ‘07.90-606’ has distinct disease/pathogen resistance, yield, appearance, size, and firmness. The plant of cultivar ‘07.90-606’ is characterized and distinguished in FIG. 103, the fruit of cultivar ‘07.90-606’ is characterized and distinguished in FIG. 104, and the flower of cultivar ‘07.90-606’ is characterized and distinguished in FIG. 105.

The following detailed description of cultivar ‘07.90-606’ is based on observations taken of plants and fruits (see FIGS. 103 and 104).

Certain characteristics of cultivar ‘07.90-606’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 35 provides data that characterizes the disease/pathogen resistance, yield, appearance, size, and firmness of cultivar ‘07.90-606’.

TABLE 35

| Cultivar | Vert | Yield | App | Size | Firm |
|---|---|---|---|---|---|
| 07.90-606 | 3.8 | 2210 | 3.5 | 34.5 | 12.0 |

### Cultivar ‘07.92-3’

In certain aspects, the present invention relates to a new and distinctive day-neutral type strawberry cultivar designated as ‘07.92-3’. Cultivar ‘07.92-3’ is typical of day-neutral strawberry cultivars and produces fruit regardless of day length when treated appropriately in arid, subtropical climates.

The strawberry cultivar ‘07.92-3’ originated from a cross between cultivar ‘San Andreas’ (U.S. Plant Pat. No. 19,975) and unreleased germplasm accession ‘04.73-2’. The cultivar has been asexually reproduced by runners. The properties of this cultivar were found to be transmissible by such asexual reproduction. The cultivar is stable and reproduces true to type in successive generations of asexual reproduction.

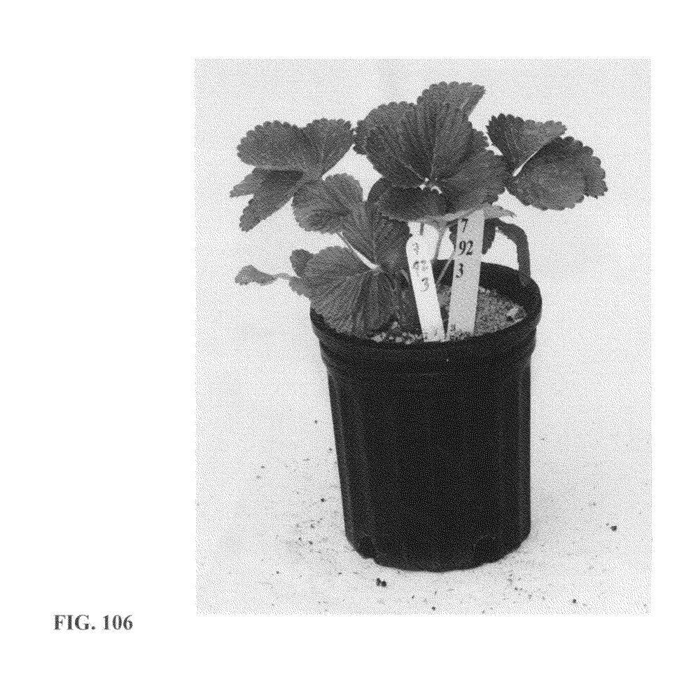

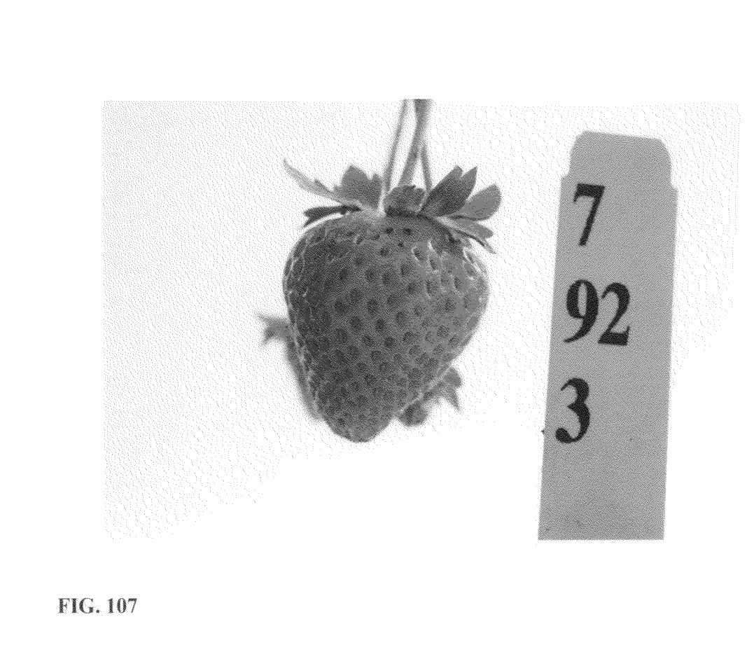

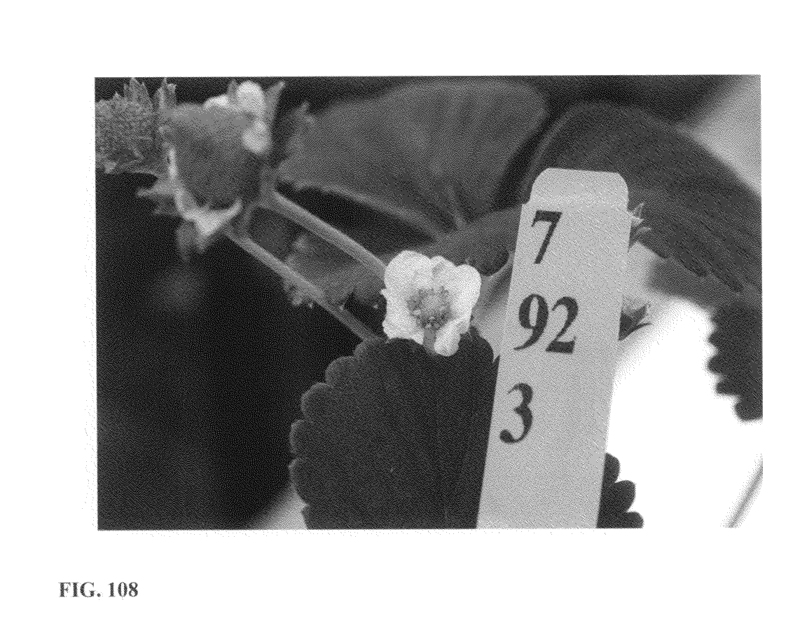

Cultivar ‘07.92-3’ has distinct disease/pathogen resistance, yield, appearance, size, and firmness. The plant of cultivar ‘07.92-3’ is characterized and distinguished in FIG. 106, the fruit of cultivar ‘07.92-3’ is characterized and distinguished in FIG. 107, and the flower of cultivar ‘07.92-3’ is characterized and distinguished in FIG. 108.

The following detailed description of cultivar ‘07.92-3’ is based on observations taken of plants and fruits (see FIGS. 106 and 107).

Certain characteristics of cultivar ‘07.92-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 36 provides data that characterizes the disease/pathogen resistance, yield, appearance, size, and firmness of cultivar ‘07.92-3’.

TABLE 36

| Cultivar | Vert | Phy | Fus | Yield | App | Size | Firm |
|---|---|---|---|---|---|---|---|
| 07.92-3 | 3.9 | 4.2 | 4.8 | 2709 | 3.7 | 40.3 | 12.5 |

### Cultivar ‘07.120-3’

In certain aspects, the present invention relates to a new and distinctive short-day type strawberry cultivar designated as ‘07.120-3’. Cultivar ‘07.120-3’ is typical of short-day strawberry cultivars and produces fruit over an extended period when treated appropriately in arid, subtropical climates.

The strawberry cultivar ‘07.120-3’ originated from a cross between two unreleased germplasm accessions ‘03.92-8’ and ‘03.149-8’. The cultivar has been asexually reproduced by runners. The properties of this cultivar were found to be transmissible by such asexual reproduction. The cultivar is stable and reproduces true to type in successive generations of asexual reproduction.

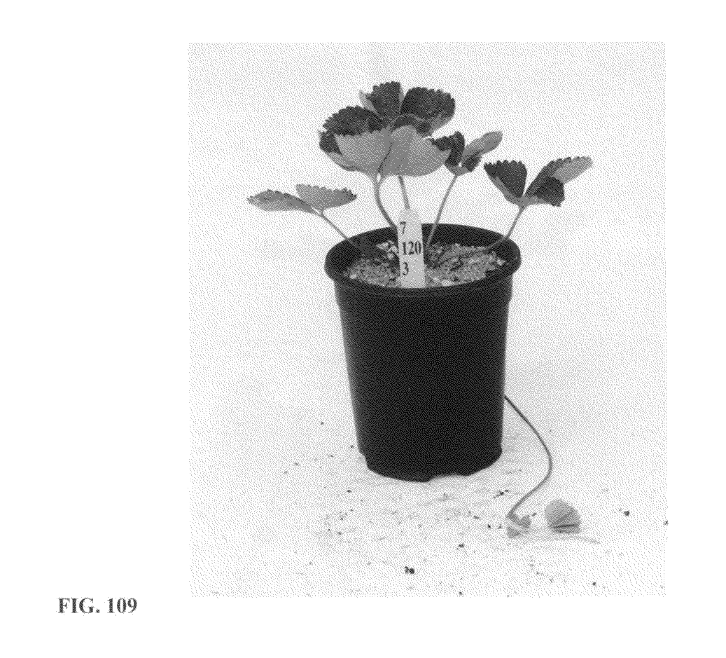

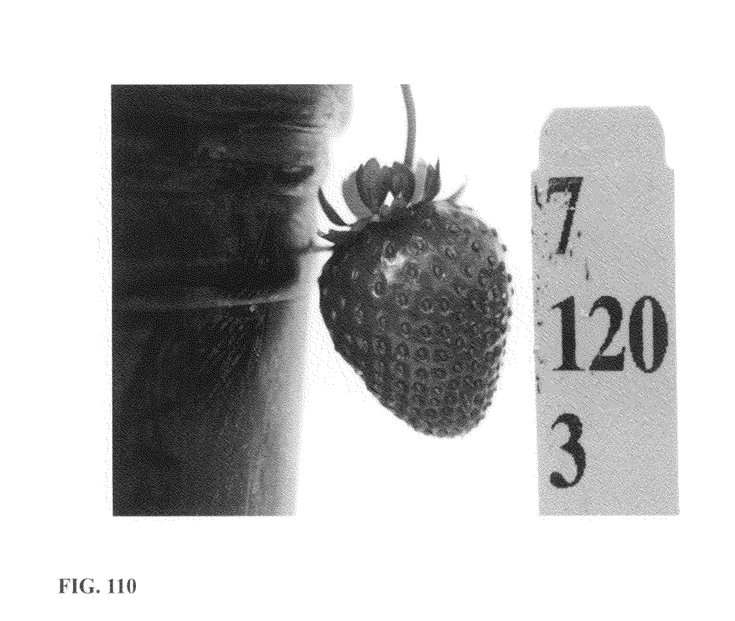

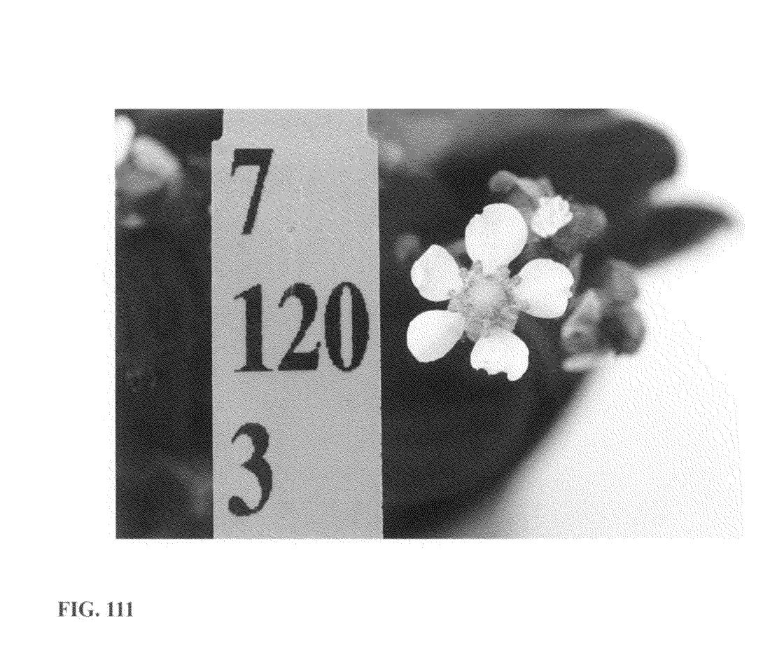

Cultivar ‘07.120-3’ has distinct disease/pathogen resistance, yield, appearance, size, and firmness. The plant of cultivar ‘07.120-3’ is characterized and distinguished in FIG. 109, the fruit of cultivar ‘07.120-3’ is characterized and distinguished in FIG. 110, and the flower of cultivar ‘07.120-3’ is characterized and distinguished in FIG. 111.

The following detailed description of cultivar ‘07.120-3’ is based on observations taken of plants and fruits (see FIGS. 109 and 110).

Certain characteristics of cultivar ‘07.120-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 37 provides data that characterizes the disease/pathogen resistance, yield, appearance, size, and firmness of cultivar ‘07.120-3’.

TABLE 37

| Cultivar | Vert | Phy | Yield | App | Size | Firm |
|---|---|---|---|---|---|---|
| 07.120-3 | 4.8 | 3.5 | 1737 | 3.8 | 34.2 | 11.9 |

### Cultivar ‘07.134-1’

In certain aspects, the present invention relates to a new and distinctive short-day type strawberry cultivar designated as ‘07.134-1’. Cultivar ‘07.134-1’ is typical of short-day strawberry cultivars and produces fruit over an extended period when treated appropriately in arid, subtropical climates.

The strawberry cultivar ‘07.134-1’ originated from a cross between cultivar ‘San Andreas’ (U.S. Plant Pat. No. 19,975) and unreleased germplasm accession ‘02.103-2’. The cultivar has been asexually reproduced by runners. The properties of this cultivar were found to be transmissible by such asexual reproduction. The cultivar is stable and reproduces true to type in successive generations of asexual reproduction.

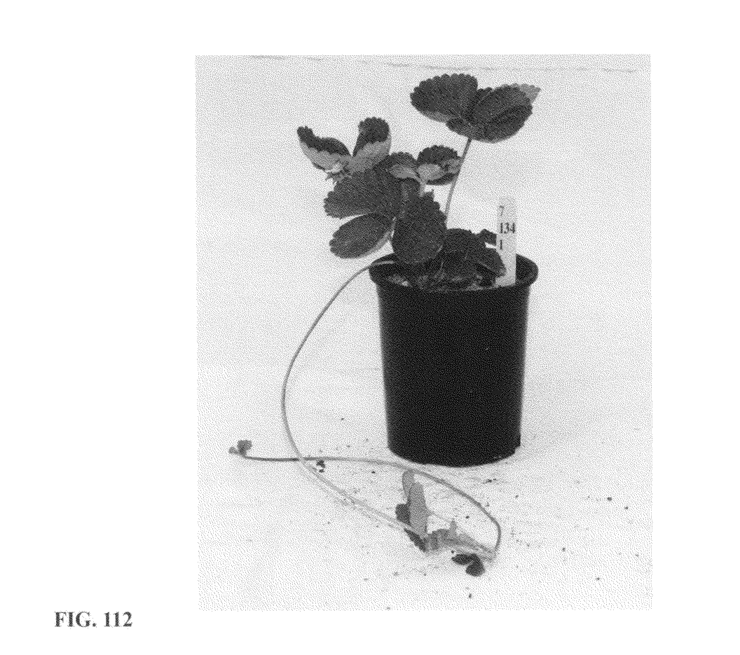

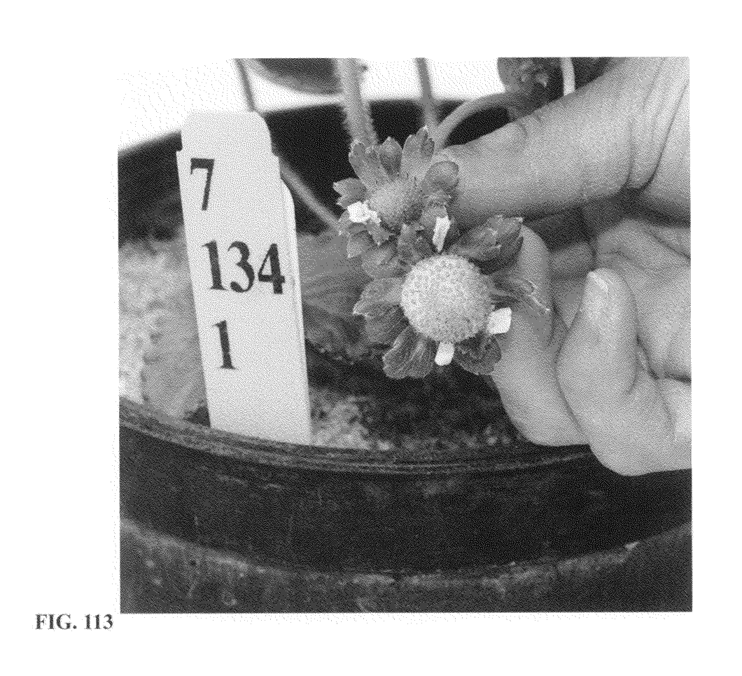

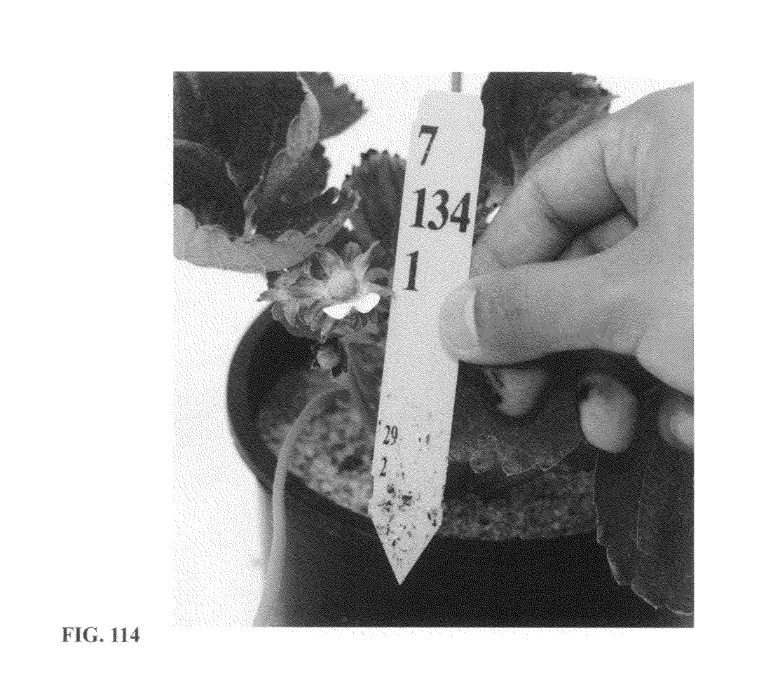

Cultivar ‘07.134-1’ has distinct disease/pathogen resistance, yield, appearance, size, and firmness. The plant of cultivar ‘07.134-1’ is characterized and distinguished in FIG. 112, the fruit of cultivar ‘07.134-1’ is characterized and distinguished in FIG. 113, and the flower of cultivar ‘07.134-1’ is characterized and distinguished in FIG. 114.

The following detailed description of cultivar ‘07.134-1’ is based on observations taken of plants and fruits (see FIGS. 112 and 113).

Certain characteristics of cultivar ‘07.134-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 38 provides data that characterizes the disease/pathogen resistance, yield, appearance, size, and firmness of cultivar ‘07.134-1’.

TABLE 38

| Cultivar | Vert | Phy | Fus | Yield | App | Size | Firm |
|---|---|---|---|---|---|---|---|
| 07.134-1 | 4.8 | 3.5 | 4.8 | 2002 | 4.4 | 46.5 | 12.7 |

### Cultivar ‘07.148-1’

In certain aspects, the present invention relates to a new and distinctive short-day type strawberry cultivar designated as ‘07.148-1’. Cultivar ‘07.148-1’ is typical of short-day strawberry cultivars and produces fruit over an extended period when treated appropriately in arid, subtropical climates.

The strawberry cultivar ‘07.148-1’ originated from a cross between cultivar ‘San Andreas’ (U.S. Plant Pat. No. 19,975) and unreleased germplasm accession ‘03.132-3’. The cultivar has been asexually reproduced by runners. The properties of this cultivar were found to be transmissible by such asexual reproduction. The cultivar is stable and reproduces true to type in successive generations of asexual reproduction.

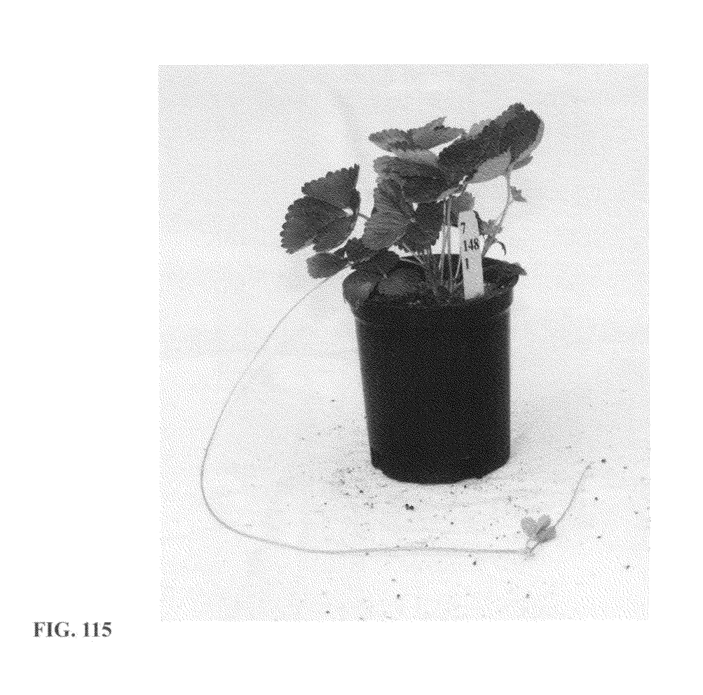

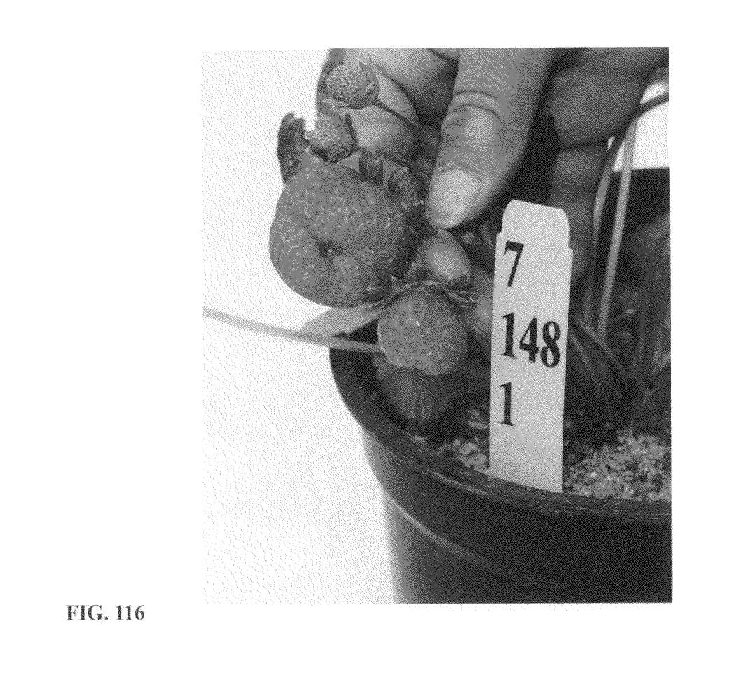

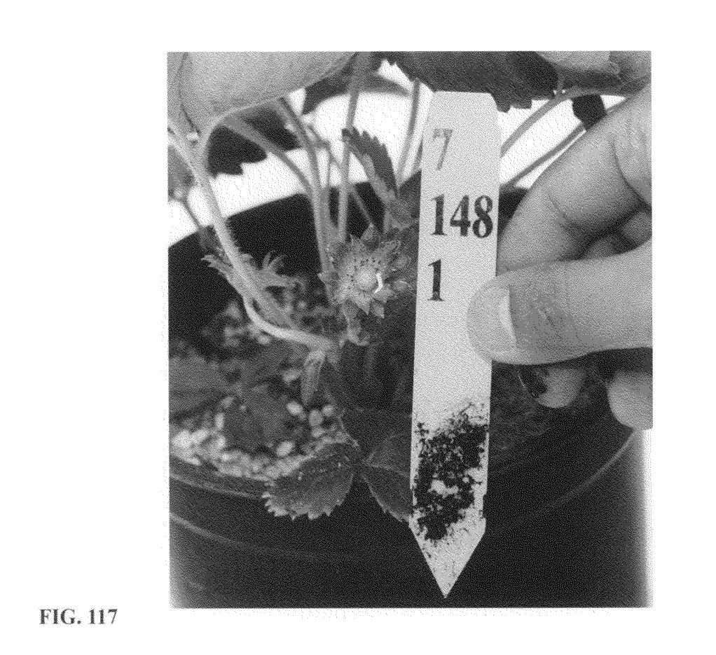

Cultivar ‘07.148-1’ has distinct disease/pathogen resistance, yield, appearance, size, and firmness. The plant of cultivar ‘07.148-1’ is characterized and distinguished in FIG. 115, the fruit of cultivar ‘07.148-1’ is characterized and distinguished in FIG. 116, and the flower of cultivar ‘07.148-1’ is characterized and distinguished in FIG. 117.

The following detailed description of cultivar ‘07.148-1’ is based on observations taken of plants and fruits (see FIGS. 115 and 116).

Certain characteristics of cultivar ‘07.148-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 39 provides data that characterizes the disease/pathogen resistance, yield, appearance, size, and firmness of cultivar ‘07.148-1’.

TABLE 39

| Cultivar | Vert | Phy | Fus | Yield | App | Size | Firm |
|---|---|---|---|---|---|---|---|
| 07.148-1 | 4.5 | 4.4 | 5.0 | 2740 | 4.0 | 34.3 | 12.5 |

### Cultivar ‘07.148-3’

In certain aspects, the present invention relates to a new and distinctive day-neutral type strawberry cultivar designated as ‘07.148-3’. Cultivar ‘07.148-3’ is typical of day-neutral strawberry cultivars and produces fruit regardless of day length when treated appropriately in arid, subtropical climates.

The strawberry cultivar ‘07.148-3’ originated from a cross between cultivar ‘San Andreas’ (U.S. Plant Pat. No. 19,975) and unreleased germplasm accession ‘03.132-3’. The cultivar has been asexually reproduced by runners. The properties of this cultivar were found to be transmissible by such asexual reproduction. The cultivar is stable and reproduces true to type in successive generations of asexual reproduction.

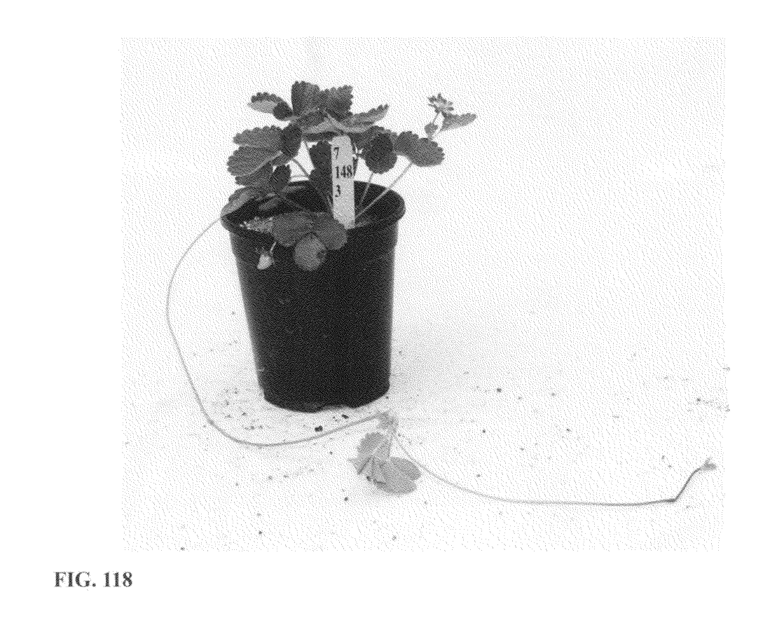

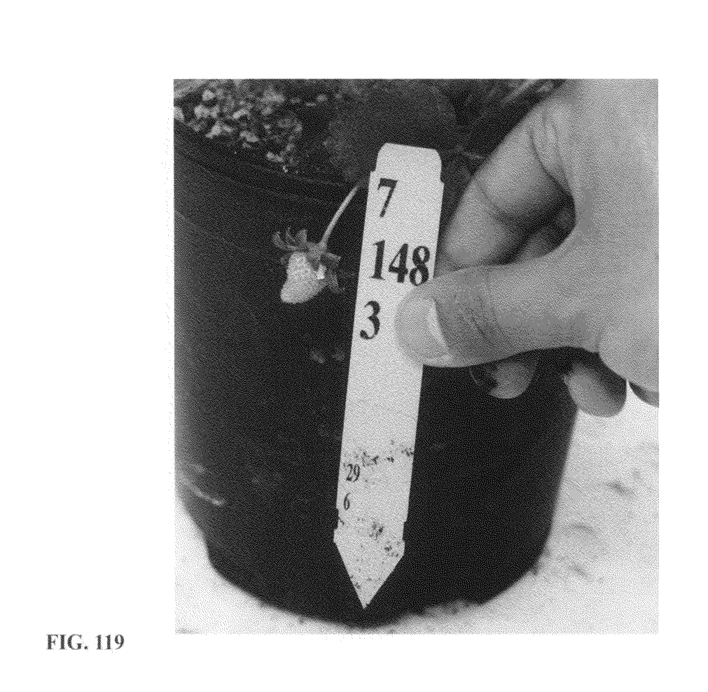

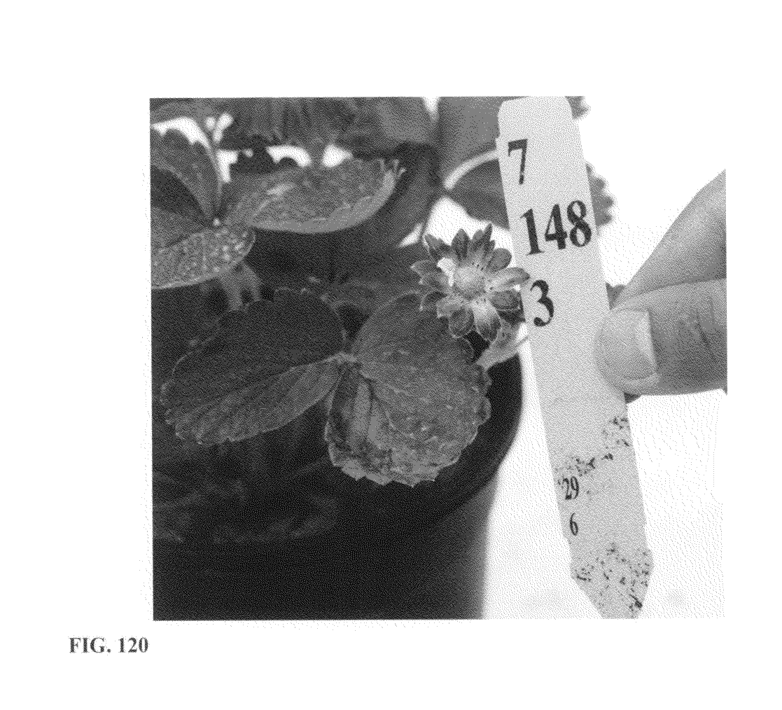

Cultivar ‘07.148-3’ has distinct disease/pathogen resistance, yield, appearance, size, and firmness. The plant of cultivar ‘07.148-3’ is characterized and distinguished in FIG. 118, the fruit of cultivar ‘07.148-3’ is characterized and distinguished in FIG. 119, and the flower of cultivar ‘07.148-3’ is characterized and distinguished in FIG. 120.

The following detailed description of cultivar ‘07.148-3’ is based on observations taken of plants and fruits (see FIGS. 118 and 119).

Certain characteristics of cultivar ‘07.148-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 40 provides data that characterizes the disease/pathogen resistance, yield, appearance, size, and firmness of cultivar ‘07.148-3’.

TABLE 40

| Cultivar | Vert | Phy | Fus | Yield | App | Size | Firm |
|---|---|---|---|---|---|---|---|
| 07.148-3 | 4.9 | 4.1 | 5.0 | 2329 | 3.5 | 30.6 | 13.7 |

### Cultivar ‘08.5-601’

In certain aspects, the present invention relates to a new and distinctive short-day type strawberry cultivar designated as ‘08.5-601’. Cultivar ‘08.5-601’ is typical of short-day strawberry cultivars and produces fruit over an extended period when treated appropriately in arid, subtropical climates.

The strawberry cultivar ‘08.5-601’ originated from a cross between cultivar ‘Benicia’ (U.S. Plant Pat. No. 22,542) and unreleased germplasm accession ‘04.77-2’. The cultivar has been asexually reproduced by runners. The properties of this cultivar were found to be transmissible by such asexual reproduction. The cultivar is stable and reproduces true to type in successive generations of asexual reproduction.

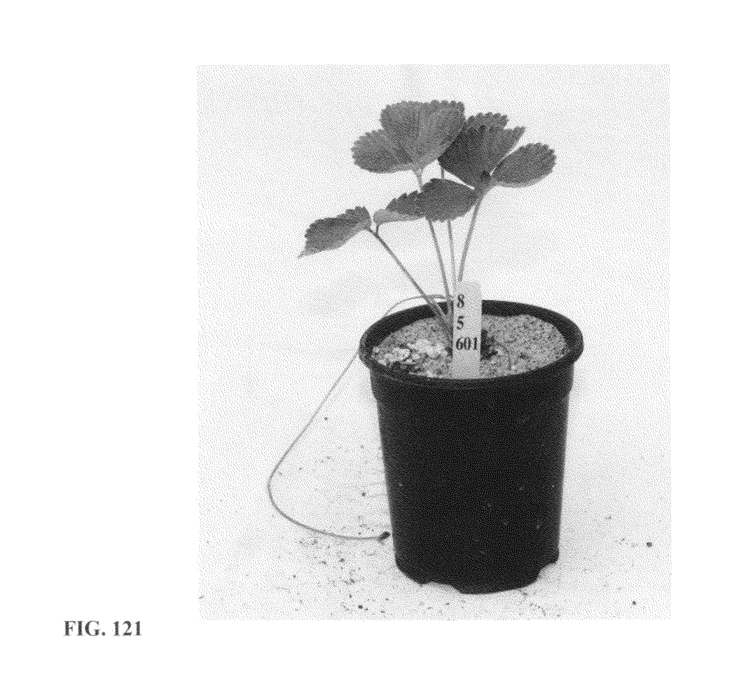

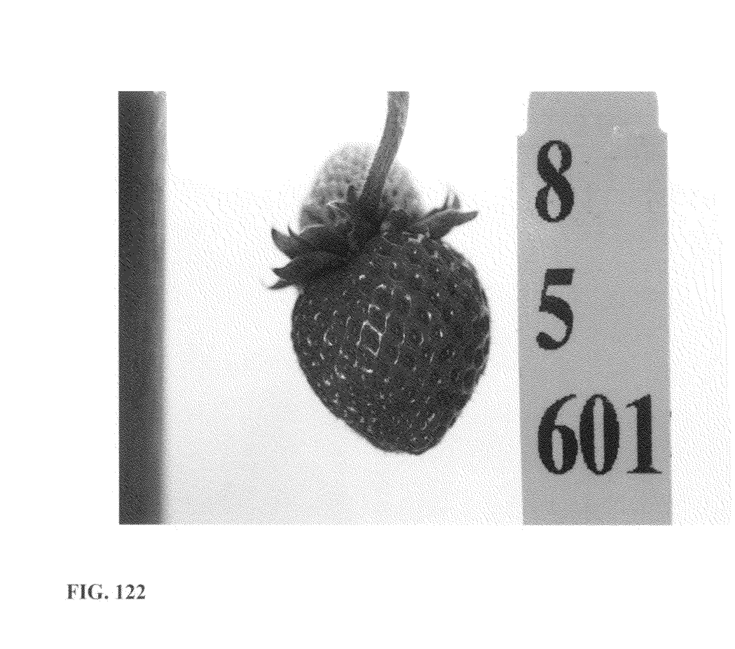

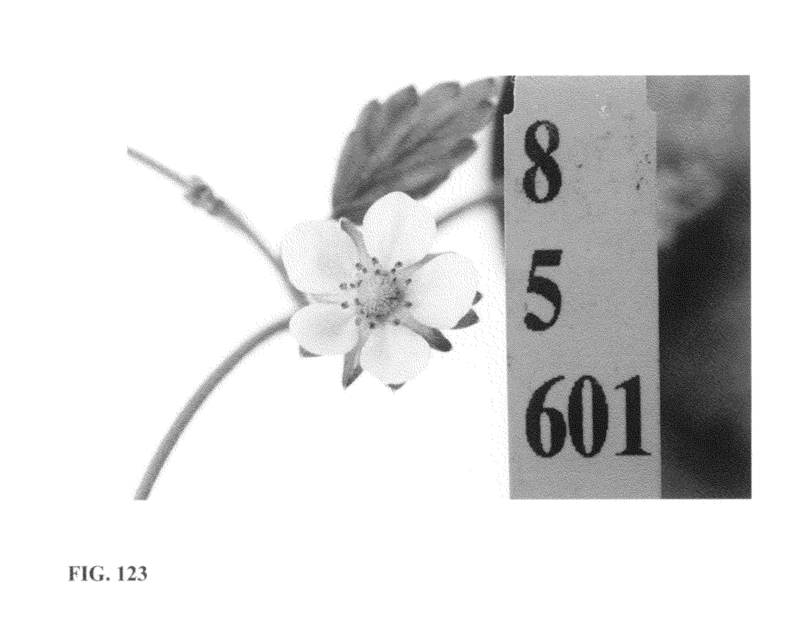

Cultivar ‘08.5-601’ has distinct disease/pathogen resistance, yield, appearance, size, and firmness. The plant of cultivar ‘08.5-601’ is characterized and distinguished in FIG. 121, the fruit of cultivar ‘08.5-601’ is characterized and distinguished in FIG. 122, and the flower of cultivar ‘08.5-601’ is characterized and distinguished in FIG. 123.

The following detailed description of cultivar ‘08.5-601’ is based on observations taken of plants and fruits (see FIGS. 121 and 122).

Certain characteristics of cultivar ‘08.5-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 41 provides data that characterizes the disease/pathogen resistance, yield, appearance, size, and firmness of cultivar ‘08.5-601’.

TABLE 41

| Cultivar | Vert | Phy | Fus | Mac | Yield | App | Size | Firm |
|---|---|---|---|---|---|---|---|---|
| 08.5-601 | 4.3 | 4.8 | 4.6 | 2.6 | 1860 | 3.4 | 30.7 | 10.9 |

### Cultivar ‘08.13-605’

In certain aspects, the present invention relates to a new and distinctive short-day type strawberry cultivar designated as ‘08.13-605’. Cultivar ‘08.13-605’ is typical of short-day strawberry cultivars and produces fruit over an extended period when treated appropriately in arid, subtropical climates.

The strawberry cultivar ‘08.13-605’ originated from a cross between two unreleased germplasm accessions ‘04.41-6’ and ‘05.117-5’. The cultivar has been asexually reproduced by runners. The properties of this cultivar were found to be transmissible by such asexual reproduction. The cultivar is stable and reproduces true to type in successive generations of asexual reproduction.

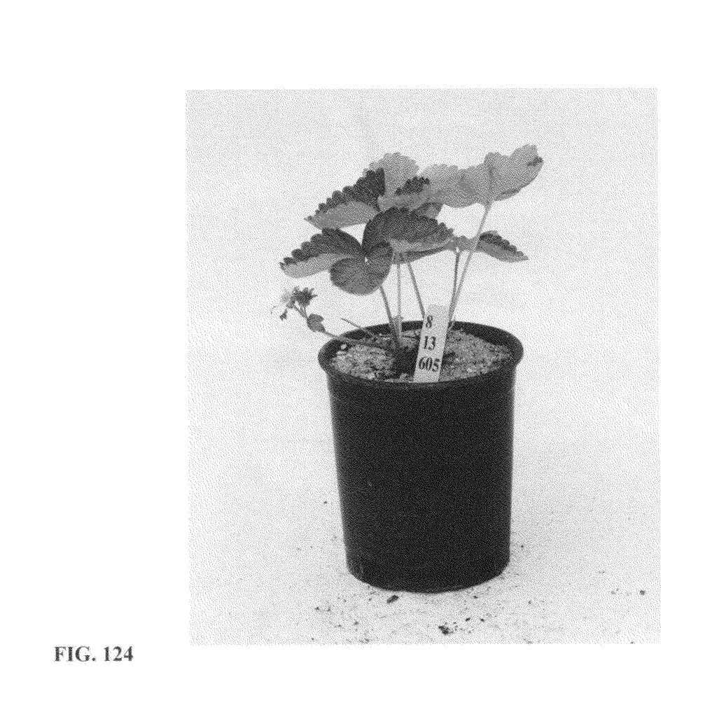

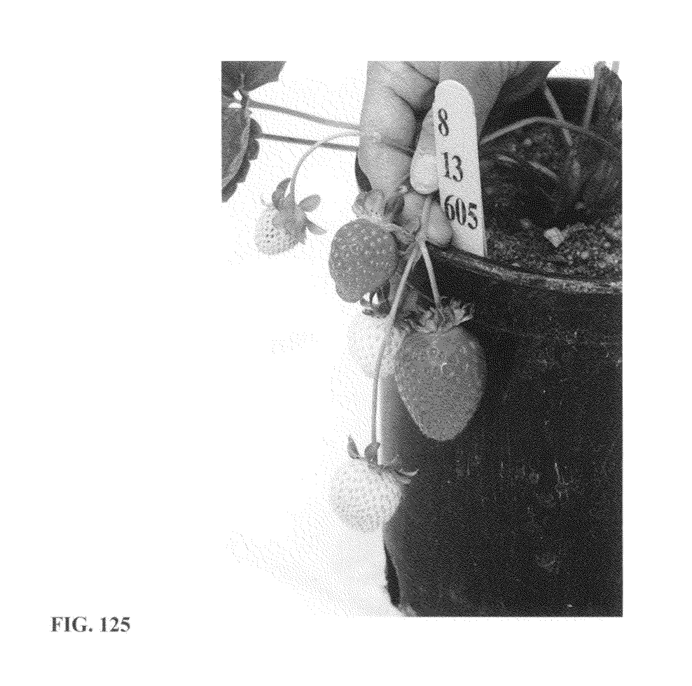

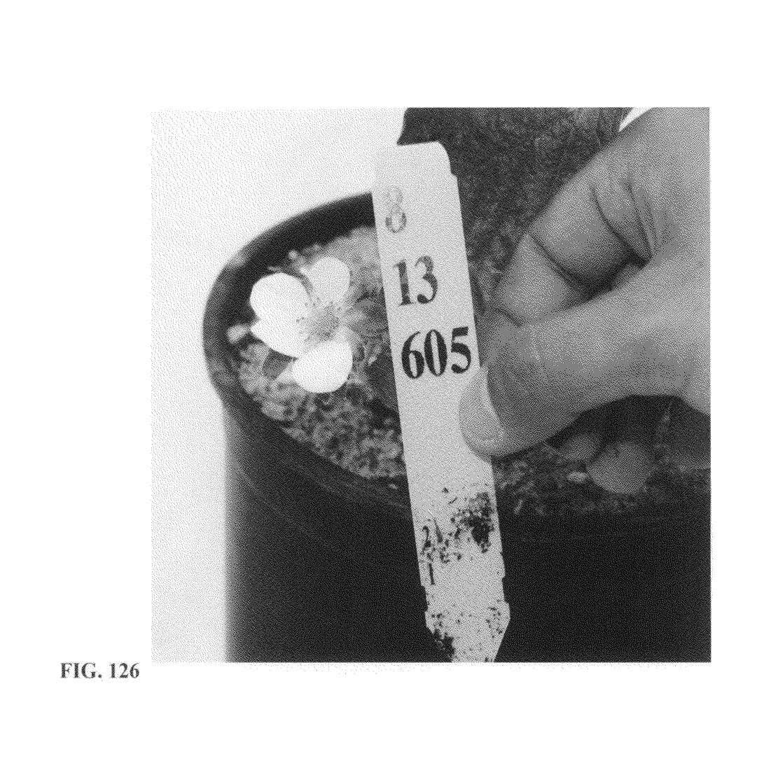

Cultivar ‘08.13-605’ has distinct disease/pathogen resistance, yield, appearance, size, and firmness. The plant of cultivar ‘08.13-605’ is characterized and distinguished in FIG. 124, the fruit of cultivar ‘08.13-605’ is characterized and distinguished in FIG. 125, and the flower of cultivar ‘08.13-605’ is characterized and distinguished in FIG. 126.

The following detailed description of cultivar ‘08.13-605’ is based on observations taken of plants and fruits (see FIGS. 124 and 125).

Certain characteristics of cultivar ‘08.13-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 42 provides data that characterizes the disease/pathogen resistance, yield, appearance, size, and firmness of cultivar ‘08.13-605’.

TABLE 42

| Cultivar | Vert | Yield | App | Size | Firm |
|---|---|---|---|---|---|
| 08.13-605 | 4.0 | 2714 | 4.0 | 33.2 | 12.0 |

### Cultivar ‘08.17-5’

In certain aspects, the present invention relates to a new and distinctive short-day type strawberry cultivar designated as ‘08.17-5’. Cultivar ‘08.17-5’ is typical of short-day strawberry cultivars and produces fruit over an extended period when treated appropriately in arid, subtropical climates.

The strawberry cultivar ‘08.17-5’ originated from a cross between cultivar ‘Benicia’ (U.S. Plant Pat. No. 22,542) and unreleased germplasm accession ‘05.165-1’. The cultivar has been asexually reproduced by runners. The properties of this cultivar were found to be transmissible by such asexual reproduction. The cultivar is stable and reproduces true to type in successive generations of asexual reproduction.

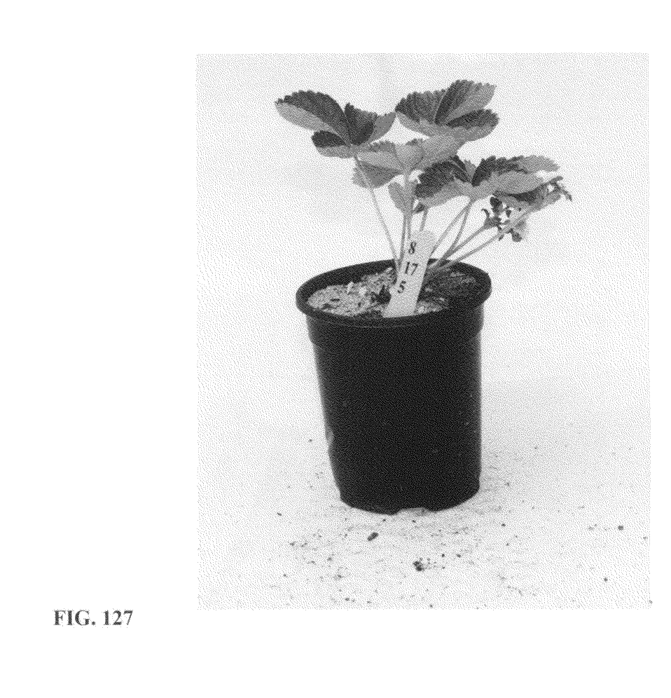

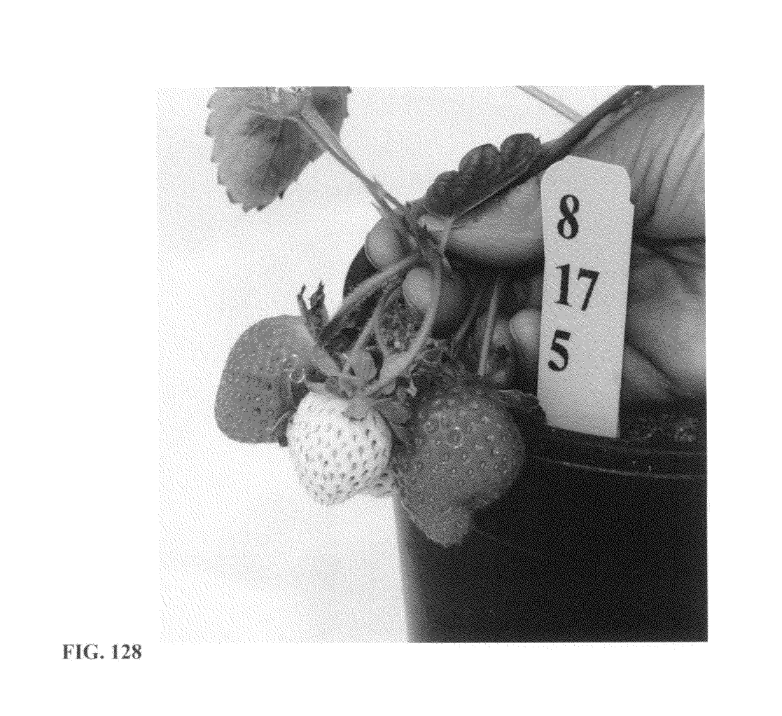

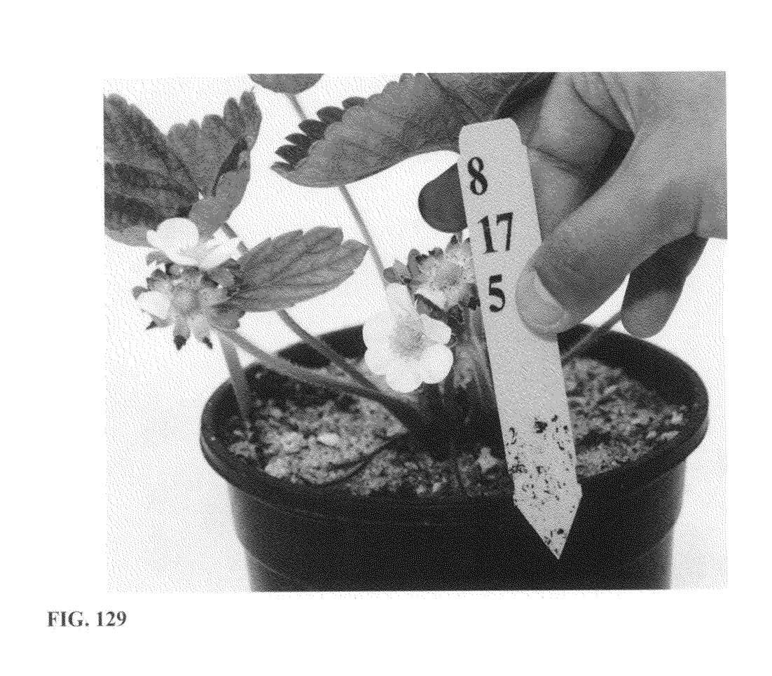

Cultivar ‘08.17-5’ has distinct disease/pathogen resistance, yield, appearance, size, and firmness. The plant of cultivar ‘08.17-5’ is characterized and distinguished in FIG. 127, the fruit of cultivar ‘08.17-5’ is characterized and distinguished in FIG. 128, and the flower of cultivar ‘08.17-5’ is characterized and distinguished in FIG. 129.

The following detailed description of cultivar ‘08.17-5’ is based on observations taken of plants and fruits (see FIGS. 127 and 128).

Certain characteristics of cultivar ‘08.17-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 43 provides data that characterizes the disease/pathogen resistance, yield, appearance, size, and firmness of cultivar ‘08.17-5’.

TABLE 43

| Cultivar | Vert | Phy | Fus | Mac | Yield | App | Size | Firm |
|---|---|---|---|---|---|---|---|---|
| 08.17-5 | 3.2 | 4.8 | 4.5 | 3.5 | 2464 | 3.8 | 33.9 | 11.3 |

### Cultivar ‘08.18-5’

In certain aspects, the present invention relates to a new and distinctive short-day type strawberry cultivar designated as ‘08.18-5’. Cultivar ‘08.18-5’ is typical of short-day strawberry cultivars and produces fruit over an extended period when treated appropriately in arid, subtropical climates.

The strawberry cultivar ‘08.18-5’ originated from a cross between cultivar ‘Benicia’ (U.S. Plant Pat. No. 22,542) and unreleased germplasm accession ‘04.77-2’. The cultivar has been asexually reproduced by runners. The properties of this cultivar were found to be transmissible by such asexual reproduction. The cultivar is stable and reproduces true to type in successive generations of asexual reproduction.

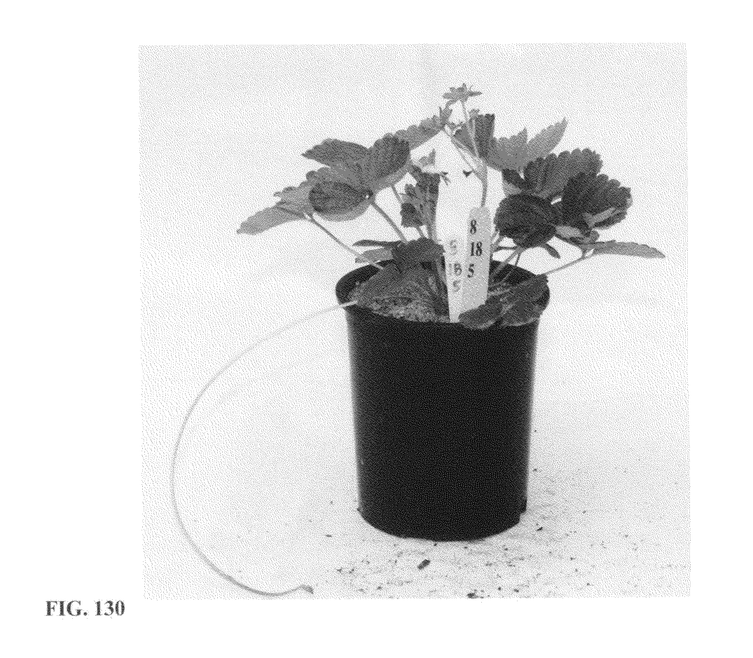

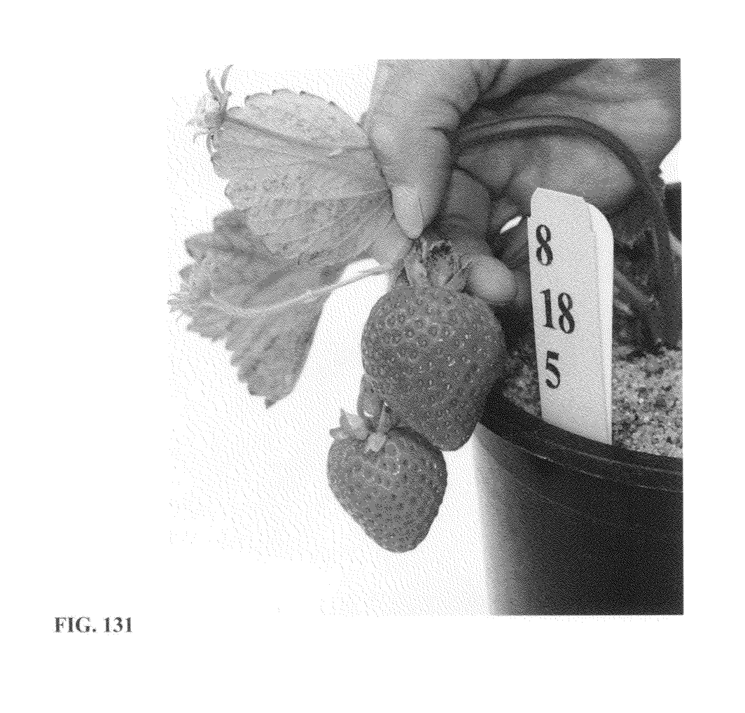

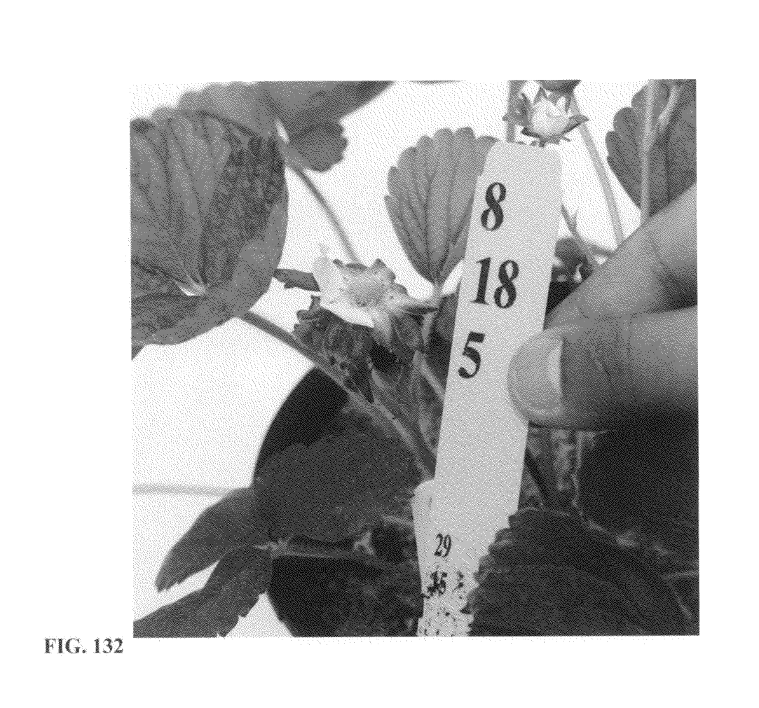

Cultivar ‘08.18-5’ has distinct disease/pathogen resistance, yield, appearance, size, and firmness. The plant of cultivar ‘08.18-5’ is characterized and distinguished in FIG. 130, the fruit of cultivar ‘08.18-5’ is characterized and distinguished in FIG. 131, and the flower of cultivar ‘08.18-5’ is characterized and distinguished in FIG. 132.

The following detailed description of cultivar ‘08.18-5’ is based on observations taken of plants and fruits (see FIGS. 130 and 131).

Certain characteristics of cultivar ‘08.18-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 44 provides data that characterizes the disease/pathogen resistance, yield, appearance, size, and firmness of cultivar ‘08.18-5’.

TABLE 44

| Cultivar | Vert | Phy | Fus | Yield | App | Size | Firm |
|---|---|---|---|---|---|---|---|
| 08.18-5 | 3.9 | 4.8 | 4.5 | 2708 | 3.4 | 39.3 | 11.0 |

### Cultivar ‘08.37-603’

In certain aspects, the present invention relates to a new and distinctive short-day type strawberry cultivar designated as ‘08.37-603’. Cultivar ‘08.37-603’ is typical of short-day strawberry cultivars and produces fruit over an extended period when treated appropriately in arid, subtropical climates.

The strawberry cultivar ‘08.37-603’ originated from a cross between two unreleased germplasm accessions ‘04.41-6’ and ‘04.1-601’. The cultivar has been asexually reproduced by runners. The properties of this cultivar were found to be transmissible by such asexual reproduction. The cultivar is stable and reproduces true to type in successive generations of asexual reproduction.

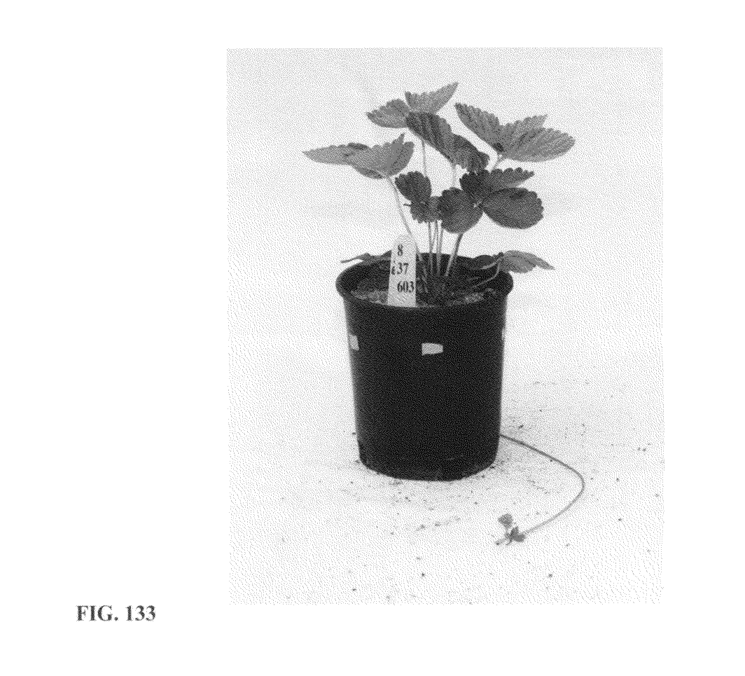

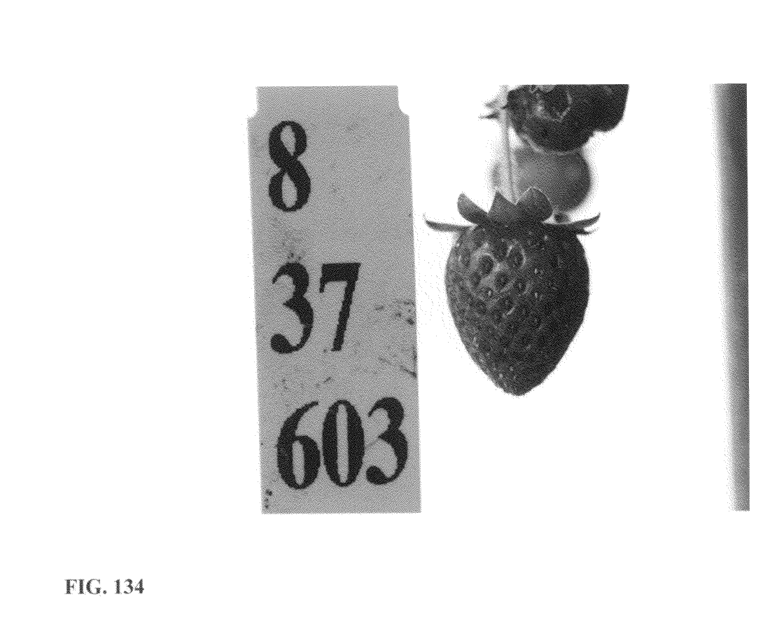

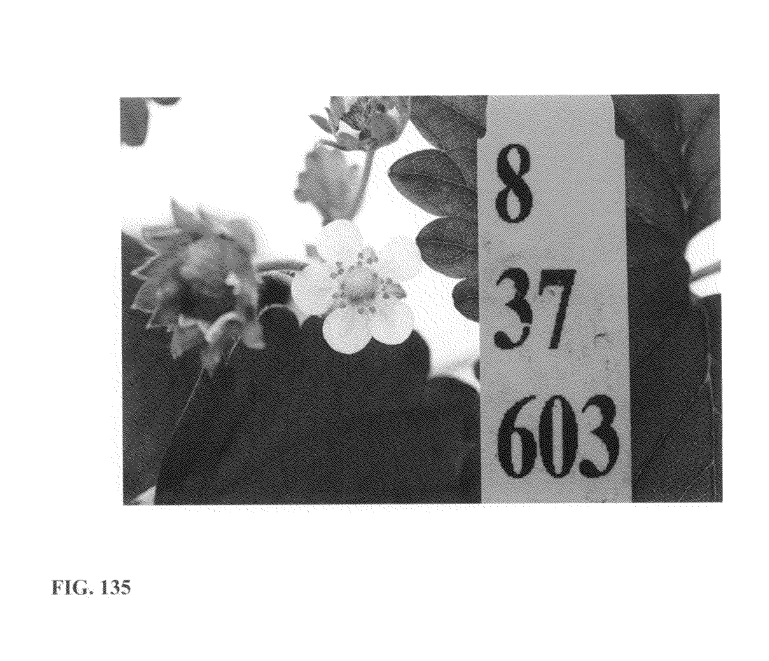

Cultivar ‘08.37-603’ has distinct disease/pathogen resistance, yield, appearance, size, and firmness. The plant of cultivar ‘08.37-603’ is characterized and distinguished in FIG. 133, the fruit of cultivar ‘08.37-603’ is characterized and distinguished in FIG. 134, and the flower of cultivar ‘08.37-603’ is characterized and distinguished in FIG. 135.

The following detailed description of cultivar ‘08.37-603’ is based on observations taken of plants and fruits (see FIGS. 133 and 134).

Certain characteristics of cultivar ‘08.37-603’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 45 provides data that characterizes the disease/pathogen resistance, yield, appearance, size, and firmness of cultivar ‘08.37-603’.

TABLE 45

| Cultivar | Vert | Phy | Fus | Mac | Yield | App | Size | Firm |
|---|---|---|---|---|---|---|---|---|
| 08.37-603 | 3.5 | 3.3 | 5.0 | 3.1 | 2081 | 3.3 | 32.4 | 12.0 |

### Cultivar ‘08.43-1’

In certain aspects, the present invention relates to a new and distinctive day-neutral type strawberry cultivar designated as ‘08.43-1’. Cultivar ‘08.43-1’ is typical of day-neutral strawberry cultivars and produces fruit regardless of day length when treated appropriately in arid, subtropical climates.

The strawberry cultivar ‘08.43-1’ originated from a cross between two unreleased germplasm accessions ‘04.41-6’ and ‘05.99-6’. The cultivar has been asexually reproduced by runners. The properties of this cultivar were found to be transmissible by such asexual reproduction. The cultivar is stable and reproduces true to type in successive generations of asexual reproduction.

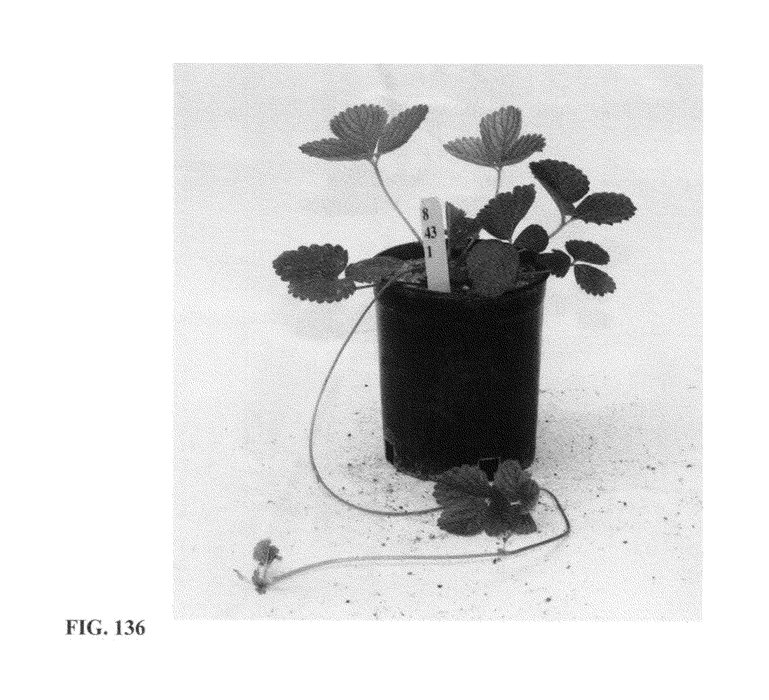

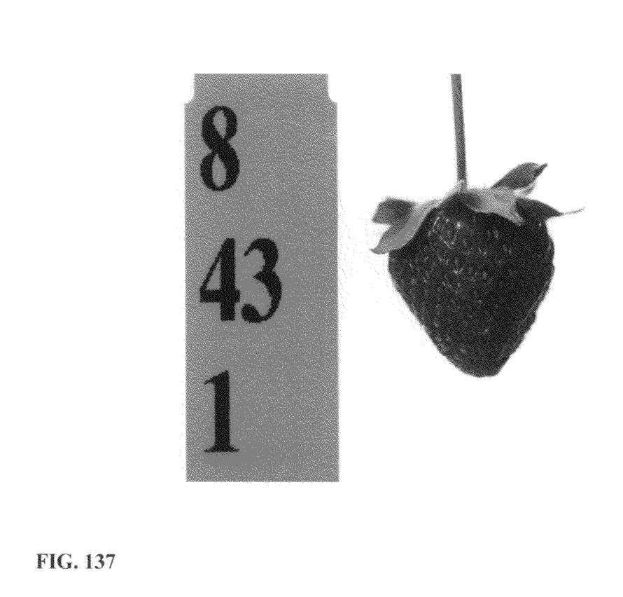

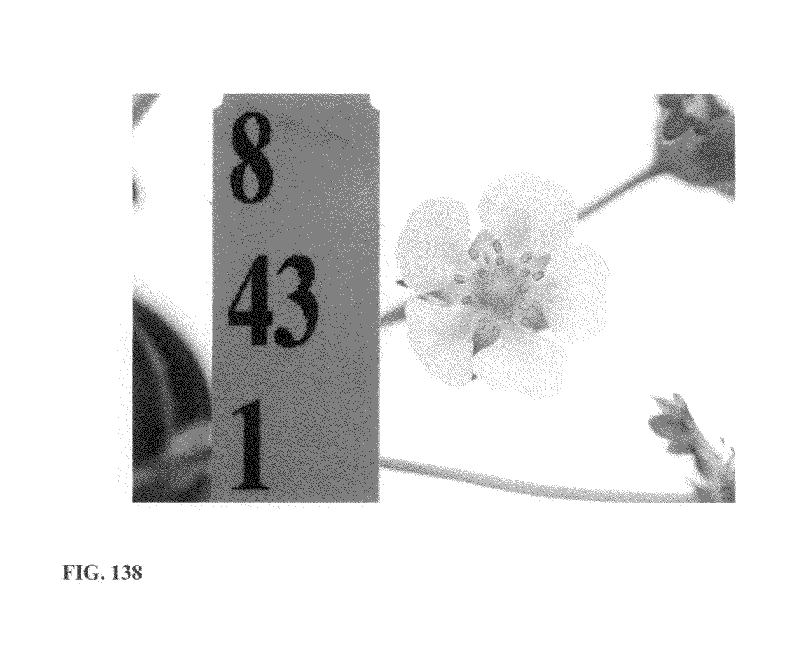

Cultivar ‘08.43-1’ has distinct disease/pathogen resistance, yield, appearance, size, and firmness. The plant of cultivar ‘08.43-1’ is characterized and distinguished in FIG. 136, the fruit of cultivar ‘08.43-1’ is characterized and distinguished in FIG. 137, and the flower of cultivar ‘08.43-1’ is characterized and distinguished in FIG. 138.

The following detailed description of cultivar ‘08.43-1’ is based on observations taken of plants and fruits (see FIGS. 136 and 137).

Certain characteristics of cultivar ‘08.43-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 46 provides data that characterizes the disease/pathogen resistance, yield, appearance, size, and firmness of cultivar ‘08.43-1’.

TABLE 46

| Cultivar | Vert | Phy | Fus | Mac | Yield | App | Size | Firm |
|---|---|---|---|---|---|---|---|---|
| 08.43-1 | 4.7 | 3.0 | 4.8 | 4.8 | 2603 | 3.8 | 33.6 | 12.5 |

### Cultivar ‘08.43-4’

In certain aspects, the present invention relates to a new and distinctive short-day type strawberry cultivar designated as ‘08.43-4’. Cultivar ‘08.43-4’ is typical of short-day strawberry cultivars and produces fruit over an extended period when treated appropriately in arid, subtropical climates.

The strawberry cultivar ‘08.43-4’ originated from a cross between two unreleased germplasm accessions ‘04.41-6’ and ‘05.99-6’. The cultivar has been asexually reproduced by runners. The properties of this cultivar were found to be transmissible by such asexual reproduction. The cultivar is stable and reproduces true to type in successive generations of asexual reproduction.

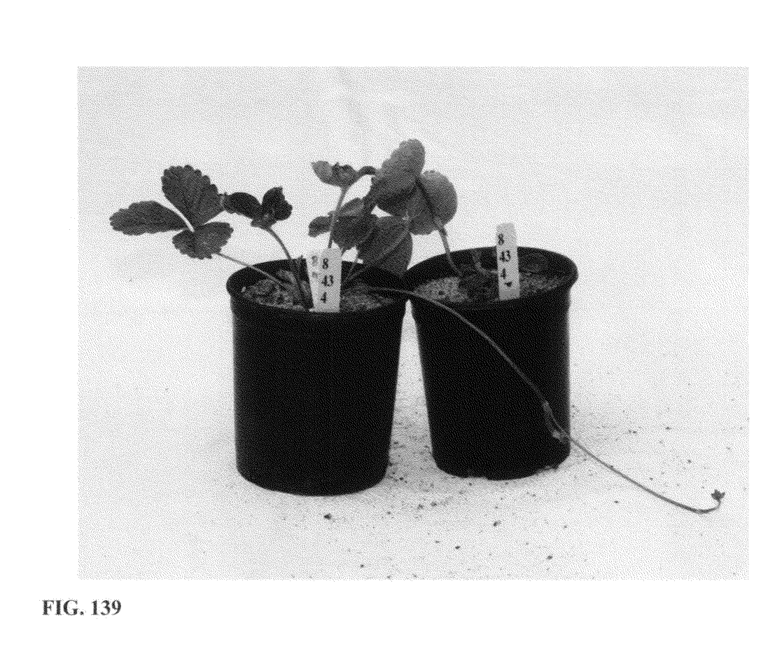

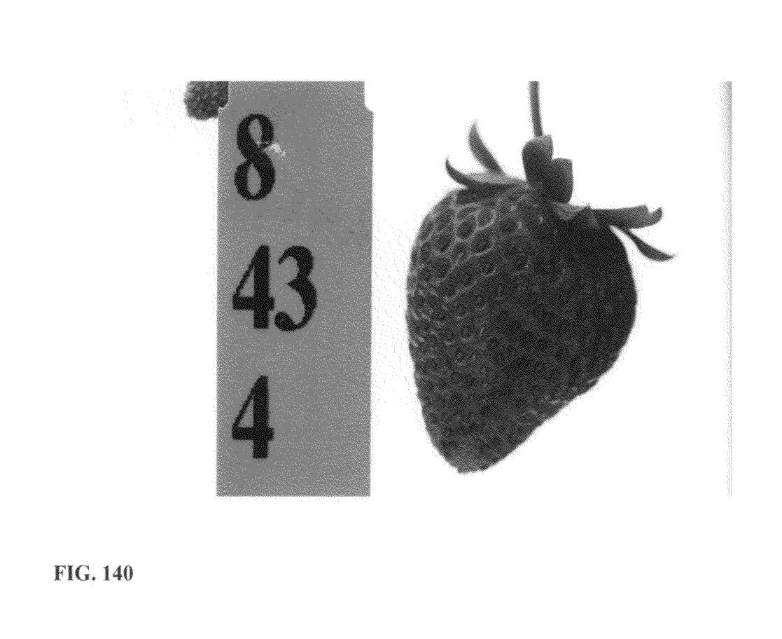

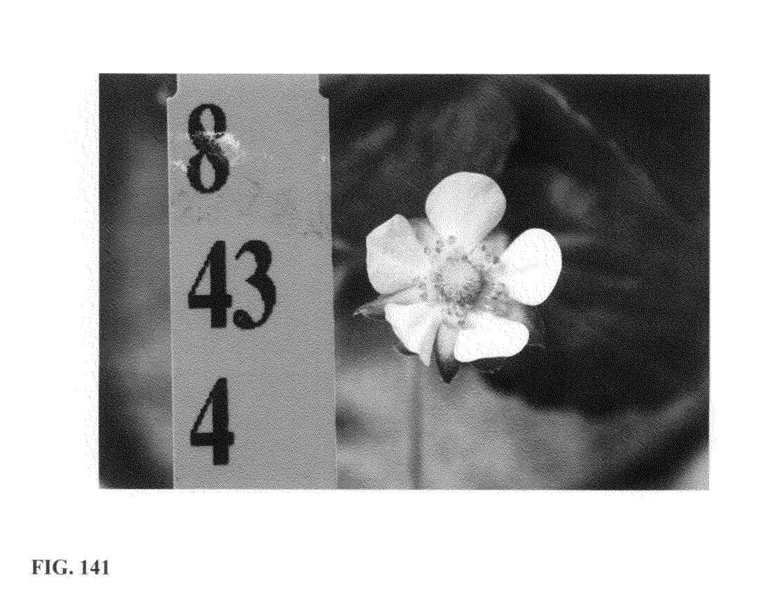

Cultivar ‘08.43-4’ has distinct disease/pathogen resistance, yield, appearance, size, and firmness. The plant of cultivar ‘08.43-4’ is characterized and distinguished in FIG. 139, the fruit of cultivar ‘08.43-4’ is characterized and distinguished in FIG. 140, and the flower of cultivar ‘08.43-4’ is characterized and distinguished in FIG. 141.

The following detailed description of cultivar ‘08.43-4’ is based on observations taken of plants and fruits (see FIGS. 139 and 140).

Certain characteristics of cultivar ‘08.43-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 47 provides data that characterizes the disease/pathogen resistance, yield, appearance, size, and firmness of cultivar ‘08.43-4’.

TABLE 47

| Cultivar | Vert | Phy | Fus | Mac | Yield | App | Size | Firm |
|---|---|---|---|---|---|---|---|---|
| 08.43-4 | 2.8 | 4.2 | 4.8 | 4.0 | 2101 | 3.7 | 36.3 | 11.1 |

### Cultivar ‘08.75-1’

In certain aspects, the present invention relates to a new and distinctive short-day type strawberry cultivar designated as ‘08.75-1’. Cultivar ‘08.75-1’ is typical of short-day strawberry cultivars and produces fruit over an extended period when treated appropriately in arid, subtropical climates.

The strawberry cultivar ‘08.75-1’ originated from a cross between two unreleased germplasm accessions ‘05.165-1’ and ‘04.53-1’. The cultivar has been asexually reproduced by runners. The properties of this cultivar were found to be transmissible by such asexual reproduction. The cultivar is stable and reproduces true to type in successive generations of asexual reproduction.

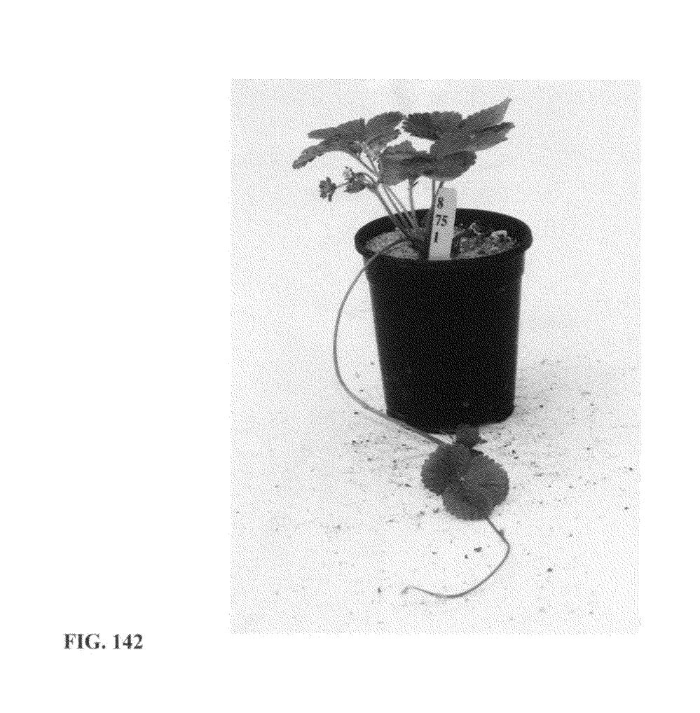

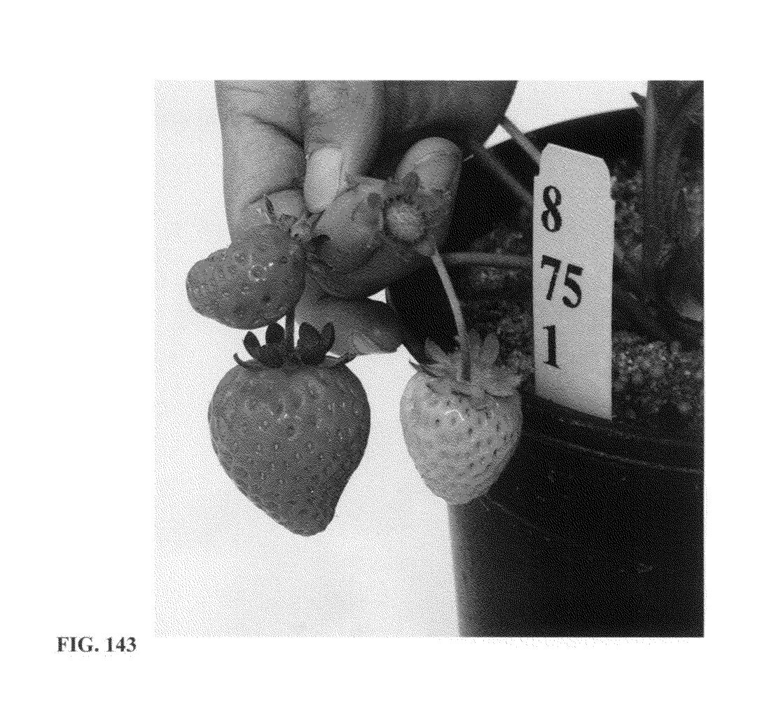

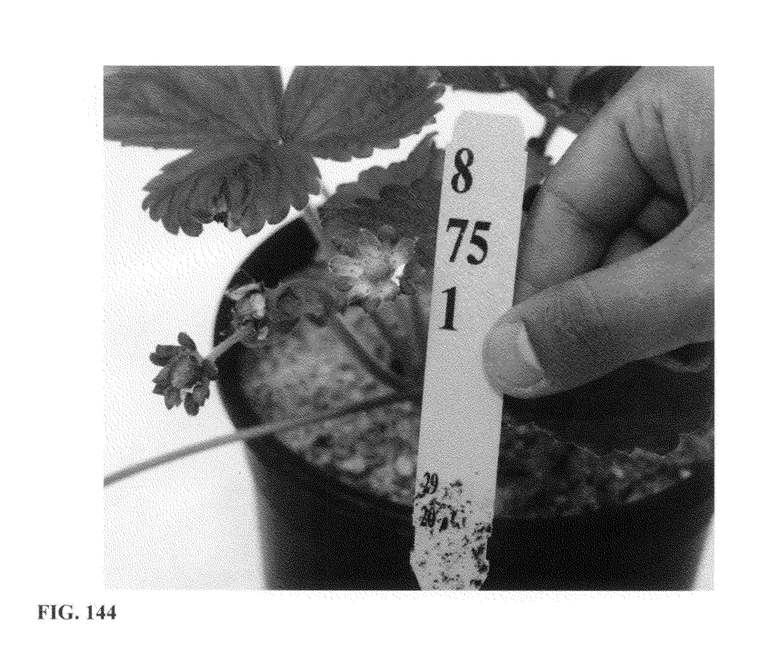

Cultivar ‘08.75-1’ has distinct disease/pathogen resistance, yield, appearance, size, and firmness. The plant of cultivar ‘08.75-1’ is characterized and distinguished in FIG. 142, the fruit of cultivar ‘08.75-1’ is characterized and distinguished in FIG. 143, and the flower of cultivar ‘08.75-1’ is characterized and distinguished in FIG. 144.

The following detailed description of cultivar ‘08.75-1’ is based on observations taken of plants and fruits (see FIGS. 142 and 143).

Certain characteristics of cultivar ‘08.75-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 48 provides data that characterizes the disease/pathogen resistance, yield, appearance, size, and firmness of cultivar ‘08.75-1’.

TABLE 48

| Cultivar | Vert | Phy | Yield | App | Size | Firm |
|---|---|---|---|---|---|---|
| 08.75-1 | 3.0 | 4.8 | 2080 | 4.0 | 36.6 | 13.7 |

### Cultivar ‘08.114-5’

In certain aspects, the present invention relates to a new and distinctive day-neutral type strawberry cultivar designated as ‘08.114-5’. Cultivar ‘08.114-5’ is typical of day-neutral strawberry cultivars and produces fruit regardless of day length when treated appropriately in arid, subtropical climates.

The strawberry cultivar ‘08.114-5’ originated from a cross between two unreleased germplasm accessions ‘05.92-14’ and ‘05.109-2’. The cultivar has been asexually reproduced by runners. The properties of this cultivar were found to be transmissible by such asexual reproduction. The cultivar is stable and reproduces true to type in successive generations of asexual reproduction.

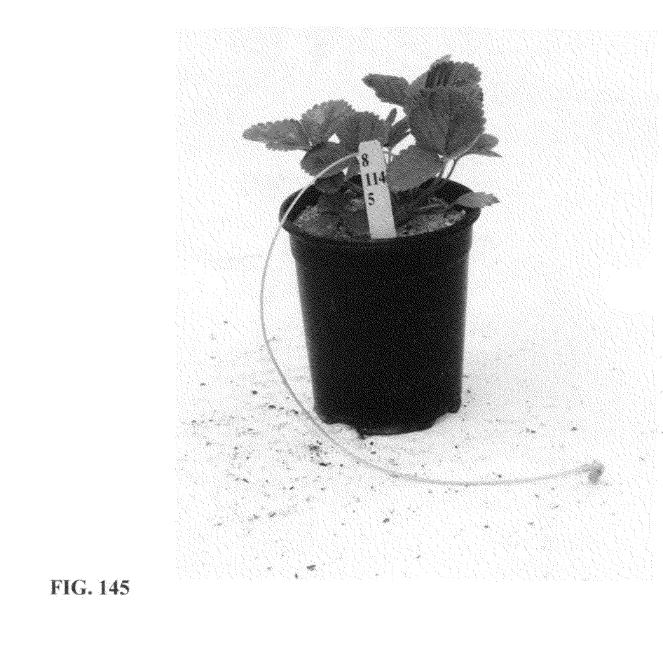

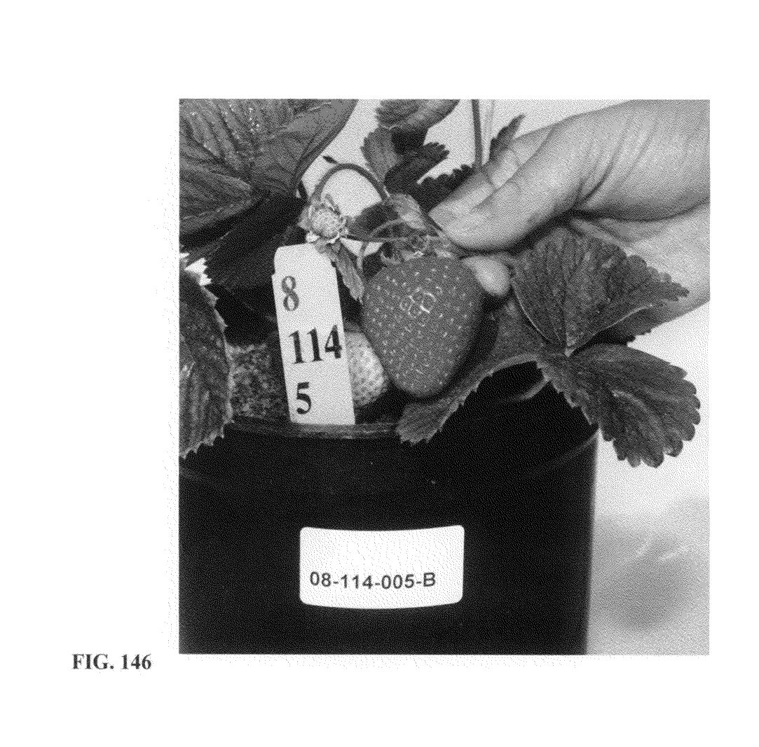

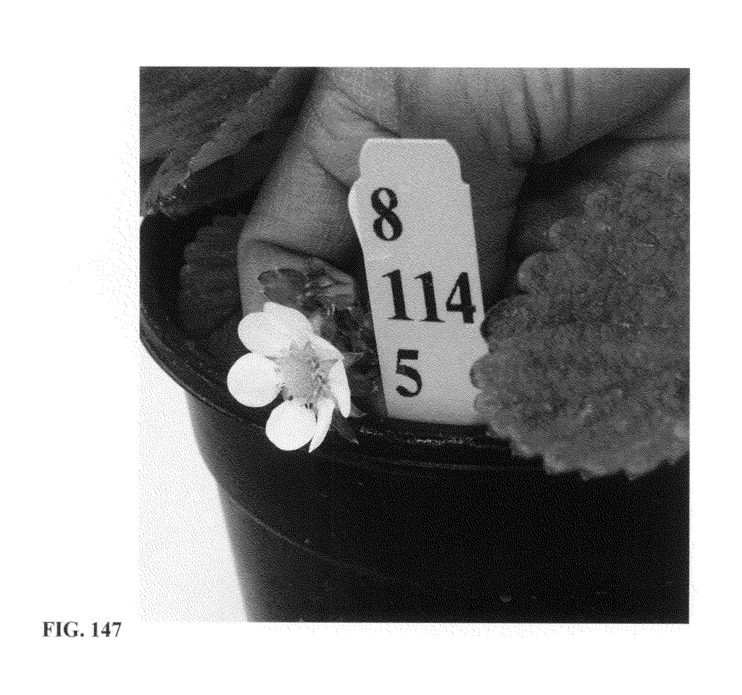

Cultivar ‘08.114-5’ has distinct disease/pathogen resistance, yield, appearance, size, and firmness. The plant of cultivar ‘08.114-5’ is characterized and distinguished in FIG. 145, the fruit of cultivar ‘08.114-5’ is characterized and distinguished in FIG. 146, and the flower of cultivar ‘08.114-5’ is characterized and distinguished in FIG. 147.

The following detailed description of cultivar ‘08.114-5’ is based on observations taken of plants and fruits (see FIGS. 145 and 146).

Certain characteristics of cultivar ‘08.114-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 49 provides data that characterizes the disease/pathogen resistance, yield, appearance, size, and firmness of cultivar ‘08.114-5’.

TABLE 49

| Cultivar | Vert | Phy | Yield | App | Size | Firm |
|---|---|---|---|---|---|---|
| 08.114-5 | 3.9 | 3.6 | 2136 | 3.6 | 29.1 | 11.2 |

### Cultivar ‘08.123-1’

In certain aspects, the present invention relates to a new and distinctive day-neutral type strawberry cultivar designated as ‘08.123-1’. Cultivar ‘08.123-1’ is typical of day-neutral strawberry cultivars and produces fruit regardless of day length when treated appropriately in arid, subtropical climates.

The strawberry cultivar ‘08.123-1’ originated from a cross between two unreleased germplasm accessions ‘03.114-3’ and ‘04.9-5’. The cultivar has been asexually reproduced by runners. The properties of this cultivar were found to be transmissible by such asexual reproduction. The cultivar is stable and reproduces true to type in successive generations of asexual reproduction.

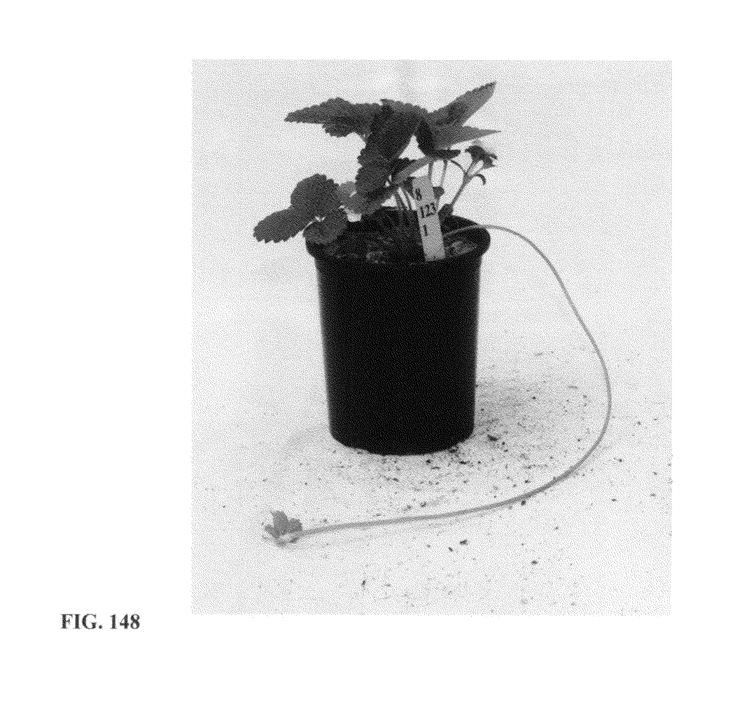

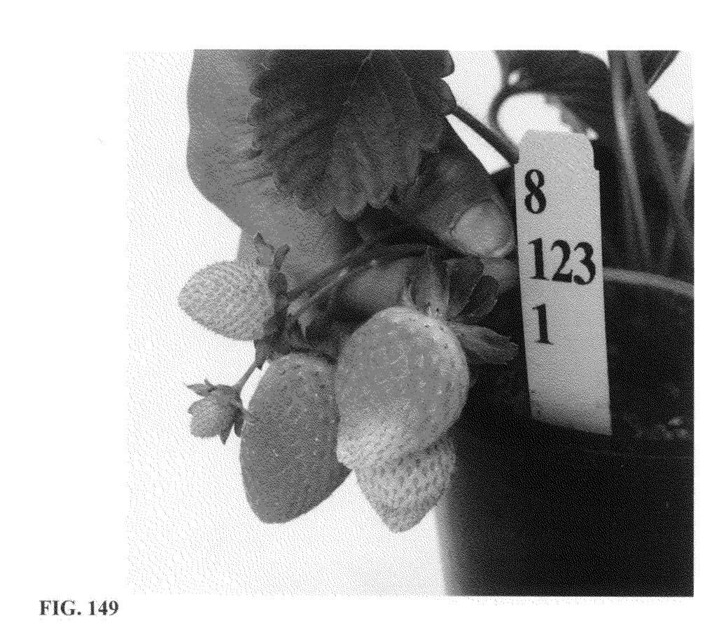

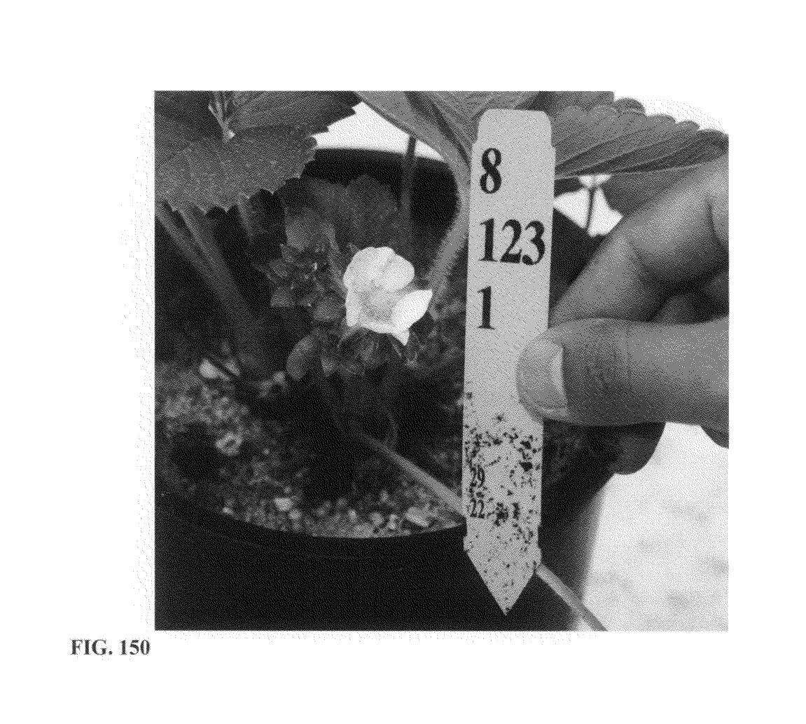

Cultivar ‘08.123-1’ has distinct disease/pathogen resistance, yield, appearance, size, and firmness. The plant of cultivar ‘08.123-1’ is characterized and distinguished in FIG. 148, the fruit of cultivar ‘08.123-1’ is characterized and distinguished in FIG. 149, and the flower of cultivar ‘08.123-1’ is characterized and distinguished in FIG. 150.

The following detailed description of cultivar ‘08.123-1’ is based on observations taken of plants and fruits (see FIGS. 148 and 149).

Certain characteristics of cultivar ‘08.123-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 50 provides data that characterizes the disease/pathogen resistance, yield, appearance, size, and firmness of cultivar ‘08.123-1’.

TABLE 50

| Cultivar | Vert | Phy | Fus | Mac | Yield | App | Size | Firm |
|---|---|---|---|---|---|---|---|---|
| 08.123-1 | 3.4 | 4.1 | 3.5 | 3.5 | 3263 | 3.6 | 32.4 | 12.2 |

### Cultivar ‘08.124-1’

In certain aspects, the present invention relates to a new and distinctive short-day type strawberry cultivar designated as ‘08.124-1’. Cultivar ‘08.124-1’ is typical of short-day strawberry cultivars and produces fruit over an extended period when treated appropriately in arid, subtropical climates.

The strawberry cultivar ‘08.124-1’ originated from a cross between two unreleased germplasm accessions ‘05.99-6’ and ‘04.9-5’. The cultivar has been asexually reproduced by runners. The properties of this cultivar were found to be transmissible by such asexual reproduction. The cultivar is stable and reproduces true to type in successive generations of asexual reproduction.

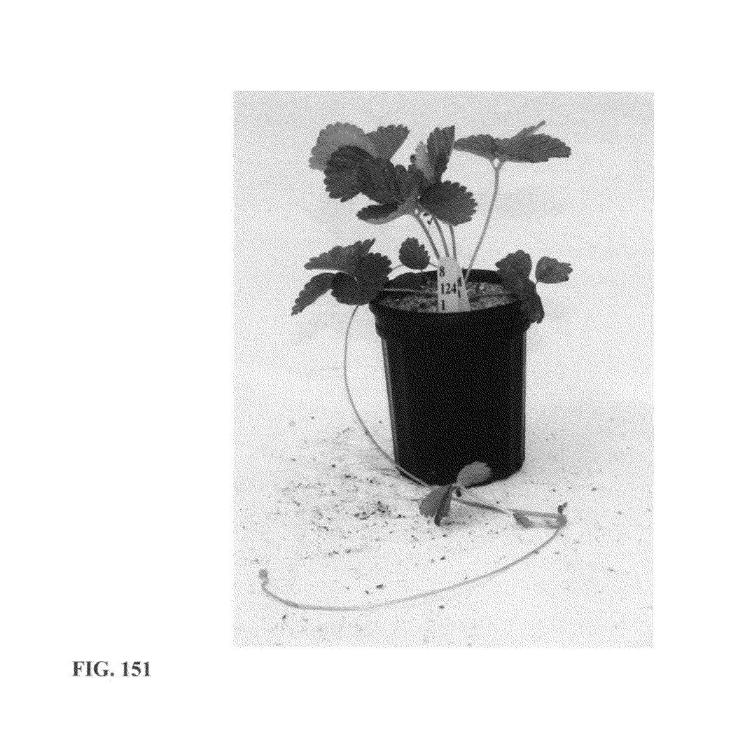

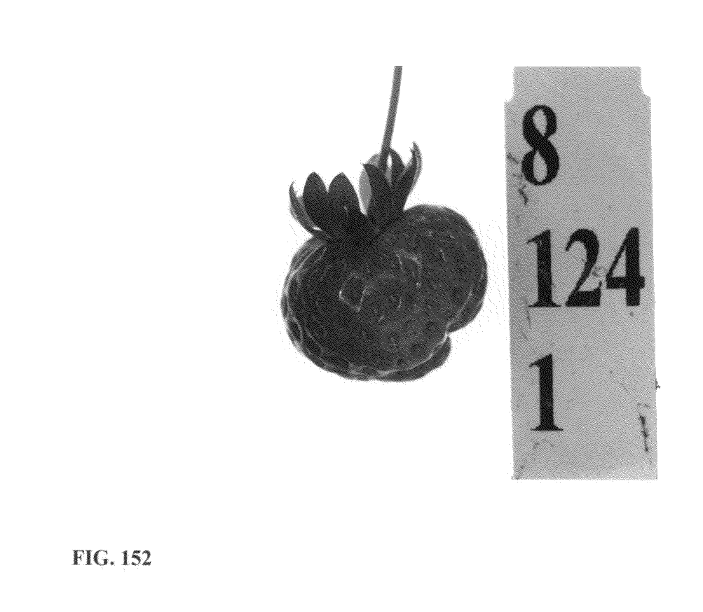

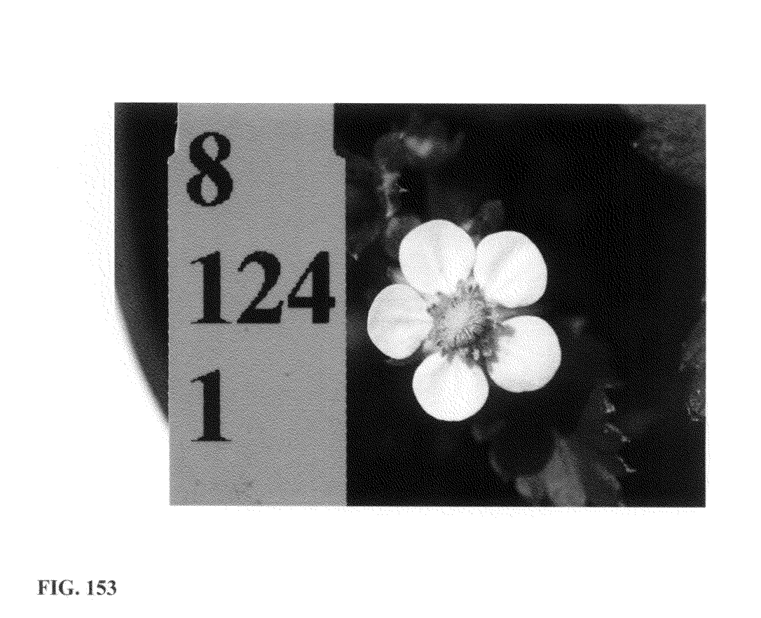

Cultivar ‘08.124-1’ has distinct disease/pathogen resistance, yield, appearance, size, and firmness. The plant of cultivar ‘08.124-1’ is characterized and distinguished in FIG. 151, the fruit of cultivar ‘08.124-1’ is characterized and distinguished in FIG. 152, and the flower of cultivar ‘08.124-1’ is characterized and distinguished in FIG. 153.

The following detailed description of cultivar ‘08.124-1’ is based on observations taken of plants and fruits (see FIGS. 151 and 152).

Certain characteristics of cultivar ‘08.124-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 51 provides data that characterizes the disease/pathogen resistance, yield, appearance, size, and firmness of cultivar ‘08.124-1’.

TABLE 51

| Cultivar | Vert | Phy | Fus | Mac | Yield | App | Size | Firm |
|---|---|---|---|---|---|---|---|---|
| 08.124-1 | 4.0 | 4.6 | 5.0 | 4.5 | 2774 | 3.9 | 34.0 | 11.9 |

### Cultivar ‘08.128-2’

In certain aspects, the present invention relates to a new and distinctive day-neutral type strawberry cultivar designated as ‘08.128-2’. Cultivar ‘08.128-2’ is typical of day-neutral strawberry cultivars and produces fruit regardless of day length when treated appropriately in arid, subtropical climates.

The strawberry cultivar ‘08.128-2’ originated from a cross between two unreleased germplasm accessions ‘03.102-2’ and ‘04.18-4’. The cultivar has been asexually reproduced by runners. The properties of this cultivar were found to be transmissible by such asexual reproduction. The cultivar is stable and reproduces true to type in successive generations of asexual reproduction.

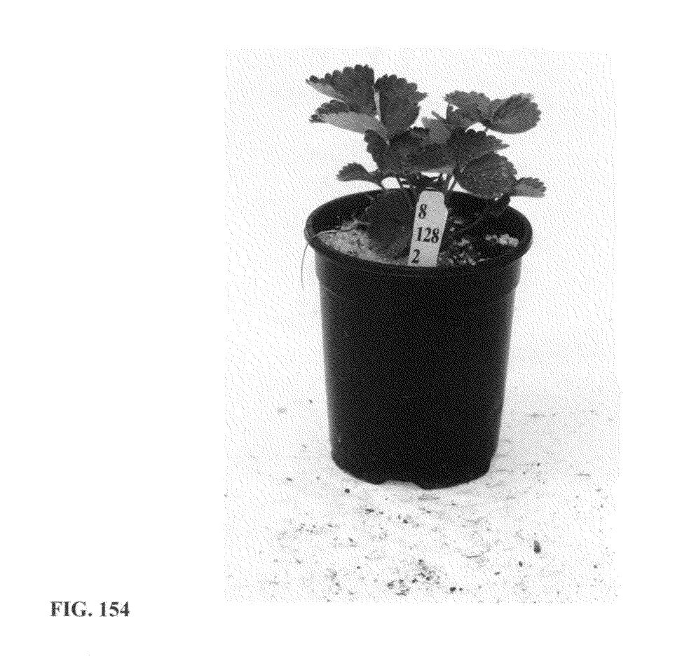

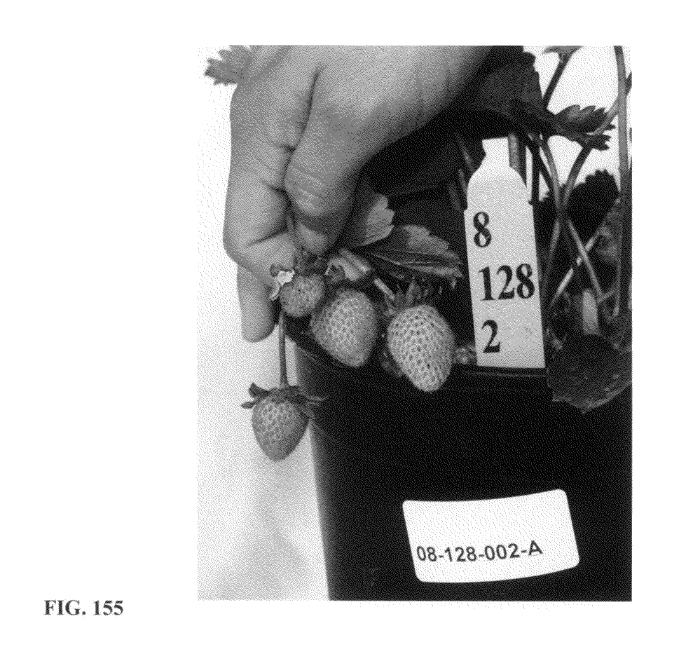

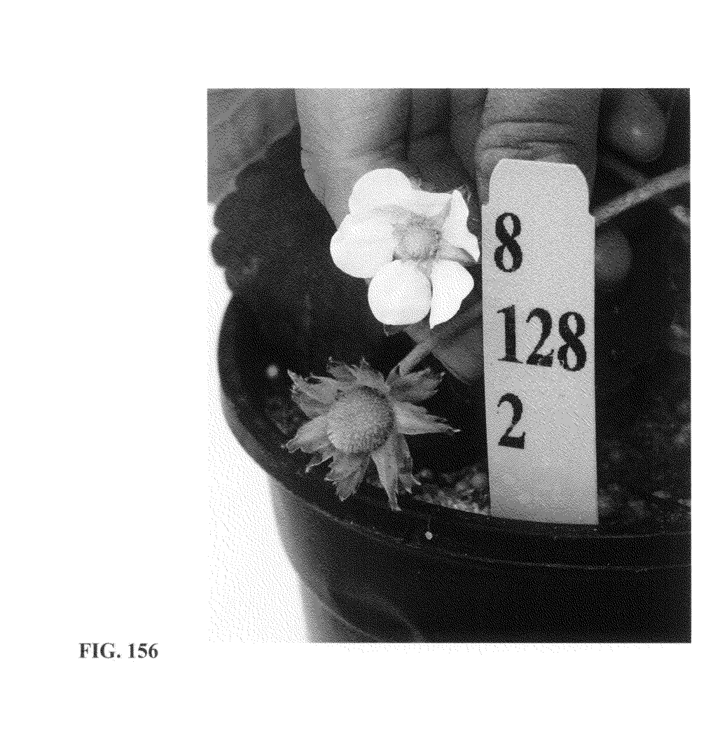

Cultivar ‘08.128-2’ has distinct disease/pathogen resistance, yield, appearance, size, and firmness. The plant of cultivar ‘08.128-2’ is characterized and distinguished in FIG. 154, the fruit of cultivar ‘08.128-2’ is characterized and distinguished in FIG. 155, and the flower of cultivar ‘08.128-2’ is characterized and distinguished in FIG. 156.

The following detailed description of cultivar ‘08.128-2’ is based on observations taken of plants and fruits (see FIGS. 154 and 155).

Certain characteristics of cultivar ‘08.128-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 52 provides data that characterizes the disease/pathogen resistance, yield, appearance, size, and firmness of cultivar ‘08.128-2’.

TABLE 52

| Cultivar | Vert | Phy | Fus | Mac | Yield | App | Size | Firm |
|---|---|---|---|---|---|---|---|---|
| 08.128-2 | 4.5 | 4.1 | 4.8 | 4.6 | 2516 | 3.5 | 34.0 | 10.4 |

### Cultivar ‘08.129-4’

In certain aspects, the present invention relates to a new and distinctive day-neutral type strawberry cultivar designated as ‘08.129-4’. Cultivar ‘08.129-4’ is typical of day-neutral strawberry cultivars and produces fruit regardless of day length when treated appropriately in arid, subtropical climates.

The strawberry cultivar ‘08.129-4’ originated from a cross between two unreleased germplasm accessions ‘03.114-3’ and ‘04.18-4’. The cultivar has been asexually reproduced by runners. The properties of this cultivar were found to be transmissible by such asexual reproduction. The cultivar is stable and reproduces true to type in successive generations of asexual reproduction.

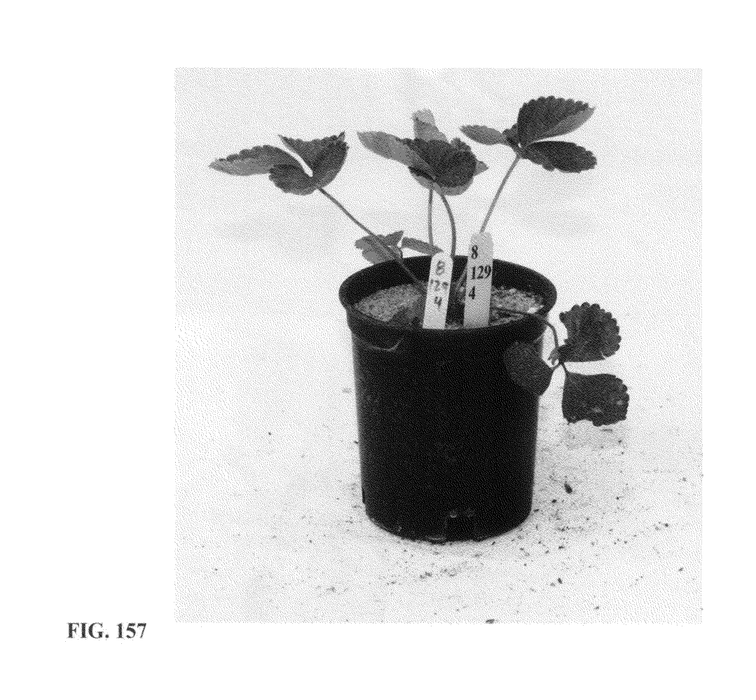

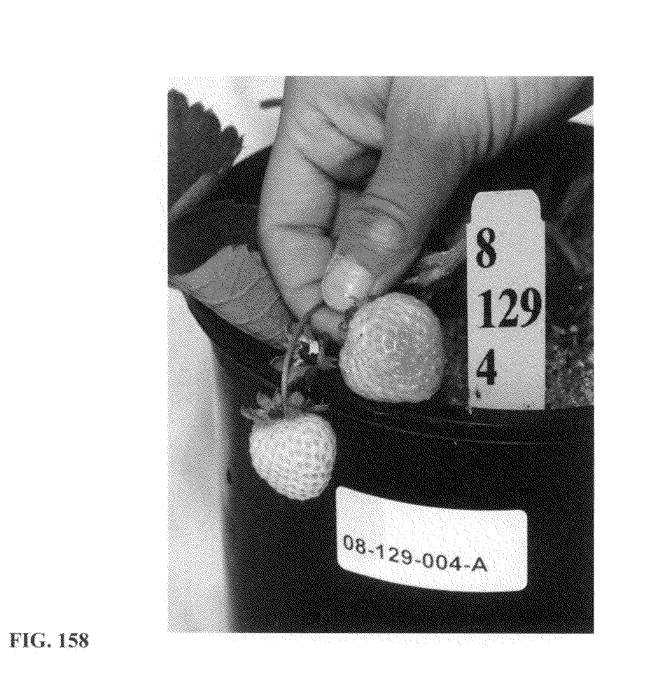

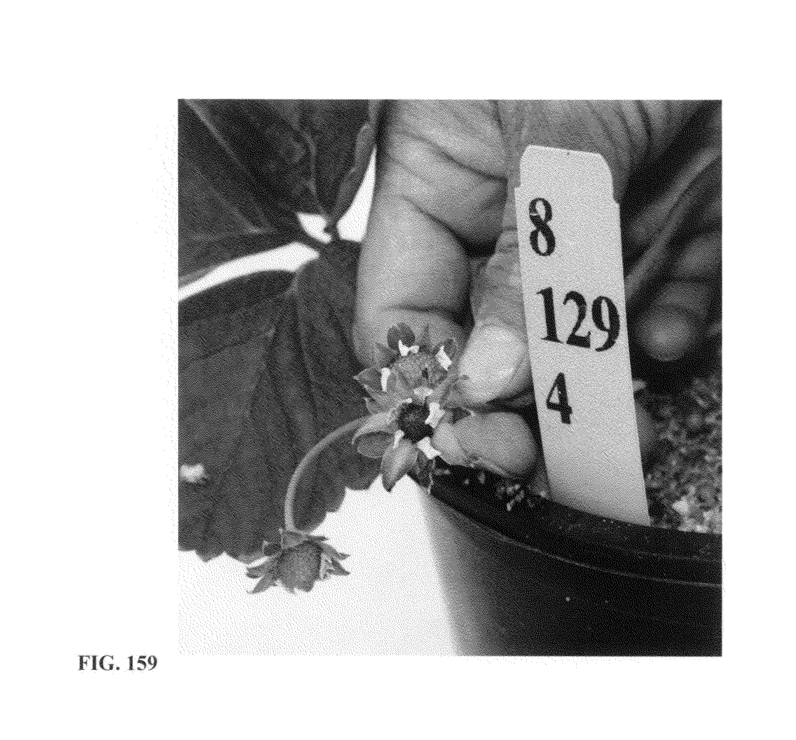

Cultivar ‘08.129-4’ has distinct disease/pathogen resistance, yield, appearance, size, and firmness. The plant of cultivar ‘08.129-4’ is characterized and distinguished in FIG. 157, the fruit of cultivar ‘08.129-4’ is characterized and distinguished in FIG. 158, and the flower of cultivar ‘08.129-4’ is characterized and distinguished in FIG. 159.

The following detailed description of cultivar ‘08.129-4’ is based on observations taken of plants and fruits (see FIGS. 157 and 158).

Certain characteristics of cultivar ‘08.129-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 53 provides data that characterizes the disease/pathogen resistance, yield, appearance, size, and firmness of cultivar ‘08.129-4’.

TABLE 53

| Cultivar | Vert | Phy | Fus | Mac | Yield | App | Size | Firm |
|---|---|---|---|---|---|---|---|---|
| 08.129-4 | 4.8 | 4.4 | 4.9 | 3.0 | 2827 | 3.8 | 32.3 | 11.2 |

### Cultivar ‘08.131-3’

In certain aspects, the present invention relates to a new and distinctive short-day type strawberry cultivar designated as ‘08.131-3’. Cultivar ‘08.131-3’ is typical of short-day strawberry cultivars and produces fruit over an extended period when treated appropriately in arid, subtropical climates.

The strawberry cultivar ‘08.131-3’ originated from a cross between two unreleased germplasm accessions ‘05.109-2’ and ‘04.18-4’. The cultivar has been asexually reproduced by runners. The properties of this cultivar were found to be transmissible by such asexual reproduction. The cultivar is stable and reproduces true to type in successive generations of asexual reproduction.

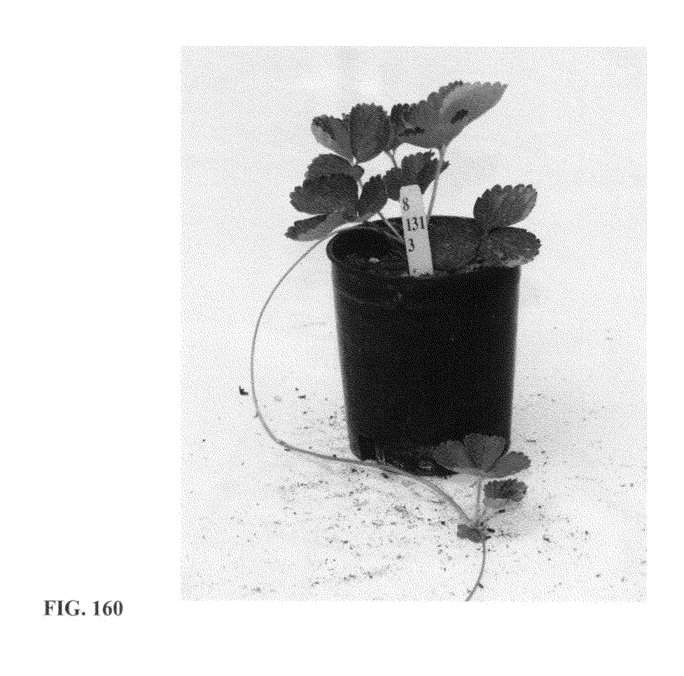

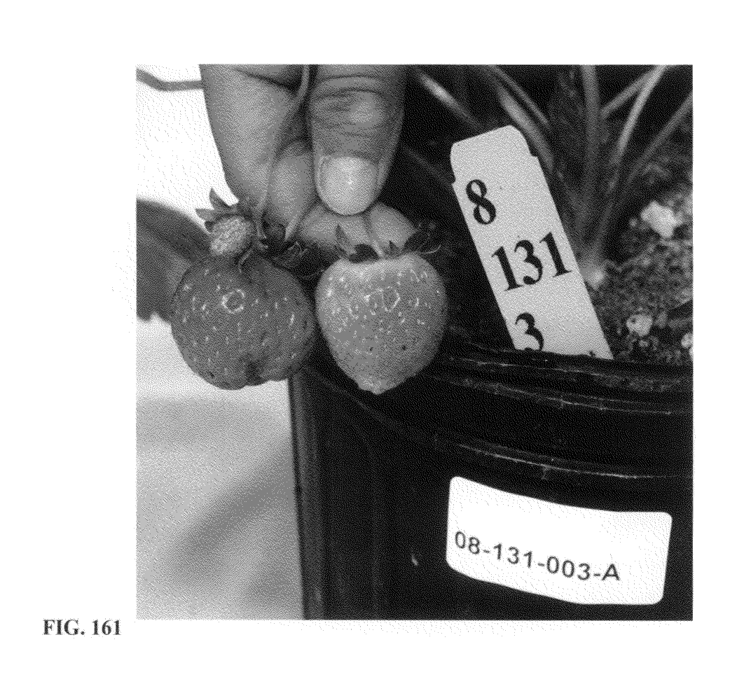

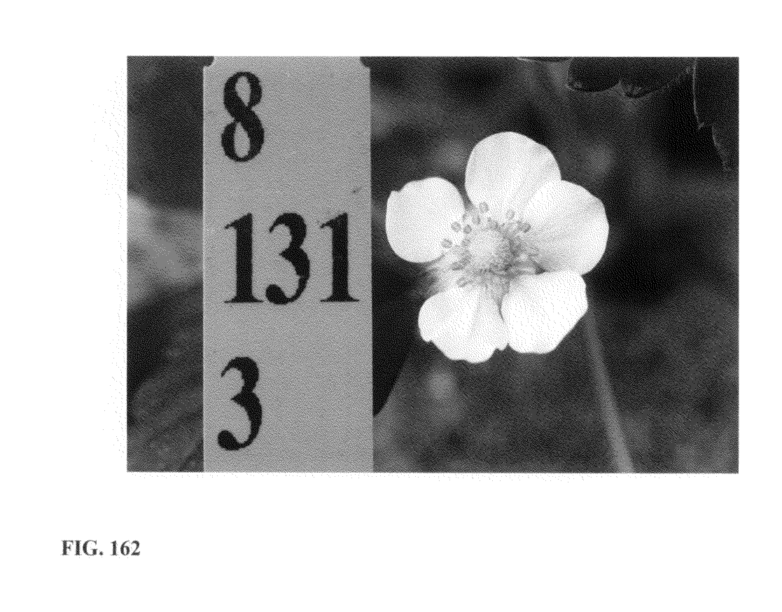

Cultivar ‘08.131-3’ has distinct disease/pathogen resistance, yield, appearance, size, and firmness. The plant of cultivar ‘08.131-3’ is characterized and distinguished in FIG. 160, the fruit of cultivar ‘08.131-3’ is characterized and distinguished in FIG. 161, and the flower of cultivar ‘08.131-3’ is characterized and distinguished in FIG. 162.

The following detailed description of cultivar ‘08.131-3’ is based on observations taken of plants and fruits (see FIGS. 160 and 161).

Certain characteristics of cultivar ‘08.131-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 54 provides data that characterizes the disease/pathogen resistance, yield, appearance, size, and firmness of cultivar ‘08.131-3’.

TABLE 54

| Cultivar | Vert | Phy | Fus | Mac | Yield | App | Size | Firm |
|---|---|---|---|---|---|---|---|---|
| 08.131-3 | 4.5 | 4.5 | 4.9 | 4.8 | 2701 | 3.7 | 34.3 | 10.3 |

### Cultivar ‘08.132-3’

In certain aspects, the present invention relates to a new and distinctive short-day type strawberry cultivar designated as ‘08.132-3’. Cultivar ‘08.132-3’ is typical of short-day strawberry cultivars and produces fruit over an extended period when treated appropriately in arid, subtropical climates.

The strawberry cultivar ‘08.132-3’ originated from a cross between two unreleased germplasm accessions ‘05.165-1’ and ‘04.18-4’. The cultivar has been asexually reproduced by runners. The properties of this cultivar were found to be transmissible by such asexual reproduction. The cultivar is stable and reproduces true to type in successive generations of asexual reproduction.

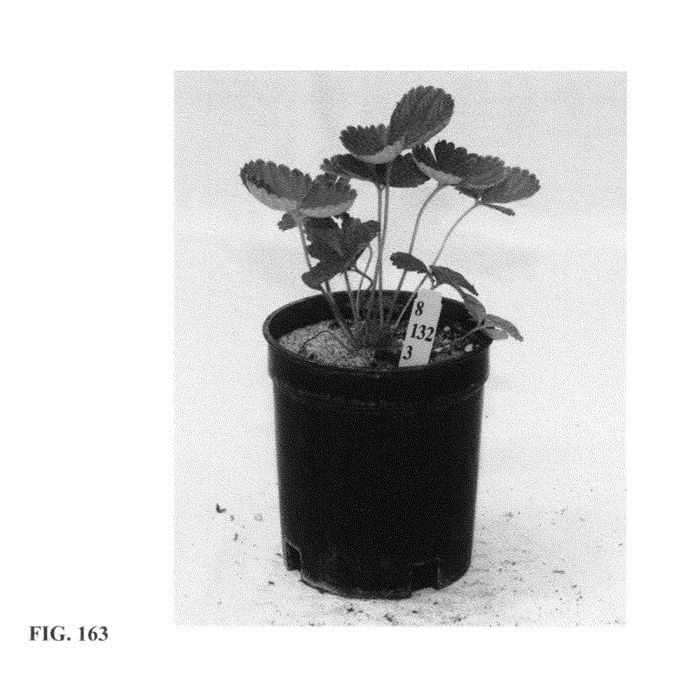

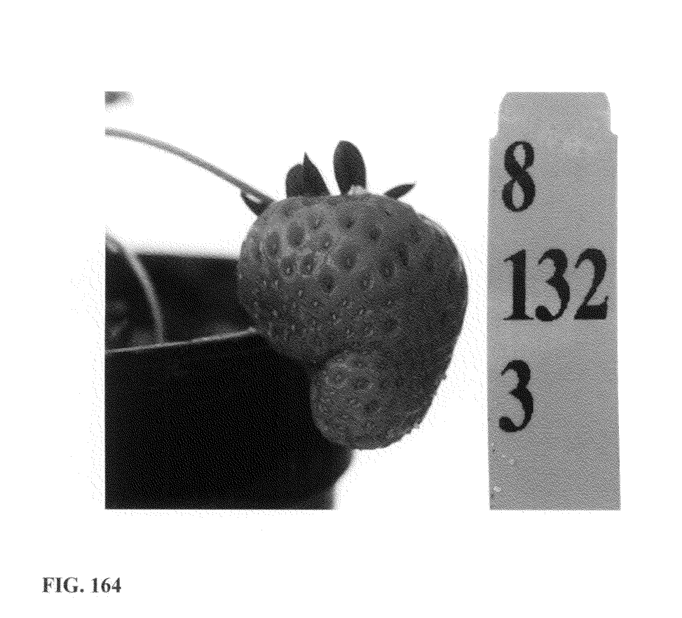

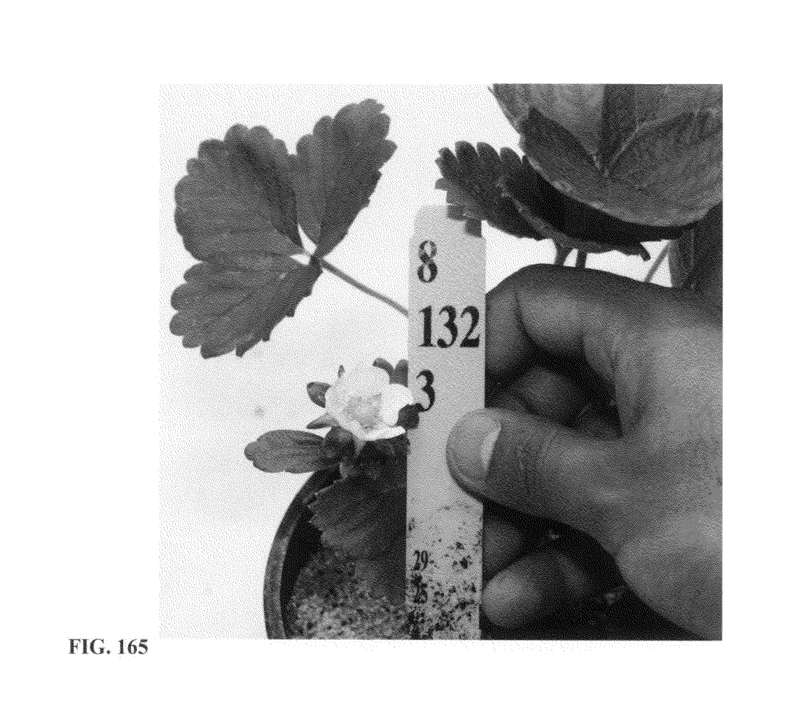

Cultivar ‘08.132-3’ has distinct disease/pathogen resistance, yield, appearance, size, and firmness. The plant of cultivar ‘08.132-3’ is characterized and distinguished in FIG. 163, the fruit of cultivar ‘08.132-3’ is characterized and distinguished in FIG. 164, and the flower of cultivar ‘08.132-3’ is characterized and distinguished in FIG. 165.

The following detailed description of cultivar ‘08.132-3’ is based on observations taken of plants and fruits (see FIGS. 163 and 164).

Certain characteristics of cultivar ‘08.132-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 55 provides data that characterizes the disease/pathogen resistance, yield, appearance, size, and firmness of cultivar ‘08.132-3’.

TABLE 55

| Cultivar | Vert | Phy | Yield | App | Size | Firm |
|---|---|---|---|---|---|---|
| 08.132-3 | 5.0 | 3.6 | 2540 | 3.9 | 37.4 | 12.2 |

### Cultivar ‘08.137-1’

In certain aspects, the present invention relates to a new and distinctive short-day type strawberry cultivar designated as ‘08.137-1’. Cultivar ‘08.137-1’ is typical of short-day strawberry cultivars and produces fruit over an extended period when treated appropriately in arid, subtropical climates.

The strawberry cultivar ‘08.137-1’ originated from a cross between two unreleased germplasm accessions ‘04.41-6’ and ‘04.1-601’. The cultivar has been asexually reproduced by runners. The properties of this cultivar were found to be transmissible by such asexual reproduction. The cultivar is stable and reproduces true to type in successive generations of asexual reproduction.

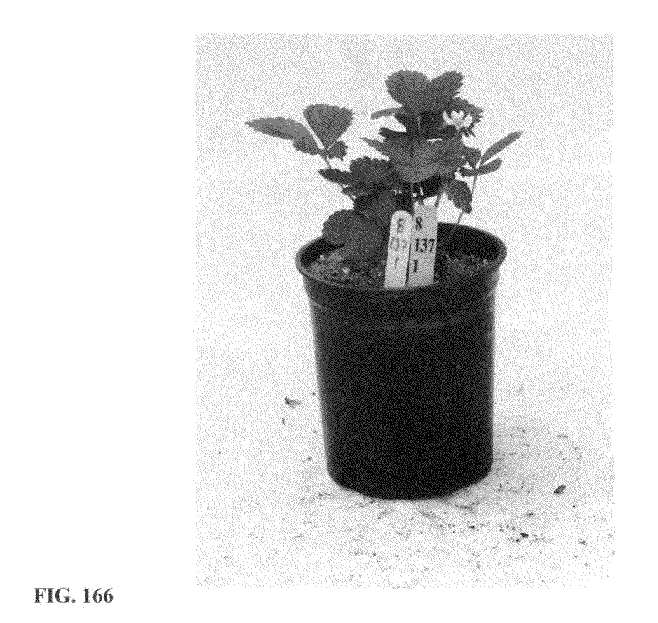

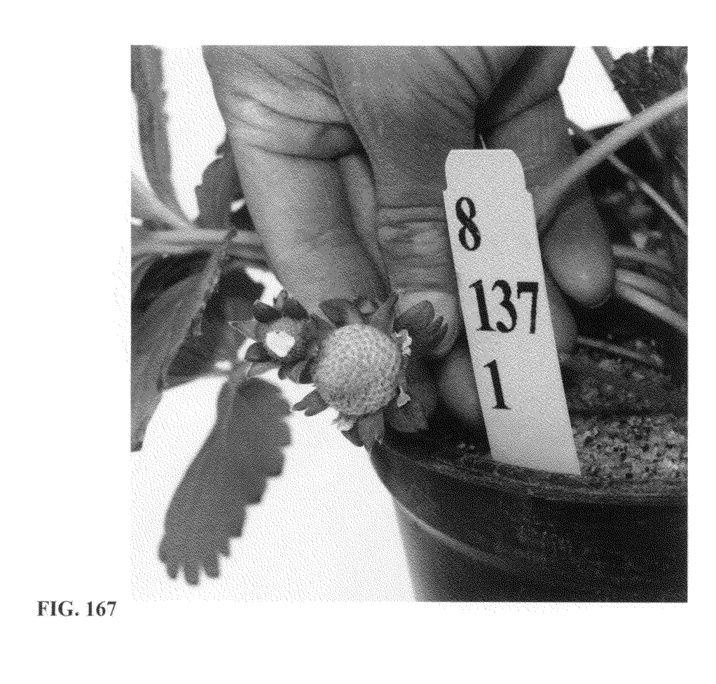

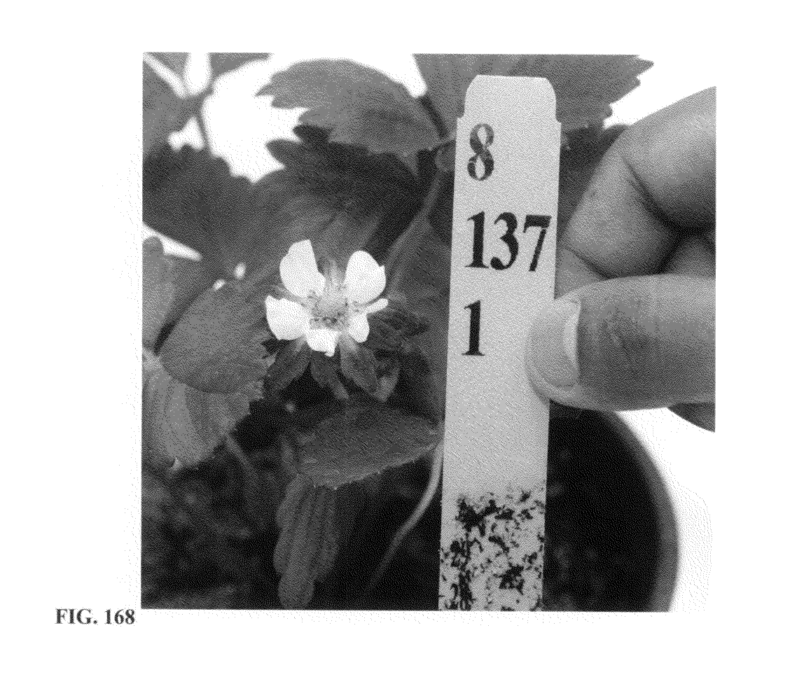

Cultivar ‘08.137-1’ has distinct disease/pathogen resistance, yield, appearance, size, and firmness. The plant of cultivar ‘08.137-1’ is characterized and distinguished in FIG. 166, the fruit of cultivar ‘08.137-1’ is characterized and distinguished in FIG. 167, and the flower of cultivar ‘08.137-1’ is characterized and distinguished in FIG. 168.

The following detailed description of cultivar ‘08.137-1’ is based on observations taken of plants and fruits (see FIGS. 166 and 167).

Certain characteristics of cultivar ‘08.137-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 56 provides data that characterizes the disease/pathogen resistance, yield, appearance, size, and firmness of cultivar ‘08.137-1’.

TABLE 56

| Cultivar | Vert | Phy | Fus | Yield | App | Size | Firm |
|---|---|---|---|---|---|---|---|
| 08.137-1 | 3.4 | N/A | 4.6 | 2524 | 3.6 | 35.7 | 12.5 |

### Cultivar ‘08.138-3’

In certain aspects, the present invention relates to a new and distinctive short-day type strawberry cultivar designated as ‘08.138-3’. Cultivar ‘08.138-3’ is typical of short-day strawberry cultivars and produces fruit over an extended period when treated appropriately in arid, subtropical climates.

The strawberry cultivar ‘08.138-3’ originated from a cross between two unreleased germplasm accessions ‘05.165-1’ and ‘04.76-4’. The cultivar has been asexually reproduced by runners. The properties of this cultivar were found to be transmissible by such asexual reproduction. The cultivar is stable and reproduces true to type in successive generations of asexual reproduction.

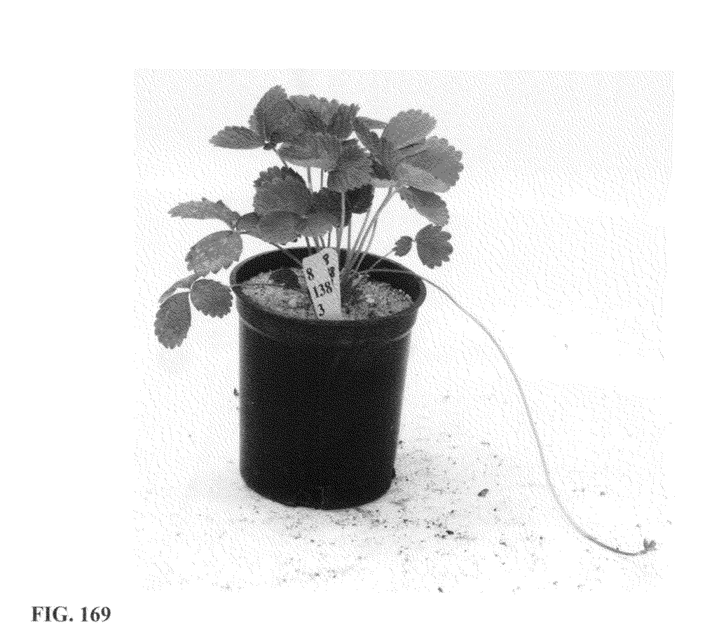

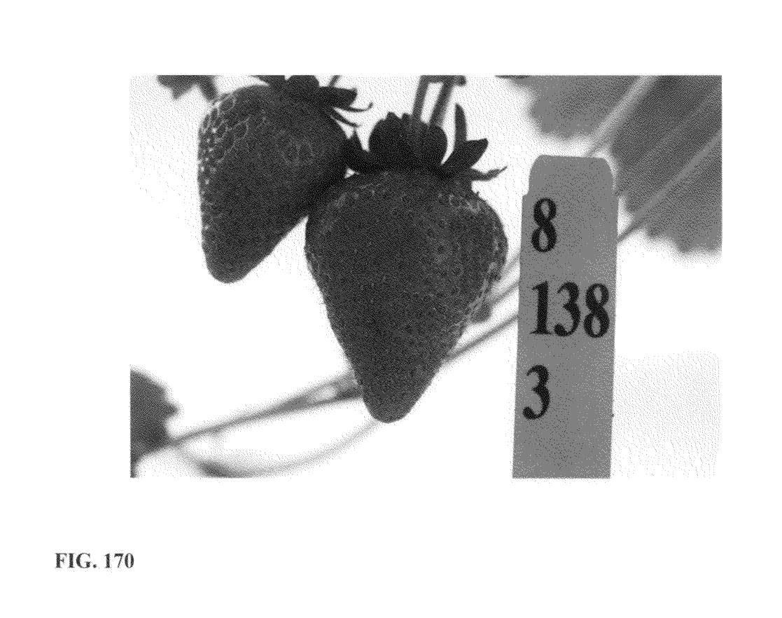

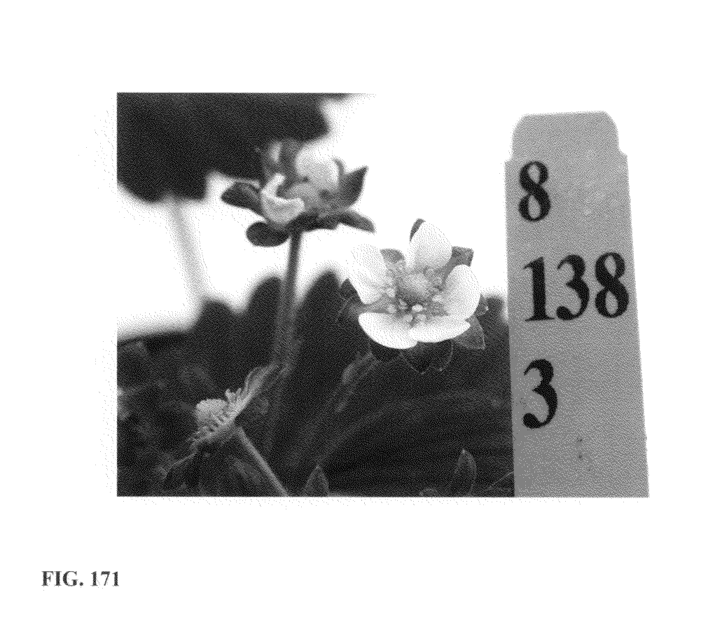

Cultivar ‘08.138-3’ has distinct disease/pathogen resistance, yield, appearance, size, and firmness. The plant of cultivar ‘08.138-3’ is characterized and distinguished in FIG. 169, the fruit of cultivar ‘08.138-3’ is characterized and distinguished in FIG. 170, and the flower of cultivar ‘08.138-3’ is characterized and distinguished in FIG. 171.

The following detailed description of cultivar ‘08.138-3’ is based on observations taken of plants and fruits (see FIGS. 169 and 170).

Certain characteristics of cultivar ‘08.138-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 57 provides data that characterizes the disease/pathogen resistance, yield, appearance, size, and firmness of cultivar ‘08.138-3’.

TABLE 57

| Cultivar | Vert | Phy | Fus | Mac | Yield | App | Size | Firm |
|---|---|---|---|---|---|---|---|---|
| 08.138-3 | 2.8 | 4.1 | 4.9 | 4.8 | 2691 | 3.4 | 32.8 | 10.2 |

### Cultivar ‘08.150-9’

In certain aspects, the present invention relates to a new and distinctive day-neutral type strawberry cultivar designated as ‘08.150-9’. Cultivar ‘08.150-9’ is typical of day-neutral strawberry cultivars and produces fruit regardless of day length when treated appropriately in arid, subtropical climates.

The strawberry cultivar ‘08.150-9’ originated from a cross between two unreleased germplasm accessions ‘05.165-1’ and ‘05.199-10’. The cultivar has been asexually reproduced by runners. The properties of this cultivar were found to be transmissible by such asexual reproduction. The cultivar is stable and reproduces true to type in successive generations of asexual reproduction.

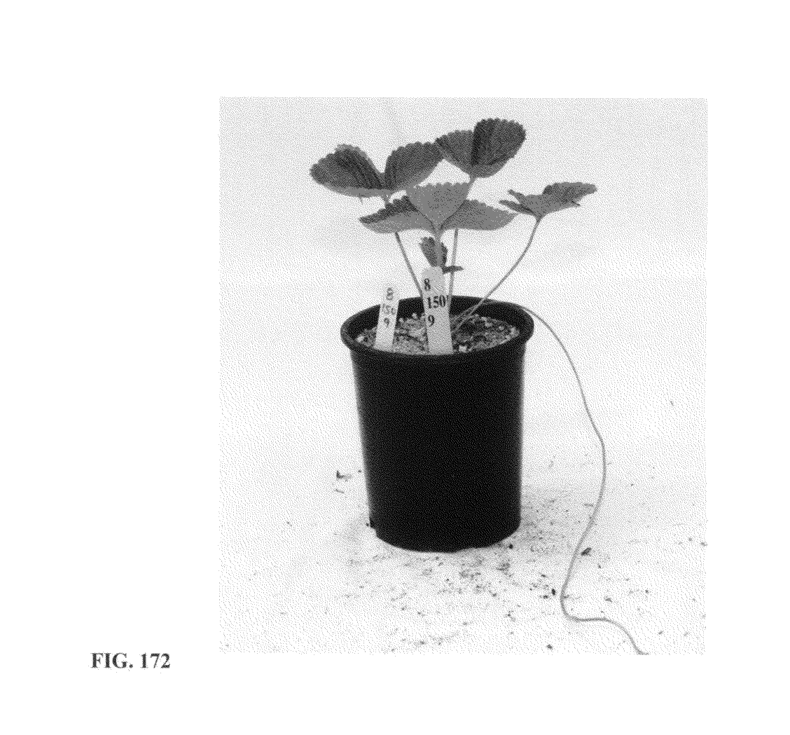

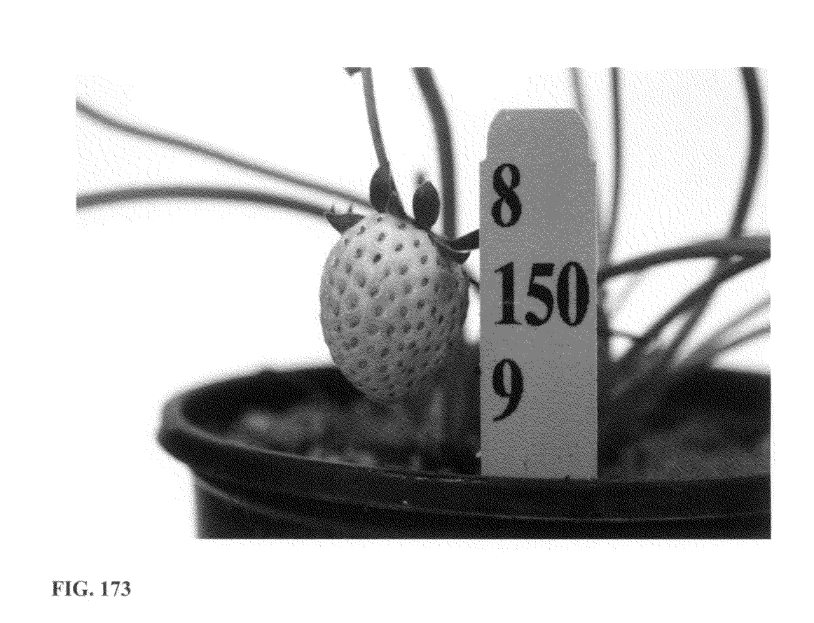

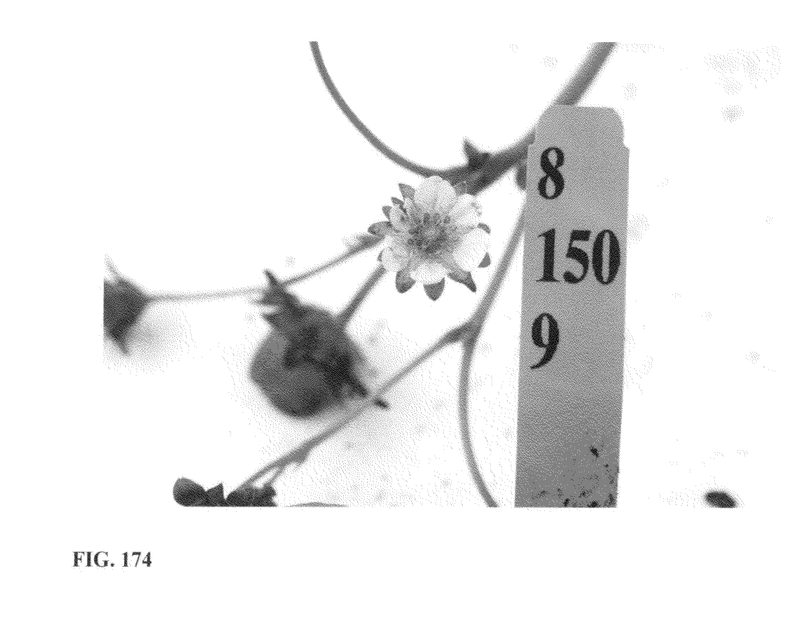

Cultivar ‘08.150-9’ has distinct disease/pathogen resistance, yield, appearance, size, and firmness. The plant of cultivar ‘08.150-9’ is characterized and distinguished in FIG. 172, the fruit of cultivar ‘08.150-9’ is characterized and distinguished in FIG. 173, and the flower of cultivar ‘08.150-9’ is characterized and distinguished in FIG. 174.

The following detailed description of cultivar ‘08.150-9’ is based on observations taken of plants and fruits (see FIGS. 172 and 173).

Certain characteristics of cultivar ‘08.150-9’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 58 provides data that characterizes the disease/pathogen resistance, yield, appearance, size, and firmness of cultivar ‘08.150-9’.

TABLE 58

| Cultivar | Vert | Phy | Fus | Yield | App | Size | Firm |
|---|---|---|---|---|---|---|---|
| 08.150-9 | 4.3 | 4.3 | 2.3 | 2467 | 4.1 | 35.9 | 11.8 |

### Cultivar ‘09.12-605’

In certain aspects, the present invention relates to a new and distinctive short-day type strawberry cultivar designated as ‘09.12-605’. Cultivar ‘09.12-605’ is typical of short-day strawberry cultivars and produces fruit over an extended period when treated appropriately in arid, subtropical climates.

The strawberry cultivar ‘09.12-605’ originated from a cross between cultivar ‘Mojave’ (U.S. Plant Pat. No. 22,589) and unreleased germplasm accession ‘05.165-1’. The cultivar has been asexually reproduced by runners. The properties of this cultivar were found to be transmissible by such asexual reproduction. The cultivar is stable and reproduces true to type in successive generations of asexual reproduction.

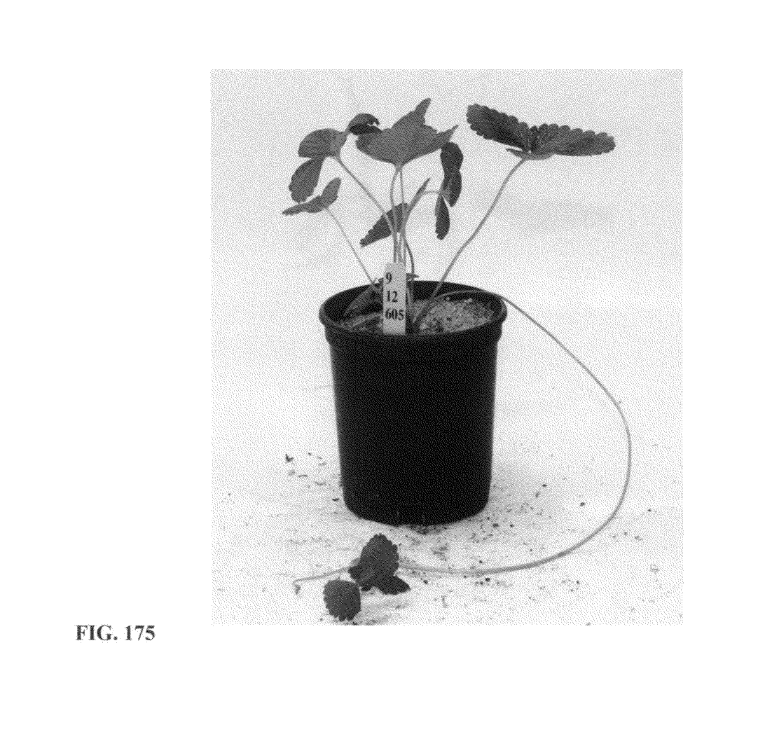

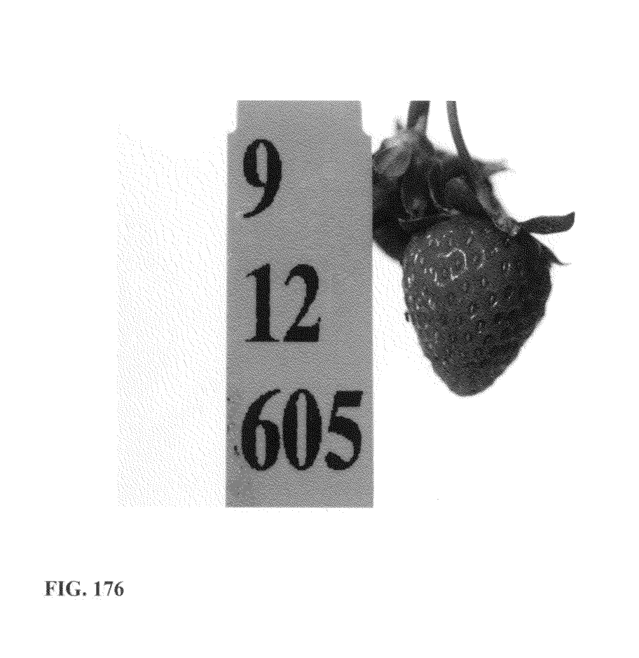

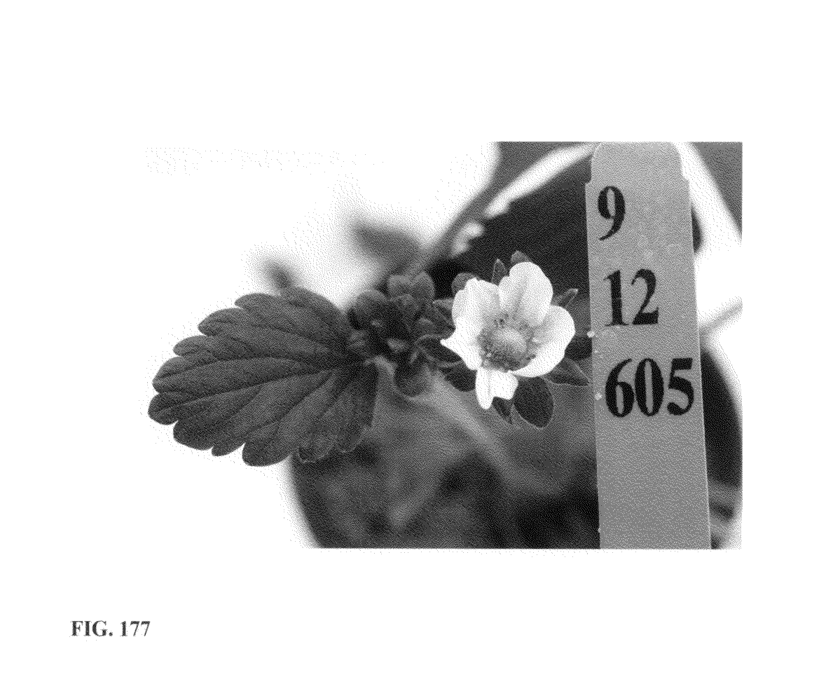

Cultivar ‘09.12-605’ has distinct disease/pathogen resistance, yield, appearance, size, and firmness. The plant of cultivar ‘09.12-605’ is characterized and distinguished in FIG. 175, the fruit of cultivar ‘09.12-605’ is characterized and distinguished in FIG. 176, and the flower of cultivar ‘09.12-605’ is characterized and distinguished in FIG. 177.

The following detailed description of cultivar ‘09.12-605’ is based on observations taken of plants and fruits (see FIGS. 175 and 176).

Certain characteristics of cultivar ‘09.12-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 59 provides data that characterizes the disease/pathogen resistance, yield, appearance, size, and firmness of cultivar ‘09.12-605’.

TABLE 59

| Cultivar | Vert | Phy | Fus | Yield | App | Size | Firm |
|---|---|---|---|---|---|---|---|
| 09.12-605 | 2.5 | 4.2 | 1.9 | 2071 | 3.8 | 33.2 | 10.7 |

### Cultivar ‘09.16-601’

In certain aspects, the present invention relates to a new and distinctive short-day type strawberry cultivar designated as ‘09.16-601’. Cultivar ‘09.16-601’ is typical of short-day strawberry cultivars and produces fruit over an extended period when treated appropriately in arid, subtropical climates.

The strawberry cultivar ‘09.16-601’ originated from a cross between two unreleased germplasm accessions ‘03.132-3’ and ‘06.132-2’. The cultivar has been asexually reproduced by runners. The properties of this cultivar were found to be transmissible by such asexual reproduction. The cultivar is stable and reproduces true to type in successive generations of asexual reproduction.

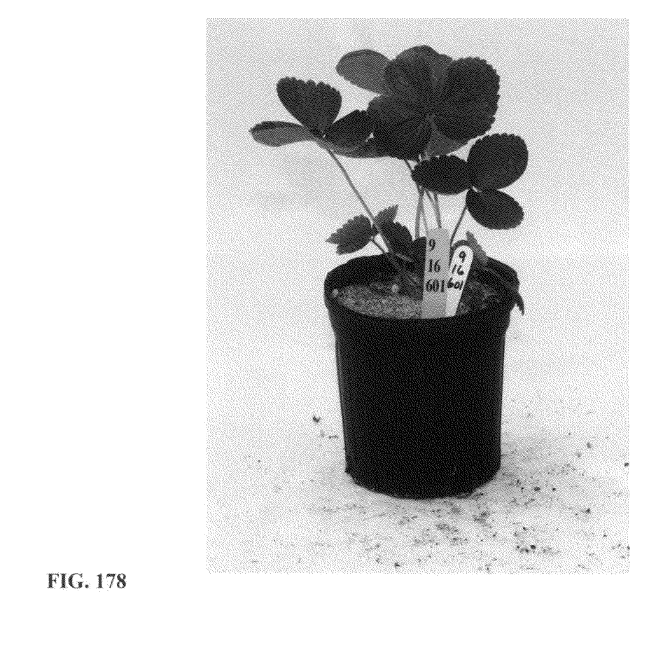

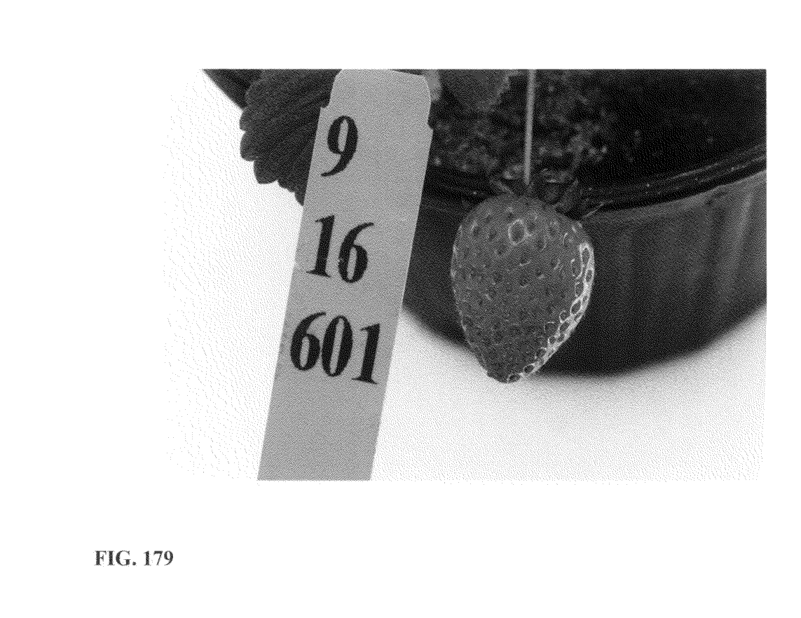

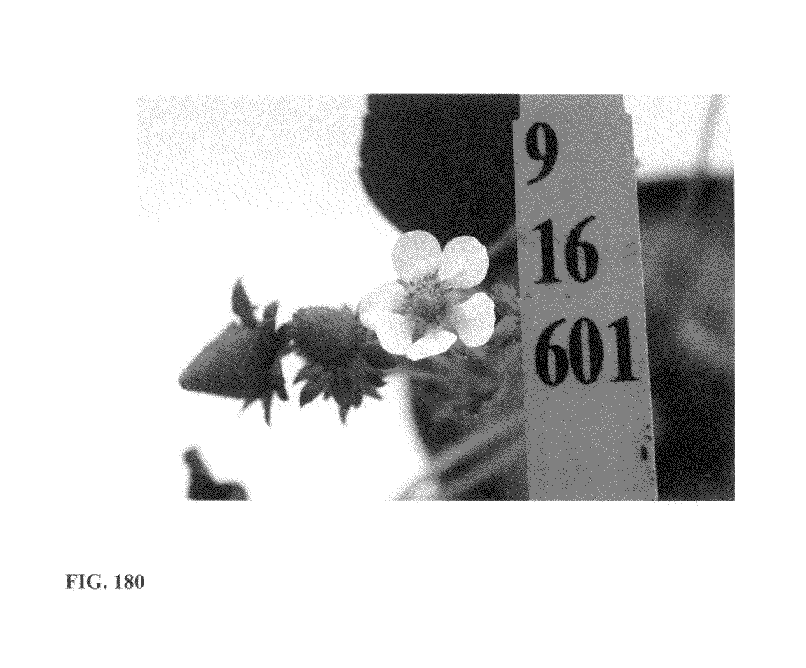

Cultivar ‘09.16-601’ has distinct disease/pathogen resistance, yield, appearance, size, and firmness. The plant of cultivar ‘09.16-601’ is characterized and distinguished in FIG. 178, the fruit of cultivar ‘09.16-601’ is characterized and distinguished in FIG. 179, and the flower of cultivar ‘09.16-601’ is characterized and distinguished in FIG. 180.

The following detailed description of cultivar ‘09.16-601’ is based on observations taken of plants and fruits (see FIGS. 178 and 179).

Certain characteristics of cultivar ‘09.16-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 60 provides data that characterizes the yield, appearance, size, and firmness of cultivar ‘09.16-601’.

TABLE 60

| Cultivar | Yield | App | Size | Firm |
|---|---|---|---|---|
| 09.16-601 | 1063 | 36.3 | 3.6 | 3.6 |

### Cultivar ‘09.20-609’

In certain aspects, the present invention relates to a new and distinctive short-day type strawberry cultivar designated as ‘09.20-609’. Cultivar ‘09.20-609’ is typical of short-day strawberry cultivars and produces fruit over an extended period when treated appropriately in arid, subtropical climates.

The strawberry cultivar ‘09.20-609’ originated from a cross between two unreleased germplasm accessions ‘05.95-4’ and ‘06.132-2’. The cultivar has been asexually reproduced by runners. The properties of this cultivar were found to be transmissible by such asexual reproduction. The cultivar is stable and reproduces true to type in successive generations of asexual reproduction.

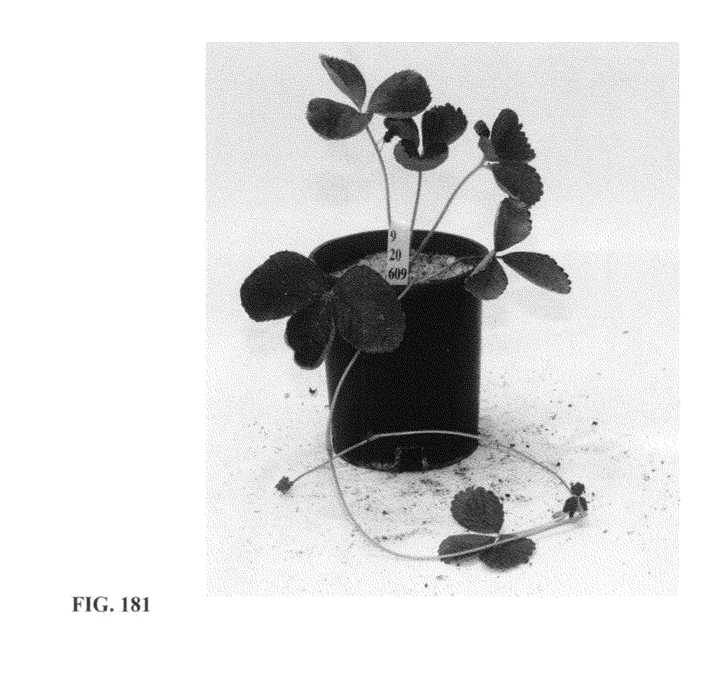

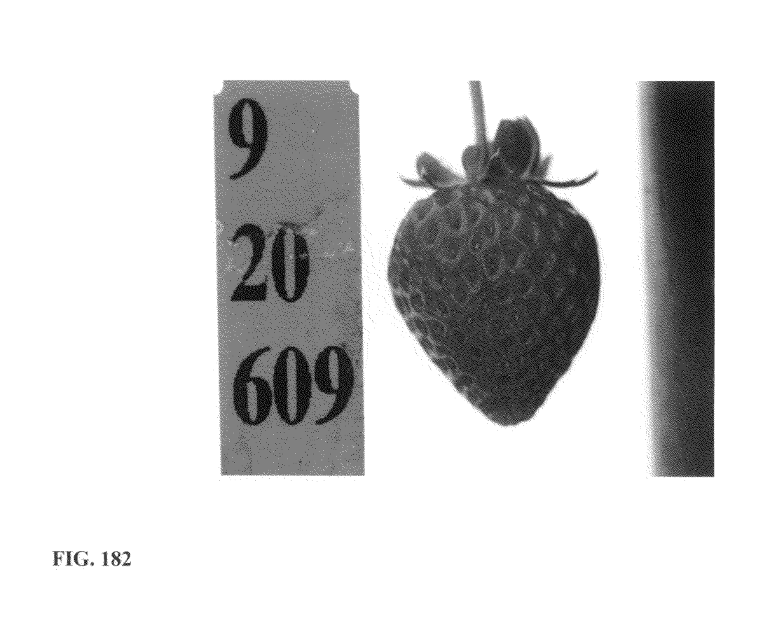

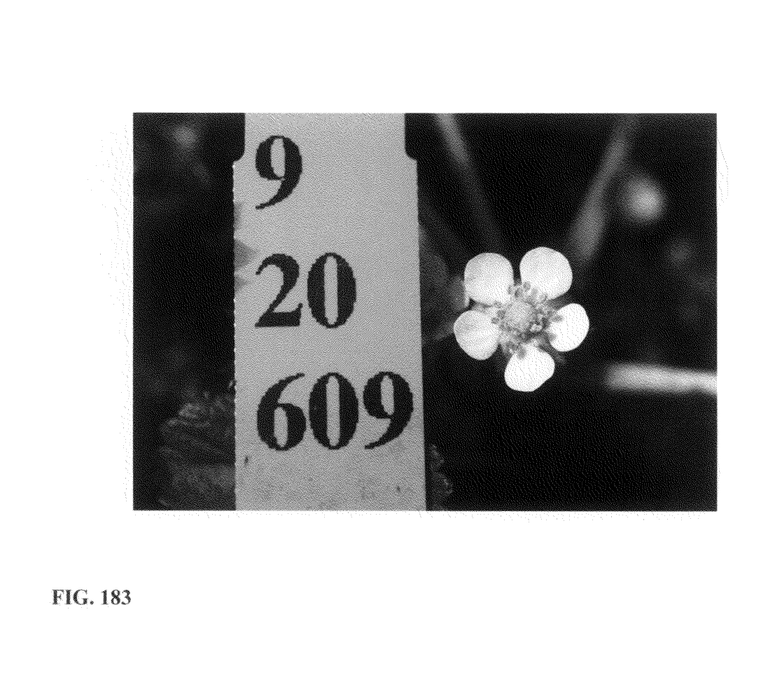

Cultivar ‘09.20-609’ has distinct disease/pathogen resistance, yield, appearance, size, and firmness. The plant of cultivar ‘09.20-609’ is characterized and distinguished in FIG. 181, the fruit of cultivar ‘09.20-609’ is characterized and distinguished in FIG. 182, and the flower of cultivar ‘09.20-609’ is characterized and distinguished in FIG. 183.

The following detailed description of cultivar ‘09.20-609’ is based on observations taken of plants and fruits (see FIGS. 181 and 182).

Certain characteristics of cultivar ‘09.20-609’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 61 provides data that characterizes the yield, appearance, size, and firmness of cultivar ‘09.20-609’.

TABLE 61

| Cultivar | Yield | App | Size | Firm |
|---|---|---|---|---|
| 09.20-609 | 2068 | 3.6 | 33.0 | 10.6 |

### Cultivar ‘09.32-605’

In certain aspects, the present invention relates to a new and distinctive short-day type strawberry cultivar designated as ‘09.32-605’. Cultivar ‘09.32-605’ is typical of short-day strawberry cultivars and produces fruit over an extended period when treated appropriately in arid, subtropical climates.

The strawberry cultivar ‘09.32-605’ originated from a cross between two unreleased germplasm accessions ‘04.45-605’ and ‘05.107-2’. The cultivar has been asexually reproduced by runners. The properties of this cultivar were found to be transmissible by such asexual reproduction. The cultivar is stable and reproduces true to type in successive generations of asexual reproduction.

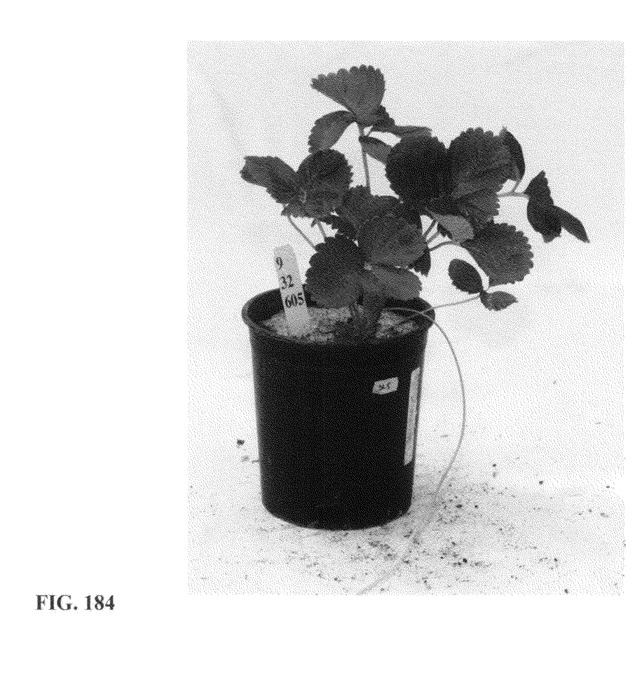

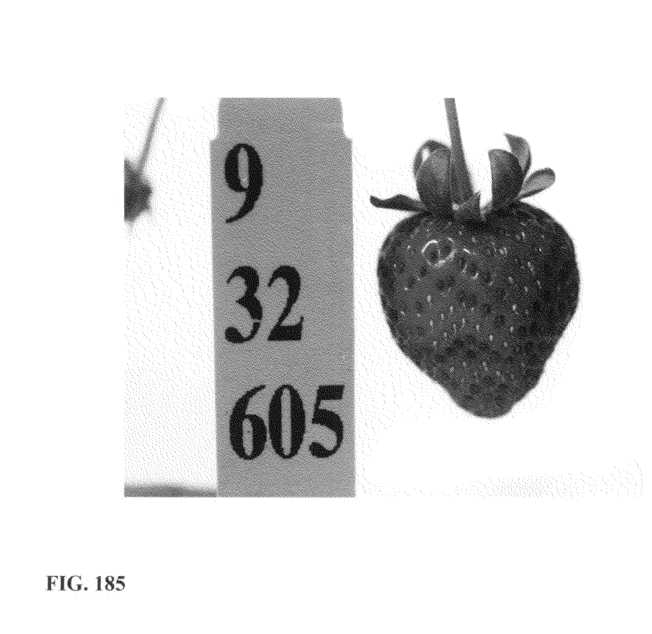

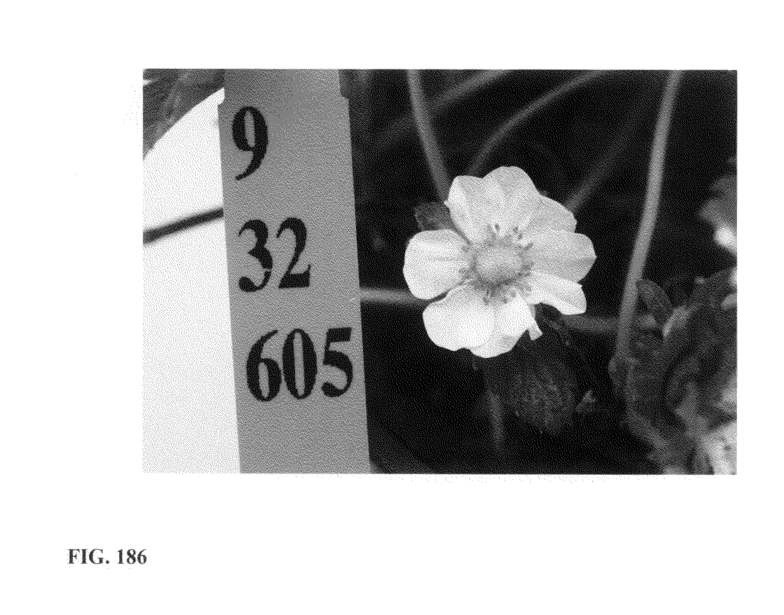

Cultivar ‘09.32-605’ has distinct disease/pathogen resistance, yield, appearance, size, and firmness. The plant of cultivar ‘09.32-605’ is characterized and distinguished in FIG. 184, the fruit of cultivar ‘09.32-605’ is characterized and distinguished in FIG. 185, and the flower of cultivar ‘09.32-605’ is characterized and distinguished in FIG. 186.

The following detailed description of cultivar ‘09.32-605’ is based on observations taken of plants and fruits (see FIGS. 184 and 185).

Certain characteristics of cultivar ‘09.32-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 62 provides data that characterizes the yield, appearance, size, and firmness of cultivar ‘09.32-605’.

TABLE 62

| Cultivar | Yield | App | Size | Firm |
|---|---|---|---|---|
| 09.32-605 | 2053 | 3.4 | 29.5 | 11.7 |

### Cultivar ‘09.37-1’

In certain aspects, the present invention relates to a new and distinctive short-day type strawberry cultivar designated as ‘09.37-1’. Cultivar ‘09.37-1’ is typical of short-day strawberry cultivars and produces fruit over an extended period when treated appropriately in arid, subtropical climates.

The strawberry cultivar ‘09.37-1’ originated from a cross between cultivar ‘Mojave’ (U.S. Plant Pat. No. 22,589) and unreleased germplasm accession ‘05.120-9’. The cultivar has been asexually reproduced by runners. The properties of this cultivar were found to be transmissible by such asexual reproduction. The cultivar is stable and reproduces true to type in successive generations of asexual reproduction.

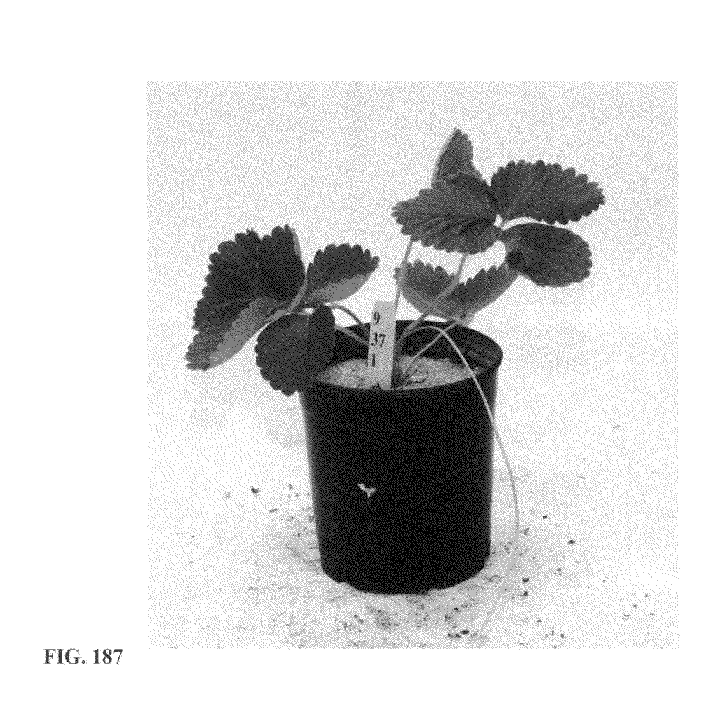

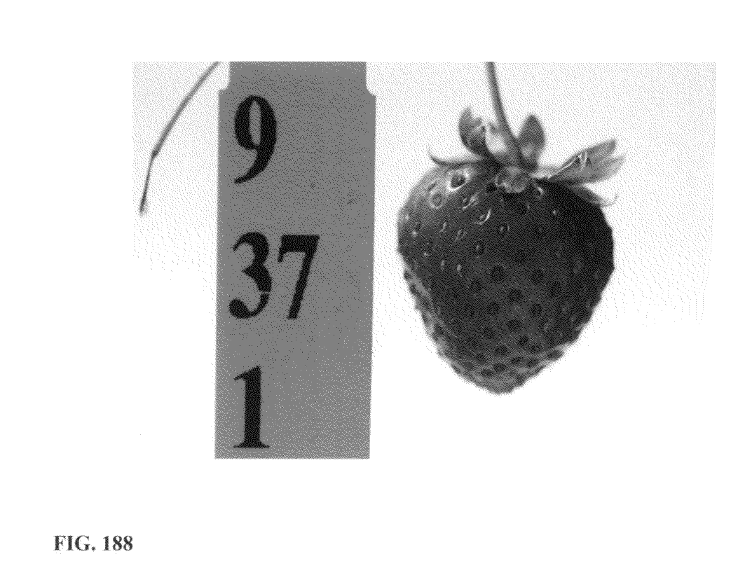

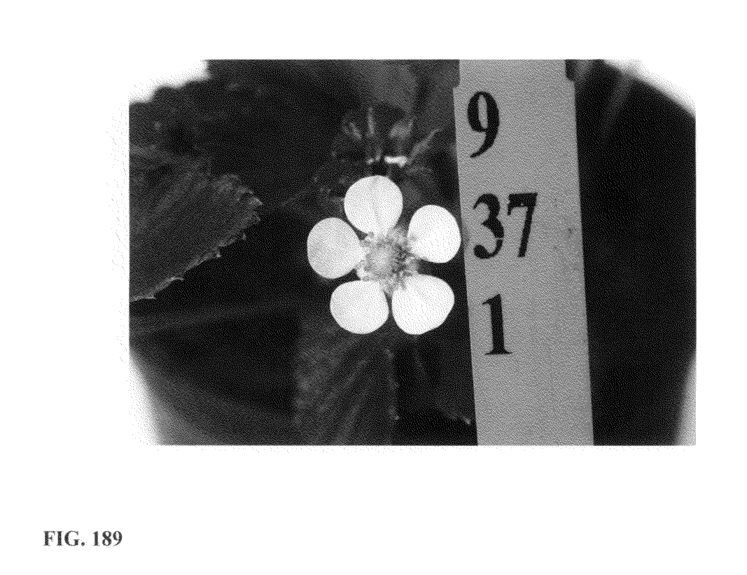

Cultivar ‘09.37-1’ has distinct disease/pathogen resistance, yield, appearance, size, and firmness. The plant of cultivar ‘09.37-1’ is characterized and distinguished in FIG. 187, the fruit of cultivar ‘09.37-1’ is characterized and distinguished in FIG. 188, and the flower of cultivar ‘09.37-1’ is characterized and distinguished in FIG. 189.

The following detailed description of cultivar ‘09.37-1’ is based on observations taken of plants and fruits (see FIGS. 187 and 188).

Certain characteristics of cultivar ‘09.37-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 63 provides data that characterizes the disease/pathogen resistance, yield, appearance, size, and firmness of cultivar ‘09.37-1’.

TABLE 63

| Cultivar | Vert | Phy | Fus | Mac | Yield | App | Size | Firm |
|---|---|---|---|---|---|---|---|---|
| 09.37-1 | 4.3 | 3.6 | 4.5 | 4.5 | 1993 | 3.8 | 36.5 | 10.8 |

### Cultivar ‘09.48-3’

In certain aspects, the present invention relates to a new and distinctive day-neutral type strawberry cultivar designated as ‘09.48-3’. Cultivar ‘09.48-3’ is typical of day-neutral strawberry cultivars and produces fruit regardless of day length when treated appropriately in arid, subtropical climates.

The strawberry cultivar ‘09.48-3’ originated from a cross between two unreleased germplasm accessions ‘04.41-7’ and ‘05.197-2’. The cultivar has been asexually reproduced by runners. The properties of this cultivar were found to be transmissible by such asexual reproduction. The cultivar is stable and reproduces true to type in successive generations of asexual reproduction.

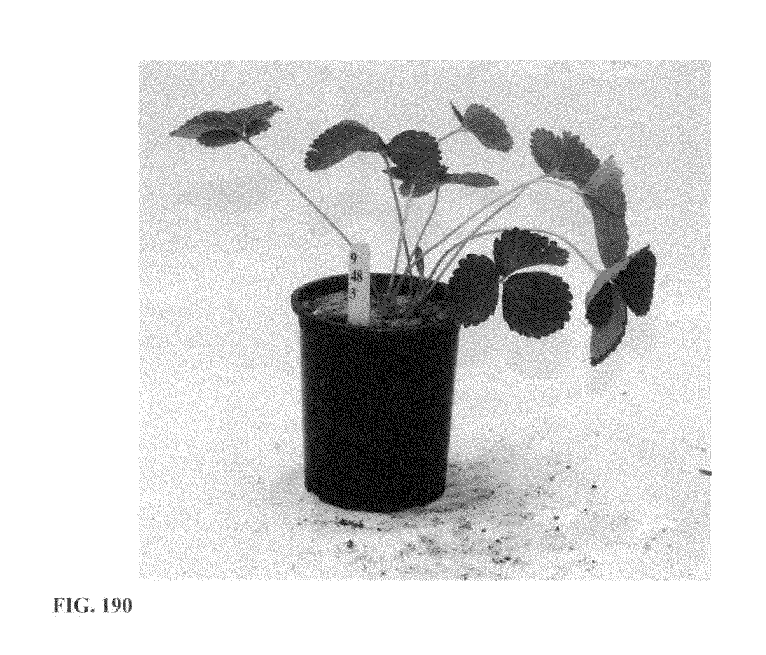

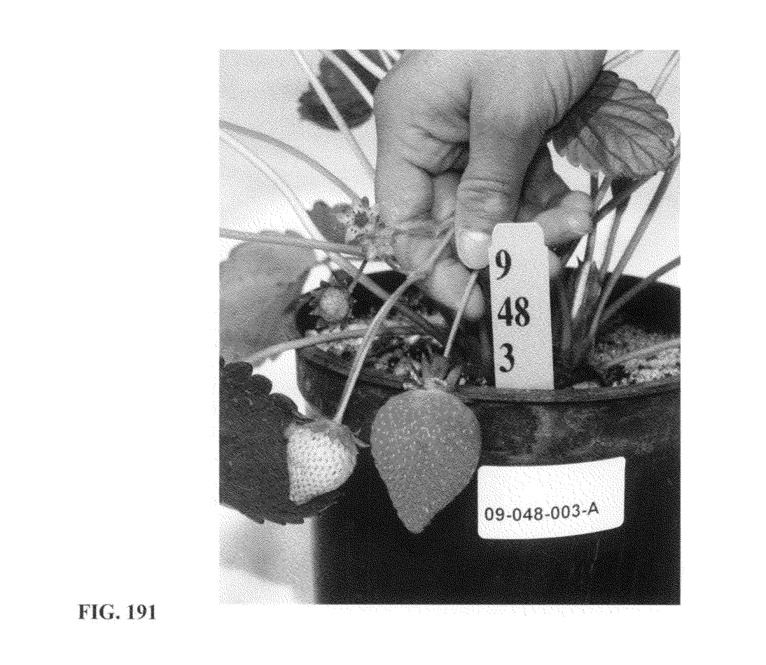

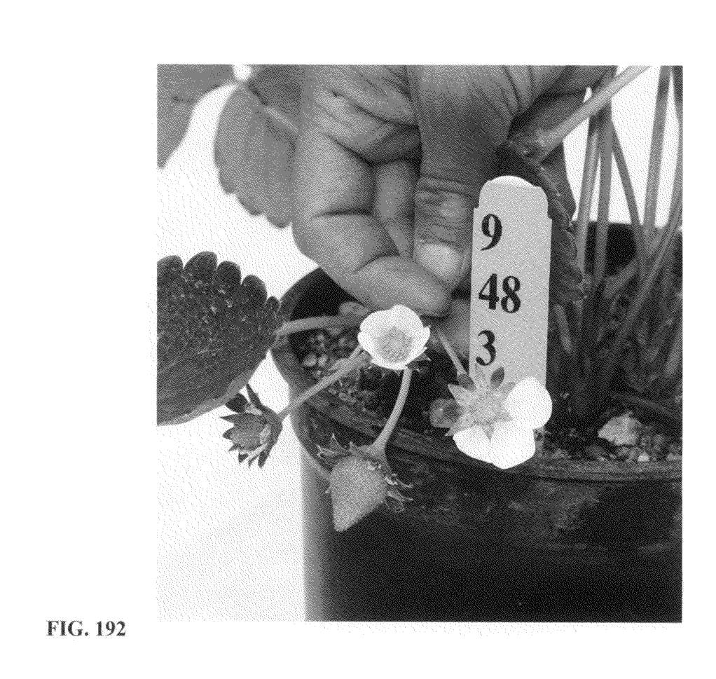

Cultivar ‘09.48-3’ has distinct disease/pathogen resistance, yield, appearance, size, and firmness. The plant of cultivar ‘09.48-3’ is characterized and distinguished in FIG. 190, the fruit of cultivar ‘09.48-3’ is characterized and distinguished in FIG. 191, and the flower of cultivar ‘09.48-3’ is characterized and distinguished in FIG. 192.

The following detailed description of cultivar ‘09.48-3’ is based on observations taken of plants and fruits (see FIGS. 190 and 191).

Certain characteristics of cultivar ‘09.48-3’ may change with changing environmental conditions (e.g., light, temperature, moisture, etc.), nutrient availability, or other factors. Quantified measurements are expressed as an average of measurements taken from a number of individual plants of the new Cultivar. The measurements of any individual plant, or any group of plants, of the new cultivar may vary from the stated average.

Table 64 provides data that characterizes the yield, appearance, size, and firmness of cultivar ‘09.48-3’.

TABLE 64

| Cultivar | Yield | App | Size | Firm |
|---|---|---|---|---|
| 09.48-3 | 2520 | 3.7 | 31.6 | 11.1 |

### Cultivar ‘09.56-608’

In certain aspects, the present invention relates to a new and distinctive short-day type strawberry cultivar designated as ‘09.56-608’. Cultivar ‘09.56-608’ is typical of short-day strawberry cultivars and produces fruit over an extended period when treated appropriately in arid, subtropical climates.

The strawberry cultivar ‘09.56-608’ originated from a cross between two unreleased germplasm accessions ‘04.45-605’ and ‘06.137-2’. The cultivar has been asexually reproduced by runners. The properties of this cultivar were found to be transmissible by such asexual reproduction. The cultivar is stable and reproduces true to type in successive generations of asexual reproduction.

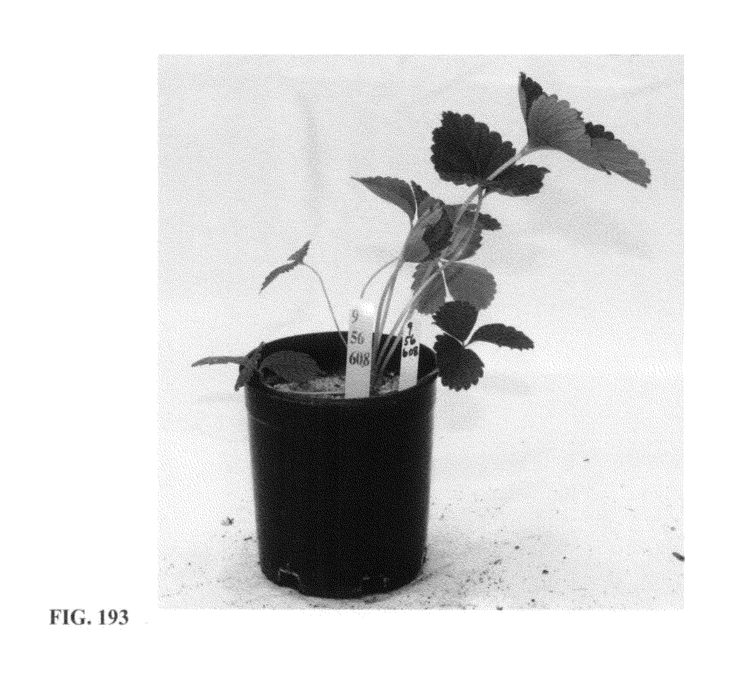

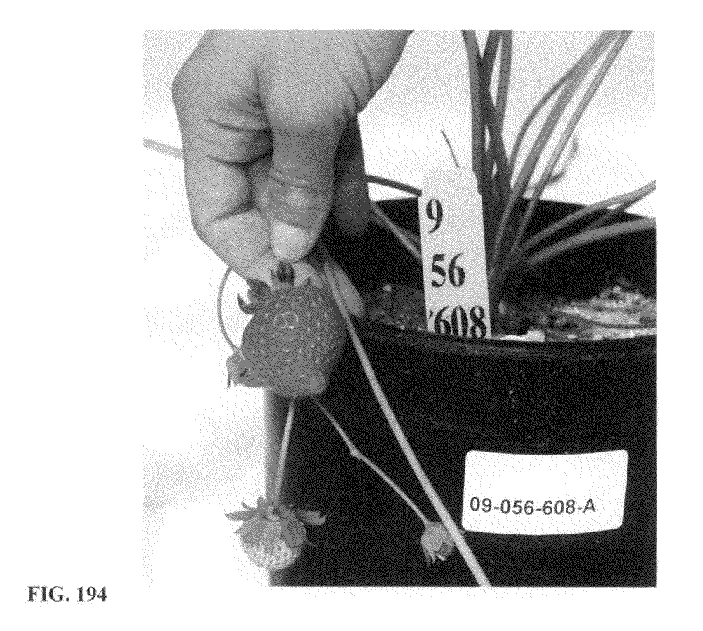

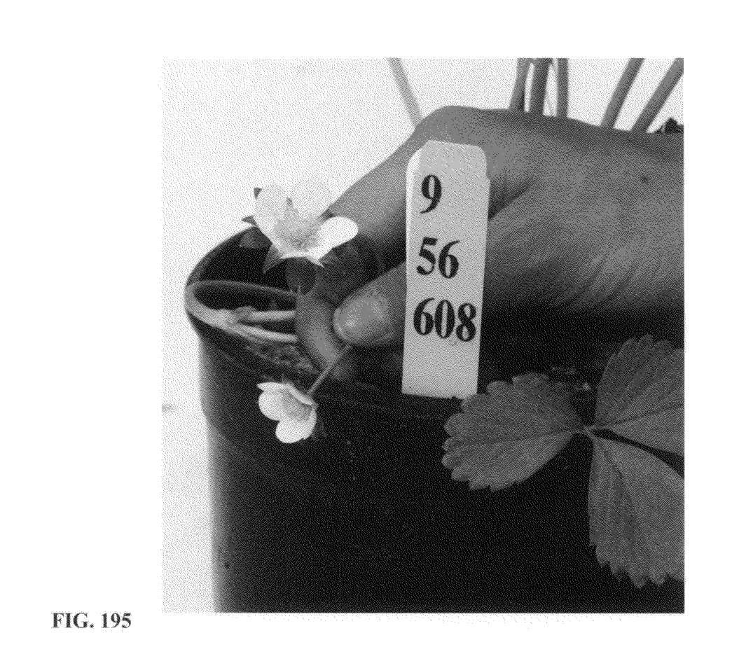

Cultivar ‘09.56-608’ has distinct disease/pathogen resistance, yield, appearance, size, and firmness. The plant of cultivar ‘09.56-608’ is characterized and distinguished in FIG. 193, the fruit of cultivar ‘09.56-608’ is characterized and distinguished in FIG. 194, and the flower of cultivar ‘09.56-608’ is characterized and distinguished in FIG. 195.

The following detailed description of cultivar ‘09.56-608’ is based on observations taken of plants and fruits (see FIGS. 193 and 194).

Certain characteristics of cultivar ‘09.56-608’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 65 provides data that characterizes the yield, appearance, size, and firmness of cultivar ‘09.56-608’.

TABLE 65

| Cultivar | Yield | App | Size | Firm |
|---|---|---|---|---|
| 09.56-608 | 1724 | 3.2 | 27.3 | 10.0 |

### Cultivar ‘09.61-602’

In certain aspects, the present invention relates to a new and distinctive short-day type strawberry cultivar designated as ‘09.61-602’. Cultivar ‘09.61-602’ is typical of short-day strawberry cultivars and produces fruit over an extended period when treated appropriately in arid, subtropical climates.

The strawberry cultivar ‘09.61-602’ originated from a cross between two unreleased germplasm accessions ‘05.154-2’ and ‘04.76-4’. The cultivar has been asexually reproduced by runners. The properties of this cultivar were found to be transmissible by such asexual reproduction. The cultivar is stable and reproduces true to type in successive generations of asexual reproduction.

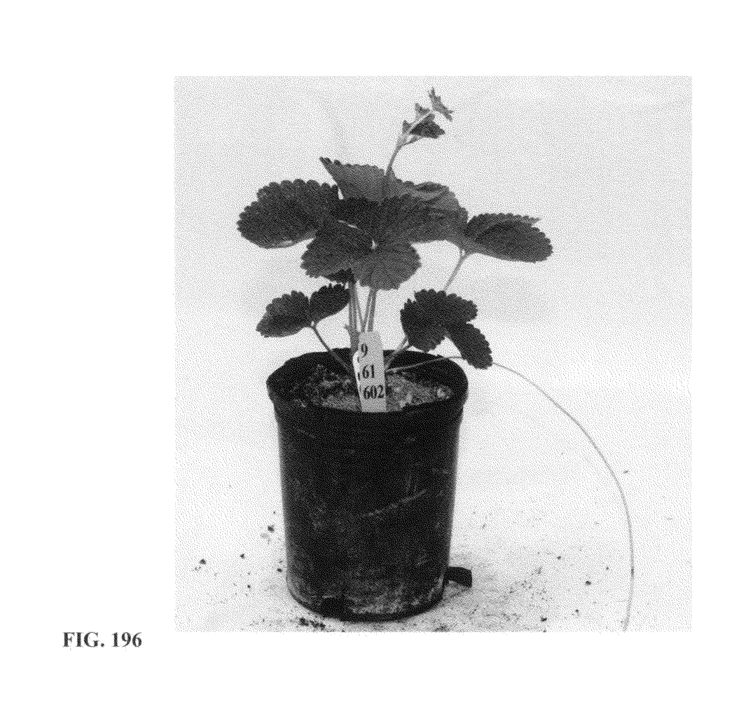

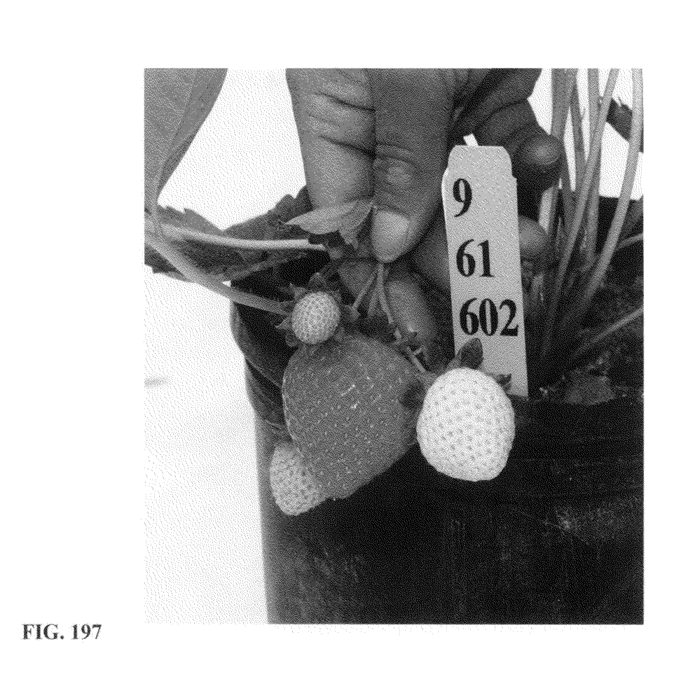

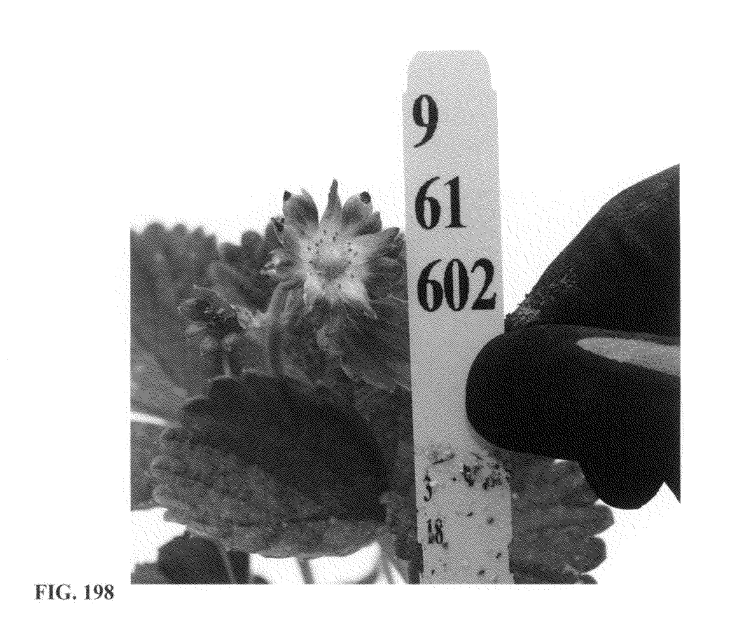

Cultivar ‘09.61-602’ has distinct disease/pathogen resistance, yield, appearance, size, and firmness. The plant of cultivar ‘09.61-602’ is characterized and distinguished in FIG. 196, the fruit of cultivar ‘09.61-602’ is characterized and distinguished in FIG. 197, and the flower of cultivar ‘09.61-602’ is characterized and distinguished in FIG. 198.

The following detailed description of cultivar ‘09.61-602’ is based on observations taken of plants and fruits (see FIGS. 196 and 197).

Certain characteristics of cultivar ‘09.61-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 66 provides data that characterizes the disease/pathogen resistance, yield, appearance, size, and firmness of cultivar ‘09.61-602’.

TABLE 66

| Cultivar | Vert | Phy | Fus | Yield | App | Size | Firm |
|---|---|---|---|---|---|---|---|
| 09.61-602 | 3.7 | 3.7 | 5.0 | 2833 | 3.6 | 30.1 | 10.8 |

### Cultivar ‘09.62-1’

In certain aspects, the present invention relates to a new and distinctive short-day type strawberry cultivar designated as ‘09.62-1’. Cultivar ‘09.62-1’ is typical of short-day strawberry cultivars and produces fruit over an extended period when treated appropriately in arid, subtropical climates.

The strawberry cultivar ‘09.62-1’ originated from a cross between two unreleased germplasm accessions ‘05.165-1’ and ‘04.76-4’. The cultivar has been asexually reproduced by runners. The properties of this cultivar were found to be transmissible by such asexual reproduction. The cultivar is stable and reproduces true to type in successive generations of asexual reproduction.

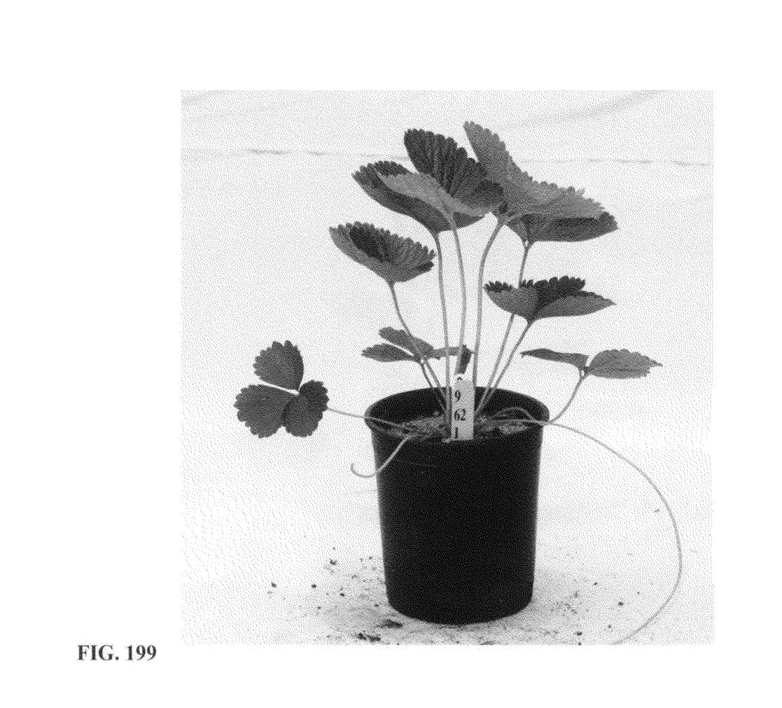

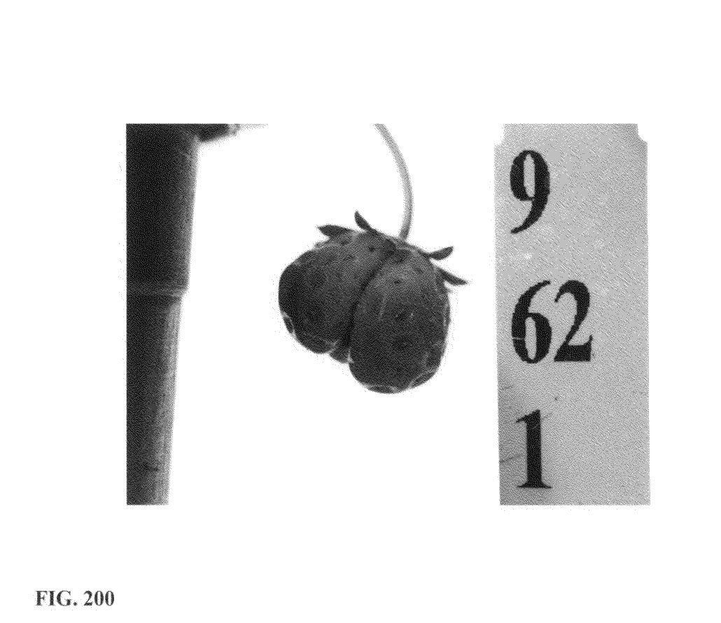

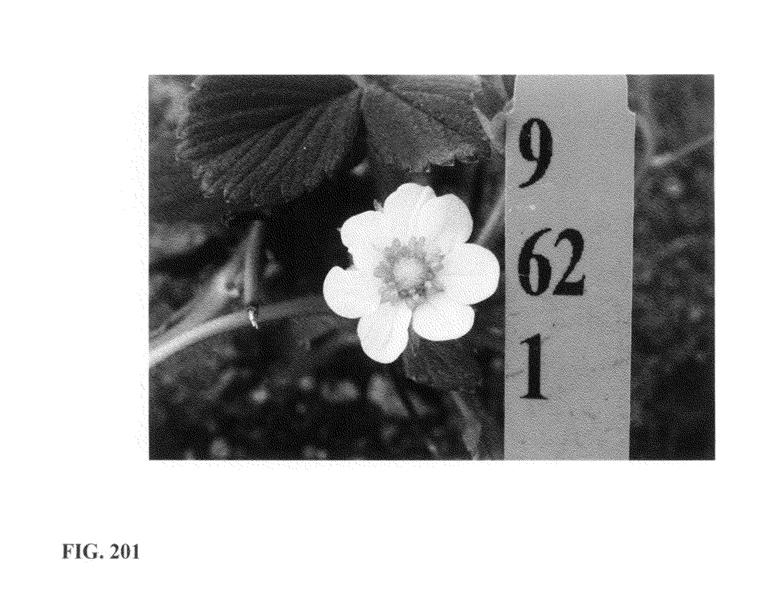

Cultivar ‘09.62-1’ has distinct disease/pathogen resistance, yield, appearance, size, and firmness. The plant of cultivar ‘09.62-1’ is characterized and distinguished in FIG. 199, the fruit of cultivar ‘09.62-1’ is characterized and distinguished in FIG. 200, and the flower of cultivar ‘09.62-1’ is characterized and distinguished in FIG. 201.

The following detailed description of cultivar ‘09.62-1’ is based on observations taken of plants and fruits (see FIGS. 199 and 200).

Certain characteristics of cultivar ‘09.62-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 67 provides data that characterizes the disease/pathogen resistance, yield, appearance, size, and firmness of cultivar ‘09.62-1’.

TABLE 67

| Cultivar | Vert | Phy | Fus | Yield | App | Size | Firm |
|---|---|---|---|---|---|---|---|
| 09.62-1 | 4.2 | 4.5 | 4.4 | 2177 | 3.8 | 33.6 | 11.4 |

### Cultivar ‘09.68-602’

In certain aspects, the present invention relates to a new and distinctive short-day type strawberry cultivar designated as ‘09.68-602’. Cultivar ‘09.68-602’ is typical of short-day strawberry cultivars and produces fruit over an extended period when treated appropriately in arid, subtropical climates.

The strawberry cultivar ‘09.68-602’ originated from a cross between two unreleased germplasm accessions ‘05.165-1’ and ‘05.92-11’. The cultivar has been asexually reproduced by runners. The properties of this cultivar were found to be transmissible by such asexual reproduction. The cultivar is stable and reproduces true to type in successive generations of asexual reproduction.

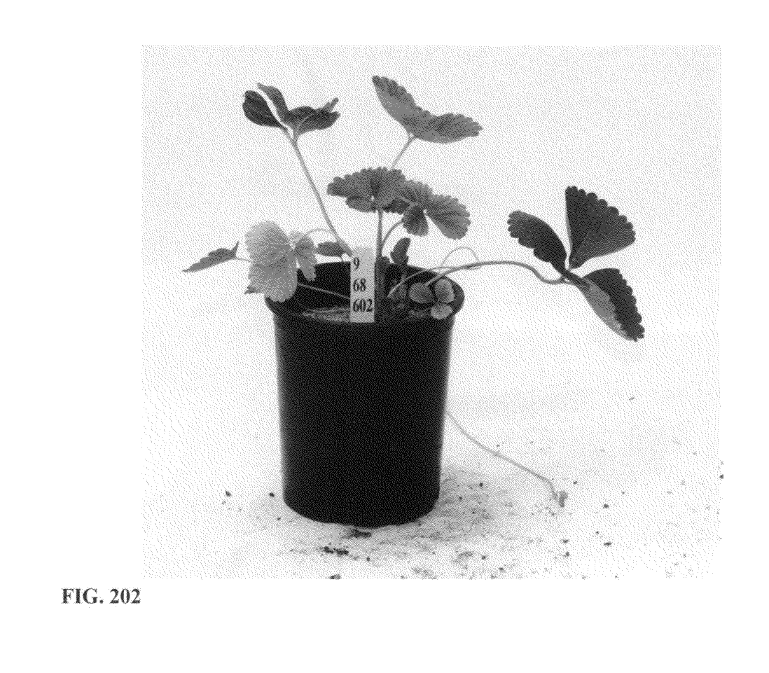

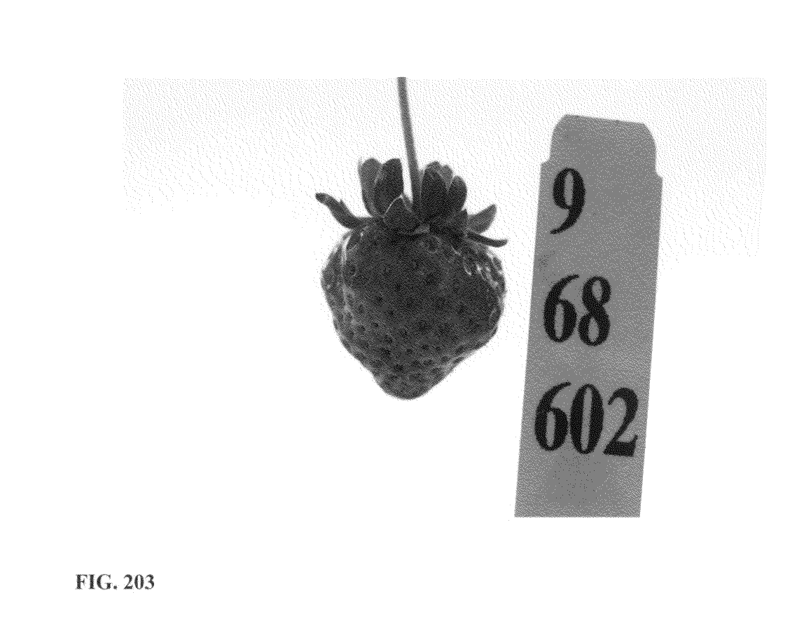

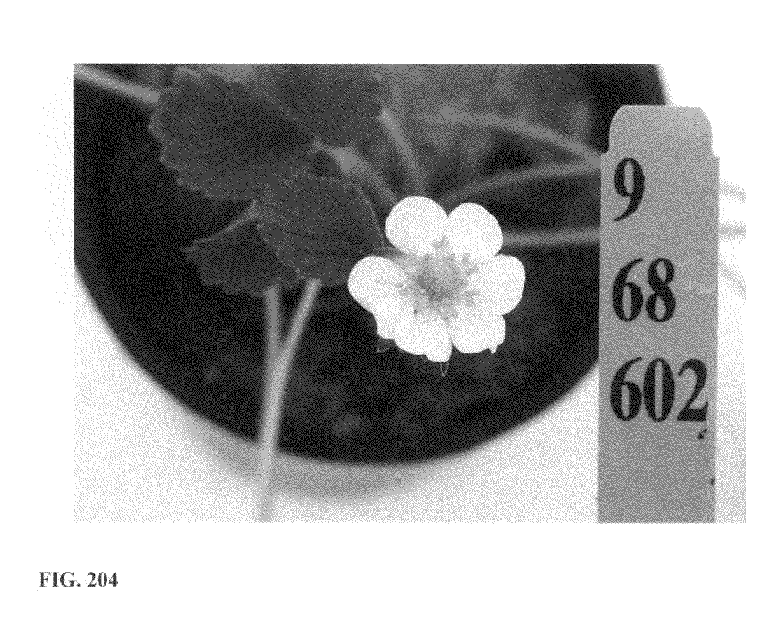

Cultivar ‘09.68-602’ has distinct disease/pathogen resistance, yield, appearance, size, and firmness. The plant of cultivar ‘09.68-602’ is characterized and distinguished in FIG. 202, the fruit of cultivar ‘09.68-602’ is characterized and distinguished in FIG. 203, and the flower of cultivar ‘09.68-602’ is characterized and distinguished in FIG. 204.

The following detailed description of cultivar ‘09.68-602’ is based on observations taken of plants and fruits (see FIGS. 202 and 203).

Certain characteristics of cultivar ‘09.68-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 68 provides data that characterizes the disease/pathogen resistance, yield, appearance, size, and firmness of cultivar ‘09.68-602’.

TABLE 68

| Cultivar | Vert | Phy | Fus | Mac | Yield | App | Size | Firm |
|---|---|---|---|---|---|---|---|---|
| 09.68-602 | 3.7 | 2.7 | 4.4 | 4.9 | 2354 | 3.9 | 33.9 | 11.0 |

### Cultivar ‘09.78-603’

In certain aspects, the present invention relates to a new and distinctive short-day type strawberry cultivar designated as ‘09.78-603’. Cultivar ‘09.78-603’ is typical of short-day strawberry cultivars and produces fruit over an extended period when treated appropriately in arid, subtropical climates.

The strawberry cultivar ‘09.78-603’ originated from a cross between two unreleased germplasm accessions ‘05.117-5’ and ‘05.144-3’. The cultivar has been asexually reproduced by runners. The properties of this cultivar were found to be transmissible by such asexual reproduction. The cultivar is stable and reproduces true to type in successive generations of asexual reproduction.

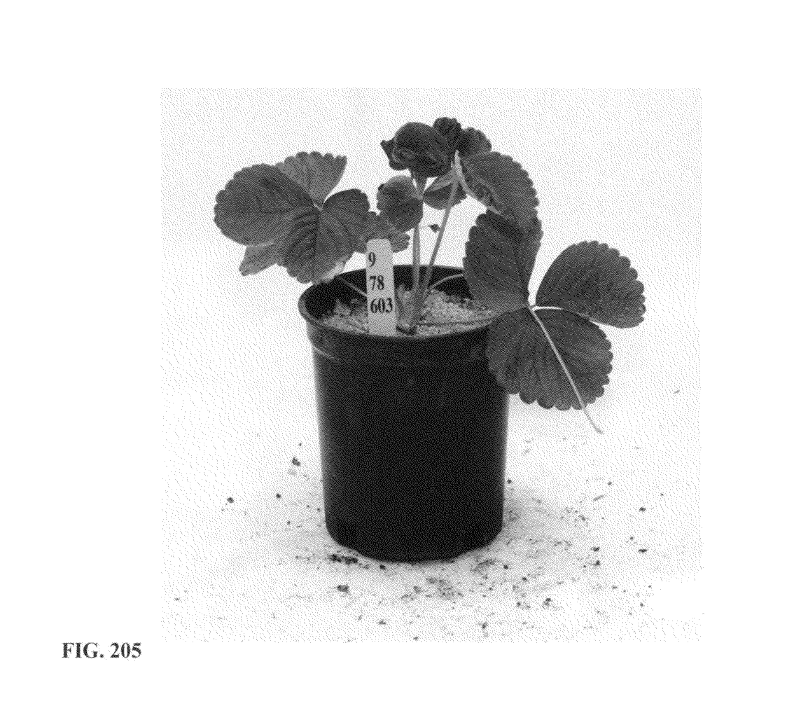

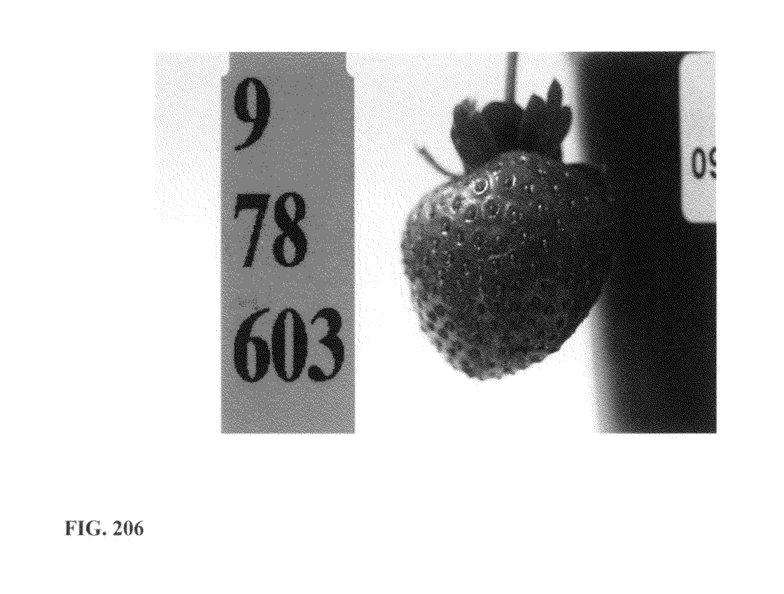

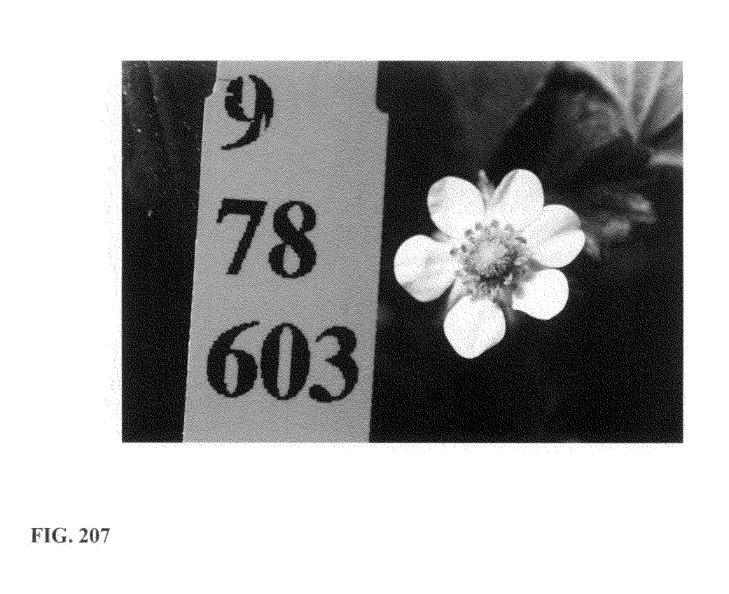

Cultivar ‘09.78-603’ has distinct disease/pathogen resistance, yield, appearance, size, and firmness. The plant of cultivar ‘09.78-603’ is characterized and distinguished in FIG. 205, the fruit of cultivar ‘09.78-603’ is characterized and distinguished in FIG. 206, and the flower of cultivar ‘09.78-603’ is characterized and distinguished in FIG. 207.

The following detailed description of cultivar ‘09.78-603’ is based on observations taken of plants and fruits (see FIGS. 205 and 206).

Certain characteristics of cultivar ‘09.78-603’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 69 provides data that characterizes the yield, appearance, size, and firmness of cultivar ‘09.78-603’.

TABLE 69

| Cultivar | Yield | App | Size | Firm |
|---|---|---|---|---|
| 09.78-603 | 1686 | 3.4 | 32.7 | 11.4 |

### Cultivar ‘09.85-2’

In certain aspects, the present invention relates to a new and distinctive short-day type strawberry cultivar designated as ‘09.85-2’. Cultivar ‘09.85-2’ is typical of short-day strawberry cultivars and produces fruit over an extended period when treated appropriately in arid, subtropical climates.

The strawberry cultivar ‘09.85-2’ originated from a cross between two unreleased germplasm accessions ‘05.154-2’ and ‘05.172-2’. The cultivar has been asexually reproduced by runners. The properties of this cultivar were found to be transmissible by such asexual reproduction. The cultivar is stable and reproduces true to type in successive generations of asexual reproduction.

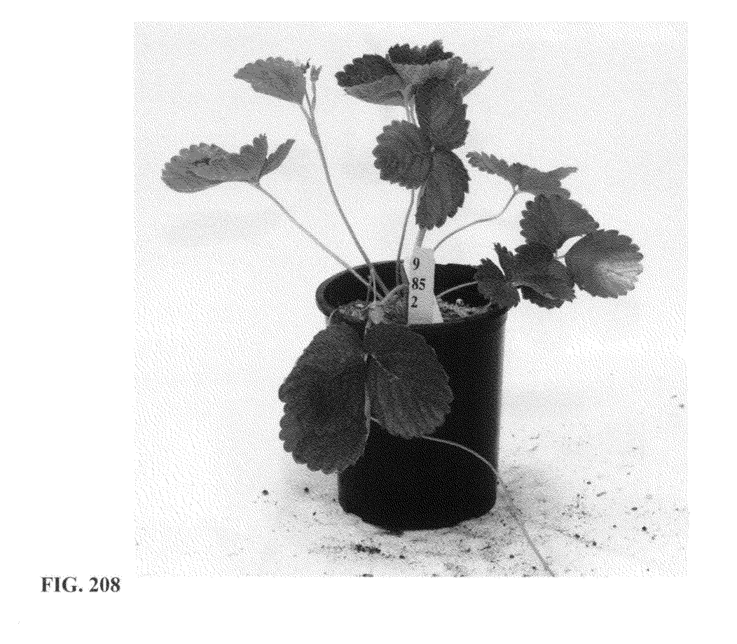

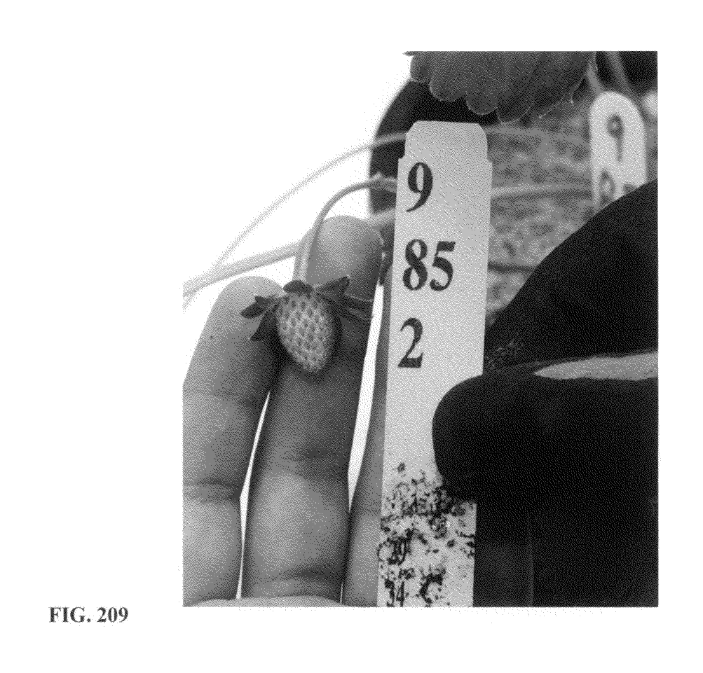

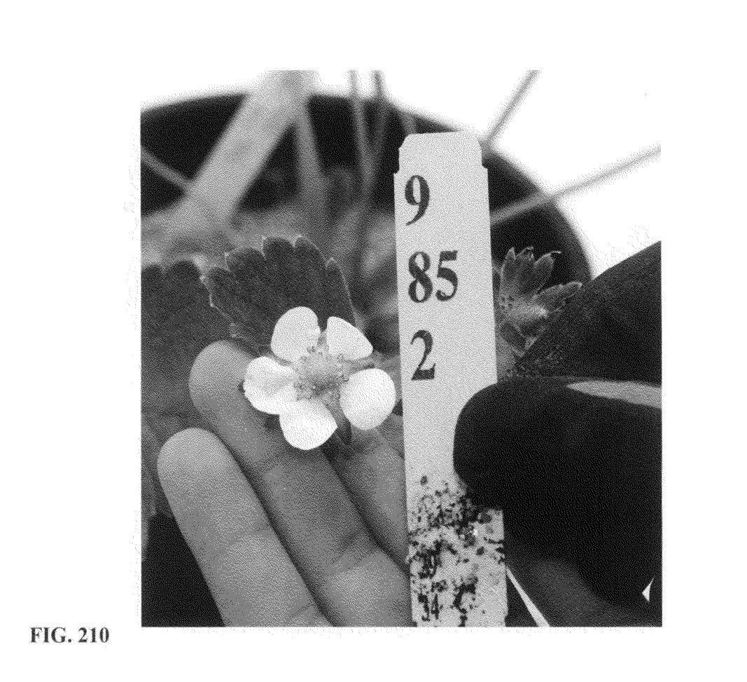

Cultivar ‘09.85-2’ has distinct disease/pathogen resistance, yield, appearance, size, and firmness. The plant of cultivar ‘09.85-2’ is characterized and distinguished in FIG. 208, the fruit of cultivar ‘09.85-2’ is characterized and distinguished in FIG. 209, and the flower of cultivar ‘09.85-2’ is characterized and distinguished in FIG. 210.

The following detailed description of cultivar ‘09.85-2’ is based on observations taken of plants and fruits (see FIGS. 208 and 209).

Certain characteristics of cultivar ‘09.85-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 70 provides data that characterizes the disease/pathogen resistance, yield, appearance, size, and firmness of cultivar ‘09.85-2’.

TABLE 70

| Cultivar | Vert | Phy | Fus | Mac | Yield | App | Size | Firm |
|---|---|---|---|---|---|---|---|---|
| 09.85-2 | 3.2 | 4.7 | 3.3 | 2.6 | 2537 | 3.9 | 36.2 | 9.9 |

### Cultivar ‘09.85-601’

In certain aspects, the present invention relates to a new and distinctive short-day type strawberry cultivar designated as ‘09.85-601’. Cultivar ‘09.85-601’ is typical of short-day strawberry cultivars and produces fruit over an extended period when treated appropriately in arid, subtropical climates.

The strawberry cultivar ‘09.85-601’ originated from a cross between two unreleased germplasm accessions ‘05.154-2’ and ‘05.172-2’. The cultivar has been asexually reproduced by runners. The properties of this cultivar were found to be transmissible by such asexual reproduction. The cultivar is stable and reproduces true to type in successive generations of asexual reproduction.

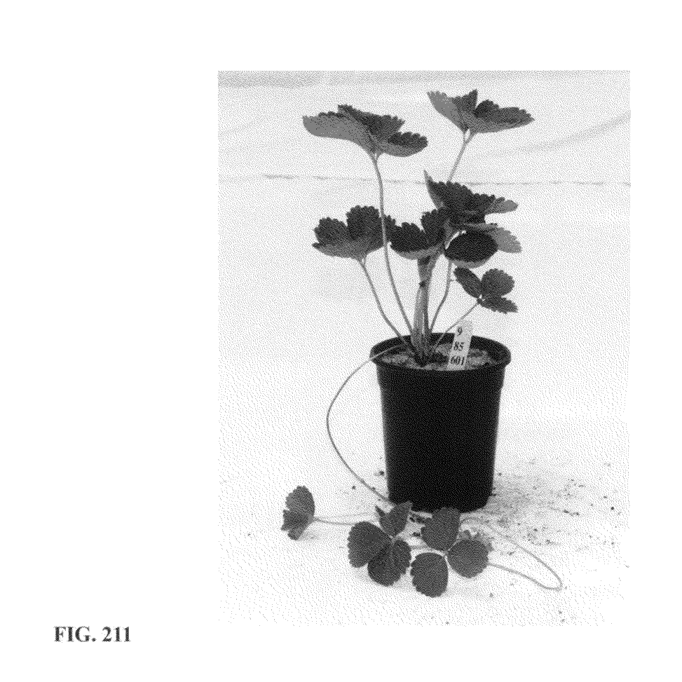

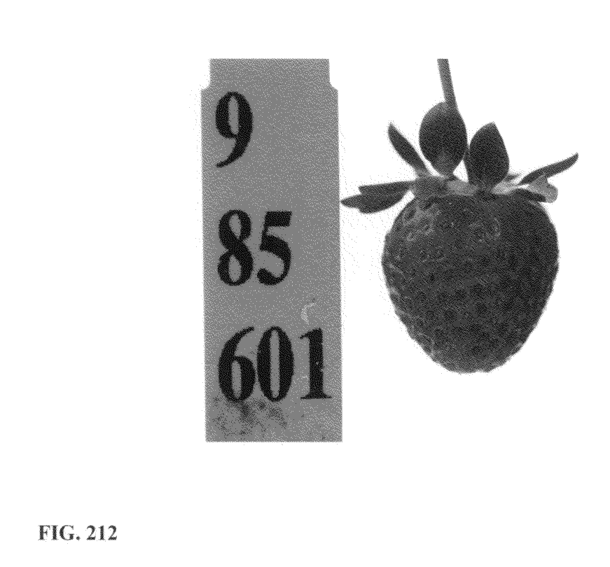

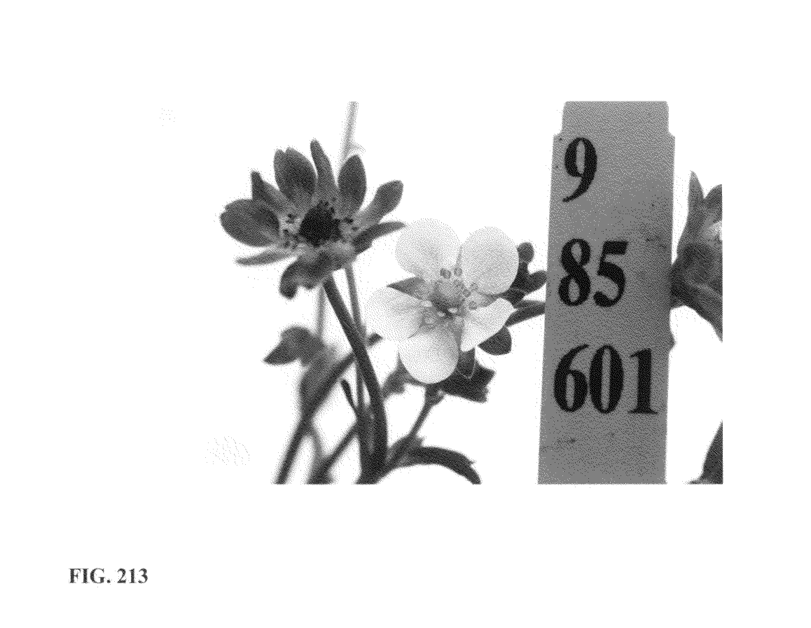

Cultivar ‘09.85-601’ has distinct disease/pathogen resistance, yield, appearance, size, and firmness. The plant of cultivar ‘09.85-601’ is characterized and distinguished in FIG. 211, the fruit of cultivar ‘09.85-601’ is characterized and distinguished in FIG. 212, and the flower of cultivar ‘09.85-601’ is characterized and distinguished in FIG. 213.

The following detailed description of cultivar ‘09.85-601’ is based on observations taken of plants and fruits (see FIGS. 211 and 212).

Certain characteristics of cultivar ‘09.85-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 71 provides data that characterizes the yield, appearance, size, and firmness of cultivar ‘09.85-601’.

TABLE 71

| Cultivar | Yield | App | Size | Firm |
|---|---|---|---|---|
| 09.85-601 | 2142 | 4.0 | 33.8 | 10.4 |

### Cultivar ‘09.88-602’

In certain aspects, the present invention relates to a new and distinctive short-day type strawberry cultivar designated as ‘09.88-602’. Cultivar ‘09.88-602’ is typical of short-day strawberry cultivars and produces fruit over an extended period when treated appropriately in arid, subtropical climates.

The strawberry cultivar ‘09.88-602’ originated from a cross between two unreleased germplasm accessions ‘05.97-9’ and ‘05.194-1’. The cultivar has been asexually reproduced by runners. The properties of this cultivar were found to be transmissible by such asexual reproduction. The cultivar is stable and reproduces true to type in successive generations of asexual reproduction.

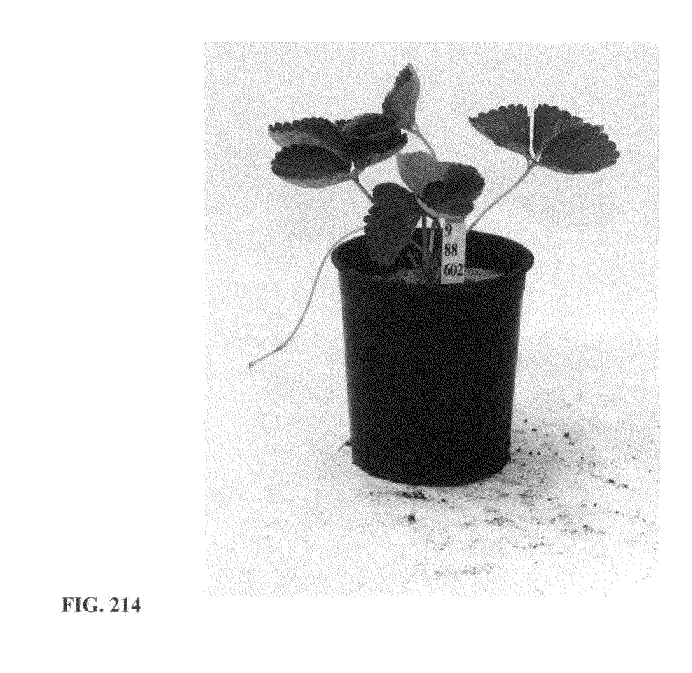

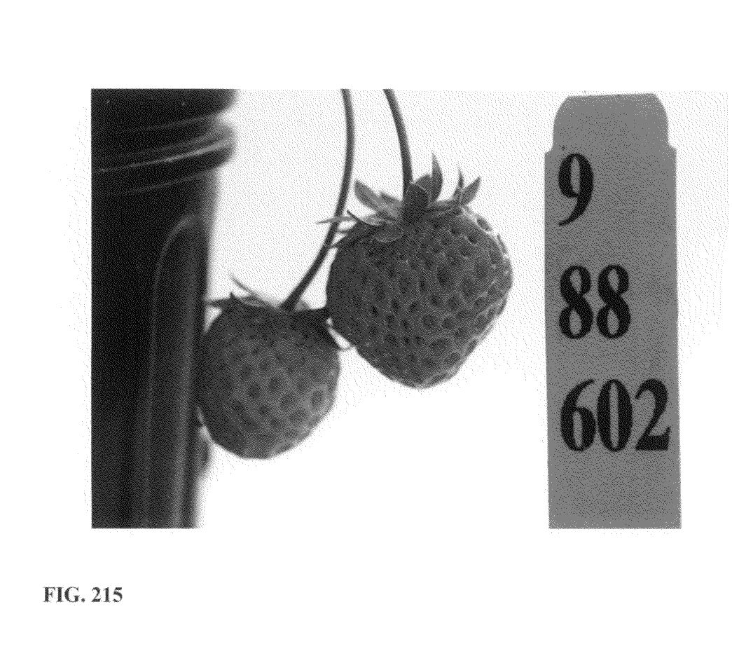

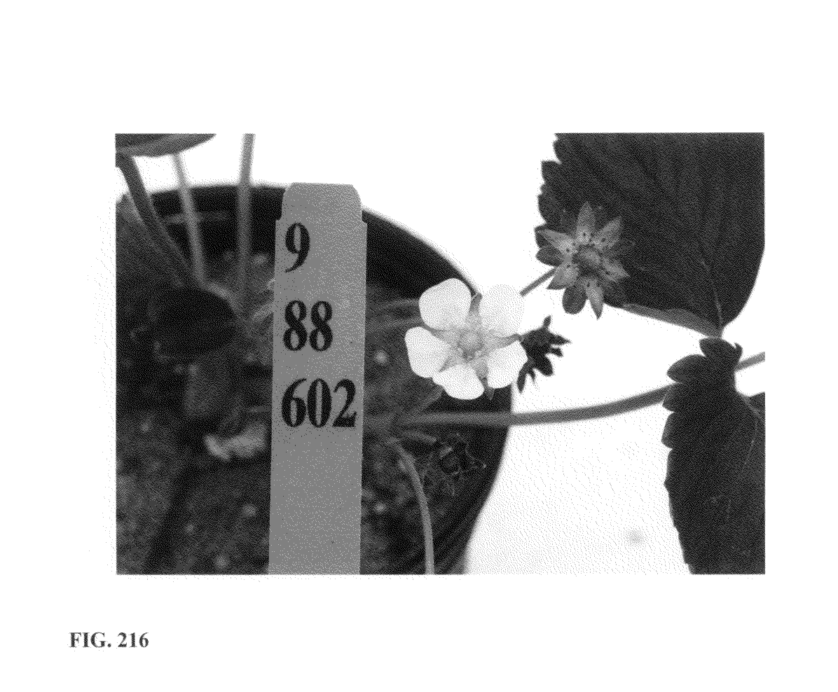

Cultivar ‘09.88-602’ has distinct disease/pathogen resistance, yield, appearance, size, and firmness. The plant of cultivar ‘09.88-602’ is characterized and distinguished in FIG. 214, the fruit of cultivar ‘09.88-602’ is characterized and distinguished in FIG. 215, and the flower of cultivar ‘09.88-602’ is characterized and distinguished in FIG. 216.

The following detailed description of cultivar ‘09.88-602’ is based on observations taken of plants and fruits (see FIGS. 214 and 215).

Certain characteristics of cultivar ‘09.88-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 72 provides data that characterizes the yield, appearance, size, and firmness of cultivar ‘09.88-602’.

TABLE 72

| Cultivar | Yield | App | Size | Firm |
|---|---|---|---|---|
| 09.88-602 | 2283 | 3.8 | 34.5 | 12.6 |

### Cultivar ‘09.91-1’

In certain aspects, the present invention relates to a new and distinctive day-neutral type strawberry cultivar designated as ‘09.91-1’. Cultivar ‘09.91-1’ is typical of day-neutral strawberry cultivars and produces fruit regardless of day length when treated appropriately in arid, subtropical climates.

The strawberry cultivar ‘09.91-1’ originated from a cross between two unreleased germplasm accessions ‘05.154-2’ and ‘05.194-1’. The cultivar has been asexually reproduced by runners. The properties of this cultivar were found to be transmissible by such asexual reproduction. The cultivar is stable and reproduces true to type in successive generations of asexual reproduction.

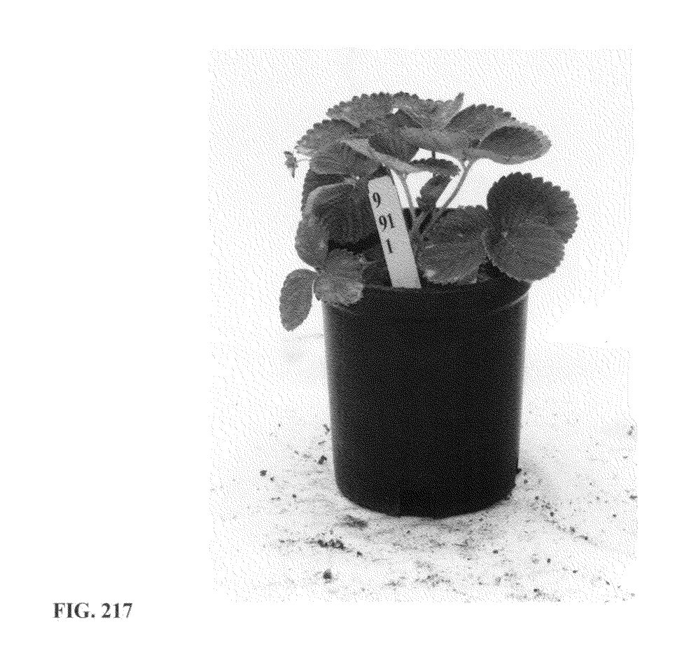

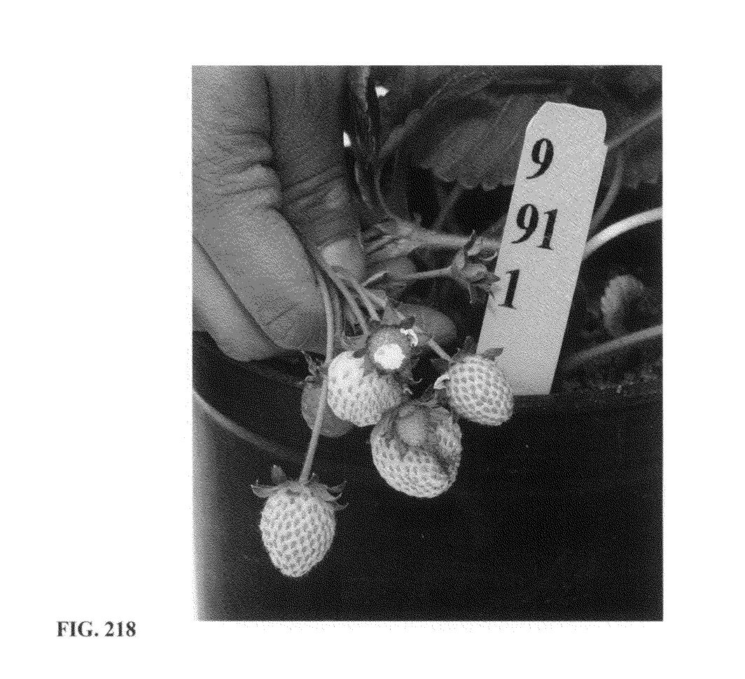

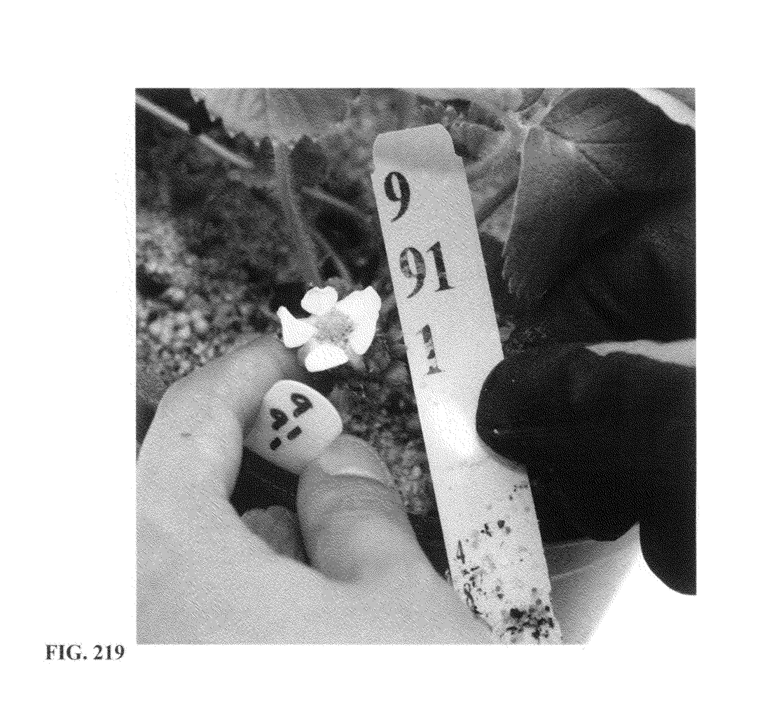

Cultivar ‘09.91-1’ has distinct disease/pathogen resistance, yield, appearance, size, and firmness. The plant of cultivar ‘09.91-1’ is characterized and distinguished in FIG. 217, the fruit of cultivar ‘09.91-1’ is characterized and distinguished in FIG. 218, and the flower of cultivar ‘09.91-1’ is characterized and distinguished in FIG. 219.

The following detailed description of cultivar ‘09.91-1’ is based on observations taken of plants and fruits (see FIGS. 217 and 218).

Certain characteristics of cultivar ‘09.91-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 73 provides data that characterizes the disease/pathogen resistance, yield, appearance, size, and firmness of cultivar ‘09.91-1’.

TABLE 73

| Cultivar | Vert | Yield | App | Size | Firm |
|---|---|---|---|---|---|
| 09.91-1 | 4.5 | 2463 | 3.7 | 28.9 | 11.2 |

### Cultivar ‘09.97-3’

In certain aspects, the present invention relates to a new and distinctive short-day type strawberry cultivar designated as ‘09.97-3’. Cultivar ‘09.97-3’ is typical of short-day strawberry cultivars and produces fruit over an extended period when treated appropriately in arid, subtropical climates.

The strawberry cultivar ‘09.97-3’ originated from a cross between two unreleased germplasm accessions ‘03.132-3’ and ‘05.194-1’. The cultivar has been asexually reproduced by runners. The properties of this cultivar were found to be transmissible by such asexual reproduction. The cultivar is stable and reproduces true to type in successive generations of asexual reproduction.

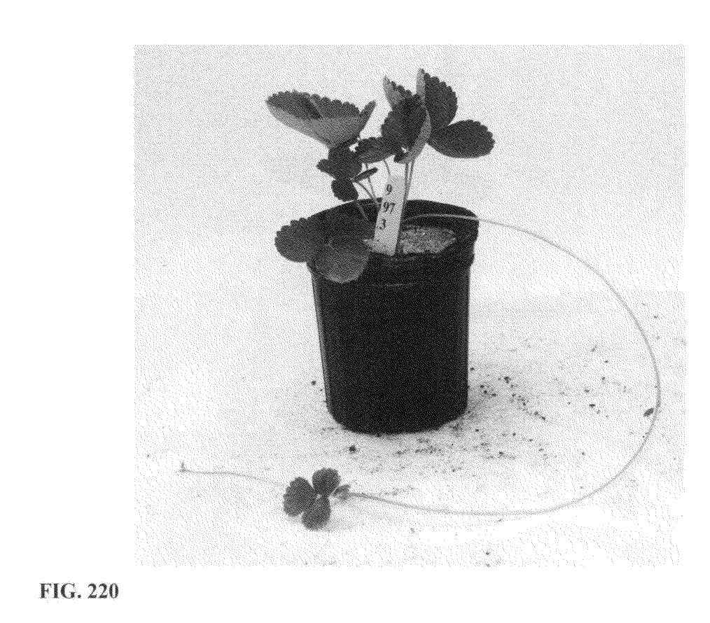

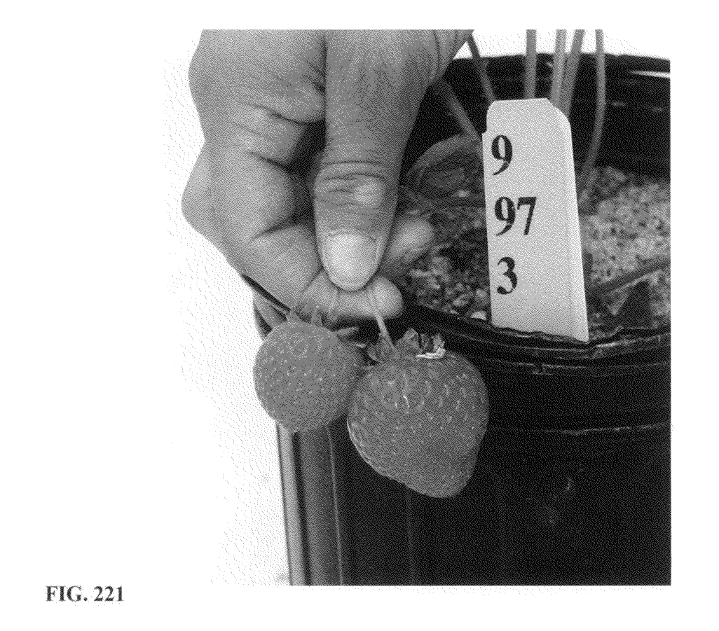

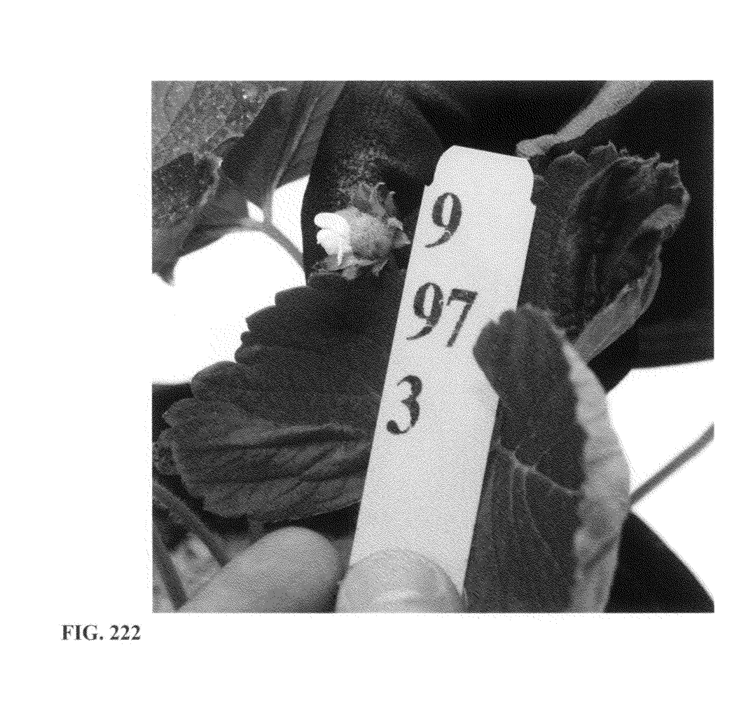

Cultivar ‘09.97-3’ has distinct disease/pathogen resistance, yield, appearance, size, and firmness. The plant of cultivar ‘09.97-3’ is characterized and distinguished in FIG. 220, the fruit of cultivar ‘09.97-3’ is characterized and distinguished in FIG. 221, and the flower of cultivar ‘09.97-3’ is characterized and distinguished in FIG. 222.

The following detailed description of cultivar ‘09.97-3’ is based on observation's taken of plants and fruits (see FIGS. 220 and 221).

Certain characteristics of cultivar ‘09.97-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 74 provides data that characterizes the disease/pathogen resistance, yield, appearance, size, and firmness of cultivar ‘09.97-3’.

TABLE 74

| Cultivar | Vert | Fus | Yield | App | Size | Firm |
|---|---|---|---|---|---|---|
| 09.97-3 | 4.7 | 4.3 | 1768 | 3.6 | 32.6 | 13.2 |

### Cultivar ‘09.100-3’

In certain aspects, the present invention relates to a new and distinctive day-neutral type strawberry cultivar designated as ‘09.100-3’. Cultivar ‘09.100-3’ is typical of day-neutral strawberry cultivars and produces fruit regardless of day length when treated appropriately in arid, subtropical climates.

The strawberry cultivar ‘09.100-3’ originated from a cross between two unreleased germplasm accessions ‘03.132-3’ and ‘05.197-2’. The cultivar has been asexually reproduced by runners. The properties of this cultivar were found to be transmissible by such asexual reproduction. The cultivar is stable and reproduces true to type in successive generations of asexual reproduction.

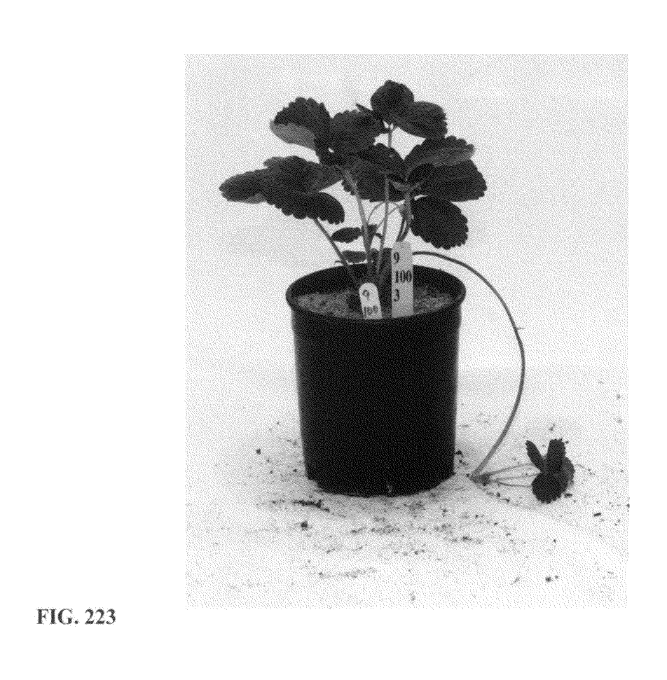

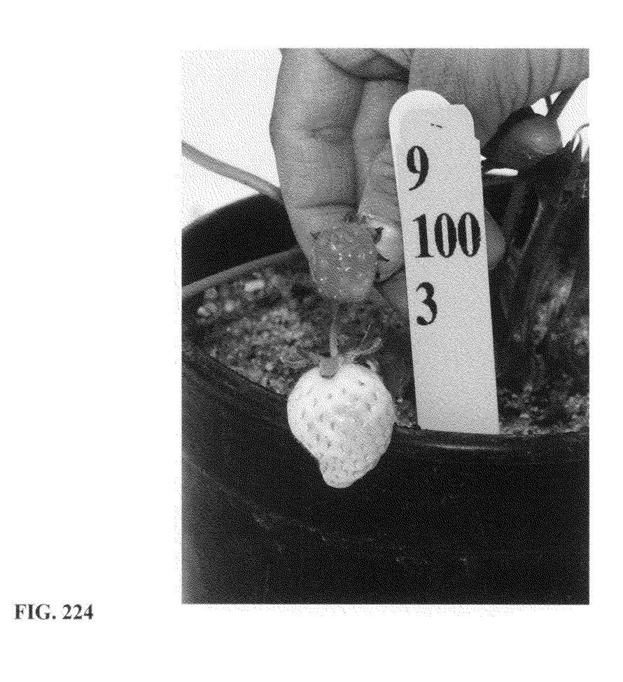

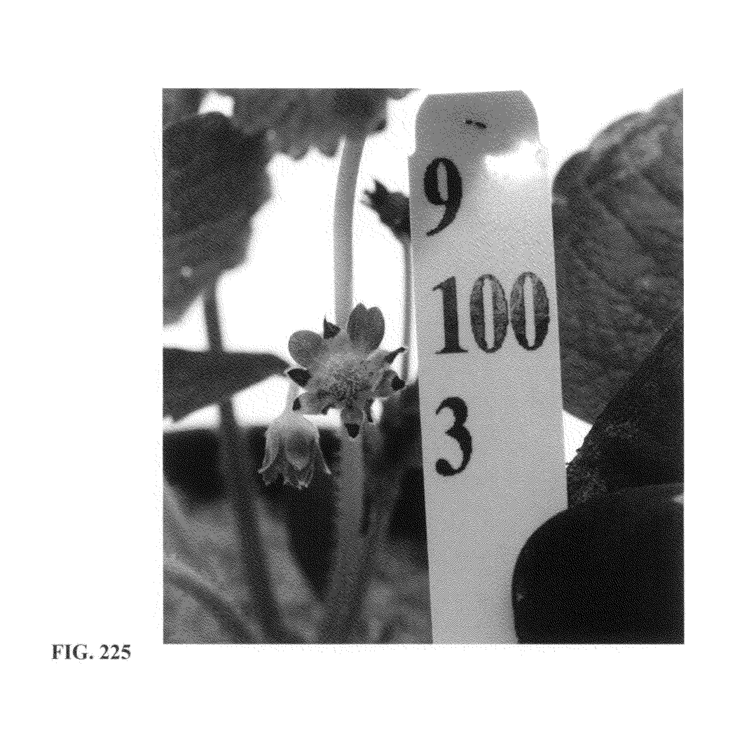

Cultivar ‘09.100-3’ has distinct disease/pathogen resistance, yield, appearance, size, and firmness. The plant of cultivar ‘09.100-3’ is characterized and distinguished in FIG. 223, the fruit of cultivar ‘09.100-3’ is characterized and distinguished in FIG. 224, and the flower of cultivar ‘09.100-3’ is characterized and distinguished in FIG. 225.

The following detailed description of cultivar ‘09.100-3’ is based on observations taken of plants and fruits (see FIGS. 223 and 224).

Certain characteristics of cultivar ‘09.100-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 75 provides data that characterizes the yield, appearance, size, and firmness of cultivar ‘09.100-3’.

TABLE 75

| Cultivar | Yield | App | Size | Firm |
|---|---|---|---|---|
| 09.100-3 | 1941 | 3.6 | 38.2 | 10.9 |

### Cultivar ‘09.103-2’

In certain aspects, the present invention relates to a new and distinctive day-neutral type strawberry cultivar designated as ‘09.103-2’. Cultivar ‘09.103-2’ is typical of day-neutral strawberry cultivars and produces fruit regardless of day length when treated appropriately in arid, subtropical climates.

The strawberry cultivar ‘09.103-2’ originated from a cross between two unreleased germplasm accessions ‘05.194-1’ and ‘05.197-2’. The cultivar has been asexually reproduced by runners. The properties of this cultivar were found to be transmissible by such asexual reproduction. The cultivar is stable and reproduces true to type in successive generations of asexual reproduction.

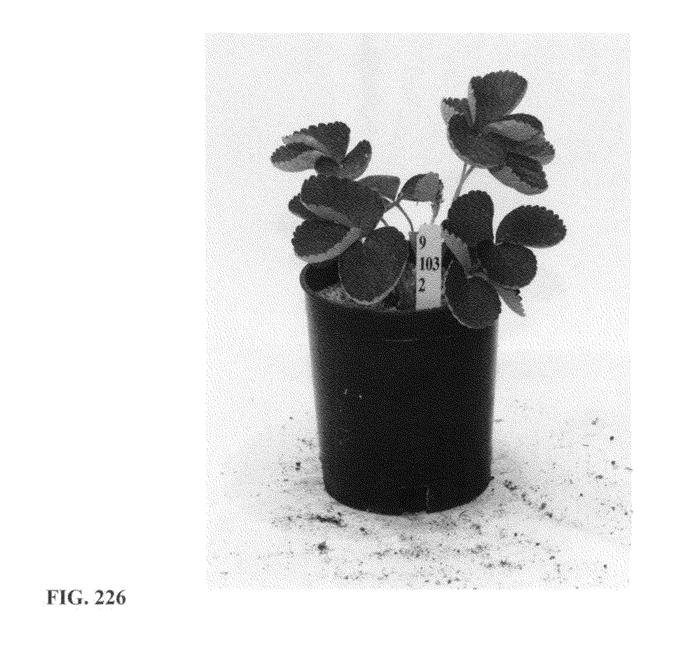

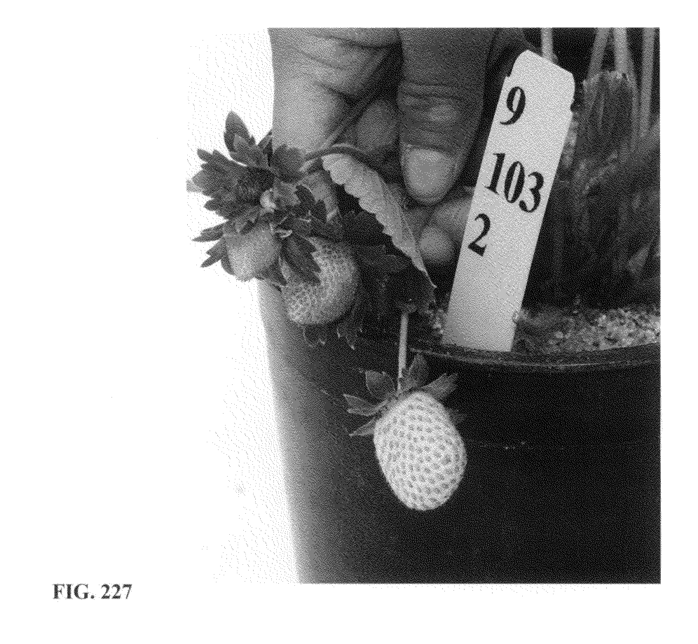

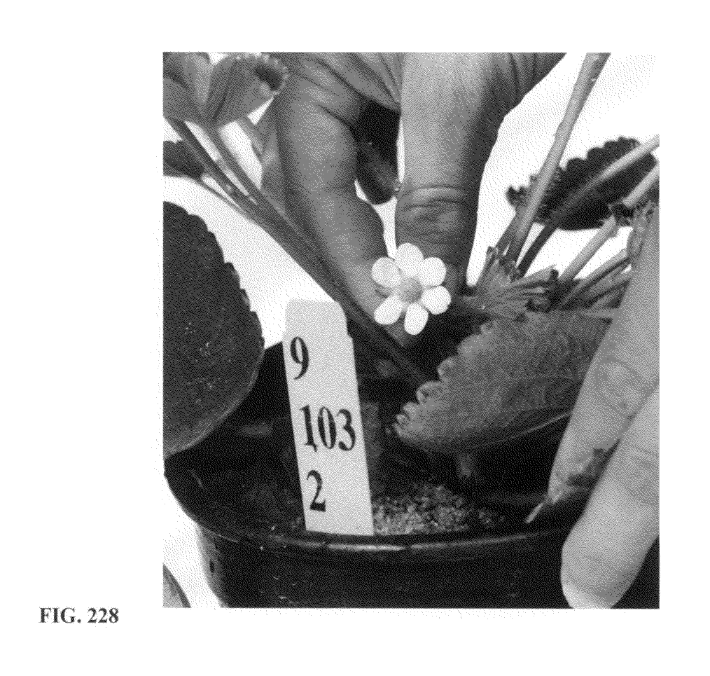

Cultivar ‘09.103-2’ has distinct disease/pathogen resistance, yield, appearance, size, and firmness. The plant of cultivar ‘09.103-2’ is characterized and distinguished in FIG. 226, the fruit of cultivar ‘09.103-2’ is characterized and distinguished in FIG. 227, and the flower of cultivar ‘09.103-2’ is characterized and distinguished in FIG. 228.

The following detailed description of cultivar ‘09.103-2’ is based on observations taken of plants and fruits (see FIGS. 226 and 227).

Certain characteristics of cultivar ‘09.103-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 76 provides data that characterizes the disease/pathogen resistance, yield, appearance, size, and firmness of cultivar ‘09.103-2’.

TABLE 76

| Cultivar | Vert | Phy | Fus | Mac | Yield | App | Size | Firm |
|---|---|---|---|---|---|---|---|---|
| 09.103-2 | 4.2 | 4.2 | 4.8 | 4.6 | 2788 | 3.4 | 32.9 | 10.4 |

### Cultivar ‘09.108-2’

In certain aspects, the present invention relates to a new and distinctive day-neutral type strawberry cultivar designated as ‘09.108-2’. Cultivar ‘09.108-2’ is typical of day-neutral strawberry cultivars and produces fruit regardless of day length when treated appropriately in arid, subtropical climates.

The strawberry cultivar ‘09.108-2’ originated from a cross between two unreleased germplasm accessions ‘05.197-2’ and ‘05.206-5’. The cultivar has been asexually reproduced by runners. The properties of this cultivar were found to be transmissible by such asexual reproduction. The cultivar is stable and reproduces true to type in successive generations of asexual reproduction.

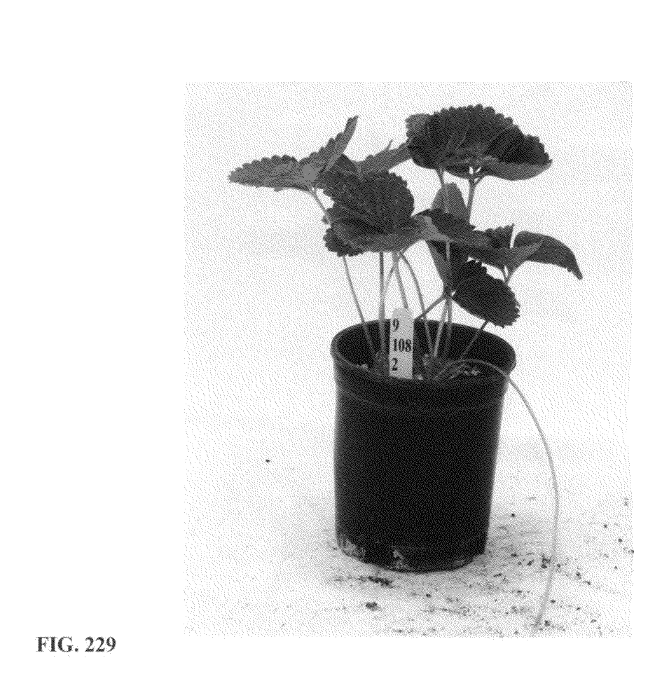

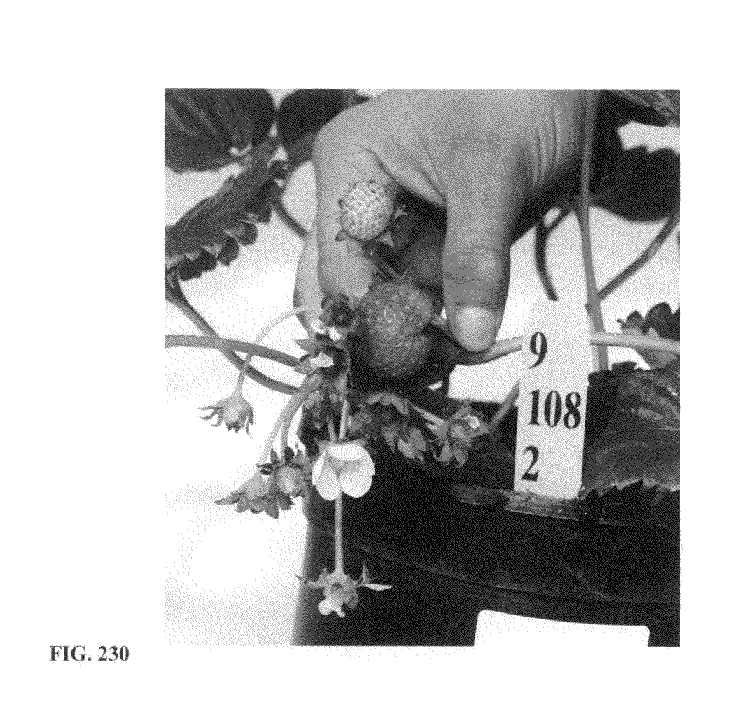

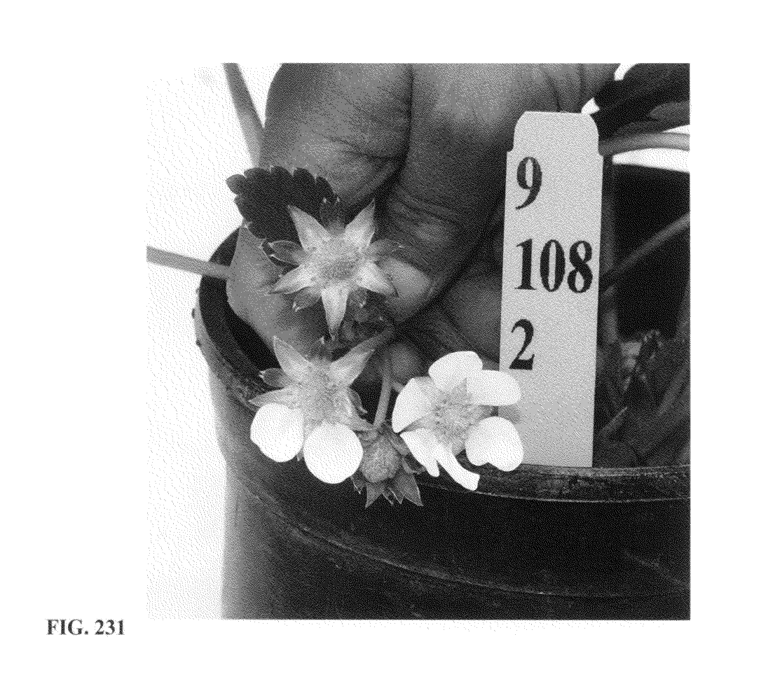

Cultivar ‘09.108-2’ has distinct disease/pathogen resistance, yield, appearance, size, and firmness. The plant of cultivar ‘09.108-2’ is characterized and distinguished in FIG. 229, the fruit of cultivar ‘09.108-2’ is characterized and distinguished in FIG. 230, and the flower of cultivar ‘09.108-2’ is characterized and distinguished in FIG. 231.

The following detailed description of cultivar ‘09.108-2’ is based on observations taken of plants and fruits (see FIGS. 229 and 230).

Certain characteristics of cultivar ‘09.108-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 77 provides data that characterizes the disease/pathogen resistance, yield, appearance, size, and firmness of cultivar ‘09.108-2’.

TABLE 77

| Cultivar | Vert | Phy | Fus | Yield | App | Size | Firm |
|---|---|---|---|---|---|---|---|
| 09.108-2 | 4.7 | 2.8 | 4.6 | 1800 | 3.9 | 32.1 | 12.6 |

### Cultivar ‘09.132-3’

In certain aspects, the present invention relates to a new and distinctive day-neutral type strawberry cultivar designated as ‘09.132-3’. Cultivar ‘09.132-3’ is typical of day-neutral strawberry cultivars and produces fruit regardless of day length when treated appropriately in arid, subtropical climates.

The strawberry cultivar ‘09.132-3’ originated from a cross between two unreleased germplasm accessions ‘05.197-2’ and ‘05.107-2’. The cultivar has been asexually reproduced by runners. The properties of this cultivar were found to be transmissible by such asexual reproduction. The cultivar is stable and reproduces true to type in successive generations of asexual reproduction.

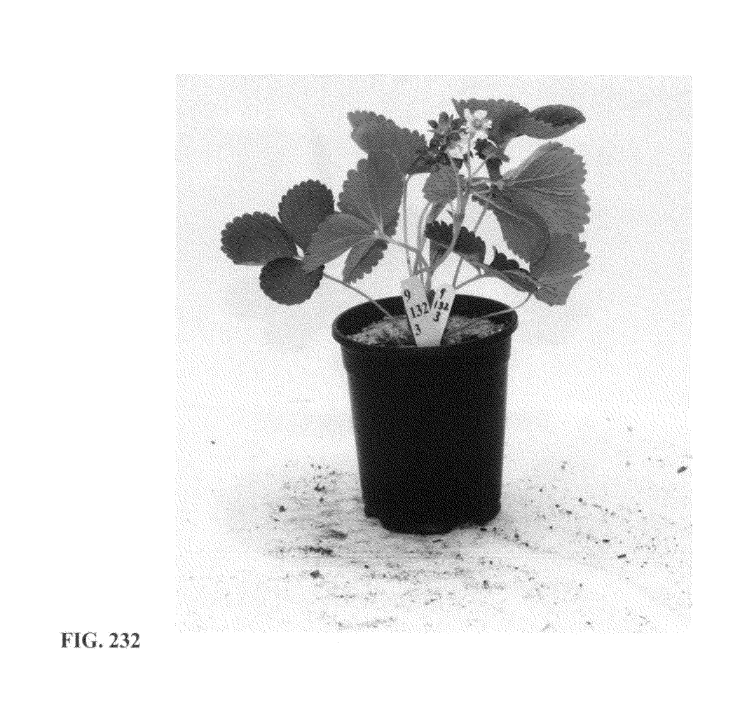

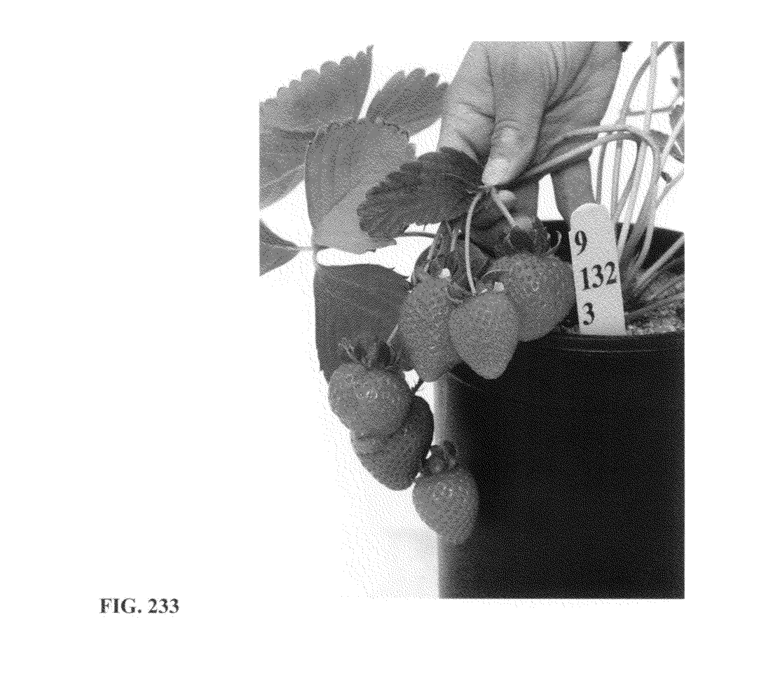

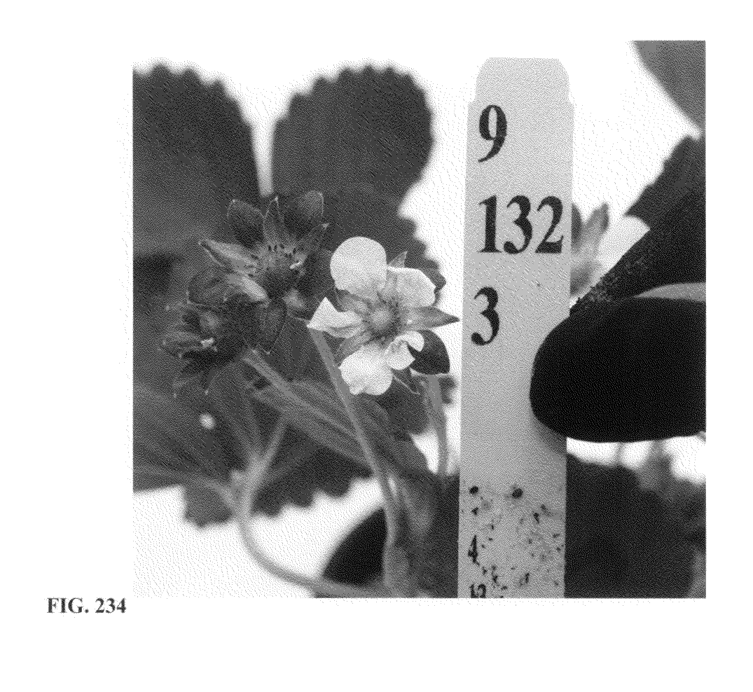

Cultivar ‘09.132-3’ has distinct disease/pathogen resistance, yield, appearance, size, and firmness. The plant of cultivar ‘09.132-3’ is characterized and distinguished in FIG. 232, the fruit of cultivar ‘09.132-3’ is characterized and distinguished in FIG. 233, and the flower of cultivar ‘09.132-3’ is characterized and distinguished in FIG. 234.

The following detailed description of cultivar ‘09.132-3’ is based on observations taken of plants and fruits (see FIGS. 232 and 233).

Certain characteristics of cultivar ‘09.132-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 78 provides data that characterizes the disease/pathogen resistance, yield, appearance, size, and firmness of cultivar ‘09.132-3’.

TABLE 78

| Cultivar | Vert | Phy | Fus | Mac | Yield | App | Size | Firm |
|---|---|---|---|---|---|---|---|---|
| 09.132-3 | 4.3 | 4.7 | 5.0 | 3.4 | 2200 | 4.1 | 31.4 | 10.7 |

### Cultivar ‘09.141-1’

In certain aspects, the present invention relates to a new and distinctive day-neutral type strawberry cultivar designated as ‘09.141-1’. Cultivar ‘09.141-1’ is typical of day-neutral strawberry cultivars and produces fruit regardless of day length when treated appropriately in arid, subtropical climates.

The strawberry cultivar ‘09.141-1’ originated from a cross between two unreleased germplasm accessions ‘05.115-5’ and ‘05.165-1’. The cultivar has been asexually reproduced by runners. The properties of this cultivar were found to be transmissible by such asexual reproduction. The cultivar is stable and reproduces true to type in successive generations of asexual reproduction.

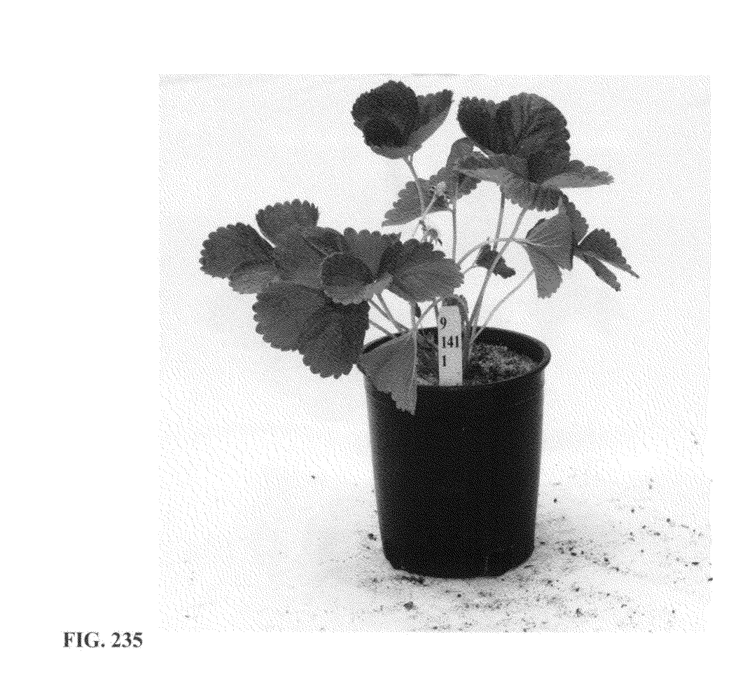

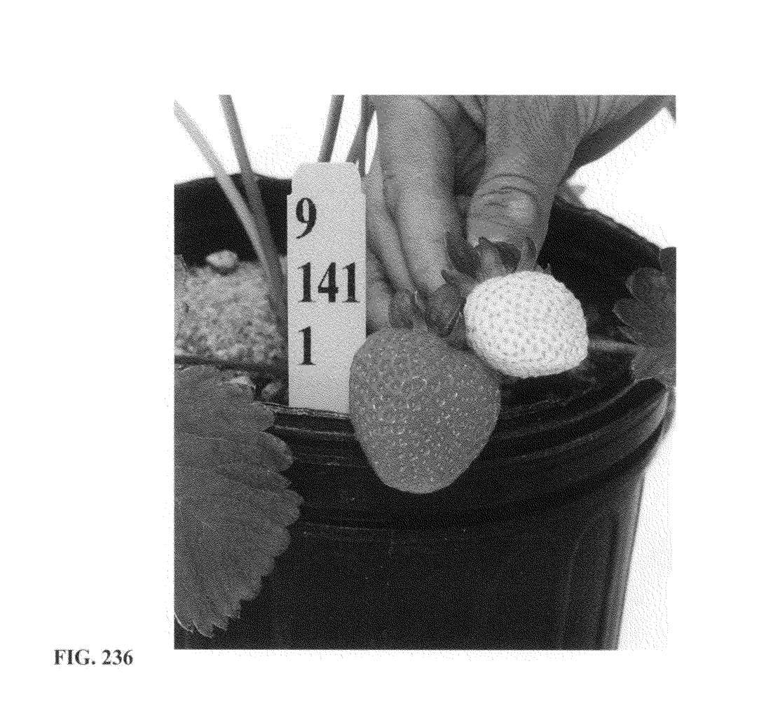

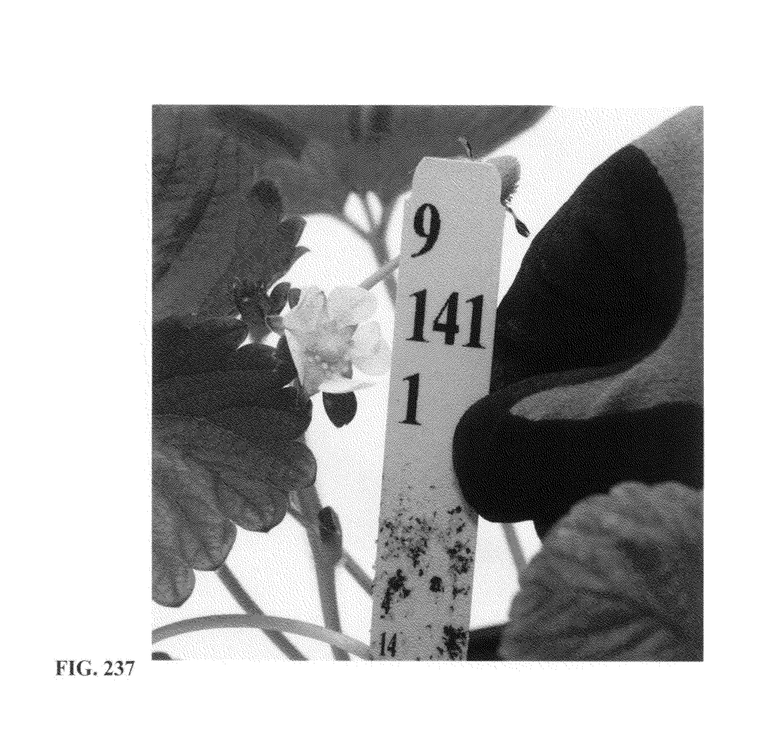

Cultivar ‘09.141-1’ has distinct disease/pathogen resistance, yield, appearance, size, and firmness. The plant of cultivar ‘09.141-1’ is characterized and distinguished in FIG. 235, the fruit of cultivar ‘09.141-1’ is characterized and distinguished in FIG. 236, and the flower of cultivar ‘09.141-1’ is characterized and distinguished in FIG. 237.

The following detailed description of cultivar ‘09.141-1’ is based on observations taken of plants and fruits (see FIGS. 235 and 236).

Certain characteristics of cultivar ‘09.141-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 79 provides data that characterizes the disease/pathogen resistance, yield, appearance, size, and firmness of cultivar ‘09.141-1’.

TABLE 79

| Cultivar | Vert | Phy | Fus | Yield | App | Size | Firm |
|---|---|---|---|---|---|---|---|
| 09.141-1 | 3.0 | 3.1 | 5.0 | 2192 | 3.8 | 30.6 | 11.7 |

### Cultivar ‘09.154-605’

In certain aspects, the present invention relates to a new and distinctive short-day type strawberry cultivar designated as ‘09.154-605’. Cultivar ‘09.154-605’ is typical of short-day strawberry cultivars and produces fruit over an extended period when treated appropriately in arid, subtropical climates.

The strawberry cultivar ‘09.154-605’ originated from a cross between two unreleased germplasm accessions ‘05.206-5’ and ‘04.9-5’. The cultivar has been asexually reproduced by runners. The properties of this cultivar were found to be transmissible by such asexual reproduction. The cultivar is stable and reproduces true to type in successive generations of asexual reproduction.

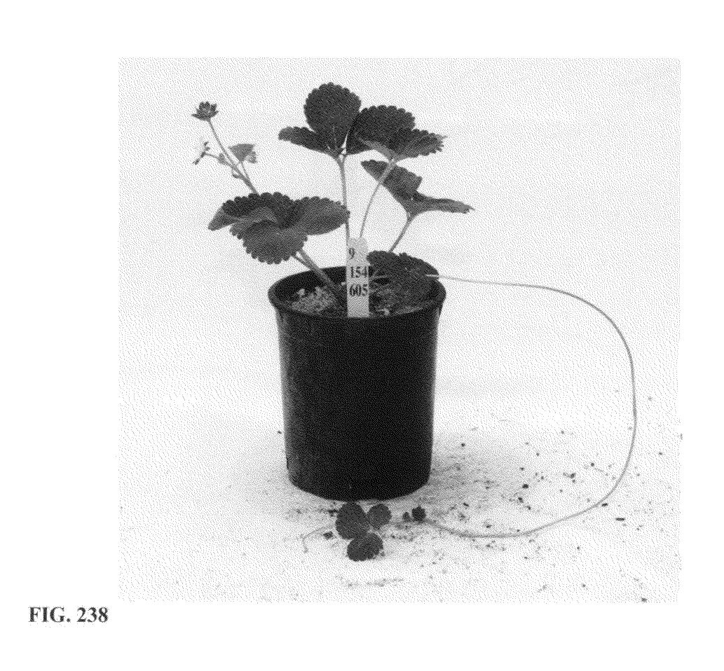

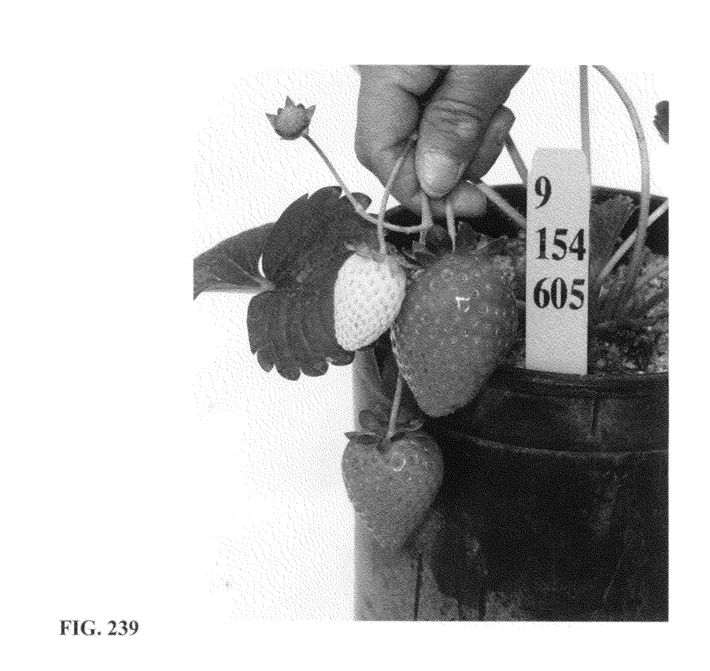

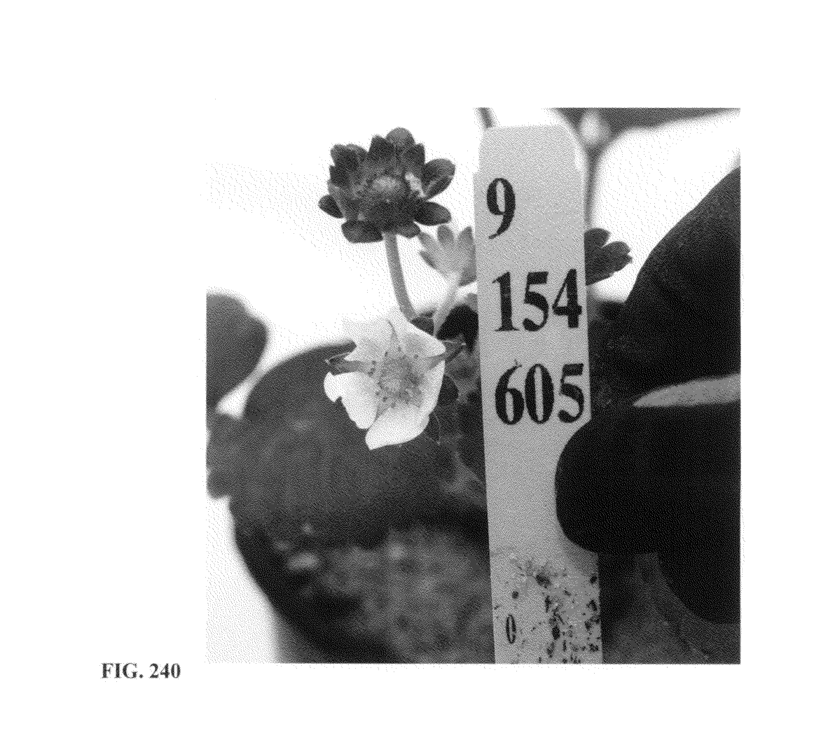

Cultivar ‘09.154-605’ has distinct disease/pathogen resistance, yield, appearance, size, and firmness. The plant of cultivar ‘09.154-605’ is characterized and distinguished in FIG. 238, the fruit of cultivar ‘09.154-605’ is characterized and distinguished in FIG. 239, and the flower of cultivar ‘09.154-605’ is characterized and distinguished in FIG. 240.

The following detailed description of cultivar ‘09.154-605’ is based on observations taken of plants and fruits (see FIGS. 238 and 239).

Certain characteristics of cultivar ‘09.154-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 80 provides data that characterizes the yield, appearance, size, and firmness of cultivar ‘09.154-605’.

TABLE 80

| Cultivar | Yield | App | Size | Firm |
|---|---|---|---|---|
| 09.154-605 | 1155 | 3.5 | 39.7 | 10.9 |

### Cultivar ‘09.166-7’

In certain aspects, the present invention relates to a new and distinctive day-neutral type strawberry cultivar designated as ‘09.166-7’. Cultivar ‘09.166-7’ is typical of day-neutral strawberry cultivars and produces fruit regardless of day length when treated appropriately in arid, subtropical climates.

The strawberry cultivar ‘09.166-7’ originated from a cross between two unreleased germplasm accessions ‘05.206-5’ and ‘05.132-1’. The cultivar has been asexually reproduced by runners. The properties of this cultivar were found to be transmissible by such asexual reproduction. The cultivar is stable and reproduces true to type in successive generations of asexual reproduction.

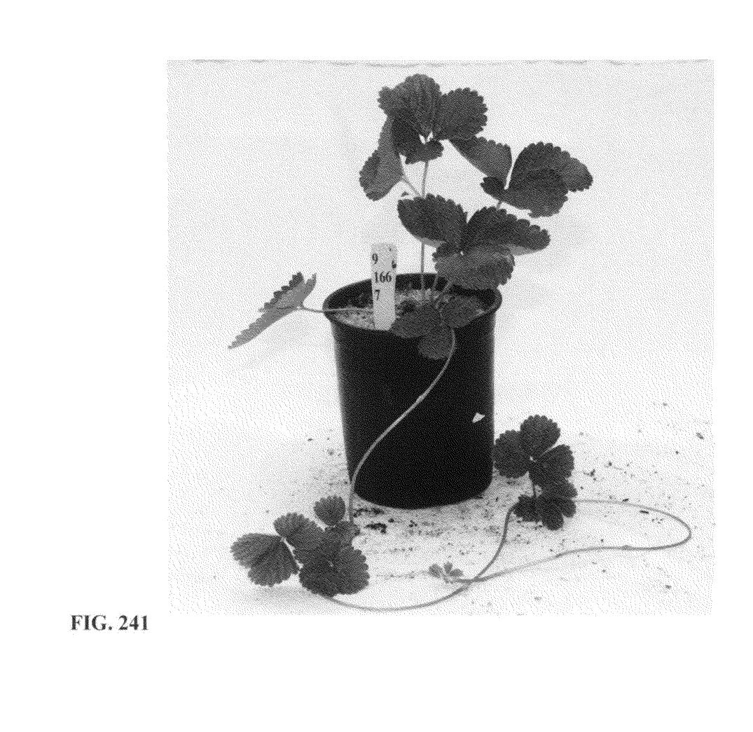

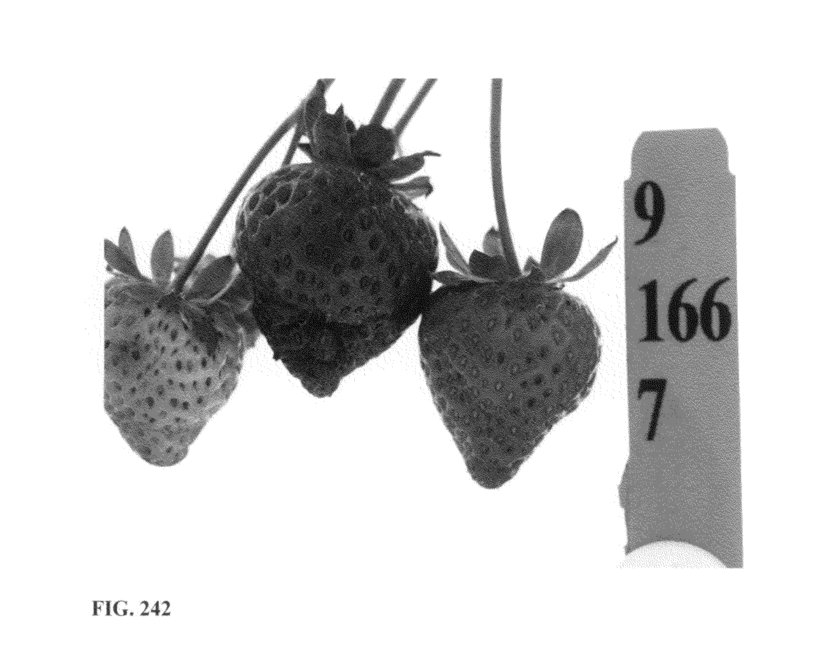

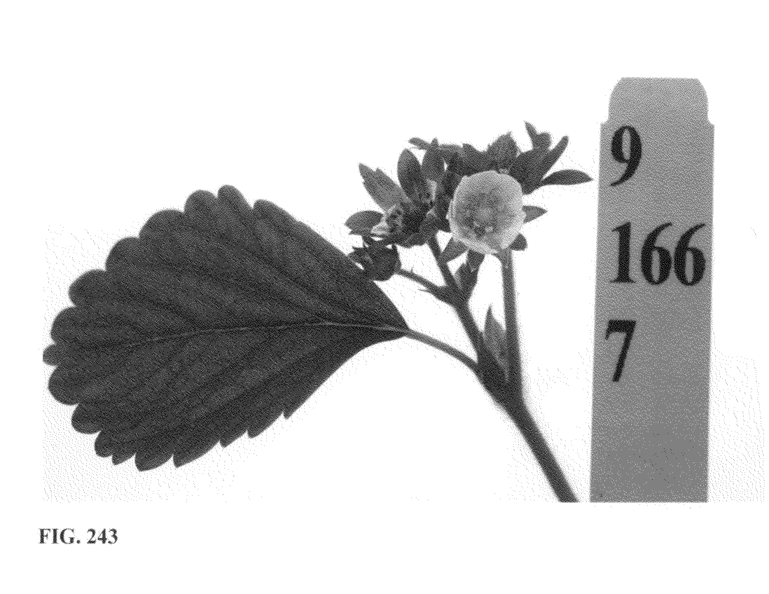

Cultivar ‘09.166-7’ has distinct disease/pathogen resistance, yield, appearance, size, and firmness. The plant of cultivar ‘09.166-7’ is characterized and distinguished in FIG. 241, the fruit of cultivar ‘09.166-7’ is characterized and distinguished in FIG. 242, and the flower of cultivar ‘09.166-7’ is characterized and distinguished in FIG. 243.

The following detailed description of cultivar ‘09.166-7’ is based on observations taken of plants and fruits (see FIGS. 241 and 242).

Certain characteristics of cultivar ‘09.166-7’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 81 provides data that characterizes the disease/pathogen resistance, yield, appearance, size, and firmness of cultivar ‘09.166-7’.

TABLE 81

| Cultivar | Vert | Fus | Yield | App | Size | Firm |
|---|---|---|---|---|---|---|
| 09.166-7 | 3.7 | 5.0 | 2401 | 3.7 | 31.7 | 11.9 |

### Cultivar ‘09.171-602’

In certain aspects, the present invention relates to a new and distinctive short-day type strawberry cultivar designated as ‘09.171-602’. Cultivar ‘09.171-602’ is typical of short-day strawberry cultivars and produces fruit over an extended period when treated appropriately in arid, subtropical climates.

The strawberry cultivar ‘09.171-602’ originated from a cross between two unreleased germplasm accessions ‘05.194-1’ and ‘05.148-2’. The cultivar has been asexually reproduced by runners. The properties of this cultivar were found to be transmissible by such asexual reproduction. The cultivar is stable and reproduces true to type in successive generations of asexual reproduction.

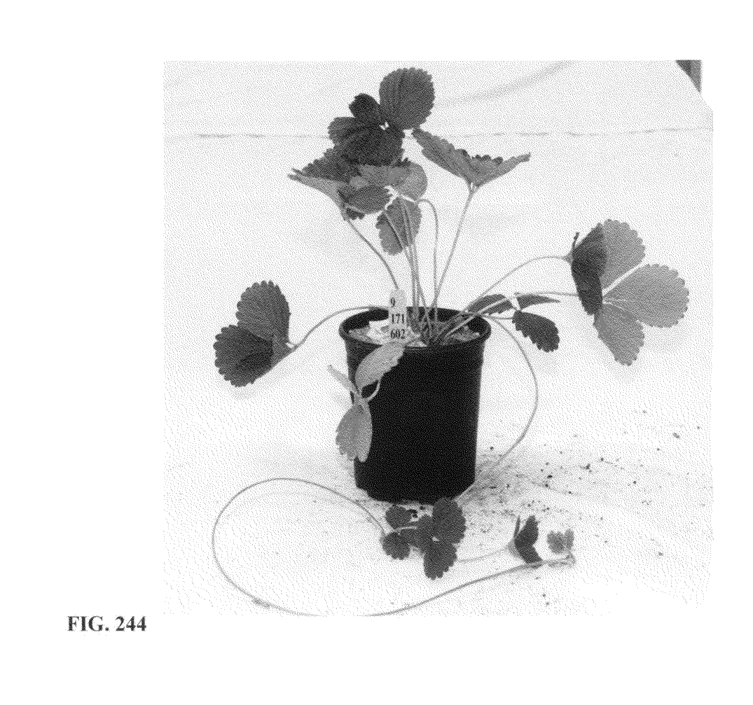

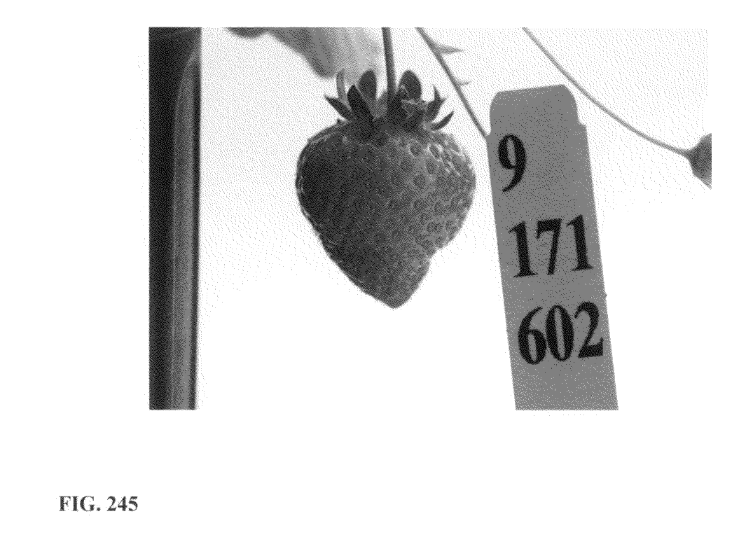

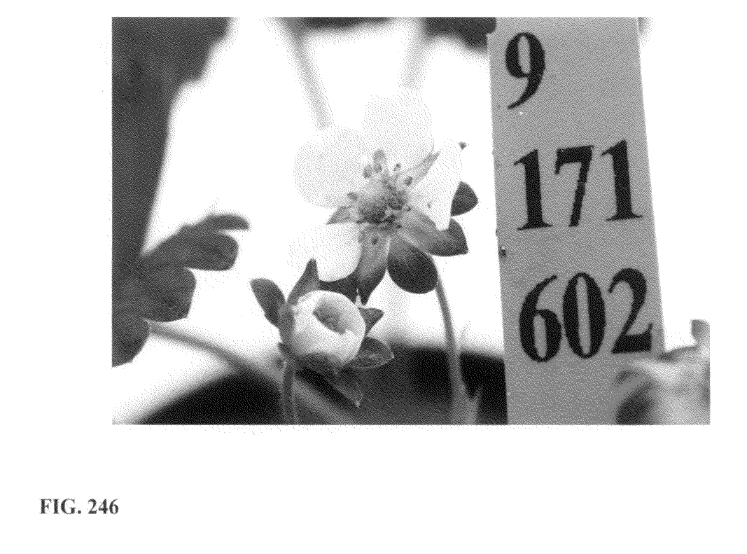

Cultivar ‘09.171-602’ has distinct disease/pathogen resistance, yield, appearance, size, and firmness. The plant of cultivar ‘09.171-602’ is characterized and distinguished in FIG. 244, the fruit of cultivar ‘09.171-602’ is characterized and distinguished in FIG. 245, and the flower of cultivar ‘09.171-602’ is characterized and distinguished in FIG. 246.

The following detailed description of cultivar ‘09.171-602’ is based on observations taken of plants and fruits (see FIGS. 244 and 245).

Certain characteristics of cultivar ‘09.171-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 82 provides data that characterizes the yield, appearance, size, and firmness of cultivar ‘09.171-602’.

TABLE 82

| Cultivar | Yield | App | Size | Firm |
|---|---|---|---|---|
| 09.171-602 | 2552 | 3.8 | 33.2 | 11.7 |

### Cultivar ‘09.180-1’

In certain aspects, the present invention relates to a new and distinctive day-neutral type strawberry cultivar designated as ‘09.180-1’. Cultivar ‘09.180-1’ is typical of day-neutral strawberry cultivars and produces fruit regardless of day length when treated appropriately in arid, subtropical climates.

The strawberry cultivar ‘09.180-1’ originated from a cross between two unreleased germplasm accessions ‘06.171-5’ and ‘05.205-2’. The cultivar has been asexually reproduced by runners. The properties of this cultivar were found to be transmissible by such asexual reproduction. The cultivar is stable and reproduces true to type in successive generations of asexual reproduction.

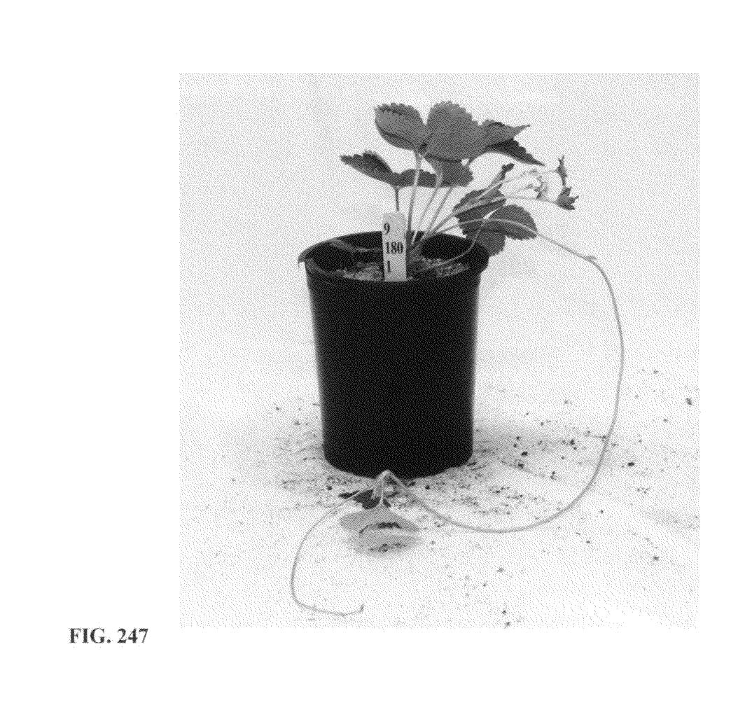

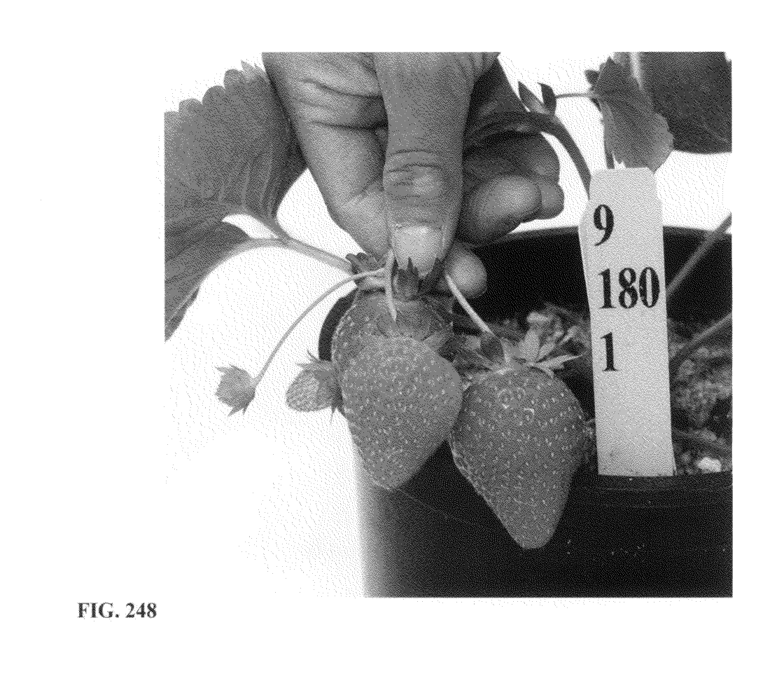

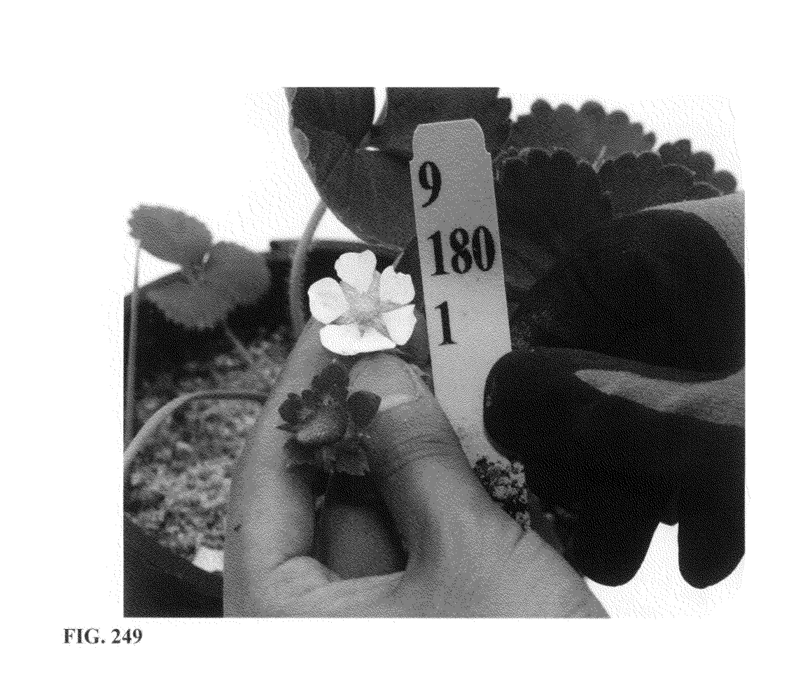

Cultivar ‘09.180-1’ has distinct disease/pathogen resistance, yield, appearance, size, and firmness. The plant of cultivar ‘09.180-1’ is characterized and distinguished in FIG. 247, the fruit of cultivar ‘09.180-1’ is characterized and distinguished in FIG. 248, and the flower of cultivar ‘09.180-1’ is characterized and distinguished in FIG. 249.

The following detailed description of cultivar ‘09.180-1’ is based on observations taken of plants and fruits (see FIGS. 247 and 248).

Certain characteristics of cultivar ‘09.180-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 83 provides data that characterizes the disease/pathogen resistance, yield, appearance, size, and firmness of cultivar ‘09.180-1’.

TABLE 83

| Cultivar | Phy | Fus | Mac | Yield | App | Size | Firm |
|---|---|---|---|---|---|---|---|
| 09.180-1 | 2.4 | 4.8 | 3.8 | 2626 | 4.4 | 36.1 | 9.9 |

### Cultivar ‘10.1-604’

In certain aspects, the present invention relates to a new and distinctive short-day type strawberry cultivar designated as ‘10.1-604’. Cultivar ‘10.1-604’ is typical of short-day strawberry cultivars and produces fruit over an extended period when treated appropriately in arid, subtropical climates.

The strawberry cultivar ‘10.1-604’ originated from a cross between two unreleased germplasm accessions ‘05.54-611’ and ‘06.20-605’. The cultivar has been asexually reproduced by runners. The properties of this cultivar were found to be transmissible by such asexual reproduction. The cultivar is stable and reproduces true to type in successive generations of asexual reproduction.

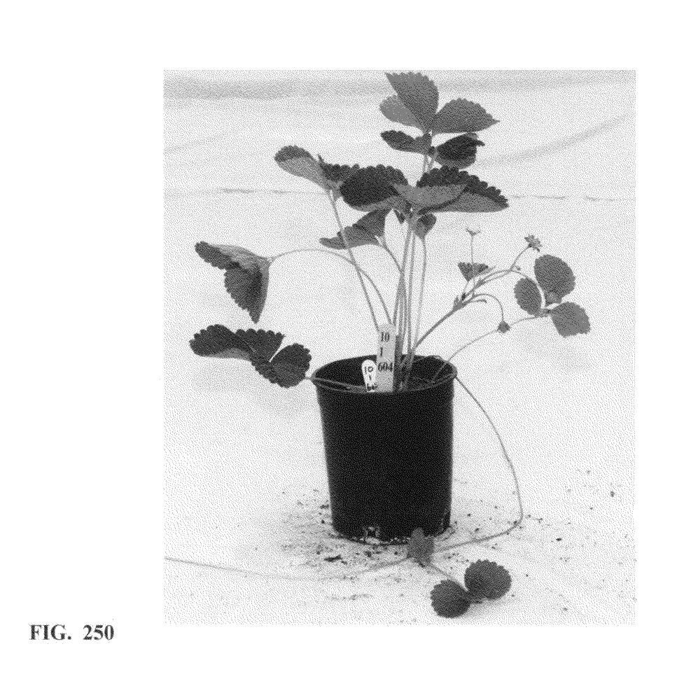

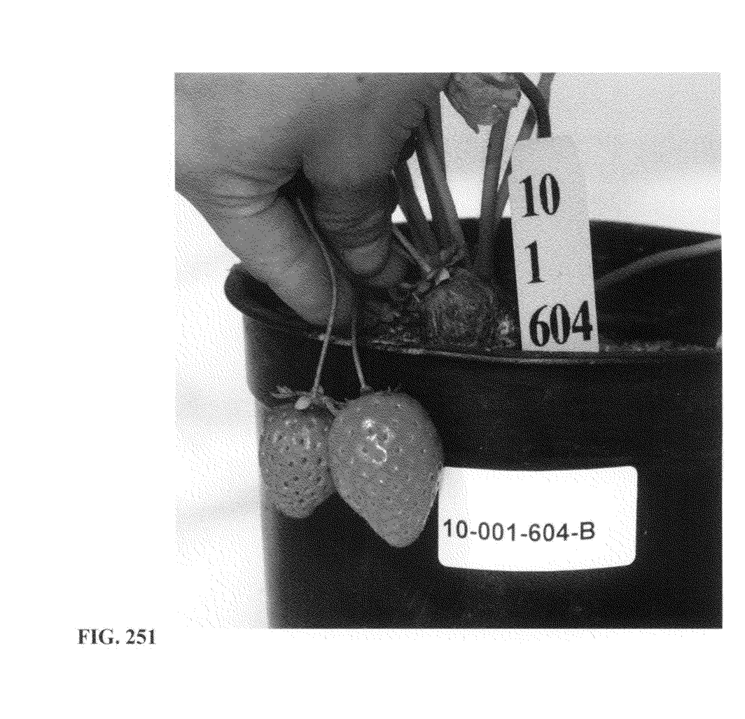

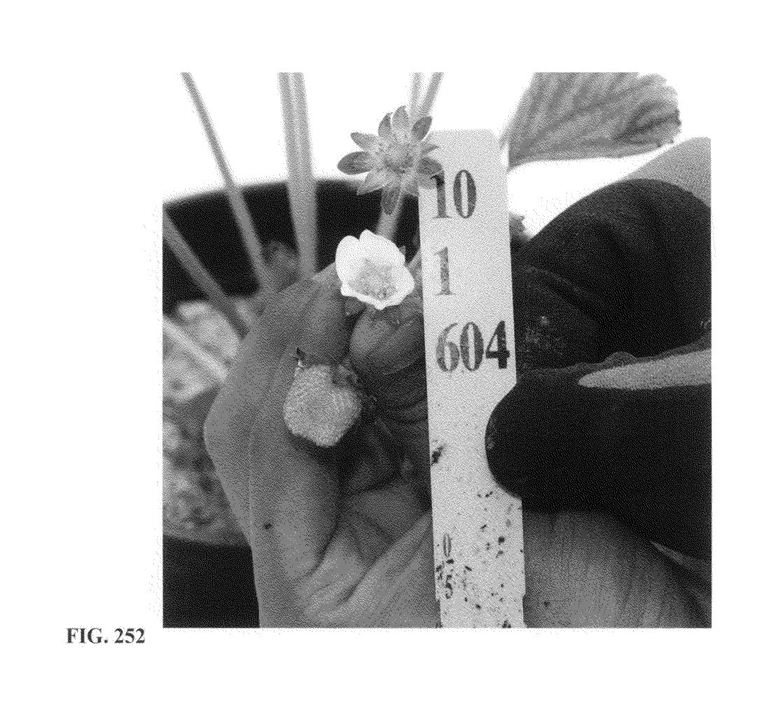

Cultivar ‘10.1-604’ has distinct disease/pathogen resistance, yield, appearance, size, and firmness. The plant of cultivar ‘10.1-604’ is characterized and distinguished in FIG. 250, the fruit of cultivar ‘10.1-604’ is characterized and distinguished in FIG. 251, and the flower of cultivar ‘10.1-604’ is characterized and distinguished in FIG. 252.

The following detailed description of cultivar ‘10.1-604’ is based on observations taken of plants and fruits (see FIGS. 250 and 251).

Certain characteristics of cultivar ‘10.1-604’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 84 provides data that characterizes the yield, appearance, size, and firmness of cultivar ‘10.1-604’.

TABLE 84

| Cultivar | Yield | App | Size | Firm |
|---|---|---|---|---|
| 10.1-604 | 1455 | 3.7 | 41.4 | 10.9 |

### Cultivar ‘10.5-602’

In certain aspects, the present invention relates to a new and distinctive short-day type strawberry cultivar designated as ‘10.5-602’. Cultivar ‘10.5-602’ is typical of short-day strawberry cultivars and produces fruit over an extended period when treated appropriately in arid, subtropical climates.

The strawberry cultivar ‘10.5-602’ originated from a cross between two unreleased germplasm accessions ‘06.137-2’ and ‘07.64-3’. The cultivar has been asexually reproduced by runners. The properties of this cultivar were found to be transmissible by such asexual reproduction. The cultivar is stable and reproduces true to type in successive generations of asexual reproduction.

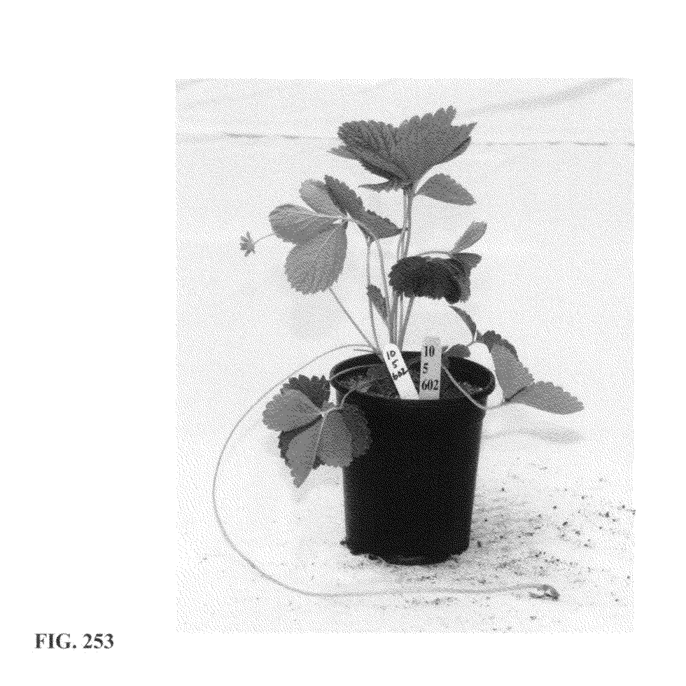

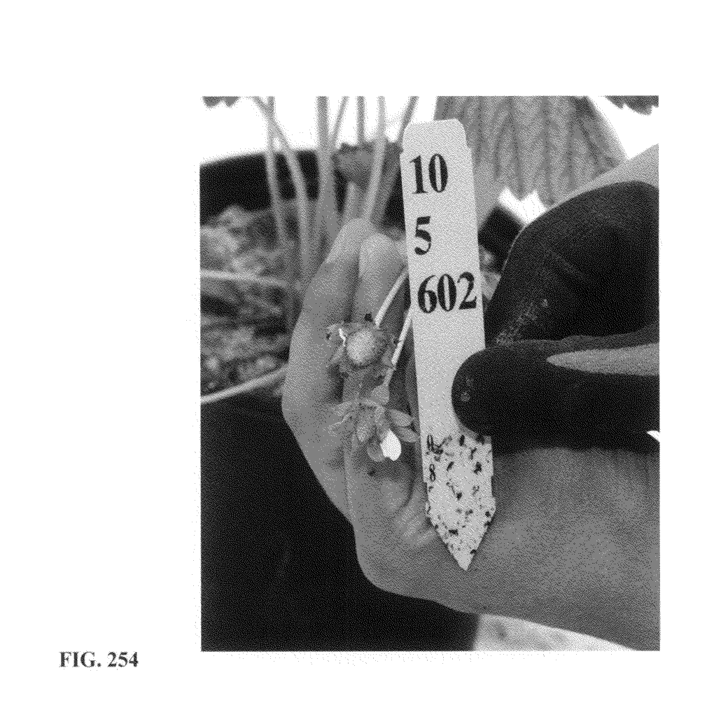

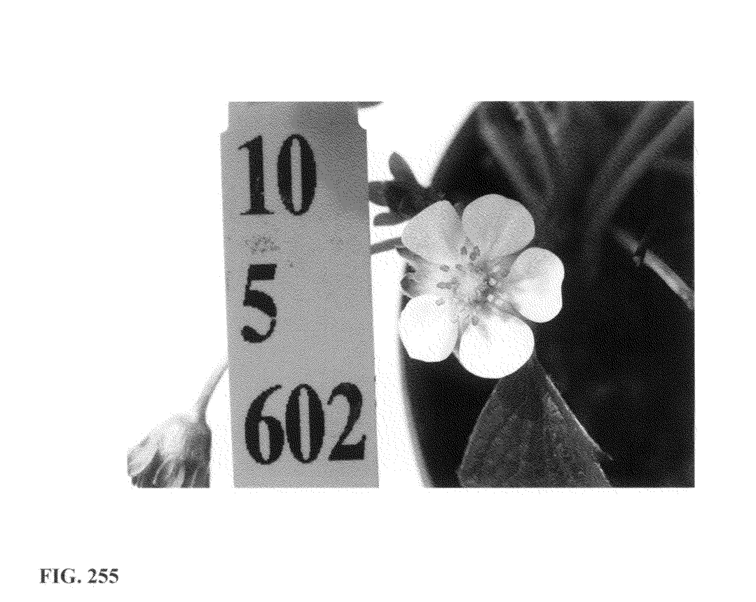

Cultivar ‘10.5-602’ has distinct disease/pathogen resistance, yield, appearance, size, and firmness. The plant of cultivar ‘10.5-602’ is characterized and distinguished in FIG. 253, the fruit of cultivar ‘10.5-602’ is characterized and distinguished in FIG. 254, and the flower of cultivar ‘10.5-602’ is characterized and distinguished in FIG. 255.

The following detailed description of cultivar ‘10.5-602’ is based on observations taken of plants and fruits (see FIGS. 253 and 254).

Certain characteristics of cultivar ‘10.5-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 85 provides data that characterizes the yield, appearance, size, and firmness of cultivar ‘10.5-602’.

TABLE 85

| Cultivar | Yield | App | Size | Firm |
|---|---|---|---|---|
| 10.5-602 | 1256 | 3.9 | 41.7 | 10.9 |

### Cultivar ‘10.7-3’

In certain aspects, the present invention relates to a new and distinctive short-day type strawberry cultivar designated as ‘10.7-3’. Cultivar ‘10.7-3’ is typical of short-day strawberry cultivars and produces fruit over an extended period when treated appropriately in arid, subtropical climates.

The strawberry cultivar ‘10.7-3’ originated from a cross between two unreleased germplasm accessions ‘05.54-611’ and ‘07.64-3’. The cultivar has been asexually reproduced by runners. The properties of this cultivar were found to be transmissible by such asexual reproduction. The cultivar is stable and reproduces true to type in successive generations of asexual reproduction.

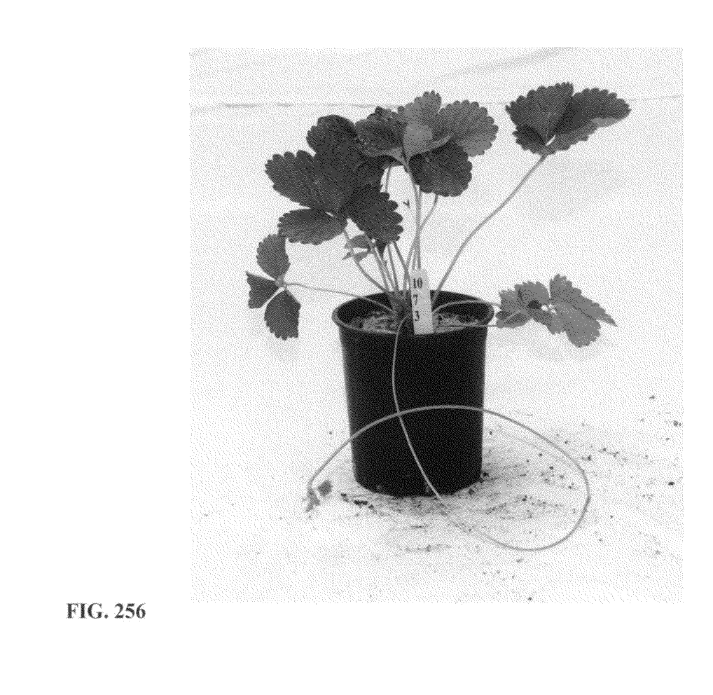

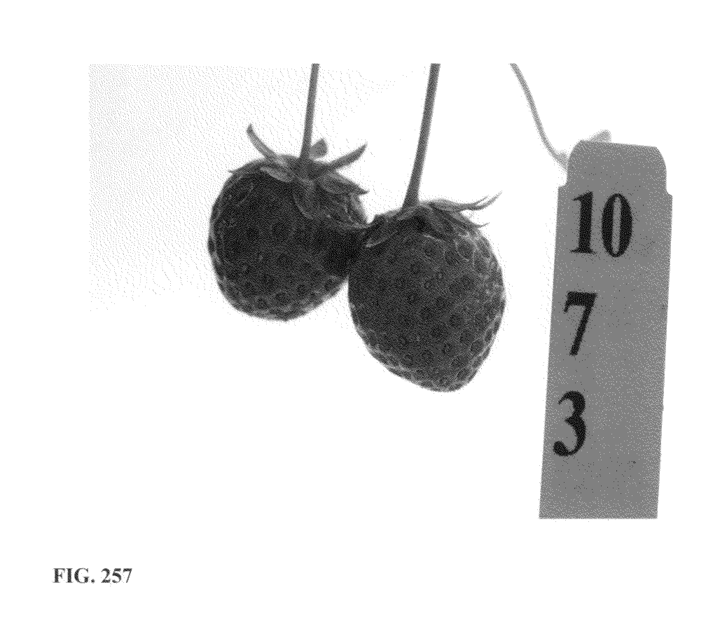

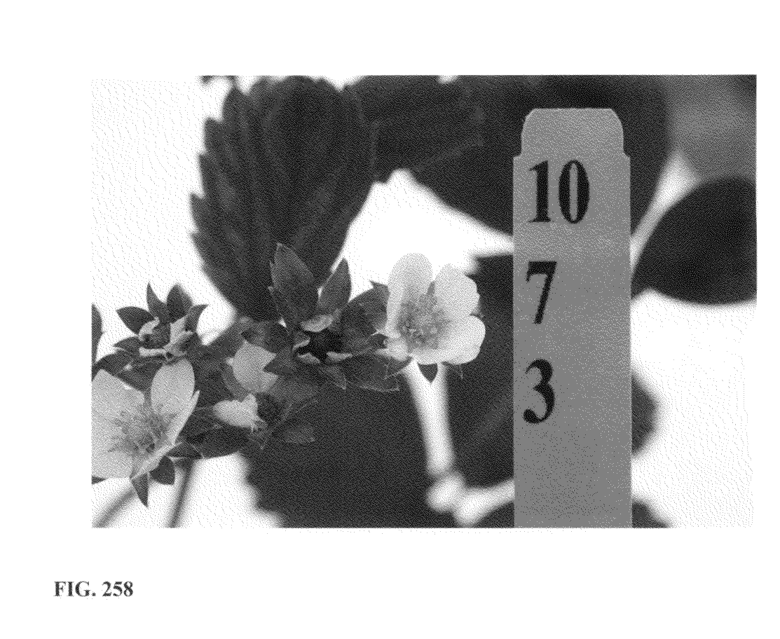

Cultivar ‘10.7-3’ has distinct disease/pathogen resistance, yield, appearance, size, and firmness. The plant of cultivar ‘10.7-3’ is characterized and distinguished in FIG. 256, the fruit of cultivar ‘10.7-3’ is characterized and distinguished in FIG. 257, and the flower of cultivar ‘10.7-3’ is characterized and distinguished in FIG. 258.

The following detailed description of cultivar ‘10.7-3’ is based on observations taken of plants and fruits (see FIGS. 256 and 257).

Certain characteristics of cultivar ‘10.7-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 86 provides data that characterizes the yield, appearance, size, and firmness of cultivar ‘10.7-3’.

TABLE 86

| Cultivar | Yield | App | Size | Firm |
|---|---|---|---|---|
| 10.7-3 | 1934 | 3.8 | 37.3 | 11.3 |

### Cultivar ‘10.12-3’

In certain aspects, the present invention relates to a new and distinctive short-day type strawberry cultivar designated as ‘10.12-3’. Cultivar ‘10.12-3’ is typical of short-day strawberry cultivars and produces fruit over an extended period when treated appropriately in arid, subtropical climates.

The strawberry cultivar ‘10.12-3’ originated from a cross between unreleased germplasm accession ‘06.20-605’ and cultivar ‘Merced’. The cultivar has been asexually reproduced by runners. The properties of this cultivar were found to be transmissible by such asexual reproduction. The cultivar is stable and reproduces true to type in successive generations of asexual reproduction.

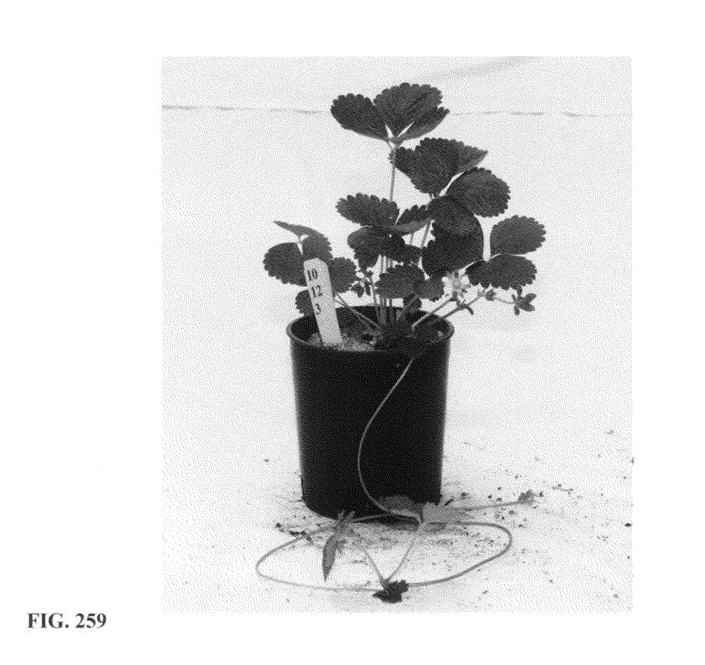

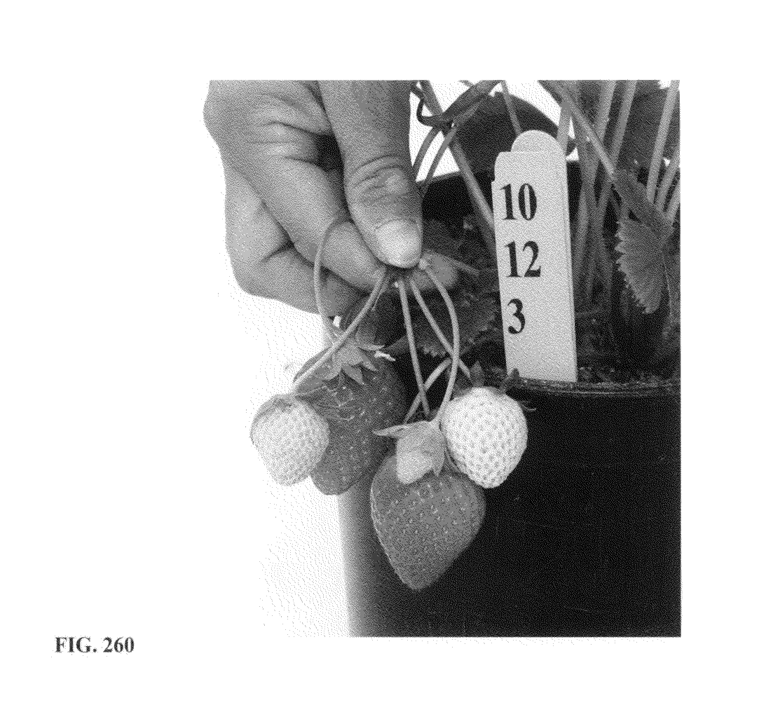

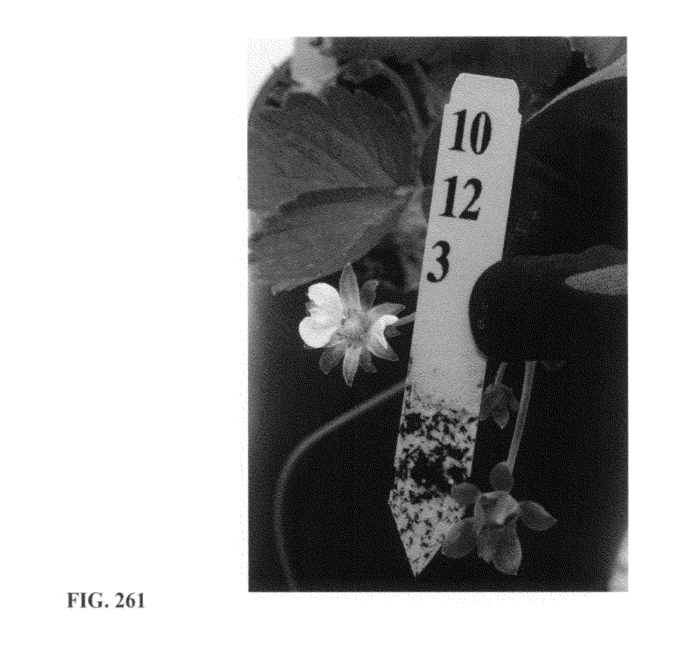

Cultivar ‘10.12-3’ has distinct disease/pathogen resistance, yield, appearance, size, and firmness. The plant of cultivar ‘10.12-3’ is characterized and distinguished in FIG. 259, the fruit of cultivar ‘10.12-3’ is characterized and distinguished in FIG. 260, and the flower of cultivar ‘10.12-3’ is characterized and distinguished in FIG. 261.

The following detailed description of cultivar ‘10.12-3’ is based on observations taken of plants and fruits (see FIGS. 259 and 260).

Certain characteristics of cultivar ‘10.12-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 87 provides data that characterizes the yield, appearance, size, and firmness of cultivar ‘10.12-3’.

TABLE 87

| Cultivar | Yield | App | Size | Firm |
|---|---|---|---|---|
| 10.12-3 | 2080 | 3.5 | 30.7 | 11.8 |

### Cultivar ‘10.15-605’

In certain aspects, the present invention relates to a new and distinctive short-day type strawberry cultivar designated as ‘10.15-605’. Cultivar ‘10.15-605’ is typical of short-day strawberry cultivars and produces fruit over an extended period when treated appropriately in arid, subtropical climates.

The strawberry cultivar ‘10.15-605’ originated from a cross between unreleased germplasm accession ‘07.64-3’ and cultivar ‘Merced’. The cultivar has been asexually reproduced by runners. The properties of this cultivar were found to be transmissible by such asexual reproduction. The cultivar is stable and reproduces true to type in successive generations of asexual reproduction.

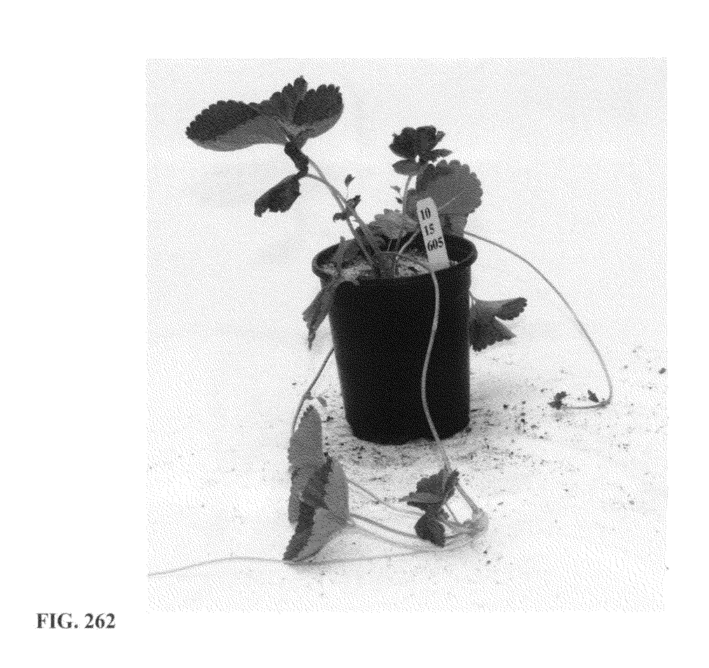

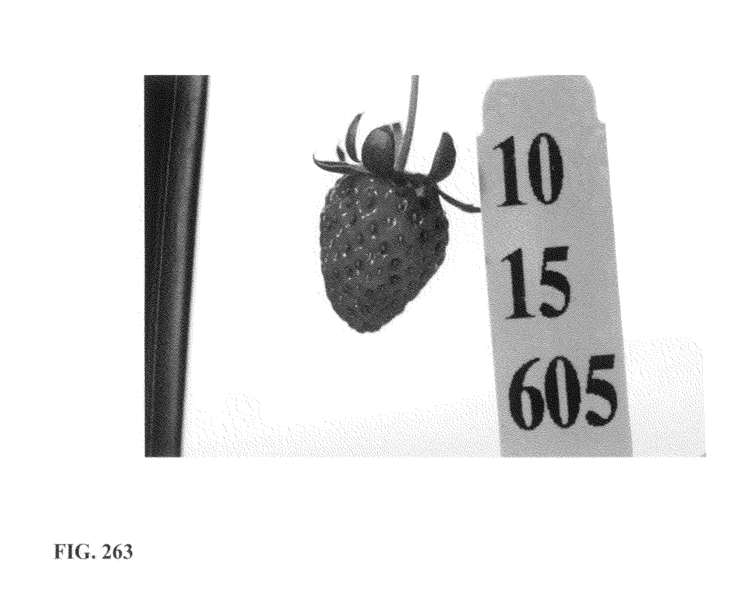

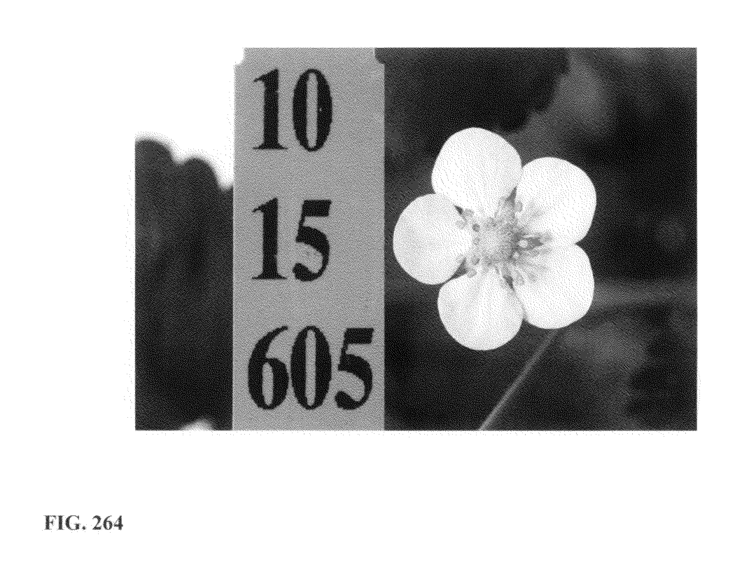

Cultivar ‘10.15-605’ has distinct disease/pathogen resistance, yield, appearance, size, and firmness. The plant of cultivar ‘10.15-605’ is characterized and distinguished in FIG. 262, the fruit of cultivar ‘10.15-605’ is characterized and distinguished in FIG. 263, and the flower of cultivar ‘10.15-605’ is characterized and distinguished in FIG. 264.

The following detailed description of cultivar ‘10.15-605’ is based on observations taken of plants and fruits (see FIGS. 262 and 263).

Certain characteristics of cultivar ‘10.15-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 88 provides data that characterizes the yield, appearance, size, and firmness of cultivar ‘10.15-605’.

TABLE 88

| Cultivar | Yield | App | Size | Firm |
|---|---|---|---|---|
| 10.15-605 | 1101 | 3.4 | 37.7 | 12.0 |

### Cultivar ‘10.17-1’

In certain aspects, the present invention relates to a new and distinctive short-day type strawberry cultivar designated as ‘10.17-1’. Cultivar ‘10.17-1’ is typical of short-day strawberry cultivars and produces fruit over an extended period when treated appropriately in arid, subtropical climates.

The strawberry cultivar ‘10.17-1’ originated from a cross between two unreleased germplasm accessions ‘06.20-605’ and ‘07.134-1’. The cultivar has been asexually reproduced by runners. The properties of this cultivar were found to be transmissible by such asexual reproduction. The cultivar is stable and reproduces true to type in successive generations of asexual reproduction.

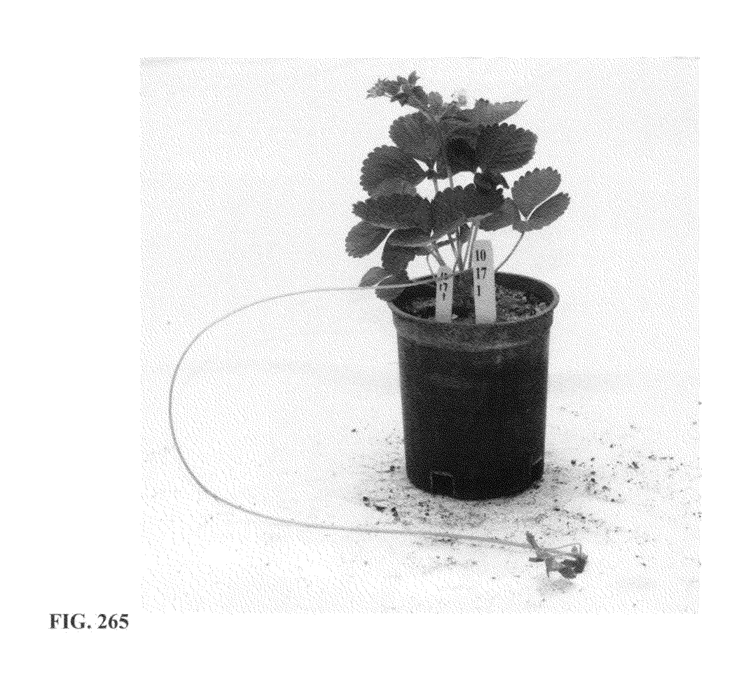

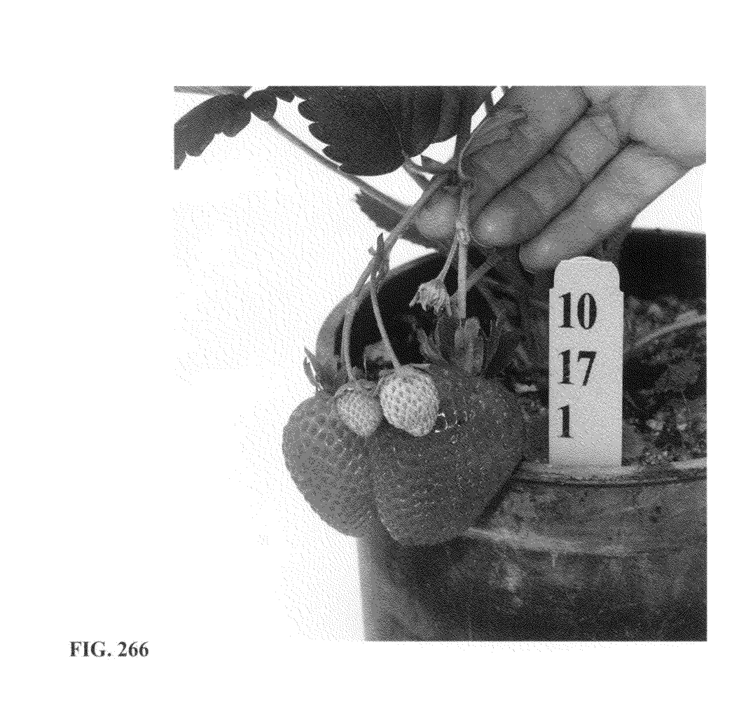

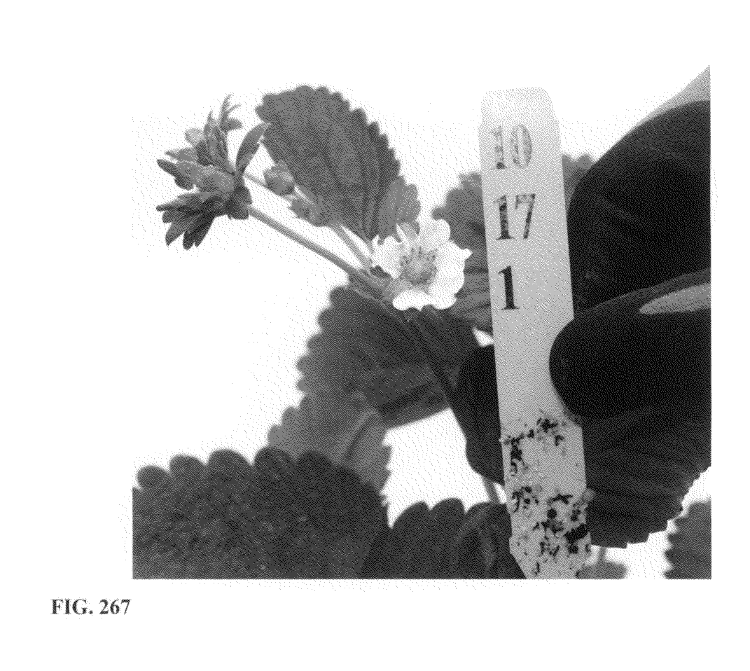

Cultivar ‘10.17-1’ has distinct disease/pathogen resistance, yield, appearance, size, and firmness. The plant of cultivar ‘10.17-1’ is characterized and distinguished in FIG. 265, the fruit of cultivar ‘10.17-1’ is characterized and distinguished in FIG. 266, and the flower of cultivar ‘10.17-1’ is characterized and distinguished in FIG. 267.

The following detailed description of cultivar ‘10.17-1’ is based on observations taken of plants and fruits (see FIGS. 265 and 266).

Certain characteristics of cultivar ‘10.17-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 89 provides data that characterizes the yield, appearance, size, and firmness of cultivar ‘10.17-1’.

TABLE 89

| Cultivar | Yield | App | Size | Firm |
|---|---|---|---|---|
| 10.17-1 | 2059 | 3.7 | 32.6 | 12.7 |

### Cultivar ‘10.17-2’

In certain aspects, the present invention relates to a new and distinctive short-day type strawberry cultivar designated as ‘10.17-2’. Cultivar ‘10.17-2’ is typical of short-day strawberry cultivars and produces fruit over an extended period when treated appropriately in arid, subtropical climates.

The strawberry cultivar ‘10.17-2’ originated from a cross between two unreleased germplasm accessions ‘06.20-605’ and ‘07.134-1’. The cultivar has been asexually reproduced by runners. The properties of this cultivar were found to be transmissible by such asexual reproduction. The cultivar is stable and reproduces true to type in successive generations of asexual reproduction.

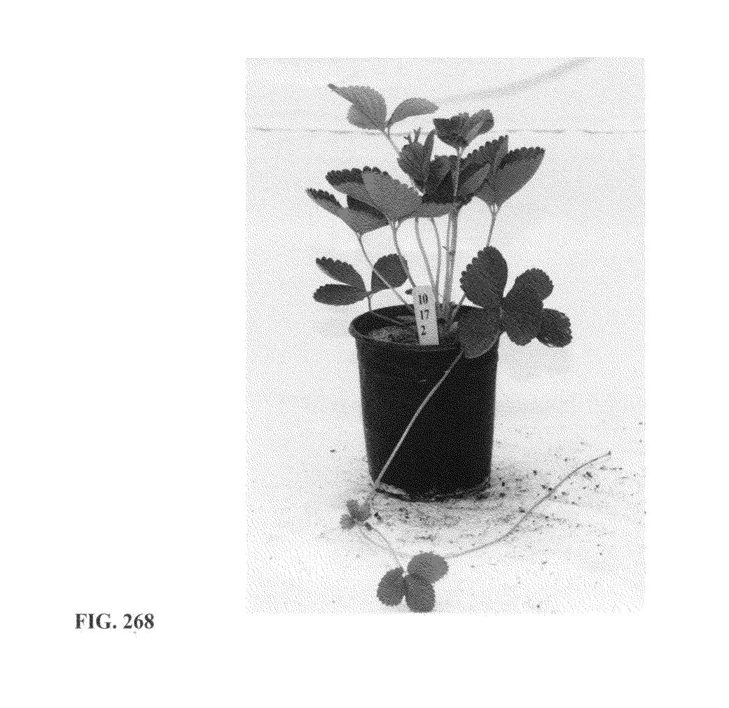

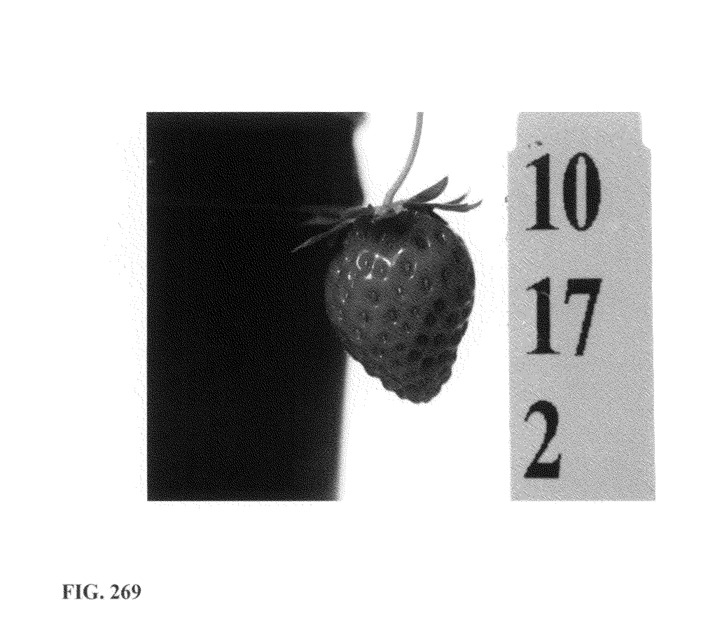

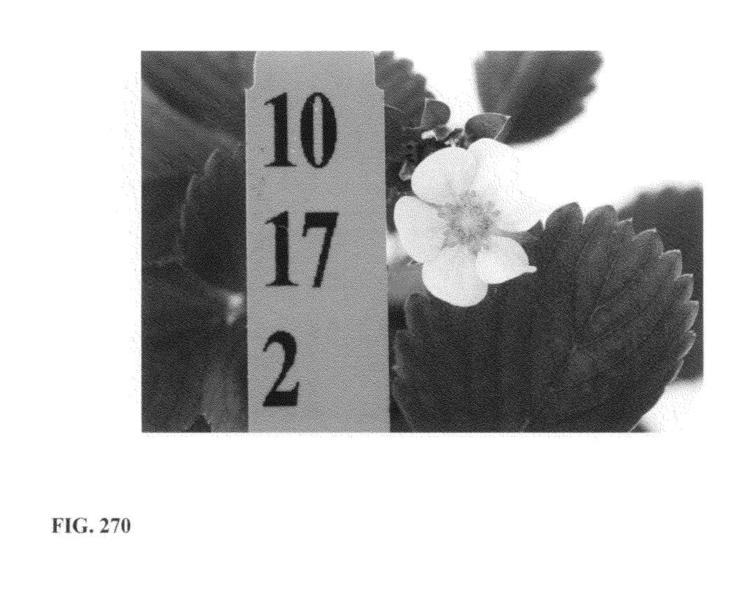

Cultivar ‘10.17-2’ has distinct disease/pathogen resistance, yield, appearance, size, and firmness. The plant of cultivar ‘10.17-2’ is characterized and distinguished in FIG. 268, the fruit of cultivar ‘10.17-2’ is characterized and distinguished in FIG. 269, and the flower of cultivar ‘10.17-2’ is characterized and distinguished in FIG. 270.

The following detailed description of cultivar ‘10.17-2’ is based on observations taken of plants and fruits (see FIGS. 268 and 269).

Certain characteristics of cultivar ‘10.17-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 90 provides data that characterizes the yield, appearance, size, and firmness of cultivar ‘10.17-2’.

TABLE 90

| Cultivar | Yield | App | Size | Firm |
|---|---|---|---|---|
| 10.17-2 | 2247 | 3.8 | 32.5 | 11.6 |

### Cultivar ‘10.23-1’

In certain aspects, the present invention relates to a new and distinctive short-day type strawberry cultivar designated as ‘10.23-1’. Cultivar ‘10.23-1’ is typical of short-day strawberry cultivars and produces fruit over an extended period when treated appropriately in arid, subtropical climates.

The strawberry cultivar ‘10.23-1’ originated from a cross between two unreleased germplasm accessions ‘05.107-2’ and ‘05.27-605’. The cultivar has been asexually reproduced by runners. The properties of this cultivar were found to be transmissible by such asexual reproduction. The cultivar is stable and reproduces true to type in successive generations of asexual reproduction.

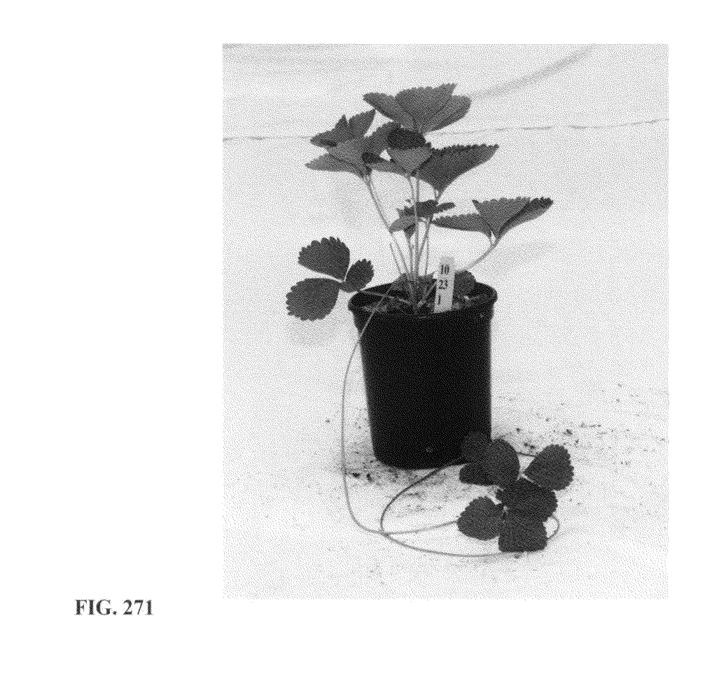

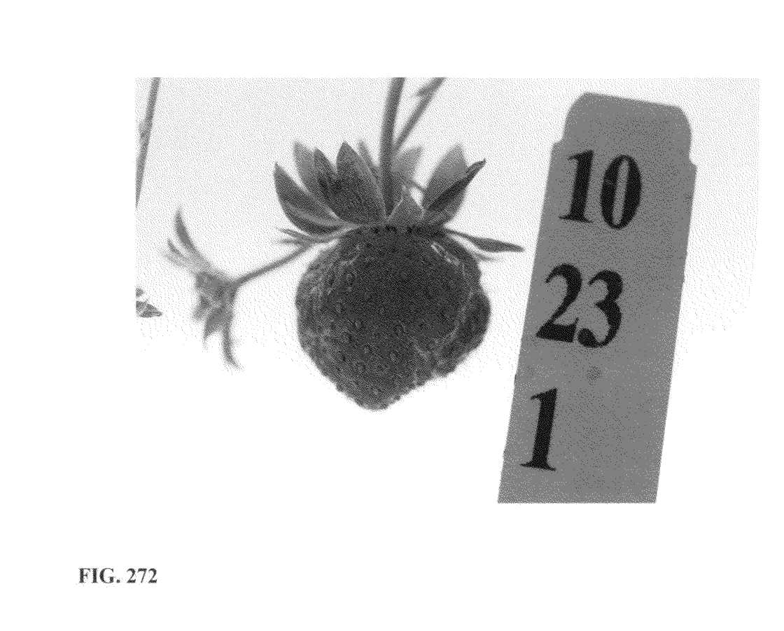

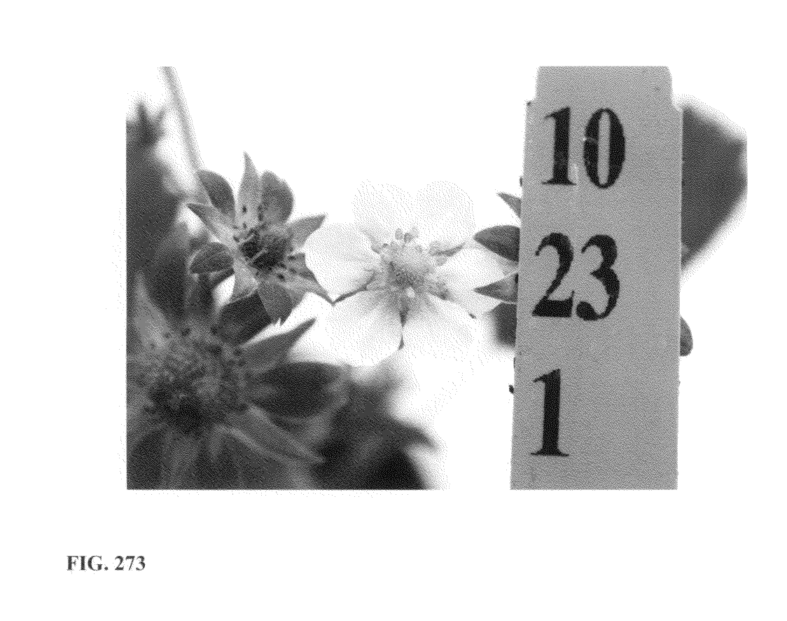

Cultivar ‘10.23-1’ has distinct disease/pathogen resistance, yield, appearance, size, and firmness. The plant of cultivar ‘10.23-1’ is characterized and distinguished in FIG. 271, the fruit of cultivar ‘10.23-1’ is characterized and distinguished in FIG. 272, and the flower of cultivar ‘10.23-1’ is characterized and distinguished in FIG. 273.

The following detailed description of cultivar ‘10.23-1’ is based on observations taken of plants and fruits (see FIGS. 271 and 272).

Certain characteristics of cultivar ‘10.23-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 91 provides data that characterizes the yield, appearance, size, and firmness of cultivar ‘10.23-1’.

TABLE 91

| Cultivar | Yield | App | Size | Firm |
|---|---|---|---|---|
| 10.23-1 | 2312 | 3.4 | 34.0 | 9.3 |

### Cultivar ‘10.24-2’

In certain aspects, the present invention relates to a new and distinctive day-neutral type strawberry cultivar designated as ‘10.24-2’. Cultivar ‘10.24-2’ is typical of day-neutral strawberry cultivars and produces fruit regardless of day length when treated appropriately in arid, subtropical climates.

The strawberry cultivar ‘10.24-2’ originated from a cross between two unreleased germplasm accessions ‘05.108-7’ and ‘05.27-605’. The cultivar has been asexually reproduced by runners. The properties of this cultivar were found to be transmissible by such asexual reproduction. The cultivar is stable and reproduces true to type in successive generations of asexual reproduction.

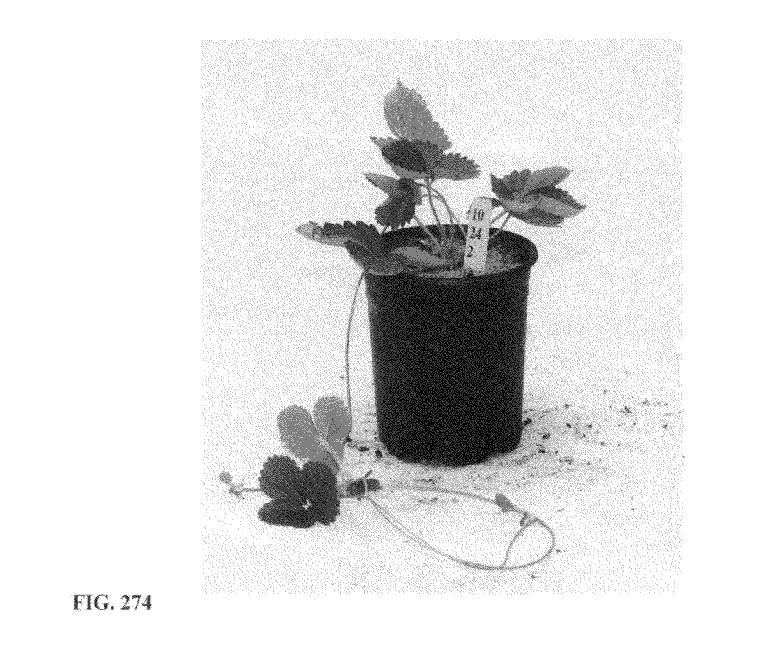

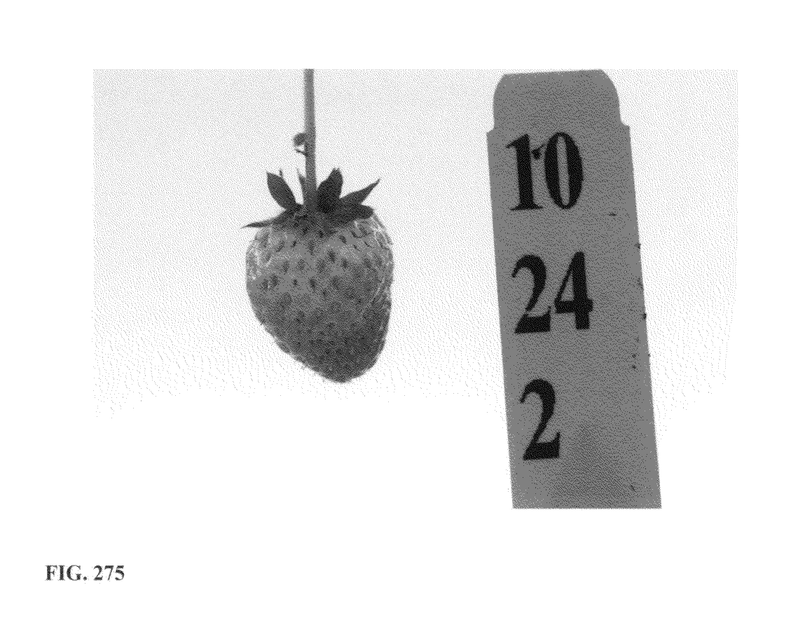

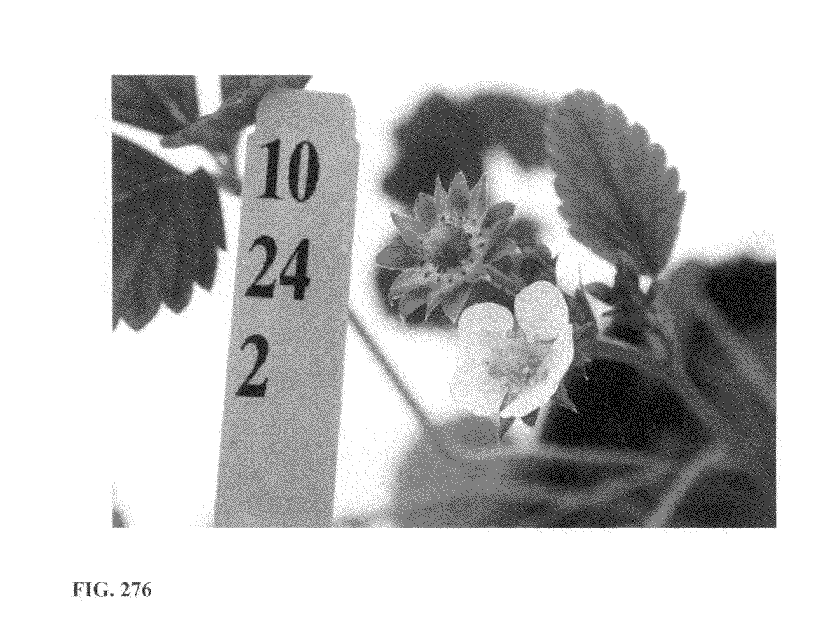

Cultivar ‘10.24-2’ has distinct disease/pathogen resistance, yield, appearance, size, and firmness. The plant of cultivar ‘10.24-2’ is characterized and distinguished in FIG. 274, the fruit of cultivar ‘10.24-2’ is characterized and distinguished in FIG. 275, and the flower of cultivar ‘10.24-2’ is characterized and distinguished in FIG. 276.

The following detailed description of cultivar ‘10.24-2’ is based on observations taken of plants and fruits (see FIGS. 274 and 275).

Certain characteristics of cultivar ‘10.24-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 92 provides data that characterizes the yield, appearance, size, and firmness of cultivar ‘10.24-2’.

TABLE 92

| Cultivar | Yield | App | Size | Firm |
|---|---|---|---|---|
| 10.24-2 | 2509 | 3.8 | 31.8 | 10.9 |

### Cultivar ‘10.25-601’

In certain aspects, the present invention relates to a new and distinctive short-day type strawberry cultivar designated as ‘10.25-601’. Cultivar ‘10.25-601’ is typical of short-day strawberry cultivars and produces fruit over an extended period when treated appropriately in arid, subtropical climates.

The strawberry cultivar ‘10.25-601’ originated from a cross between two unreleased germplasm accessions ‘06.16-602’ and ‘05.27-605’. The cultivar has been asexually reproduced by runners. The properties of this cultivar were found to be transmissible by such asexual reproduction. The cultivar is stable and reproduces true to type in successive generations of asexual reproduction.

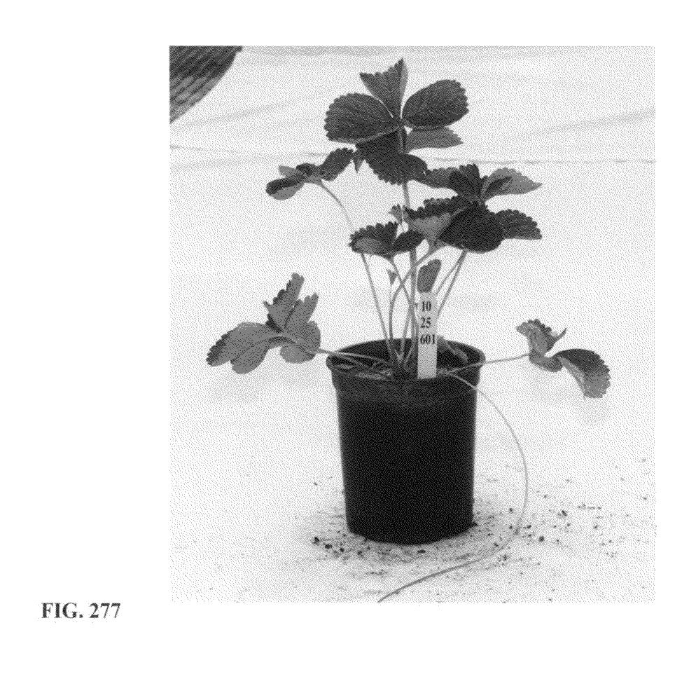

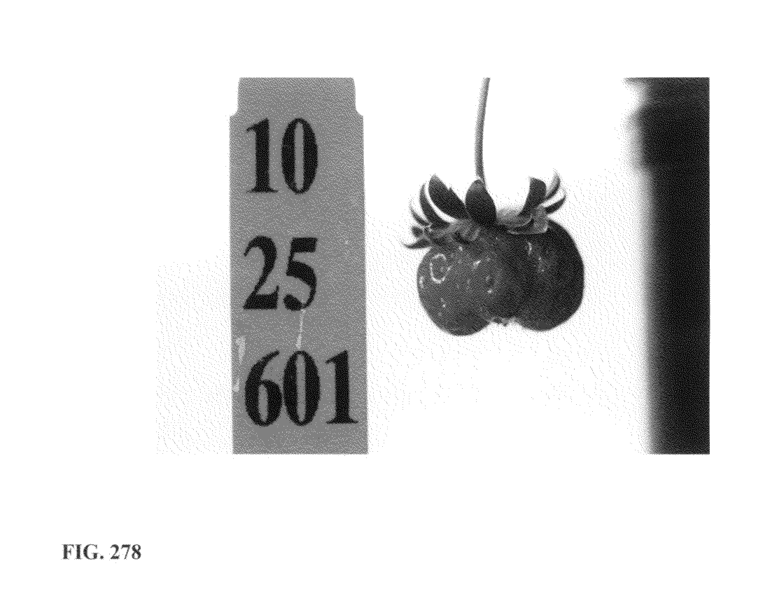

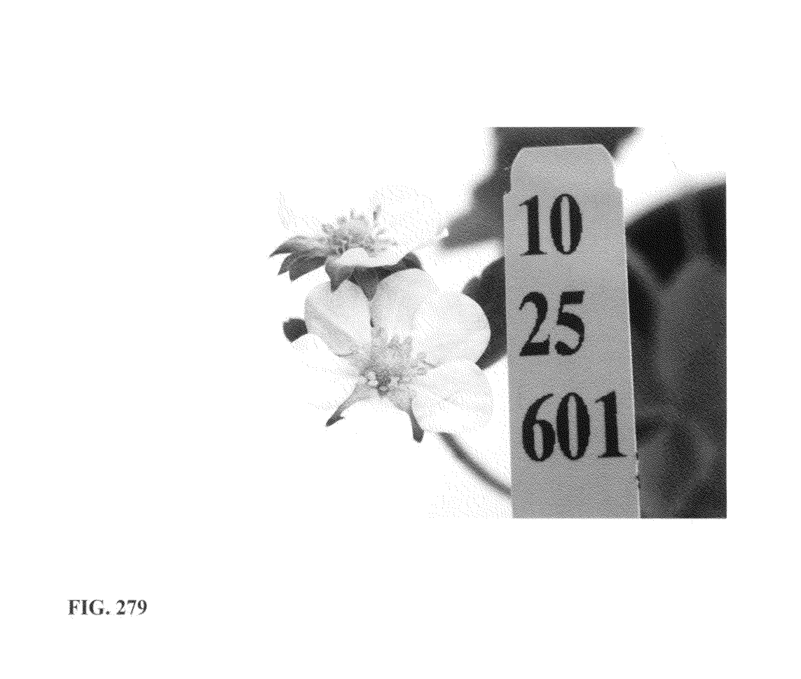

Cultivar ‘10.25-601’ has distinct disease/pathogen resistance, yield, appearance, size, and firmness. The plant of cultivar ‘10.25-601’ is characterized and distinguished in FIG. 277, the fruit of cultivar ‘10.25-601’ is characterized and distinguished in FIG. 278, and the flower of cultivar ‘10.25-601’ is characterized and distinguished in FIG. 279.

The following detailed description of cultivar ‘10.25-601’ is based on observations taken of plants and fruits (see FIGS. 277 and 278).

Certain characteristics of cultivar ‘10.25-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 93 provides data that characterizes the yield, appearance, size, and firmness of cultivar ‘10.25-601’.

TABLE 93

| Cultivar | Yield | App | Size | Firm |
|---|---|---|---|---|
| 10.25-601 | 2144 | 3.4 | 31.3 | 10.0 |

### Cultivar ‘10.30-1’

In certain aspects, the present invention relates to a new and distinctive day-neutral type strawberry cultivar designated as ‘10.30-1’. Cultivar ‘10.30-1’ is typical of day-neutral strawberry cultivars and produces fruit regardless of day length when treated appropriately in arid, subtropical climates.

The strawberry cultivar ‘10.30-1’ originated from a cross between two unreleased germplasm accessions ‘05.108-7’ and ‘05.65-608’. The cultivar has been asexually reproduced by runners. The properties of this cultivar were found to be transmissible by such asexual reproduction. The cultivar is stable and reproduces true to type in successive generations of asexual reproduction.

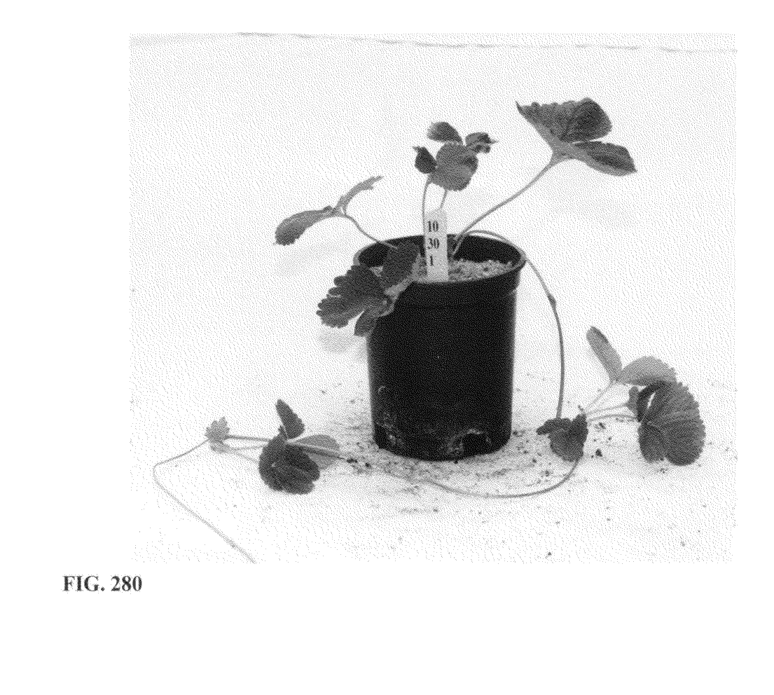

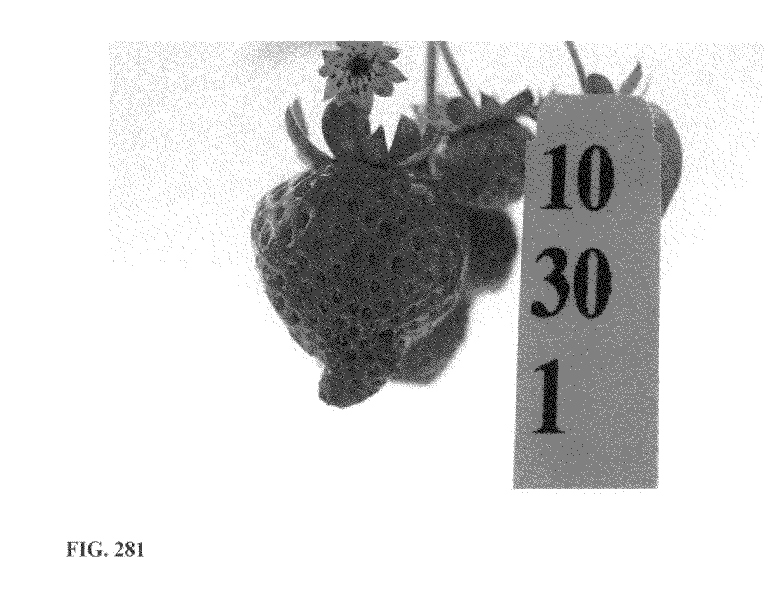

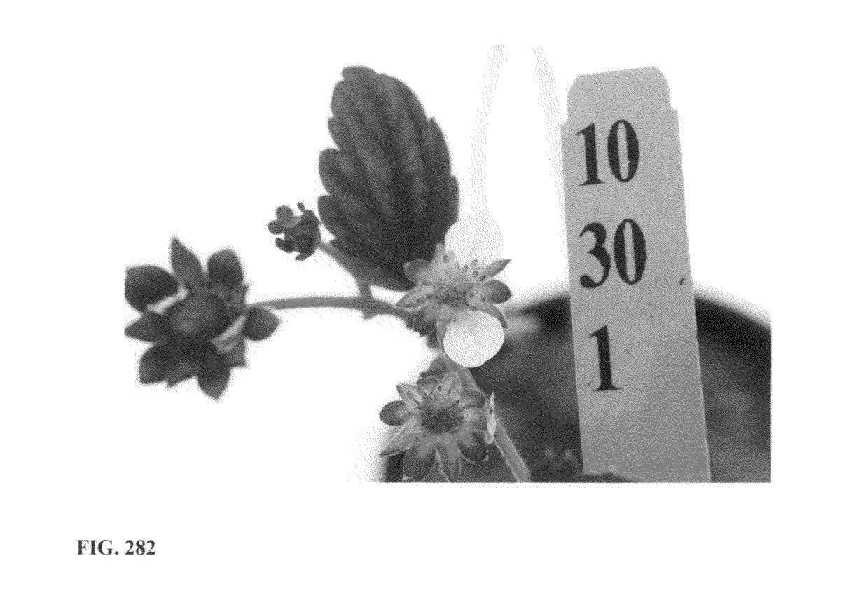

Cultivar ‘10.30-1’ has distinct disease/pathogen resistance, yield, appearance, size, and firmness. The plant of cultivar ‘10.30-1’ is characterized and distinguished in FIG. 280, the fruit of cultivar ‘10.30-1’ is characterized and distinguished in FIG. 281, and the flower of cultivar ‘10.30-1’ is characterized and distinguished in FIG. 282.

The following detailed description of cultivar ‘10.30-1’ is based on observations taken of plants and fruits (see FIGS. 280 and 281).

Certain characteristics of cultivar ‘10.30-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 94 provides data that characterizes the yield, appearance, size, and firmness of cultivar ‘10.30-1’.

TABLE 94

| Cultivar | Yield | App | Size | Firm |
|---|---|---|---|---|
| 10.30-1 | 1599 | 3.4 | 29.6 | 12.8 |

### Cultivar ‘10.31-602’

In certain aspects, the present invention relates to a new and distinctive short-day type strawberry cultivar designated as ‘10.31-602’. Cultivar ‘10.31-602’ is typical of short-day strawberry cultivars and produces fruit over an extended period when treated appropriately in arid, subtropical climates.

The strawberry cultivar ‘10.31-602’ originated from a cross between two unreleased germplasm accessions ‘06.16-602’ and ‘05.65-608’. The cultivar has been asexually reproduced by runners. The properties of this cultivar were found to be transmissible by such asexual reproduction. The cultivar is stable and reproduces true to type in successive generations of asexual reproduction.

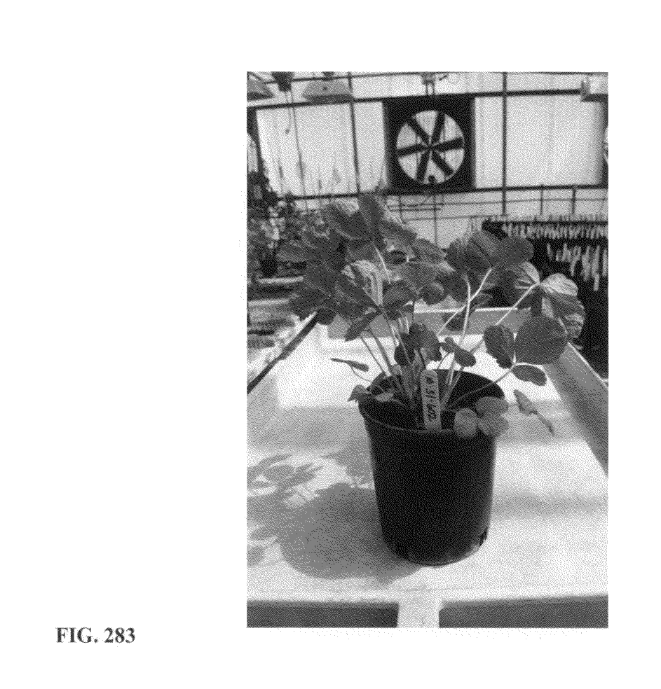

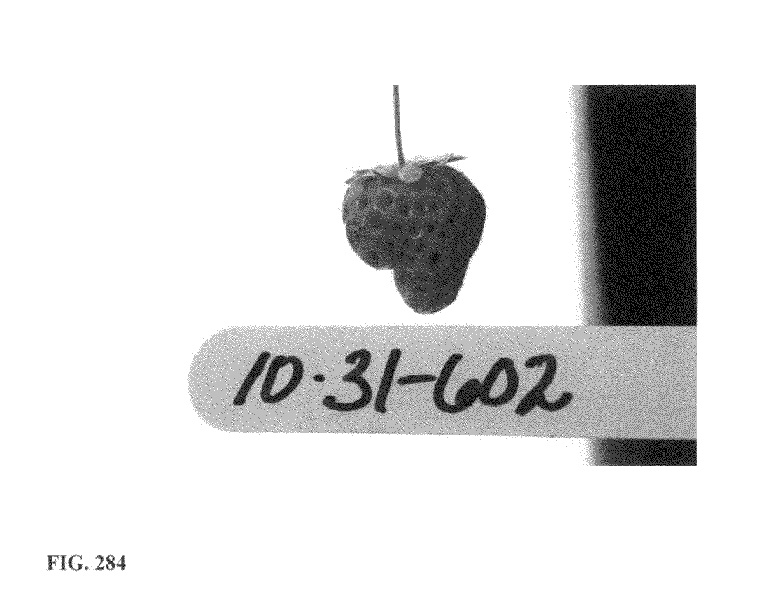

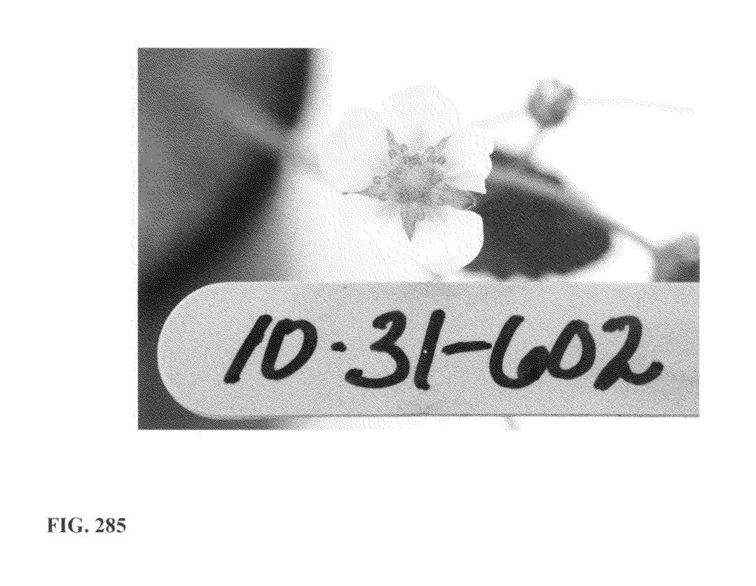

Cultivar ‘10.31-602’ has distinct disease/pathogen resistance, yield, appearance, size, and firmness. The plant of cultivar ‘10.31-602’ is characterized and distinguished in FIG. 283, the fruit of cultivar ‘10.31-602’ is characterized and distinguished in FIG. 284, and the flower of cultivar ‘10.31-602’ is characterized and distinguished in FIG. 285.

The following detailed description of cultivar ‘10.31-602’ is based on observations taken of plants and fruits (see FIGS. 283 and 284).

Certain characteristics of cultivar ‘10.31-602’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 95 provides data that characterizes the yield, appearance, size, and firmness of cultivar ‘10.31-602’.

TABLE 95

| Cultivar | Yield | App | Size | Firm |
|---|---|---|---|---|
| 10.31-602 | 1482 | 3.6 | 35.8 | 9.7 |

### Cultivar ‘10.37-604’

In certain aspects, the present invention relates to a new and distinctive short-day type strawberry cultivar designated as ‘10.37-604’. Cultivar ‘10.37-604’ is typical of short-day strawberry cultivars and produces fruit over an extended period when treated appropriately in arid, subtropical climates.

The strawberry cultivar ‘10.37-604’ originated from a cross between two unreleased germplasm accessions ‘06.16-602’ and ‘06.50-611’. The cultivar has been asexually reproduced by runners. The properties of this cultivar were found to be transmissible by such asexual reproduction. The cultivar is stable and reproduces true to type in successive generations of asexual reproduction.

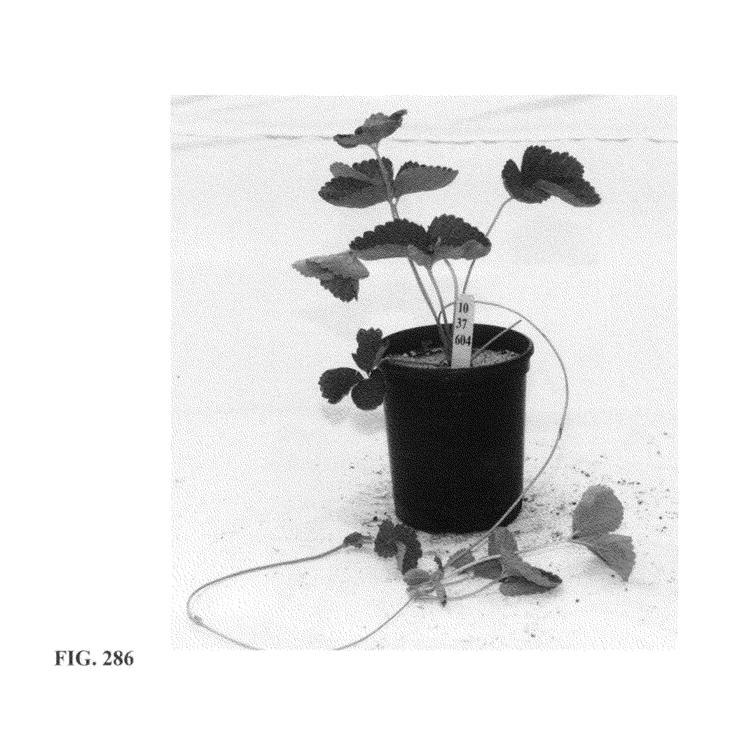

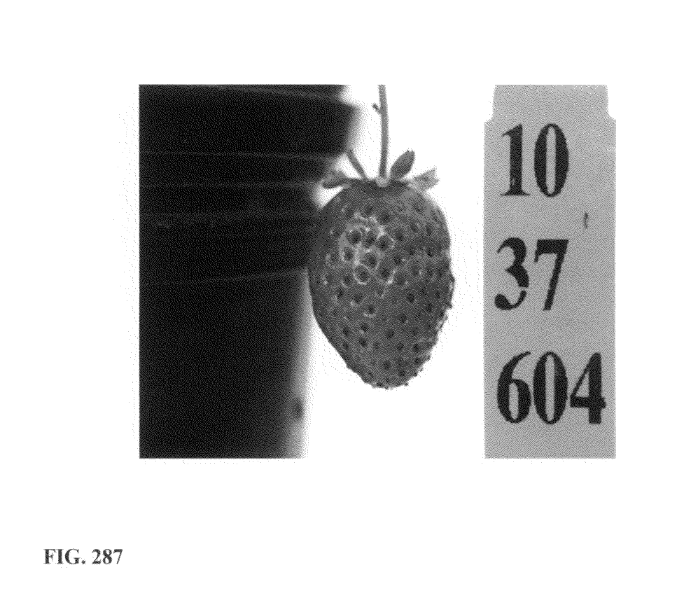

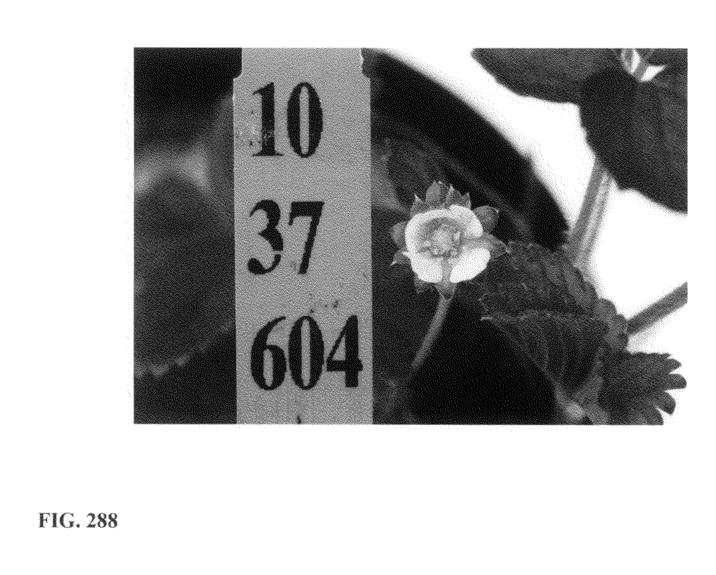

Cultivar ‘10.37-604’ has distinct disease/pathogen resistance, yield, appearance, size, and firmness. The plant of cultivar ‘10.37-604’ is characterized and distinguished in FIG. 286, the fruit of cultivar ‘10.37-604’ is characterized and distinguished in FIG. 287, and the flower of cultivar ‘10.37-604’ is characterized and distinguished in FIG. 288.

The following detailed description of cultivar ‘10.37-604’ is based on observations taken of plants and fruits (see FIGS. 286 and 287).

Certain characteristics of cultivar ‘10.37-604’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 96 provides data that characterizes the yield, appearance, size, and firmness of cultivar ‘10.37-604’.

TABLE 96

| Cultivar | Yield | App | Size | Firm |
|---|---|---|---|---|
| 10.37-604 | 2463 | 3.9 | 30.3 | 13.5 |

### Cultivar ‘10.38-601’

In certain aspects, the present invention relates to a new and distinctive short-day type strawberry cultivar designated as ‘10.38-601’. Cultivar ‘10.38-601’ is typical of short-day strawberry cultivars and produces fruit over an extended period when treated appropriately in arid, subtropical climates.

The strawberry cultivar ‘10.38-601’ originated from a cross between two unreleased germplasm accessions ‘07.64-3’ and ‘06.50-611’. The cultivar has been asexually reproduced by runners. The properties of this cultivar were found to be transmissible by such asexual reproduction. The cultivar is stable and reproduces true to type in successive generations of asexual reproduction.

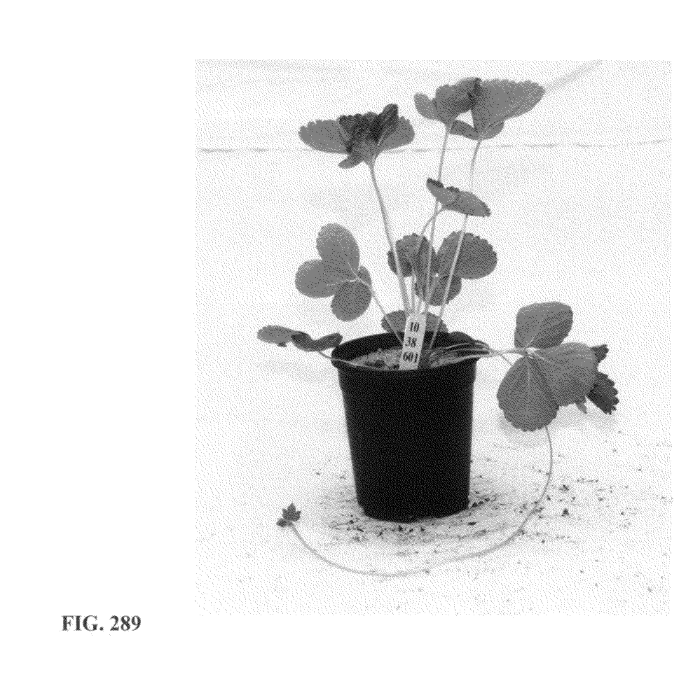

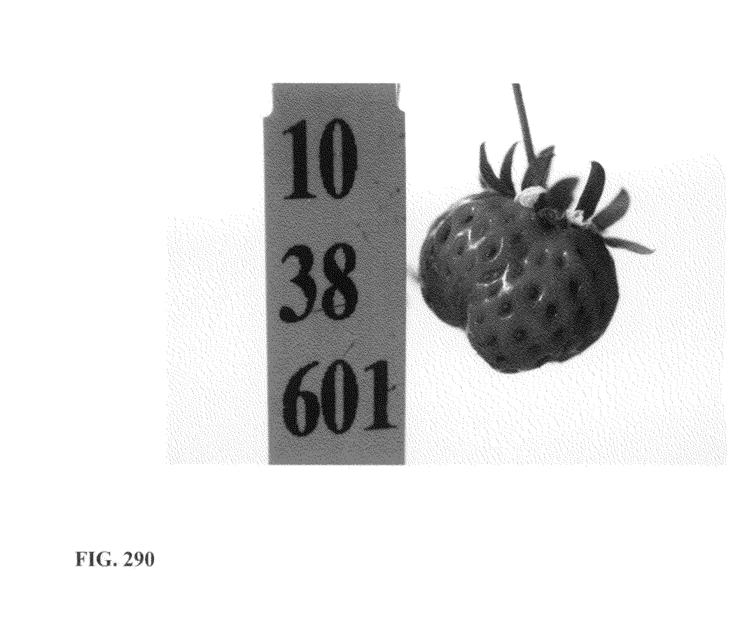

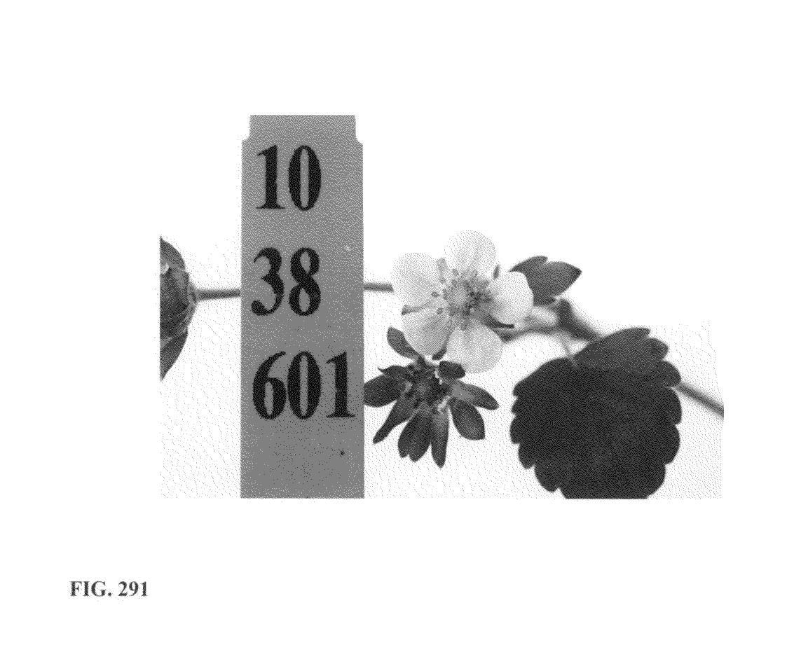

Cultivar ‘10.38-601’ has distinct disease/pathogen resistance, yield, appearance, size, and firmness. The plant of cultivar ‘10.38-601’ is characterized and distinguished in FIG. 289, the fruit of cultivar ‘10.38-601’ is characterized and distinguished in FIG. 290, and the flower of cultivar ‘10.38-601’ is characterized and distinguished in FIG. 291.

The following detailed description of cultivar ‘10.38-601’ is based on observations taken of plants and fruits (see FIGS. 289 and 290).

Certain characteristics of cultivar ‘10.38-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 97 provides data that characterizes the yield, appearance, size, and firmness of cultivar ‘10.38-601’.

TABLE 97

| Cultivar | Yield | App | Size | Firm |
|---|---|---|---|---|
| 10.38-601 | 2843 | 3.6 | 33.1 | 10.7 |

### Cultivar ‘10.70-611’

In certain aspects, the present invention relates to a new and distinctive short-day type strawberry cultivar designated as ‘10.70-611’. Cultivar ‘10.70-611’ is typical of short-day strawberry cultivars and produces fruit over an extended period when treated appropriately in arid, subtropical climates.

The strawberry cultivar ‘10.70-611’ originated from a cross between two unreleased germplasm accessions ‘06.20-605’ and ‘06.43-603’. The cultivar has been asexually reproduced by runners. The properties of this cultivar were found to be transmissible by such asexual reproduction. The cultivar is stable and reproduces true to type in successive generations of asexual reproduction.

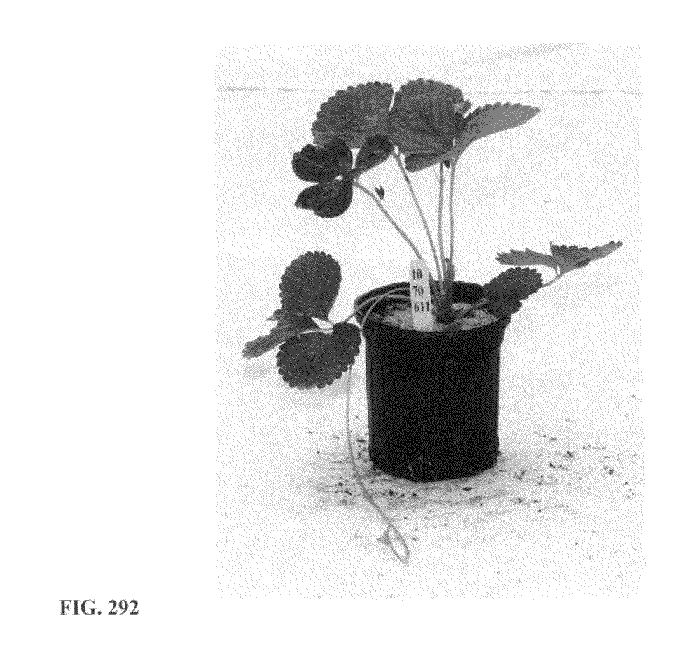

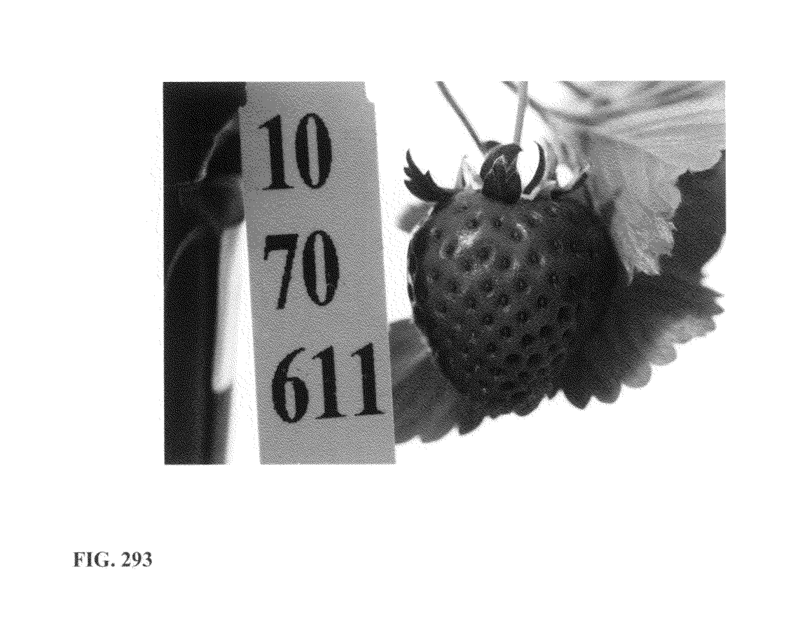

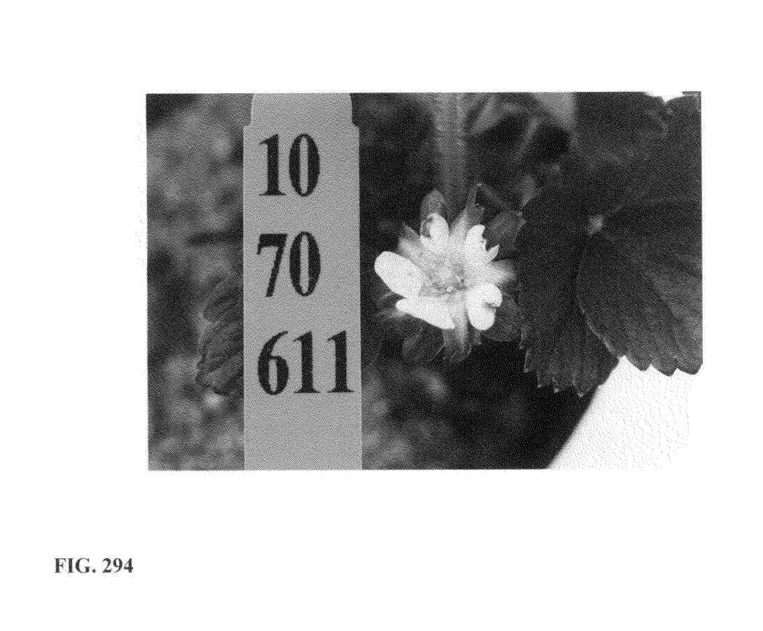

Cultivar ‘10.70-611’ has distinct disease/pathogen resistance, yield, appearance, size, and firmness. The plant of cultivar ‘10.70-611’ is characterized and distinguished in FIG. 292, the fruit of cultivar ‘10.70-611’ is characterized and distinguished in FIG. 293, and the flower of cultivar ‘10.70-611’ is characterized and distinguished in FIG. 294.

The following detailed description of cultivar ‘10.70-611’ is based on observations taken of plants and fruits (see FIGS. 292 and 293).

Certain characteristics of cultivar ‘10.70-611’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 98 provides data that characterizes the yield, appearance, size, and firmness of cultivar ‘10.70-611’.

TABLE 98

| Cultivar | Yield | App | Size | Firm |
|---|---|---|---|---|
| 10.70-611 | 2319 | 3.2 | 29.9 | 11.5 |

### Cultivar ‘10.77-601’

In certain aspects, the present invention relates to a new and distinctive short-day type strawberry cultivar designated as ‘10.77-601’. Cultivar ‘10.77-601’ is typical of short-day strawberry cultivars and produces fruit over an extended period when treated appropriately in arid, subtropical climates.

The strawberry cultivar ‘10.77-601’ originated from a cross between two unreleased germplasm accessions ‘06.66-603’ and ‘06.51-609’. The cultivar has been asexually reproduced by runners. The properties of this cultivar were found to be transmissible by such asexual reproduction. The cultivar is stable and reproduces true to type in successive generations of asexual reproduction.

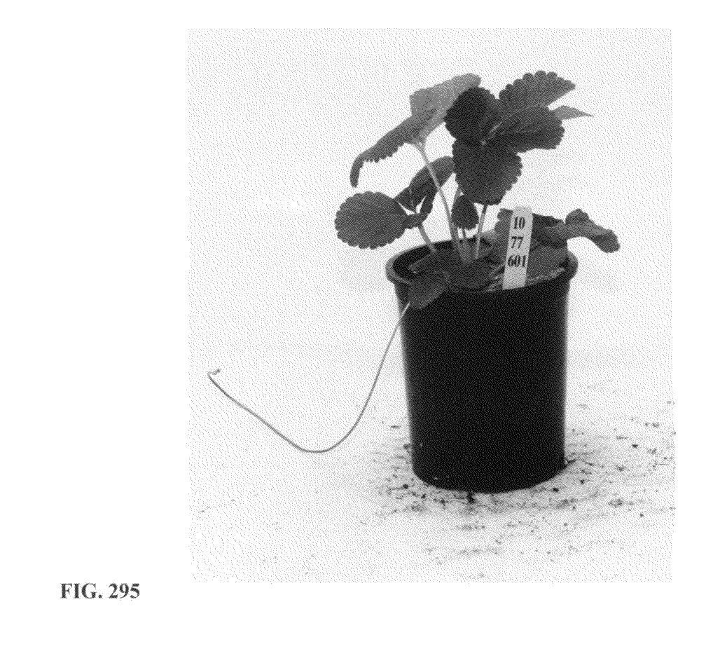

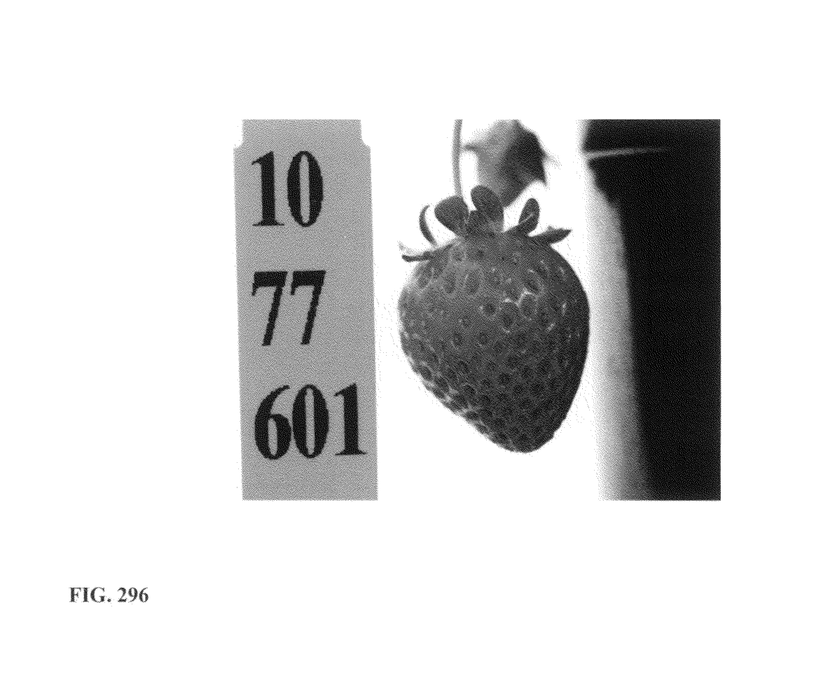

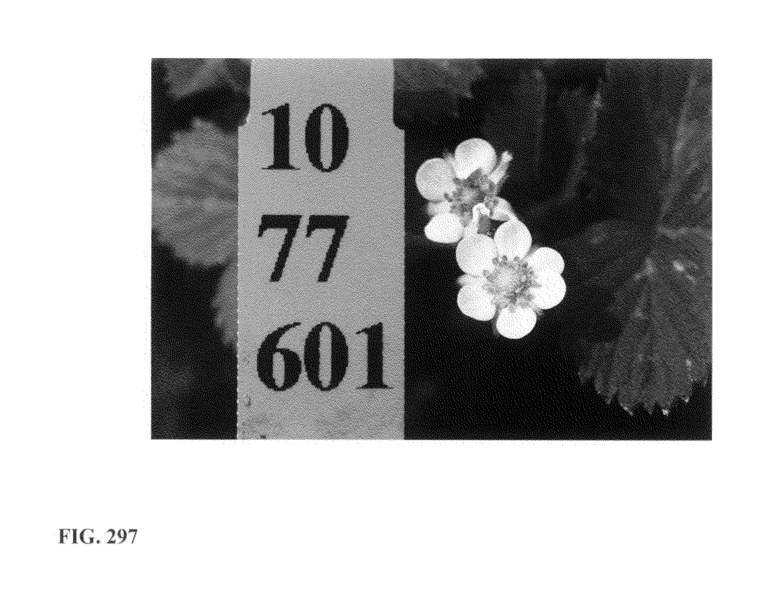

Cultivar ‘10.77-601’ has distinct disease/pathogen resistance, yield, appearance, size, and firmness. The plant of cultivar ‘10.77-601’ is characterized and distinguished in FIG. 295, the fruit of cultivar ‘10.77-601’ is characterized and distinguished in FIG. 296, and the flower of cultivar ‘10.77-601’ is characterized and distinguished in FIG. 297.

The following detailed description of cultivar ‘10.77-601’ is based on observations taken of plants and fruits (see FIGS. 295 and 296).

Certain characteristics of cultivar ‘10.77-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 99 provides data that characterizes the yield, appearance, size, and firmness of cultivar ‘10.77-601’.

TABLE 99

| Cultivar | Yield | App | Size | Firm |
|---|---|---|---|---|
| 10.77-601 | 1159 | 3.6 | 36.4 | 9.7 |

### Cultivar ‘10.78-603’

In certain aspects, the present invention relates to a new and distinctive short-day type strawberry cultivar designated as ‘10.78-603’. Cultivar ‘10.78-603’ is typical of short-day strawberry cultivars and produces fruit over an extended period when treated appropriately in arid, subtropical climates.

The strawberry cultivar ‘10.78-603’ originated from a cross between two unreleased germplasm accessions ‘06.137-2’ and ‘06.51-609’. The cultivar has been asexually reproduced by runners. The properties of this cultivar were found to be transmissible by such asexual reproduction. The cultivar is stable and reproduces true to type in successive generations of asexual reproduction.

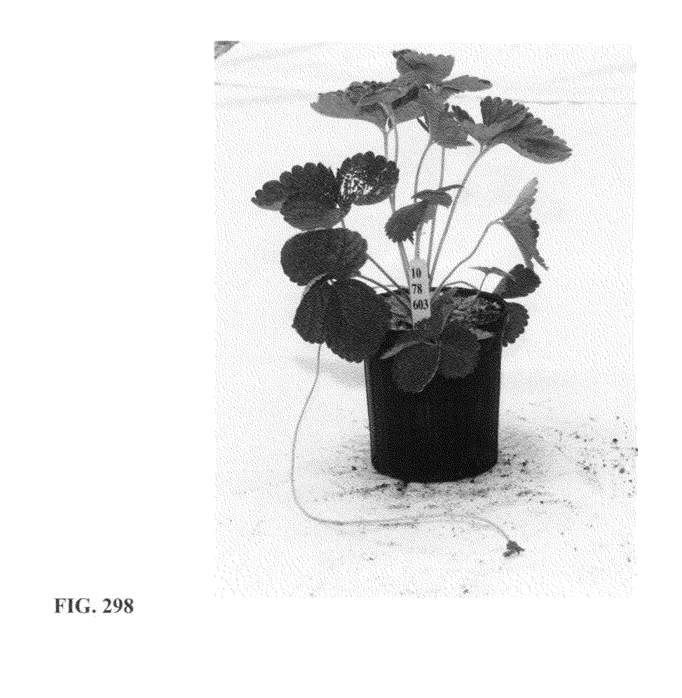

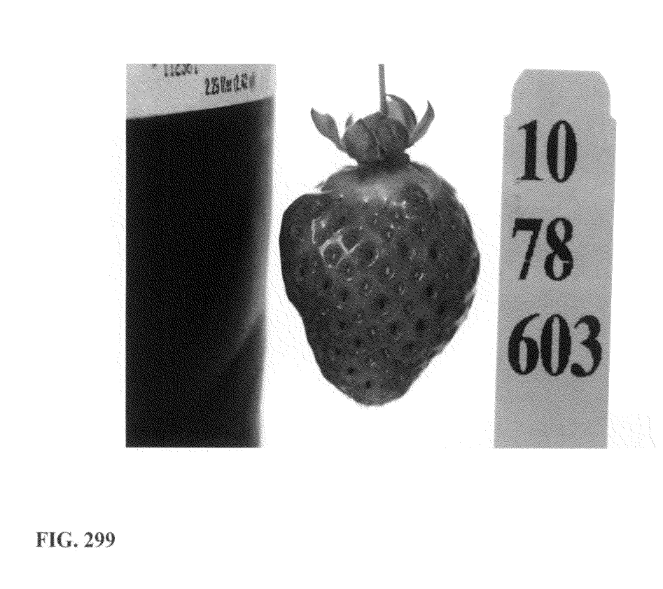

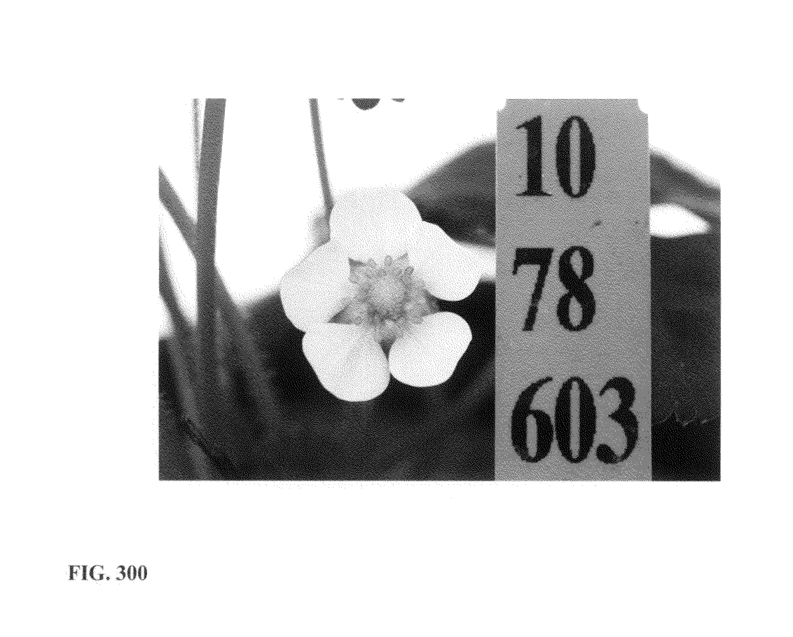

Cultivar ‘10.78-603’ has distinct disease/pathogen resistance, yield, appearance, size, and firmness. The plant of cultivar ‘10.78-603’ is characterized and distinguished in FIG. 298, the fruit of cultivar ‘10.78-603’ is characterized and distinguished in FIG. 299, and the flower of cultivar ‘10.78-603’ is characterized and distinguished in FIG. 300.

The following detailed description of cultivar ‘10.78-603’ is based on observations taken of plants and fruits (see FIGS. 298 and 299).

Certain characteristics of cultivar ‘10.78-603’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 100 provides data that characterizes the yield, appearance, size, and firmness of cultivar ‘10.78-603’.

TABLE 100

| Cultivar | Yield | App | Size | Firm |
|---|---|---|---|---|
| 10.78-603 | 1435 | 3.5 | 47.9 | 10.9 |

### Cultivar ‘10.80-605’

In certain aspects, the present invention relates to a new and distinctive short-day type strawberry cultivar designated as ‘10.80-605’. Cultivar ‘10.80-605’ is typical of short-day strawberry cultivars and produces fruit over an extended period when treated appropriately in arid, subtropical climates.

The strawberry cultivar ‘10.80-605’ originated from a cross between cultivar ‘Merced’ and unreleased germplasm accession ‘06.51-609’. The cultivar has been asexually reproduced by runners. The properties of this cultivar were found to be transmissible by such asexual reproduction. The cultivar is stable and reproduces true to type in successive generations of asexual reproduction.

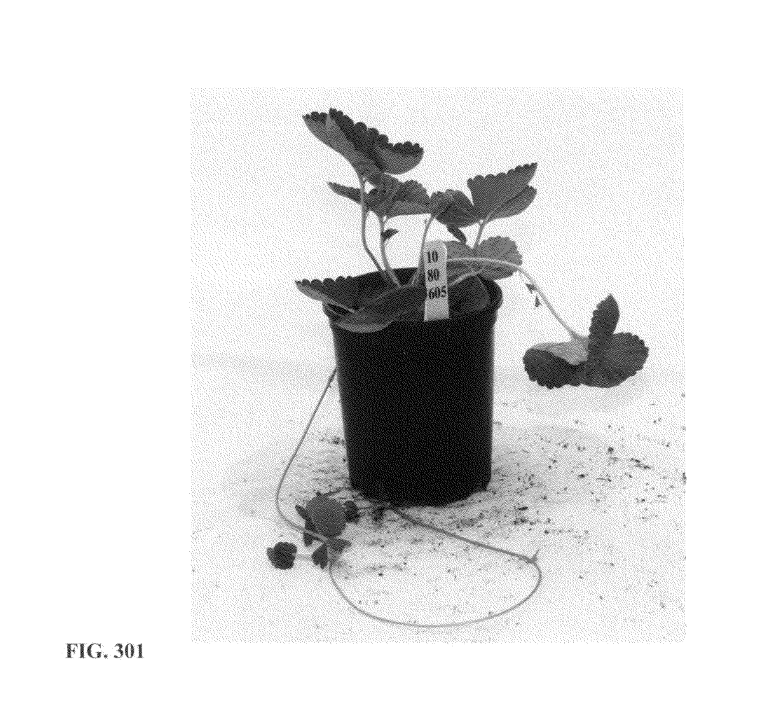

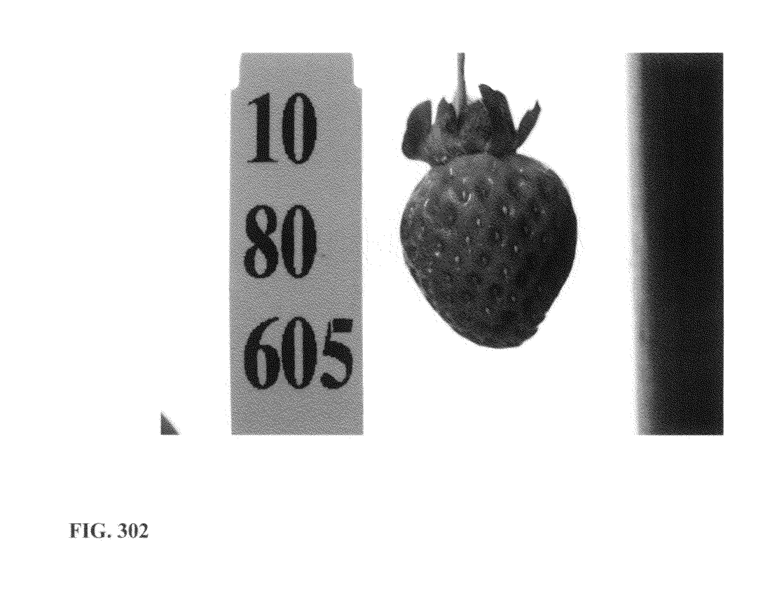

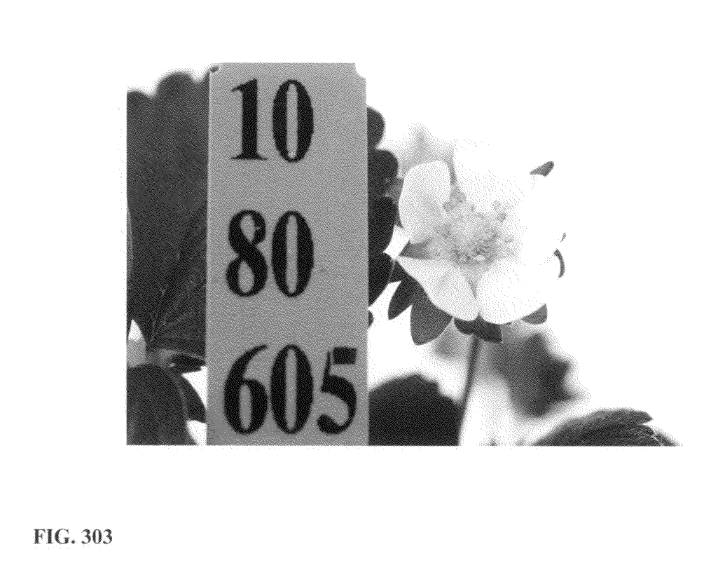

Cultivar ‘10.80-605’ has distinct disease/pathogen resistance, yield, appearance, size, and firmness. The plant of cultivar ‘10.80-605’ is characterized and distinguished in FIG. 301, the fruit of cultivar ‘10.80-605’ is characterized and distinguished in FIG. 302, and the flower of cultivar ‘10.80-605’ is characterized and distinguished in FIG. 303.

The following detailed description of cultivar ‘10.80-605’ is based on observations taken of plants and fruits (see FIGS. 301 and 302).

Certain characteristics of cultivar ‘10.80-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 101 provides data that characterizes the yield, appearance, size, and firmness of cultivar ‘10.80-605’.

TABLE 101

| Cultivar | Yield | App | Size | Firm |
|---|---|---|---|---|
| 10.80-605 | 1211 | 4.0 | 38.8 | 10.9 |

### Cultivar ‘10.81-1’

In certain aspects, the present invention relates to a new and distinctive short-day type strawberry cultivar designated as ‘10.81-1’. Cultivar ‘10.81-1’ is typical of short-day strawberry cultivars and produces fruit over an extended period when treated appropriately in arid, subtropical climates.

The strawberry cultivar ‘10.81-1’ originated from a cross between two unreleased germplasm accessions ‘07.141-1’ and ‘06.51-609’. The cultivar has been asexually reproduced by runners. The properties of this cultivar were found to be transmissible by such asexual reproduction. The cultivar is stable and reproduces true to type in successive generations of asexual reproduction.

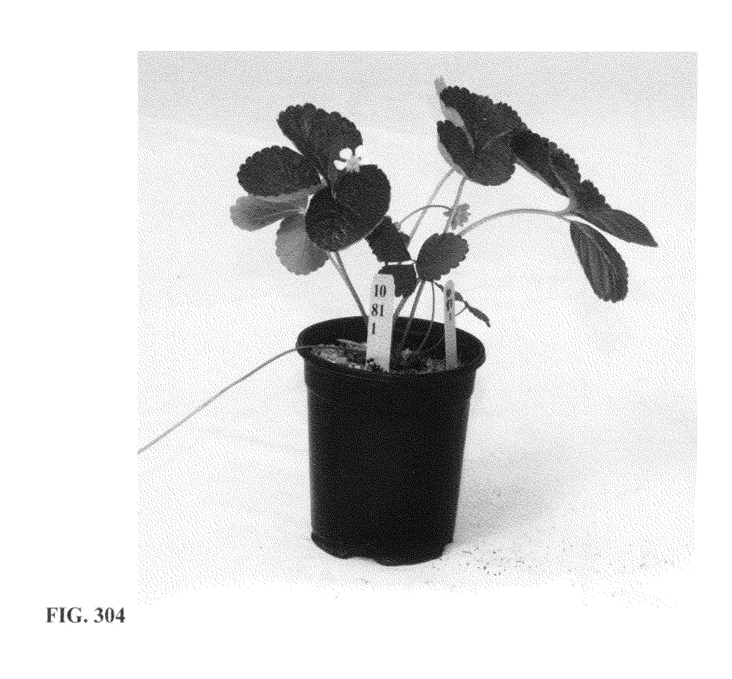

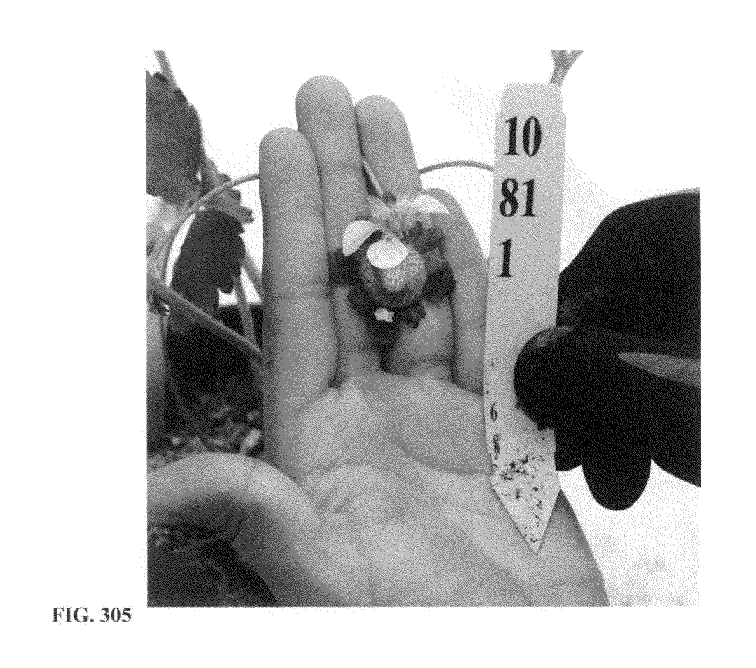

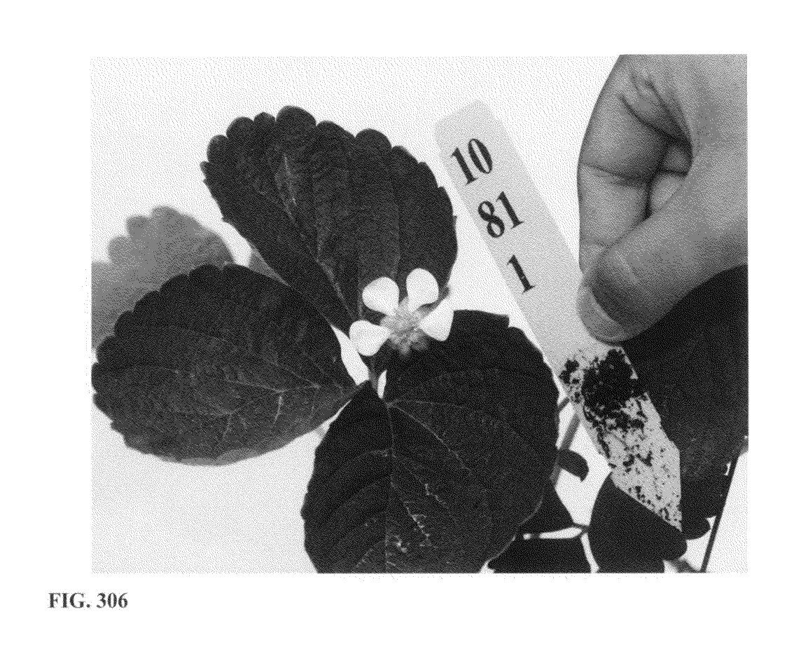

Cultivar ‘10.81-1’ has distinct disease/pathogen resistance, yield, appearance, size, and firmness. The plant of cultivar ‘10.81-1’ is characterized and distinguished in FIG. 304, the fruit of cultivar ‘10.81-1’ is characterized and distinguished in FIG. 305, and the flower of cultivar ‘10.81-1’ is characterized and distinguished in FIG. 306.

The following detailed description of cultivar ‘10.81-1’ is based on observations taken of plants and fruits (see FIGS. 304 and 305).

Certain characteristics of cultivar ‘10.81-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 102 provides data that characterizes the yield, appearance, size, and firmness of cultivar ‘10.81-1’.

TABLE 102

| Cultivar | Yield | App | Size | Firm |
|---|---|---|---|---|
| 10.81-1 | 2237 | 3.6 | 33.7 | 10.0 |

### Cultivar ‘10.82-601’

In certain aspects, the present invention relates to a new and distinctive short-day type strawberry cultivar designated as ‘10.82-601’. Cultivar ‘10.82-601’ is typical of short-day strawberry cultivars and produces fruit over an extended period when treated appropriately in arid, subtropical climates.

The strawberry cultivar ‘10.82-601’ originated from a cross between two unreleased germplasm accessions ‘06.20-605’ and ‘06.111-1’. The cultivar has been asexually reproduced by runners. The properties of this cultivar were found to be transmissible by such asexual reproduction. The cultivar is stable and reproduces true to type in successive generations of asexual reproduction.

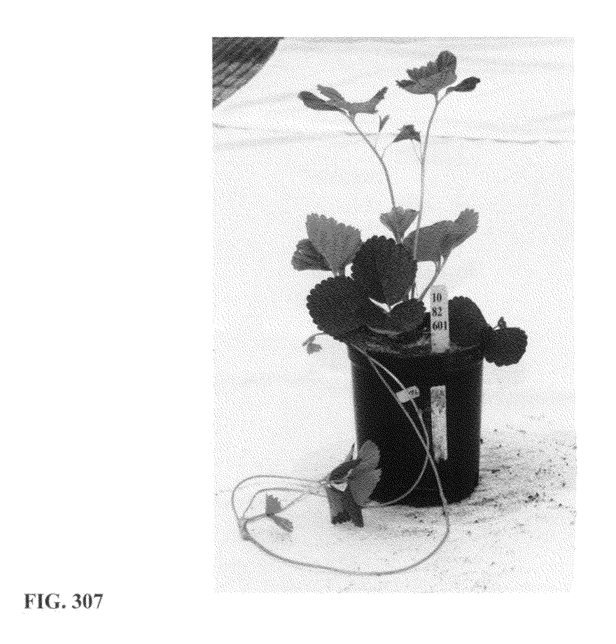

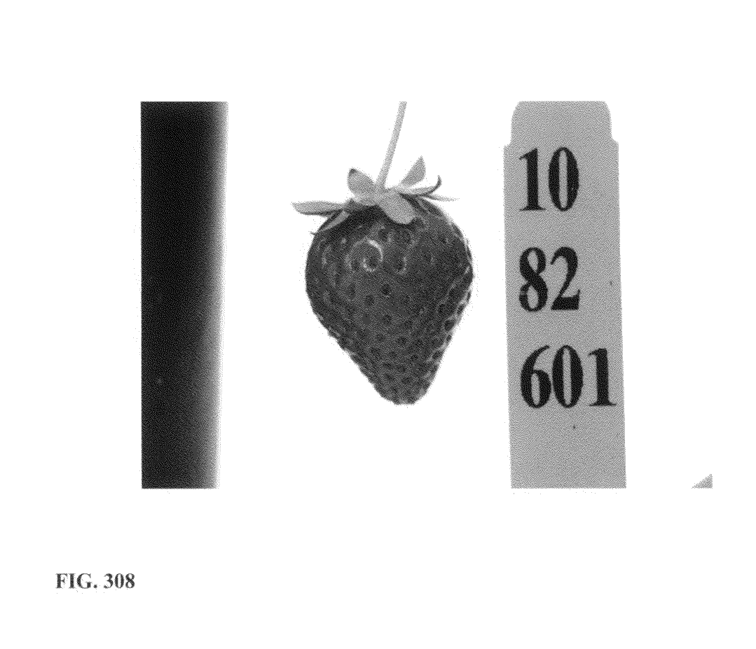

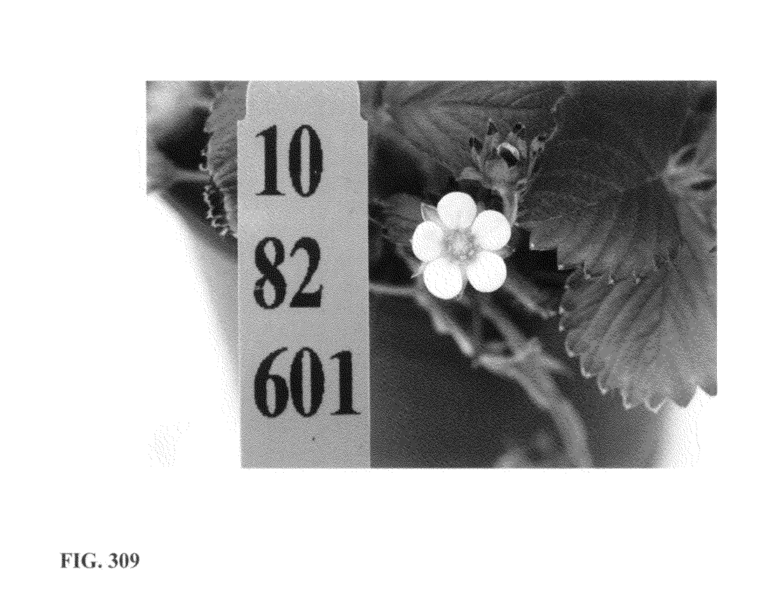

Cultivar ‘10.82-601’ has distinct disease/pathogen resistance, yield, appearance, size, and firmness. The plant of cultivar ‘10.82-601’ is characterized and distinguished in FIG. 307, the fruit of cultivar ‘10.82-601’ is characterized and distinguished in FIG. 308, and the flower of cultivar ‘10.82-601’ is characterized and distinguished in FIG. 309.

The following detailed description of cultivar ‘10.82-601’ is based on observations taken of plants and fruits (see FIGS. 307 and 308).

Certain characteristics of cultivar ‘10.82-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 103 provides data that characterizes the yield, appearance, size, and firmness of cultivar ‘10.82-601’.

TABLE 103

| Cultivar | Yield | App | Size | Firm |
|---|---|---|---|---|
| 10.82-601 | 1317 | 3.9 | 54.7 | 10.9 |

### Cultivar ‘10.87-2’

In certain aspects, the present invention relates to a new and distinctive short-day type strawberry cultivar designated as ‘10.87-2’. Cultivar ‘10.87-2’ is typical of short-day strawberry cultivars and produces fruit over an extended period when treated appropriately in arid, subtropical climates.

The strawberry cultivar ‘10.87-2’ originated from a cross between two unreleased germplasm accessions ‘07.141-1’ and ‘06.111-1’. The cultivar has been asexually reproduced by runners. The properties of this cultivar were found to be transmissible by such asexual reproduction. The cultivar is stable and reproduces true to type in successive generations of asexual reproduction.

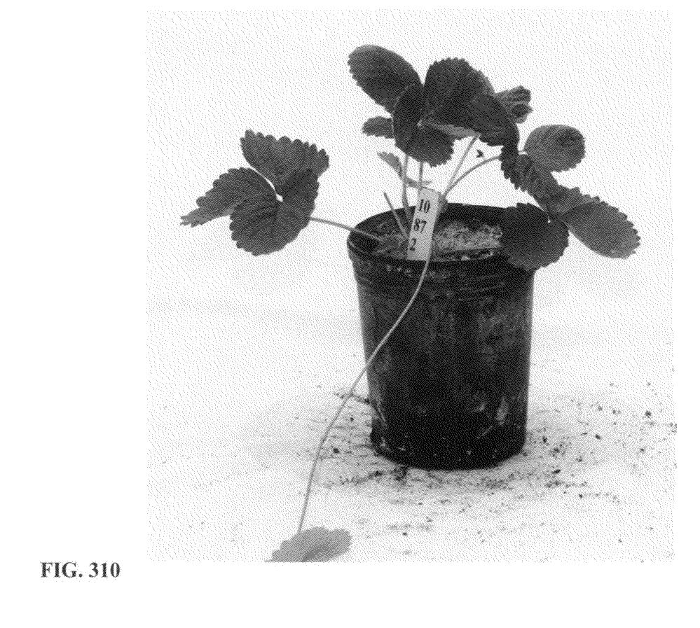

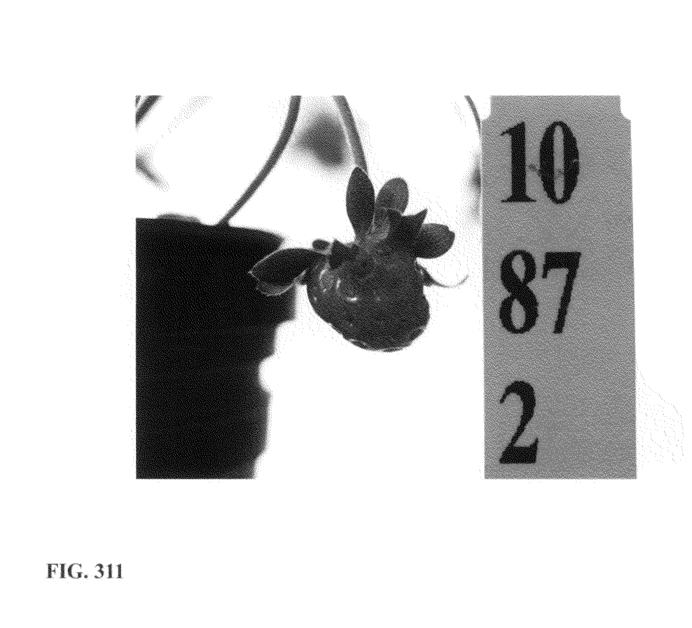

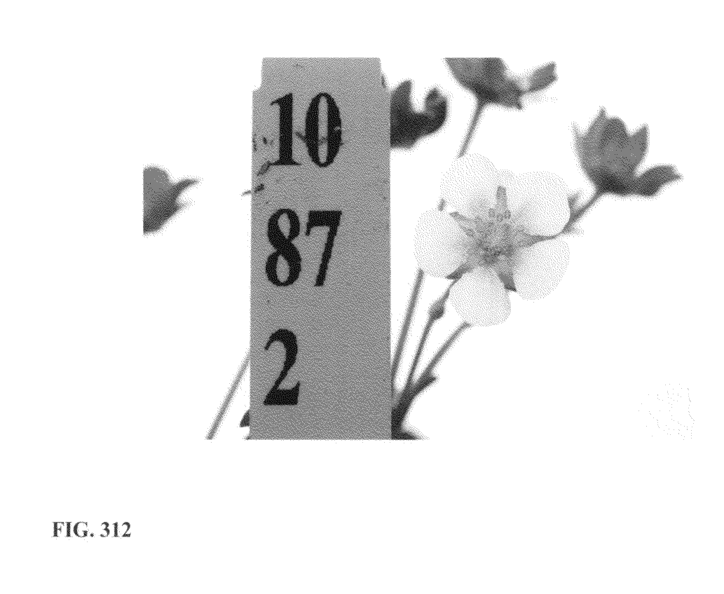

Cultivar ‘10.87-2’ has distinct disease/pathogen resistance, yield, appearance, size, and firmness. The plant of cultivar ‘10.87-2’ is characterized and distinguished in FIG. 310, the fruit of cultivar ‘10.87-2’ is characterized and distinguished in FIG. 311, and the flower of cultivar ‘10.87-2’ is characterized and distinguished in FIG. 312.

The following detailed description of cultivar ‘10.87-2’ is based on observations taken of plants and fruits (see FIGS. 310 and 311).

Certain characteristics of cultivar ‘10.87-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 104 provides data that characterizes the yield, appearance, size, and firmness of cultivar ‘10.87-2’.

TABLE 104

| Cultivar | Yield | App | Size | Firm |
|---|---|---|---|---|
| 10.87-2 | 1694 | 3.3 | 34.3 | 10.3 |

### Cultivar ‘10.99-1’

In certain aspects, the present invention relates to a new and distinctive day-neutral type strawberry cultivar designated as ‘10.99-1’. Cultivar ‘10.99-1’ is typical of day-neutral strawberry cultivars and produces fruit regardless of day length when treated appropriately in arid, subtropical climates.

The strawberry cultivar ‘10.99-1’ originated from a cross between two unreleased germplasm accessions ‘05.206-5’ and ‘06.51-609’. The cultivar has been asexually reproduced by runners. The properties of this cultivar were found to be transmissible by such asexual reproduction. The cultivar is stable and reproduces true to type in successive generations of asexual reproduction.

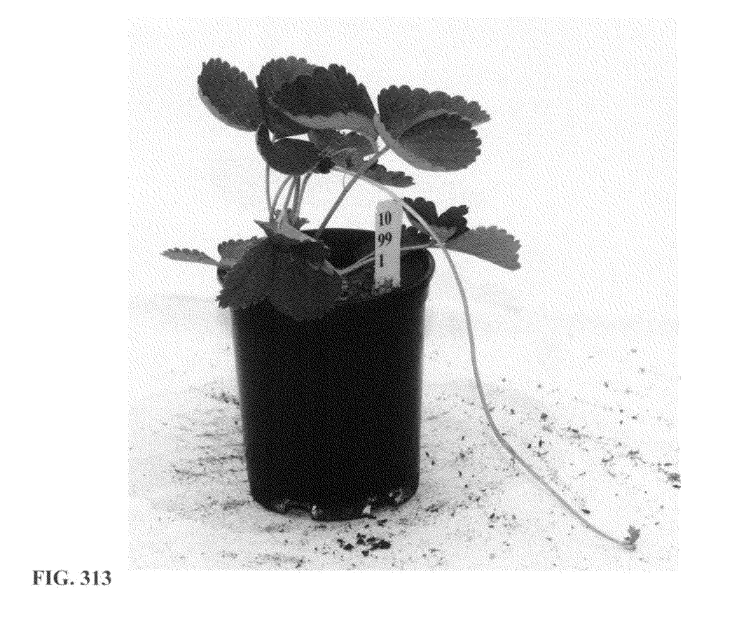

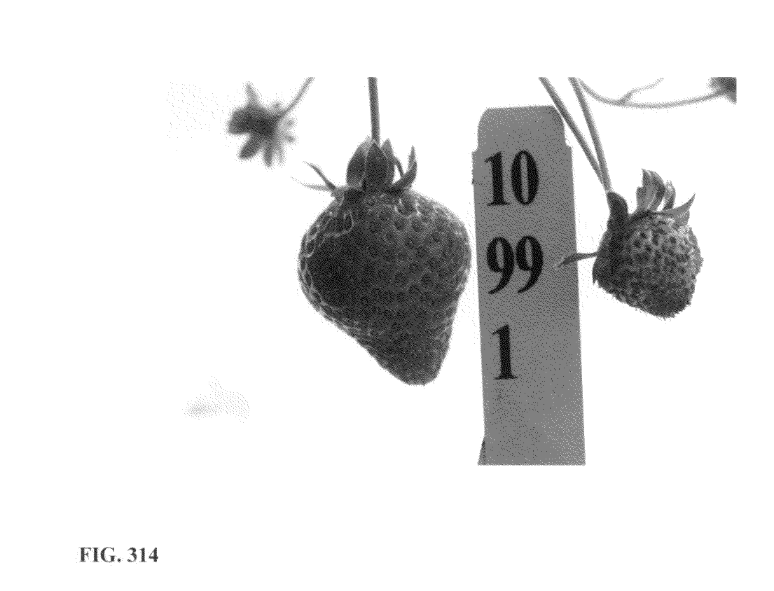

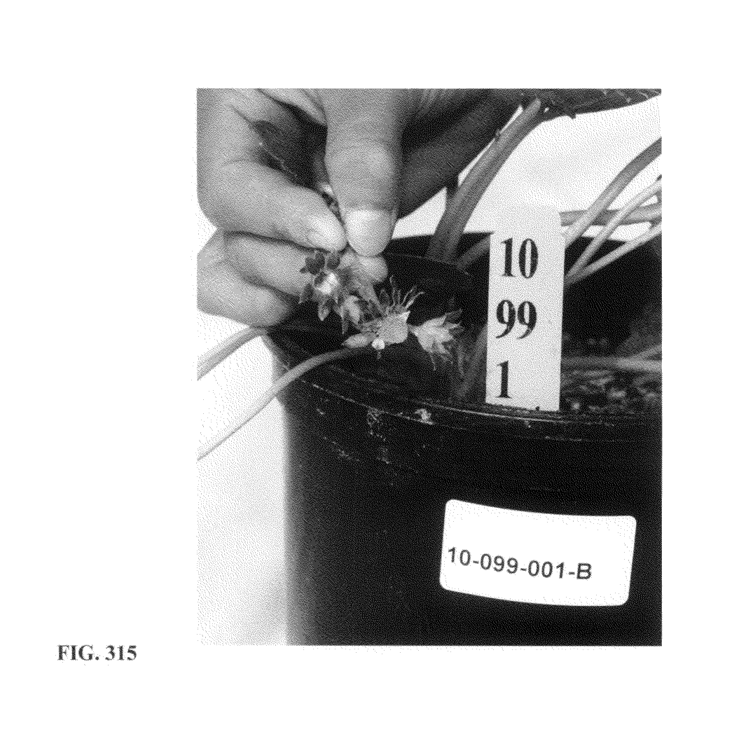

Cultivar ‘10.99-1’ has distinct disease/pathogen resistance, yield, appearance, size, and firmness. The plant of cultivar ‘10.99-1’ is characterized and distinguished in FIG. 313, the fruit of cultivar ‘10.99-1’ is characterized and distinguished in FIG. 314, and the flower of cultivar ‘10.99-1’ is characterized and distinguished in FIG. 315.

The following detailed description of cultivar ‘10.99-1’ is based on observations taken of plants and fruits (see FIGS. 313 and 314).

Certain characteristics of cultivar ‘10.99-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 105 provides data that characterizes the yield, appearance, size, and firmness of cultivar ‘10.99-1’.

TABLE 105

| Cultivar | Yield | App | Size | Firm |
|---|---|---|---|---|
| 10.99-1 | 2326 | 4.2 | 32.5 | 14.0 |

### Cultivar ‘10.105-1’

In certain aspects, the present invention relates to a new and distinctive day-neutral type strawberry cultivar designated as ‘10.105-1’. Cultivar ‘10.105-1’ is typical of day-neutral strawberry cultivars and produces fruit regardless of day length when treated appropriately in arid, subtropical climates.

The strawberry cultivar ‘10.105-1’ originated from a cross between two unreleased germplasm accessions ‘05.132-1’ and ‘07.92-3’. The cultivar has been asexually reproduced by runners. The properties of this cultivar were found to be transmissible by such asexual reproduction. The cultivar is stable and reproduces true to type in successive generations of asexual reproduction.

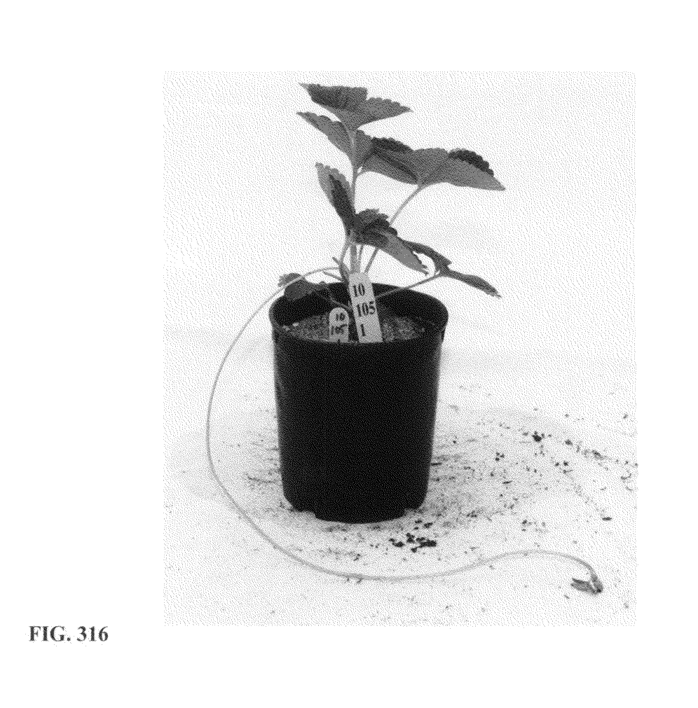

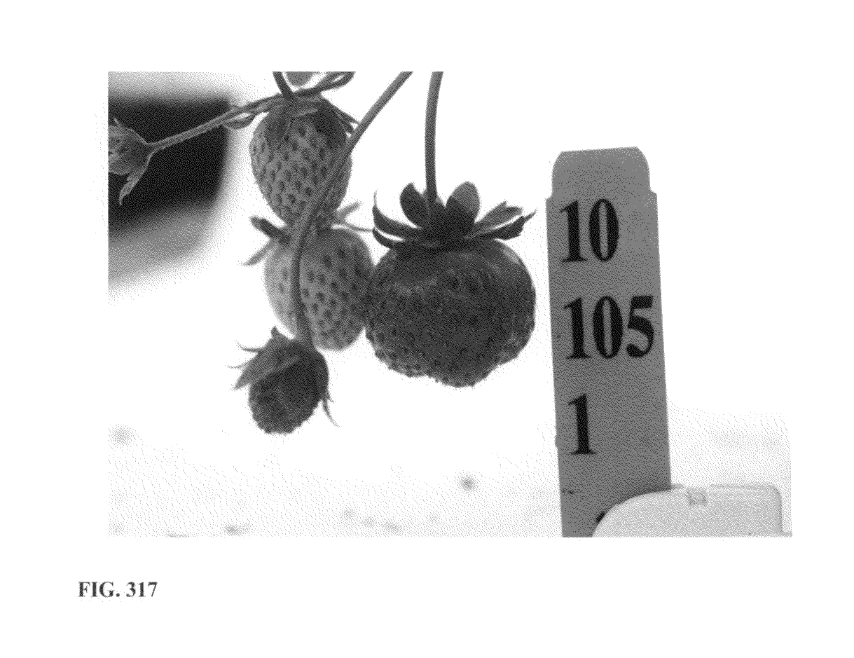

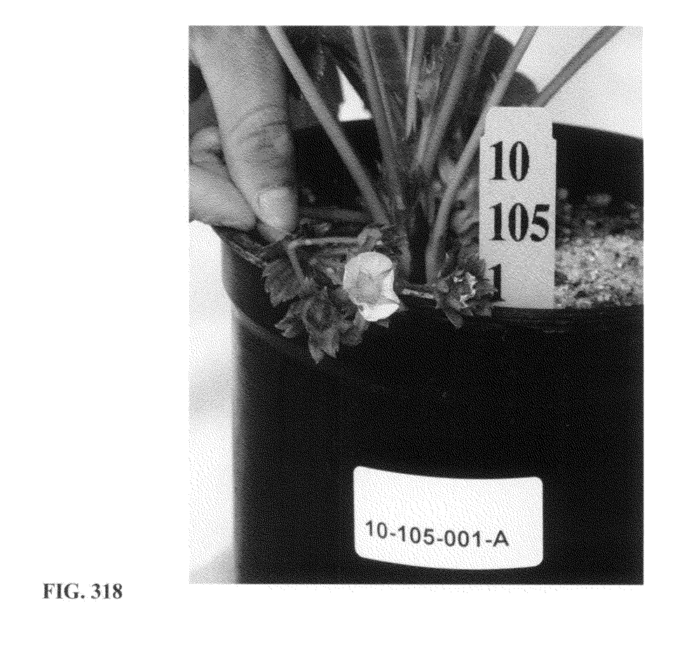

Cultivar ‘10.105-1’ has distinct disease/pathogen resistance, yield, appearance, size, and firmness. The plant of cultivar ‘10.105-1’ is characterized and distinguished in FIG. 316, the fruit of cultivar ‘10.105-1’ is characterized and distinguished in FIG. 317, and the flower of cultivar ‘10.105-1’ is characterized and distinguished in FIG. 318.

The following detailed description of cultivar ‘10.105-1’ is based on observations taken of plants and fruits (see FIGS. 316 and 317).

Certain characteristics of cultivar ‘10.105-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 106 provides data that characterizes the yield, appearance, size, and firmness of cultivar ‘10.105-1’.

TABLE 106

| Cultivar | Yield | App | Size | Firm |
|---|---|---|---|---|
| 10.105-1 | 2335 | 3.8 | 33.0 | 12.9 |

### Cultivar ‘10.106-1’

In certain aspects, the present invention relates to a new and distinctive day-neutral type strawberry cultivar designated as ‘10.106-1’. Cultivar ‘10.106-1’ is typical of day-neutral strawberry cultivars and produces fruit regardless of day length when treated appropriately in arid, subtropical climates.

The strawberry cultivar ‘10.106-1’ originated from a cross between two unreleased germplasm accessions ‘05.206-5’ and ‘07.92-3’. The cultivar has been asexually reproduced by runners. The properties of this cultivar were found to be transmissible by such asexual reproduction. The cultivar is stable and reproduces true to type in successive generations of asexual reproduction.

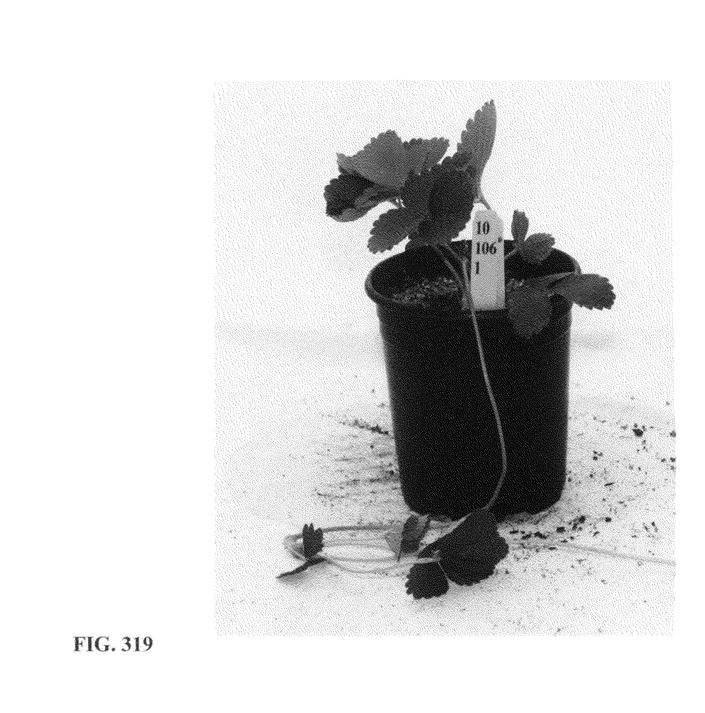

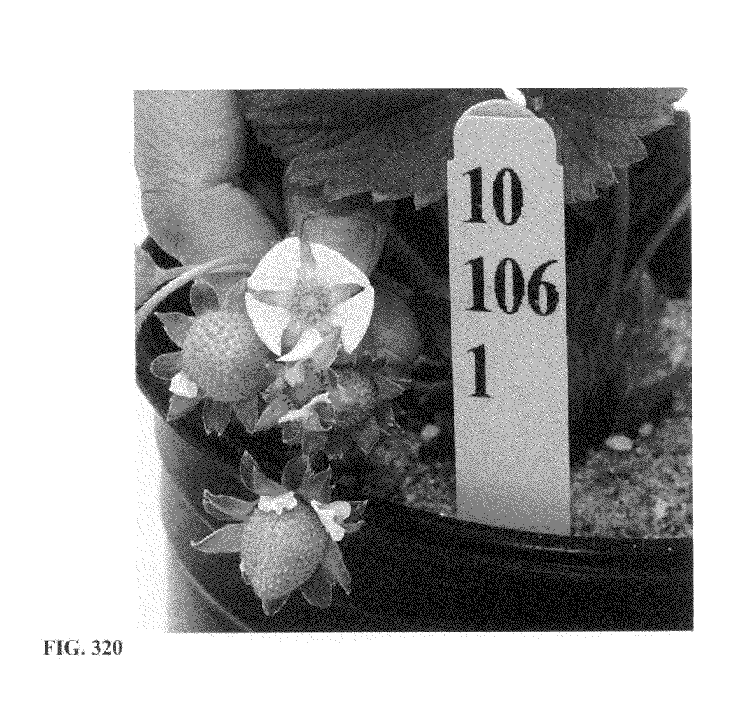

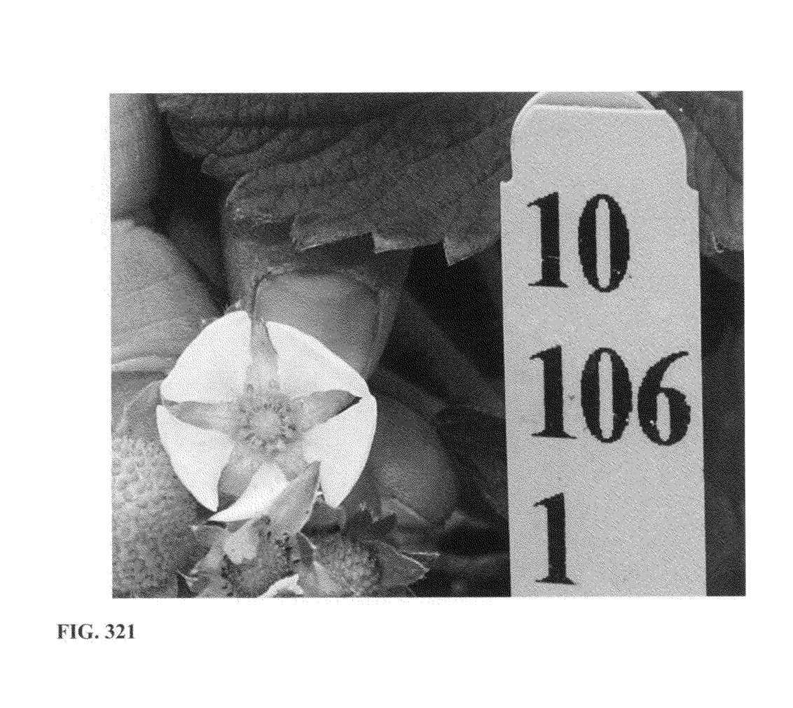

Cultivar ‘10.106-1’ has distinct disease/pathogen resistance, yield, appearance, size, and firmness. The plant of cultivar ‘10.106-1’ is characterized and distinguished in FIG. 319, the fruit of cultivar ‘10.106-1’ is characterized and distinguished in FIG. 320, and the flower of cultivar ‘10.106-1’ is characterized and distinguished in FIG. 321.

The following detailed description of cultivar ‘10.106-1’ is based on observations taken of plants and fruits (see FIGS. 319 and 320).

Certain characteristics of cultivar ‘10.106-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 107 provides data that characterizes the yield, appearance, size, and firmness of cultivar ‘10.106-1’.

TABLE 107

| Cultivar | Yield | App | Size | Firm |
|---|---|---|---|---|
| 10.106-1 | 2230 | 3.8 | 30.5 | 10.6 |

### Cultivar ‘10.106-8’

In certain aspects, the present invention relates to a new and distinctive day-neutral type strawberry cultivar designated as ‘10.106-8’. Cultivar ‘10.106-8’ is typical of day-neutral strawberry cultivars and produces fruit regardless of day length when treated appropriately in arid, subtropical climates.

The strawberry cultivar ‘10.106-8’ originated from a cross between two unreleased germplasm accessions ‘05.206-5’ and ‘07.92-3’. The cultivar has been asexually reproduced by runners. The properties of this cultivar were found to be transmissible by such asexual reproduction. The cultivar is stable and reproduces true to type in successive generations of asexual reproduction.

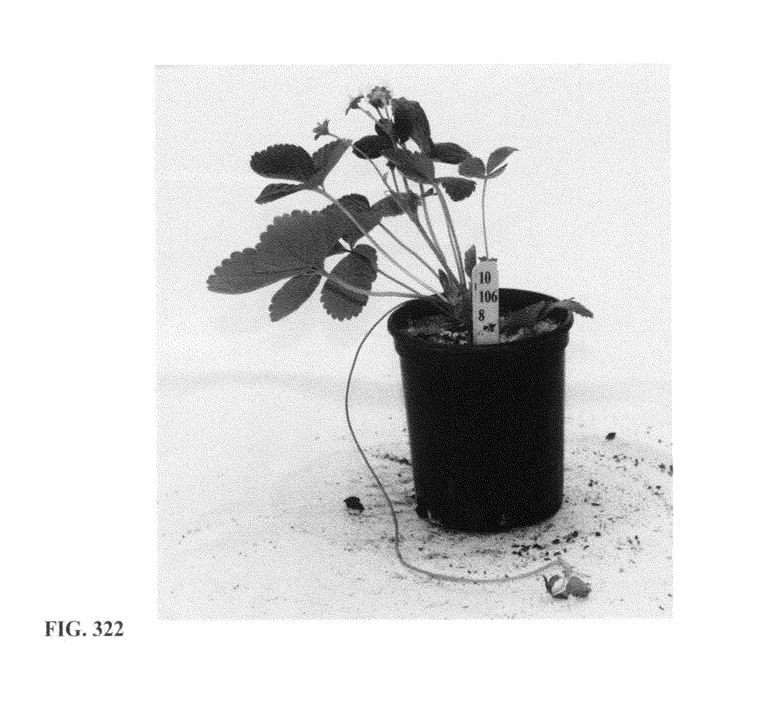

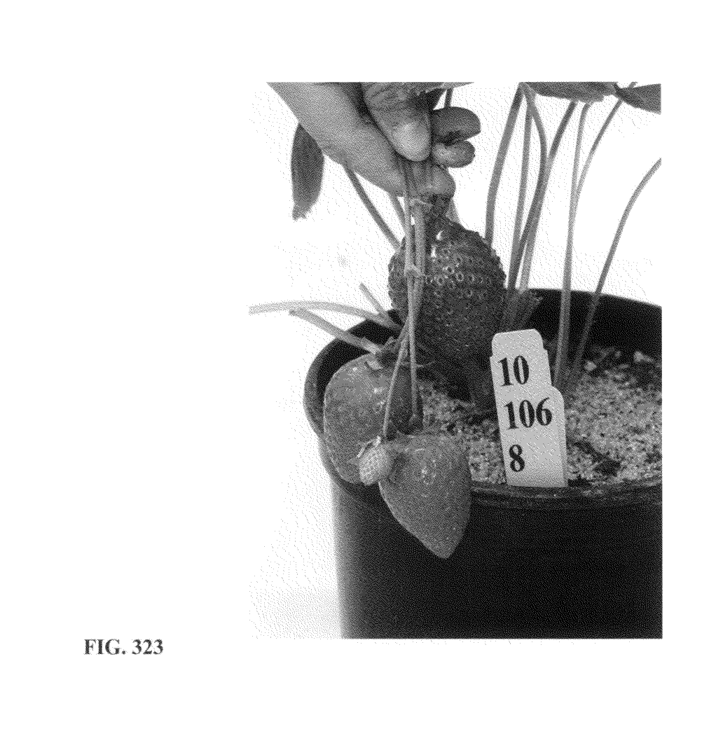

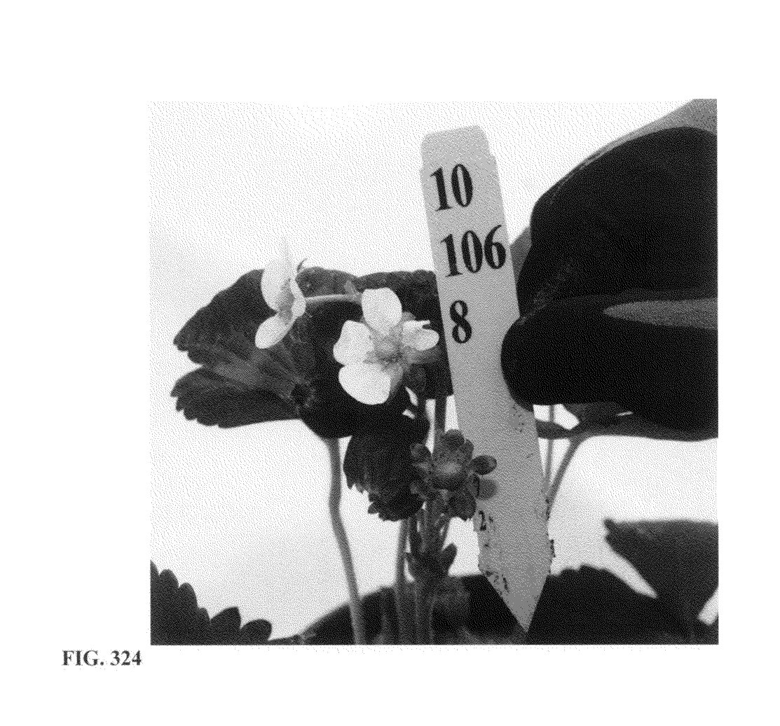

Cultivar ‘10.106-8’ has distinct disease/pathogen resistance, yield, appearance, size, and firmness. The plant of cultivar ‘10.106-8’ is characterized and distinguished in FIG. 322, the fruit of cultivar ‘10.106-8’ is characterized and distinguished in FIG. 323, and the flower of cultivar ‘10.106-8’ is characterized and distinguished in FIG. 324.

The following detailed description of cultivar ‘10.106-8’ is based on observations taken of plants and fruits (see FIGS. 322 and 323).

Certain characteristics of cultivar ‘10.106-8’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 108 provides data that characterizes the yield, appearance, size, and firmness of cultivar ‘10.106-8’.

TABLE 108

| Cultivar | Yield | App | Size | Firm |
|---|---|---|---|---|
| 10.106-8 | 2259 | 4.2 | 32.9 | 13.8 |

### Cultivar ‘10.116-1’

In certain aspects, the present invention relates to a new and distinctive day-neutral type strawberry cultivar designated as ‘10.116-1’. Cultivar ‘10.116-1’ is typical of day-neutral strawberry cultivars and produces fruit regardless of day length when treated appropriately in arid, subtropical climates.

The strawberry cultivar ‘10.116-1’ originated from a cross between two unreleased germplasm accessions ‘05.206-5’ and ‘05.148-2’. The cultivar has been asexually reproduced by runners. The properties of this cultivar were found to be transmissible by such asexual reproduction. The cultivar is stable and reproduces true to type in successive generations of asexual reproduction.

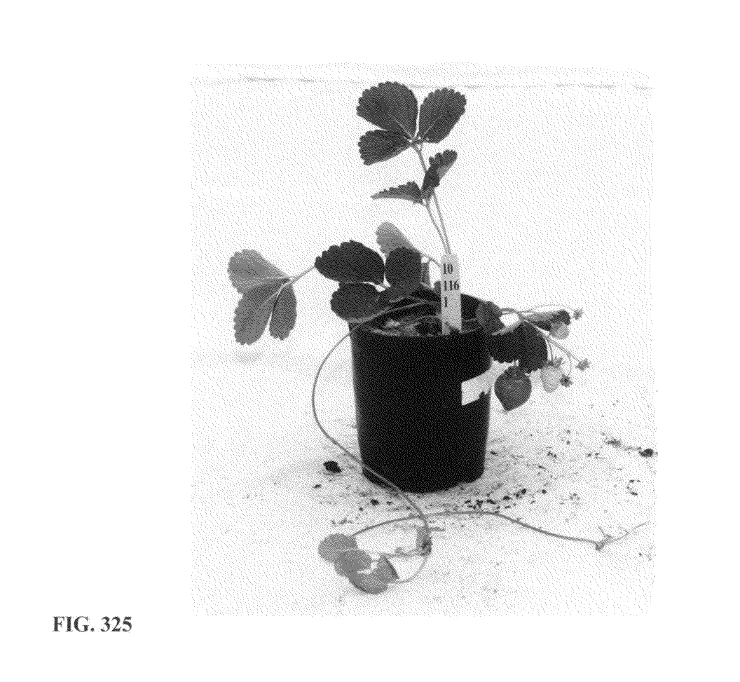

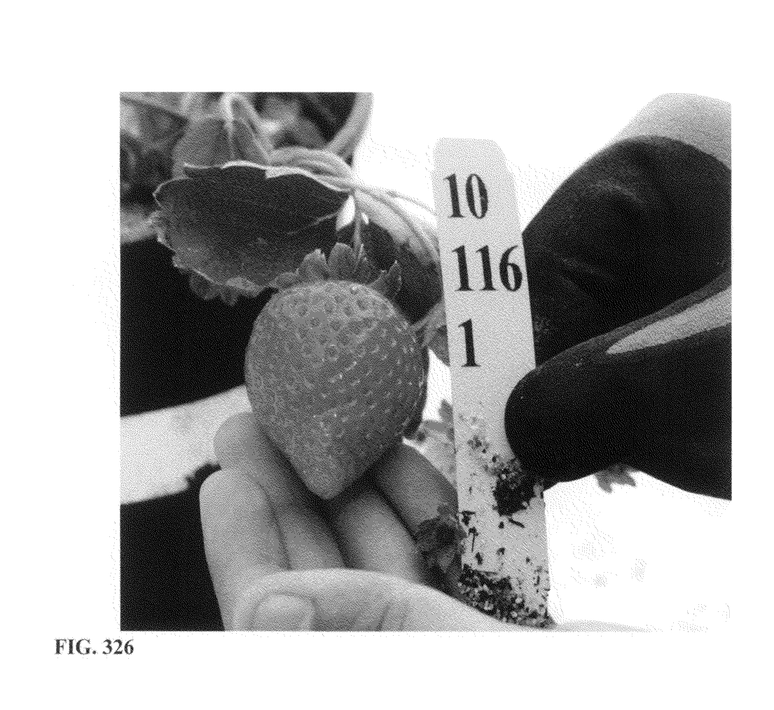

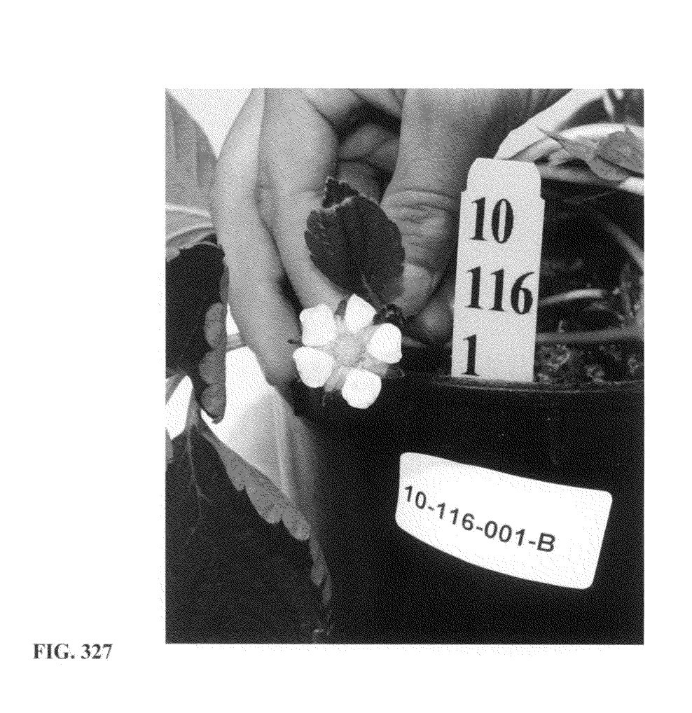

Cultivar ‘10.116-1’ has distinct disease/pathogen resistance, yield, appearance, size, and firmness. The plant of cultivar ‘10.116-1’ is characterized and distinguished in FIG. 325, the fruit of cultivar ‘10.116-1’ is characterized and distinguished in FIG. 326, and the flower of cultivar ‘10.116-1’ is characterized and distinguished in FIG. 327.

The following detailed description of cultivar ‘10.116-1’ is based on observations taken of plants and fruits (see FIGS. 325 and 326).

Certain characteristics of cultivar ‘10.116-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 109 provides data that characterizes the yield, appearance, size, and firmness of cultivar ‘10.116-1’.

TABLE 109

| Cultivar | Yield | App | Size | Firm |
|---|---|---|---|---|
| 10.116-1 | 2111 | 4.1 | 33.4 | 13.1 |

### Cultivar ‘10.122-3’

In certain aspects, the present invention relates to a new and distinctive day-neutral type strawberry cultivar designated as ‘10.122-3’. Cultivar ‘10.122-3’ is typical of day-neutral strawberry cultivars and produces fruit regardless of day length when treated appropriately in arid, subtropical climates.

The strawberry cultivar ‘10.122-3’ originated from a cross between two unreleased germplasm accessions ‘05.206-5’ and ‘05.197-2’. The cultivar has been asexually reproduced by runners. The properties of this cultivar were found to be transmissible by such asexual reproduction. The cultivar is stable and reproduces true to type in successive generations of asexual reproduction.

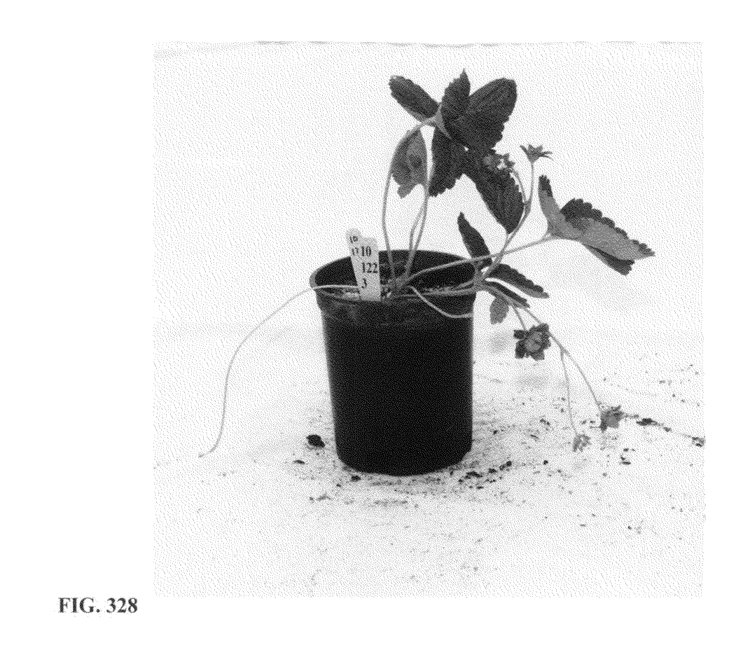

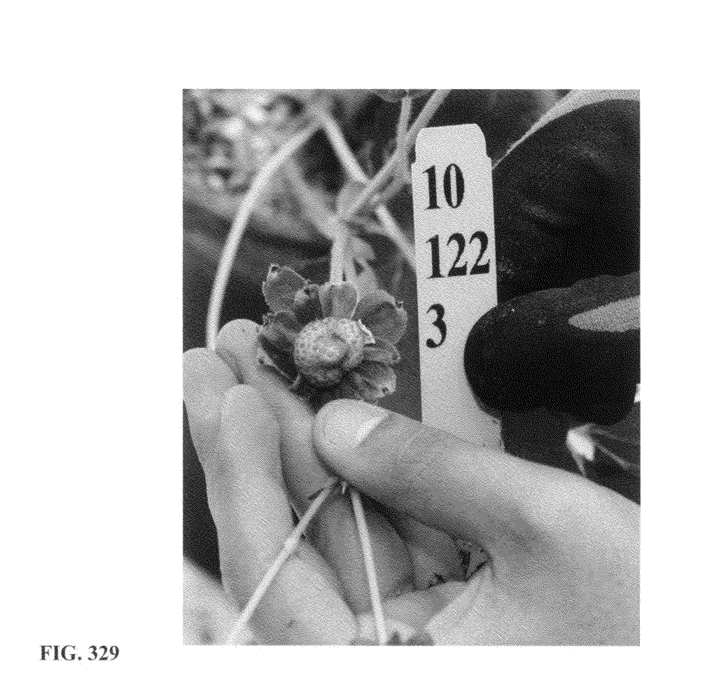

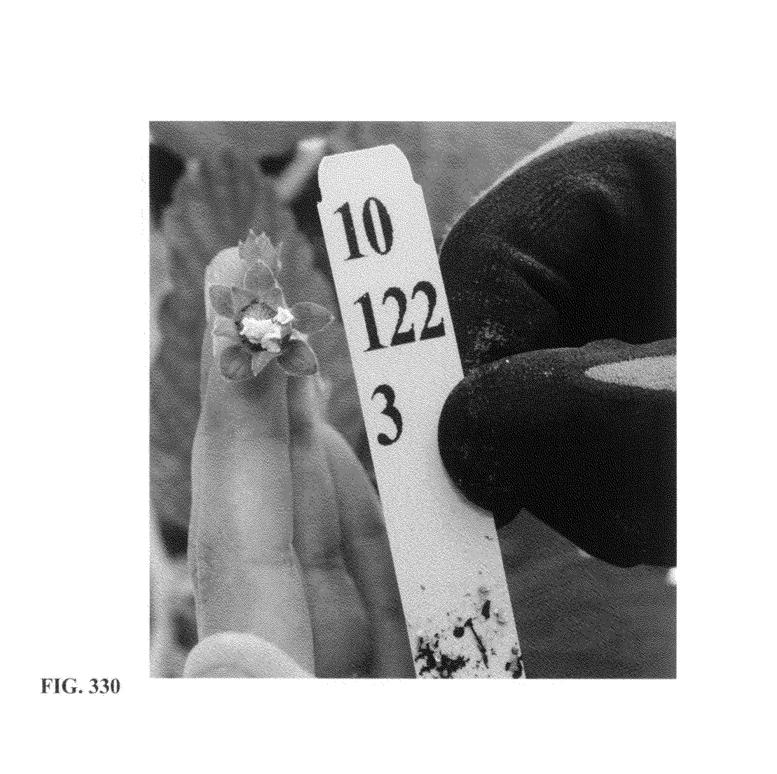

Cultivar ‘10.122-3’ has distinct disease/pathogen resistance, yield, appearance, size, and firmness. The plant of cultivar ‘10.122-3’ is characterized and distinguished in FIG. 328, the fruit of cultivar ‘10.122-3’ is characterized and distinguished in FIG. 329, and the flower of cultivar ‘10.122-3’ is characterized and distinguished in FIG. 330.

The following detailed description of cultivar ‘10.122-3’ is based on observations taken of plants and fruits (see FIGS. 328 and 329).

Certain characteristics of cultivar ‘10.122-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 110 provides data that characterizes the yield, appearance, size, and firmness of cultivar ‘10.122-3’.

TABLE 110

| Cultivar | Yield | App | Size | Firm |
|---|---|---|---|---|
| 10.122-3 | 2696 | 3.9 | 36.0 | 11.1 |

### Cultivar ‘10.122-6’

In certain aspects, the present invention relates to a new and distinctive day-neutral type strawberry cultivar designated as ‘10.122-6’. Cultivar ‘10.122-6’ is typical of day-neutral strawberry cultivars and produces fruit regardless of day length when treated appropriately in arid, subtropical climates.

The strawberry cultivar ‘10.122-6’ originated from a cross between two unreleased germplasm accessions ‘05.206-5’ and ‘05.197-2’. The cultivar has been asexually reproduced by runners. The properties of this cultivar were found to be transmissible by such asexual reproduction. The cultivar is stable and reproduces true to type in successive generations of asexual reproduction.

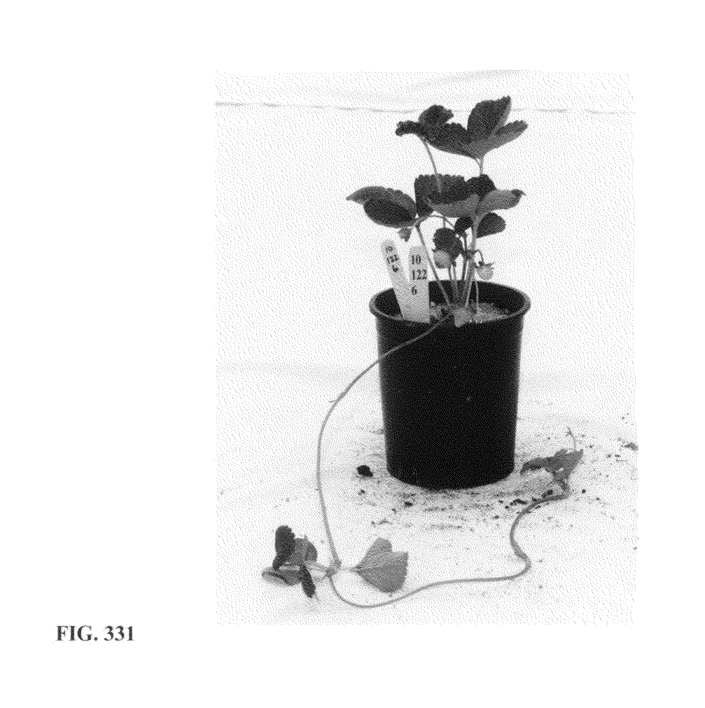

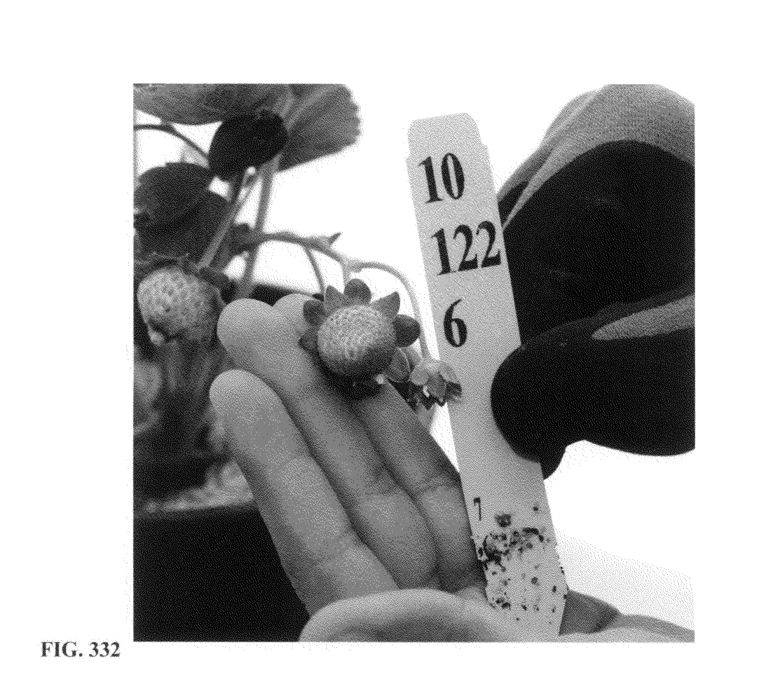

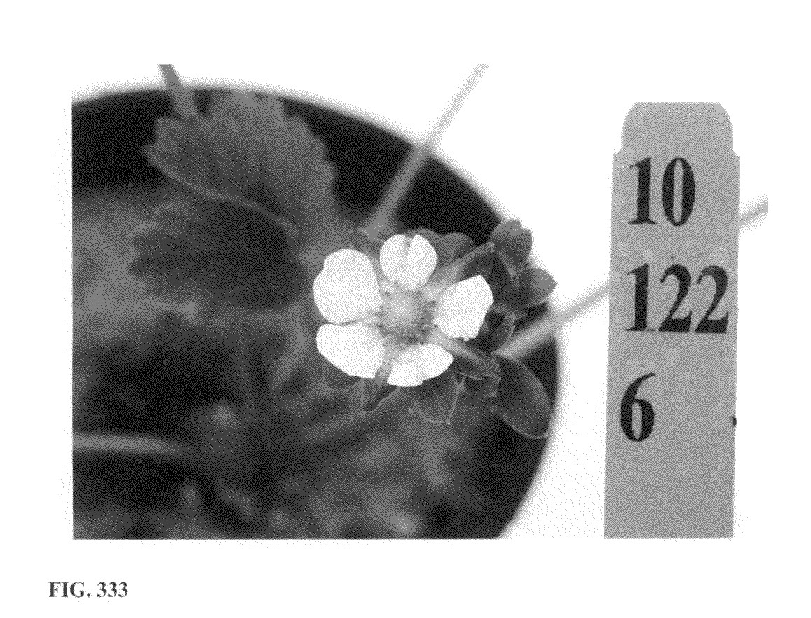

Cultivar ‘10.122-6’ has distinct disease/pathogen resistance, yield, appearance, size, and firmness. The plant of cultivar ‘10.122-6’ is characterized and distinguished in FIG. 331, the fruit of cultivar ‘10.122-6’ is characterized and distinguished in FIG. 332, and the flower of cultivar ‘10.122-6’ is characterized and distinguished in FIG. 333.

The following detailed description of cultivar ‘10.122-6’ is based on observations taken of plants and fruits (see FIGS. 331 and 332).

Certain characteristics of cultivar ‘10.122-6’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 111 provides data that characterizes the yield, appearance, size, and firmness of cultivar ‘10.122-6’.

TABLE 111

| Cultivar | Yield | App | Size | Firm |
|---|---|---|---|---|
| 10.122-6 | 2512 | 4.1 | 36.1 | 12.1 |

### Cultivar ‘10.124-2’

In certain aspects, the present invention relates to a new and distinctive short-day type strawberry cultivar designated as ‘10.124-2’. Cultivar ‘10.124-2’ is typical of short-day strawberry cultivars and produces fruit over an extended period when treated appropriately in arid, subtropical climates.

The strawberry cultivar ‘10.124-2’ originated from a cross between two unreleased germplasm accessions ‘06.51-609’ and ‘05.197-2’. The cultivar has been asexually reproduced by runners. The properties of this cultivar were found to be transmissible by such asexual reproduction. The cultivar is stable and reproduces true to type in successive generations of asexual reproduction.

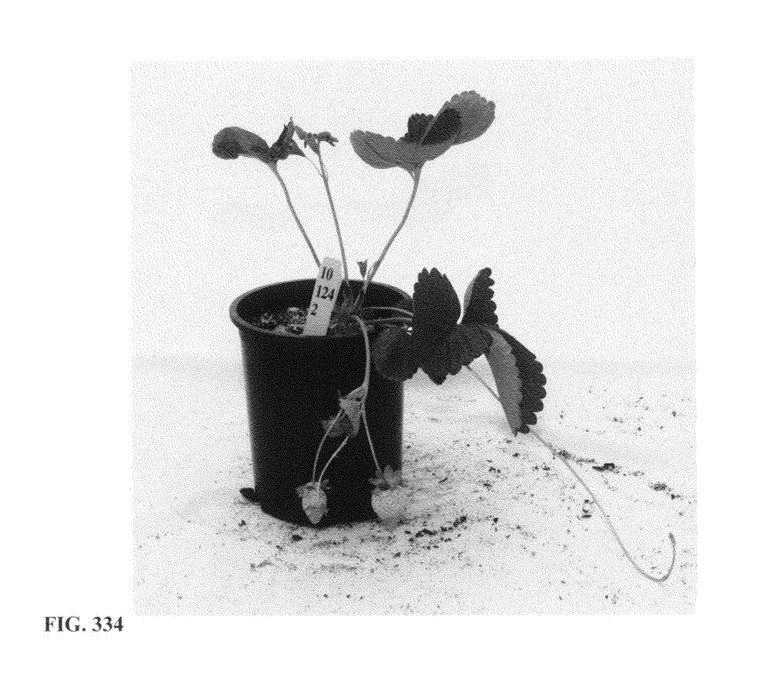

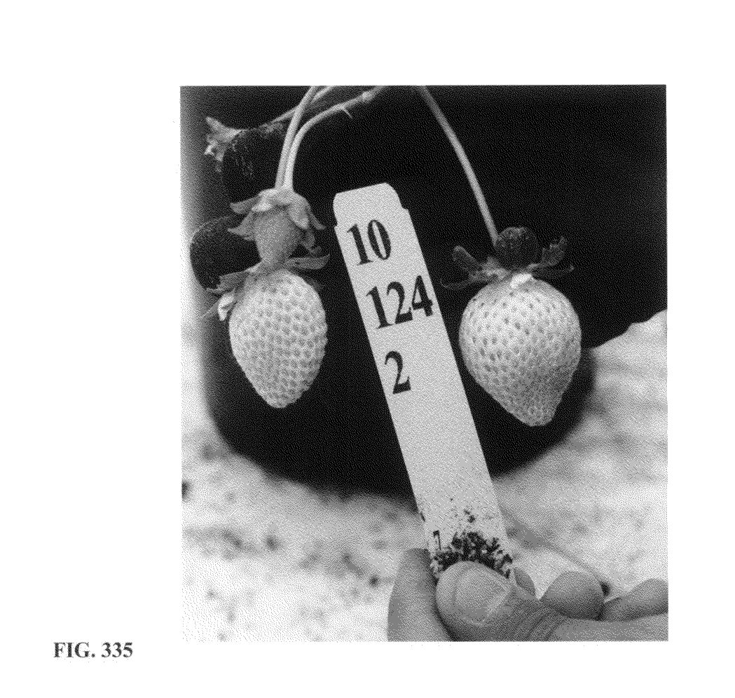

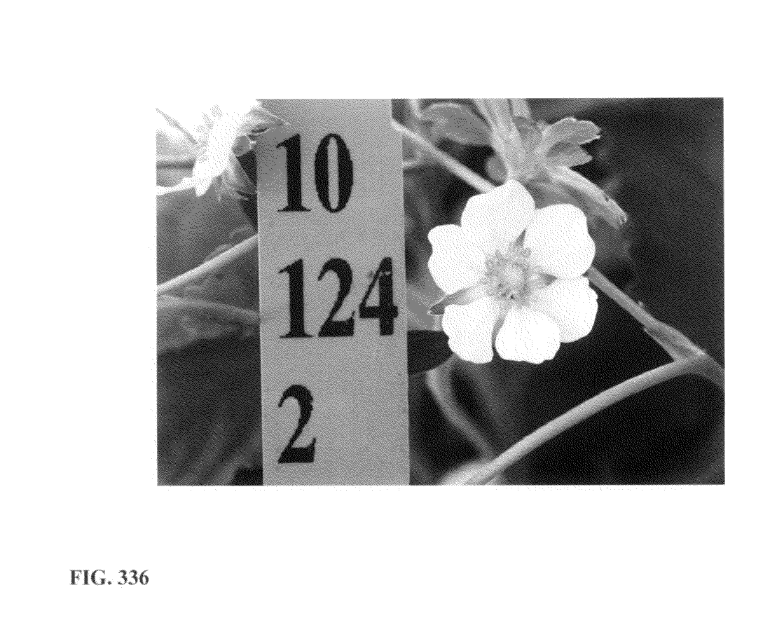

Cultivar ‘10.124-2’ has distinct disease/pathogen resistance, yield, appearance, size, and firmness. The plant of cultivar ‘10.124-2’ is characterized and distinguished in FIG. 334, the fruit of cultivar ‘10.124-2’ is characterized and distinguished in FIG. 335, and the flower of cultivar ‘10.124-2’ is characterized and distinguished in FIG. 336.

The following detailed description of cultivar ‘10.124-2’ is based on observations taken of plants and fruits (see FIGS. 334 and 335).

Certain characteristics of cultivar ‘10.124-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 112 provides data that characterizes the disease/pathogen resistance, yield, appearance, size, and firmness of cultivar ‘10.124-2’.

TABLE 112

| Cultivar | Vert | Fus | Yield | App | Size | Firm |
|---|---|---|---|---|---|---|
| 10.124-2 | 5.0 | 4.9 | 1900 | 4.0 | 37.0 | 11.4 |

### Cultivar ‘10.126-3’

In certain aspects, the present invention relates to a new and distinctive day-neutral type strawberry cultivar designated as ‘10.126-3’. Cultivar ‘10.126-3’ is typical of day-neutral strawberry cultivars and produces fruit regardless of day length when treated appropriately in arid, subtropical climates.

The strawberry cultivar ‘10.126-3’ originated from a cross between two unreleased germplasm accessions ‘07.120-3’ and ‘05.197-2’. The cultivar has been asexually reproduced by runners. The properties of this cultivar were found to be transmissible by such asexual reproduction. The cultivar is stable and reproduces true to type in successive generations of asexual reproduction.

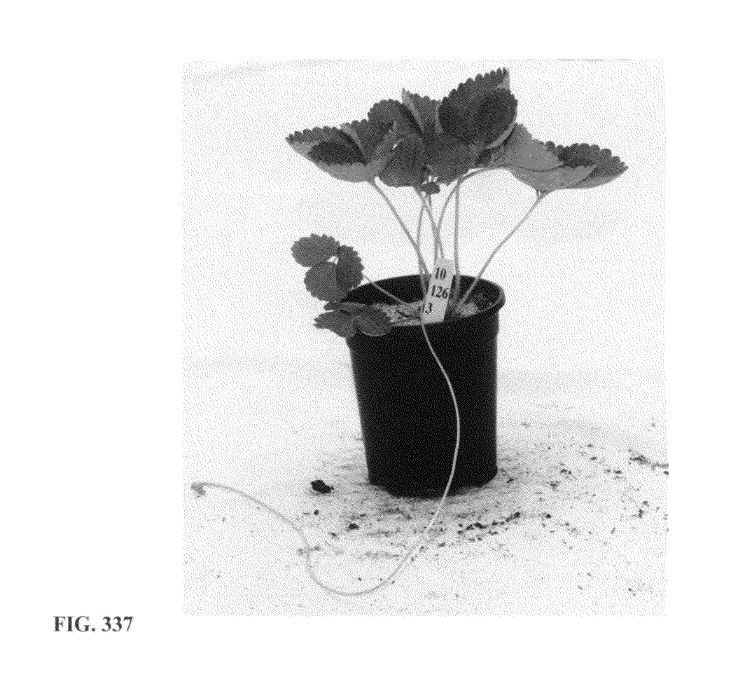

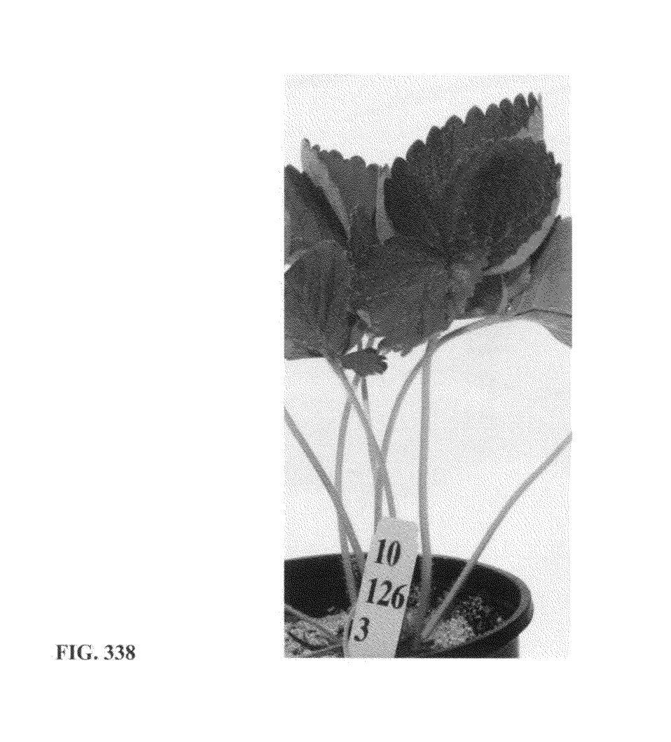

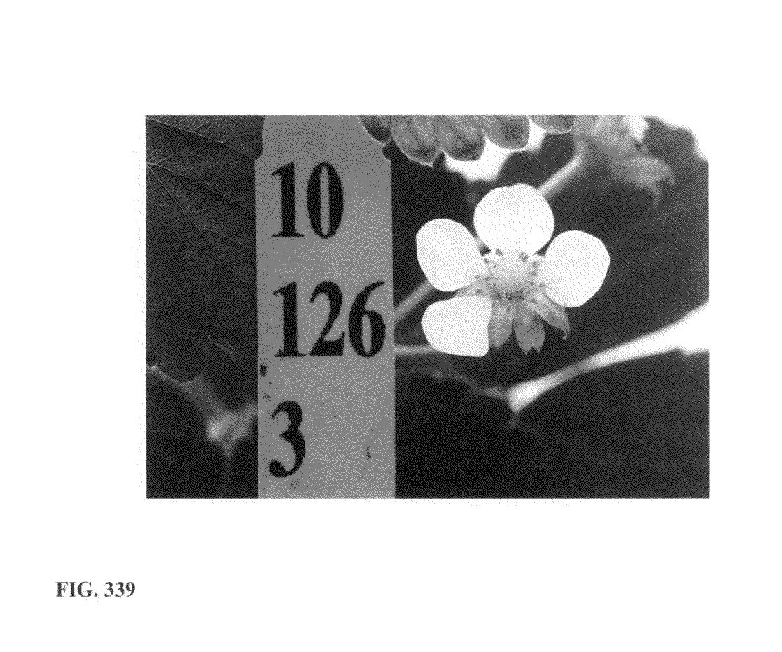

Cultivar ‘10.126-3’ has distinct disease/pathogen resistance, yield, appearance, size, and firmness. The plant of cultivar ‘10.126-3’ is characterized and distinguished in FIG. 337, a leaf and stem of cultivar ‘10.126-3’ is characterized and distinguished in FIG. 338, and the flower of cultivar ‘10.126-3’ is characterized and distinguished in FIG. 339.

The following detailed description of cultivar ‘10.126-3’ is based on observations taken of plants, leaves, and stems (see FIGS. 337 and 338).

Certain characteristics of cultivar ‘10.126-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 113 provides data that characterizes the yield, appearance, size, and firmness of cultivar ‘10.126-3’.

TABLE 113

| Cultivar | Yield | App | Size | Firm |
|---|---|---|---|---|
| 10.126-3 | 1832 | 3.9 | 40.8 | 11.2 |

### Cultivar ‘10.127-1’

In certain aspects, the present invention relates to a new and distinctive day-neutral type strawberry cultivar designated as ‘10.127-1’. Cultivar ‘10.127-1’ is typical of day-neutral strawberry cultivars and produces fruit regardless of day length when treated appropriately in arid, subtropical climates.

The strawberry cultivar ‘10.127-1’ originated from a cross between two unreleased germplasm accessions ‘05.108-7’ and ‘06.109-3’. The cultivar has been asexually reproduced by runners. The properties of this cultivar were found to be transmissible by such asexual reproduction. The cultivar is stable and reproduces true to type in successive generations of asexual reproduction.

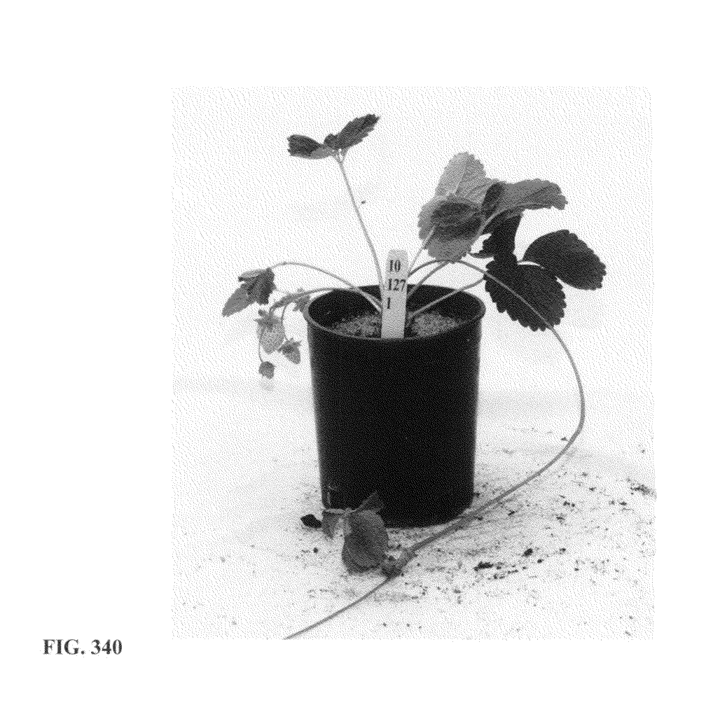

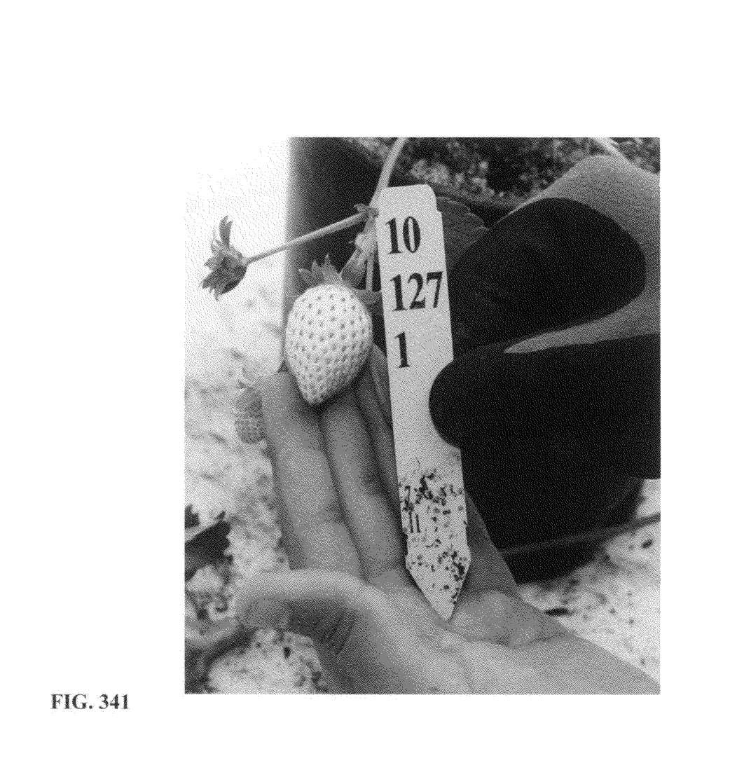

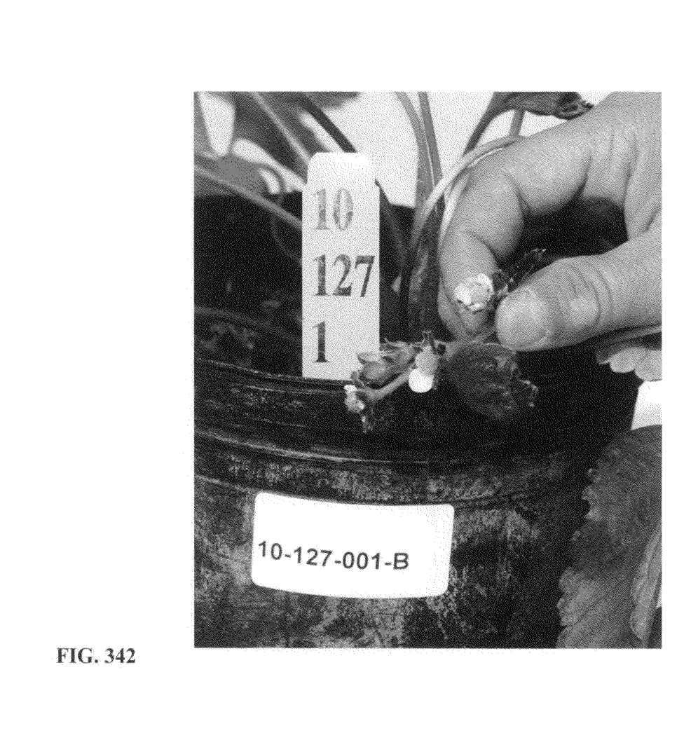

Cultivar ‘10.127-1’ has distinct disease/pathogen resistance, yield, appearance, size, and firmness. The plant of cultivar ‘10.127-1’ is characterized and distinguished in FIG. 340, the fruit of cultivar ‘10.127-1’ is characterized and distinguished in FIG. 341, and the flower of cultivar ‘10.127-1’ is characterized and distinguished in FIG. 342.

The following detailed description of cultivar ‘10.127-1’ is based on observations taken of plants and fruits (see FIGS. 340 and 341).

Certain characteristics of cultivar ‘10.127-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 114 provides data that characterizes the yield, appearance, size, and firmness of cultivar ‘10.127-1’.

TABLE 114

| Cultivar | Yield | App | Size | Firm |
|---|---|---|---|---|
| 10.127-1 | 2055 | 3.8 | 30.6 | 11.8 |

### Cultivar ‘10.134-4’

In certain aspects, the present invention relates to a new and distinctive day-neutral type strawberry cultivar designated as ‘10.134-4’. Cultivar ‘10.134-4’ is typical of day-neutral strawberry cultivars and produces fruit regardless of day length when treated appropriately in arid, subtropical climates.

The strawberry cultivar ‘10.134-4’ originated from a cross between two unreleased germplasm accessions ‘05.206-5’ and ‘06.132-1’. The cultivar has been asexually reproduced by runners. The properties of this cultivar were found to be transmissible by such asexual reproduction. The cultivar is stable and reproduces true to type in successive generations of asexual reproduction.

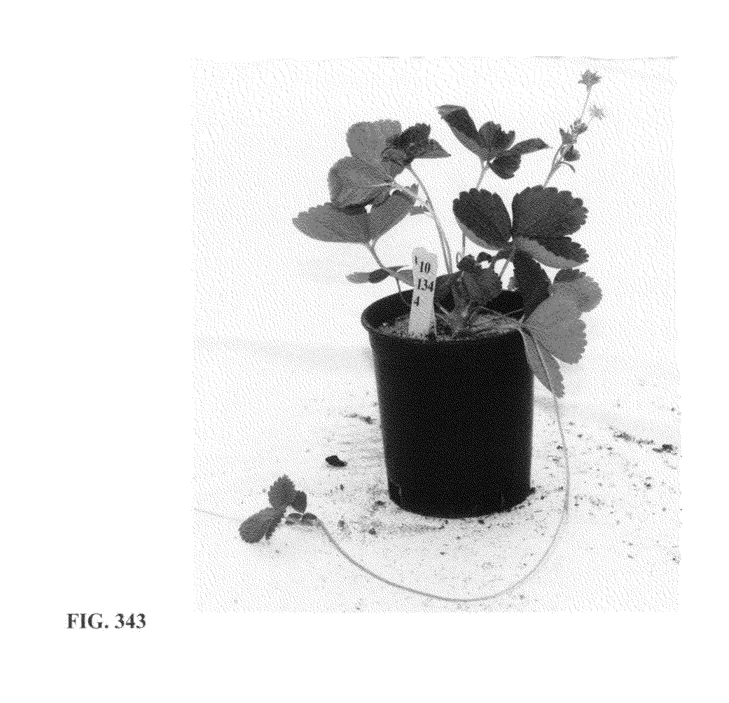

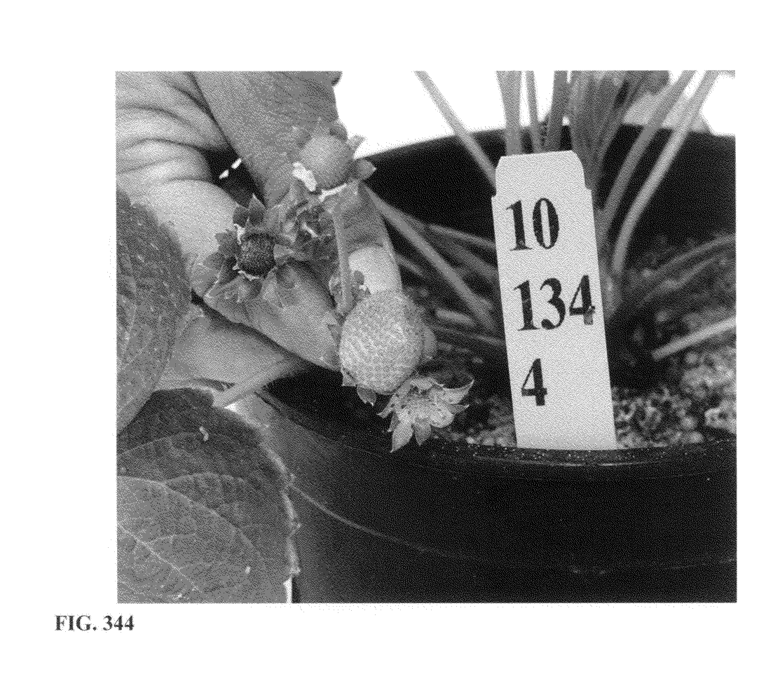

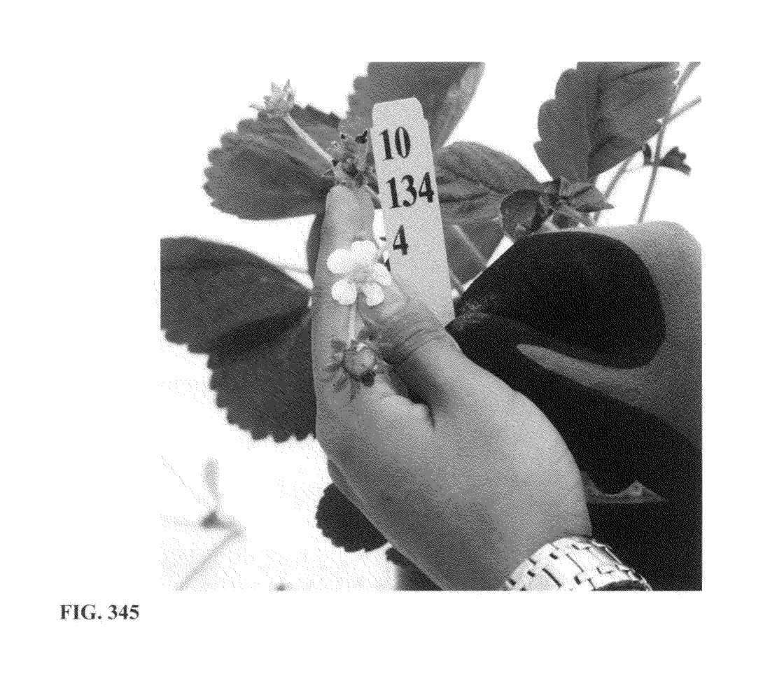

Cultivar ‘10.134-4’ has distinct disease/pathogen resistance, yield, appearance, size, and firmness. The plant of cultivar ‘10.134-4’ is characterized and distinguished in FIG. 343, the fruit of cultivar ‘10.134-4’ is characterized and distinguished in FIG. 344, and the flower of cultivar ‘10.134-4’ is characterized and distinguished in FIG. 345.

The following detailed description of cultivar ‘10.134-4’ is based on observations taken of plants and fruits (see FIGS. 343 and 344).

Certain characteristics of cultivar ‘10.134-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 115 provides data that characterizes the yield, appearance, size, and firmness of cultivar ‘10.134-4’.

TABLE 115

| Cultivar | Yield | App | Size | Firm |
|---|---|---|---|---|
| 10.134-4 | 2748 | 3.8 | 32.8 | 10.4 |

### Cultivar ‘10.136-4’

In certain aspects, the present invention relates to a new and distinctive short-day type strawberry cultivar designated as ‘10.136-4’. Cultivar ‘10.136-4’ is typical of short-day strawberry cultivars and produces fruit over an extended period when treated appropriately in arid, subtropical climates.

The strawberry cultivar ‘10.136-4’ originated from a cross between two unreleased germplasm accessions ‘06.51-609’ and ‘06.132-1’. The cultivar has been asexually reproduced by runners. The properties of this cultivar were found to be transmissible by such asexual reproduction. The cultivar is stable and reproduces true to type in successive generations of asexual reproduction.

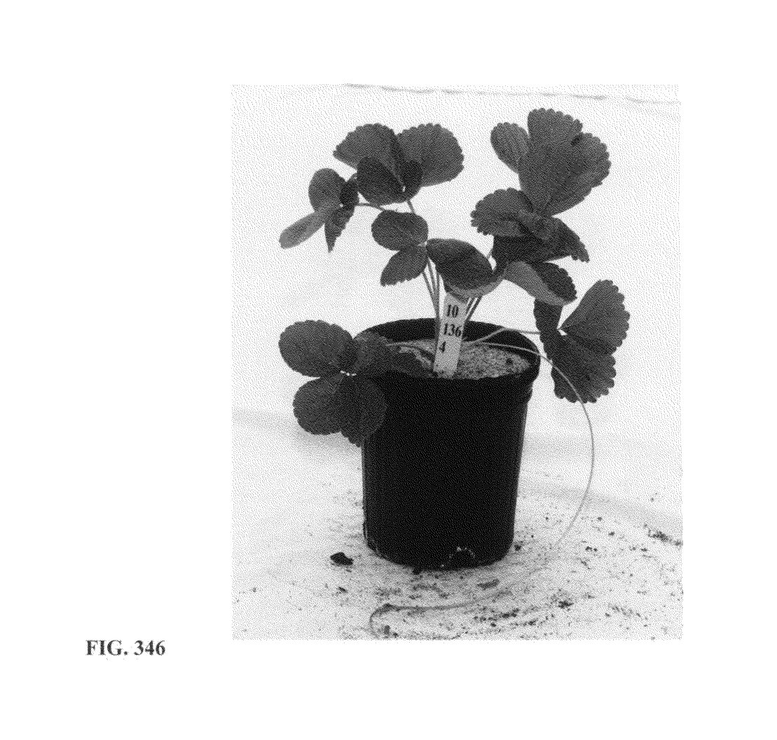

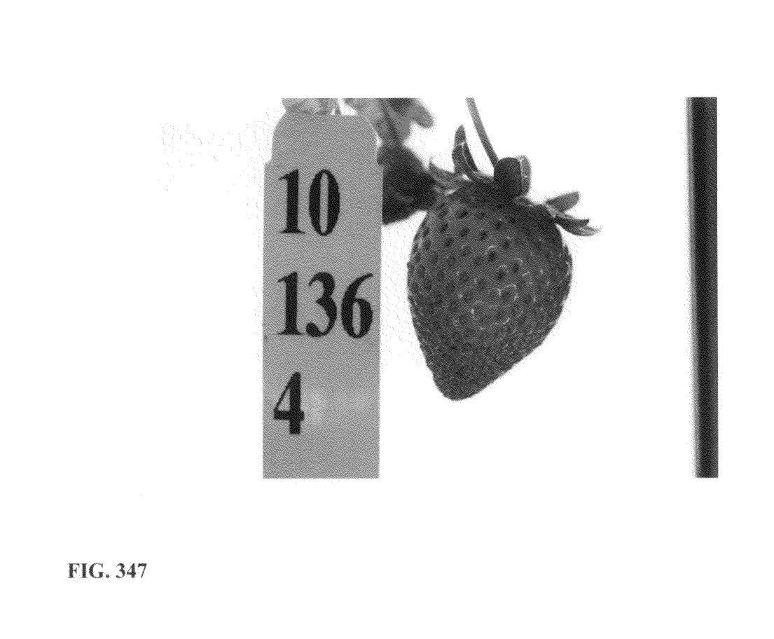

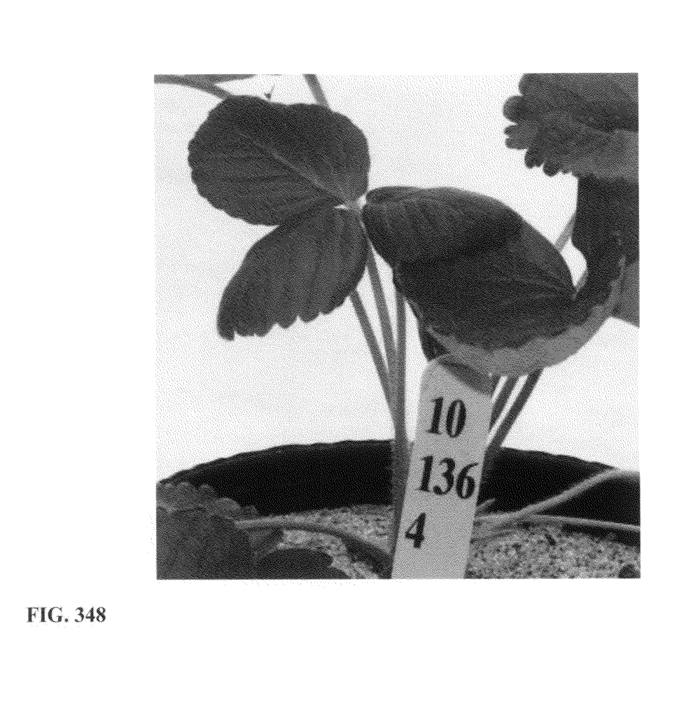

Cultivar ‘10.136-4’ has distinct disease/pathogen resistance, yield, appearance, size, and firmness. The plant of cultivar ‘10.136-4’ is characterized and distinguished in FIG. 346, the fruit of cultivar ‘10.136-4’ is characterized and distinguished in FIG. 347, and a leaf and stem of cultivar ‘10.136-4’ is characterized and distinguished in FIG. 348.

The following detailed description of cultivar ‘10.136-4’ is based on observations taken of plants and fruits (see FIGS. 346 and 347).

Certain characteristics of cultivar ‘10.136-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 116 provides data that characterizes the yield, appearance, size, and firmness of cultivar ‘10.136-4’.

TABLE 116

| Cultivar | Yield | App | Size | Firm |
|---|---|---|---|---|
| 10.136-4 | 1931 | 3.8 | 34.0 | 11.4 |

### Cultivar ‘10.137-5’

In certain aspects, the present invention relates to a new and distinctive short-day type strawberry cultivar designated as ‘10.137-5’. Cultivar ‘10.137-5’ is typical of short-day strawberry cultivars and produces fruit over an extended period when treated appropriately in arid, subtropical climates.

The strawberry cultivar ‘10.137-5’ originated from a cross between two unreleased germplasm accessions ‘06.102-2’ and ‘06.132-1’. The cultivar has been asexually reproduced by runners. The properties of this cultivar were found to be transmissible by such asexual reproduction. The cultivar is stable and reproduces true to type in successive generations of asexual reproduction.

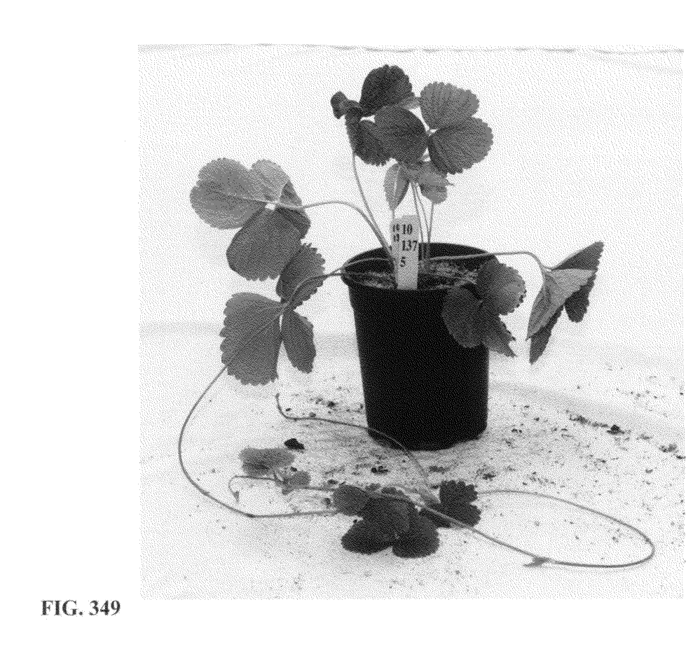

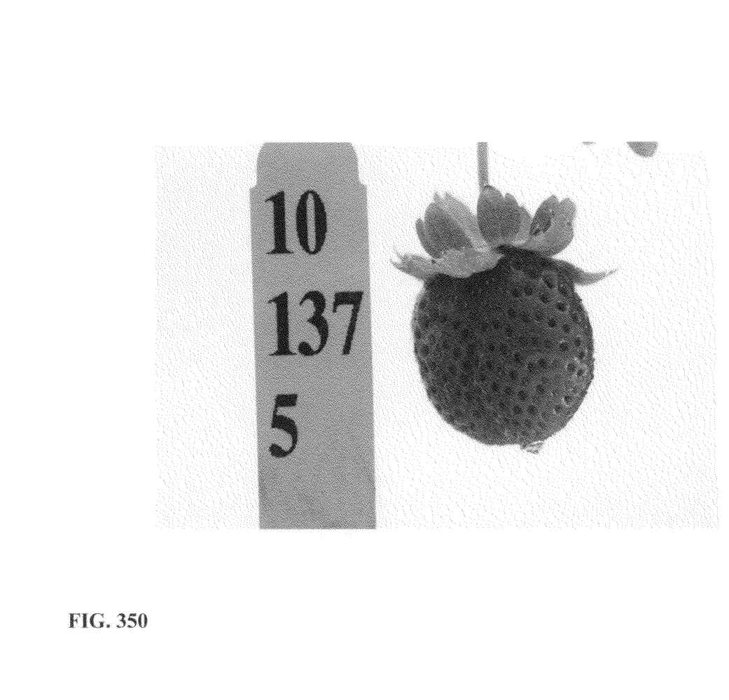

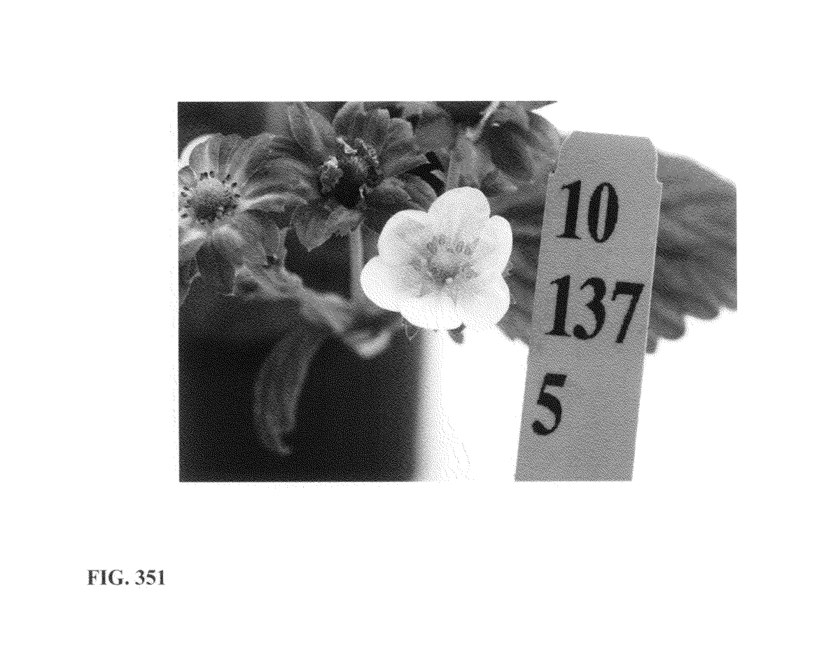

Cultivar ‘10.137-5’ has distinct disease/pathogen resistance, yield, appearance, size, and firmness. The plant of cultivar ‘10.137-5’ is characterized and distinguished in FIG. 349, the fruit of cultivar ‘10.137-5’ is characterized and distinguished in FIG. 350, and the flower of cultivar ‘10.137-5’ is characterized and distinguished in FIG. 351.

The following detailed description of cultivar ‘10.137-5’ is based on observations taken of plants and fruits (see FIGS. 349 and 350).

Certain characteristics of cultivar ‘10.137-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 117 provides data that characterizes the yield, appearance, size, and firmness of cultivar ‘10.137-5’.

TABLE 117

| Cultivar | Yield | App | Size | Firm |
|---|---|---|---|---|
| 10.137-5 | 2038 | 3.9 | 33.0 | 11.5 |

### Cultivar ‘10.140-1’

In certain aspects, the present invention relates to a new and distinctive day-neutral type strawberry cultivar designated as ‘10.140-1’. Cultivar ‘10.140-1’ is typical of day-neutral strawberry cultivars and produces fruit regardless of day length when treated appropriately in arid, subtropical climates.

The strawberry cultivar ‘10.140-1’ originated from a cross between two unreleased germplasm accessions ‘05.206-5’ and ‘07.43-1’. The cultivar has been asexually reproduced by runners. The properties of this cultivar were found to be transmissible by such asexual reproduction. The cultivar is stable and reproduces true to type in successive generations of asexual reproduction.

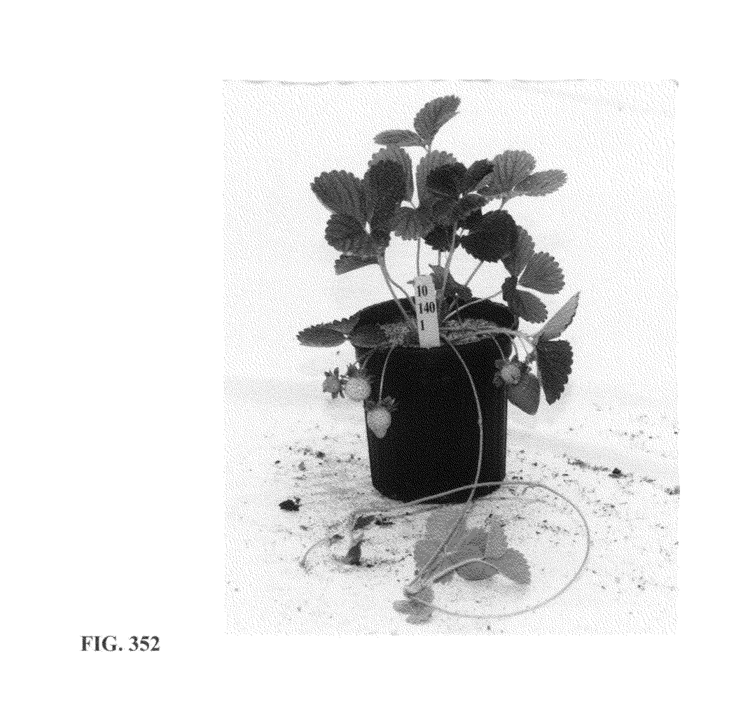

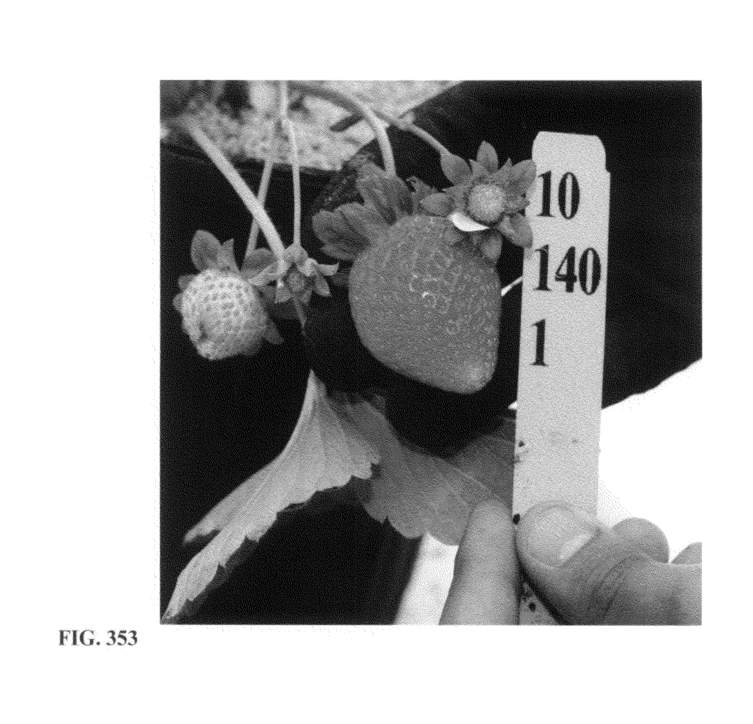

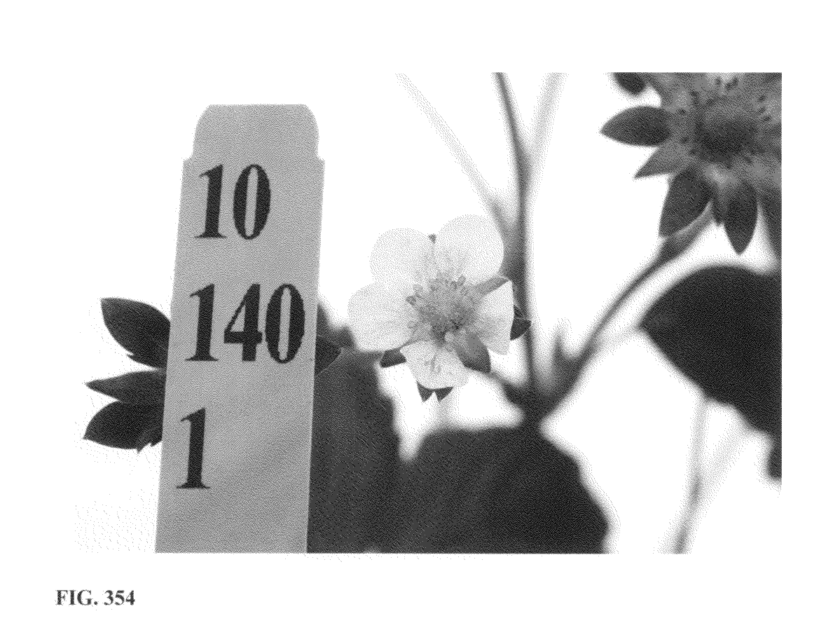

Cultivar ‘10.140-1’ has distinct disease/pathogen resistance, yield, appearance, size, and firmness. The plant of cultivar ‘10.140-1’ is characterized and distinguished in FIG. 352, the fruit of cultivar ‘10.140-1’ is characterized and distinguished in FIG. 353, and the flower of cultivar ‘10.140-1’ is characterized and distinguished in FIG. 354.

The following detailed description of cultivar ‘10.140-1’ is based on observations taken of plants and fruits (see FIGS. 352 and 353).

Certain characteristics of cultivar ‘10.140-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 118 provides data that characterizes the disease/pathogen resistance, yield, appearance, size, and firmness of cultivar ‘10.140-1’.

TABLE 118

| Cultivar | Vert | Fus | Yield | App | Size | Firm |
|---|---|---|---|---|---|---|
| 10.140-1 | 3.2 | 5.0 | 198 | 4.1 | 31.2 | 15.2 |

### Cultivar ‘10.141-1’

In certain aspects, the present invention relates to a new and distinctive short-day type strawberry cultivar designated as ‘10.141-1’. Cultivar ‘10.141-1’ is typical of short-day strawberry cultivars and produces fruit over an extended period when treated appropriately in arid, subtropical climates.

The strawberry cultivar ‘10.141-1’ originated from a cross between two unreleased germplasm accessions ‘06.43-603’ and ‘07.43-1’. The cultivar has been asexually reproduced by runners. The properties of this cultivar were found to be transmissible by such asexual reproduction. The cultivar is stable and reproduces true to type in successive generations of asexual reproduction.

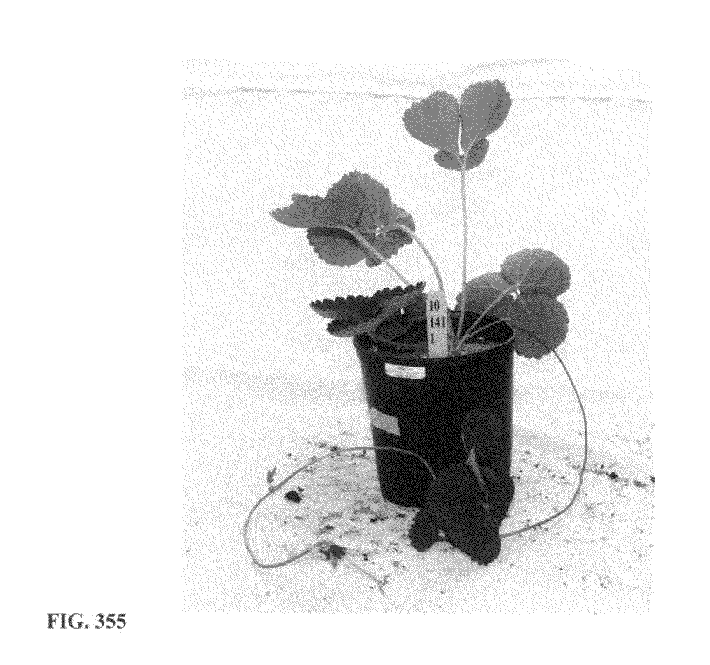

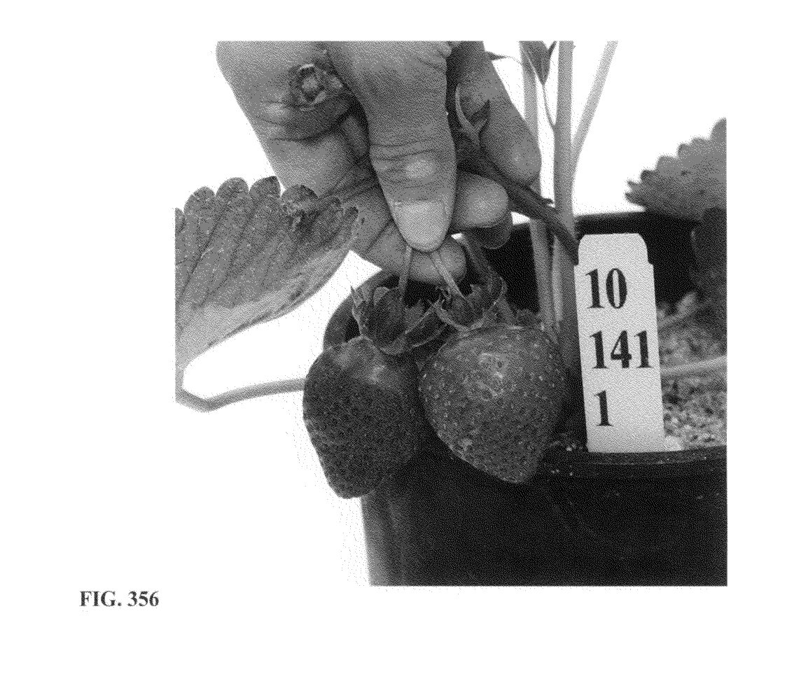

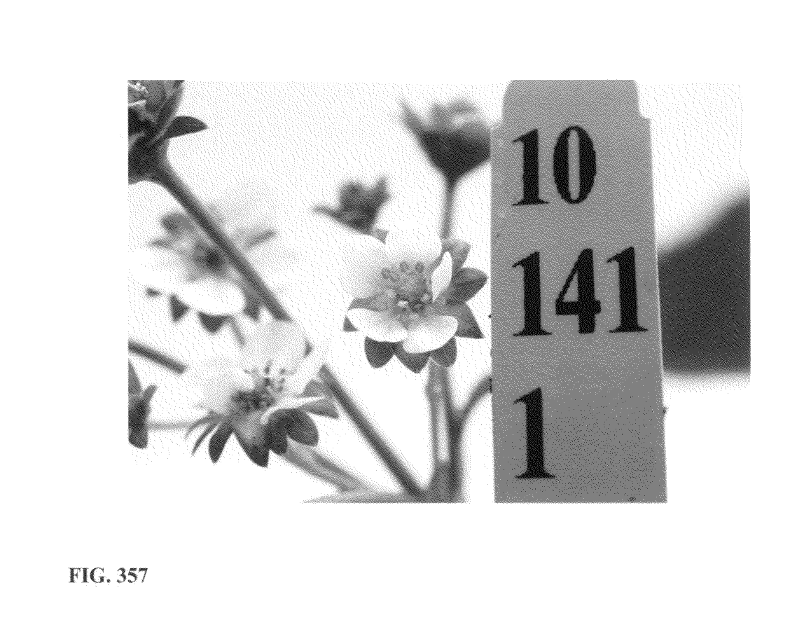

Cultivar ‘10.141-1’ has distinct disease/pathogen resistance, yield, appearance, size, and firmness. The plant of cultivar ‘10.141-1’ is characterized and distinguished in FIG. 355, the fruit of cultivar ‘10.141-1’ is characterized and distinguished in FIG. 356, and the flower of cultivar ‘10.141-1’ is characterized and distinguished in FIG. 357.

The following detailed description of cultivar ‘10.141-1’ is based on observations taken of plants and fruits (see FIGS. 355 and 356).

Certain characteristics of cultivar ‘10.141-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 119 provides data that characterizes the disease/pathogen resistance, yield, appearance, size, and firmness of cultivar ‘10.141-1’.

TABLE 119

| Cultivar | Vert | Fus | Yield | App | Size | Firm |
|---|---|---|---|---|---|---|
| 10.141-1 | 4.0 | 4.9 | 1597 | 3.9 | 34.5 | 12.6 |

### Cultivar ‘10.143-1’

In certain aspects, the present invention relates to a new and distinctive day-neutral type strawberry cultivar designated as ‘10.143-1’. Cultivar ‘10.143-1’ is typical of day-neutral strawberry cultivars and produces fruit regardless of day length when treated appropriately in arid, subtropical climates.

The strawberry cultivar ‘10.143-1’ originated from a cross between two unreleased germplasm accessions ‘06.102-2’ and ‘07.43-1’. The cultivar has been asexually reproduced by runners. The properties of this cultivar were found to be transmissible by such asexual reproduction. The cultivar is stable and reproduces true to type in successive generations of asexual reproduction.

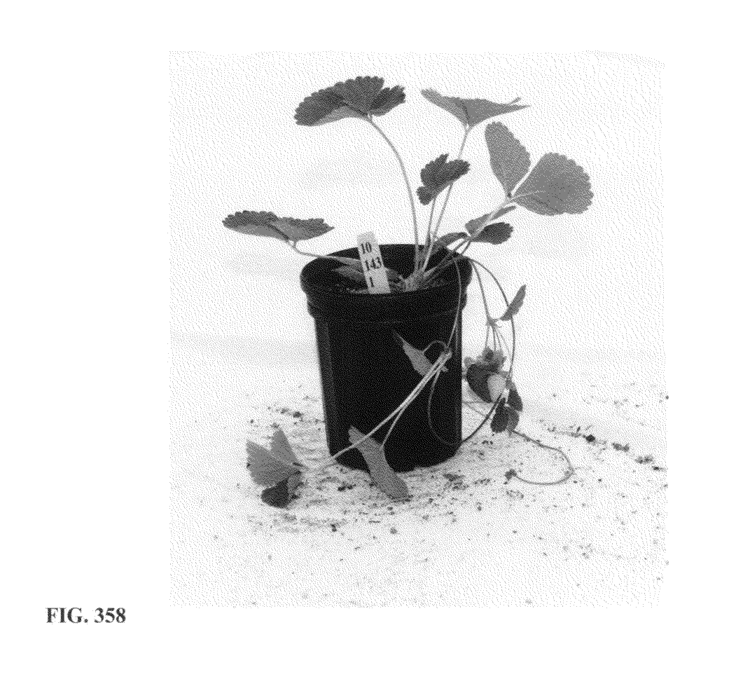

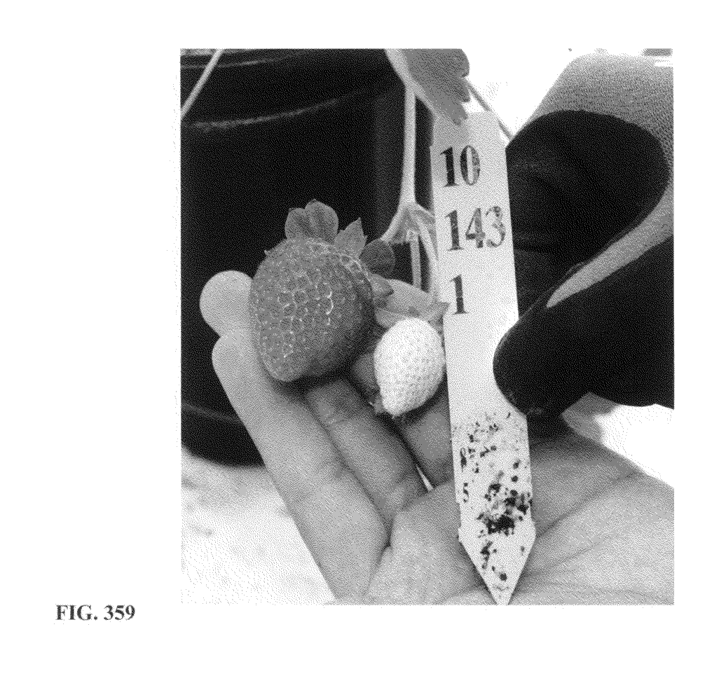

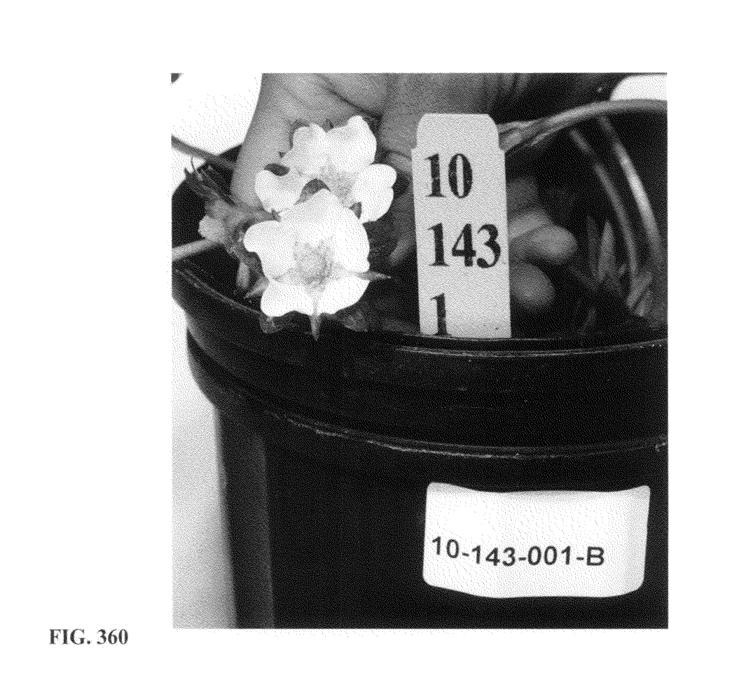

Cultivar ‘10.143-1’ has distinct disease/pathogen resistance, yield, appearance, size, and firmness. The plant of cultivar ‘10.143-1’ is characterized and distinguished in FIG. 358, the fruit of cultivar ‘10.143-1’ is characterized and distinguished in FIG. 359, and the flower of cultivar ‘10.143-1’ is characterized and distinguished in FIG. 360.

The following detailed description of cultivar ‘10.143-1’ is based on observations taken of plants and fruits (see FIGS. 358 and 359).

Certain characteristics of cultivar ‘10.143-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 120 provides data that characterizes the yield, appearance, size, and firmness of cultivar ‘10.143-1’.

TABLE 120

| Cultivar | Yield | App | Size | Firm |
|---|---|---|---|---|
| 10.143-1 | 1999 | 4.0 | 30.0 | 13.9 |

### Cultivar ‘10.144-1’

In certain aspects, the present invention relates to a new and distinctive short-day type strawberry cultivar designated as ‘10.144-1’. Cultivar ‘10.144-1’ is typical of short-day strawberry cultivars and produces fruit over an extended period when treated appropriately in arid, subtropical climates.

The strawberry cultivar ‘10.144-1’ originated from a cross between two unreleased germplasm accessions ‘07.120-3’ and ‘07.43-1’. The cultivar has been asexually reproduced by runners. The properties of this cultivar were found to be transmissible by such asexual reproduction. The cultivar is stable and reproduces true to type in successive generations of asexual reproduction.

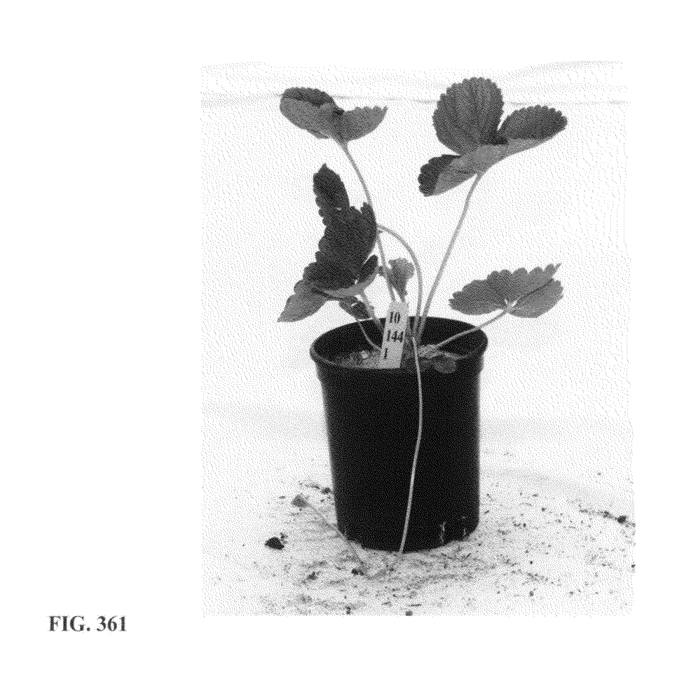

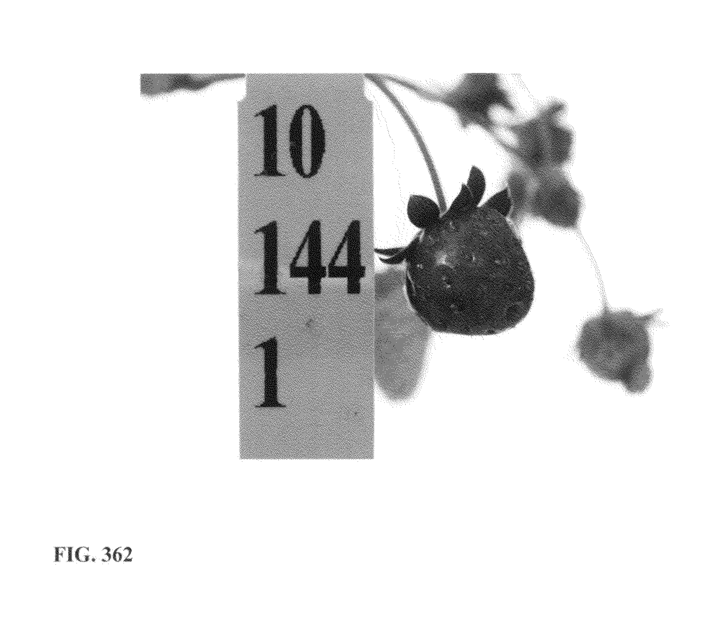

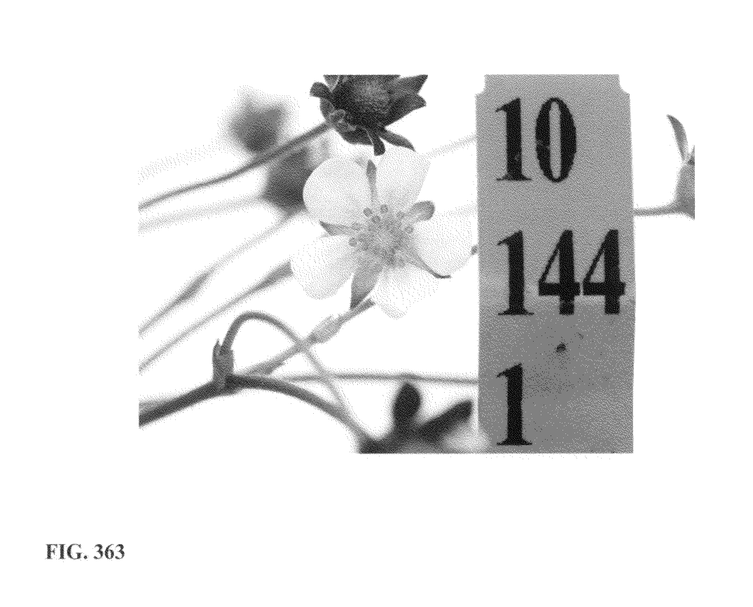

Cultivar ‘10.144-1’ has distinct disease/pathogen resistance, yield, appearance, size, and firmness. The plant of cultivar ‘10.144-1’ is characterized and distinguished in FIG. 361, the fruit of cultivar ‘10.144-1’ is characterized and distinguished in FIG. 362, and the flower of cultivar ‘10.144-1’ is characterized and distinguished in FIG. 363.

The following detailed description of cultivar ‘10.144-1’ is based on observations taken of plants and fruits (see FIGS. 361 and 362).

Certain characteristics of cultivar ‘10.144-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 121 provides data that characterizes the disease/pathogen resistance, yield, appearance, size, and firmness of cultivar ‘10.144-1’.

TABLE 121

| Cultivar | Vert | Fus | Yield | App | Size | Firm |
|---|---|---|---|---|---|---|
| 10.144-1 | 3.5 | 5.0 | 1934 | 4.0 | 34.4 | 10.0 |

### Cultivar ‘10.144-2’

In certain aspects, the present invention relates to a new and distinctive short-day type strawberry cultivar designated as ‘10.144-2’. Cultivar ‘10.144-2’ is typical of short-day strawberry cultivars and produces fruit over an extended period when treated appropriately in arid, subtropical climates.

The strawberry cultivar ‘10.144-2’ originated from a cross between two unreleased germplasm accessions ‘07.120-3’ and ‘07.43-1’. The cultivar has been asexually reproduced by runners. The properties of this cultivar were found to be transmissible by such asexual reproduction. The cultivar is stable and reproduces true to type in successive generations of asexual reproduction.

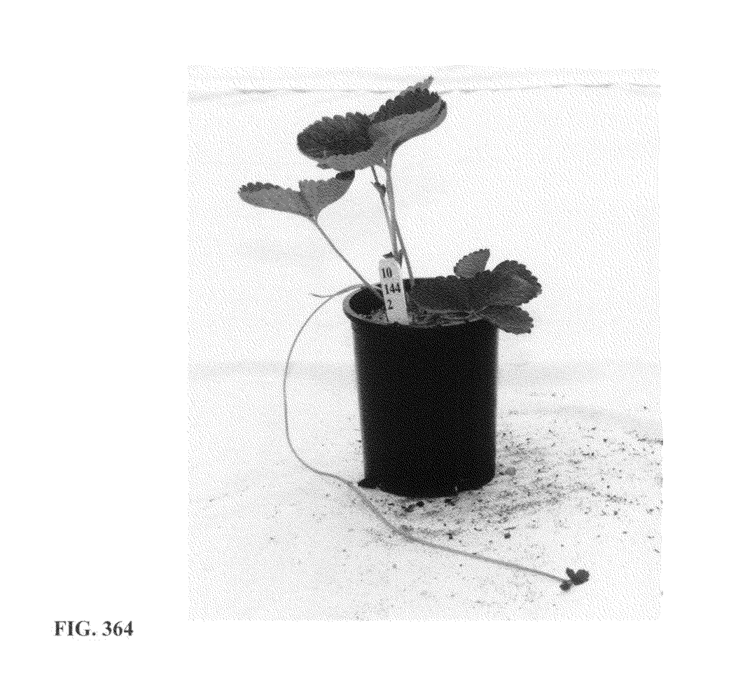

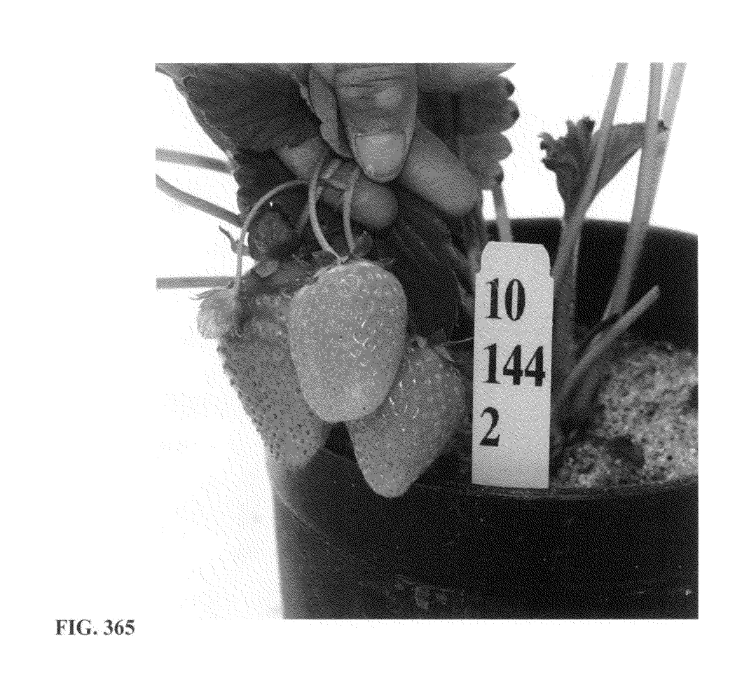

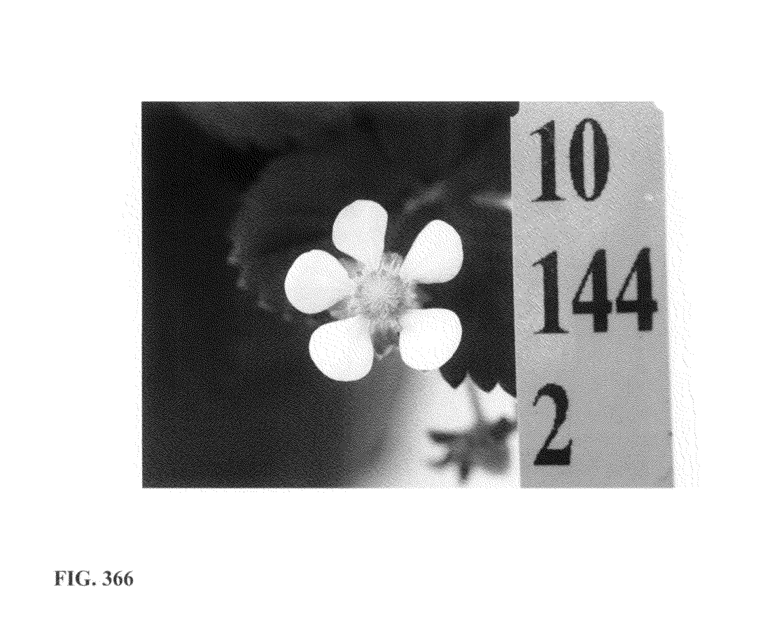

Cultivar ‘10.144-2’ has distinct disease/pathogen resistance, yield, appearance, size, and firmness. The plant of cultivar ‘10.144-2’ is characterized and distinguished in FIG. 364, the fruit of cultivar ‘10.144-2’ is characterized and distinguished in FIG. 365, and the flower of cultivar ‘10.144-2’ is characterized and distinguished in FIG. 366.

The following detailed description of cultivar ‘10.144-2’ is based on observations taken of plants and fruits (see FIGS. 364 and 365).

Certain characteristics of cultivar ‘10.144-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 122 provides data that characterizes the disease/pathogen resistance, yield, appearance, size, and firmness of cultivar ‘10.144-2’.

TABLE 122

| Cultivar | Vert | Fus | Yield | App | Size | Firm |
|---|---|---|---|---|---|---|
| 10.144-2 | 4.0 | 4.90 | 2113 | 3.7 | 36.6 | 10.5 |

### Cultivar ‘10.144-3’

In certain aspects, the present invention relates to a new and distinctive day-neutral type strawberry cultivar designated as ‘10.144-3’. Cultivar ‘10.144-3’ is typical of day-neutral strawberry cultivars and produces fruit regardless of day length when treated appropriately in arid, subtropical climates.

The strawberry cultivar ‘10.144-3’ originated from a cross between two unreleased germplasm accessions ‘07.120-3’ and ‘07.43-1’. The cultivar has been asexually reproduced by runners. The properties of this cultivar were found to be transmissible by such asexual reproduction. The cultivar is stable and reproduces true to type in successive generations of asexual reproduction.

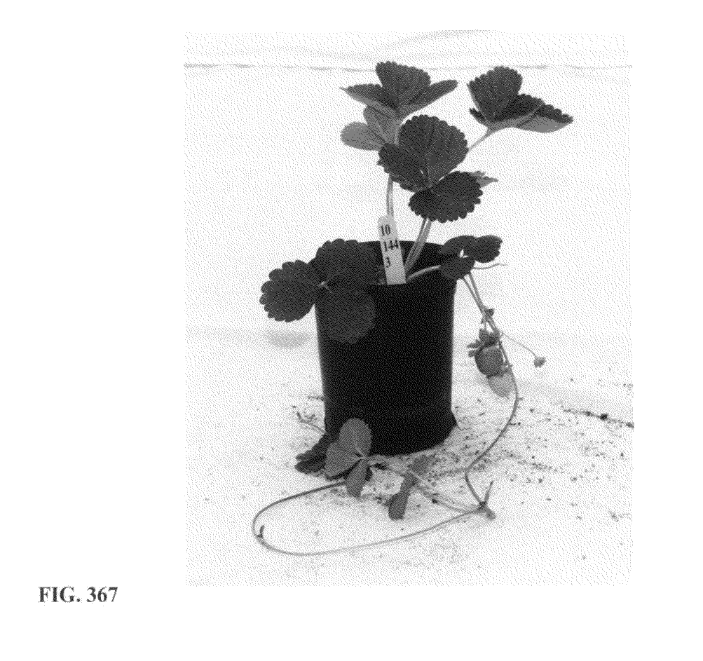

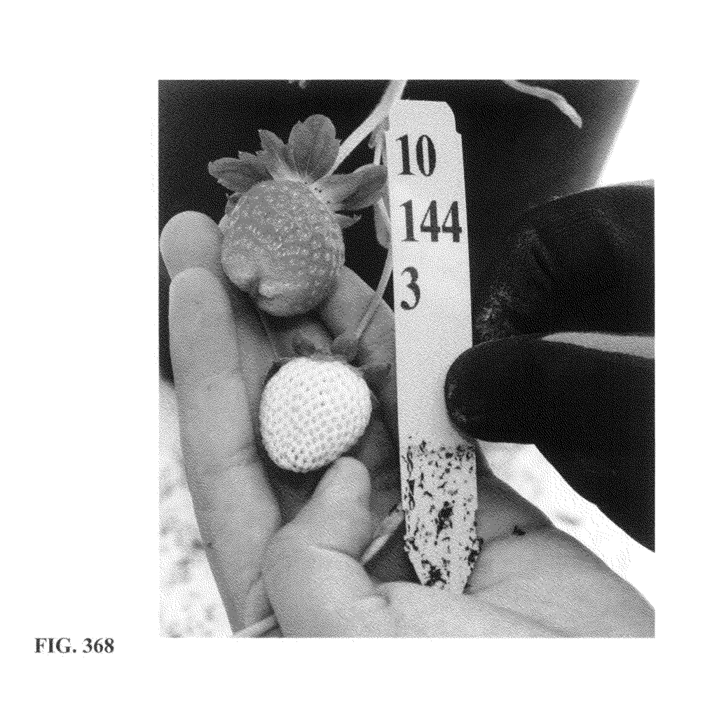

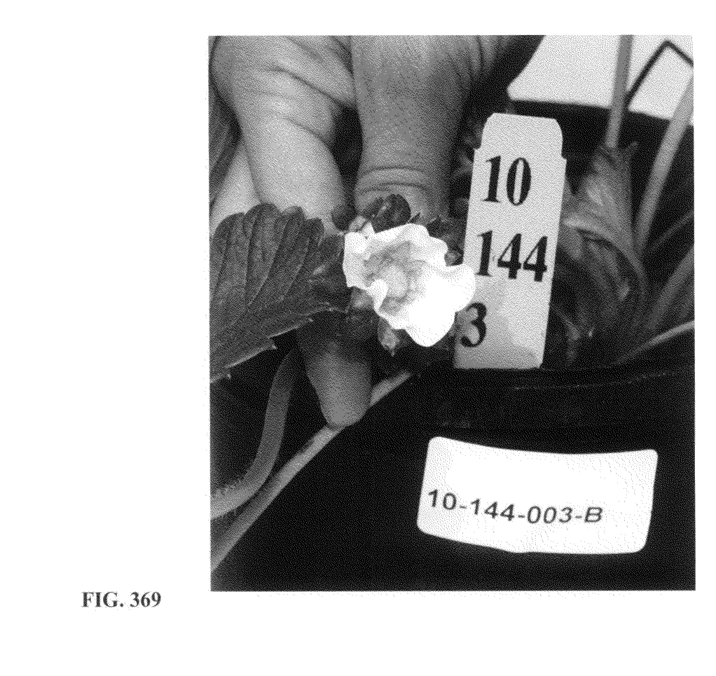

Cultivar ‘10.144-3’ has distinct disease/pathogen resistance, yield, appearance, size, and firmness. The plant of cultivar ‘10.144-3’ is characterized and distinguished in FIG. 367, the fruit of cultivar ‘10.144-3’ is characterized and distinguished in FIG. 368, and the flower of cultivar ‘10.144-3’ is characterized and distinguished in FIG. 369.

The following detailed description of cultivar ‘10.144-3’ is based on observations taken of plants and fruits (see FIGS. 367 and 368).

Certain characteristics of cultivar ‘10.144-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 123 provides data that characterizes the yield, appearance, size, and firmness of cultivar ‘10.144-3’.

TABLE 123

| Cultivar | Yield | App | Size | Firm |
|---|---|---|---|---|
| 10.144-3 | 2421 | 3.8 | 31.2 | 11.8 |

### Cultivar ‘10.160-1’

In certain aspects, the present invention relates to a new and distinctive day-neutral type strawberry cultivar designated as ‘10.160-1’. Cultivar ‘10.160-1’ is typical of day-neutral strawberry cultivars and produces fruit regardless of day length when treated appropriately in arid, subtropical climates.

The strawberry cultivar ‘10.160-1’ originated from a cross between two unreleased germplasm accessions ‘07.92-3’ and ‘06.111-1’. The cultivar has been asexually reproduced by runners. The properties of this cultivar were found to be transmissible by such asexual reproduction. The cultivar is stable and reproduces true to type in successive generations of asexual reproduction.

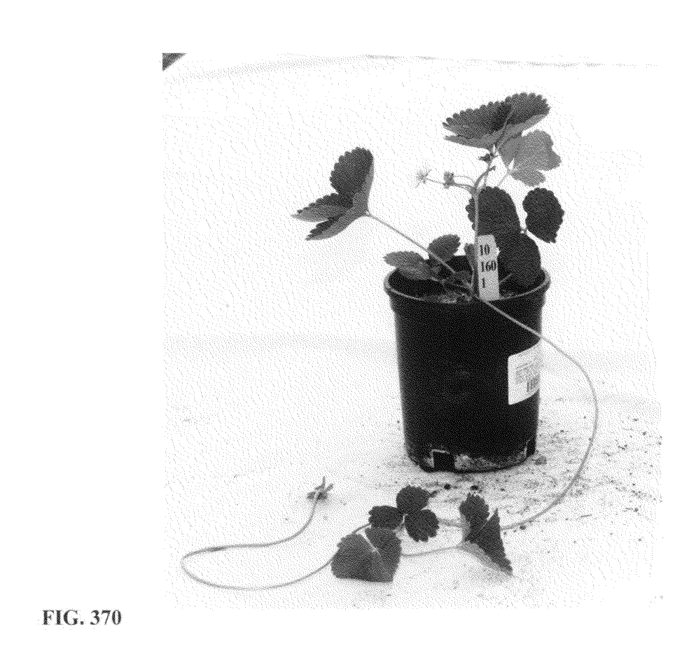

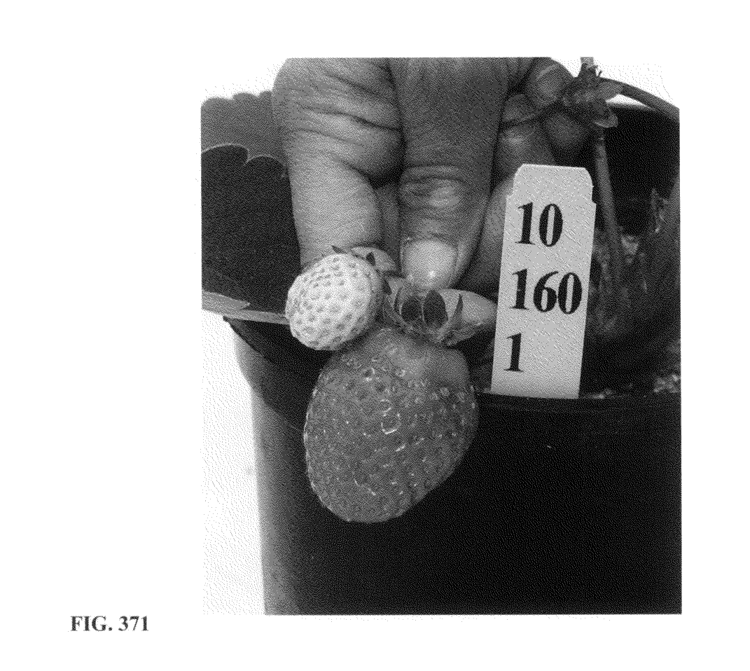

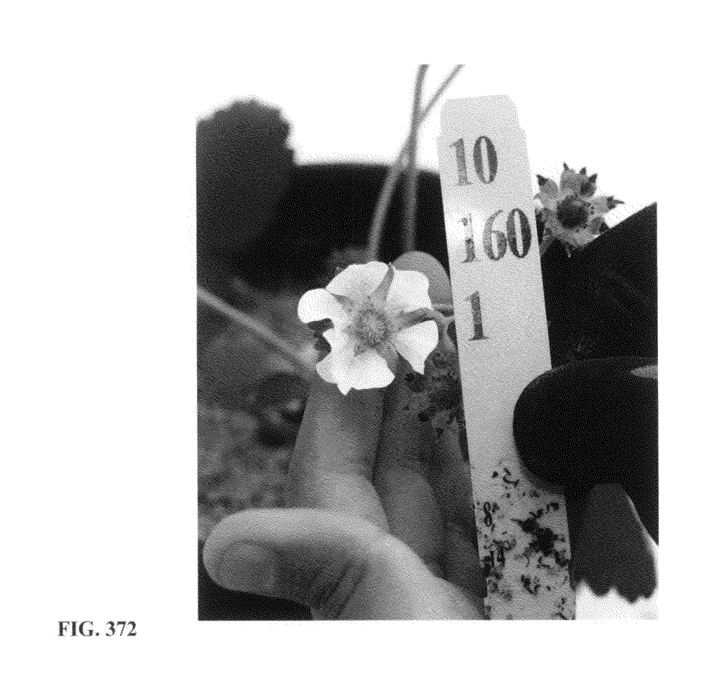

Cultivar ‘10.160-1’ has distinct disease/pathogen resistance, yield, appearance, size, and firmness. The plant of cultivar ‘10.160-1’ is characterized and distinguished in FIG. 370, the fruit of cultivar ‘10.160-1’ is characterized and distinguished in FIG. 371, and the flower of cultivar ‘10.160-1’ is characterized and distinguished in FIG. 372.

The following detailed description of cultivar ‘10.160-1’ is based on observations taken of plants and fruits (see FIGS. 370 and 371).

Certain characteristics of cultivar ‘10.160-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 124 provides data that characterizes the yield, appearance, size, and firmness of cultivar ‘10.160-1’.

TABLE 124

| Cultivar | Yield | App | Size | Firm |
|---|---|---|---|---|
| 10.160-1 | 1957 | 4.2 | 38.7 | 11.5 |

### Cultivar ‘10.160-9’

In certain aspects, the present invention relates to a new and distinctive day-neutral type strawberry cultivar designated as ‘10.160-9’. Cultivar ‘10.160-9’ is typical of day-neutral strawberry cultivars and produces fruit regardless of day length when treated appropriately in arid, subtropical climates.

The strawberry cultivar ‘10.160-9’ originated from a cross between two unreleased germplasm accessions ‘07.92-3’ and ‘06.111-1’. The cultivar has been asexually reproduced by runners. The properties of this cultivar were found to be transmissible by such asexual reproduction. The cultivar is stable and reproduces true to type in successive generations of asexual reproduction.

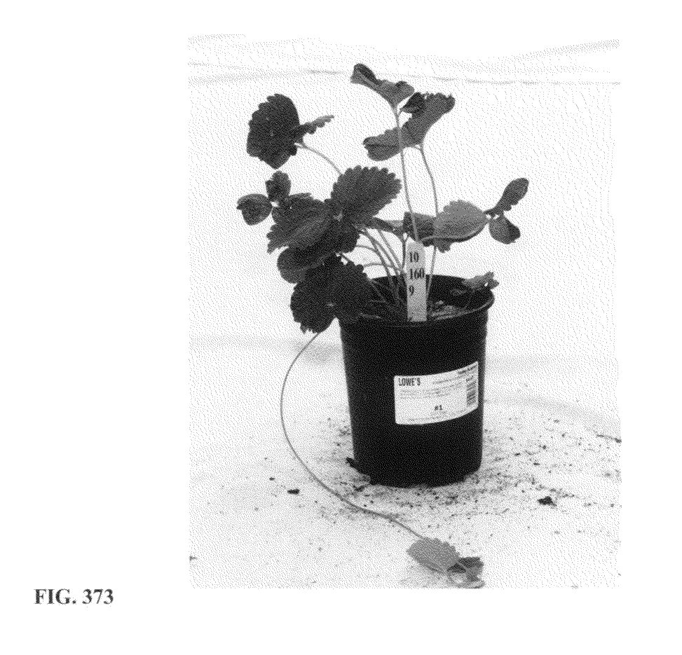

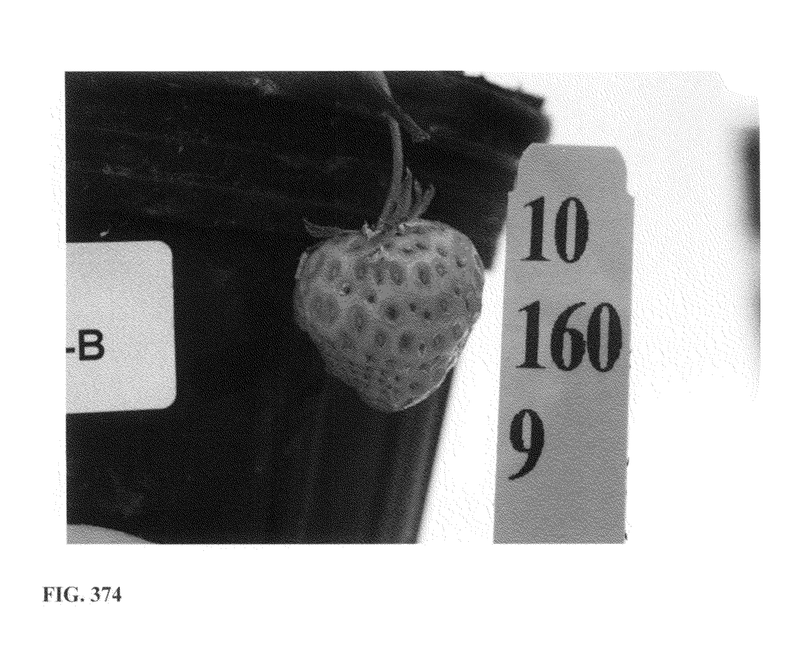

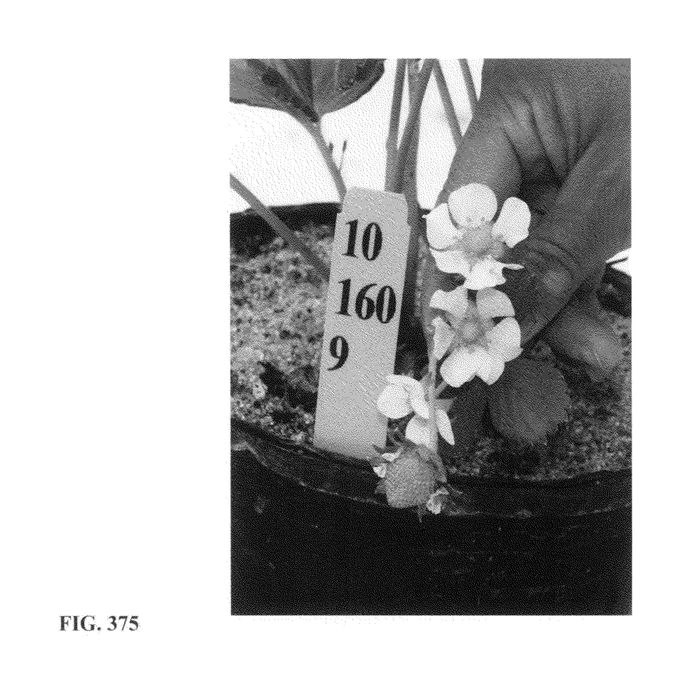

Cultivar ‘10.160-9’ has distinct disease/pathogen resistance, yield, appearance, size, and firmness. The plant of cultivar ‘10.160-9’ is characterized and distinguished in FIG. 373, the fruit of cultivar ‘10.160-9’ is characterized and distinguished in FIG. 374, and the flower of cultivar ‘10.160-9’ is characterized and distinguished in FIG. 375.

The following detailed description of cultivar ‘10.160-9’ is based on observations taken of plants and fruits (see FIGS. 373 and 374).

Certain characteristics of cultivar ‘10.160-9’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 125 provides data that characterizes the yield, appearance, size, and firmness of cultivar ‘10.160-9’.

TABLE 125

| Cultivar | Yield | App | Size | Firm |
|---|---|---|---|---|
| 10.160-9 | 2154 | 4.2 | 35.6 | 9.9 |

### Cultivar ‘10.169-1’

In certain aspects, the present invention relates to a new and distinctive short-day type strawberry cultivar designated as ‘10.169-1’. Cultivar ‘10.169-1’ is typical of short-day strawberry cultivars and produces fruit over an extended period when treated appropriately in arid, subtropical climates.

The strawberry cultivar ‘10.169-1’ originated from a cross between two unreleased germplasm accessions ‘05.106-2’ and ‘06.202-1’. The cultivar has been asexually reproduced by runners. The properties of this cultivar were found to be transmissible by such asexual reproduction. The cultivar is stable and reproduces true to type in successive generations of asexual reproduction.

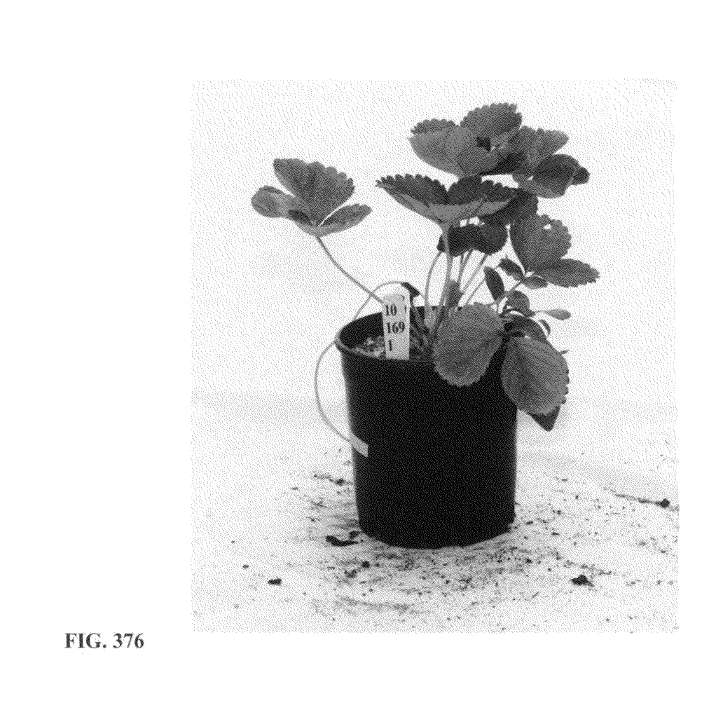

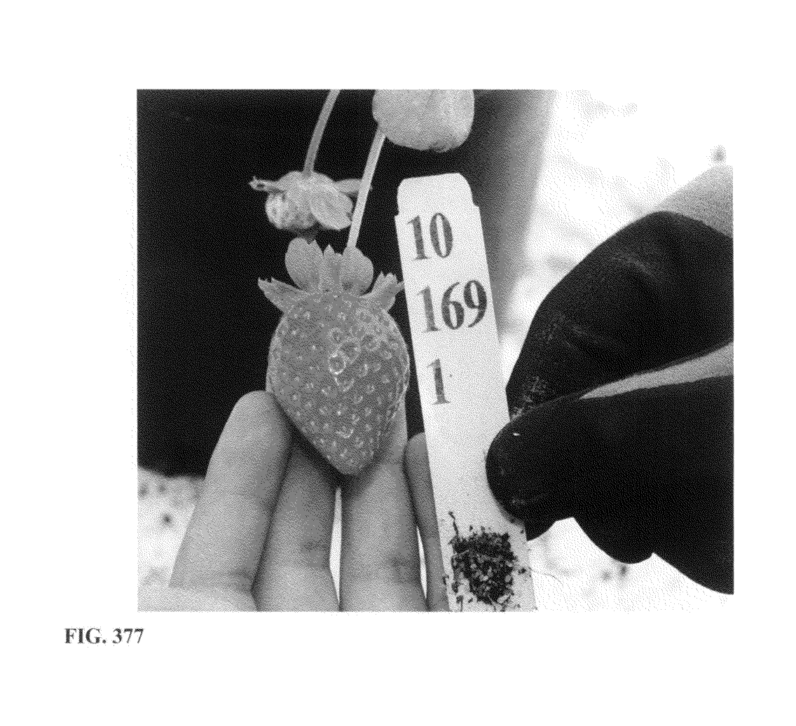

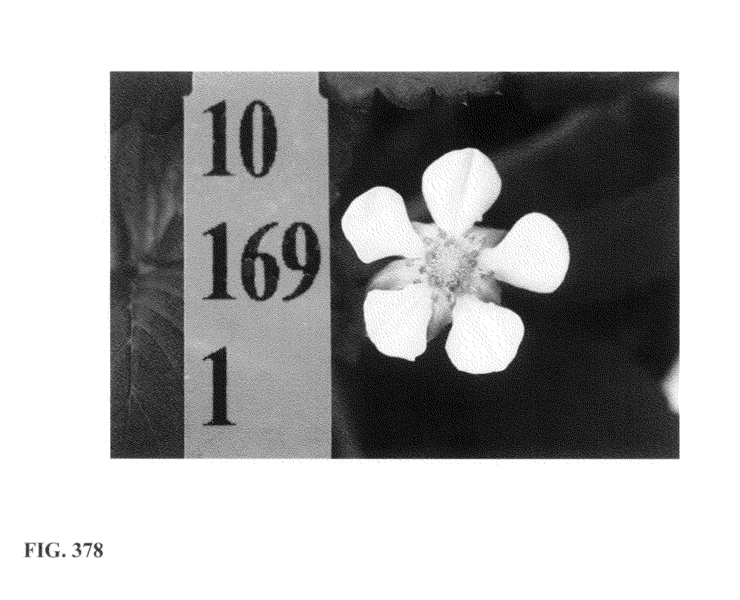

Cultivar ‘10.169-1’ has distinct disease/pathogen resistance, yield, appearance, size, and firmness. The plant of cultivar ‘10.169-1’ is characterized and distinguished in FIG. 376, the fruit of cultivar ‘10.169-1’ is characterized and distinguished in FIG. 377, and the flower of cultivar ‘10.169-1’ is characterized and distinguished in FIG. 378.

The following detailed description of cultivar ‘10.169-1’ is based on observations taken of plants and fruits (see FIGS. 376 and 377).

Certain characteristics of cultivar ‘10.169-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 126 provides data that characterizes the disease/pathogen resistance, yield, appearance, size, and firmness of cultivar ‘10.169-1’.

TABLE 126

| Cultivar | Vert | Fus | Yield | App | Size | Firm |
|---|---|---|---|---|---|---|
| 10.169-1 | 3.7 | 2.9 | 1814 | 4.1 | 35.8 | 10.4 |

### Cultivar ‘10.187-4’

In certain aspects, the present invention relates to a new and distinctive day-neutral type strawberry cultivar designated as ‘10.187-4’. Cultivar ‘10.187-4’ is typical of day-neutral strawberry cultivars and produces fruit regardless of day length when treated appropriately in arid, subtropical climates.

The strawberry cultivar ‘10.187-4’ originated from a cross between two unreleased germplasm accessions ‘07.92-3’ and ‘07.43-1’. The cultivar has been asexually reproduced by runners. The properties of this cultivar were found to be transmissible by such asexual reproduction. The cultivar is stable and reproduces true to type in successive generations of asexual reproduction.

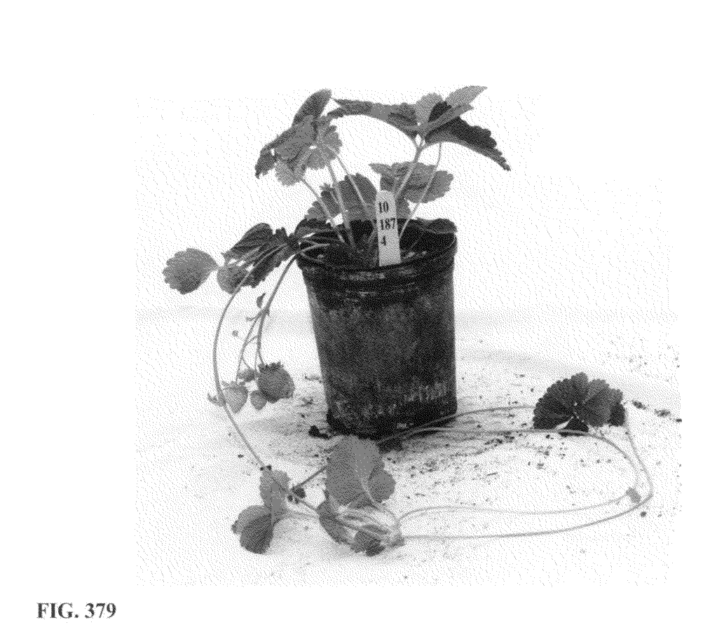

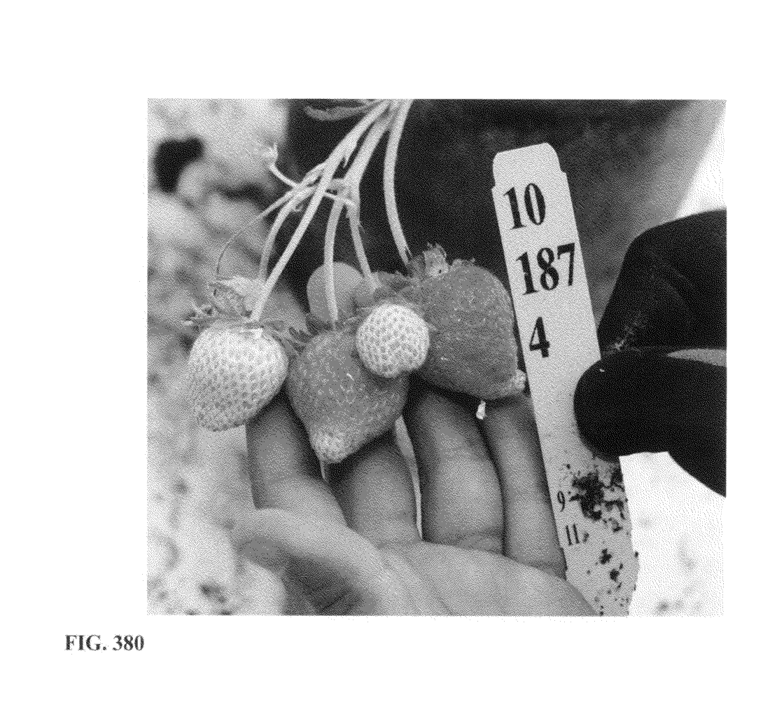

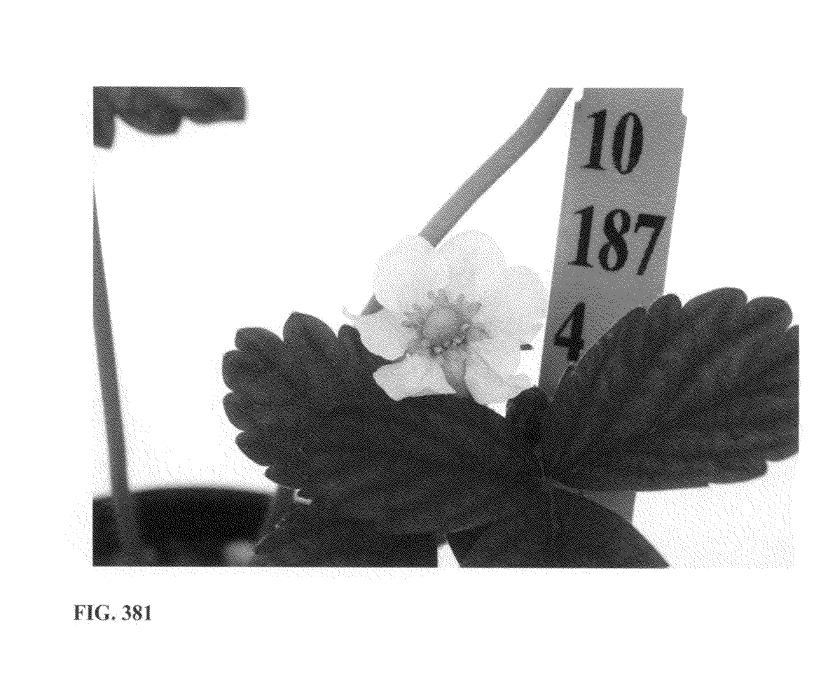

Cultivar ‘10.187-4’ has distinct disease/pathogen resistance, yield, appearance, size, and firmness. The plant of cultivar ‘10.187-4’ is characterized and distinguished in FIG. 379, the fruit of cultivar ‘10.187-4’ is characterized and distinguished in FIG. 380, and the flower of cultivar ‘10.187-4’ is characterized and distinguished in FIG. 381.

The following detailed description of cultivar ‘10.187-4’ is based on observations taken of plants and fruits (see FIGS. 379 and 380).

Certain characteristics of cultivar ‘10.187-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 127 provides data that characterizes the disease/pathogen resistance, yield, appearance, size, and firmness of cultivar ‘10.187-4’.

TABLE 127

| Cultivar | Vert | Yield | App | Size | Firm |
|---|---|---|---|---|---|
| 10.187-4 | 4.2 | 2200 | 3.9 | 34.0 | 12.8 |

### Cultivar ‘11.21-1’

In certain aspects, the present invention relates to a new and distinctive short-day type strawberry cultivar designated as ‘11.21-1’. Cultivar ‘11.21-1’ is typical of short-day strawberry cultivars and produces fruit over an extended period when treated appropriately in arid, subtropical climates.

The strawberry cultivar ‘11.21-1’ originated from a cross between two unreleased germplasm accessions ‘07.120-3’ and ‘08.75-1’. The cultivar has been asexually reproduced by runners. The properties of this cultivar were found to be transmissible by such asexual reproduction. The cultivar is stable and reproduces true to type in successive generations of asexual reproduction.

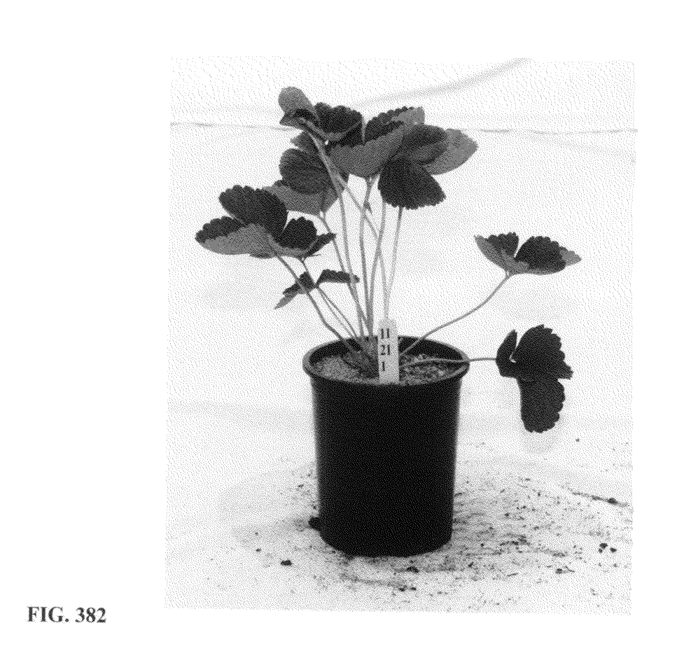

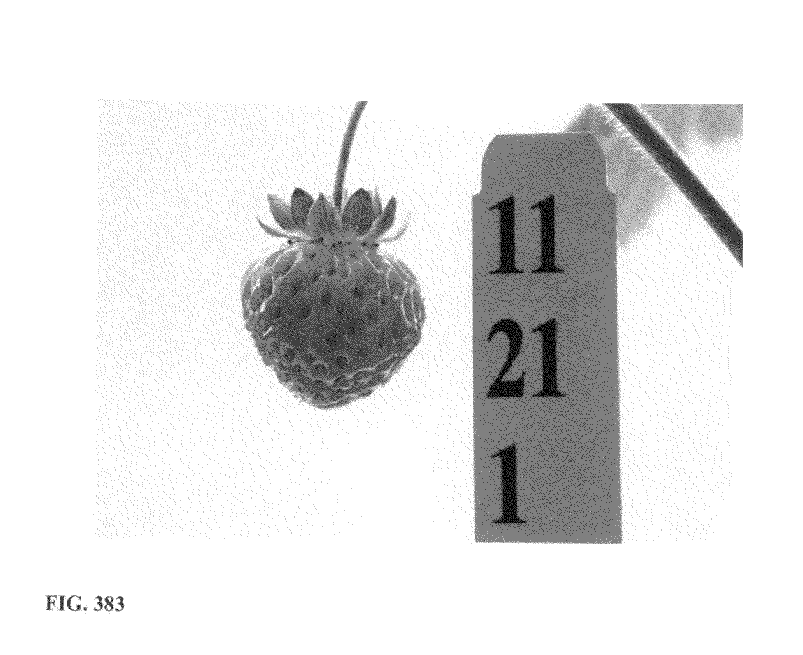

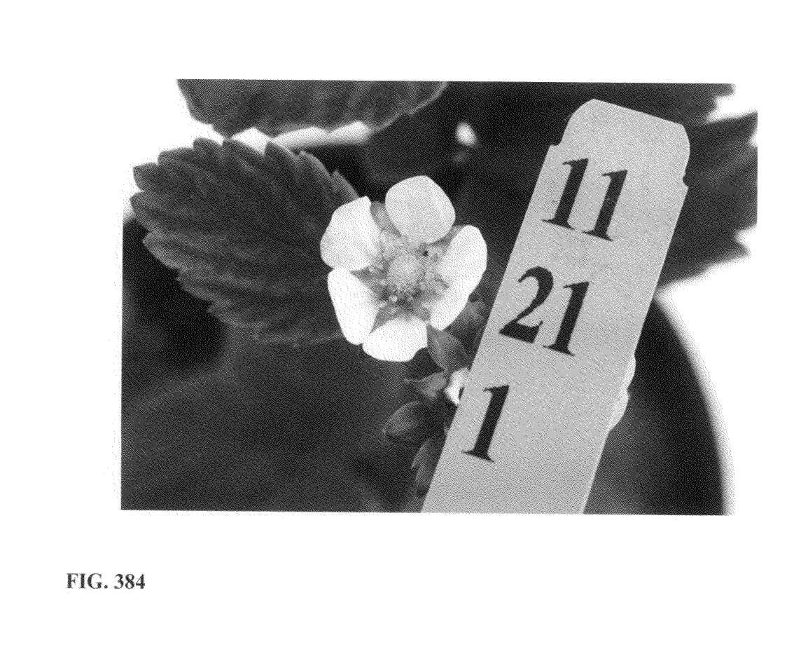

Cultivar ‘11.21-1’ has distinct disease/pathogen resistance, yield, appearance, size, and firmness. The plant of cultivar ‘11.21-1’ is characterized and distinguished in FIG. 382, the fruit of cultivar ‘11.21-1’ is characterized and distinguished in FIG. 383, and the flower of cultivar ‘11.21-1’ is characterized and distinguished in FIG. 384.

The following detailed description of cultivar ‘11.21-1’ is based on observations taken of plants and fruits (see FIGS. 382 and 383).

Certain characteristics of cultivar ‘11.21-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 128 provides data that characterizes the yield, appearance, size, and firmness of cultivar ‘11.21-1’.

TABLE 128

| Cultivar | Yield | App | Size | Firm |
|---|---|---|---|---|
| 11.21-1 | 2661 | 4.0 | 32.0 | 12.5 |

### Cultivar ‘11.32-2’

In certain aspects, the present invention relates to a new and distinctive short-day type strawberry cultivar designated as ‘11.32-2’. Cultivar ‘11.32-2’ is typical of short-day strawberry cultivars and produces fruit over an extended period when treated appropriately in arid, subtropical climates.

The strawberry cultivar ‘11.32-2’ originated from a cross between two unreleased germplasm accessions ‘07.148-1’ and ‘07.67-608’. The cultivar has been asexually reproduced by runners. The properties of this cultivar were found to be transmissible by such asexual reproduction. The cultivar is stable and reproduces true to type in successive generations of asexual reproduction.

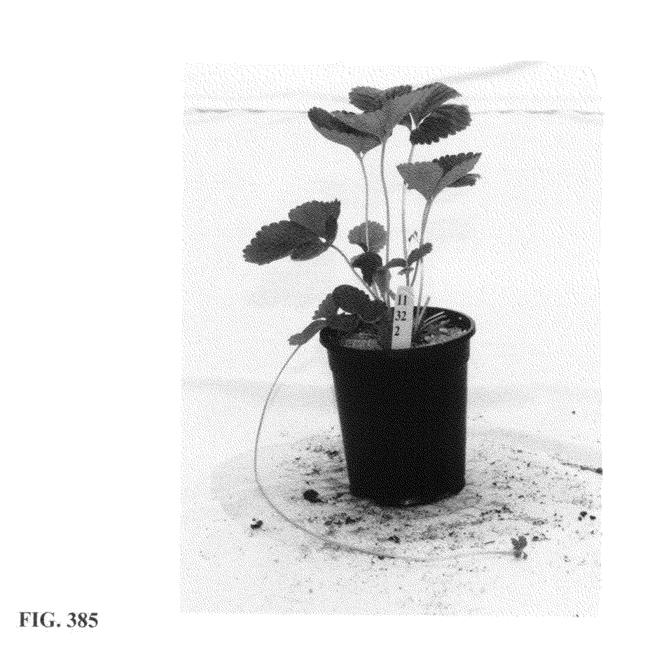

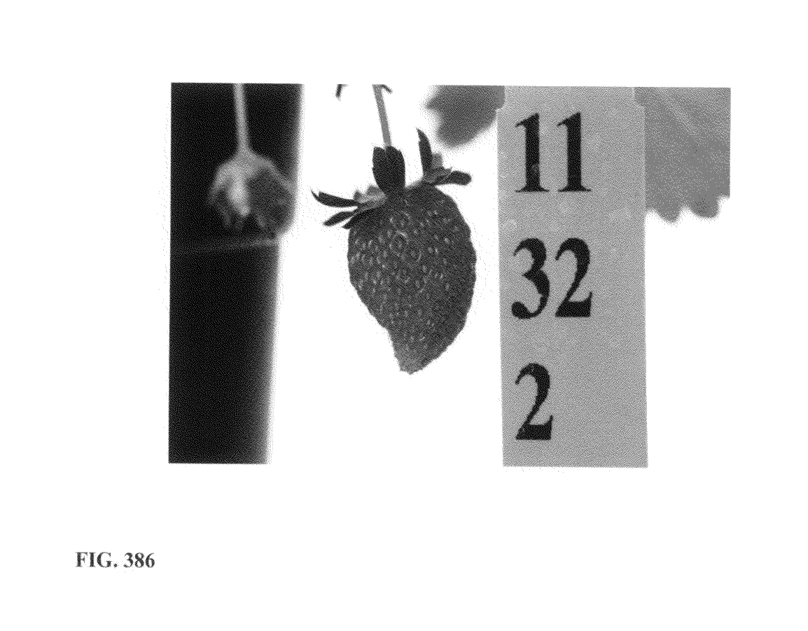

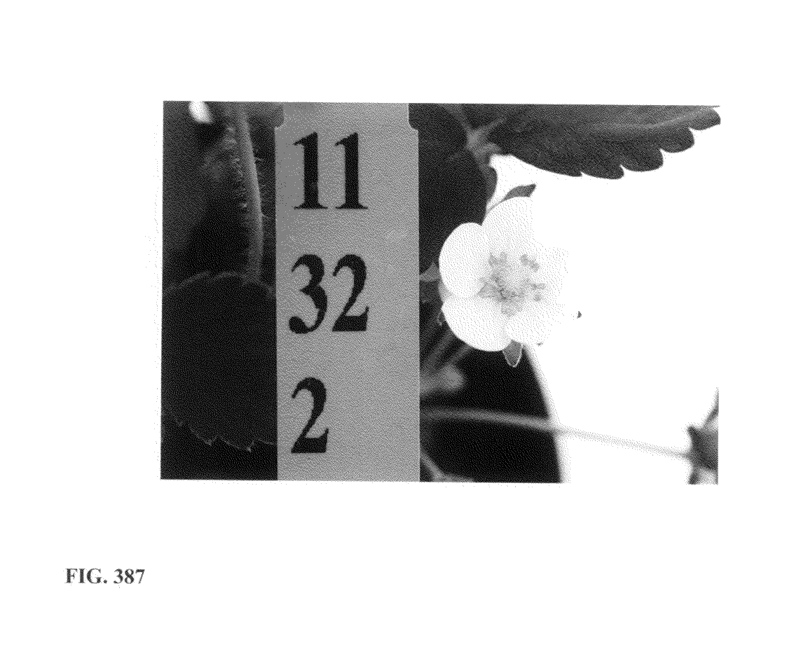

Cultivar ‘11.32-2’ has distinct disease/pathogen resistance, yield, appearance, size, and firmness. The plant of cultivar ‘11.32-2’ is characterized and distinguished in FIG. 385, the fruit of cultivar ‘11.32-2’ is characterized and distinguished in FIG. 386, and the flower of cultivar ‘11.32-2’ is characterized and distinguished in FIG. 387.

The following detailed description of cultivar ‘11.32-2’ is based on observations taken of plants and fruits (see FIGS. 385 and 386).

Certain characteristics of cultivar ‘11.32-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 129 provides data that characterizes the yield, appearance, size, and firmness of cultivar ‘11.32-2’.

TABLE 129

| Cultivar | Yield | App | Size | Firm |
|---|---|---|---|---|
| 11.32-2 | 2861 | 3.6 | 34.7 | 10.4 |

### Cultivar ‘11.34-605’

In certain aspects, the present invention relates to a new and distinctive short-day type strawberry cultivar designated as ‘11.34-605’. Cultivar ‘11.34-605’ is typical of short-day strawberry cultivars and produces fruit over an extended period when treated appropriately in arid, subtropical climates.

The strawberry cultivar ‘11.34-605’ originated from a cross between unreleased germplasm accession ‘06.51-609’ and cultivar ‘Grenada’. The cultivar has been asexually reproduced by runners. The properties of this cultivar were found to be transmissible by such asexual reproduction. The cultivar is stable and reproduces true to type in successive generations of asexual reproduction.

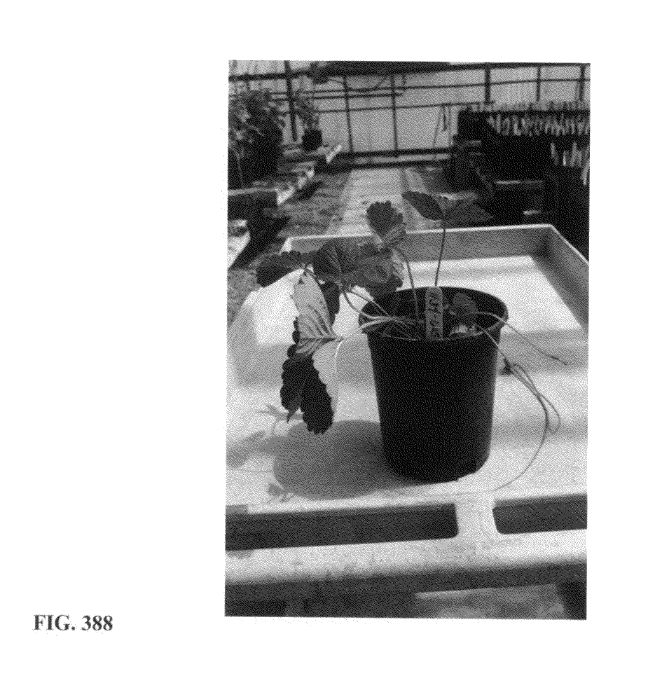

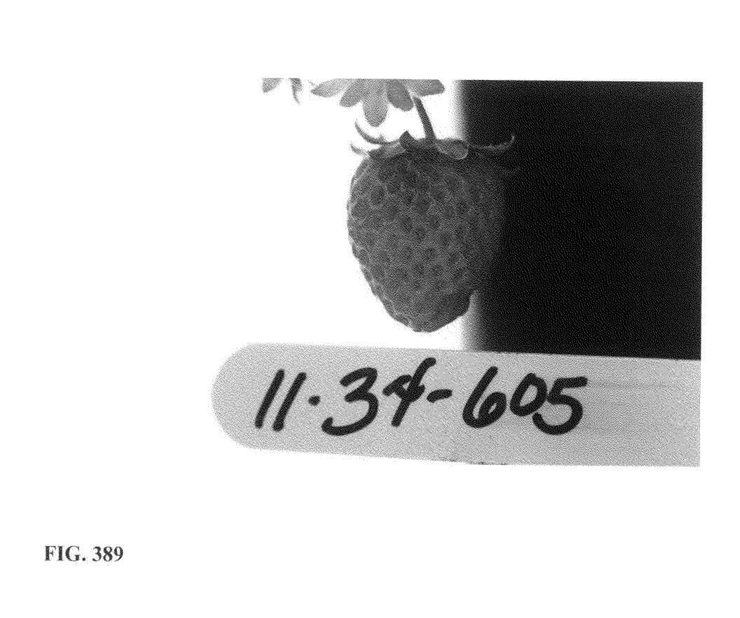

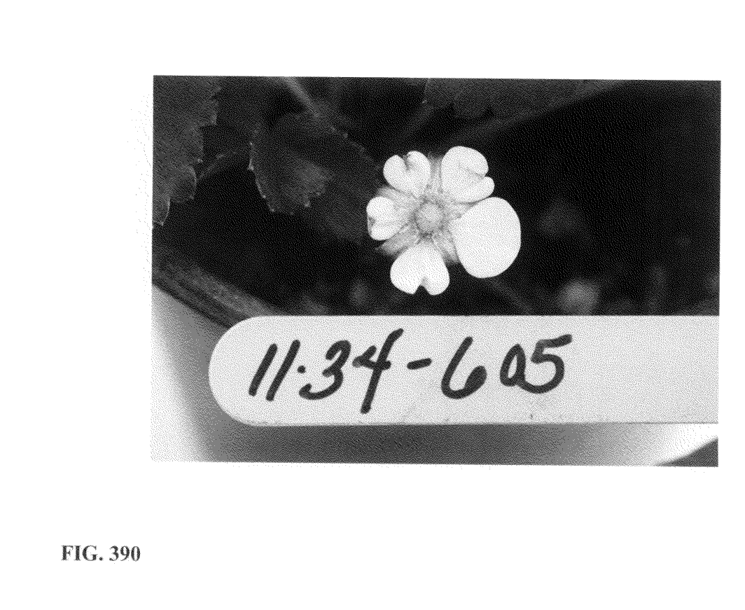

Cultivar ‘11.34-605’ has distinct disease/pathogen resistance, yield, appearance, size, and firmness. The plant of cultivar ‘11.34-605’ is characterized and distinguished in FIG. 388, the fruit of cultivar ‘11.34-605’ is characterized and distinguished in FIG. 389, and the flower of cultivar ‘11.34-605’ is characterized and distinguished in FIG. 390.

The following detailed description of cultivar ‘11.34-605’ is based on observations taken of plants and fruits (see FIGS. 388 and 389).

Certain characteristics of cultivar ‘11.34-605’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 130 provides data that characterizes the yield, appearance, size, and firmness of cultivar ‘11.34-605’.

TABLE 130

| Cultivar | Yield | App | Size | Firm |
|---|---|---|---|---|
| 11.34-605 | 1084 | 4.2 | 36.0 | 7.3 |

### Cultivar ‘11.36-601’

In certain aspects, the present invention relates to a new and distinctive short-day type strawberry cultivar designated as ‘11.36-601’. Cultivar ‘11.36-601’ is typical of short-day strawberry cultivars and produces fruit over an extended period when treated appropriately in arid, subtropical climates.

The strawberry cultivar ‘11.36-601’ originated from a cross between unreleased germplasm accession ‘07.64-5’ and cultivar ‘Grenada’. The cultivar has been asexually reproduced by runners. The properties of this cultivar were found to be transmissible by such asexual reproduction. The cultivar is stable and reproduces true to type in successive generations of asexual reproduction.

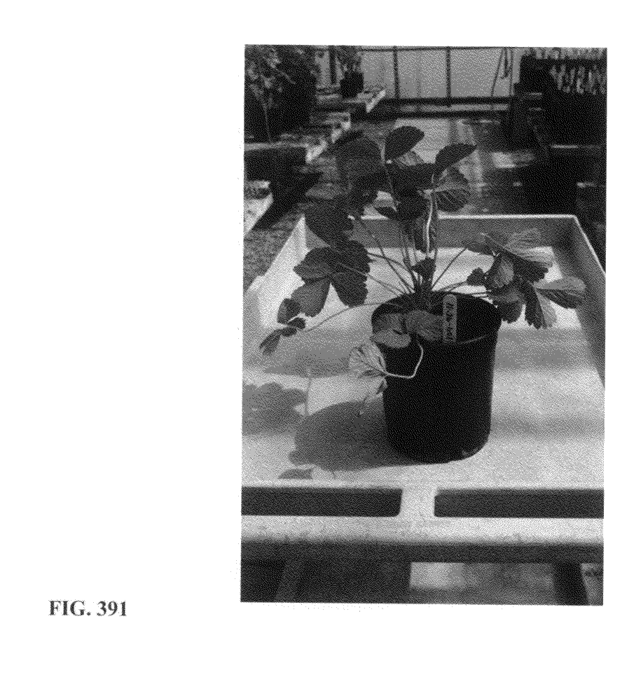

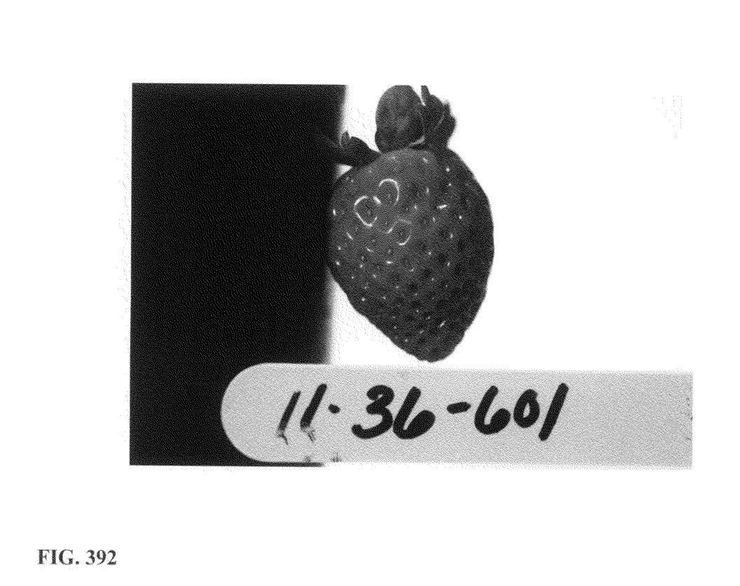

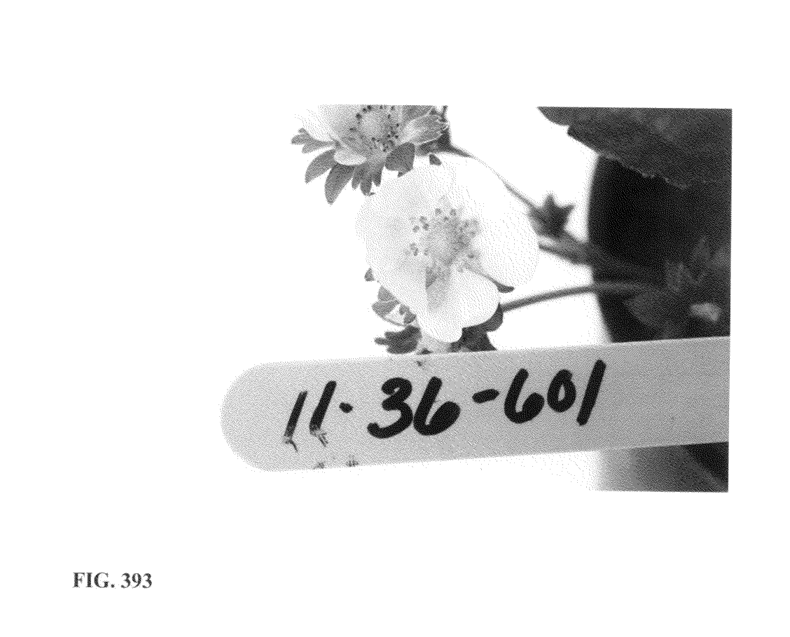

Cultivar ‘11.36-601’ has distinct disease/pathogen resistance, yield, appearance, size, and firmness. The plant of cultivar ‘11.36-601’ is characterized and distinguished in FIG. 391, the fruit of cultivar ‘11.36-601’ is characterized and distinguished in FIG. 392, and the flower of cultivar ‘11.36-601’ is characterized and distinguished in FIG. 393.

The following detailed description of cultivar ‘11.36-601’ is based on observations taken of plants and fruits (see FIGS. 391 and 392).

Certain characteristics of cultivar ‘11.36-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 131 provides data that characterizes the yield, appearance, size, and firmness of cultivar ‘11.36-601’.

TABLE 131

| Cultivar | Yield | App | Size | Firm |
|---|---|---|---|---|
| 11.36-601 | 1192 | 3.9 | 41.0 | 8.5 |

### Cultivar ‘11.39-1’

In certain aspects, the present invention relates to a new and distinctive short-day type strawberry cultivar designated as ‘11.39-1’. Cultivar ‘11.39-1’ is typical of short-day strawberry cultivars and produces fruit over an extended period when treated appropriately in arid, subtropical climates.

The strawberry cultivar ‘11.39-1’ originated from a cross between unreleased germplasm accession ‘08.182-2’ and cultivar ‘Grenada’. The cultivar has been asexually reproduced by runners. The properties of this cultivar were found to be transmissible by such asexual reproduction. The cultivar is stable and reproduces true to type in successive generations of asexual reproduction.

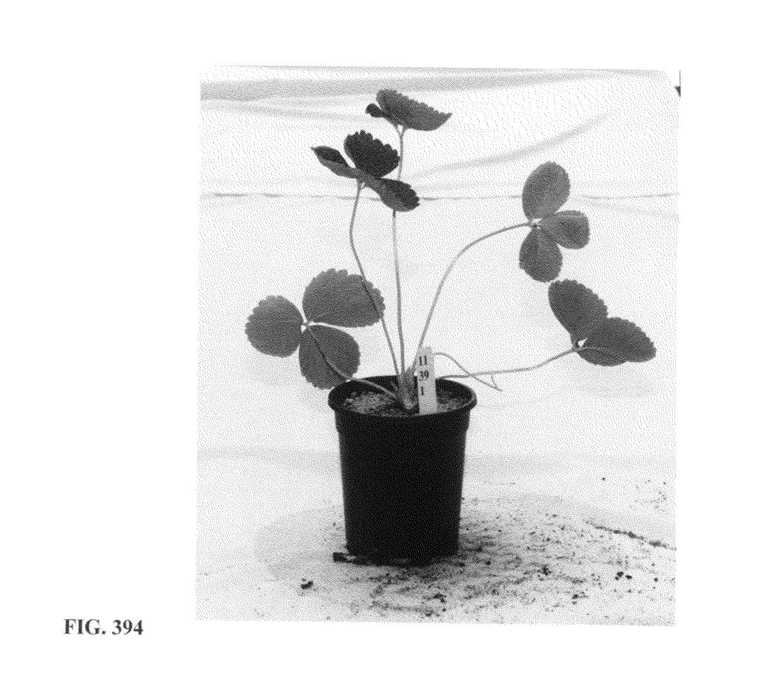

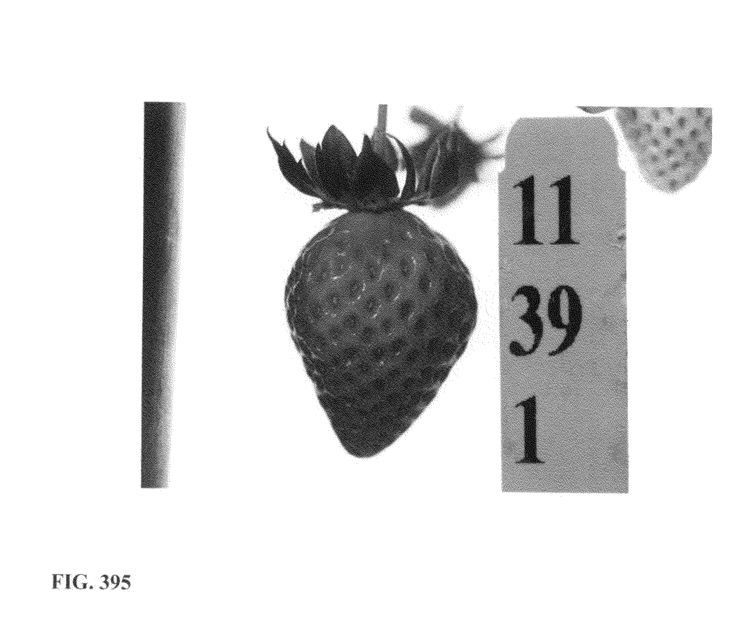

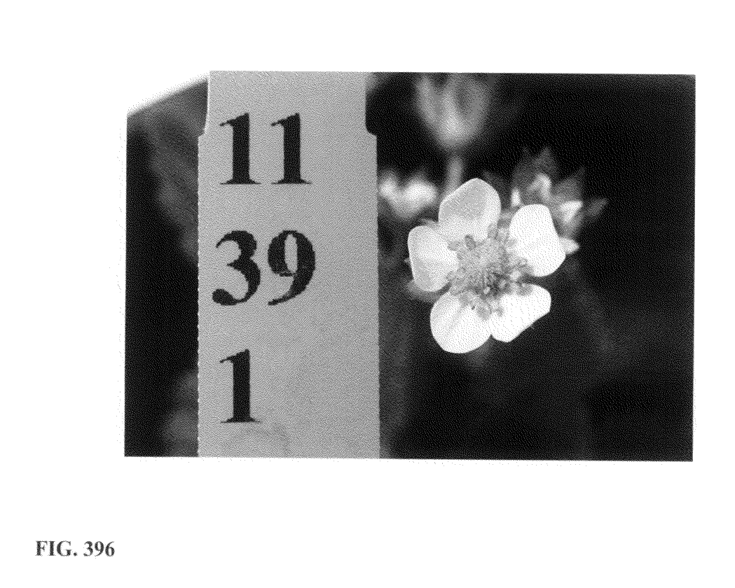

Cultivar ‘11.39-1’ has distinct disease/pathogen resistance, yield, appearance, size, and firmness. The plant of cultivar ‘11.39-1’ is characterized and distinguished in FIG. 394, the fruit of cultivar ‘11.39-1’ is characterized and distinguished in FIG. 395, and the flower of cultivar ‘11.39-1’ is characterized and distinguished in FIG. 396.

The following detailed description of cultivar ‘11.39-1’ is based on observations taken of plants and fruits (see FIGS. 394 and 395).

Certain characteristics of cultivar ‘11.39-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 132 provides data that characterizes the yield, appearance, size, and firmness of cultivar ‘11.39-1’.

TABLE 132

| Cultivar | Yield | App | Size | Firm |
|---|---|---|---|---|
| 11.39-1 | 2397 | 3.7 | 36.0 | 12.3 |

### Cultivar ‘11.49-6’

In certain aspects, the present invention relates to a new and distinctive short-day type strawberry cultivar designated as ‘11.49-6’. Cultivar ‘11.49-6’ is typical of short-day strawberry cultivars and produces fruit over an extended period when treated appropriately in arid, subtropical climates.

The strawberry cultivar ‘11.49-6’ originated from a cross between two unreleased germplasm accessions ‘07.120-3’ and ‘08.132-3’. The cultivar has been asexually reproduced by runners. The properties of this cultivar were found to be transmissible by such asexual reproduction. The cultivar is stable and reproduces true to type in successive generations of asexual reproduction.

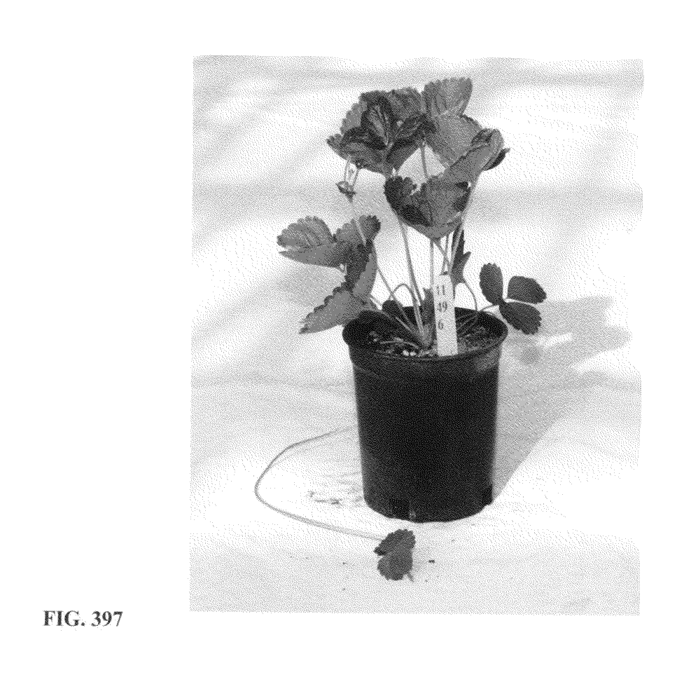

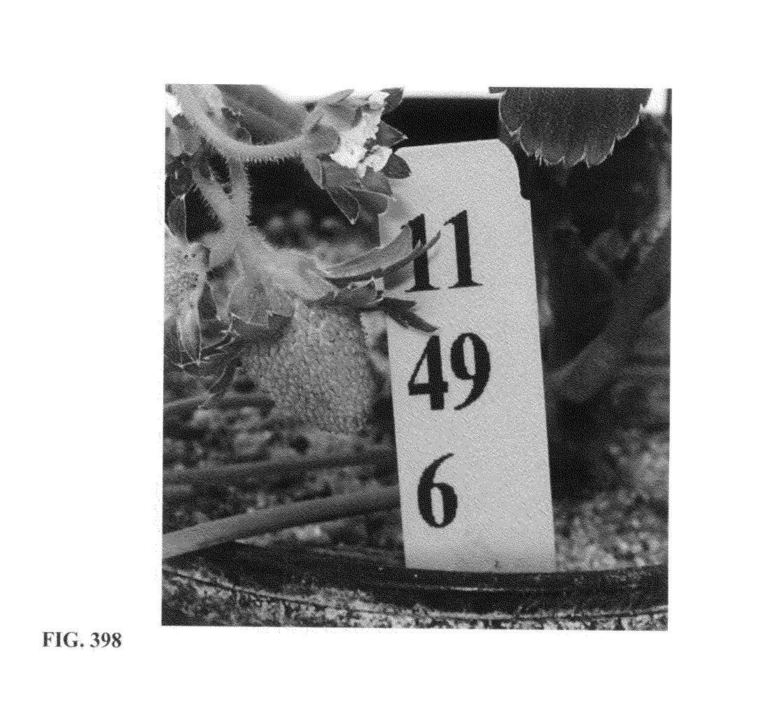

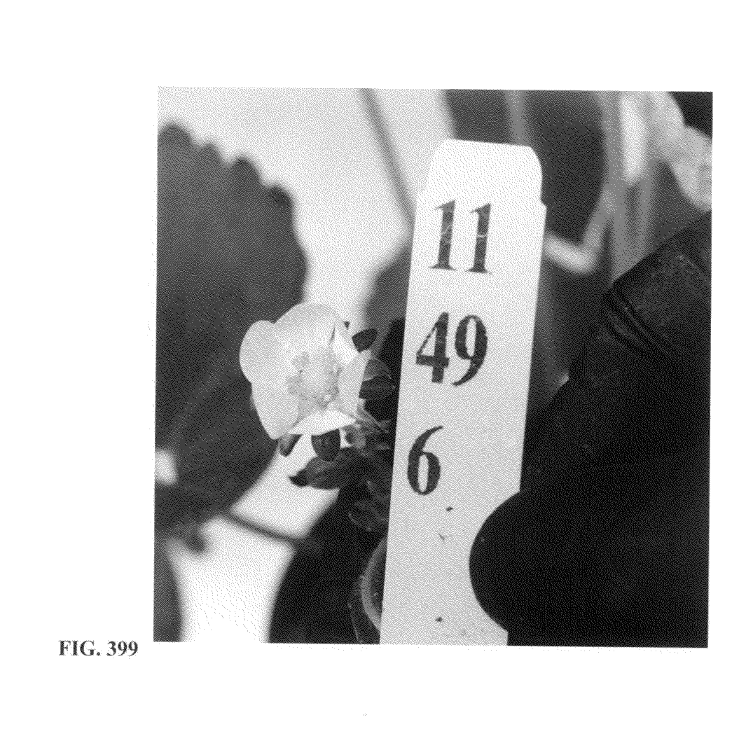

Cultivar ‘11.49-6’ has distinct disease/pathogen resistance, yield, appearance, size, and firmness. The plant of cultivar ‘11.49-6’ is characterized and distinguished in FIG. 397, the fruit of cultivar ‘11.49-6’ is characterized and distinguished in FIG. 398, and the flower of cultivar ‘11.49-6’ is characterized and distinguished in FIG. 399.

The following detailed description of cultivar ‘11.49-6’ is based on observations taken of plants and fruits (see FIGS. 397 and 398).

Certain characteristics of cultivar ‘11.49-6’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 133 provides data that characterizes the yield, appearance, size, and firmness of cultivar ‘11.49-6’.

TABLE 133

| Cultivar | Yield | App | Size | Firm |
|---|---|---|---|---|
| 11.49-6 | 3035 | 4.1 | 35.5 | 10.8 |

### Cultivar ‘11.50-1’

In certain aspects, the present invention relates to a new and distinctive short-day type strawberry cultivar designated as ‘11.50-1’. Cultivar ‘11.50-1’ is typical of short-day strawberry cultivars and produces fruit over an extended period when treated appropriately in arid, subtropical climates.

The strawberry cultivar ‘11.50-1’ originated from a cross between two unreleased germplasm accessions ‘07.148-1’ and ‘08.132-3’. The cultivar has been asexually reproduced by runners. The properties of this cultivar were found to be transmissible by such asexual reproduction. The cultivar is stable and reproduces true to type in successive generations of asexual reproduction.

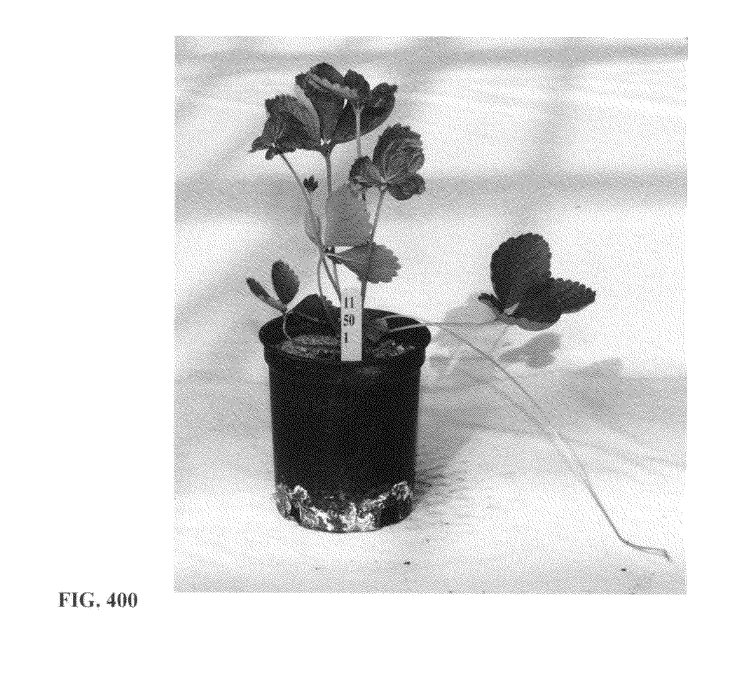

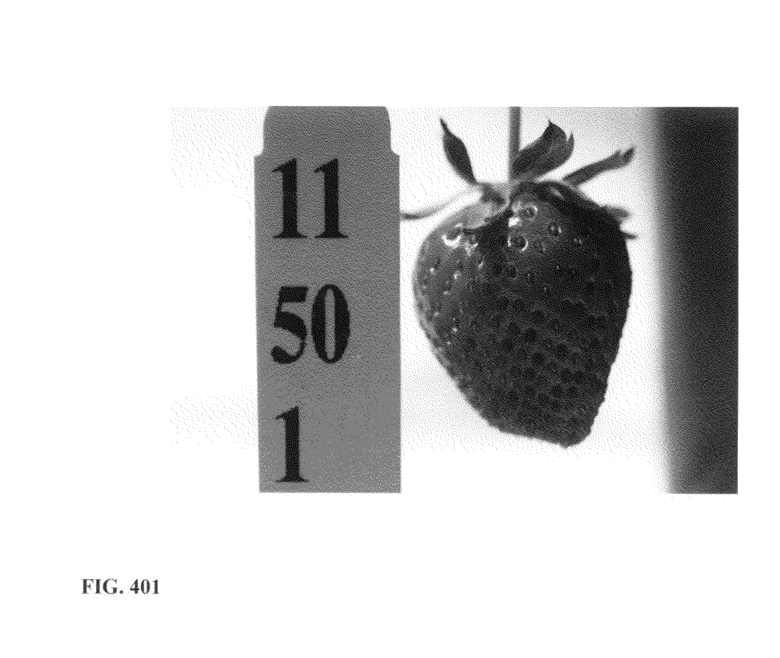

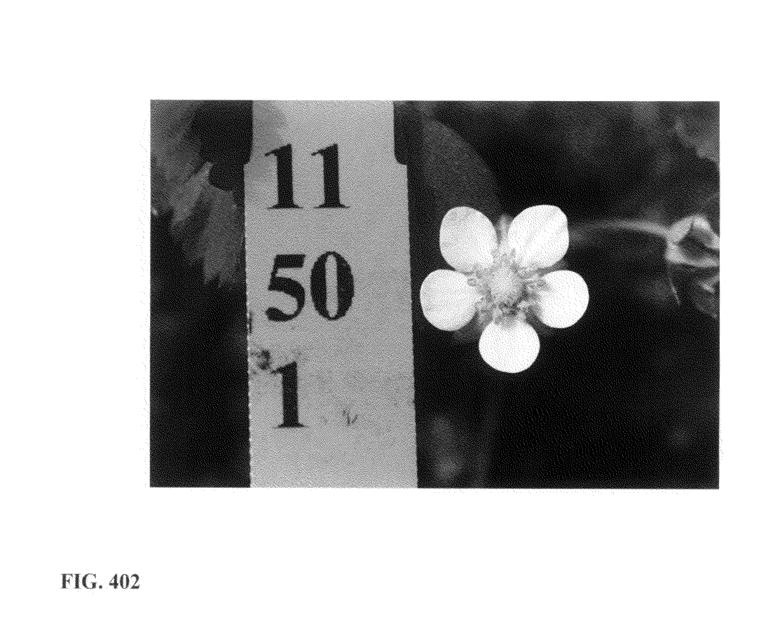

Cultivar ‘11.50-1’ has distinct disease/pathogen resistance, yield, appearance, size, and firmness. The plant of cultivar ‘11.50-1’ is characterized and distinguished in FIG. 400, the fruit of cultivar ‘11.50-1’ is characterized and distinguished in FIG. 401, and the flower of cultivar ‘11.50-1’ is characterized and distinguished in FIG. 402.

The following detailed description of cultivar ‘11.50-1’ is based on observations taken of plants and fruits (see FIGS. 400 and 401).

Certain characteristics of cultivar ‘11.50-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 134 provides data that characterizes the yield, appearance, size, and firmness of cultivar ‘11.50-1’.

TABLE 134

| Cultivar | Yield | App | Size | Firm |
|---|---|---|---|---|
| 11.50-1 | 2221 | 3.3 | 29.1 | 11.1 |

### Cultivar ‘11.51-1’

In certain aspects, the present invention relates to a new and distinctive short-day type strawberry cultivar designated as ‘11.51-1’. Cultivar ‘11.51-1’ is typical of short-day strawberry cultivars and produces fruit over an extended period when treated appropriately in arid, subtropical climates.

The strawberry cultivar ‘11.51-1’ originated from a cross between two unreleased germplasm accessions ‘08.182-2’ and ‘08.132-3’. The cultivar has been asexually reproduced by runners. The properties of this cultivar were found to be transmissible by such asexual reproduction. The cultivar is stable and reproduces true to type in successive generations of asexual reproduction.

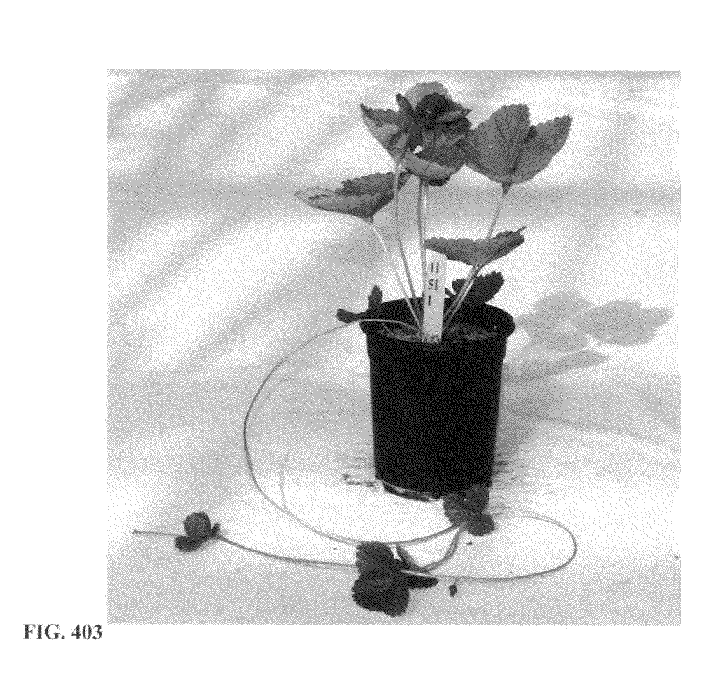

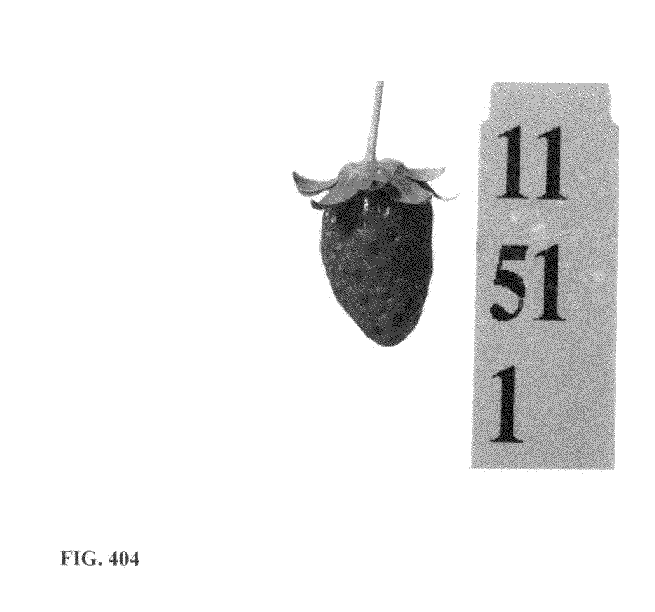

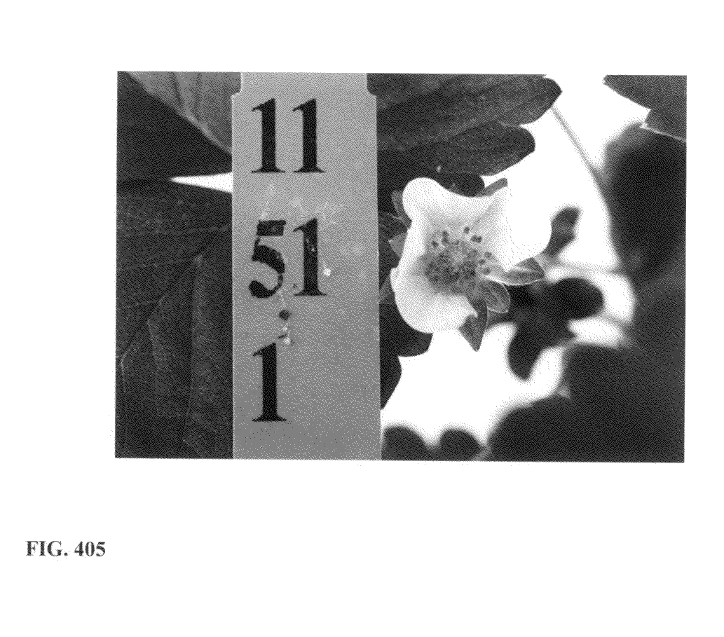

Cultivar ‘11.51-1’ has distinct disease/pathogen resistance, yield, appearance, size, and firmness. The plant of cultivar ‘11.51-1’ is characterized and distinguished in FIG. 403, the fruit of cultivar ‘11.51-1’ is characterized and distinguished in FIG. 404, and the flower of cultivar ‘11.51-1’ is characterized and distinguished in FIG. 405.

The following detailed description of cultivar ‘11.51-1’ is based on observations taken of plants and fruits (see FIGS. 403 and 404).

Certain characteristics of cultivar ‘11.51-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 135 provides data that characterizes the yield, appearance, size, and firmness of cultivar ‘11.51-1’.

TABLE 135

| Cultivar | Yield | App | Size | Firm |
|---|---|---|---|---|
| 11.51-1 | 1733 | 3.7 | 30.8 | 11.1 |

### Cultivar ‘11.55-4’

In certain aspects, the present invention relates to a new and distinctive short-day type strawberry cultivar designated as ‘11.55-4’. Cultivar ‘11.55-4’ is typical of short-day strawberry cultivars and produces fruit over an extended period when treated appropriately in arid, subtropical climates.

The strawberry cultivar ‘11.55-4’ originated from a cross between two unreleased germplasm accessions ‘07.120-3’ and ‘08.138-2’. The cultivar has been asexually reproduced by runners. The properties of this cultivar were found to be transmissible by such asexual reproduction. The cultivar is stable and reproduces true to type in successive generations of asexual reproduction.

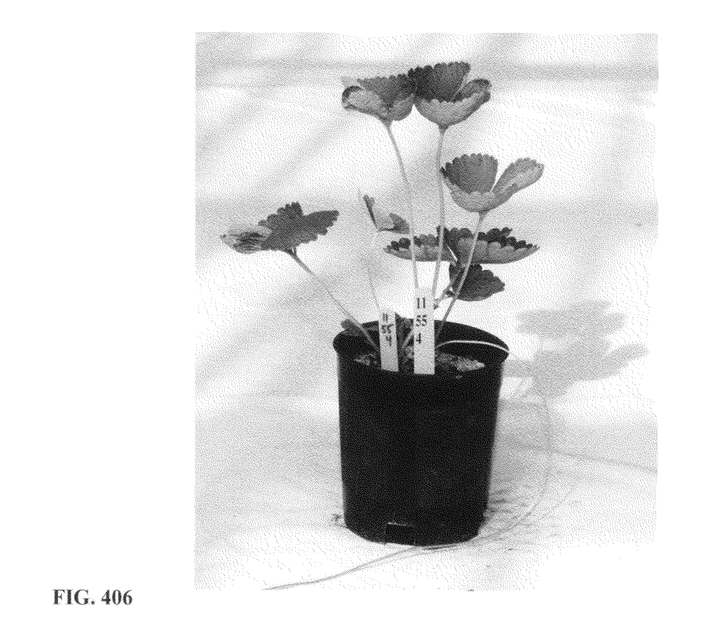

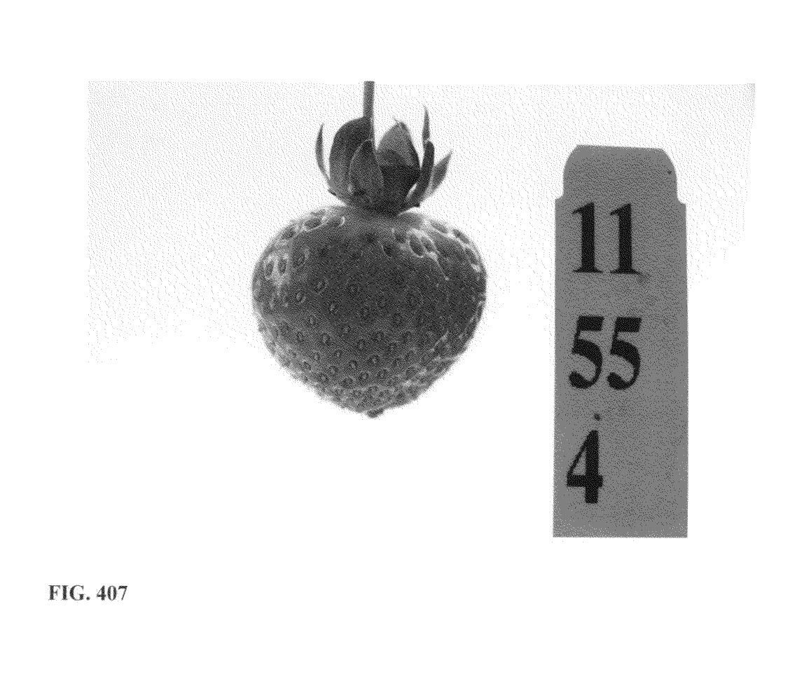

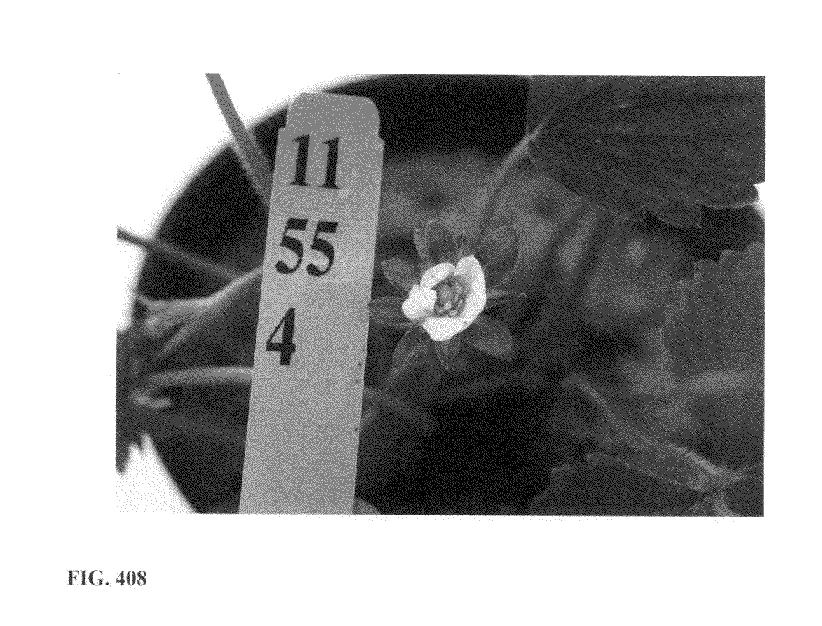

Cultivar ‘11.55-4’ has distinct disease/pathogen resistance, yield, appearance, size, and firmness. The plant of cultivar ‘11.55-4’ is characterized and distinguished in FIG. 406, the fruit of cultivar ‘11.55-4’ is characterized and distinguished in FIG. 407, and the flower of cultivar ‘11.55-4’ is characterized and distinguished in FIG. 408.

The following detailed description of cultivar ‘11.55-4’ is based on observations taken of plants and fruits (see FIGS. 406 and 407).

Certain characteristics of cultivar ‘11.55-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 136 provides data that characterizes the yield, appearance, size, and firmness of cultivar ‘11.55-4’.

TABLE 136

| Cultivar | Yield | App | Size | Firm |
|---|---|---|---|---|
| 11.55-4 | 2802 | 3.8 | 33.5 | 10.3 |

### Cultivar ‘11.57-1’

In certain aspects, the present invention relates to a new and distinctive short-day type strawberry cultivar designated as ‘11.57-1’. Cultivar ‘11.57-1’ is typical of short-day strawberry cultivars and produces fruit over an extended period when treated appropriately in arid, subtropical climates.

The strawberry cultivar ‘11.57-1’ originated from a cross between two unreleased germplasm accessions ‘08.182-2’ and ‘08.138-2’. The cultivar has been asexually reproduced by runners. The properties of this cultivar were found to be transmissible by such asexual reproduction. The cultivar is stable and reproduces true to type in successive generations of asexual reproduction.

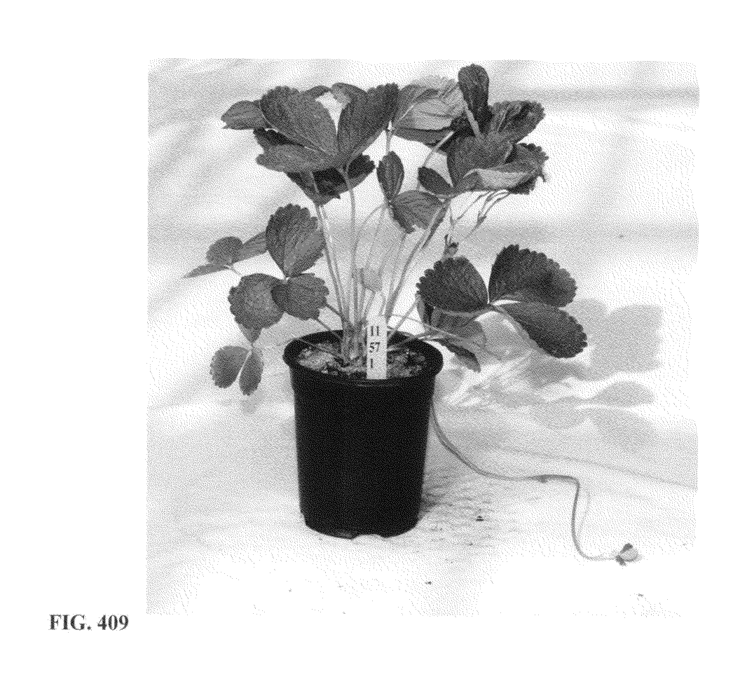

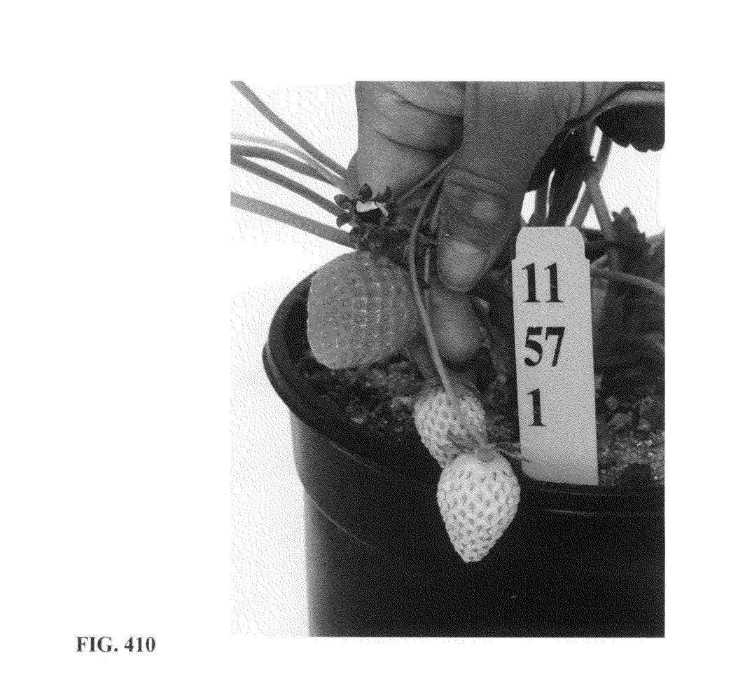

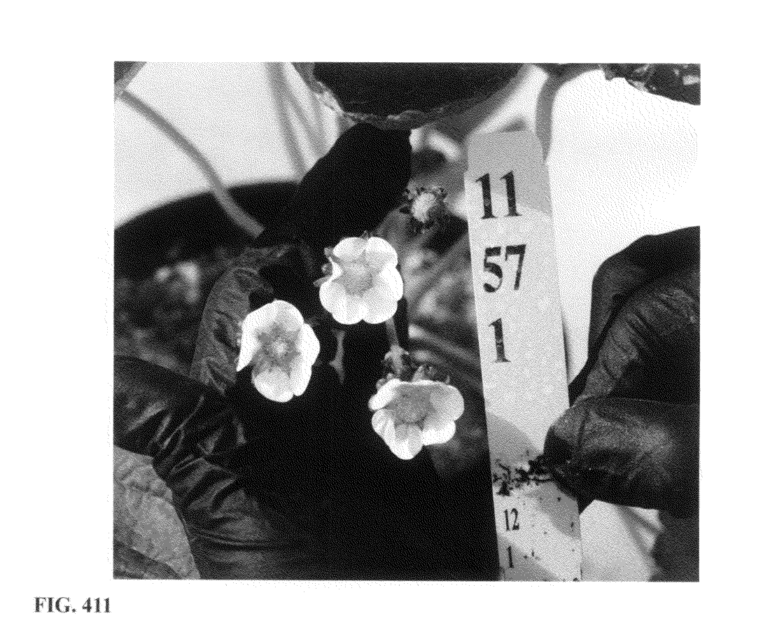

Cultivar ‘11.57-1’ has distinct disease/pathogen resistance, yield, appearance, size, and firmness. The plant of cultivar ‘11.57-1’ is characterized and distinguished in FIG. 409, the fruit of cultivar ‘11.57-1’ is characterized and distinguished in FIG. 410, and the flower of cultivar ‘11.57-1’ is characterized and distinguished in FIG. 411.

The following detailed description of cultivar ‘11.57-1’ is based on observations taken of plants and fruits (see FIGS. 409 and 410).

Certain characteristics of cultivar ‘11.57-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 137 provides data that characterizes the yield, appearance, size, and firmness of cultivar ‘11.57-1’.

TABLE 137

| Cultivar | Yield | App | Size | Firm |
|---|---|---|---|---|
| 11.57-1 | 2292 | 3.5 | 33.3 | 12.2 |

### Cultivar ‘11.62-603’

In certain aspects, the present invention relates to a new and distinctive short-day type strawberry cultivar designated as ‘11.62-603’. Cultivar ‘11.62-603’ is typical of short-day strawberry cultivars and produces fruit over an extended period when treated appropriately in arid, subtropical climates.

The strawberry cultivar ‘11.62-603’ originated from a cross between two unreleased germplasm accessions ‘08.17-5’ and ‘06.66-603’. The cultivar has been asexually reproduced by runners. The properties of this cultivar were found to be transmissible by such asexual reproduction. The cultivar is stable and reproduces true to type in successive generations of asexual reproduction.

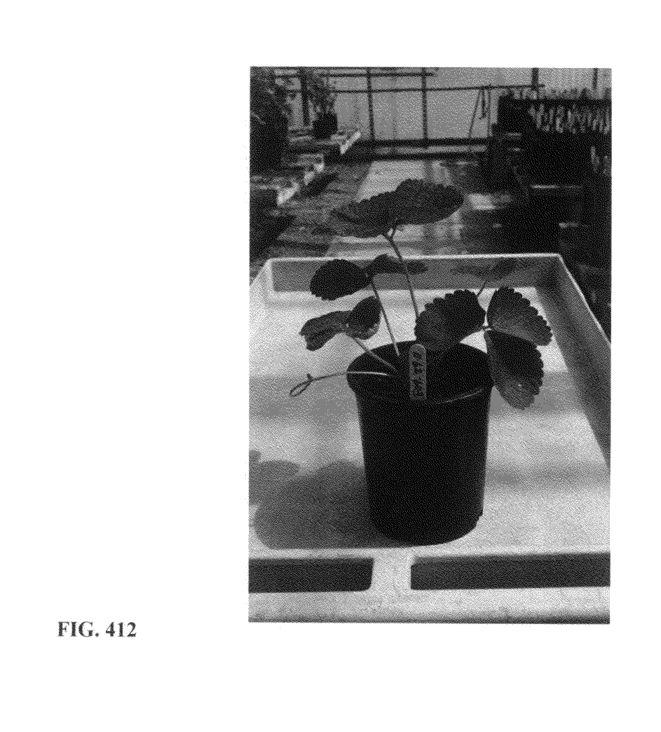

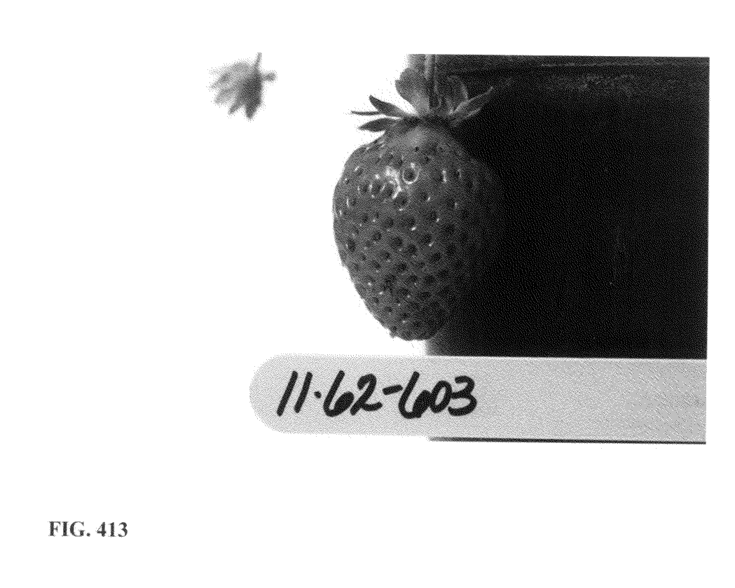

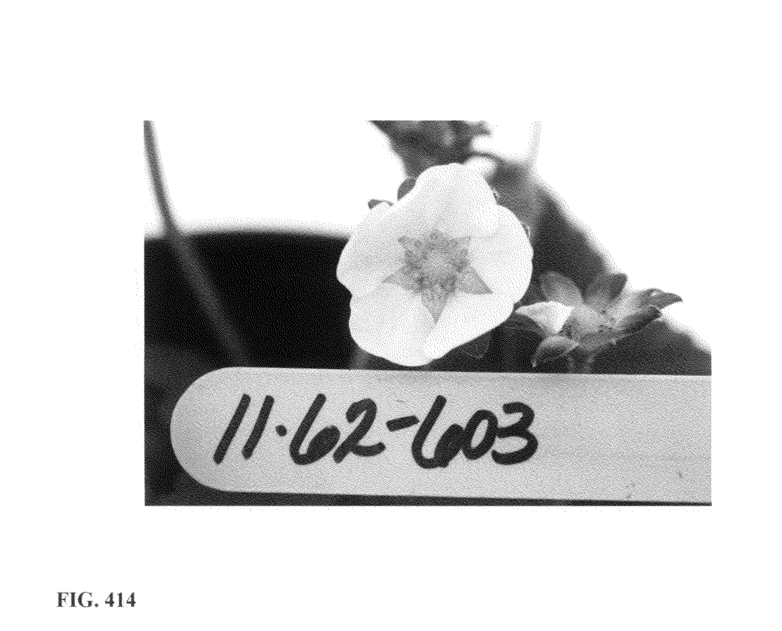

Cultivar ‘11.62-603’ has distinct disease/pathogen resistance, yield, appearance, size, and firmness. The plant of cultivar ‘11.62-603’ is characterized and distinguished in FIG. 412, the fruit of cultivar ‘11.62-603’ is characterized and distinguished in FIG. 413, and the flower of cultivar ‘11.62-603’ is characterized and distinguished in FIG. 414.

The following detailed description of cultivar ‘11.62-603’ is based on observations taken of plants and fruits (see FIGS. 412 and 413).

Certain characteristics of cultivar ‘11.62-603’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 138 provides data that characterizes the yield, appearance, size, and firmness of cultivar ‘11.62-603’.

TABLE 138

| Cultivar | Yield | App | Size | Firm |
|---|---|---|---|---|
| 11.62-603 | 1011 | 3.7 | 42.0 | 12.0 |

### Cultivar ‘11.67-1’

In certain aspects, the present invention relates to a new and distinctive short-day type strawberry cultivar designated as ‘11.67-1’. Cultivar ‘11.67-1’ is typical of short-day strawberry cultivars and produces fruit over an extended period when treated appropriately in arid, subtropical climates.

The strawberry cultivar ‘11.67-1’ originated from a cross between two unreleased germplasm accessions ‘07.50-601’ and ‘06.106-2’. The cultivar has been asexually reproduced by runners. The properties of this cultivar were found to be transmissible by such asexual reproduction. The cultivar is stable and reproduces true to type in successive generations of asexual reproduction.

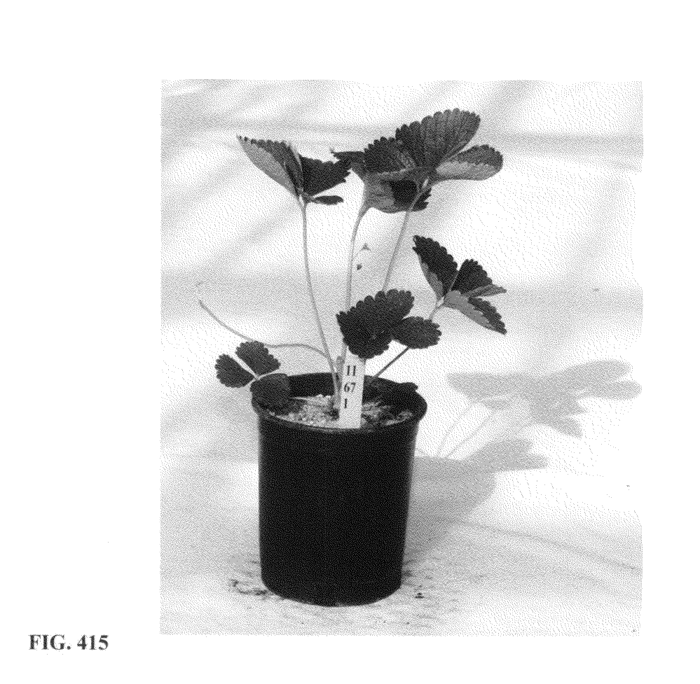

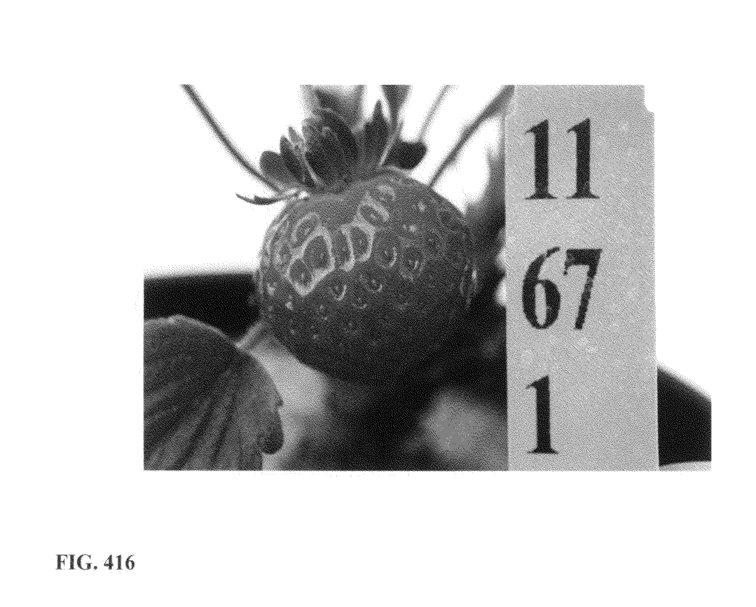

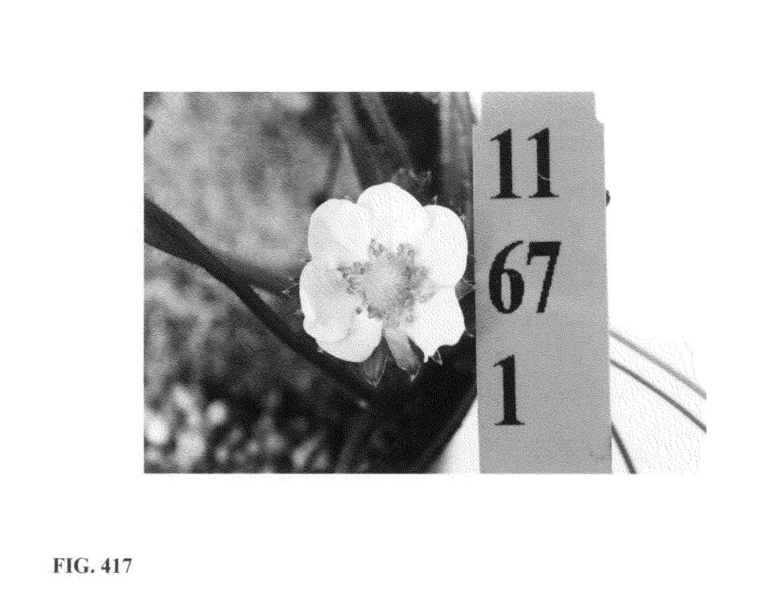

Cultivar ‘11.67-1’ has distinct disease/pathogen resistance, yield, appearance, size, and firmness. The plant of cultivar ‘11.67-1’ is characterized and distinguished in FIG. 415, the fruit of cultivar ‘11.67-1’ is characterized and distinguished in FIG. 416, and the flower of cultivar ‘11.67-1’ is characterized and distinguished in FIG. 417.

The following detailed description of cultivar ‘11.67-1’ is based on observations taken of plants and fruits (see FIGS. 415 and 416).

Certain characteristics of cultivar ‘11.67-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 139 provides data that characterizes the yield, appearance, size, and firmness of cultivar ‘11.67-1’.

TABLE 139

| Cultivar | Yield | App | Size | Firm |
|---|---|---|---|---|
| 11.67-1 | 3033 | 3.9 | 33.8 | 11.5 |

### Cultivar ‘11.71-601’

In certain aspects, the present invention relates to a new and distinctive short-day type strawberry cultivar designated as ‘11.71-601’. Cultivar ‘11.71-601’ is typical of short-day strawberry cultivars and produces fruit over an extended period when treated appropriately in arid, subtropical climates.

The strawberry cultivar ‘11.71-601’ originated from a cross between two unreleased germplasm accessions ‘07.9-601’ and ‘06.137-2’. The cultivar has been asexually reproduced by runners. The properties of this cultivar were found to be transmissible by such asexual reproduction. The cultivar is stable and reproduces true to type in successive generations of asexual reproduction.

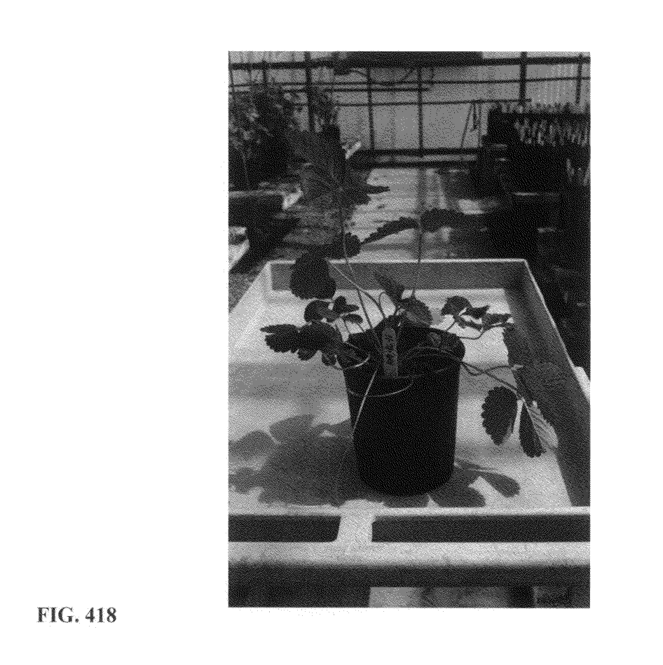

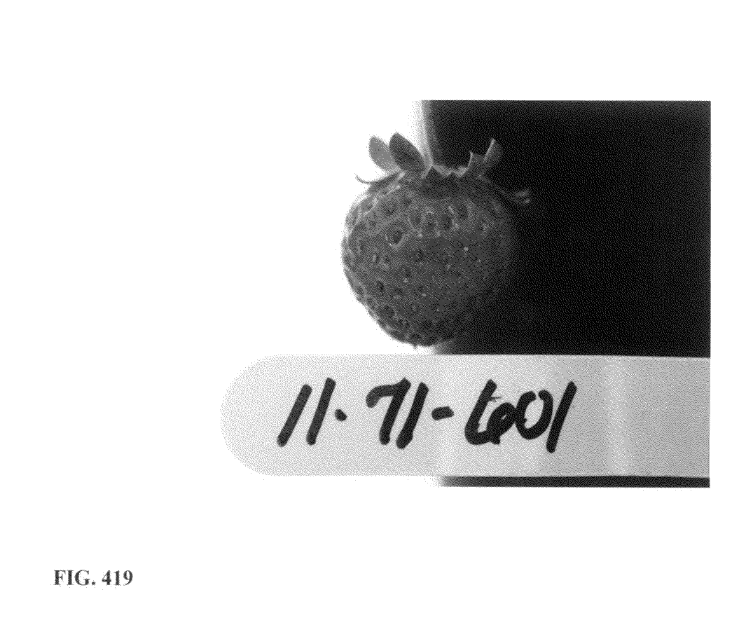

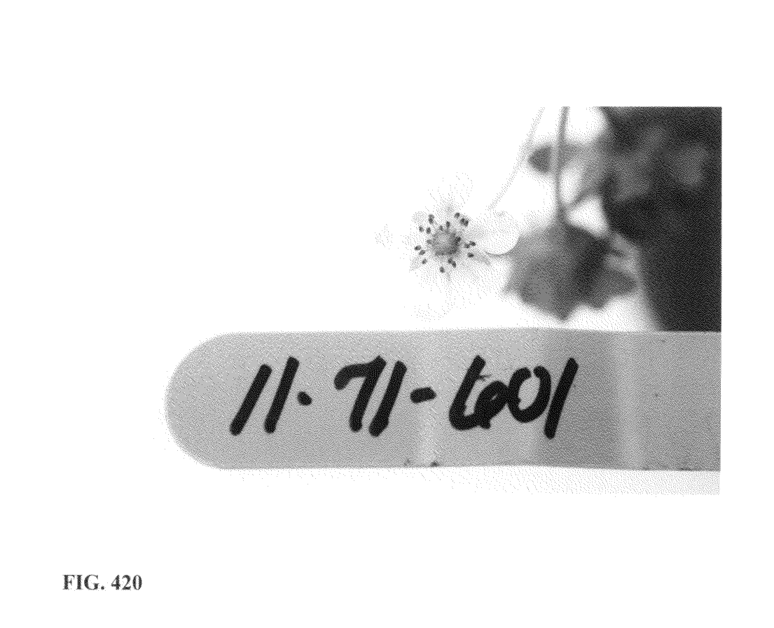

Cultivar ‘11.71-601’ has distinct disease/pathogen resistance, yield, appearance, size, and firmness. The plant of cultivar ‘11.71-601’ is characterized and distinguished in FIG. 418, the fruit of cultivar ‘11.71-601’ is characterized and distinguished in FIG. 419, and the flower of cultivar ‘11.71-601’ is characterized and distinguished in FIG. 420.

The following detailed description of cultivar ‘11.71-601’ is based on observations taken of plants and fruits (see FIGS. 418 and 419).

Certain characteristics of cultivar ‘11.71-601’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 140 provides data that characterizes the yield, appearance, size, and firmness of cultivar ‘11.71-601’.

TABLE 140

| Cultivar | Yield | App | Size | Firm |
|---|---|---|---|---|
| 11.71-601 | 1043 | 3.6 | 52.0 | 10.9 |

### Cultivar ‘11.80-2’

In certain aspects, the present invention relates to a new and distinctive day-neutral type strawberry cultivar designated as ‘11.80-2’. Cultivar ‘11.80-2’ is typical of day-neutral strawberry cultivars and produces fruit regardless of day length when treated appropriately in arid, subtropical climates.

The strawberry cultivar ‘11.80-2’ originated from a cross between two unreleased germplasm accessions ‘08.17-5’ and ‘07.43-1’. The cultivar has been asexually reproduced by runners. The properties of this cultivar were found to be transmissible by such asexual reproduction. The cultivar is stable and reproduces true to type in successive generations of asexual reproduction.

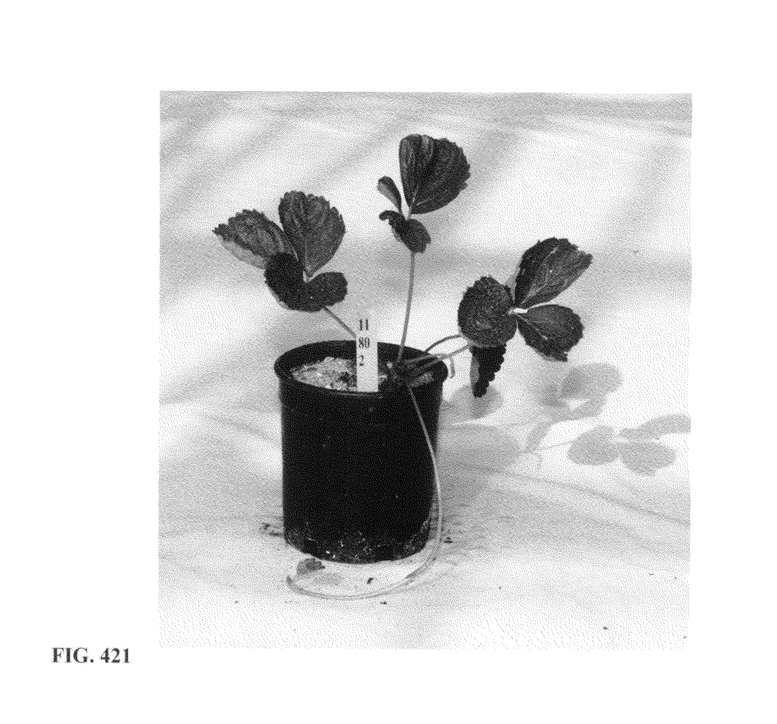

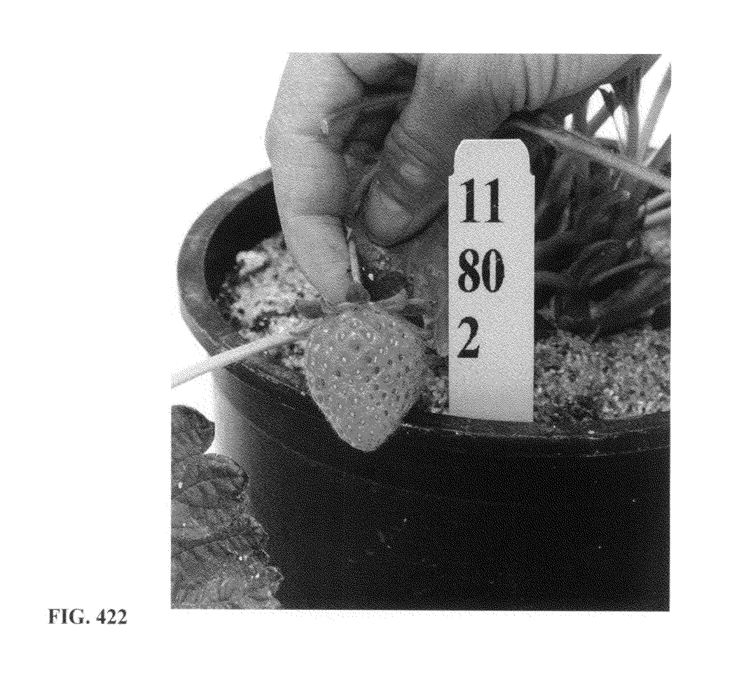

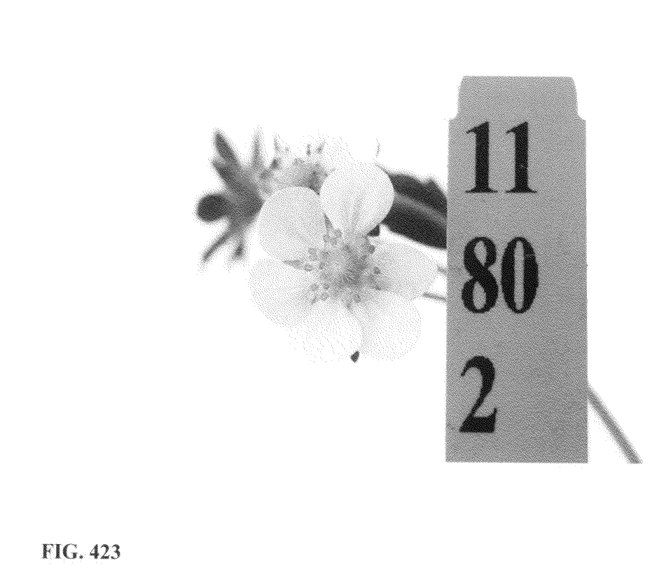

Cultivar ‘11.80-2’ has distinct disease/pathogen resistance, yield, appearance, size, and firmness. The plant of cultivar ‘11.80-2’ is characterized and distinguished in FIG. 421, the fruit of cultivar ‘11.80-2’ is characterized and distinguished in FIG. 422, and the flower of cultivar ‘11.80-2’ is characterized and distinguished in FIG. 423.

The following detailed description of cultivar ‘11.80-2’ is based on observations taken of plants and fruits (see FIGS. 421 and 422).

Certain characteristics of cultivar ‘11.80-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 141 provides data that characterizes the yield, appearance, size, and firmness of cultivar ‘11.80-2’.

TABLE 141

| Cultivar | Yield | App | Size | Firm |
|---|---|---|---|---|
| 11.80-2 | 2247 | 4.0 | 38.3 | 12.7 |

### Cultivar ‘11.103-1’

In certain aspects, the present invention relates to a new and distinctive day-neutral type strawberry cultivar designated as ‘11.103-1’. Cultivar ‘11.103-1’ is typical of day-neutral strawberry cultivars and produces fruit regardless of day length when treated appropriately in arid, subtropical climates.

The strawberry cultivar ‘11.103-1’ originated from a cross between unreleased germplasm accession ‘07.92-3’ and cultivar ‘Merced’. The cultivar has been asexually reproduced by runners. The properties of this cultivar were found to be transmissible by such asexual reproduction. The cultivar is stable and reproduces true to type in successive generations of asexual reproduction.

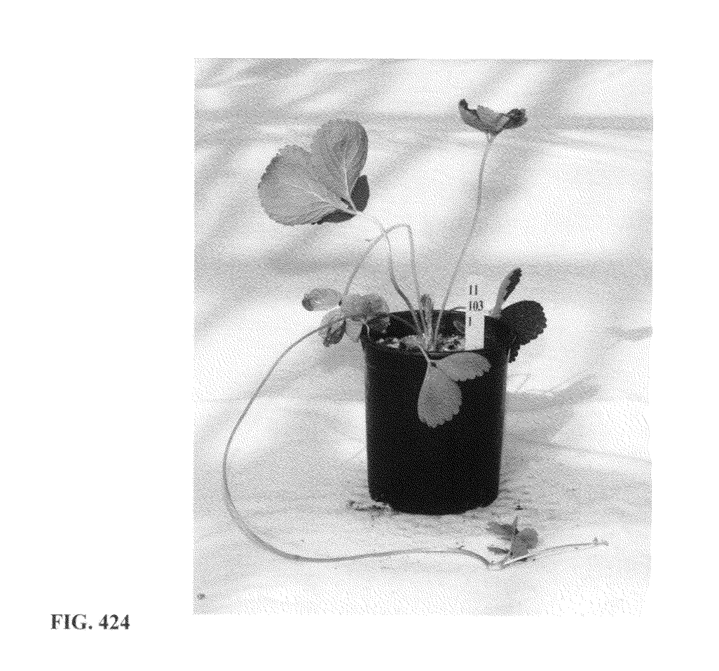

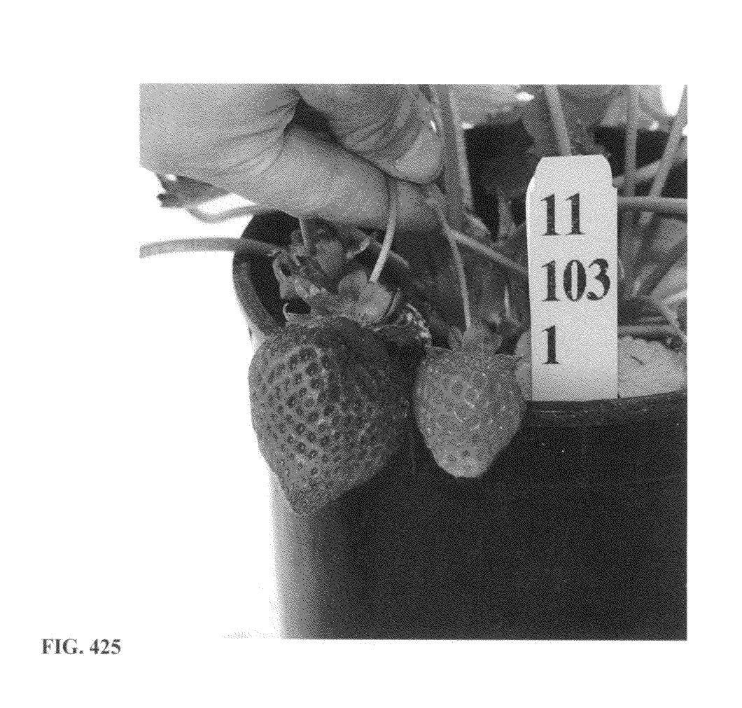

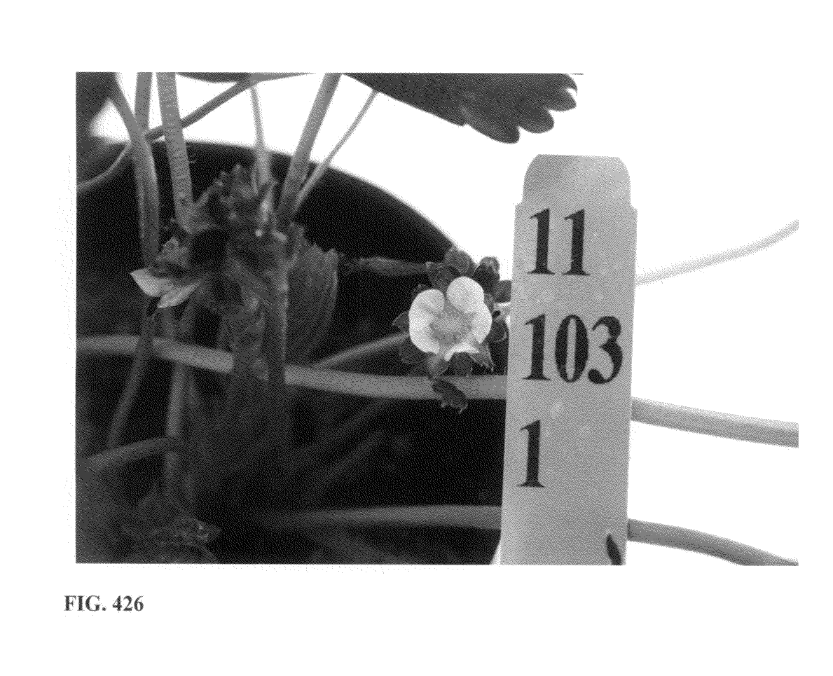

Cultivar ‘11.103-1’ has distinct disease/pathogen resistance, yield, appearance, size, and firmness. The plant of cultivar ‘11.103-1’ is characterized and distinguished in FIG. 424, the fruit of cultivar ‘11.103-1’ is characterized and distinguished in FIG. 425, and the flower of cultivar ‘11.103-1’ is characterized and distinguished in FIG. 426.

The following detailed description of cultivar ‘11.103-1’ is based on observations taken of plants and fruits (see FIGS. 424 and 425).

Certain characteristics of cultivar ‘11.103-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 142 provides data that characterizes the yield, appearance, size, and firmness of cultivar ‘11.103-1’.

TABLE 142

| Cultivar | Yield | App | Size | Firm |
|---|---|---|---|---|
| 11.103-1 | 3105 | 4.2 | 34.4 | 12.6 |

### Cultivar ‘11.103-3’

In certain aspects, the present invention relates to a new and distinctive short-day type strawberry cultivar designated as ‘11.103-3’. Cultivar ‘11.103-3’ is typical of short-day strawberry cultivars and produces fruit over an extended period when treated appropriately in arid, subtropical climates.

The strawberry cultivar ‘11.103-3’ originated from a cross between unreleased germplasm accession ‘07.92-3’ and cultivar ‘Merced’. The cultivar has been asexually reproduced by runners. The properties of this cultivar were found to be transmissible by such asexual reproduction. The cultivar is stable and reproduces true to type in successive generations of asexual reproduction.

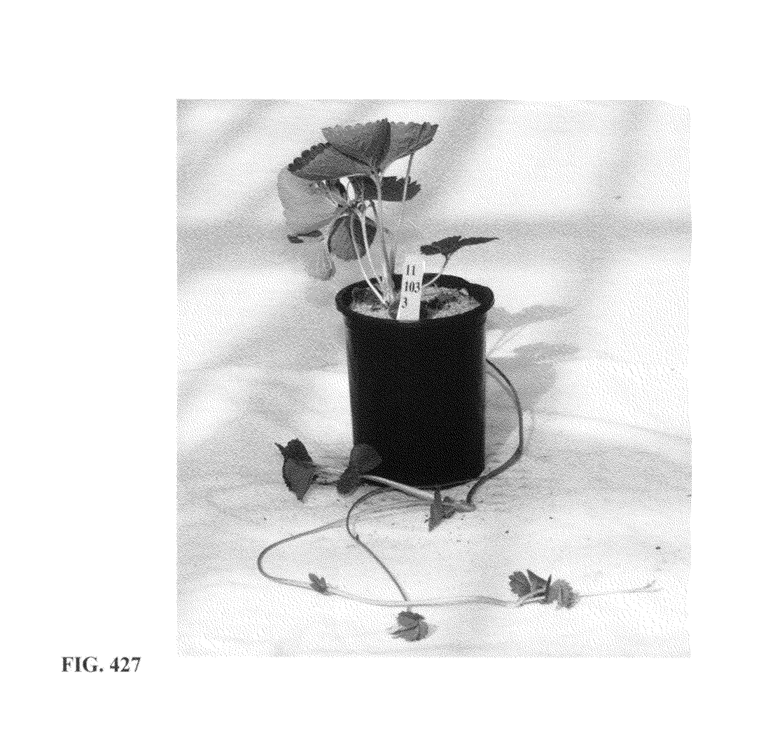

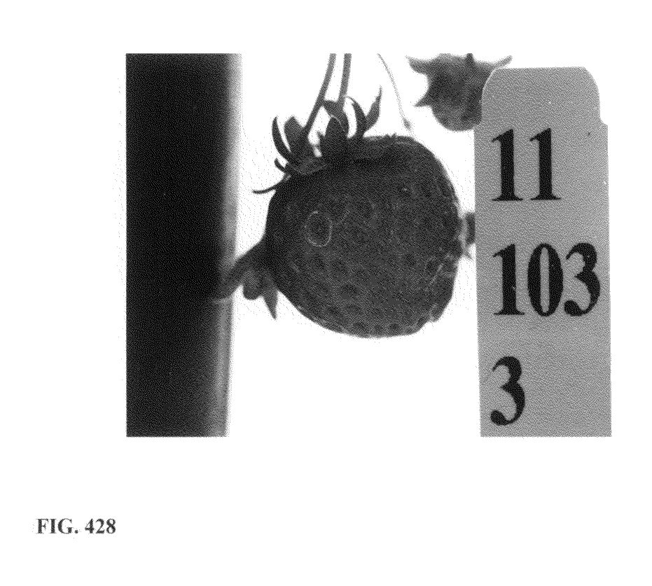

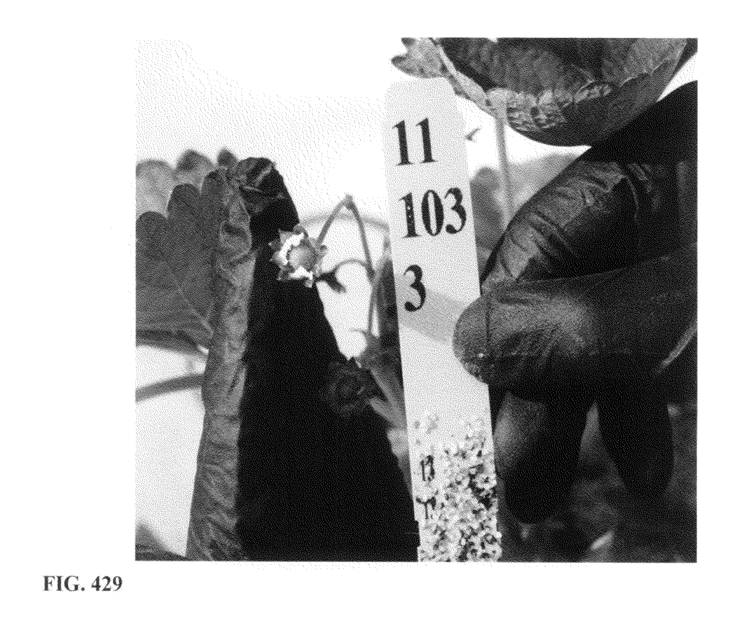

Cultivar ‘11.103-3’ has distinct disease/pathogen resistance, yield, appearance, size, and firmness. The plant of cultivar ‘11.103-3’ is characterized and distinguished in FIG. 427, the fruit of cultivar ‘11.103-3’ is characterized and distinguished in FIG. 428, and the flower of cultivar ‘11.103-3’ is characterized and distinguished in FIG. 429.

The following detailed description of cultivar ‘11.103-3’ is based on observations taken of plants and fruits (see FIGS. 427 and 428).

Certain characteristics of cultivar ‘11.103-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 143 provides data that characterizes the yield, appearance, size, and firmness of cultivar ‘11.103-3’.

TABLE 143

| Cultivar | Yield | App | Size | Firm |
|---|---|---|---|---|
| 11.103-3 | 1786 | 3.8 | 36.4 | 12.8 |

### Cultivar ‘11.103-5’

In certain aspects, the present invention relates to a new and distinctive short-day type strawberry cultivar designated as ‘11.103-5’. Cultivar ‘11.103-5’ is typical of short-day strawberry cultivars and produces fruit over an extended period when treated appropriately in arid, subtropical climates.

The strawberry cultivar ‘11.103-5’ originated from a cross between unreleased germplasm accession ‘07.92-3’ and cultivar ‘Merced’. The cultivar has been asexually reproduced by runners. The properties of this cultivar were found to be transmissible by such asexual reproduction. The cultivar is stable and reproduces true to type in successive generations of asexual reproduction.

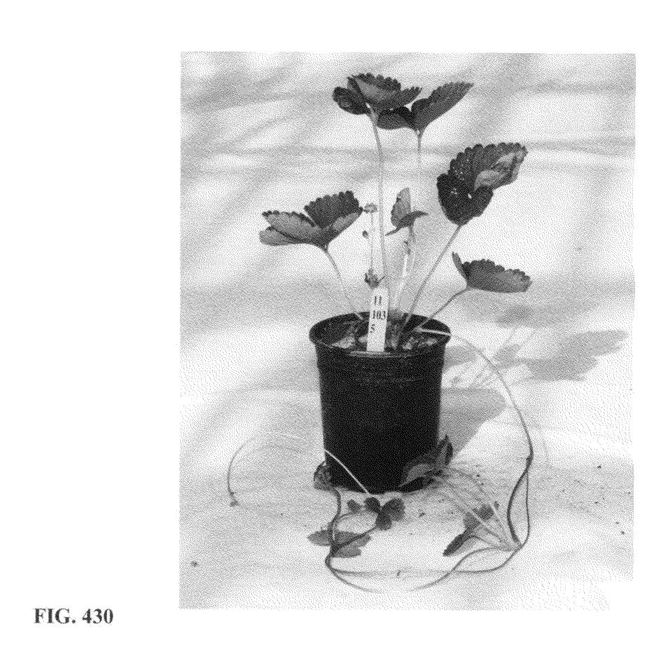

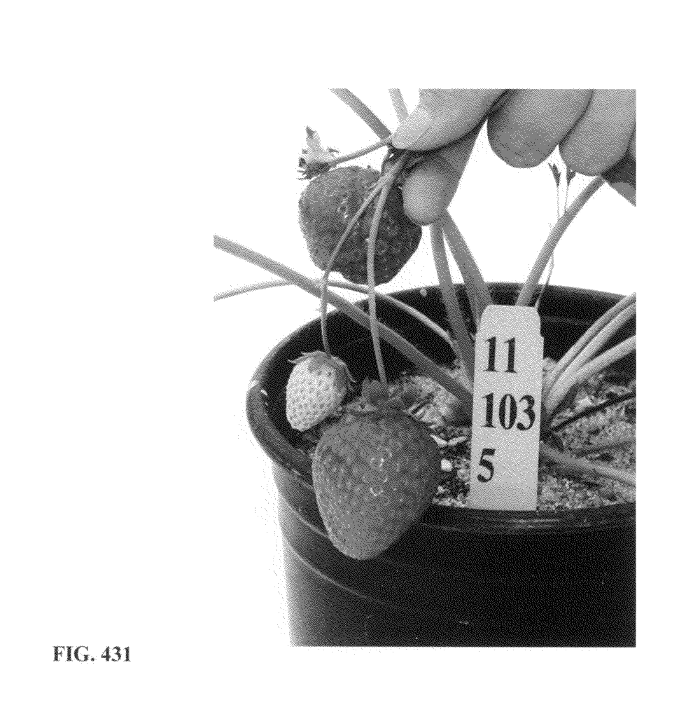

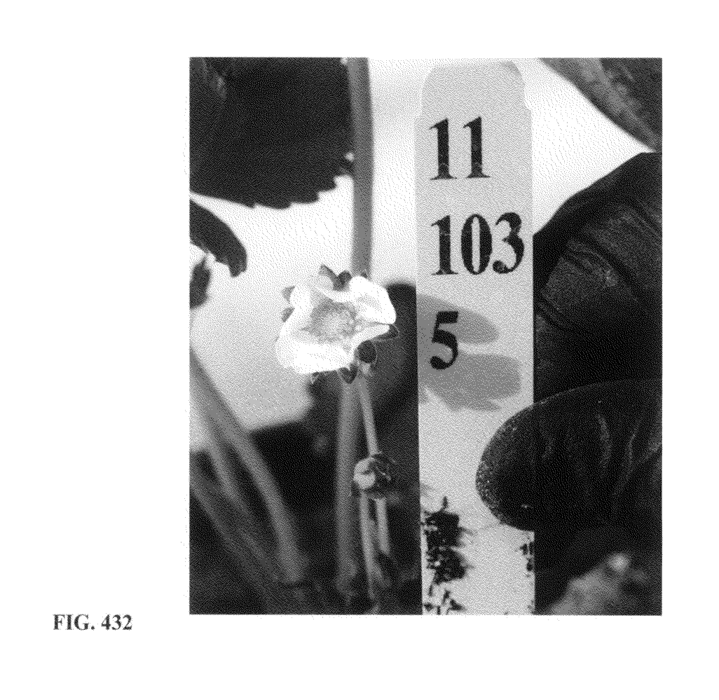

Cultivar ‘11.103-5’ has distinct disease/pathogen resistance, yield, appearance, size, and firmness. The plant of cultivar ‘11.103-5’ is characterized and distinguished in FIG. 430, the fruit of cultivar ‘11.103-5’ is characterized and distinguished in FIG. 431, and the flower of cultivar ‘11.103-5’ is characterized and distinguished in FIG. 432.

The following detailed description of cultivar ‘11.103-5’ is based on observations taken of plants and fruits (see FIGS. 430 and 431).

Certain characteristics of cultivar ‘11.103-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 144 provides data that characterizes the yield, appearance, size, and firmness of cultivar ‘11.103-5’.

TABLE 144

| Cultivar | Yield | App | Size | Firm |
|---|---|---|---|---|
| 11.103-5 | 2169 | 3.9 | 31.8 | 10.8 |

### Cultivar ‘11.105-1’

In certain aspects, the present invention relates to a new and distinctive day-neutral type strawberry cultivar designated as ‘11.105-1’. Cultivar ‘11.105-1’ is typical of day-neutral strawberry cultivars and produces fruit regardless of day length when treated appropriately in arid, subtropical climates.

The strawberry cultivar ‘11.105-1’ originated from a cross between two unreleased germplasm accessions ‘07.64-2’ and ‘08.75-1’. The cultivar has been asexually reproduced by runners. The properties of this cultivar were found to be transmissible by such asexual reproduction. The cultivar is stable and reproduces true to type in successive generations of asexual reproduction.

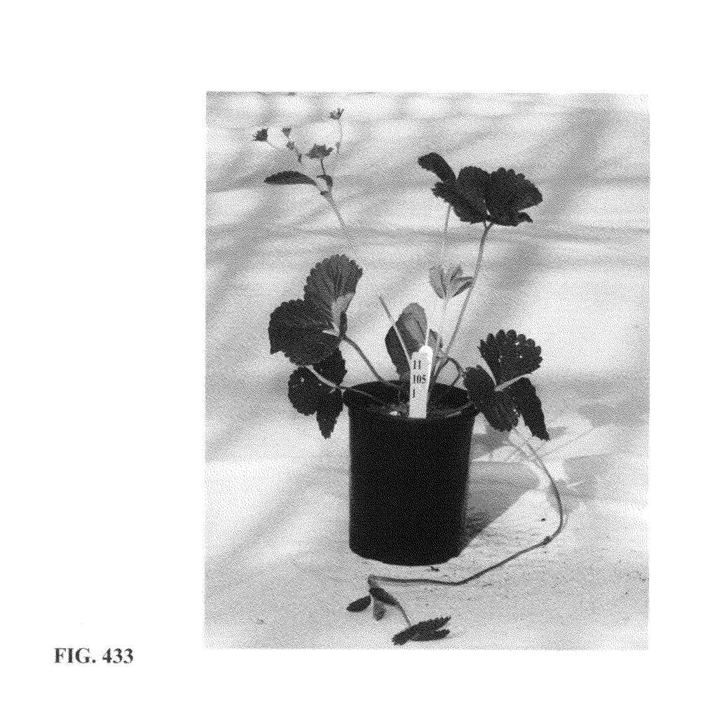

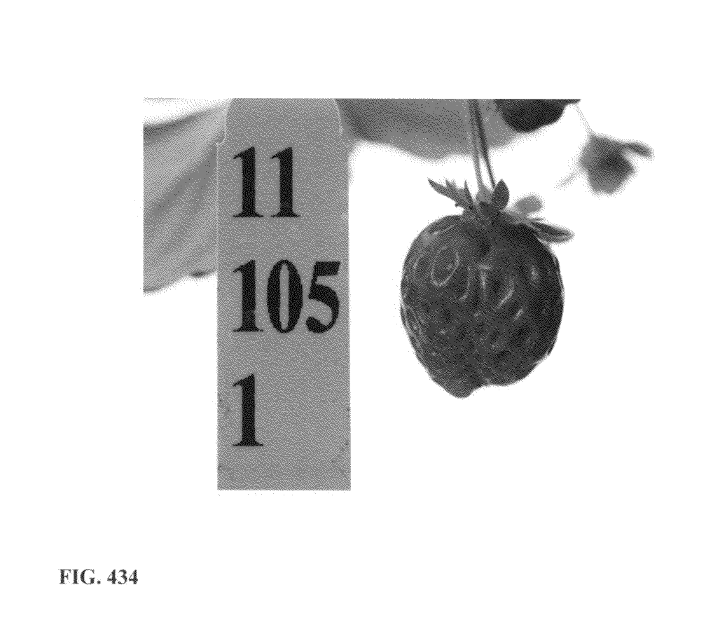

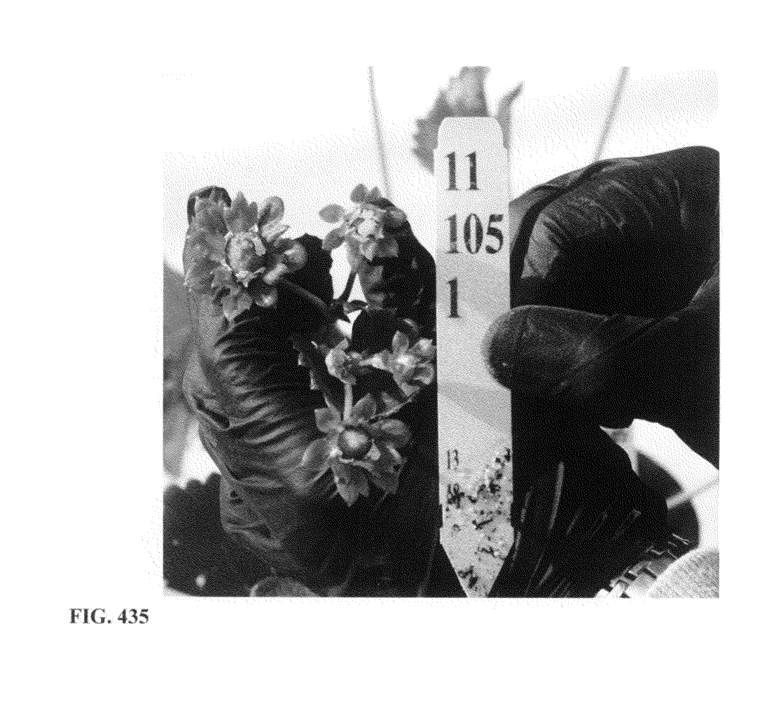

Cultivar ‘11.105-1’ has distinct disease/pathogen resistance, yield, appearance, size, and firmness. The plant of cultivar ‘11.105-1’ is characterized and distinguished in FIG. 433, the fruit of cultivar ‘11.105-1’ is characterized and distinguished in FIG. 434, and the flower of cultivar ‘11.105-1’ is characterized and distinguished in FIG. 435.

The following detailed description of cultivar ‘11.105-1’ is based on observations taken of plants and fruits (see FIGS. 433 and 434).

Certain characteristics of cultivar ‘11.105-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 145 provides data that characterizes the yield, appearance, size, and firmness of cultivar ‘11.105-1’.

TABLE 145

| Cultivar | Yield | App | Size | Firm |
|---|---|---|---|---|
| 11.105-1 | 2235 | 3.9 | 30.3 | 11.2 |

### Cultivar ‘11.107-5’

In certain aspects, the present invention relates to a new and distinctive short-day type strawberry cultivar designated as ‘11.107-5’. Cultivar ‘11.107-5’ is typical of short-day strawberry cultivars and produces fruit over an extended period when treated appropriately in arid, subtropical climates.

The strawberry cultivar ‘11.107-5’ originated from a cross between cultivar ‘Merced’ and unreleased germplasm accession ‘08.75-1’. The cultivar has been asexually reproduced by runners. The properties of this cultivar were found to be transmissible by such asexual reproduction. The cultivar is stable and reproduces true to type in successive generations of asexual reproduction.

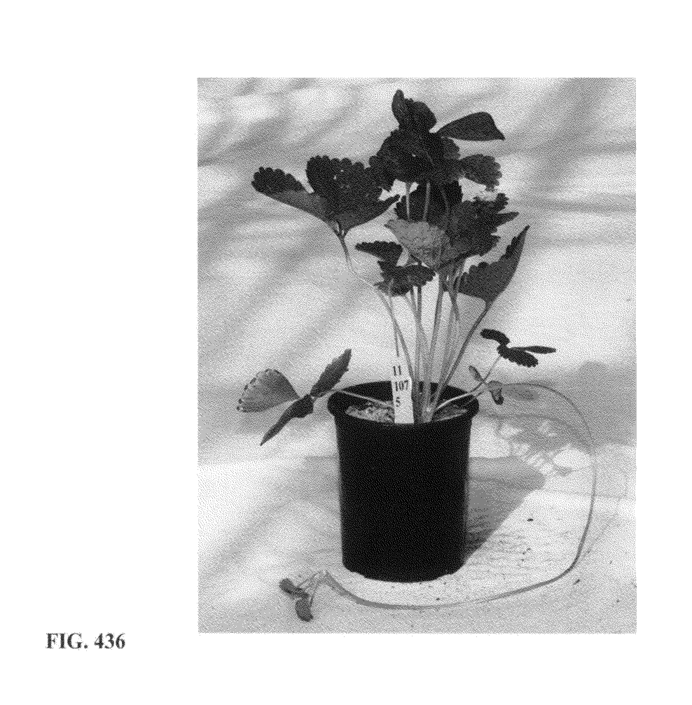

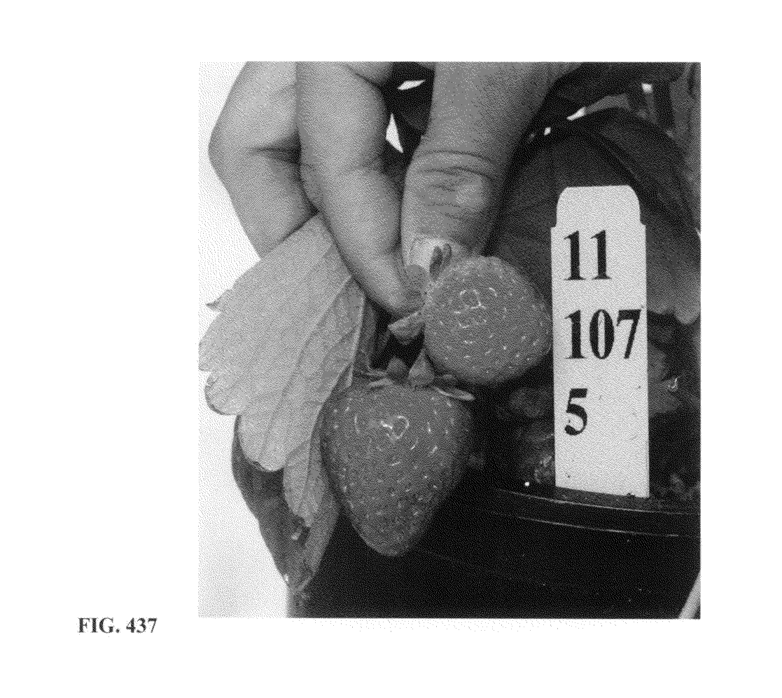

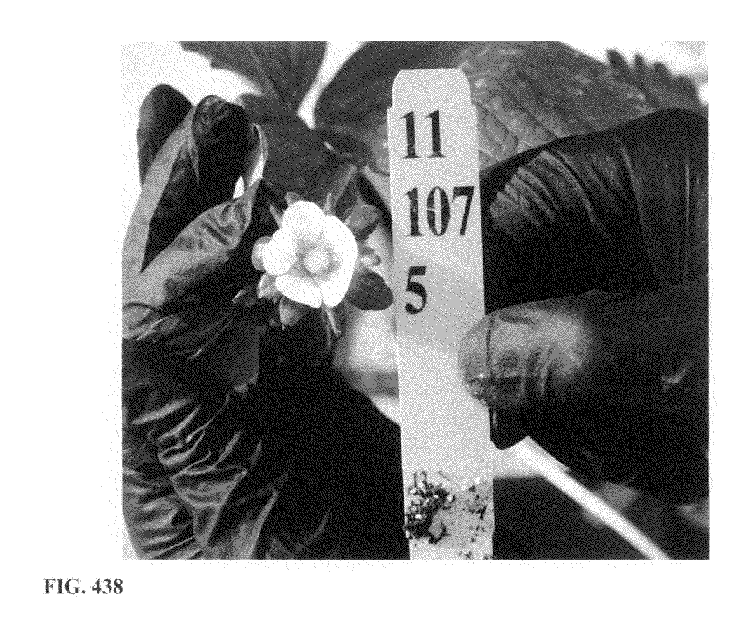

Cultivar ‘11.107-5’ has distinct disease/pathogen resistance, yield, appearance, size, and firmness. The plant of cultivar ‘11.107-5’ is characterized and distinguished in FIG. 436, the fruit of cultivar ‘11.107-5’ is characterized and distinguished in FIG. 437, and the flower of cultivar ‘11.107-5’ is characterized and distinguished in FIG. 438.

The following detailed description of cultivar ‘11.107-5’ is based on observations taken of plants and fruits (see FIGS. 436 and 437).

Certain characteristics of cultivar ‘11.107-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 146 provides data that characterizes the yield, appearance, size, and firmness of cultivar ‘11.107-5’.

TABLE 146

| Cultivar | Yield | App | Size | Firm |
|---|---|---|---|---|
| 11.107-5 | 1929 | 3.7 | 32.3 | 11.1 |

### Cultivar ‘11.109-2’

In certain aspects, the present invention relates to a new and distinctive short-day type strawberry cultivar designated as ‘11.109-2’. Cultivar ‘11.109-2’ is typical of short-day strawberry cultivars and produces fruit over an extended period when treated appropriately in arid, subtropical climates.

The strawberry cultivar ‘11.109-2’ originated from a cross between two unreleased germplasm accessions ‘07.64-2’ and ‘08.135-4’. The cultivar has been asexually reproduced by runners. The properties of this cultivar were found to be transmissible by such asexual reproduction. The cultivar is stable and reproduces true to type in successive generations of asexual reproduction.

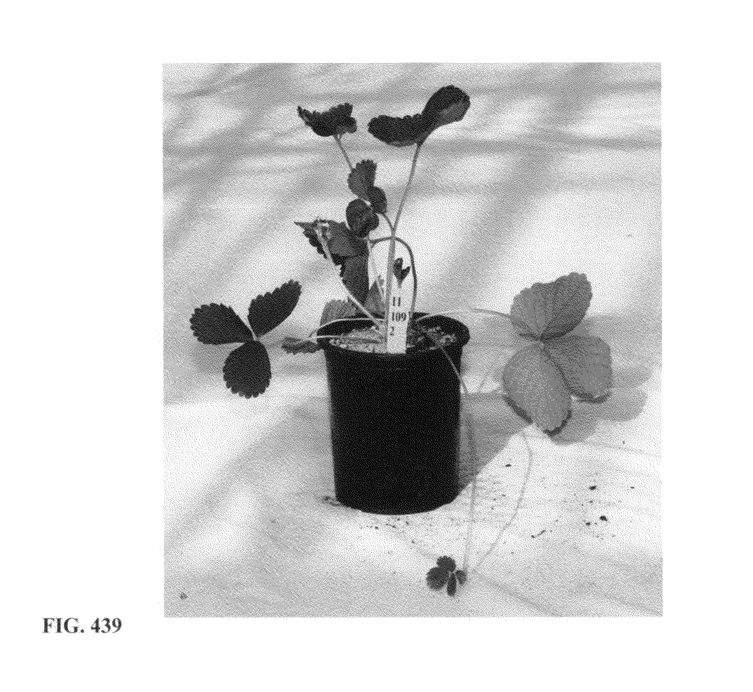

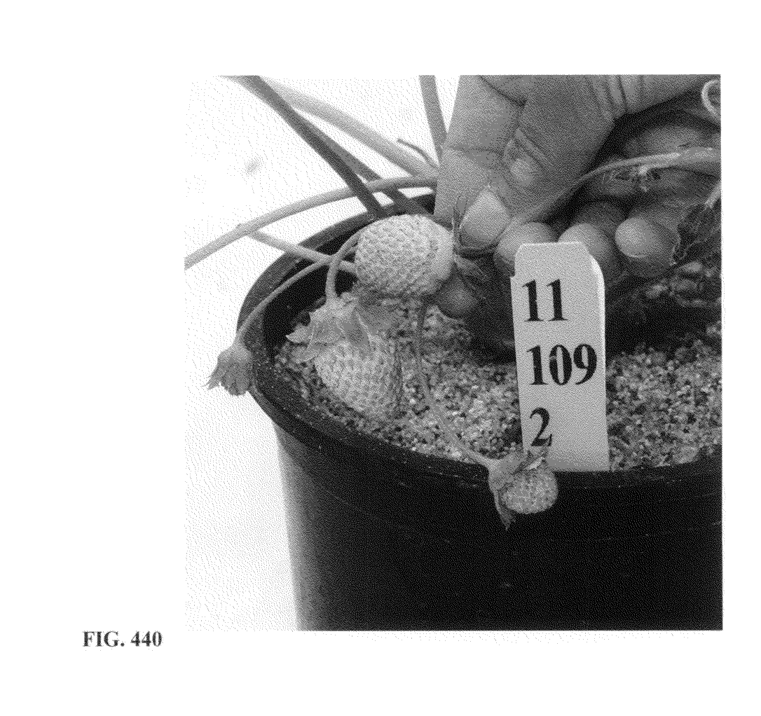

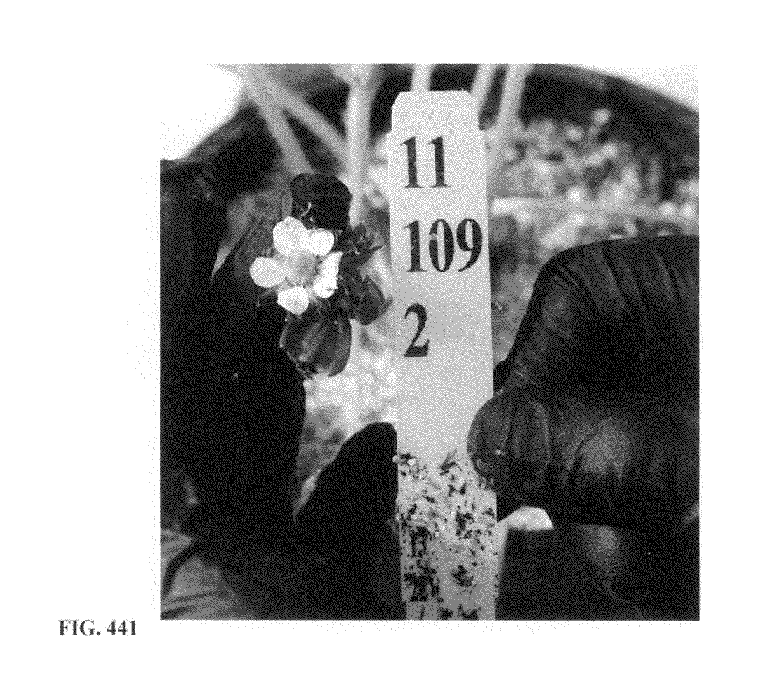

Cultivar ‘11.109-2’ has distinct disease/pathogen resistance′, yield, appearance, size, and firmness. The plant of cultivar ‘11.109-2’ is characterized and distinguished in FIG. 439, the fruit of cultivar ‘11.109-2’ is characterized and distinguished in FIG. 440, and the flower of cultivar ‘11.109-2’ is characterized and distinguished in FIG. 441.

The following detailed description of cultivar ‘11.109-2’ is based on observations taken of plants and fruits (see FIGS. 439 and 440).

Certain characteristics of cultivar ‘11.109-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 147 provides data that characterizes the yield, appearance, size, and firmness of cultivar ‘11.109-2’.

TABLE 147

| Cultivar | Yield | App | Size | Firm |
|---|---|---|---|---|
| 11.109-2 | 2242 | 4.0 | 32.5 | 10.3 |

### Cultivar ‘11.109-3’

In certain aspects, the present invention relates to a new and distinctive day-neutral type strawberry cultivar designated as ‘11.109-3’. Cultivar ‘11.109-3’ is typical of day-neutral strawberry cultivars and produces fruit regardless of day length when treated appropriately in arid, subtropical climates.

The strawberry cultivar ‘11.109-3’ originated from a cross between two unreleased germplasm accessions ‘07.64-2’ and ‘08.135-4’. The cultivar has been asexually reproduced by runners. The properties of this cultivar were found to be transmissible by such asexual reproduction. The cultivar is stable and reproduces true to type in successive generations of asexual reproduction.

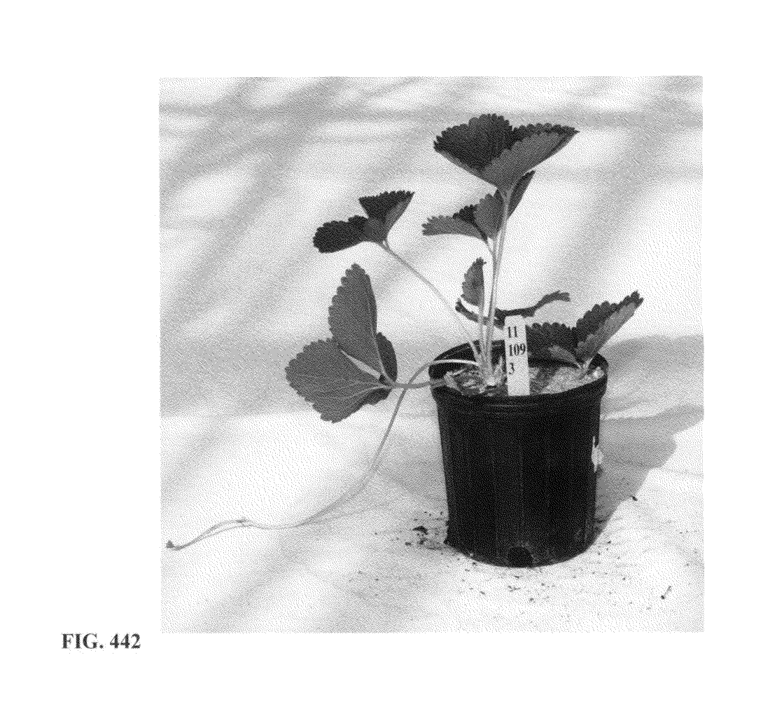

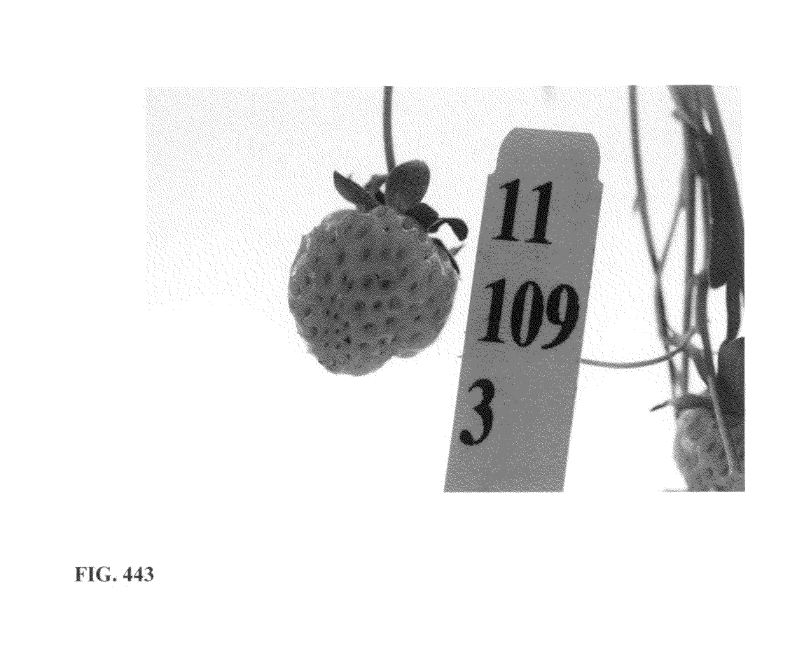

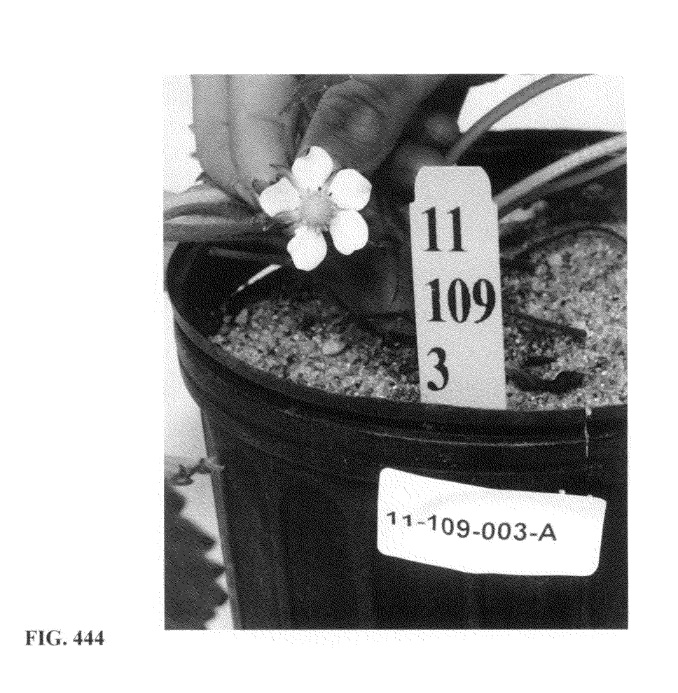

Cultivar ‘11.109-3’ has distinct disease/pathogen resistance, yield, appearance, size, and firmness. The plant of cultivar ‘11.109-3’ is characterized and distinguished in FIG. 442, the fruit of cultivar ‘11.109-3’ is characterized and distinguished in FIG. 443, and the flower of cultivar ‘11.109-3’ is characterized and distinguished in FIG. 444.

The following detailed description of cultivar ‘11.109-3’ is based on observations taken of plants and fruits (see FIGS. 442 and 443).

Certain characteristics of cultivar ‘11.109-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 148 provides data that characterizes the yield, appearance, size, and firmness of cultivar ‘11.109-3’.

TABLE 148

| Cultivar | Yield | App | Size | Firm |
|---|---|---|---|---|
| 11.109-3 | 2523 | 3.6 | 32.4 | 10.0 |

### Cultivar ‘11.110-3’

In certain aspects, the present invention relates to a new and distinctive day-neutral type strawberry cultivar designated as ‘11.110-3’. Cultivar ‘11.110-3’ is typical of day-neutral strawberry cultivars and produces fruit regardless of day length when treated appropriately in arid, subtropical climates.

The strawberry cultivar ‘11.110-3’ originated from a cross between two unreleased germplasm accessions ‘07.92-3’ and ‘08.135-4’. The cultivar has been asexually reproduced by runners. The properties of this cultivar were found to be transmissible by such asexual reproduction. The cultivar is stable and reproduces true to type in successive generations of asexual reproduction.

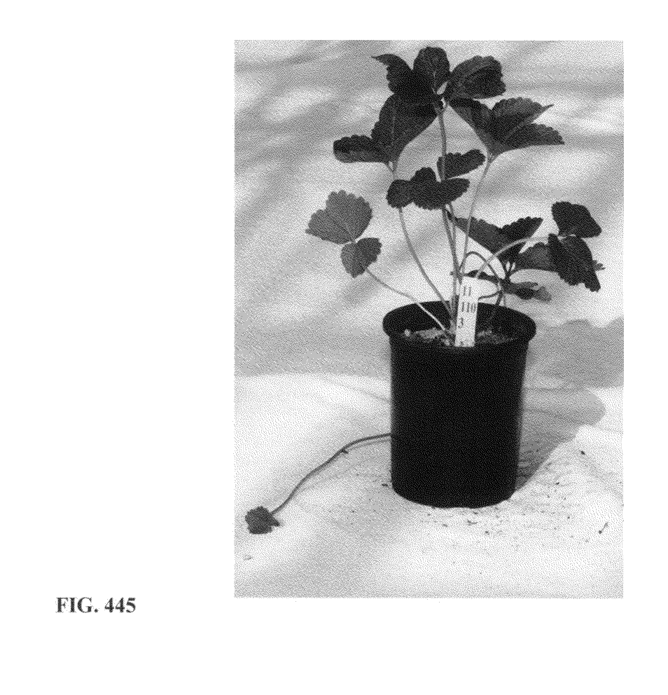

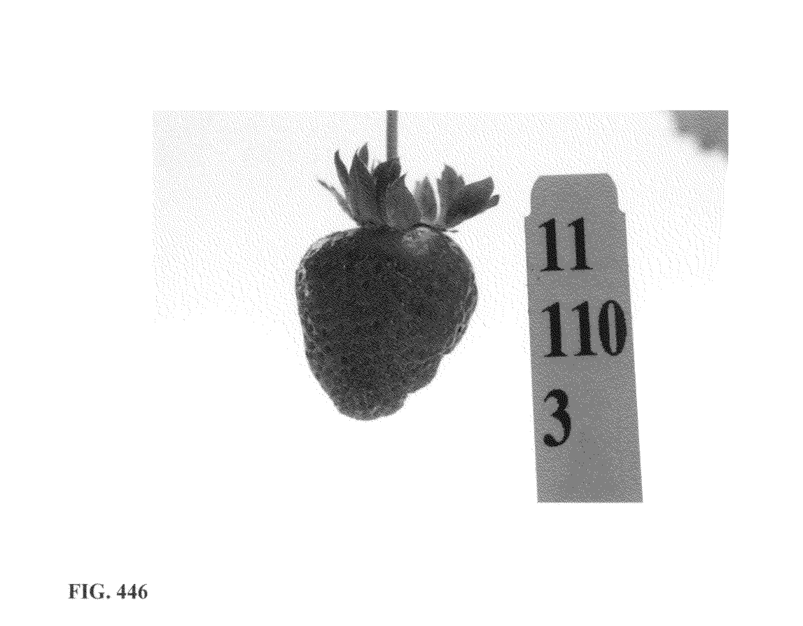

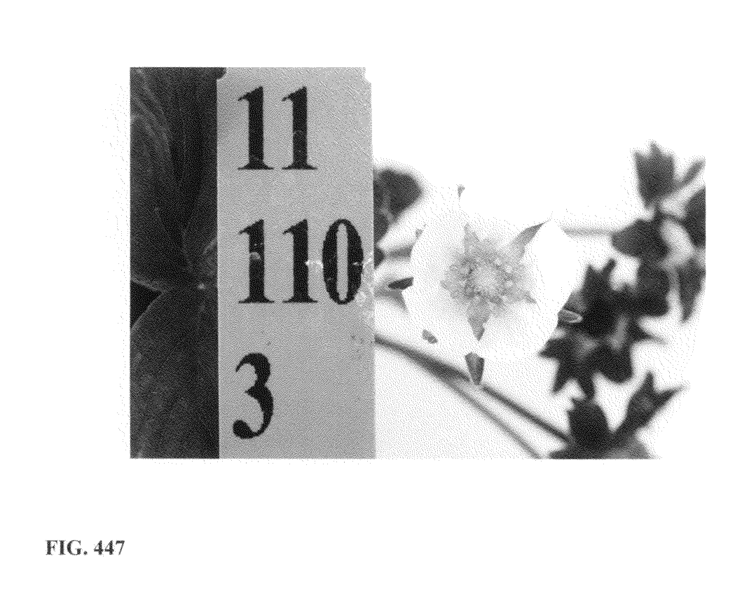

Cultivar ‘11.110-3’ has distinct disease/pathogen resistance, yield, appearance, size, and firmness. The plant of cultivar ‘11.110-3’ is characterized and distinguished in FIG. 445, the fruit of cultivar ‘11.110-3’ is characterized and distinguished in FIG. 446, and the flower of cultivar ‘11.110-3’ is characterized and distinguished in FIG. 447.

The following detailed description of cultivar ‘11.110-3’ is based on observations taken of plants and fruits (see FIGS. 445 and 446).

Certain characteristics of cultivar ‘11.110-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 149 provides data that characterizes the yield, appearance, size, and firmness of cultivar ‘11.110-3’.

TABLE 149

| Cultivar | Yield | App | Size | Firm |
|---|---|---|---|---|
| 11.110-3 | 2436 | 3.7 | 30.8 | 11.7 |

### Cultivar ‘11.113-6’

In certain aspects, the present invention relates to a new and distinctive short-day type strawberry cultivar designated as ‘11.113-6’. Cultivar ‘11.113-6’ is typical of short-day strawberry cultivars and produces fruit over an extended period when treated appropriately in arid, subtropical climates.

The strawberry cultivar ‘11.113-6’ originated from a cross between two unreleased germplasm accessions ‘06.204-1’ and ‘08.182-2’. The cultivar has been asexually reproduced by runners. The properties of this cultivar were found to be transmissible by such asexual reproduction. The cultivar is stable and reproduces true to type in successive generations of asexual reproduction.

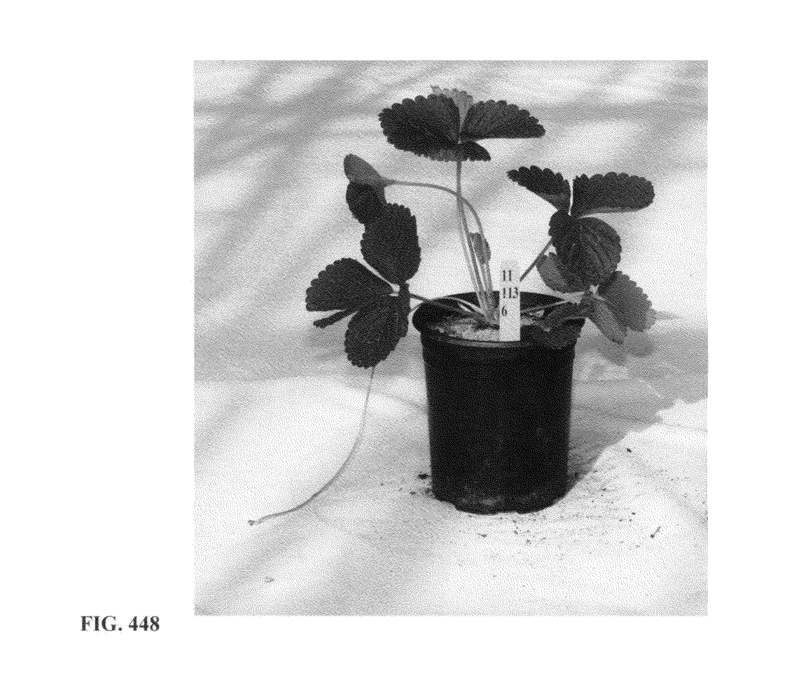

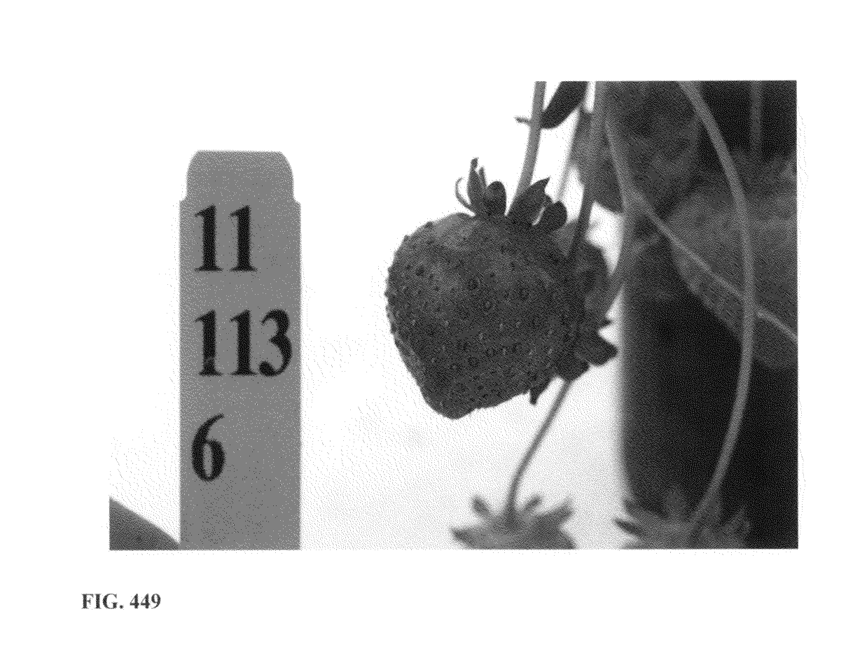

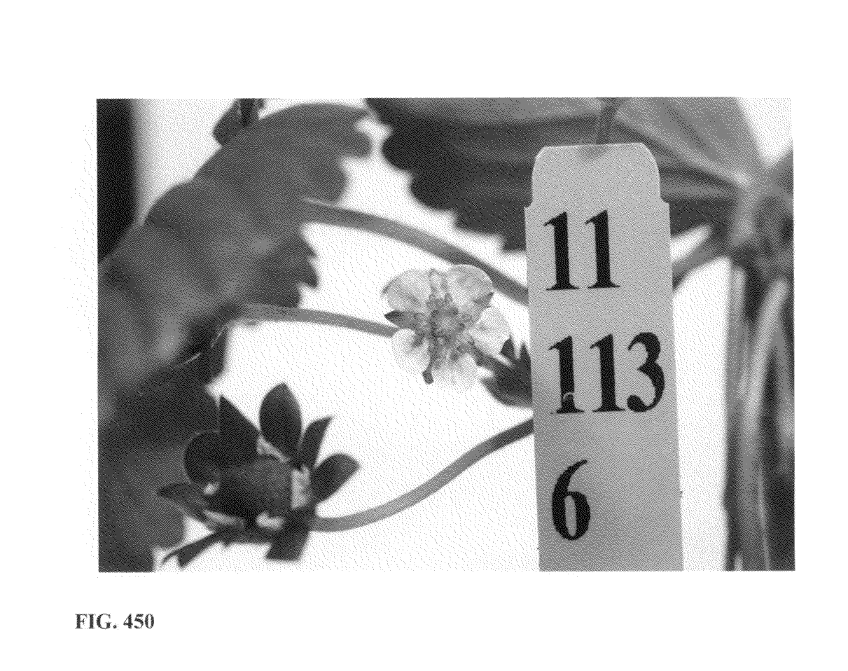

Cultivar ‘11.113-6’ has distinct disease/pathogen resistance, yield, appearance, size, and firmness. The plant of cultivar ‘11.113-6’ is characterized and distinguished in FIG. 448, the fruit of cultivar ‘11.113-6’ is characterized and distinguished in FIG. 449, and the flower of cultivar ‘11.113-6’ is characterized and distinguished in FIG. 450.

The following detailed description of cultivar ‘11.113-6’ is based on observations taken of plants and fruits (see FIGS. 448 and 449).

Certain characteristics of cultivar ‘11.113-6’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 150 provides data that characterizes the yield, appearance, size, and firmness of cultivar ‘11.113-6’.

TABLE 150

| Cultivar | Yield | App | Size | Firm |
|---|---|---|---|---|
| 11.113-6 | 3178 | 4.1 | 32.0 | 11.3 |

### Cultivar ‘11.115-3’

In certain aspects, the present invention relates to a new and distinctive day-neutral type strawberry cultivar designated as ‘11.115-3’. Cultivar ‘11.115-3’ is typical of day-neutral strawberry cultivars and produces fruit regardless of day length when treated appropriately in arid, subtropical climates.

The strawberry cultivar ‘11.115-3’ originated from a cross between two unreleased germplasm accessions ‘07.92-3’ and ‘08.182-2’. The cultivar has been asexually reproduced by runners. The properties of this cultivar were found to be transmissible by such asexual reproduction. The cultivar is stable and reproduces true to type in successive generations of asexual reproduction.

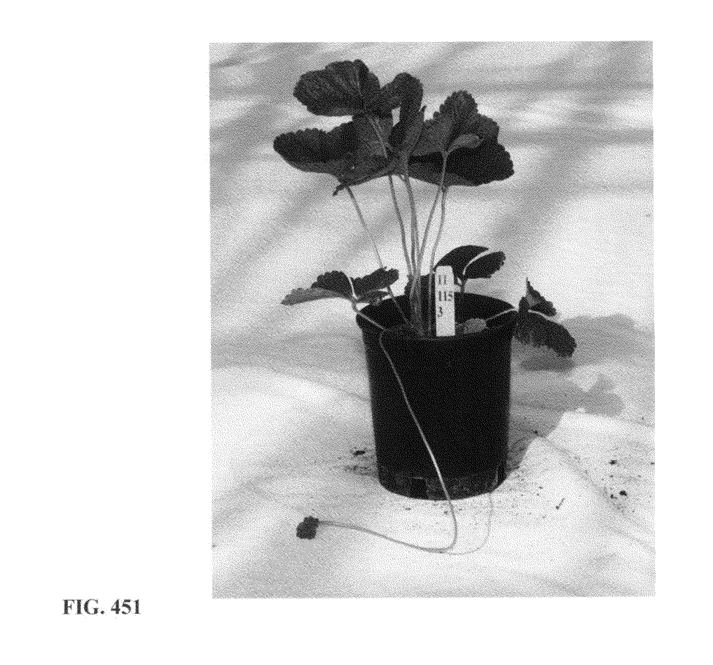

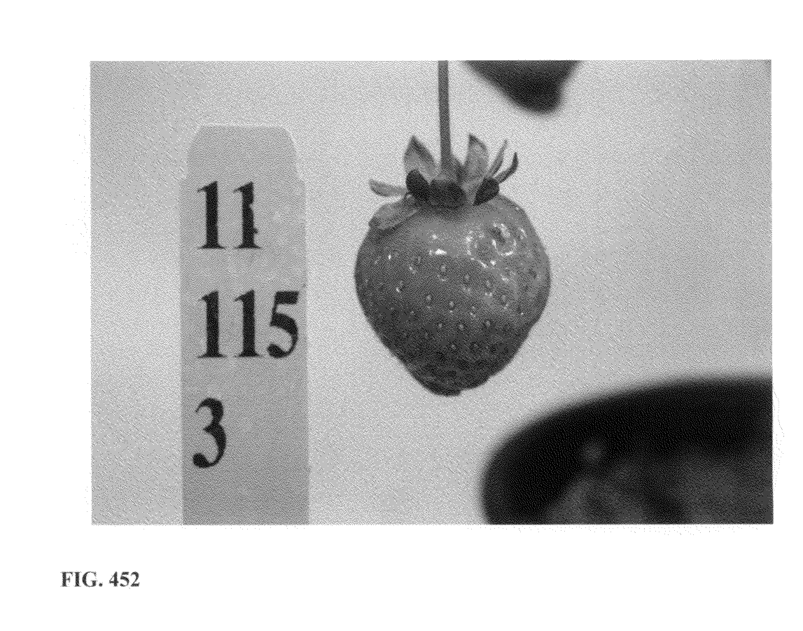

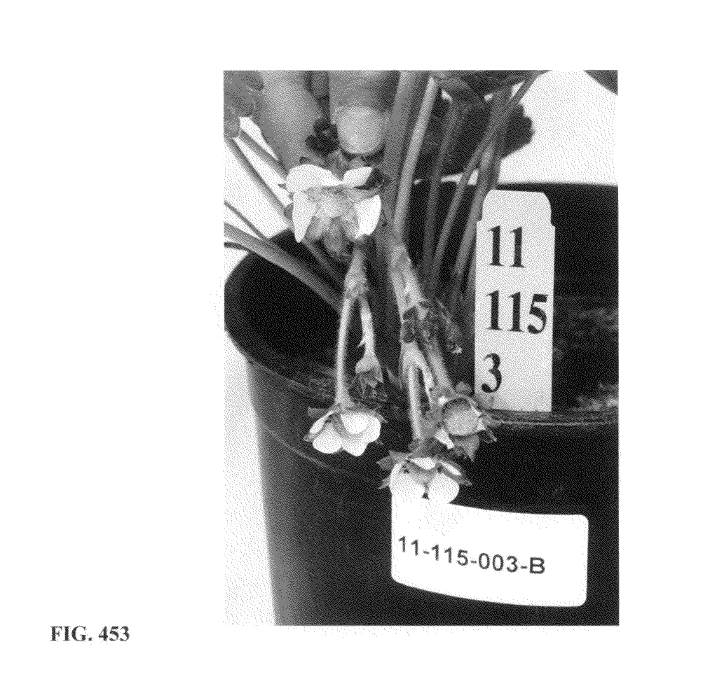

Cultivar ‘11.115-3’ has distinct disease/pathogen resistance, yield, appearance, size, and firmness. The plant of cultivar ‘11.115-3’ is characterized and distinguished in FIG. 451, the fruit of cultivar ‘11.115-3’ is characterized and distinguished in FIG. 452, and the flower of cultivar ‘11.115-3’ is characterized and distinguished in FIG. 453.

The following detailed description of cultivar ‘11.115-3’ is based on observations taken of plants and fruits (see FIGS. 451 and 452).

Certain characteristics of cultivar ‘11.115-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 151 provides data that characterizes the yield, appearance, size, and firmness of cultivar ‘11.115-3’.

TABLE 151

| Cultivar | Yield | App | Size | Firm |
|---|---|---|---|---|
| 11.115-3 | 3116 | 3.9 | 35.7 | 11.4 |

### Cultivar ‘11.116-1’

In certain aspects, the present invention relates to a new and distinctive short-day type strawberry cultivar designated as ‘11.116-1’. Cultivar ‘11.116-1’ is typical of short-day strawberry cultivars and produces fruit over an extended period when treated appropriately in arid, subtropical climates.

The strawberry cultivar ‘11.116-1’ originated from a cross between cultivar ‘Merced’ and unreleased germplasm accession ‘08.182-2’. The cultivar has been asexually reproduced by runners. The properties of this cultivar were found to be transmissible by such asexual reproduction. The cultivar is stable and reproduces true to type in successive generations of asexual reproduction.

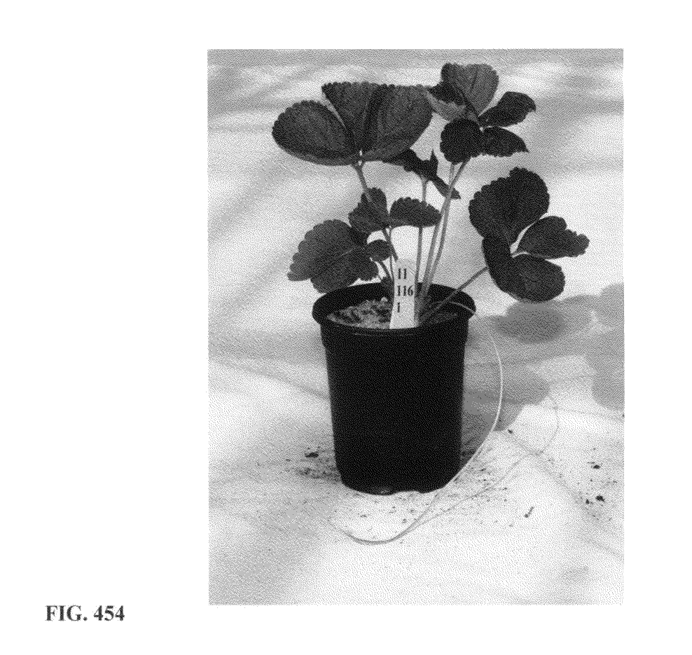

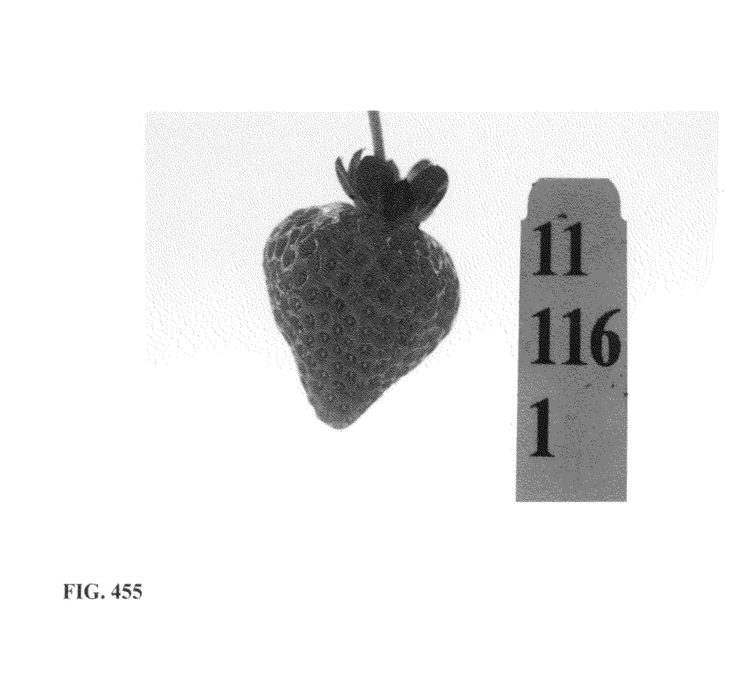

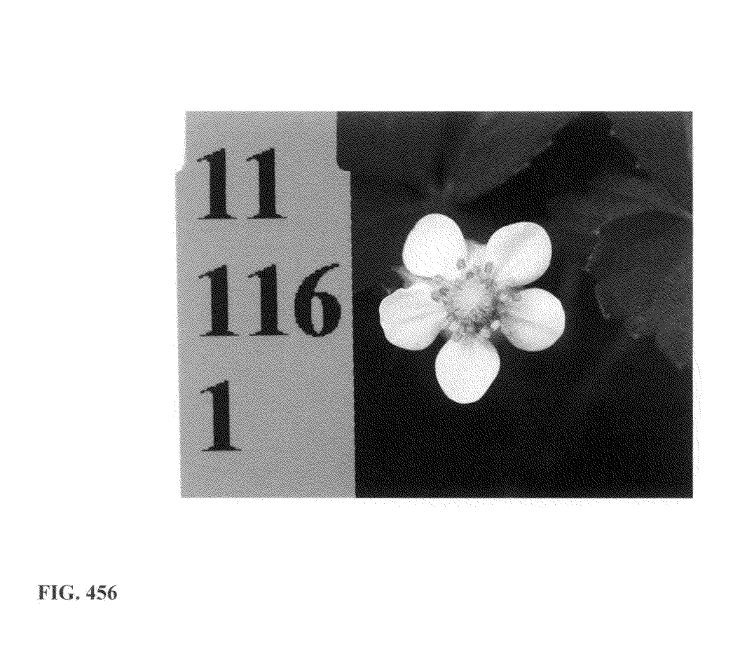

Cultivar ‘11.116-1’ has distinct disease/pathogen resistance, yield, appearance, size, and firmness. The plant of cultivar ‘11.116-1’ is characterized and distinguished in FIG. 454, the fruit of cultivar ‘11.116-1’ is characterized and distinguished in FIG. 455, and the flower of cultivar ‘11.116-1’ is characterized and distinguished in FIG. 456.

The following detailed description of cultivar ‘11.116-1’ is based on observations taken of plants and fruits (see FIGS. 454 and 455).

Certain characteristics of cultivar ‘11.116-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 152 provides data that characterizes the yield, appearance, size, and firmness of cultivar ‘11.116-1’.

TABLE 152

| Cultivar | Yield | App | Size | Firm |
|---|---|---|---|---|
| 11.116-1 | 2071 | 4.0 | 33.2 | 10.2 |

### Cultivar ‘11.129-2’

In certain aspects, the present invention relates to a new and distinctive day-neutral type strawberry cultivar designated as ‘11.129-2’. Cultivar ‘11.129-2’ is typical of day-neutral strawberry cultivars and produces fruit regardless of day length when treated appropriately in arid, subtropical climates.

The strawberry cultivar ‘11.129-2’ originated from a cross between two unreleased germplasm accessions ‘07.92-3’ and ‘07.203-1’. The cultivar has been asexually reproduced by runners. The properties of this cultivar were found to be transmissible by such asexual reproduction. The cultivar is stable and reproduces true to type in successive generations of asexual reproduction.

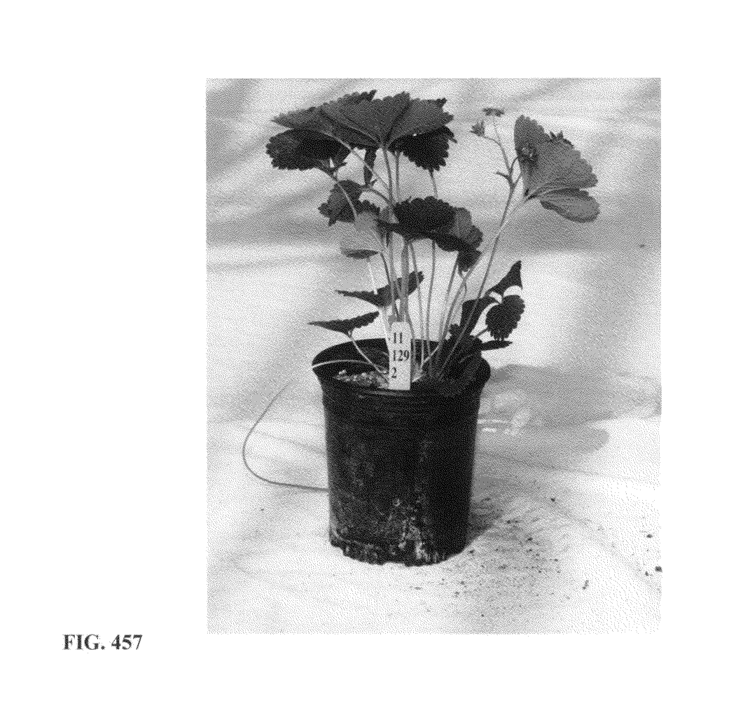

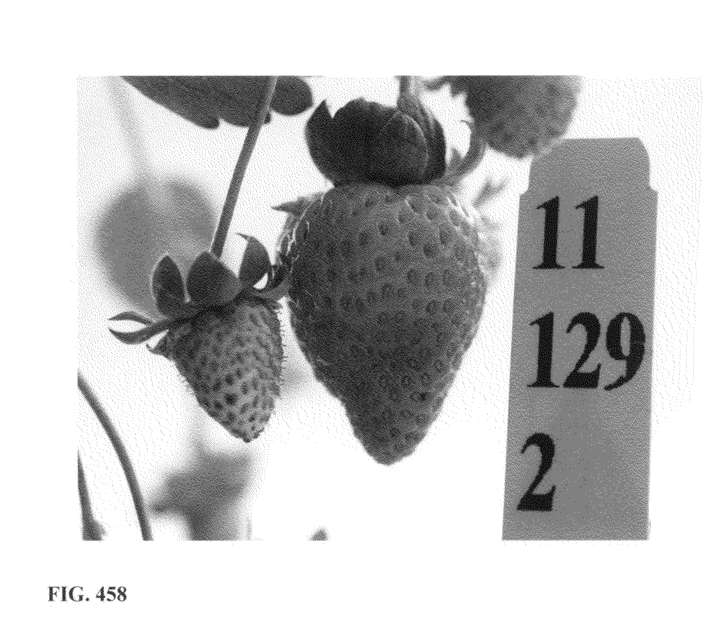

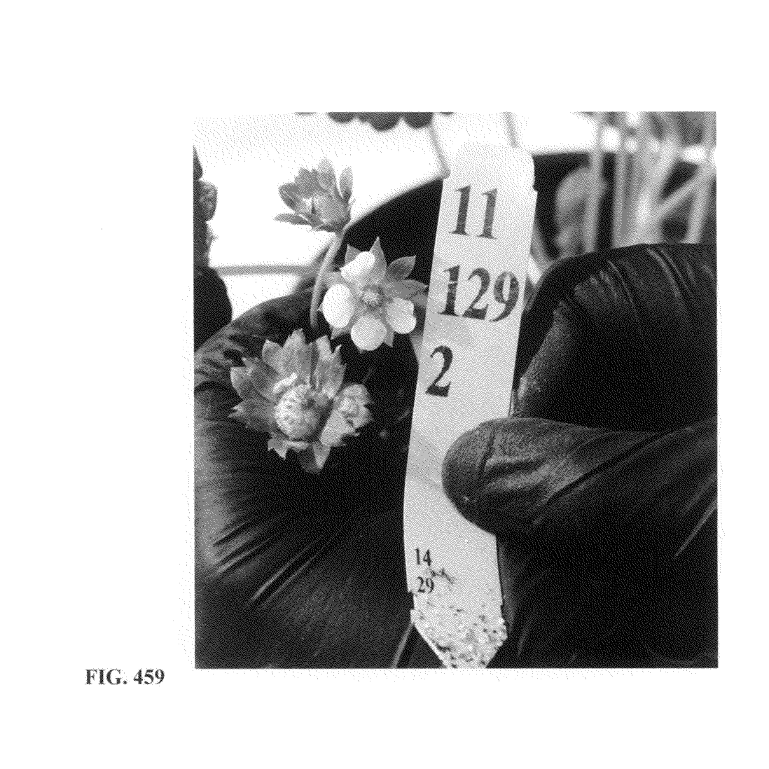

Cultivar ‘11.129-2’ has distinct disease/pathogen resistance, yield, appearance, size, and firmness. The plant of cultivar ‘11.129-2’ is characterized and distinguished in FIG. 457, the fruit of cultivar ‘11.129-2’ is characterized and distinguished in FIG. 458, and the flower of cultivar ‘11.129-2’ is characterized and distinguished in FIG. 459.

The following detailed description of cultivar ‘11.129-2’ is based on observations taken of plants and fruits (see FIGS. 457 and 458).

Certain characteristics of cultivar ‘11.129-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 153 provides data that characterizes the yield, appearance, size, and firmness of cultivar ‘11.129-2’.

TABLE 153

| Cultivar | Yield | App | Size | Firm |
|---|---|---|---|---|
| 11.129-2 | 2257 | 3.6 | 32.4 | 11.6 |

### Cultivar ‘11.141-1’

In certain aspects, the present invention relates to a new and distinctive day-neutral type strawberry cultivar designated as ‘11.141-1’. Cultivar ‘11.141-1’ is typical of day-neutral strawberry cultivars and produces fruit regardless of day length when treated appropriately in arid, subtropical climates.

The strawberry cultivar ‘11.141-1’ originated from a cross between two unreleased germplasm accessions ‘07.92-3’ and ‘08.123-1’. The cultivar has been asexually reproduced by runners. The properties of this cultivar were found to be transmissible by such asexual reproduction. The cultivar is stable and reproduces true to type in successive generations of asexual reproduction.

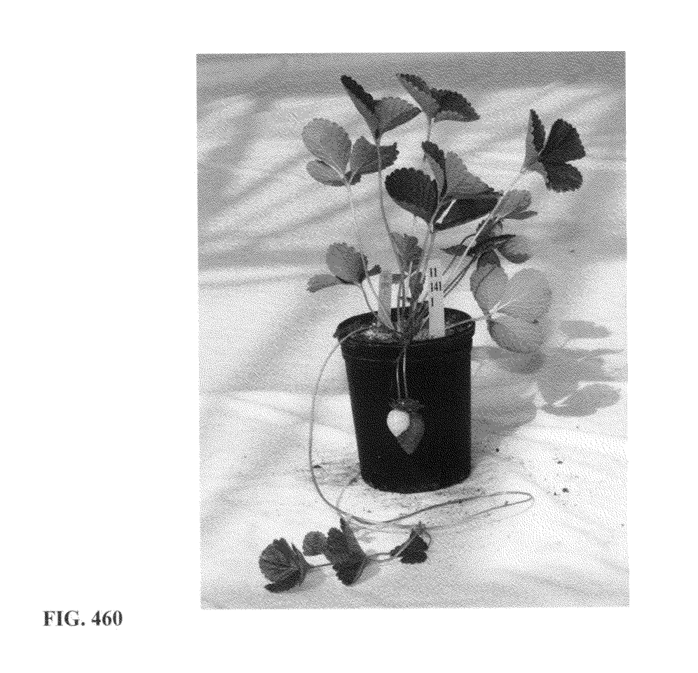

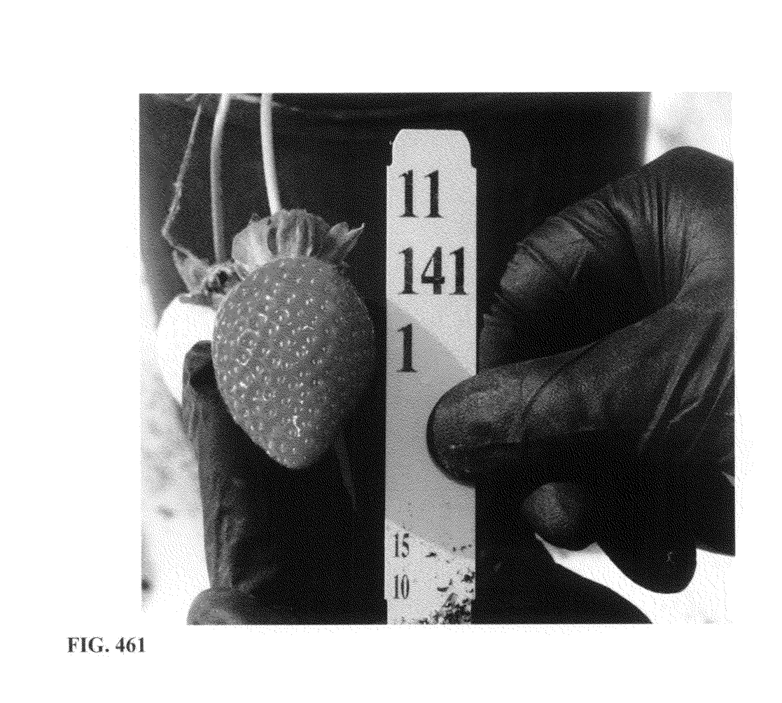

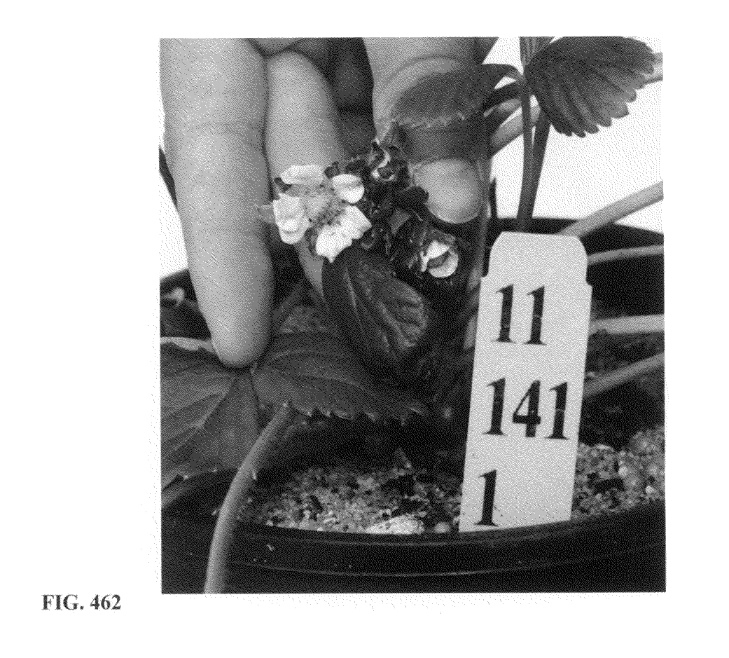

Cultivar ‘11.141-1’ has distinct disease/pathogen resistance, yield, appearance, size, and firmness. The plant of cultivar ‘11.141-1’ is characterized and distinguished in FIG. 460, the fruit of cultivar ‘11.141-1’ is characterized and distinguished in FIG. 461, and the flower of cultivar ‘11.141-1’ is characterized and distinguished in FIG. 462.

The following detailed description of cultivar ‘11.141-1’ is based on observations taken of plants and fruits (see FIGS. 460 and 461).

Certain characteristics of cultivar ‘11.141-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 154 provides data that characterizes the yield, appearance, size, and firmness of cultivar ‘11.141-1’.

TABLE 154

| Cultivar | Yield | App | Size | Firm |
|---|---|---|---|---|
| 11.141-1 | 2864 | 3.7 | 34.3 | 11.7 |

### Cultivar ‘11.145-1’

In certain aspects, the present invention relates to a new and distinctive short-day type strawberry cultivar designated as ‘11.145-1’. Cultivar ‘11.145-1’ is typical of short-day strawberry cultivars and produces fruit over an extended period when treated appropriately in arid, subtropical climates.

The strawberry cultivar ‘11.145-1’ originated from a cross between two unreleased germplasm accessions ‘06.204-1’ and ‘08.129-4’. The cultivar has been asexually reproduced by runners. The properties of this cultivar were found to be transmissible by such asexual reproduction. The cultivar is stable and reproduces true to type in successive generations of asexual reproduction.

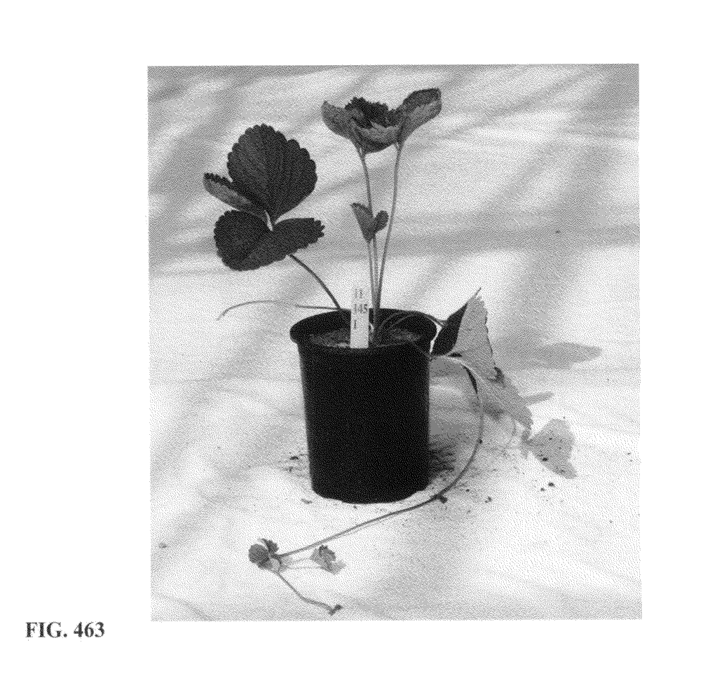

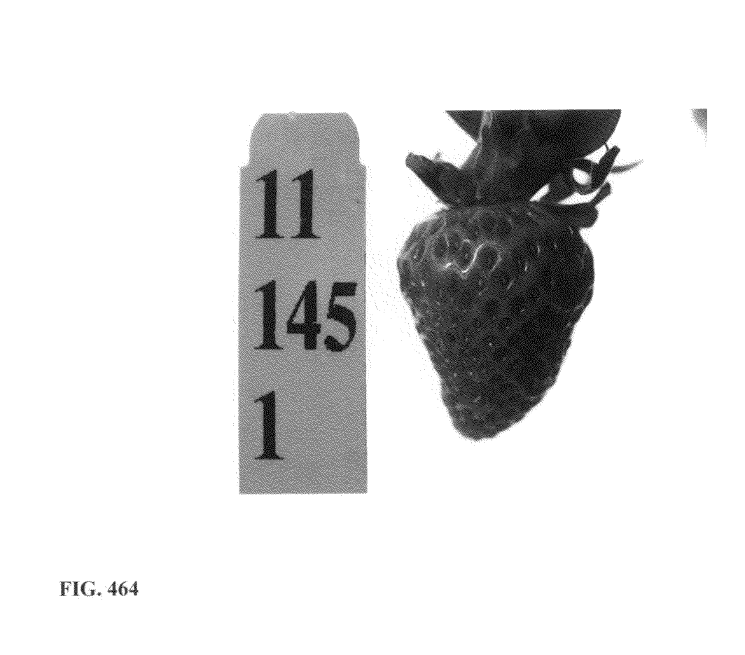

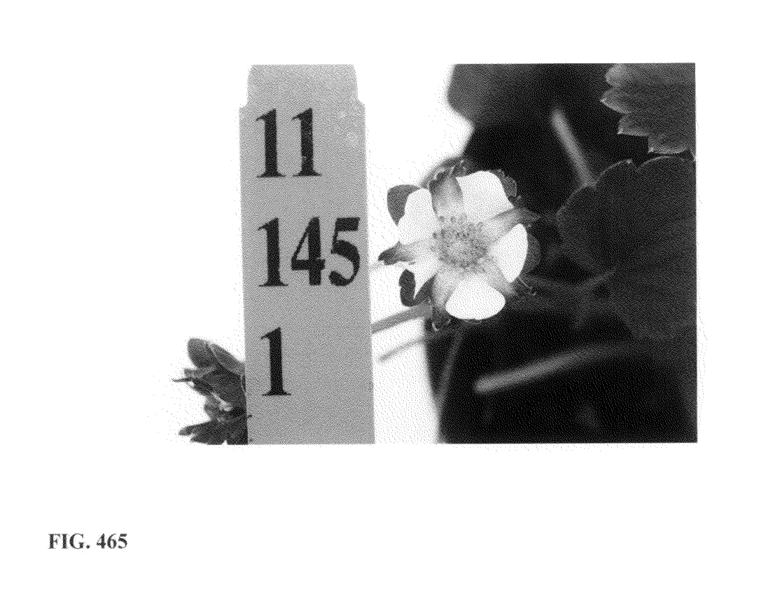

Cultivar ‘11.145-1’ has distinct disease/pathogen resistance, yield, appearance, size, and firmness. The plant of cultivar ‘11.145-1’ is characterized and distinguished in FIG. 463, the fruit of cultivar ‘11.145-1’ is characterized and distinguished in FIG. 464, and the flower of cultivar ‘11.145-1’ is characterized and distinguished in FIG. 465.

The following detailed description of cultivar ‘11.145-1’ is based on observations taken of plants and fruits (see FIGS. 463 and 464).

Certain characteristics of cultivar ‘11.145-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 155 provides data that characterizes the yield, appearance, size, and firmness of cultivar ‘11.145-1’.

TABLE 155

| Cultivar | Yield | App | Size | Firm |
|---|---|---|---|---|
| 11.145-1 | 2485 | 3.5 | 33.4 | 12.9 |

### Cultivar ‘11.151-3’

In certain aspects, the present invention relates to a new and distinctive day-neutral type strawberry cultivar designated as ‘11.151-3’. Cultivar ‘11.151-3’ is typical of day-neutral strawberry cultivars and produces fruit regardless of day length when treated appropriately in arid, subtropical climates.

The strawberry cultivar ‘11.151-3’ originated from a cross between two unreleased germplasm accessions ‘06.204-1’ and ‘08.181-1’. The cultivar has been asexually reproduced by runners. The properties of this cultivar were found to be transmissible by such asexual reproduction. The cultivar is stable and reproduces true to type in successive generations of asexual reproduction.

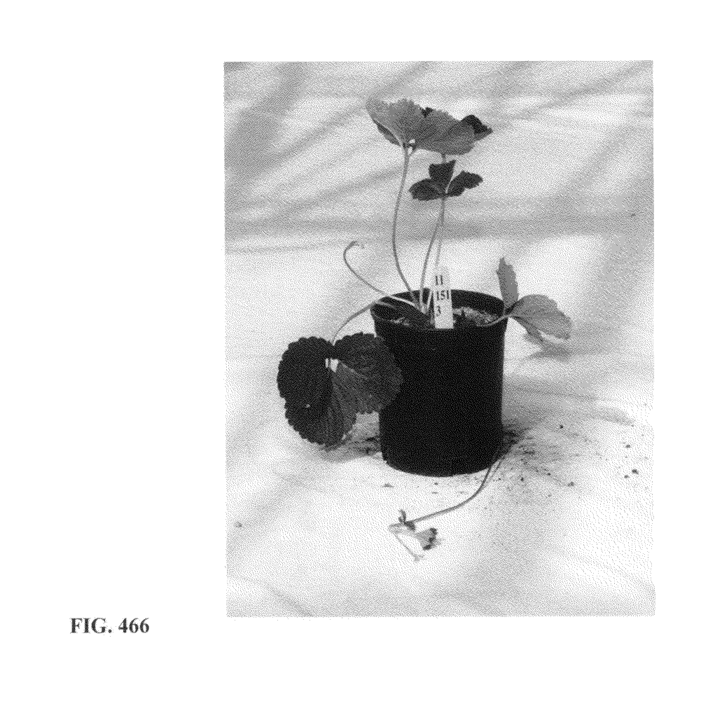

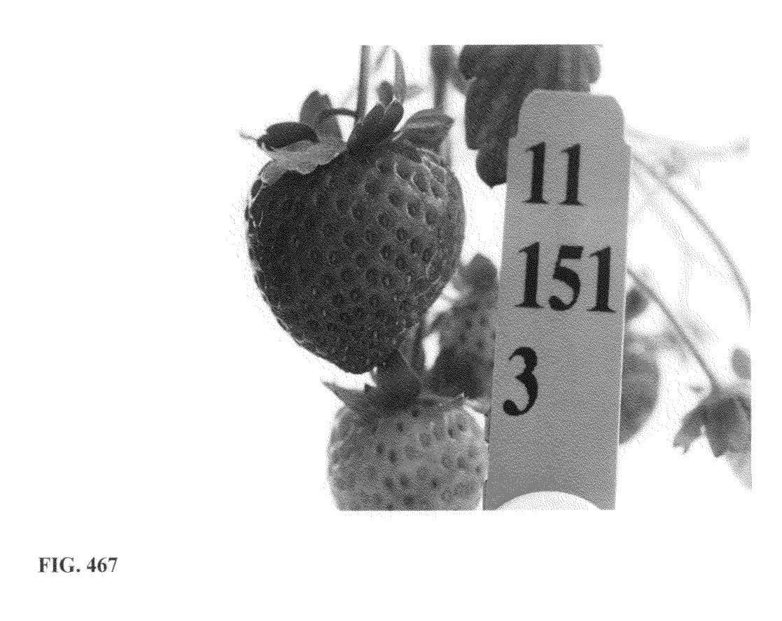

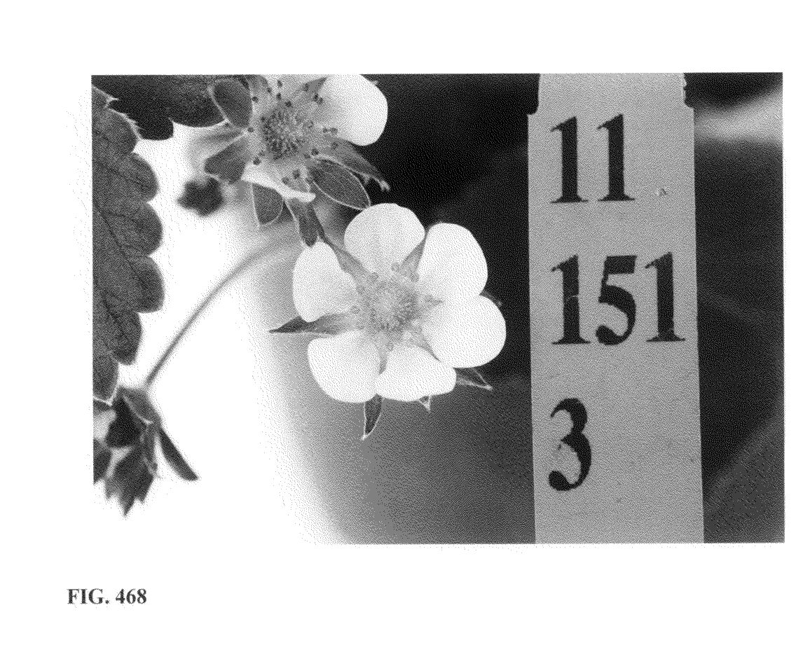

Cultivar ‘11.151-3’ has distinct disease/pathogen resistance, yield, appearance, size, and firmness. The plant of cultivar ‘11.151-3’ is characterized and distinguished in FIG. 466, the fruit of cultivar ‘11.151-3’ is characterized and distinguished in FIG. 467, and the flower of cultivar ‘11.151-3’ is characterized and distinguished in FIG. 468.

The following detailed description of cultivar ‘11.151-3’ is based on observations taken of plants and fruits (see FIGS. 466 and 467).

Certain characteristics of cultivar ‘11.151-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 156 provides data that characterizes the yield, appearance, size, and firmness of cultivar ‘11.151-3’.

TABLE 156

| Cultivar | Yield | App | Size | Firm |
|---|---|---|---|---|
| 11.151-3 | 2175 | 4.0 | 30.0 | 12.7 |

### Cultivar ‘11.151-8’

In certain aspects, the present invention relates to a new and distinctive day-neutral type strawberry cultivar designated as ‘11.151-8’. Cultivar ‘11.151-8’ is typical of day-neutral strawberry cultivars and produces fruit regardless of day length when treated appropriately in arid, subtropical climates.

The strawberry, cultivar ‘11.151-8’ originated from a cross between two unreleased germplasm accessions ‘06.204-1’ and ‘08.181-1’. The cultivar has been asexually reproduced by runners. The properties of this cultivar were found to be transmissible by such asexual reproduction. The cultivar is stable and reproduces true to type in successive generations of asexual reproduction.

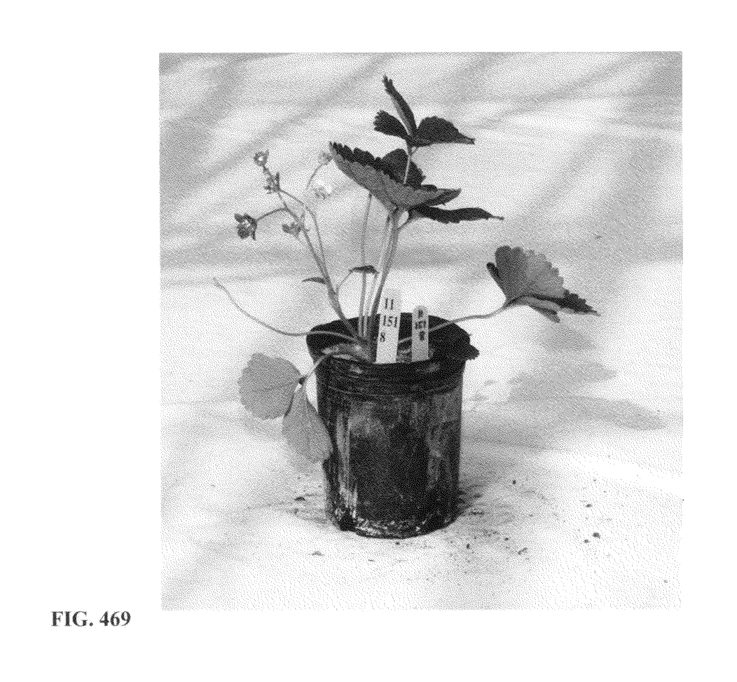

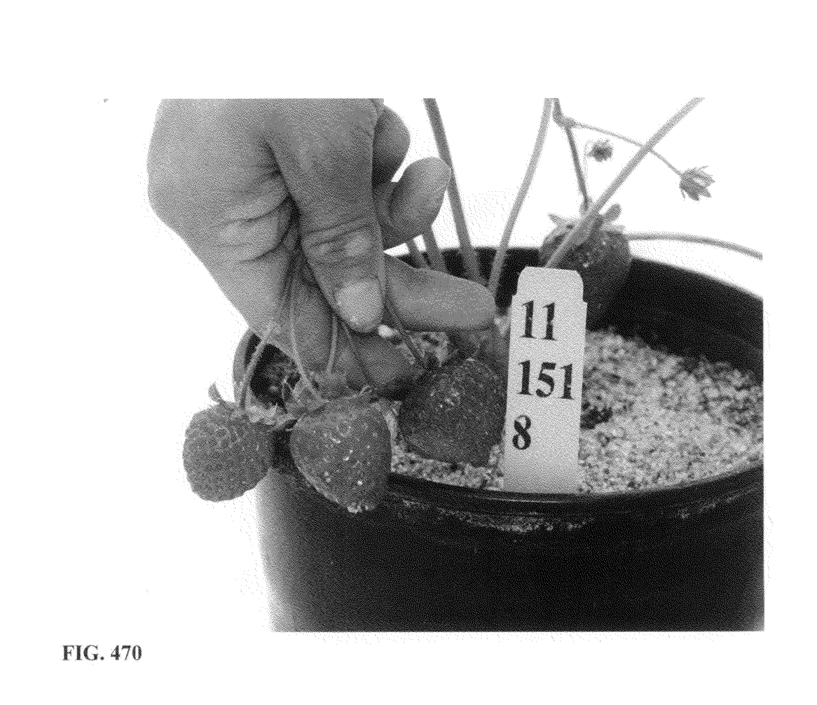

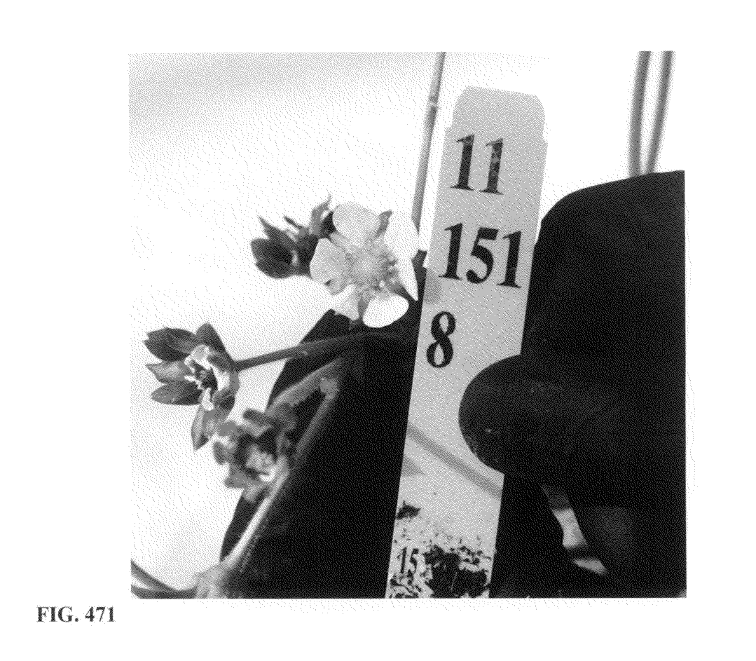

Cultivar ‘11.151-8’ has distinct disease/pathogen resistance, yield, appearance, size, and firmness. The plant of cultivar ‘11.151-8’ is characterized and distinguished in FIG. 469, the fruit of cultivar ‘11.151-8’ is characterized and distinguished in FIG. 470, and the flower of cultivar ‘11.151-8’ is characterized and distinguished in FIG. 471.

The following detailed description of cultivar ‘11.151-8’ is based on observations taken of plants and fruits (see FIGS. 469 and 470).

Certain characteristics of cultivar ‘11.151-8’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 157 provides data that characterizes the yield, appearance, size, and firmness of cultivar ‘11.151-8’.

TABLE 157

| Cultivar | Yield | App | Size | Firm |
|---|---|---|---|---|
| 11.151-8 | 2609 | 4.0 | 32.1 | 12.9 |

### Cultivar ‘11.153-2’

In certain aspects, the present invention relates to a new and distinctive short-day type strawberry cultivar designated as ‘11.153-2’. Cultivar ‘11.153-2’ is typical of short-day strawberry cultivars and produces fruit over an extended period when treated appropriately in arid, subtropical climates.

The strawberry cultivar ‘11.153-2’ originated from a cross between two unreleased germplasm accessions ‘07.92-3’ and ‘08.181-1’. The cultivar has been asexually reproduced by runners. The properties of this cultivar were found to be transmissible by such asexual reproduction. The cultivar is stable and reproduces true to type in successive generations of asexual reproduction.

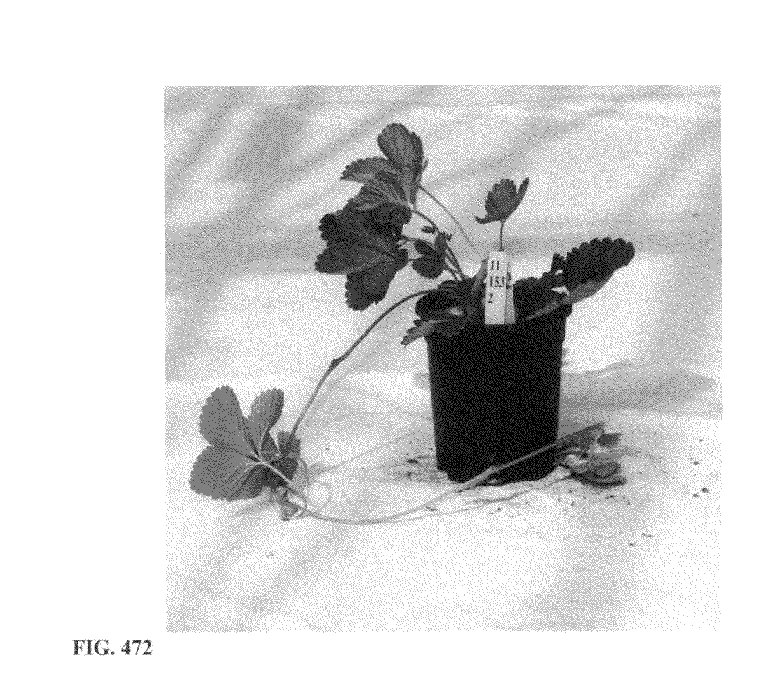

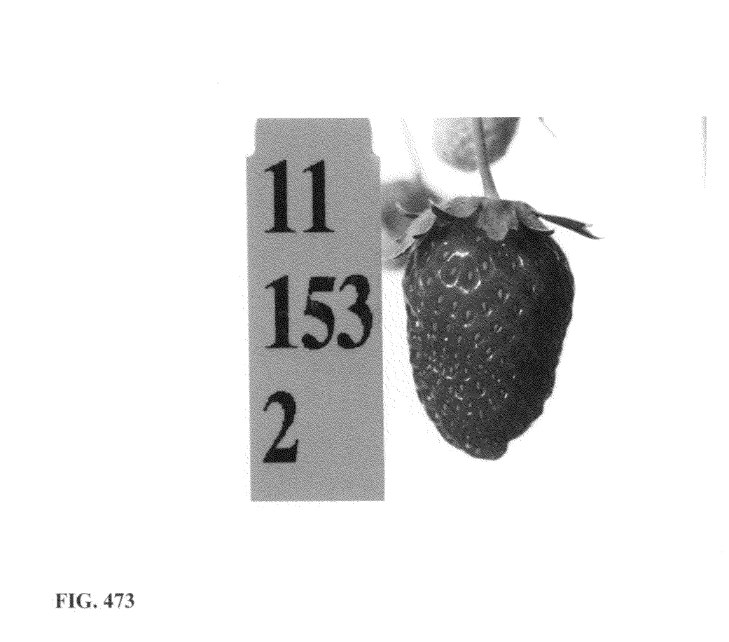

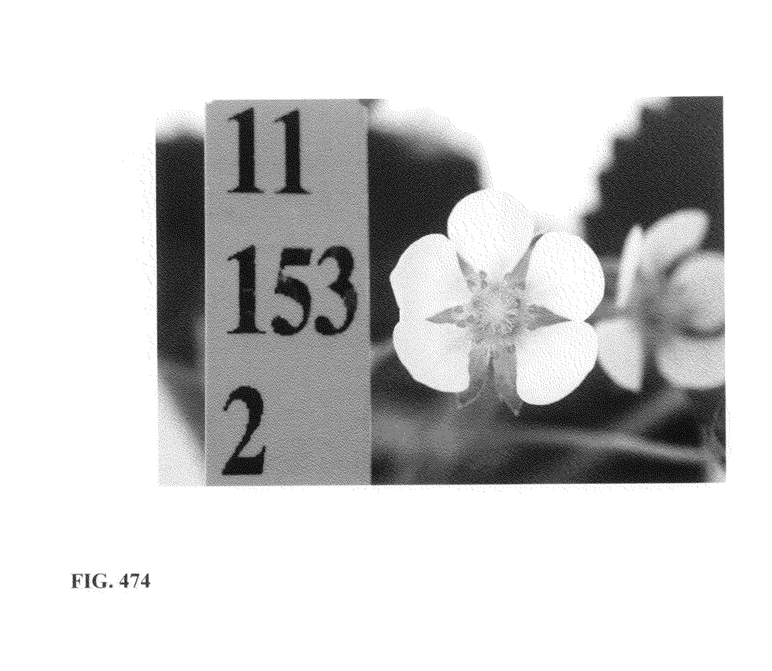

Cultivar ‘11.153-2’ has distinct disease/pathogen resistance, yield, appearance, size, and firmness. The plant of cultivar ‘11.153-2’ is characterized and distinguished in FIG. 472, the fruit of cultivar ‘11.153-2’ is characterized and distinguished in FIG. 473, and the flower of cultivar ‘11.153-2’ is characterized and distinguished in FIG. 474.

The following detailed description of cultivar ‘11.153-2’ is based on observations taken of plants and fruits (see FIGS. 472 and 473).

Certain characteristics of cultivar ‘11.153-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 158 provides data that characterizes the yield, appearance, size, and firmness of cultivar ‘11.153-2’.

TABLE 158

| Cultivar | Yield | App | Size | Firm |
|---|---|---|---|---|
| 11.153-2 | 2200 | 3.7 | 30.7 | 10.6 |

### Cultivar ‘11.153-3’

In certain aspects, the present invention relates to a new and distinctive day-neutral type strawberry cultivar designated as ‘11.153-3’. Cultivar ‘11.153-3’ is typical of day-neutral strawberry cultivars and produces fruit regardless of day length when treated appropriately in arid, subtropical climates.

The strawberry cultivar ‘11.153-3’ originated from a cross between two unreleased germplasm accessions ‘07.92-3’ and ‘08.181-1’. The cultivar has been asexually reproduced by runners. The properties of this cultivar were found to be transmissible by such asexual reproduction. The cultivar is stable and reproduces true to type in successive generations of asexual reproduction.

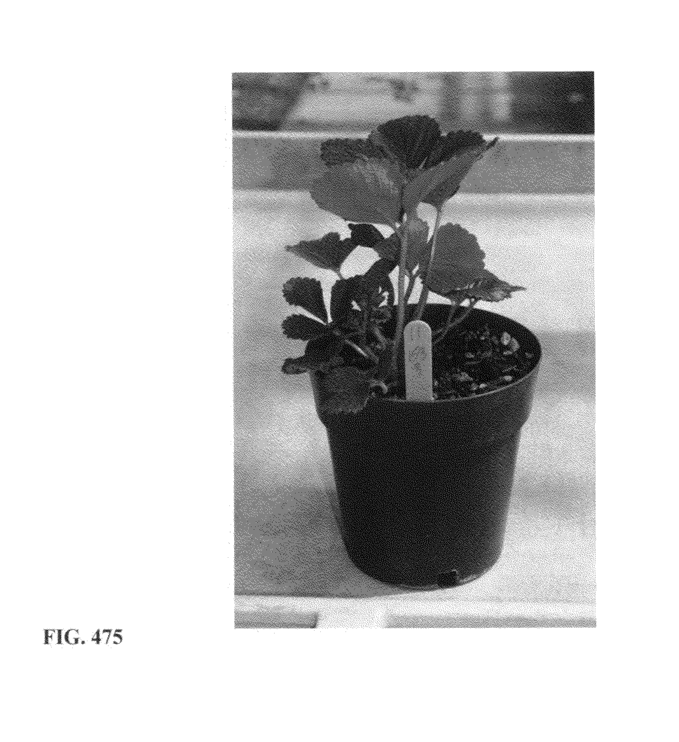

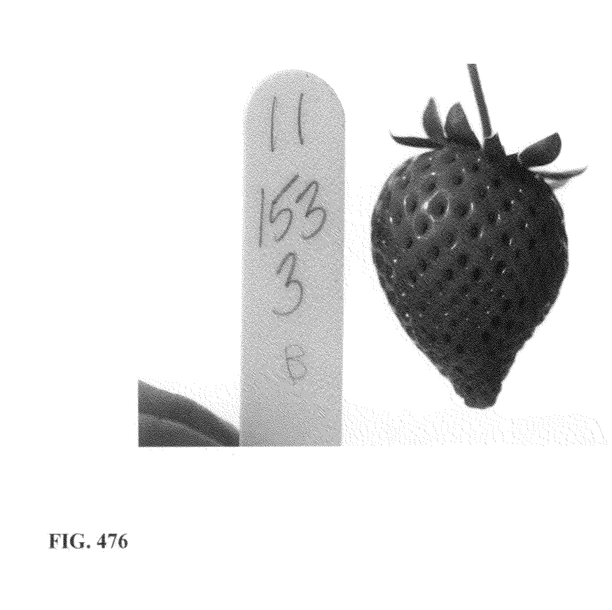

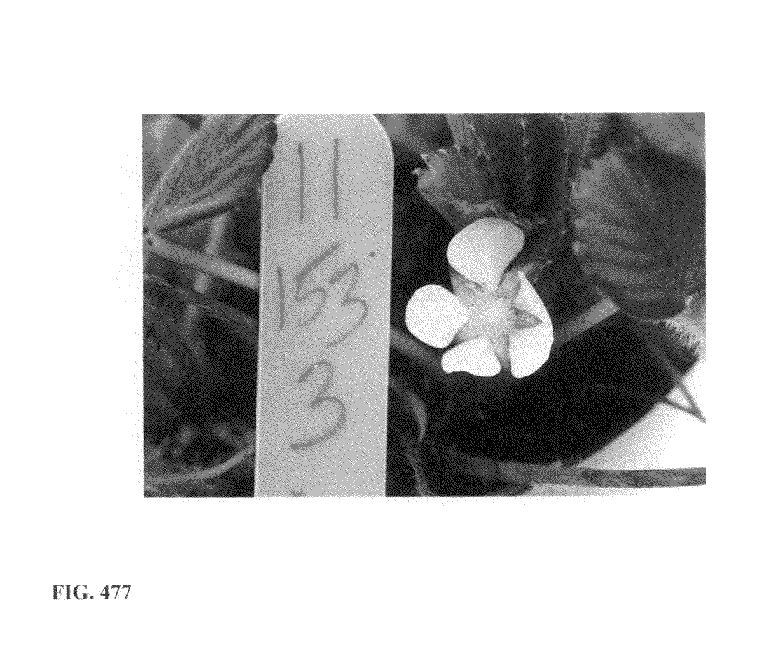

Cultivar ‘11.153-3’ has distinct disease/pathogen resistance, yield, appearance, size, and firmness. The plant of cultivar ‘11.153-3’ is characterized and distinguished in FIG. 475, the fruit of cultivar ‘11.153-3’ is characterized and distinguished in FIG. 476, and the flower of cultivar ‘11.153-3’ is characterized and distinguished in FIG. 477.

The following detailed description of cultivar ‘11.153-3’ is based on observations taken of plants and fruits (see FIGS. 475 and 476).

Certain characteristics of cultivar ‘11.153-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 159 provides data that characterizes the yield, appearance, size, and firmness of cultivar ‘11.153-3’.

TABLE 159

| Cultivar | Yield | App | Size | Firm |
|---|---|---|---|---|
| 11.153-3 | 2707 | 3.9 | 31.4 | 13.7 |

### Cultivar ‘11.154-5’

In certain aspects, the present invention relates to a new and distinctive day-neutral type strawberry cultivar designated as ‘11.154-5’. Cultivar ‘11.154-5’ is typical of day-neutral strawberry cultivars and produces fruit regardless of day length when treated appropriately in arid, subtropical climates.

The strawberry cultivar ‘11.154-5’ originated from a cross between two unreleased germplasm accessions ‘08.75-1’ and ‘08.181-1’. The cultivar has been asexually reproduced by runners. The properties of this cultivar were found to be transmissible by such asexual reproduction. The cultivar is stable and reproduces true to type in successive generations of asexual reproduction.

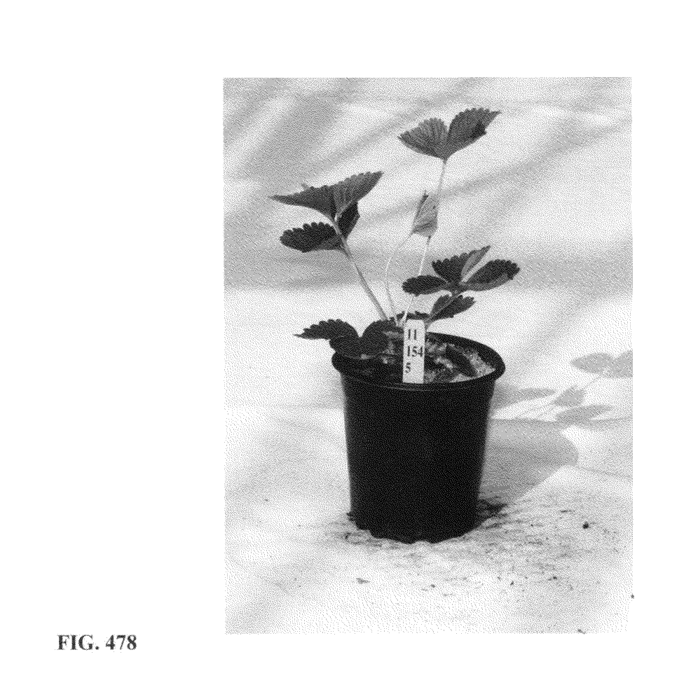

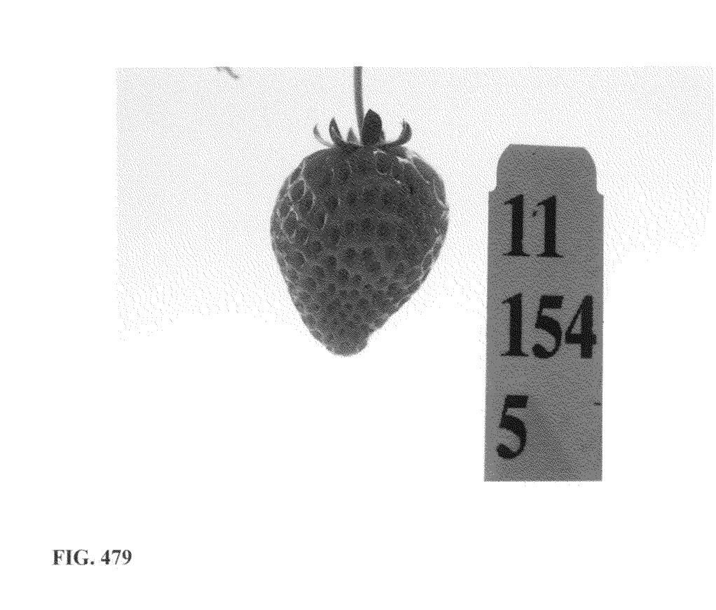

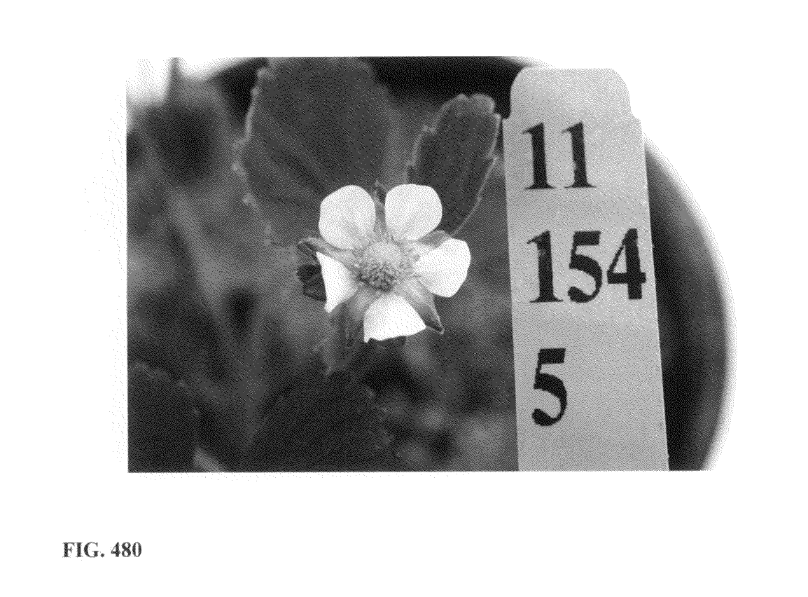

Cultivar ‘11.154-5’ has distinct disease/pathogen resistance, yield, appearance, size, and firmness. The plant of cultivar ‘11.154-5’ is characterized and distinguished in FIG. 478, the fruit of cultivar ‘11.154-5’ is characterized and distinguished in FIG. 479, and the flower of cultivar ‘11.154-5’ is characterized and distinguished in FIG. 480.

The following detailed description of cultivar ‘11.154-5’ is based on observations taken of plants and fruits (see FIGS. 478 and 479).

Certain characteristics of cultivar ‘11.154-5’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 160 provides data that characterizes the yield, appearance, size, and firmness of cultivar ‘11.154-5’.

TABLE 160

| Cultivar | Yield | App | Size | Firm |
|---|---|---|---|---|
| 11.154-5 | 2729 | 3.8 | 30.3 | 11.9 |

### Cultivar ‘11.157-1’

In certain aspects, the present invention relates to a new and distinctive day-neutral type strawberry cultivar designated as ‘11.157-1’. Cultivar ‘11.157-1’ is typical of day-neutral strawberry cultivars and produces fruit regardless of day length when treated appropriately in arid, subtropical climates.

The strawberry cultivar ‘11.157-1’ originated from a cross between two unreleased germplasm accessions ‘07.120-3’ and ‘05.132-1’. The cultivar has been asexually reproduced by runners. The properties of this cultivar were found to be transmissible by such asexual reproduction. The cultivar is stable and reproduces true to type in successive generations of asexual reproduction.

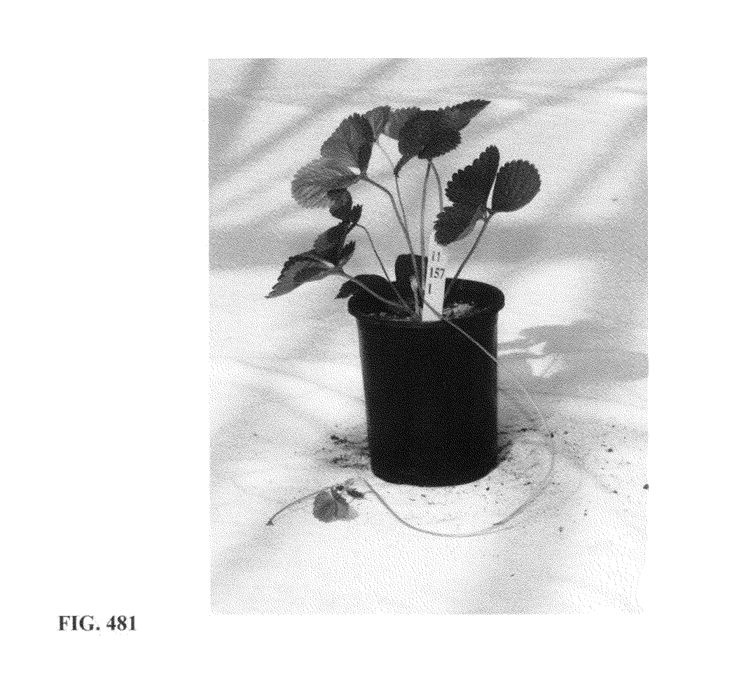

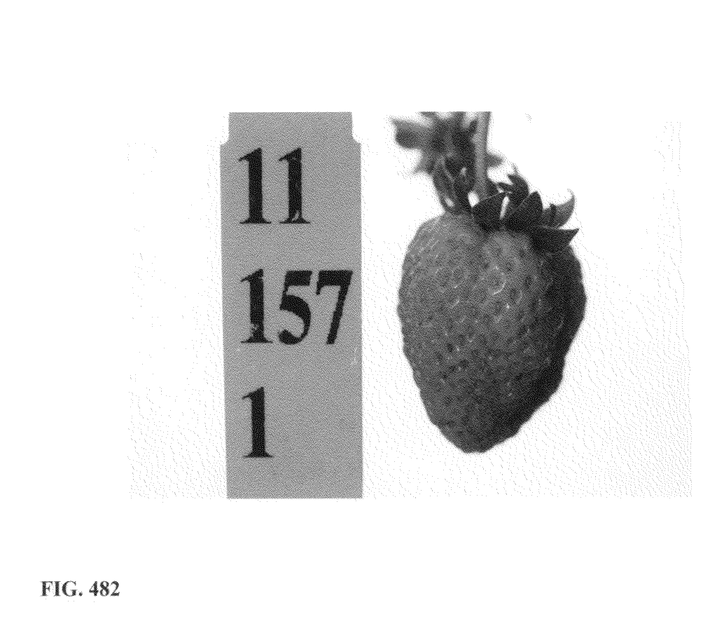

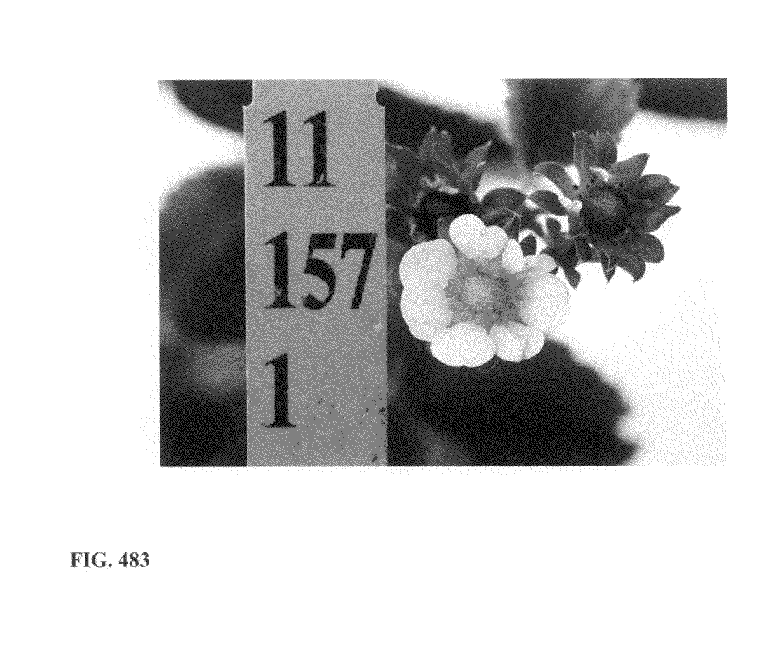

Cultivar ‘11.157-1’ has distinct disease/pathogen resistance, yield, appearance, size, and firmness. The plant of cultivar ‘11.157-1’ is characterized and distinguished in FIG. 481, the fruit of cultivar ‘11.157-1’ is characterized and distinguished in FIG. 482, and the flower of cultivar ‘11.157-1’ is characterized and distinguished in FIG. 483.

The following detailed description of cultivar ‘11.157-1’ is based on observations taken of plants and fruits (see FIGS. 481 and 482).

Certain characteristics of cultivar ‘11.157-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 161 provides data that characterizes the yield, appearance, size, and firmness of cultivar ‘11.157-1’.

TABLE 161

| Cultivar | Yield | App | Size | Firm |
|---|---|---|---|---|
| 11.157-1 | 1514 | 3.6 | 30.6 | 12.0 |

### Cultivar ‘11.158-1’

In certain aspects, the present invention relates to a new and distinctive day-neutral type strawberry cultivar designated as ‘11.158-1’. Cultivar ‘11.158-1’ is typical of day-neutral strawberry cultivars and produces fruit regardless of day length when treated appropriately in arid, subtropical climates.

The strawberry cultivar ‘11.158-1’ originated from a cross between cultivar ‘Merced’ and unreleased germplasm accession ‘05.132-1’. The cultivar has been asexually reproduced by runners. The properties of this cultivar were found to be transmissible by such asexual reproduction. The cultivar is stable and reproduces true to type in successive generations of asexual reproduction.

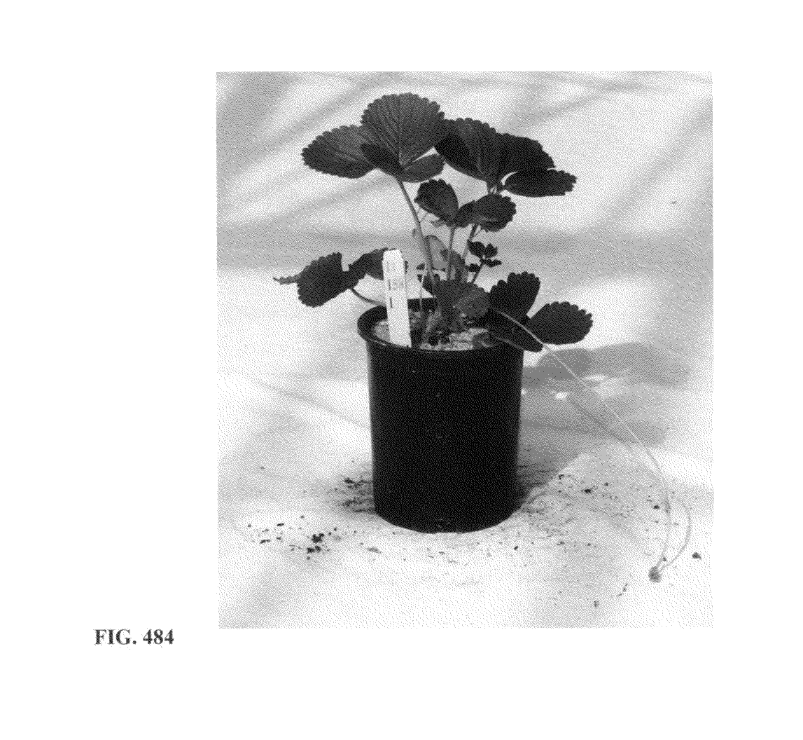

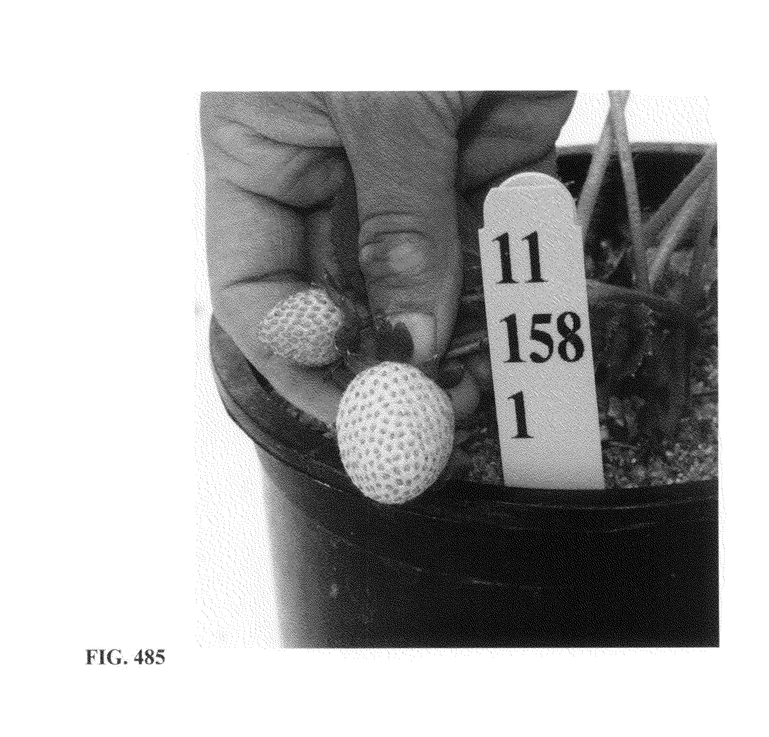

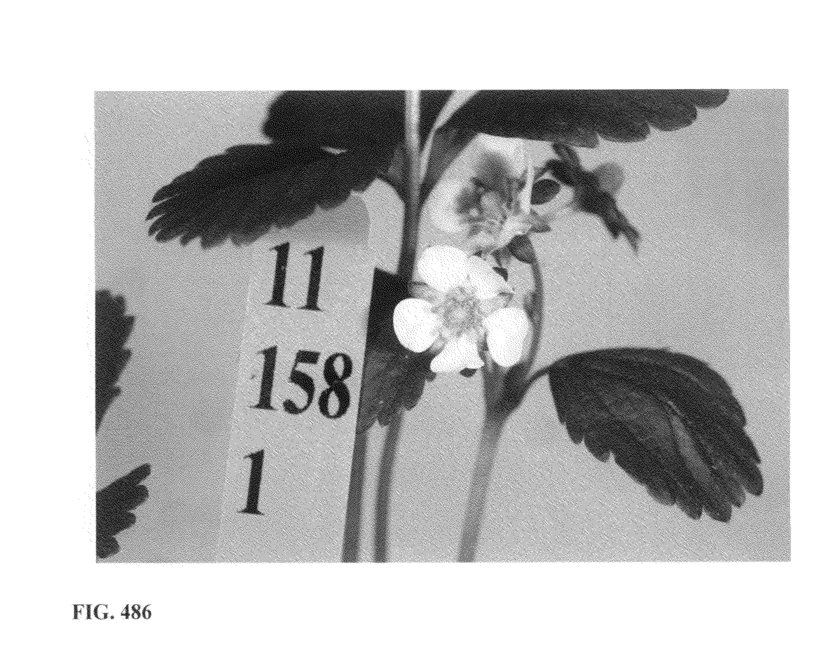

Cultivar ‘11.158-1’ has distinct disease/pathogen resistance, yield, appearance, size, and firmness. The plant of cultivar ‘11.158-1’ is characterized and distinguished in FIG. 484, the fruit of cultivar ‘11.158-1’ is characterized and distinguished in FIG. 485, and the flower of cultivar ‘11.158-1’ is characterized and distinguished in FIG. 486.

The following detailed description of cultivar ‘11.158-1’ is based on observations taken of plants and fruits (see FIGS. 484 and 485).

Certain characteristics of cultivar ‘11.158-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 162 provides data that characterizes the yield, appearance, size, and firmness of cultivar ‘11.158-1’.

TABLE 162

| Cultivar | Yield | App | Size | Firm |
|---|---|---|---|---|
| 11.158-1 | 1735 | 3.8 | 30.1 | 12.3 |

### Cultivar ‘11.167-4’

In certain aspects, the present invention relates to a new and distinctive day-neutral type strawberry cultivar designated as ‘11.167-4’. Cultivar ‘11.167-4’ is typical of day-neutral strawberry cultivars and produces fruit regardless of day length when treated appropriately in arid, subtropical climates.

The strawberry cultivar ‘11.167-4’ originated from a cross between two unreleased germplasm accessions ‘07.43-1’ and ‘06.110-4’. The cultivar has been asexually reproduced by runners. The properties of this cultivar were found to be transmissible by such asexual reproduction. The cultivar is stable and reproduces true to type in successive generations of asexual reproduction.

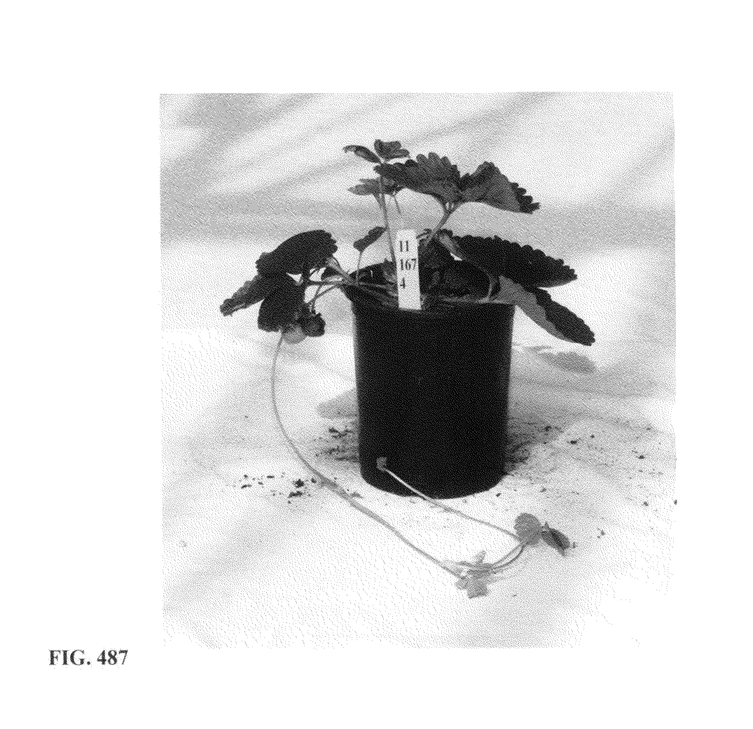

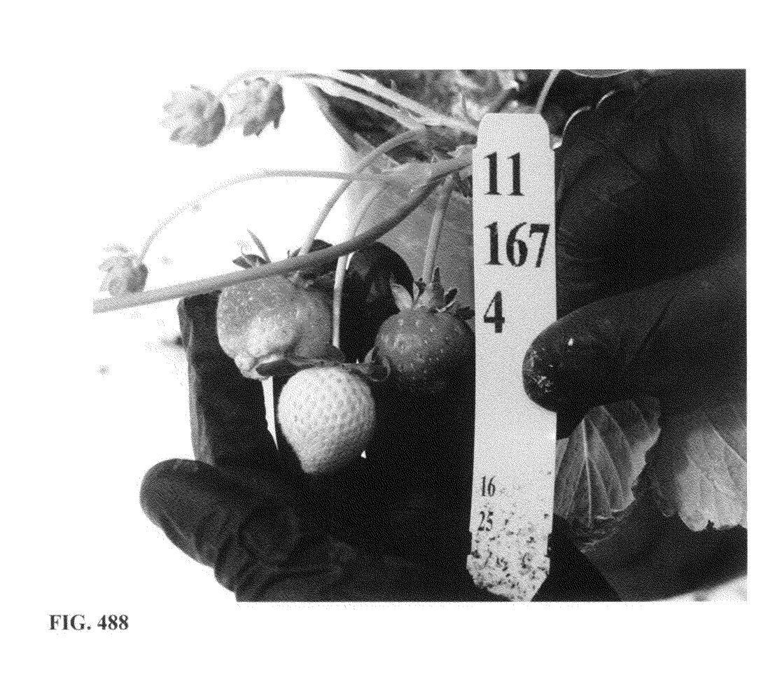

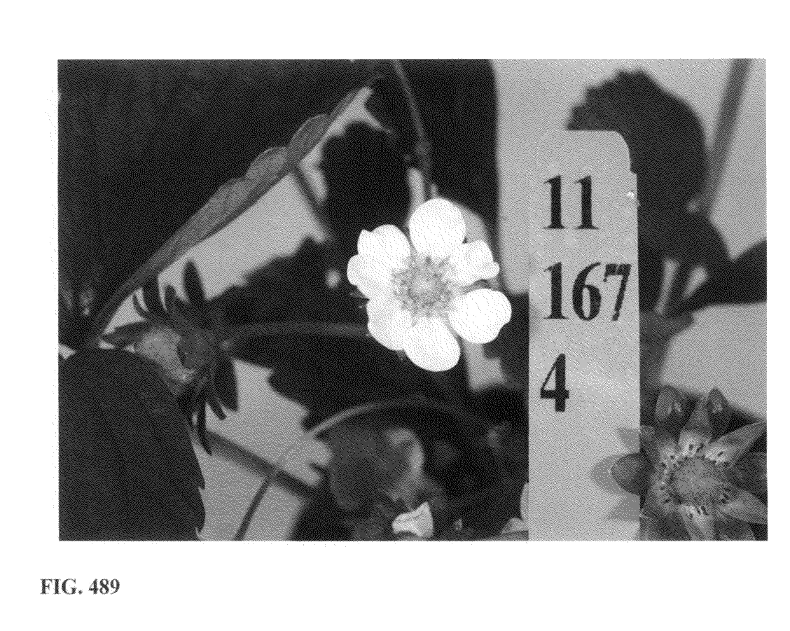

Cultivar ‘11.167-4’ has distinct disease/pathogen resistance, yield, appearance, size, and firmness. The plant of cultivar ‘11.167-4’ is characterized and distinguished in FIG. 487, the fruit of cultivar ‘11.167-4’ is characterized and distinguished in FIG. 488, and the flower of cultivar ‘11.167-4’ is characterized and distinguished in FIG. 489.

The following detailed description of cultivar ‘11.167-4’ is based on observations taken of plants and fruits (see FIGS. 487 and 488).

Certain characteristics of cultivar ‘11.167-4’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 163 provides data that characterizes the yield, appearance, size, and firmness of cultivar ‘11.167-4’.

TABLE 163

| Cultivar | Yield | App | Size | Firm |
|---|---|---|---|---|
| 11.167-4 | 2762 | 3.6 | 32.4 | 13.3 |

### Cultivar ‘11.179-3’

In certain aspects, the present invention relates to a new and distinctive short-day type strawberry cultivar designated as ‘11.179-3’. Cultivar ‘11.179-3’ is typical of short-day strawberry cultivars and produces fruit over an extended period when treated appropriately in arid, subtropical climates.

The strawberry cultivar ‘11.179-3’ originated from a cross between two unreleased germplasm accessions ‘07.43-1’ and ‘07.148-3’. The cultivar has been asexually reproduced by runners. The properties of this cultivar were found to be transmissible by such asexual reproduction. The cultivar is stable and reproduces true to type in successive generations of asexual reproduction.

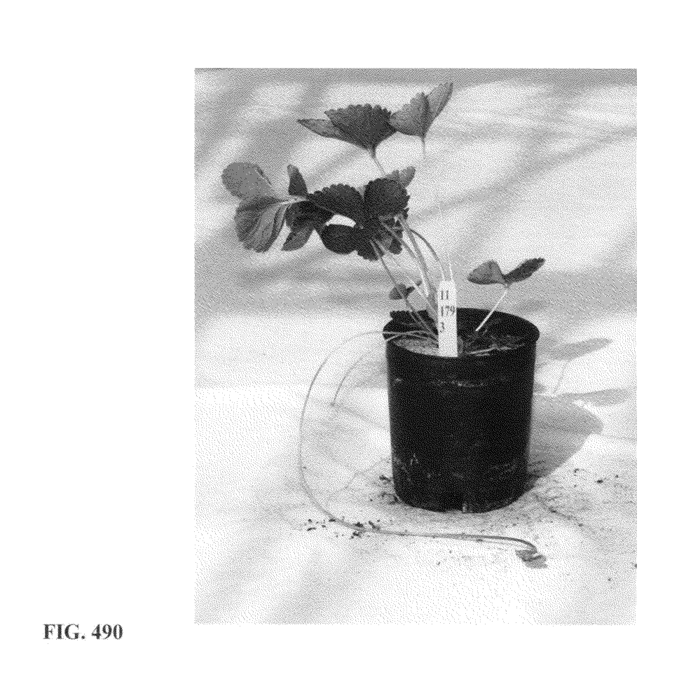

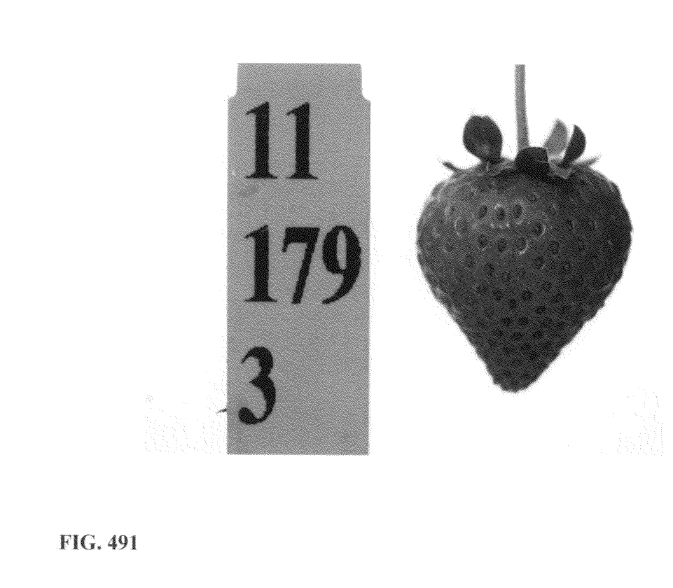

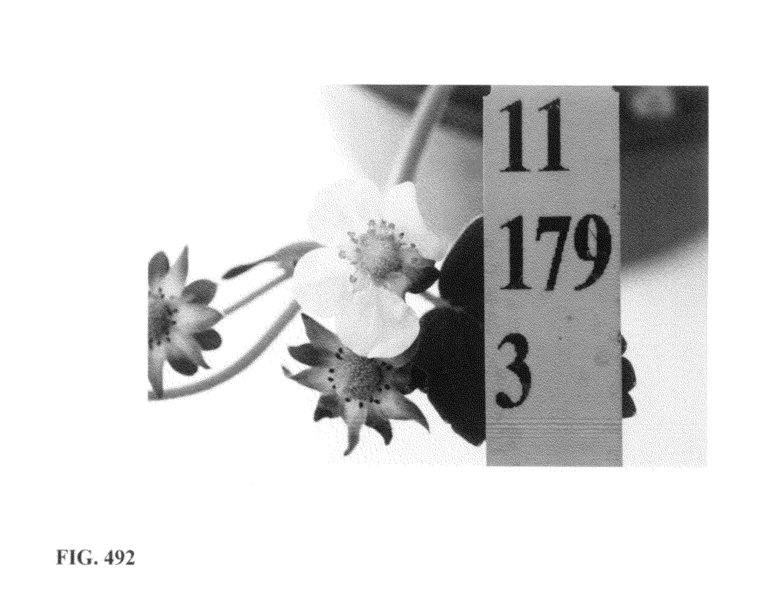

Cultivar ‘11.179-3’ has distinct disease/pathogen resistance, yield, appearance, size, and firmness. The plant of cultivar ‘11.179-3’ is characterized and distinguished in FIG. 490, the fruit of cultivar ‘11.179-3’ is characterized and distinguished in FIG. 491, and the flower of cultivar ‘11.179-3’ is characterized and distinguished in FIG. 492.

The following detailed description of cultivar ‘11.179-3’ is based on observations taken of plants and fruits (see FIGS. 490 and 491).

Certain characteristics of cultivar ‘11.179-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 164 provides data that characterizes the yield, appearance, size, and firmness of cultivar ‘11.179-3’.

TABLE 164

| Cultivar | Yield | App | Size | Firm |
|---|---|---|---|---|
| 11.179-3 | 2367 | 3.4 | 28.8 | 11.7 |

### Cultivar ‘11.180-1’

In certain aspects, the present invention relates to a new and distinctive day-neutral type strawberry cultivar designated as ‘11.180-1’. Cultivar ‘11.180-1’ is typical of day-neutral strawberry cultivars and produces fruit regardless of day length when treated appropriately in arid, subtropical climates.

The strawberry cultivar ‘11.180-1’ originated from a cross between two unreleased germplasm accessions ‘07.64-2’ and ‘07.148-3’. The cultivar has been asexually reproduced by runners. The properties of this cultivar were found to be transmissible by such asexual reproduction. The cultivar is stable and reproduces true to type in successive generations of asexual reproduction.

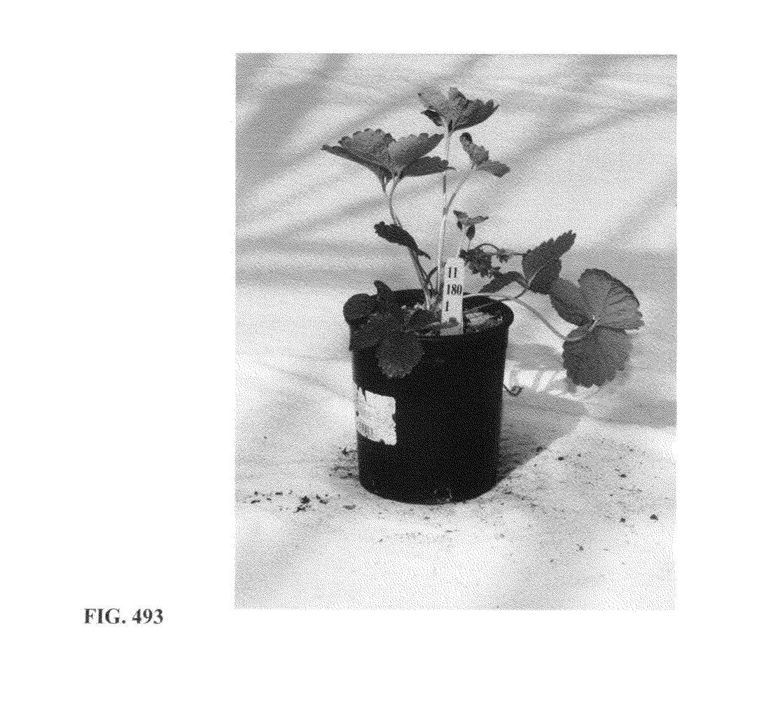

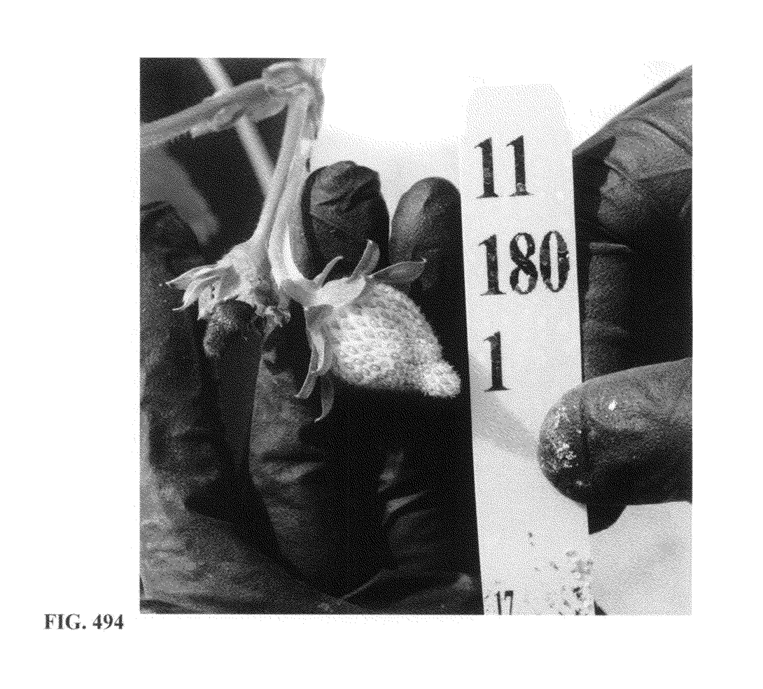

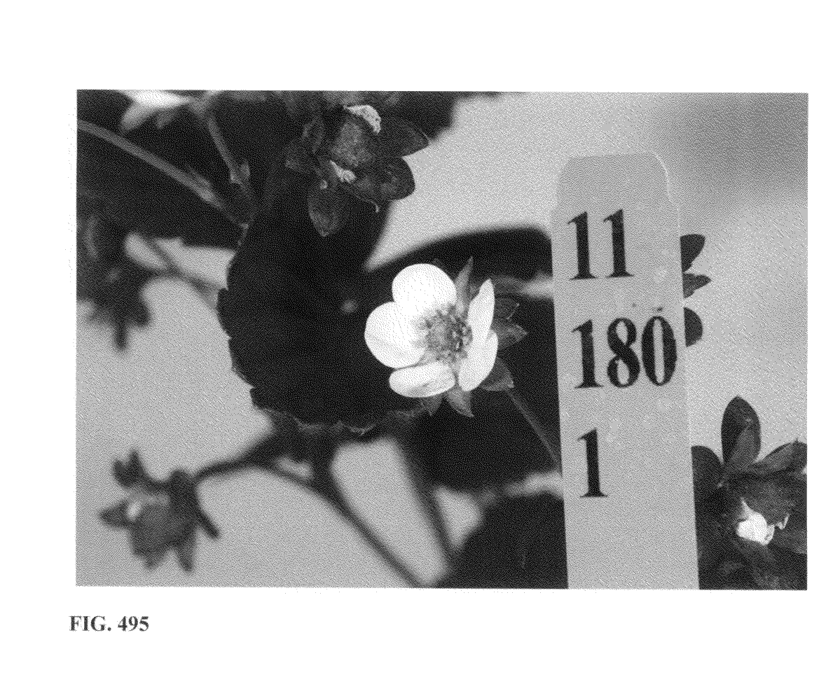

Cultivar ‘11.180-1’ has distinct disease/pathogen resistance, yield, appearance, size, and firmness. The plant of cultivar ‘11.180-1’ is characterized and distinguished in FIG. 493, the fruit of cultivar ‘11.180-1’ is characterized and distinguished in FIG. 494, and the flower of cultivar ‘11.180-1’ is characterized and distinguished in FIG. 495.

The following detailed description of cultivar ‘11.180-1’ is based on observations taken of plants and fruits (see FIGS. 493 and 494).

Certain characteristics of cultivar ‘11.180-1’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 165 provides data that characterizes the yield, appearance, size, and firmness of cultivar ‘11.180-1’.

TABLE 165

| Cultivar | Yield | App | Size | Firm |
|---|---|---|---|---|
| 11.180-1 | 2786 | 4.0 | 34.1 | 10.2 |

### Cultivar ‘11.181-2’

In certain aspects, the present invention relates to a new and distinctive day-neutral type strawberry cultivar designated as ‘11.181-2’. Cultivar ‘11.181-2’ is typical of day-neutral strawberry cultivars and produces fruit regardless of day length when treated appropriately in arid, subtropical climates.

The strawberry cultivar ‘11.181-2’ originated from a cross between two unreleased germplasm accessions ‘07.120-3’ and ‘07.148-3’. The cultivar has been asexually reproduced by runners. The properties of this cultivar were found to be transmissible by such asexual reproduction. The cultivar is stable and reproduces true to type in successive generations of asexual reproduction.

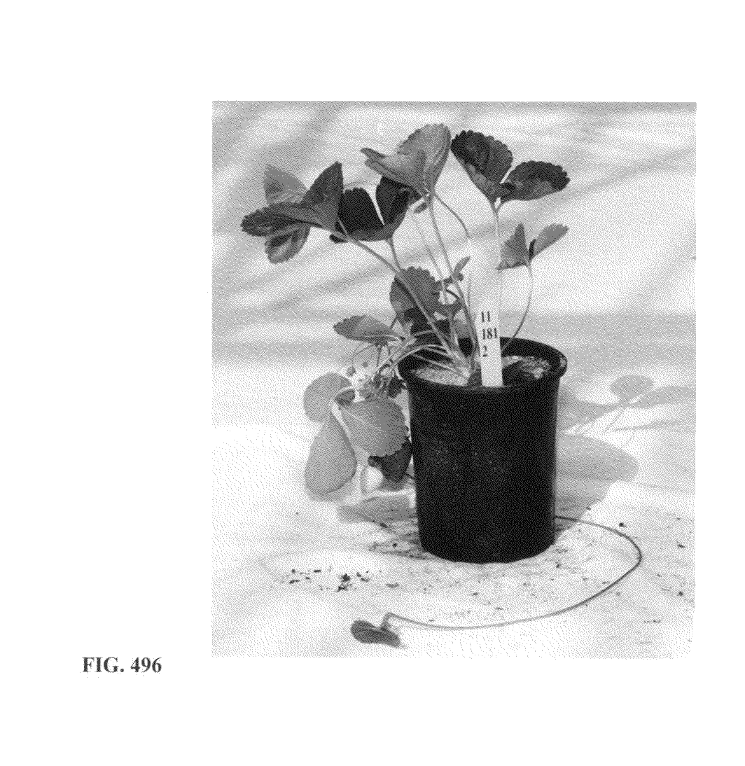

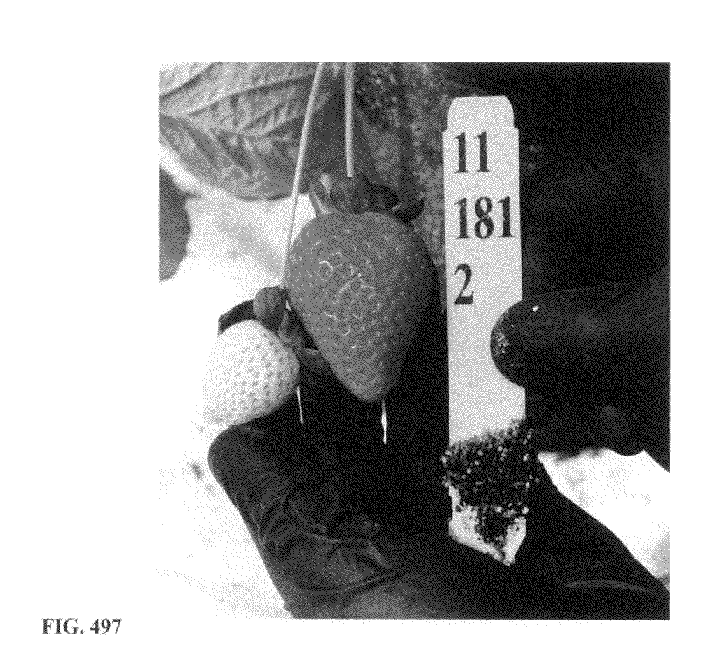

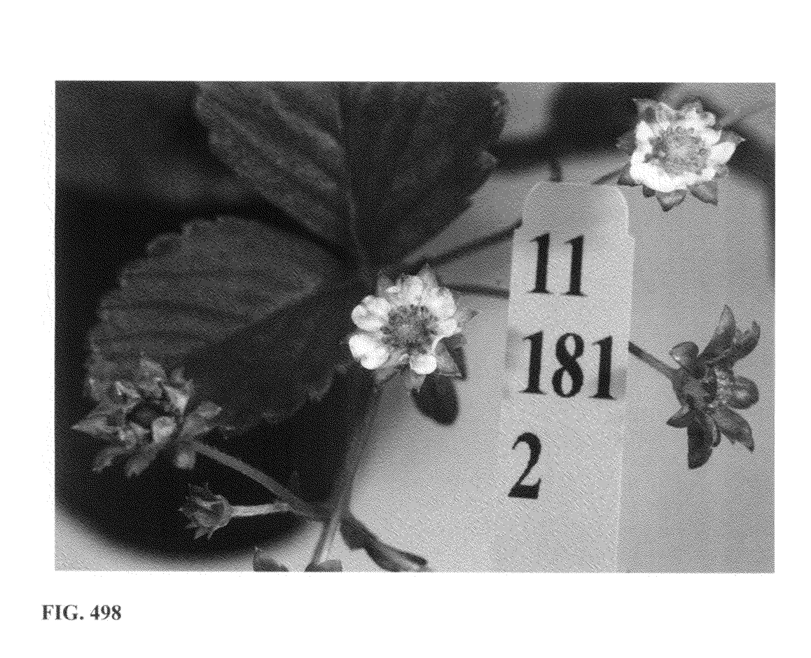

Cultivar ‘11.181-2’ has distinct disease/pathogen resistance, yield, appearance, size, and firmness. The plant of cultivar ‘11.181-2’ is characterized and distinguished in FIG. 496, the fruit of cultivar ‘11.181-2’ is characterized and distinguished in FIG. 497, and the flower of cultivar ‘11.181-2’ is characterized and distinguished in FIG. 498.

The following detailed description of cultivar ‘11.181-2’ is based on observations taken of plants and fruits (see FIGS. 496 and 497).

Certain characteristics of cultivar ‘11.181-2’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 166 provides data that characterizes the yield, appearance, size, and firmness of cultivar ‘11.181-2’.

TABLE 166

| Cultivar | Yield | App | Size | Firm |
|---|---|---|---|---|
| 11.181-2 | 2359 | 3.5 | 30.7 | 12.1 |

### Cultivar ‘11.190-3’

In certain aspects, the present invention relates to a new and distinctive day-neutral type strawberry cultivar designated as ‘11.190-3’. Cultivar ‘11.190-3’ is typical of day-neutral strawberry cultivars and produces fruit regardless of day length when treated appropriately in arid, subtropical climates.

The strawberry cultivar ‘11.190-3’ originated from a cross between two unreleased germplasm accessions ‘08.182-3’ and ‘07.216-1’. The cultivar has been asexually reproduced by runners. The properties of this cultivar were found to be transmissible by such asexual reproduction. The cultivar is stable and reproduces true to type in successive generations of asexual reproduction.

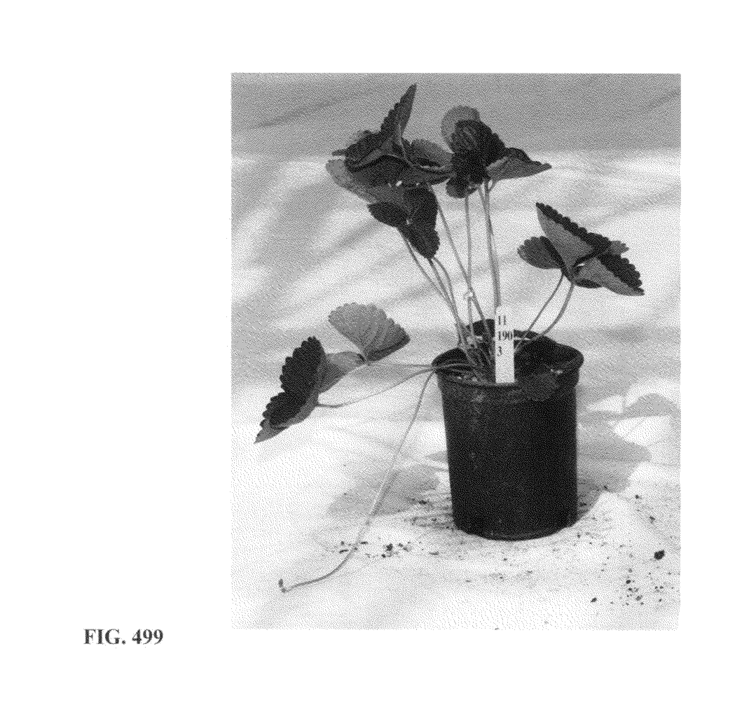

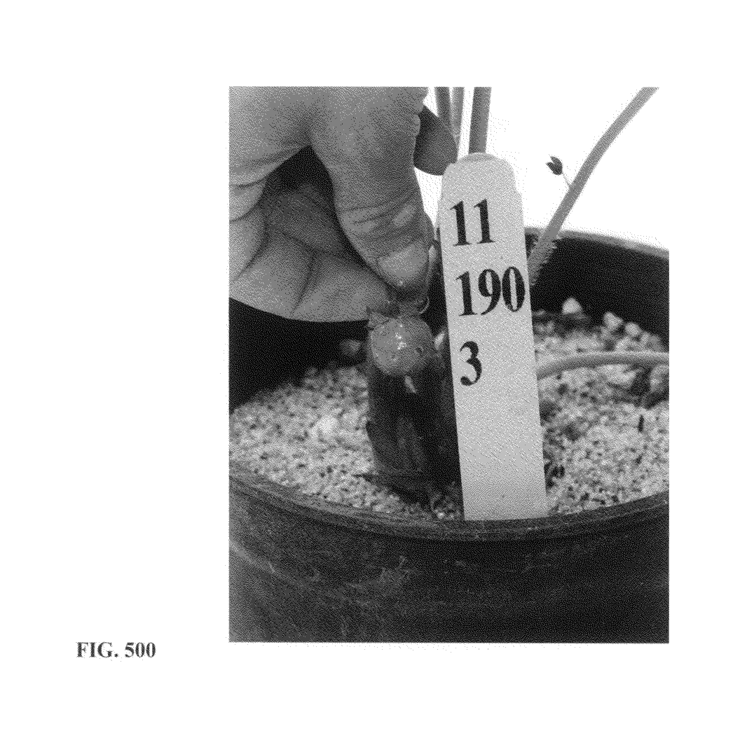

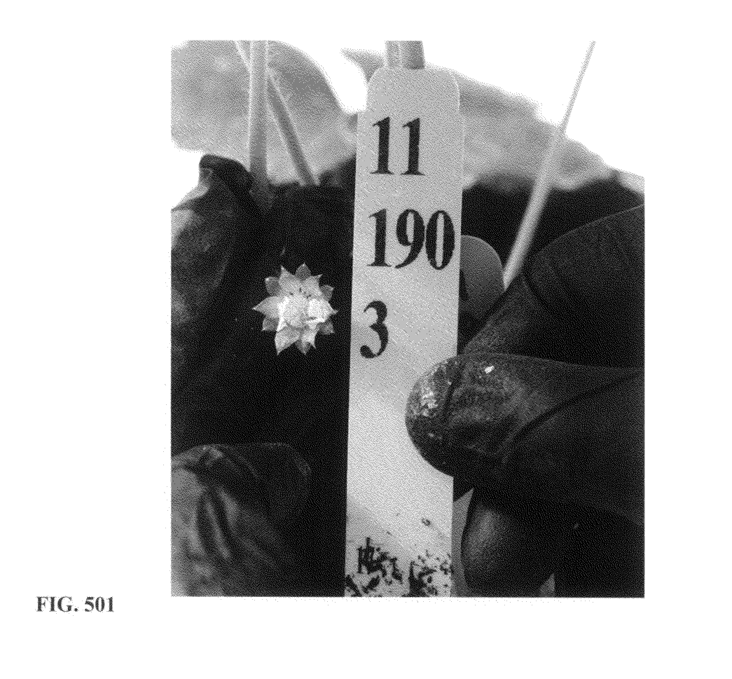

Cultivar ‘11.190-3’ has distinct disease/pathogen resistance, yield, appearance, size, and firmness. The plant of cultivar ‘11.190-3’ is characterized and distinguished in FIG. 499, the fruit of cultivar ‘11.190-3’ is characterized and distinguished in FIG. 500, and the flower of cultivar ‘11.190-3’ is characterized and distinguished in FIG. 501.

The following detailed description of cultivar ‘11.190-3’ is based on observations taken of plants and fruits (see FIGS. 499 and 500).

Certain characteristics of cultivar ‘11.190-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 167 provides data that characterizes the yield, appearance, size, and firmness of cultivar ‘11.190-3’.

TABLE 167

| Cultivar | Yield | App | Size | Firm |
|---|---|---|---|---|
| 11.190-3 | 2510 | 3.6 | 30.0 | 13.2 |

### Cultivar ‘11.195-3’

In certain aspects, the present invention relates to a new and distinctive day-neutral type strawberry cultivar designated as ‘11.195-3’. Cultivar ‘11.195-3’ is typical of day-neutral strawberry cultivars and produces fruit regardless of day length when treated appropriately in arid, subtropical climates.

The strawberry cultivar ‘11.195-3’ originated from a cross between two unreleased germplasm accessions ‘07.119-1’ and ‘07.135-1’. The cultivar has been asexually reproduced by runners. The properties of this cultivar were found to be transmissible by such asexual reproduction. The cultivar is stable and reproduces true to type in successive generations of asexual reproduction.

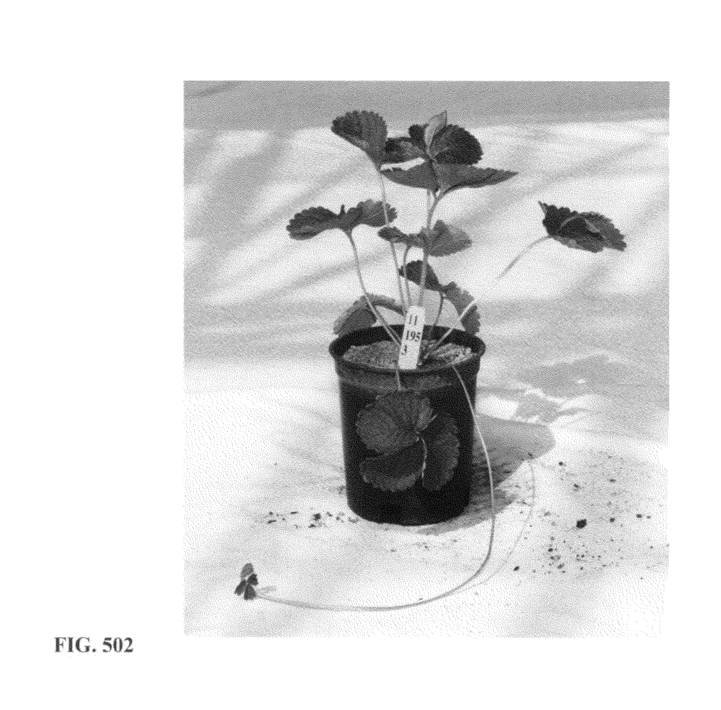

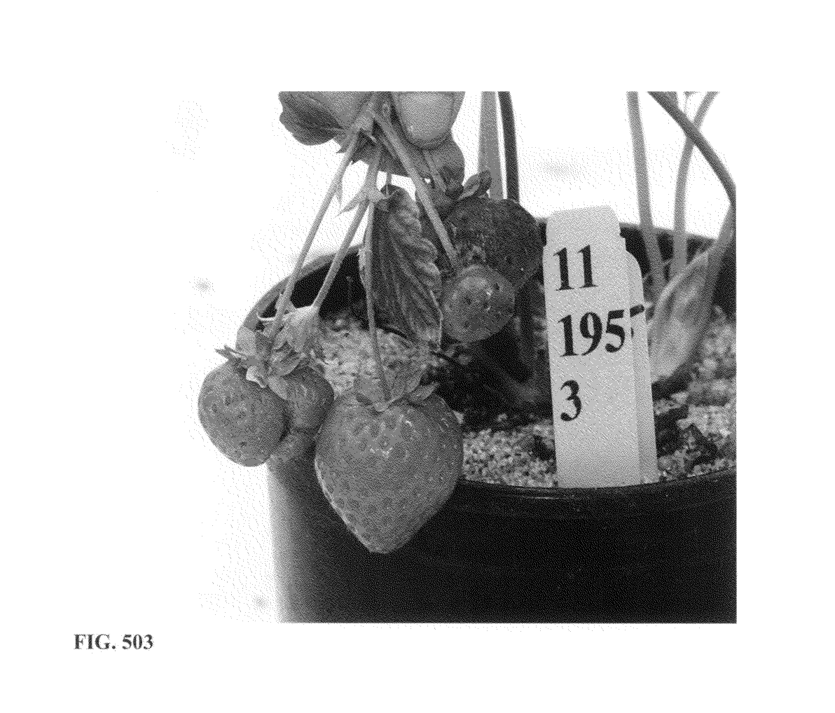

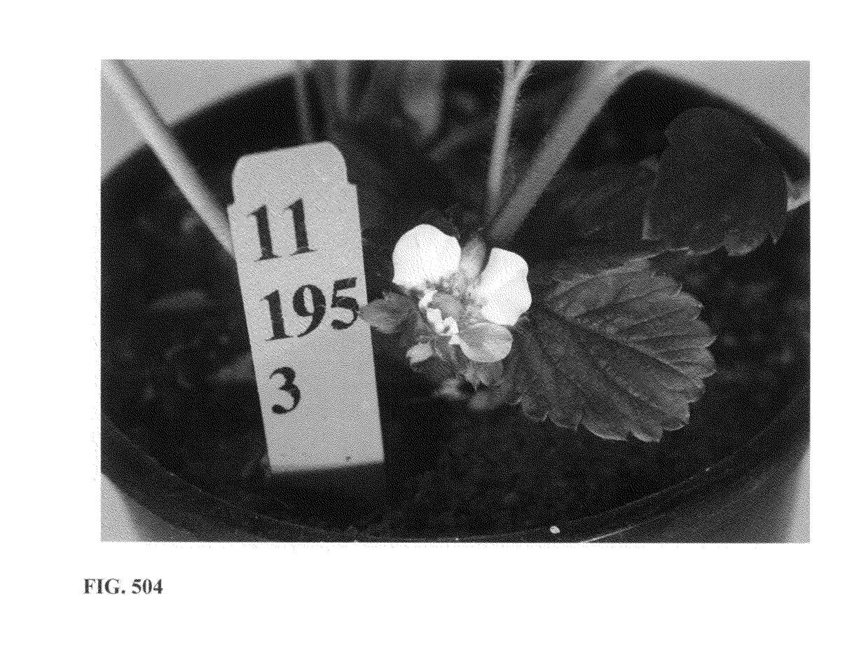

Cultivar ‘11.195-3’ has distinct disease/pathogen resistance, yield, appearance, size, and firmness. The plant of cultivar ‘11.195-3’ is characterized and distinguished in FIG. 502, the fruit of cultivar ‘11.195-3’ is characterized and distinguished in FIG. 503, and the flower of cultivar ‘11.195-3’ is characterized and distinguished in FIG. 504.

The following detailed description of cultivar ‘11.195-3’ is based on observations taken of plants and fruits (see FIGS. 502 and 503).

Certain characteristics of cultivar ‘11.195-3’ may change with changing environmental conditions (e.g., light, temperature, moisture, etc.), nutrient availability, or other factors. Quantified measurements are expressed as an average of measurements taken from a number of individual plants of the new cultivar. The measurements of any individual plant, or any group of plants, of the new cultivar may vary from the stated average.

Table 168 provides data that characterizes the yield, appearance, size, and firmness of cultivar ‘11.195-3’.

TABLE 168

| Cultivar | Yield | App | Size | Firm |
|---|---|---|---|---|
| 11.195-3 | 2578 | 3.6 | 35.6 | 10.4 |

What we claim is:

1. A new and distinct cultivar of strawberry plant as shown and described herein, wherein the cultivar is selected from the group consisting of ‘01.80-602’, ‘01.138-1’, ‘03.132-3’, ‘04.9-5’, ‘04.18-4’, ‘04.41-6’, ‘04.41-7’, ‘04.76-4’, ‘04.77-2’, ‘05.92-11’, ‘05.95-4’, ‘05.99-6’, ‘05.107-2’, ‘05.108-7’, ‘05.109-2’, ‘05.117-5’, ‘05.120-9’, ‘05.132-1’, ‘05.148-2’, ‘05.165-1’, ‘05.197-2’, ‘05.205-2’, ‘05.206-5’, ‘06.16-602’, ‘06.20-605’, ‘06.51-609’, ‘06.137-2’, ‘06.204-1’, ‘07.1-608’, ‘07.9-601’, ‘07.64-2’, ‘07.64-3’, ‘07.64-5’, ‘07.67-608’, ‘07.90-606’, ‘07.92-3’, ‘07.120-3’, ‘07.134-1’, ‘07.148-1’, ‘07.148-3’, ‘08.5-601’, ‘08.13-605’, ‘08.17-5’, ‘08.18-5’, ‘08.37-603’, ‘08.43-1’, ‘08.43-4’, ‘08.75-1’, ‘08.114-5’, ‘08.123-1’, ‘08.124-1’, ‘08.128-2’, ‘08.129-4’, ‘08.131-3’, ‘08.132-3’, ‘08.137-1’, ‘08.138-3’, ‘08.150-9’, ‘09.12-605’, ‘09.16-601’, ‘09.20-609’, ‘09.32-605’, ‘09.37-1’, ‘09.48-3’, ‘09.56-608’, ‘09.61-602’, ‘09.62-1’, ‘09.68-602’, ‘09.78-603’, ‘09.85-2’, ‘09.85-601’, ‘09.88-602’, ‘09.91-1’, ‘09.97-3’, ‘09.100-3’, ‘09.103-2’, ‘09.108-2’, ‘09.132-3’, ‘09.141-1’, ‘09.154-605’, ‘09.166-7’, ‘09.171-602’, ‘09.180-1’, ‘10.1-604’, ‘10.5-602’, ‘10.7-3’, ‘10.12-3’, ‘10.15-605’, ‘10.17-1’, ‘10.17-2’, ‘10.23-1’, ‘10.24-2’, ‘10.25-601’, ‘10.30-1’, ‘10.31-602’, ‘10.37-604’, ‘10.38-601’, ‘10.70-611’, ‘10.77-601’, ‘10.78-603’, ‘10.80-605’, ‘10.81-1’, ‘10.82-601’, ‘10.87-2’, ‘10.99-1’, ‘10.105-1’, ‘10.106-1’, ‘10.106-8’, ‘10.116-1’, ‘10.122-3’, ‘10.122-6’, ‘10.124-2’, ‘10.126-3’, ‘10.127-1’, ‘10.134-4’, ‘10.136-4’, ‘10.137-5’, ‘10.140-1’, ‘10.141-1’, ‘10.143-1’, ‘10.144-1’, ‘10.144-2’, ‘10.144-3’, ‘10.160-1’, ‘10.160-9’, ‘10.169-1’, ‘10.187-4’, ‘11.21-1’, ‘11.32-2’, ‘11.34-605’, ‘11.36-601’, ‘11.39-1’, ‘11.49-6’, ‘11.50-1’, ‘11.51-1’, ‘11.55-4’, ‘11.57-1’, ‘11.62-603’, ‘11.67-1’, ‘11.71-601’, ‘11.80-2’, ‘11.103-1’, ‘11.103-3’, ‘11.103-5’, ‘11.105-1’, ‘11.107-5’, ‘11.109-2’, ‘11.109-3’, ‘11.110-3’, ‘11.113-6111.115-3’, ‘11.116-1’, ‘11.129-2’, ‘11.141-1’, ‘11.145-1’, ‘11.151-3’, ‘11.151-8’, ‘11.153-2’, ‘11.153-3’, ‘11.154-5’, ‘11.157-1’, ‘11.158-1’, ‘11.167-4’, ‘11.179-3’, ‘11.180-1’, ‘11.181-2’, ‘11.190-3’, and ‘11.195-3’.
